(19) United States
(12) Patent Application Publication
(10) Pub. No.: US 2024/0284790 A1
(43) Pub. Date: Aug. 22, 2024

(54) HETEROCYCLIC COMPOUND, ORGANIC LIGHT-EMITTING DEVICE COMPRISING SAME, AND COMPOSITION FOR ORGANIC MATERIAL LAYER OF ORGANIC LIGHT-EMITTING DEVICE

(71) Applicant: LT MATERIALS CO.,LTD., Yongin-si, Gyeonggi-do (KR)

(72) Inventors: Hyun-Joo LEE, Yongin-si (KR); Young-Seok NO, Yongin-si (KR); Dong-Jun KIM, Yongin-si (KR)

(73) Assignee: LT MATERIALS CO.,LTD., Yongin-si, Gyeonggi-do (KR)

(21) Appl. No.: 18/563,653

(22) PCT Filed: Nov. 24, 2022
(86) PCT No.: PCT/KR2022/018727

§ 371 Date: Nov. 22, 2023

(30) Foreign Application Priority Data
Nov. 30, 2021 (KR) 10-2021-0169353

(51) Int. Cl. H10K 85/60; C07D 209/86; C07D 405/14; C07D 409/14; C09K 11/06; H10K 50/11

(52) U.S. Cl. CPC H10K 85/654; C07D 209/86; C07D 405/14; C07D 409/14; C09K 11/06; H10K 85/615; H10K 85/622; H10K 85/6572; H10K 85/6574; H10K 85/6576; C09K 2211/1018; H10K 50/11

(57) ABSTRACT

The present specification provides a heterocyclic compound, an organic light emitting device including the same and a composition for an organic material layer of the organic light emitting device. The heterocyclic compound is represented by Chemical Formula 1, and can be used as an n-type host material for the light emitting layer of an organic light emitting device by introducing various substituents around the heterocyclic compound represented by Chemical Formula 1 and changing the binding position of the substituent to adjust the bandgap.

### TECHNICAL FIELD

The present invention relates to a heterocyclic compound, an organic light emitting device including the same and a composition for an organic material layer of the organic light emitting device.

### CROSS-REFERENCE TO RELATED APPLICATIONS

This application claims priority to and the benefit of Korean Patent Application No. 10-2021-0169353 filed in the Korean Intellectual Property Office on Nov. 30, 2021, the entire contents of which are incorporated herein by reference.

### BACKGROUND ART

An organic light emitting device is a kind of self-emitting type display device, and has an advantage in that the viewing angle is wide, the contrast is excellent, and the response speed is fast.

An organic light emitting device is composed of a structure in which an organic thin film is disposed between two electrodes. When a voltage is applied to an organic light emitting device having such a structure, electrons and holes injected from the two electrodes are combined with each other in the organic thin film to make a pair, and then, the paired electrons and holes emit light while being annihilated. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, efficiency and service life of the organic light emitting device, there is a continuous need for developing a material for an organic thin film.

### RELATED ART DOCUMENTS

### Patent Document

U.S. Pat. No. 4,356,429

### DISCLOSURE

### Technical Problem

The present invention has been made in an effort to provide a heterocyclic compound, an organic light emitting device including the same and a composition for an organic material layer of the organic light emitting device.

### Technical Solution

In an exemplary embodiment of the present application, provided is a heterocyclic compound represented by the following Chemical Formula 1.

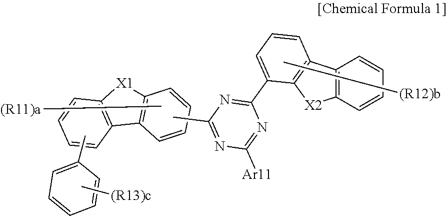

In Chemical Formula 1,
X1 and X2 are the same as or different from each other, and are each independently O; or S,
Ar11 is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R11 to R13 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
a is an integer from 0 to 6, and when a is 2 or higher, R11's are the same as or different from each other,
b is an integer from 0 to 7, and when b is 2 or higher, R12's are the same as or different from each other, and
c is an integer from 0 to 5, and when c is 2 or higher, R13's are the same as or different from each other.

Further, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In addition, in an exemplary embodiment of the present application, provided is an organic light emitting device in which the organic material layer further includes a heterocyclic compound represented by the following Chemical Formula 2.

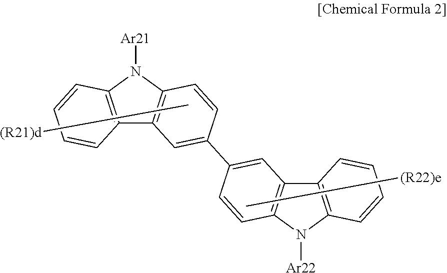

In Chemical Formula 2,
Ar21 and Ar22 are the same as or different from each other, and are each independently a substituted or unsubstituted C6 to C60 aryl group; or a substituted or unsubstituted C2 to C60 heteroaryl group,
R21 and R22 are the same as or different from each other, and are each independently selected from the group consisting of hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more adjacent groups are bonded to each other to form a substituted or unsubstituted aliphatic or aromatic hydrocarbon ring, and
d and e are an integer from 0 to 7, and when d and e are 2 or higher, substituents in the parenthesis are the same as or different from each other.

Finally, in an exemplary embodiment of the present application, provided is a composition for an organic material layer of an organic light emitting device, which includes the heterocyclic compound represented by Chemical Formula 1; and the heterocyclic compound represented by Chemical Formula 2.

### Advantageous Effects

The heterocyclic compound described in the present specification may be used as a material for the organic material layer of the organic light emitting device. That is, the heterocyclic compound can serve as a light emitting material, a hole injection material, a hole transport material, an electron transport material, an electron injection material and the like in the organic light emitting device. In particular, the heterocyclic compound can be used as a material for a light emitting layer of an organic light emitting device.

Specifically, one or two or more of the heterocyclic compounds represented by Chemical Formula 1 can be used, and the heterocyclic compounds represented by Chemical Formula 1 can be used as materials for the light emitting layer. In particular, the heterocyclic compound can be used as an n-type host material for the light emitting layer of an organic light emitting device by introducing various substituents and changing the binding position of the substituent to adjust the bandgap.

Furthermore, the heterocyclic compound represented by Chemical Formula 1 can be applied to the organic material layer of the organic light emitting device as a combination of two types by further including the heterocyclic compound represented by Chemical Formula 2 as a p-type host material of the light emitting layer.

### DESCRIPTION OF DRAWINGS

FIGS. 1 to 3 are views each schematically illustrating a stacking structure of an organic light emitting device according to an exemplary embodiment of the present application.

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
400: Negative electrode

### MODE FOR INVENTION

Hereinafter, the present specification will be described in more detail.

When one part “includes” one constituent element in the present specification, unless otherwise specifically described, this does not mean that another constituent element is excluded, but means that another constituent element may be further included.

In the present specification,

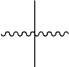

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and the isotope may be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

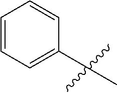 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

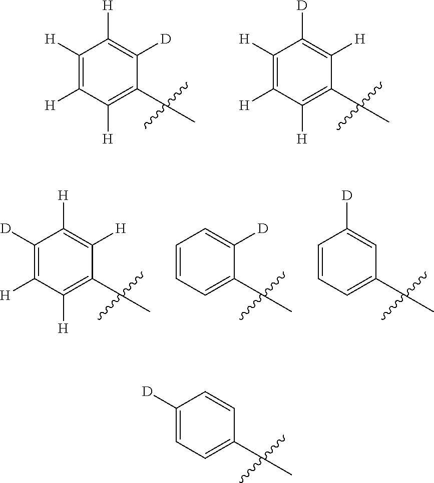 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

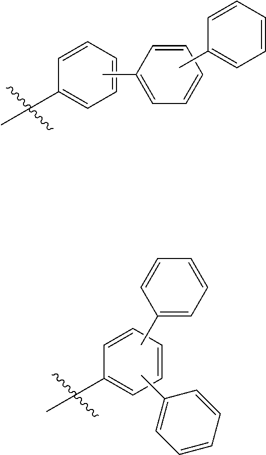

In the present specification, the fluorenyl group may be substituted, and adjacent substituents may be bonded to each other to form a ring.

When the fluorenyl group is substituted, the substituent may be any one of the following structures, but is not limited thereto.

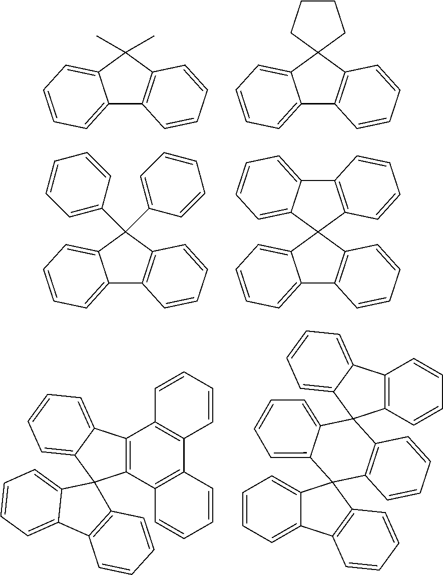

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole) group, a dihydrophenazine group, a phenoxazine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiathiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, when the substituent is a carbazole group, it means being bonded to nitrogen or carbon of carbazole.

In the present specification, when a carbazole group is substituted, an additional substituent may be substituted with the nitrogen or carbon of the carbazole.

In the present specification, a benzocarbazole group may be any one of the following structures.

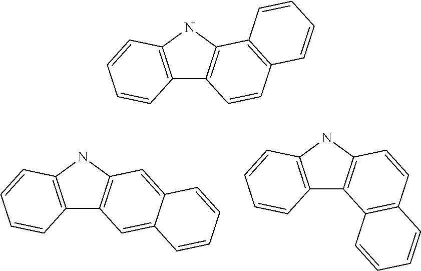

In the present specification, a dibenzocarbazole group may be any one of the following structures.

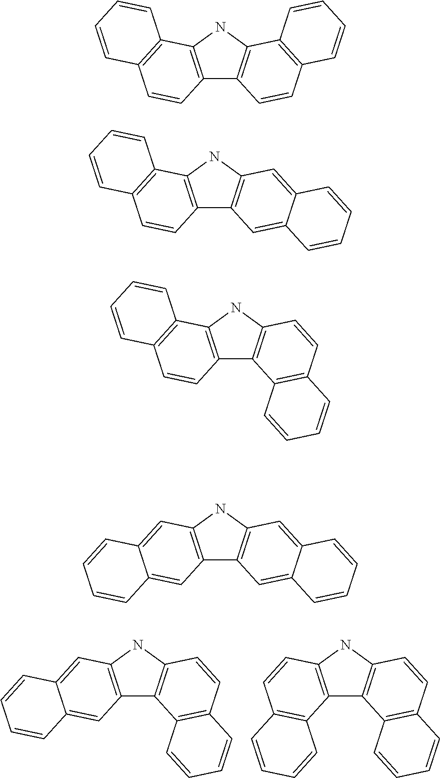

In the present specification, a naphthobenzofuran group may be any one of the following structures.

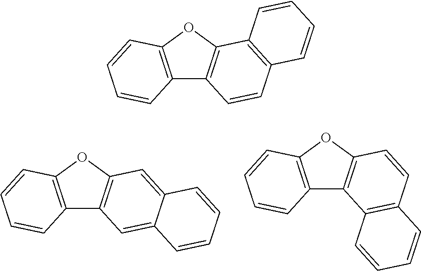

In the present specification, a naphthobenzothiophene group may be any one of the following structures.

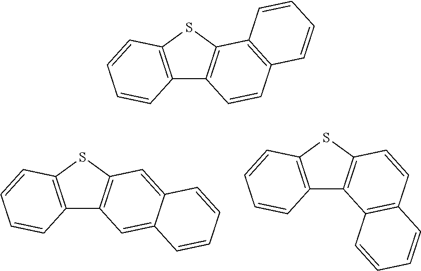

In the present specification, a silyl group includes Si and is a substituent to which the Si atom is directly linked as a radical, and is represented by —Si(R107) (R108) (R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

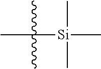 [SMILES: *[Si](C)(C)C]

(a trimethylsilyl group),

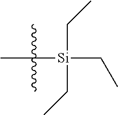 [SMILES: *[Si](CC)(CC)CC]

(a triethylsilyl group),

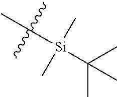 [SMILES: *[Si](C)(C)C(C)(C)C]

(a t-butyldimethylsilyl group),

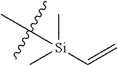 [SMILES: *[Si](C)(C)C=C]

(a vinyldimethylsilyl group),

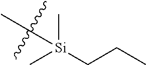 [SMILES: *[Si](C)(C)CCC]

(a propyldimethylsilyl group),

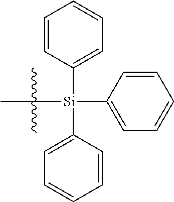 [SMILES: *[Si](c1ccccc1)(c1ccccc1)c1ccccc1]

(a triphenylsilyl group),

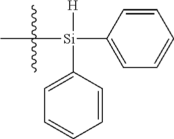 [SMILES: *[SiH](c1ccccc1)c1ccccc1]

(a diphenylsilyl group),

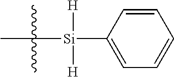 [SMILES: *[SiH2]c1ccccc1]

(a phenylsilyl group) and the like, but are not limited thereto.

In the present specification, a phosphine oxide group is represented by —P(═O) (R110) (R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide group, and the like, but are not limited thereto.

In the present specification, an amine group is represented by —N(R112) (R113), and R112 and R113 are the same as or different from each other, and are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be applied to the rings, except for those that are not monovalent groups.

In an exemplary embodiment of the present application, provided is the compound represented by Chemical Formula 1.

In an exemplary embodiment of the present application, a group not represented by a substituent; or a group represented by hydrogen may mean being all substitutable with deuterium. That is, it may be shown that hydrogen; or deuterium can be substituted with each other.

In an exemplary embodiment of the present application, the deuterium content of the heterocyclic compound of Chemical Formula 1 may be 0% to 100%.

In general, compounds bonded with hydrogen and compounds substituted with deuterium exhibit a difference in thermodynamic behavior. The reason for this is that the mass of a deuterium atom is 2-fold higher than that of hydrogen, but due to the difference in the mass of atoms, deuterium is characterized by having even lower vibration energy. In addition, the bond length of carbon and deuterium is shorter than that of a bond with hydrogen, and a dissociation energy used to break the bond is also stronger than that of the bond with hydrogen. This is because the van der Waals radius of deuterium is smaller than that of hydrogen, and thus the extension amplitude of a bond between carbon and deuterium becomes even narrower.

The deuterium-substituted compound in the heterocyclic compound of Chemical Formula 1 of the present invention is characterized in that the energy in the ground state is further lower than that of the hydrogen-substituted compound, and the shorter the bond length between carbon and deuterium is, the smaller the molecular hardcore volume is. Accordingly, the electrical polarizability may be reduced and the intermolecular interaction can be weakened, so that the volume of the device thin film may be increased. These characteristics induce an effect of lowering the crystallinity by creating the amorphous state of a thin film. Therefore, deuterium substitution in the heterocyclic compound of Chemical Formula 1 may be effective in improving the heat resistance of an OLED device, thereby improving the service life and driving characteristics.

In an exemplary embodiment of the present application, Chemical Formula 1 may be represented by any one of the following Chemical Formulae 1-1 to 1-3.

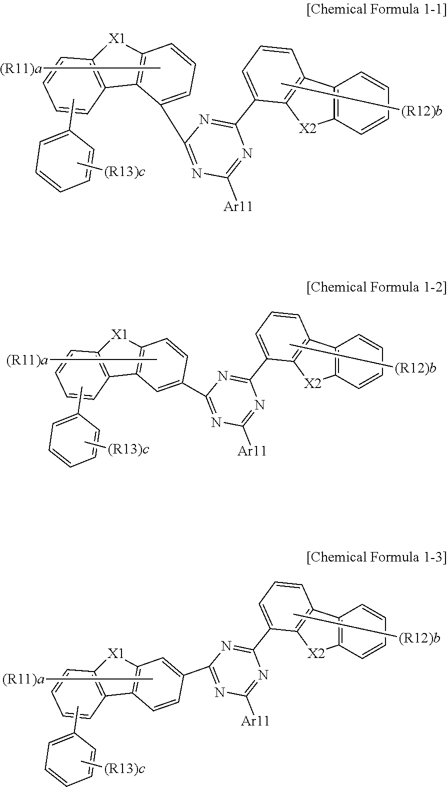

In Chemical Formulae 1-1 to 1-3,
the definitions of X1, X2, Ar11, R11 to R13, a, b and c are the same as the definitions in Chemical Formula 1.

In an exemplary embodiment of the present application, Chemical Formula 1 may be represented by any one of Chemical Formula 1-A to 1-D.

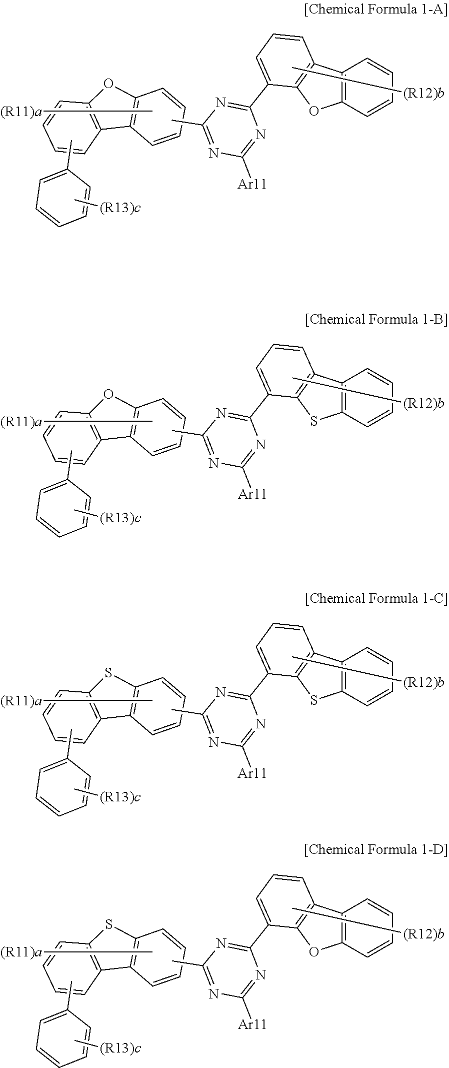

In Chemical Formulae 1-A to 1-D,
the definitions of Ar11, R11 to R13, a, b and c are the same as the definitions in Chemical Formula 1.

In an exemplary embodiment of the present application, X1 and X2 are the same as or different from each other, and may be each independently O; or S.

In another exemplary embodiment, both X1 and X2 may be O.

In still another exemplary embodiment, X1 may be O, and X2 may be S.

In yet another exemplary embodiment, both X1 and X2 may be S.

In yet another exemplary embodiment, X1 may be S, and X2 may be O.

In an exemplary embodiment of the present application, Ar11 may be a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another exemplary embodiment, Ar11 may be a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another exemplary embodiment, Ar11 may be a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In yet another exemplary embodiment, Ar11 may be a substituted or unsubstituted C6 to C20 aryl group.

In yet another exemplary embodiment, Ar11 may be a substituted or unsubstituted phenyl group; a substituted or unsubstituted biphenyl group; a substituted or unsubstituted terphenyl group; or a substituted or unsubstituted fluorene group.

In yet another exemplary embodiment, Ar11 may be a phenyl group which is unsubstituted or substituted with deuterium; a biphenyl group which is unsubstituted or substituted with deuterium; a terphenyl group which is unsubstituted or substituted with deuterium; or a fluorene group which is unsubstituted or substituted with deuterium.

In an exemplary embodiment of the present application, R11 to R13 are the same as or different from each other, and may be each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another exemplary embodiment, R11 to R13 are the same as or different from each other, and may be each independently hydrogen; deuterium; a halogen group; a cyano group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another exemplary embodiment, R11 to R13 are the same as or different from each other, and may be each independently hydrogen; deuterium; a halogen group; a cyano group; a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In yet another exemplary embodiment, R11 to R13 are the same as or different from each other, and may be each independently hydrogen; deuterium; a halogen group; a cyano group; a substituted or unsubstituted C1 to C10 alkyl group; a substituted or unsubstituted C3 to C10 cycloalkyl group; a substituted or unsubstituted C2 to C10 heterocycloalkyl group; a substituted or unsubstituted C6 to C10 aryl group; or a substituted or unsubstituted C2 to C10 heteroaryl group.

In yet another exemplary embodiment, R11 to R13 are the same as or different from each other, and may be each independently hydrogen; or deuterium.

In an exemplary embodiment of the present application, Chemical Formula 1 is dividedly represented by the following Structures 1a to 1c, and the deuterium content of the following Structures 1a to 1c may be each 0% to 100%.

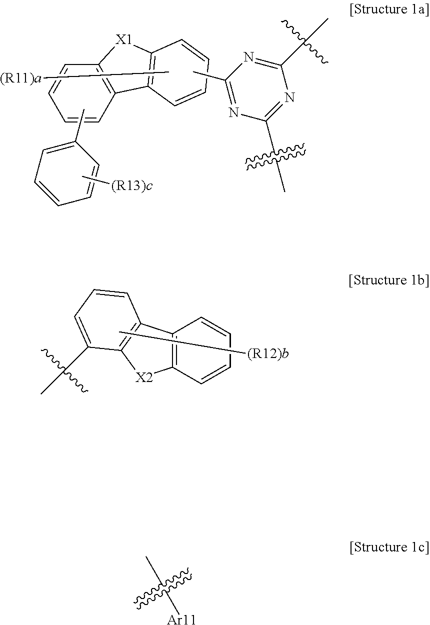

In Structures 1a to 1c,
the definitions of X1, X2, Ar11, R11 to R13, a, b and c are the same as the definitions in Chemical Formula 1, and

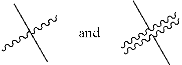

are each a binding position where those which are the same are linked to each other.

In another exemplary embodiment, the deuterium content of Structure 1a may be 0% to 100%.

In still another exemplary embodiment, the deuterium content of Structure 1a may be 0%, or 20% to 100%.

In yet another exemplary embodiment, the deuterium content of Structure 1a may be 0%, or 40% to 100%.

In yet another exemplary embodiment, the deuterium content of Structure 1a may be 0%, or 60% to 100%.

In yet another exemplary embodiment, the deuterium content of Structure 1a may be 0%, or 80% to 100%.

In yet another exemplary embodiment, the deuterium content of Structure 1a may be 0% or 100%.

In yet another exemplary embodiment, the deuterium content of Structure 1b may be 0% to 100%.

In yet another exemplary embodiment, the deuterium content of Structure 1b may be 0%, or 20% to 100%.

In yet another exemplary embodiment, the deuterium content of Structure 1b may be 0%, or 40% to 100%.

In yet another exemplary embodiment, the deuterium content of Structure 1b may be 0%, or 60% to 100%.

In yet another exemplary embodiment, the deuterium content of Structure 1b may be 0%, or 80% to 100%.

In yet another exemplary embodiment, the deuterium content of Structure 1b may be 0% or 100%.

In yet another exemplary embodiment, the deuterium content of Structure 1c may be 0% to 100%.

In yet another exemplary embodiment, the deuterium content of Structure 1c may be 0%, or 20% to 100%.

In yet another exemplary embodiment, the deuterium content of Structure 1c may be 0%, or 40% to 100%.

In yet another exemplary embodiment, the deuterium content of Structure 1c may be 0%, or 60% to 100%.

In yet another exemplary embodiment, the deuterium content of Structure 1c may be 0%, or 80% to 100%.

In yet another exemplary embodiment, the deuterium content of Structure 1c may be 0% or 100%.

In an exemplary embodiment of the present application, provided is a heterocyclic compound in which Chemical Formula 1 is represented by any one of the following compounds.

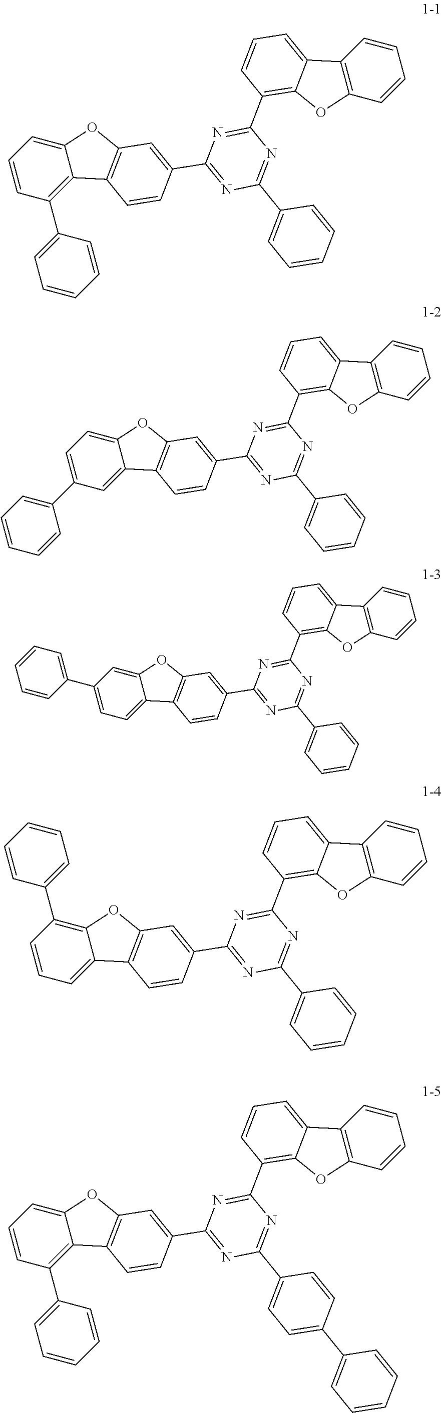

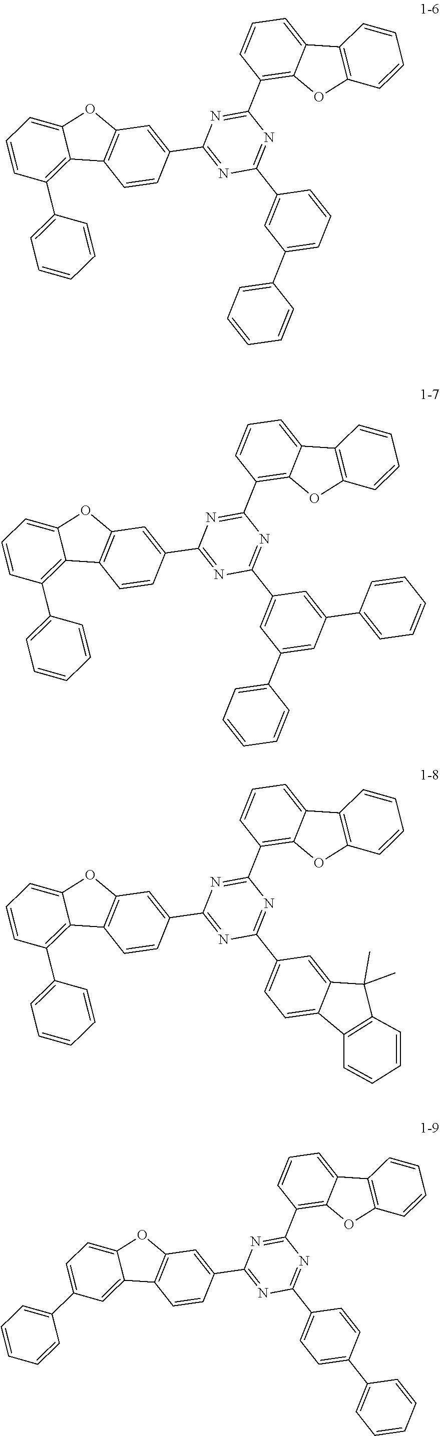

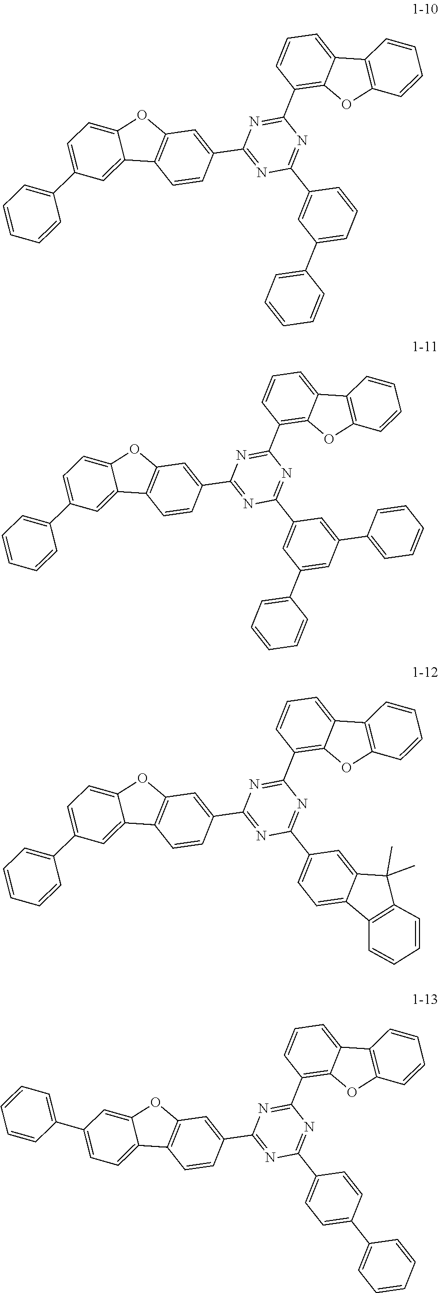 [SMILES: c1ccc(-c2cccc(-c3nc(-c4ccc5c(c4)oc4ccc(-c6ccccc6)cc45)nc(-c4cccc5c4oc4ccccc45)n3)c2)cc1 ; c1ccc(-c2cc(-c3ccccc3)cc(-c3nc(-c4ccc5c(c4)oc4ccc(-c6ccccc6)cc45)nc(-c4cccc5c4oc4ccccc45)n3)c2)cc1 ; CC1(C)c2ccccc2-c2ccc(-c3nc(-c4ccc5c(c4)oc4ccc(-c6ccccc6)cc45)nc(-c4cccc5c4oc4ccccc45)n3)cc21 ; c1ccc(-c2ccc(-c3nc(-c4ccc5c(c4)oc4cc(-c6ccccc6)ccc45)nc(-c4cccc5c4oc4ccccc45)n3)cc2)cc1]

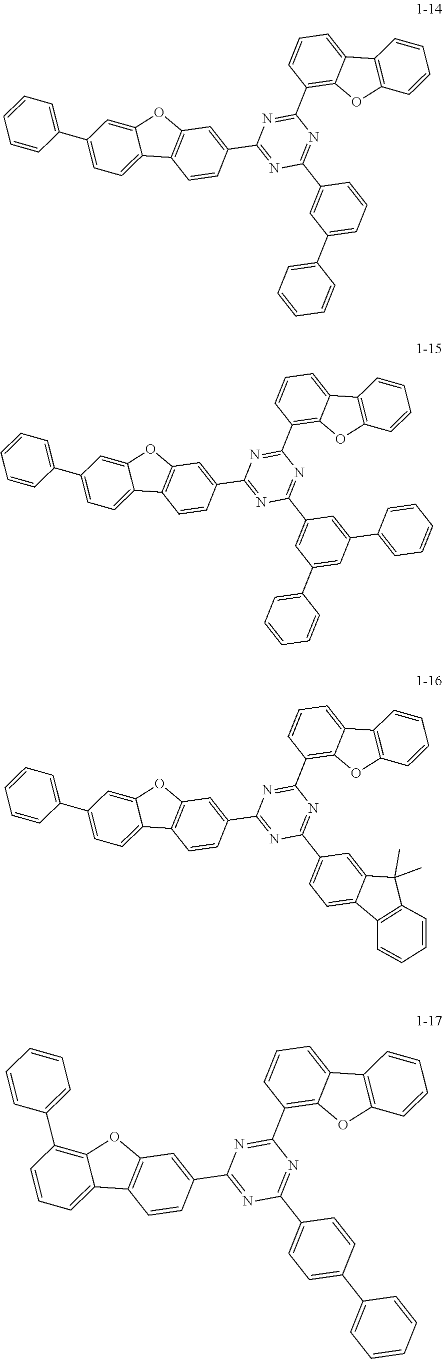 [SMILES: c1ccc(-c2cccc(-c3nc(-c4ccc5c(c4)oc4cc(-c6ccccc6)ccc45)nc(-c4cccc5c4oc4ccccc45)n3)c2)cc1 ; c1ccc(-c2cc(-c3ccccc3)cc(-c3nc(-c4ccc5c(c4)oc4cc(-c6ccccc6)ccc45)nc(-c4cccc5c4oc4ccccc45)n3)c2)cc1 ; CC1(C)c2ccccc2-c2ccc(-c3nc(-c4ccc5c(c4)oc4cc(-c6ccccc6)ccc45)nc(-c4cccc5c4oc4ccccc45)n3)cc21 ; c1ccc(-c2ccc(-c3nc(-c4ccc5c(c4)oc4c(-c6ccccc6)cccc45)nc(-c4cccc5c4oc4ccccc45)n3)cc2)cc1]

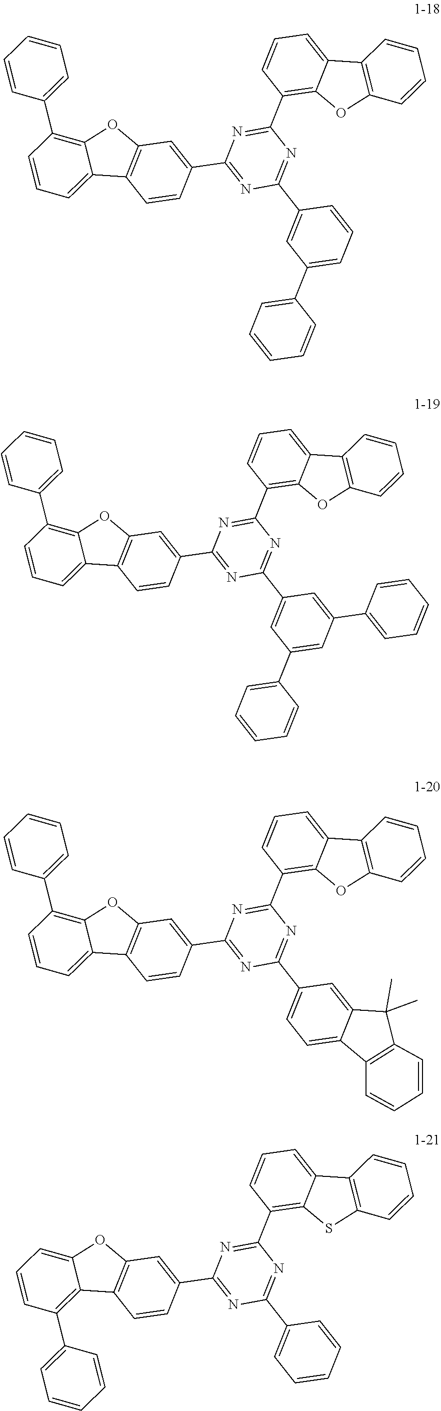 [SMILES: c1ccc(-c2cccc(-c3nc(-c4ccc5c(c4)oc4c(-c6ccccc6)cccc45)nc(-c4cccc5c4oc4ccccc45)n3)c2)cc1 ; c1ccc(-c2cc(-c3ccccc3)cc(-c3nc(-c4ccc5c(c4)oc4c(-c6ccccc6)cccc45)nc(-c4cccc5c4oc4ccccc45)n3)c2)cc1 ; CC1(C)c2ccccc2-c2ccc(-c3nc(-c4ccc5c(c4)oc4c(-c6ccccc6)cccc45)nc(-c4cccc5c4oc4ccccc45)n3)cc21 ; c1ccc(-c2nc(-c3ccc4c(c3)oc3cccc(-c5ccccc5)c34)nc(-c3cccc4c3sc3ccccc34)n2)cc1]

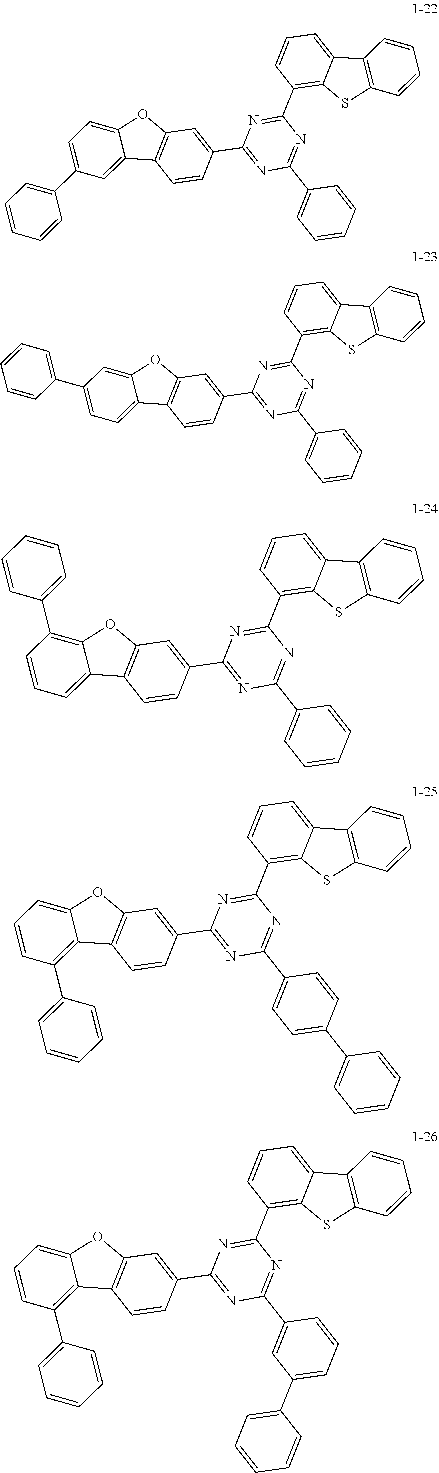

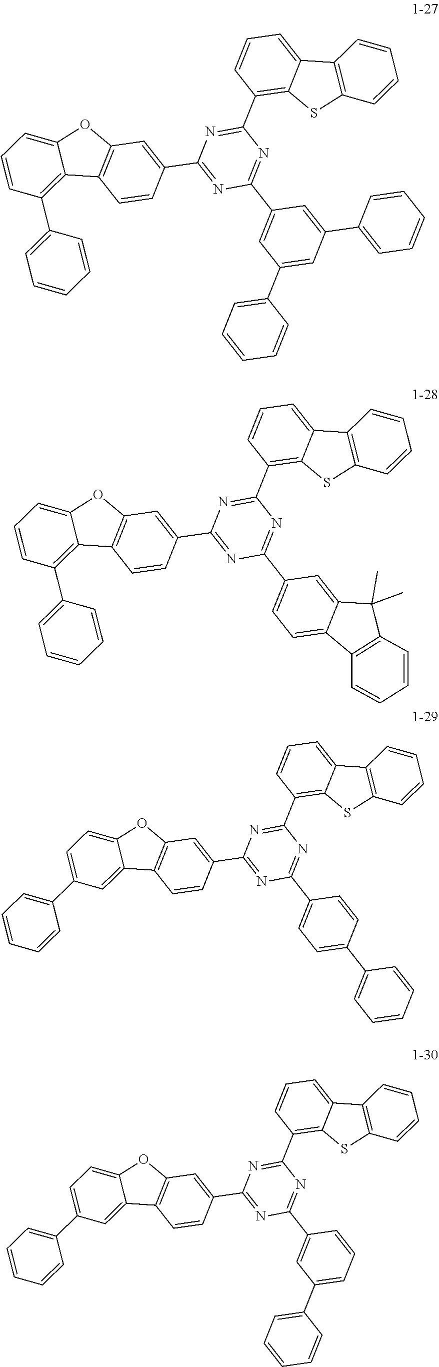

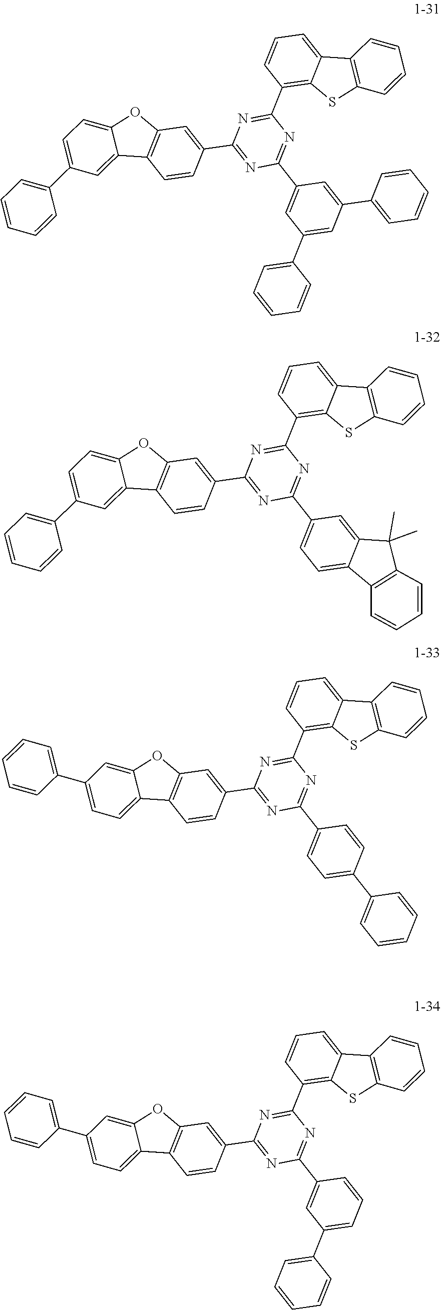 [SMILES: c1ccc(-c2cc(-c3ccccc3)cc(-c3nc(-c4ccc5c(c4)oc4ccc(-c6ccccc6)cc45)nc(-c4cccc5c4sc4ccccc45)n3)c2)cc1 ; CC1(C)c2ccccc2-c2ccc(-c3nc(-c4ccc5c(c4)oc4ccc(-c6ccccc6)cc45)nc(-c4cccc5c4sc4ccccc45)n3)cc21 ; c1ccc(-c2ccc(-c3nc(-c4ccc5c(c4)oc4cc(-c6ccccc6)ccc45)nc(-c4cccc5c4sc4ccccc45)n3)cc2)cc1 ; c1ccc(-c2cccc(-c3nc(-c4ccc5c(c4)oc4cc(-c6ccccc6)ccc45)nc(-c4cccc5c4sc4ccccc45)n3)c2)cc1]

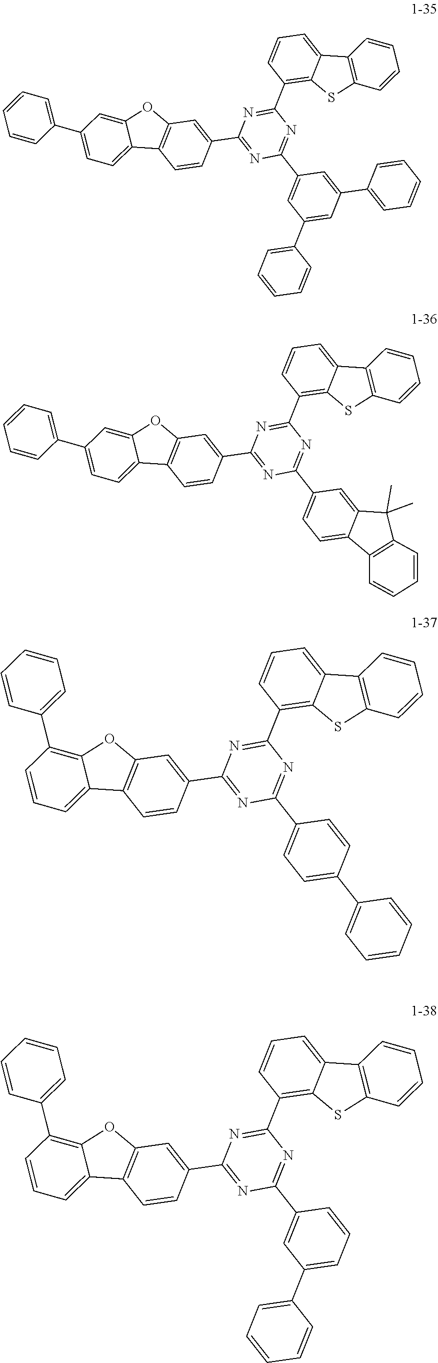 [SMILES: c1ccc(-c2cc(-c3ccccc3)cc(-c3nc(-c4ccc5c(c4)oc4cc(-c6ccccc6)ccc45)nc(-c4cccc5c4sc4ccccc45)n3)c2)cc1 ; CC1(C)c2ccccc2-c2ccc(-c3nc(-c4ccc5c(c4)oc4cc(-c6ccccc6)ccc45)nc(-c4cccc5c4sc4ccccc45)n3)cc21 ; c1ccc(-c2ccc(-c3nc(-c4ccc5c(c4)oc4c(-c6ccccc6)cccc45)nc(-c4cccc5c4sc4ccccc45)n3)cc2)cc1 ; c1ccc(-c2cccc(-c3nc(-c4ccc5c(c4)oc4c(-c6ccccc6)cccc45)nc(-c4cccc5c4sc4ccccc45)n3)c2)cc1]

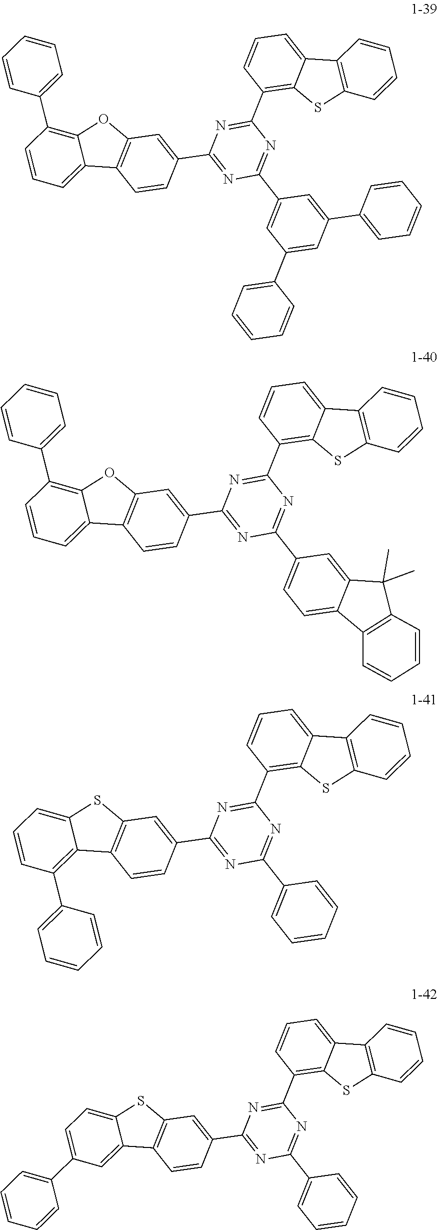

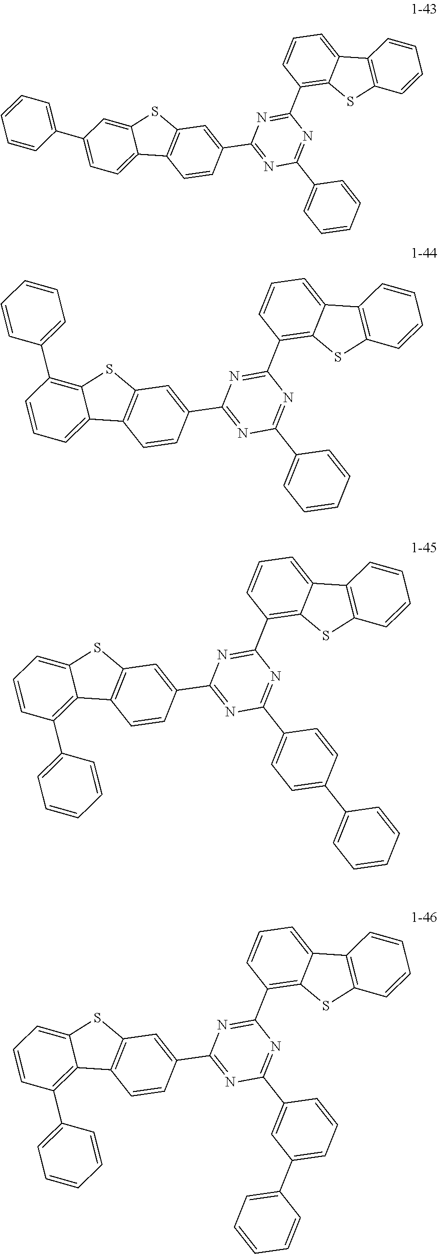 [SMILES: c1ccc(-c2ccc3c(c2)sc2cc(-c4nc(-c5ccccc5)nc(-c5cccc6c5sc5ccccc56)n4)ccc23)cc1 ; c1ccc(-c2nc(-c3ccc4c(c3)sc3c(-c5ccccc5)cccc34)nc(-c3cccc4c3sc3ccccc34)n2)cc1 ; c1ccc(-c2ccc(-c3nc(-c4ccc5c(c4)sc4cccc(-c6ccccc6)c45)nc(-c4cccc5c4sc4ccccc45)n3)cc2)cc1 ; c1ccc(-c2cccc(-c3nc(-c4ccc5c(c4)sc4cccc(-c6ccccc6)c45)nc(-c4cccc5c4sc4ccccc45)n3)c2)cc1]

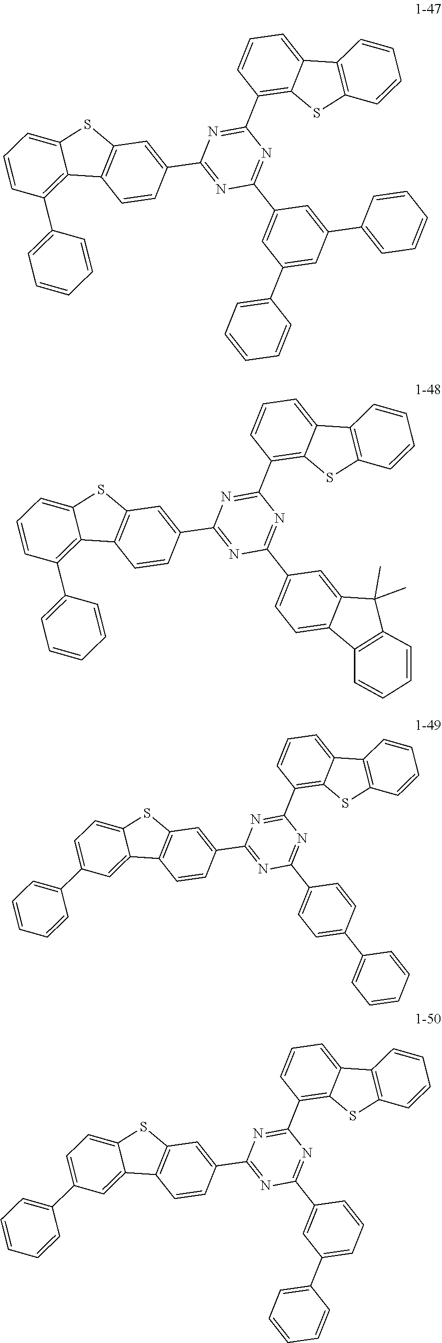

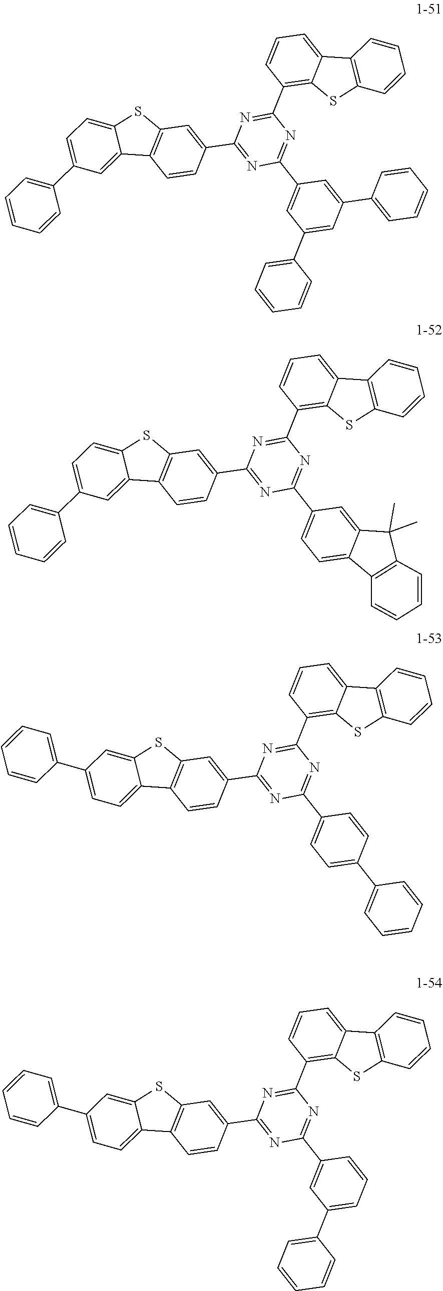 [SMILES: c1ccc(-c2cc(-c3ccccc3)cc(-c3nc(-c4ccc5c(c4)sc4ccc(-c6ccccc6)cc45)nc(-c4cccc5c4sc4ccccc45)n3)c2)cc1 ; CC1(C)c2ccccc2-c2ccc(-c3nc(-c4ccc5c(c4)sc4ccc(-c6ccccc6)cc45)nc(-c4cccc5c4sc4ccccc45)n3)cc21 ; c1ccc(-c2ccc(-c3nc(-c4ccc5c(c4)sc4cc(-c6ccccc6)ccc45)nc(-c4cccc5c4sc4ccccc45)n3)cc2)cc1 ; c1ccc(-c2cccc(-c3nc(-c4ccc5c(c4)sc4cc(-c6ccccc6)ccc45)nc(-c4cccc5c4sc4ccccc45)n3)c2)cc1]

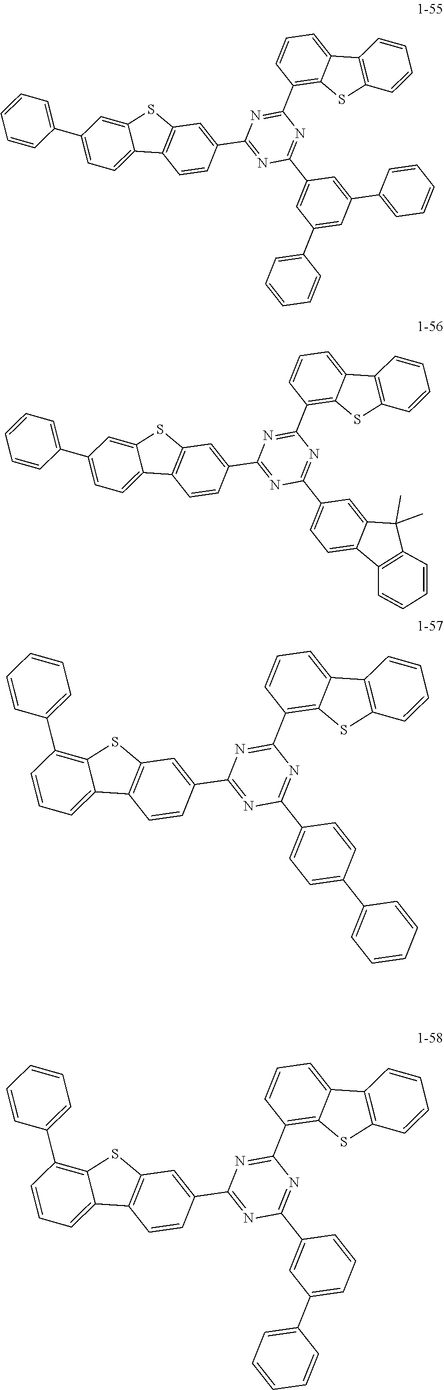 [SMILES: c1ccc(-c2cc(-c3ccccc3)cc(-c3nc(-c4ccc5c(c4)sc4cc(-c6ccccc6)ccc45)nc(-c4cccc5c4sc4ccccc45)n3)c2)cc1 ; CC1(C)c2ccccc2-c2ccc(-c3nc(-c4ccc5c(c4)sc4cc(-c6ccccc6)ccc45)nc(-c4cccc5c4sc4ccccc45)n3)cc21 ; c1ccc(-c2ccc(-c3nc(-c4ccc5c(c4)sc4c(-c6ccccc6)cccc45)nc(-c4cccc5c4sc4ccccc45)n3)cc2)cc1 ; c1ccc(-c2cccc(-c3nc(-c4ccc5c(c4)sc4c(-c6ccccc6)cccc45)nc(-c4cccc5c4sc4ccccc45)n3)c2)cc1]

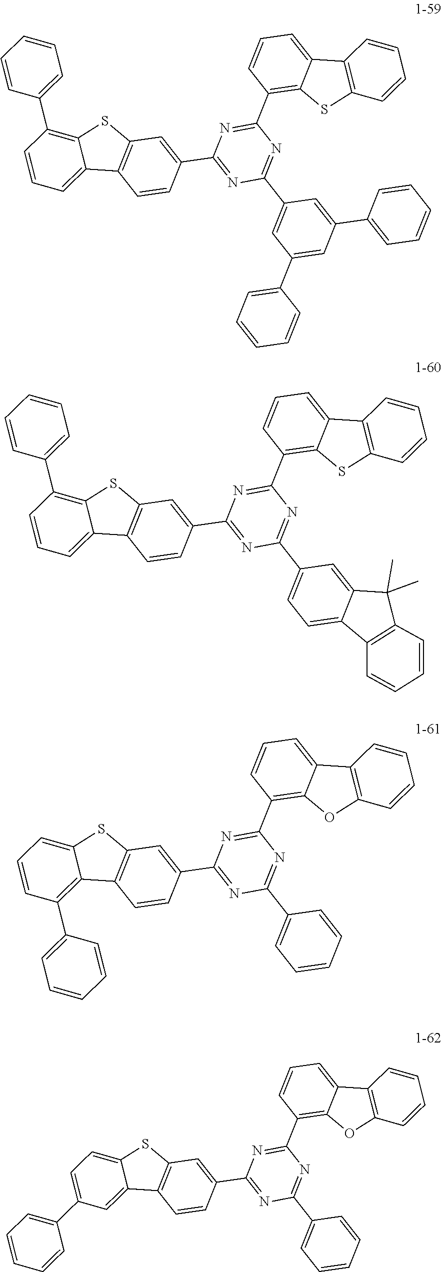 [SMILES: c1ccc(-c2cc(-c3ccccc3)cc(-c3nc(-c4ccc5c(c4)sc4c(-c6ccccc6)cccc45)nc(-c4cccc5c4sc4ccccc45)n3)c2)cc1 ; CC1(C)c2ccccc2-c2ccc(-c3nc(-c4ccc5c(c4)sc4c(-c6ccccc6)cccc45)nc(-c4cccc5c4sc4ccccc45)n3)cc21 ; c1ccc(-c2nc(-c3ccc4c(c3)sc3cccc(-c5ccccc5)c34)nc(-c3cccc4c3oc3ccccc34)n2)cc1 ; c1ccc(-c2ccc3sc4cc(-c5nc(-c6ccccc6)nc(-c6cccc7c6oc6ccccc67)n5)ccc4c3c2)cc1]

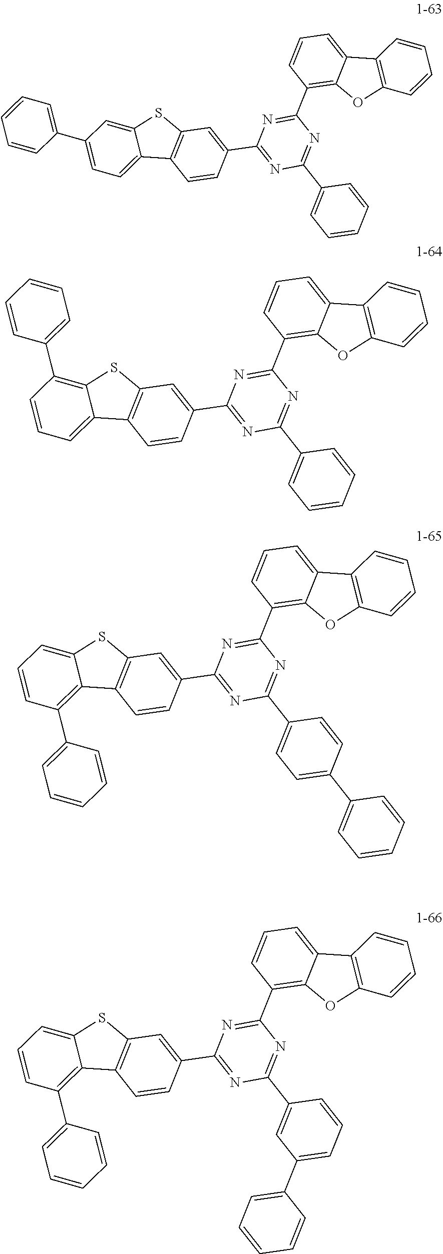

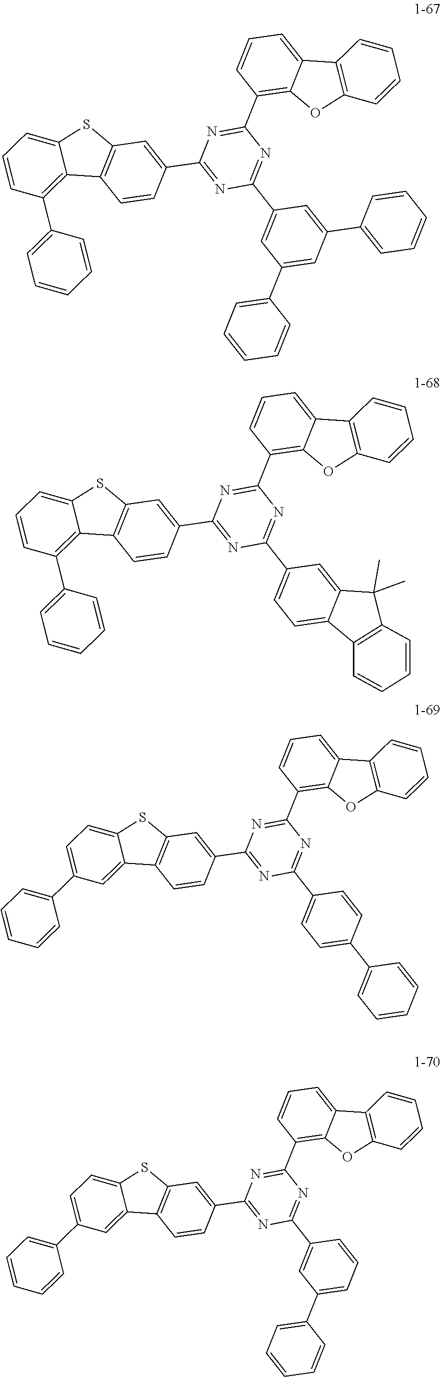

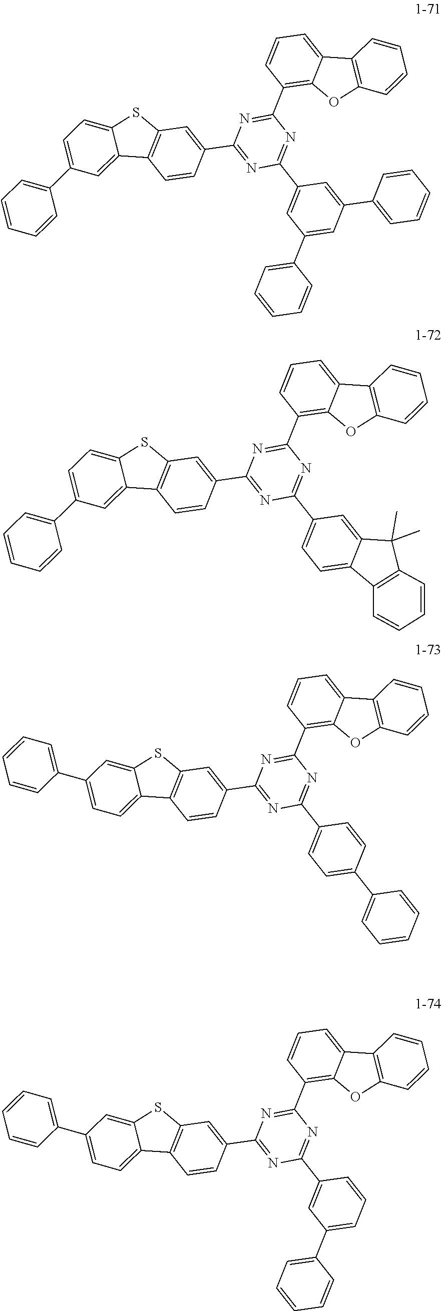 [SMILES: c1ccc(-c2cc(-c3ccccc3)cc(-c3nc(-c4ccc5c(c4)sc4ccc(-c6ccccc6)cc45)nc(-c4cccc5c4oc4ccccc45)n3)c2)cc1 ; CC1(C)c2ccccc2-c2ccc(-c3nc(-c4ccc5c(c4)sc4ccc(-c6ccccc6)cc45)nc(-c4cccc5c4oc4ccccc45)n3)cc21 ; c1ccc(-c2ccc(-c3nc(-c4ccc5c(c4)sc4cc(-c6ccccc6)ccc45)nc(-c4cccc5c4oc4ccccc45)n3)cc2)cc1 ; c1ccc(-c2cccc(-c3nc(-c4ccc5c(c4)sc4cc(-c6ccccc6)ccc45)nc(-c4cccc5c4oc4ccccc45)n3)c2)cc1]

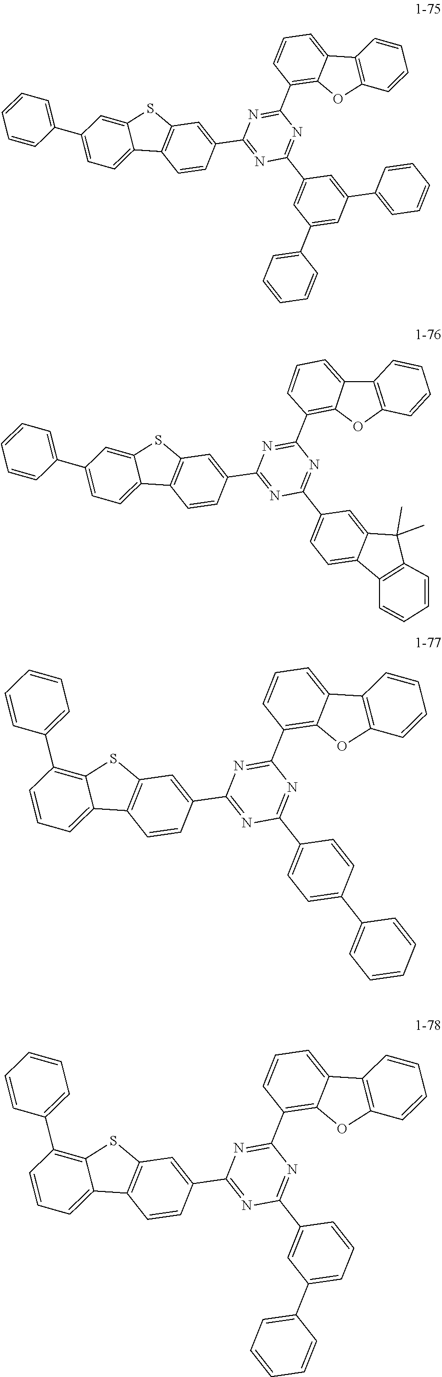 [SMILES: c1ccc(-c2cc(-c3ccccc3)cc(-c3nc(-c4ccc5c(c4)sc4cc(-c6ccccc6)ccc45)nc(-c4cccc5c4oc4ccccc45)n3)c2)cc1 ; CC1(C)c2ccccc2-c2ccc(-c3nc(-c4ccc5c(c4)sc4cc(-c6ccccc6)ccc45)nc(-c4cccc5c4oc4ccccc45)n3)cc21 ; c1ccc(-c2ccc(-c3nc(-c4ccc5c(c4)sc4c(-c6ccccc6)cccc45)nc(-c4cccc5c4oc4ccccc45)n3)cc2)cc1 ; c1ccc(-c2cccc(-c3nc(-c4ccc5c(c4)sc4c(-c6ccccc6)cccc45)nc(-c4cccc5c4oc4ccccc45)n3)c2)cc1]

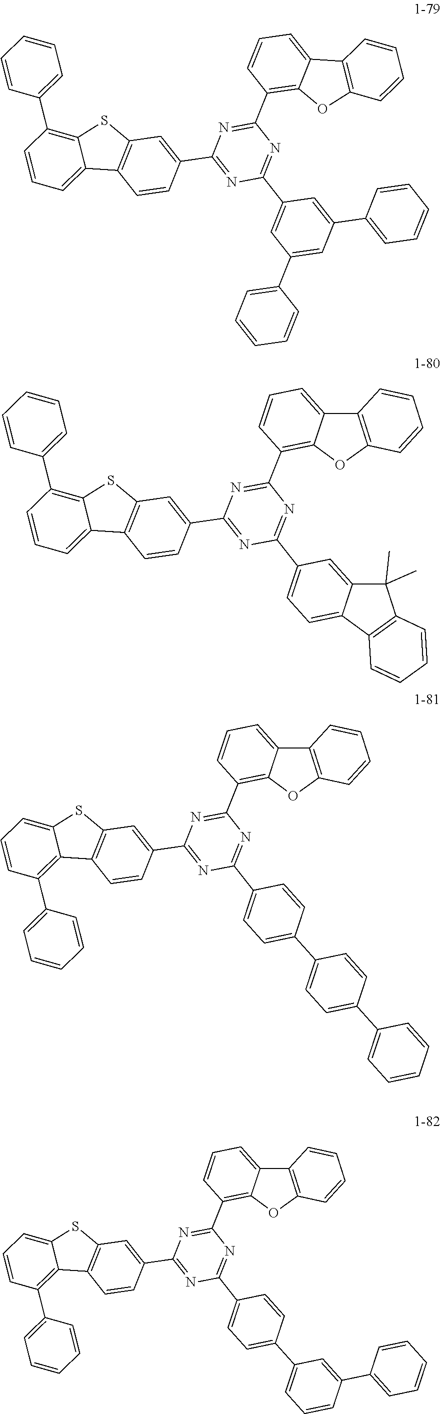 [SMILES: c1ccc(-c2cc(-c3ccccc3)cc(-c3nc(-c4ccc5c(c4)sc4c(-c6ccccc6)cccc45)nc(-c4cccc5c4oc4ccccc45)n3)c2)cc1 ; CC1(C)c2ccccc2-c2ccc(-c3nc(-c4ccc5c(c4)sc4c(-c6ccccc6)cccc45)nc(-c4cccc5c4oc4ccccc45)n3)cc21 ; c1ccc(-c2ccc(-c3ccc(-c4nc(-c5ccc6c(c5)sc5cccc(-c7ccccc7)c56)nc(-c5cccc6c5oc5ccccc56)n4)cc3)cc2)cc1 ; c1ccc(-c2cccc(-c3ccc(-c4nc(-c5ccc6c(c5)sc5cccc(-c7ccccc7)c56)nc(-c5cccc6c5oc5ccccc56)n4)cc3)c2)cc1]

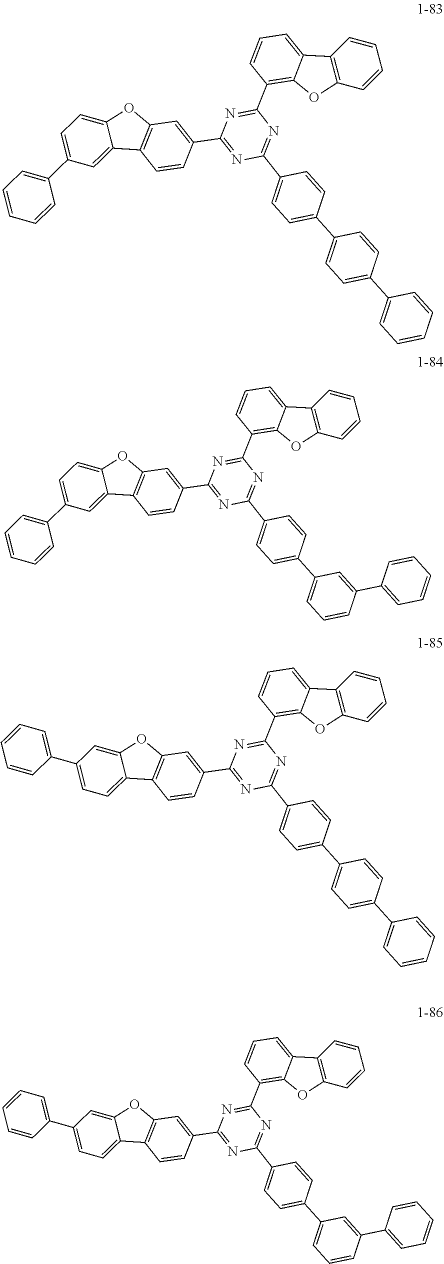 [SMILES: c1ccc(-c2ccc(-c3ccc(-c4nc(-c5ccc6c(c5)oc5ccc(-c7ccccc7)cc56)nc(-c5cccc6c5oc5ccccc56)n4)cc3)cc2)cc1 ; c1ccc(-c2cccc(-c3ccc(-c4nc(-c5ccc6c(c5)oc5ccc(-c7ccccc7)cc56)nc(-c5cccc6c5oc5ccccc56)n4)cc3)c2)cc1 ; c1ccc(-c2ccc(-c3ccc(-c4nc(-c5ccc6c(c5)oc5cc(-c7ccccc7)ccc56)nc(-c5cccc6c5oc5ccccc56)n4)cc3)cc2)cc1 ; c1ccc(-c2cccc(-c3ccc(-c4nc(-c5ccc6c(c5)oc5cc(-c7ccccc7)ccc56)nc(-c5cccc6c5oc5ccccc56)n4)cc3)c2)cc1]

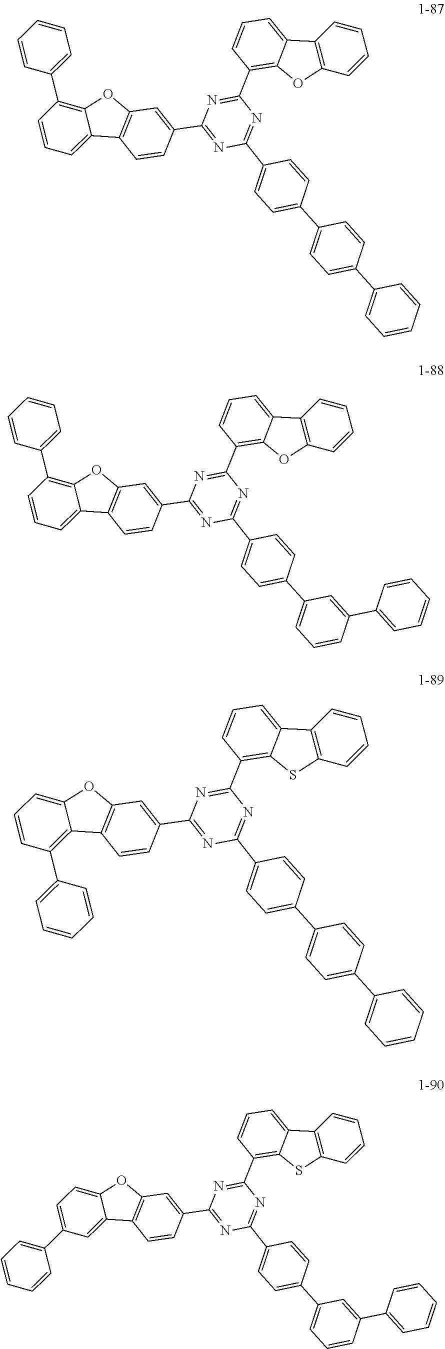 [SMILES: c1ccc(-c2ccc(-c3ccc(-c4nc(-c5ccc6c(c5)oc5c(-c7ccccc7)cccc56)nc(-c5cccc6c5oc5ccccc56)n4)cc3)cc2)cc1 ; c1ccc(-c2cccc(-c3ccc(-c4nc(-c5ccc6c(c5)oc5c(-c7ccccc7)cccc56)nc(-c5cccc6c5oc5ccccc56)n4)cc3)c2)cc1 ; c1ccc(-c2ccc(-c3ccc(-c4nc(-c5ccc6c(c5)oc5cccc(-c7ccccc7)c56)nc(-c5cccc6c5sc5ccccc56)n4)cc3)cc2)cc1 ; c1ccc(-c2cccc(-c3ccc(-c4nc(-c5ccc6c(c5)oc5ccc(-c7ccccc7)cc56)nc(-c5cccc6c5sc5ccccc56)n4)cc3)c2)cc1]

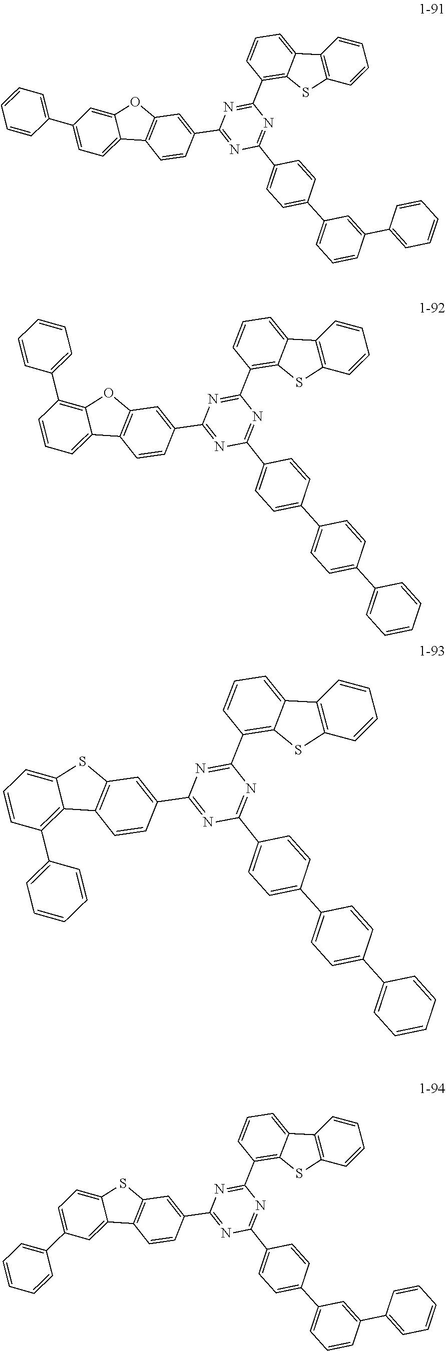 [SMILES: c1ccc(-c2cccc(-c3ccc(-c4nc(-c5ccc6c(c5)oc5cc(-c7ccccc7)ccc56)nc(-c5cccc6c5sc5ccccc56)n4)cc3)c2)cc1 ; c1ccc(-c2ccc(-c3ccc(-c4nc(-c5ccc6c(c5)oc5c(-c7ccccc7)cccc56)nc(-c5cccc6c5sc5ccccc56)n4)cc3)cc2)cc1 ; c1ccc(-c2ccc(-c3ccc(-c4nc(-c5ccc6c(c5)sc5cccc(-c7ccccc7)c56)nc(-c5cccc6c5sc5ccccc56)n4)cc3)cc2)cc1 ; c1ccc(-c2cccc(-c3ccc(-c4nc(-c5ccc6c(c5)sc5ccc(-c7ccccc7)cc56)nc(-c5cccc6c5sc5ccccc56)n4)cc3)c2)cc1]

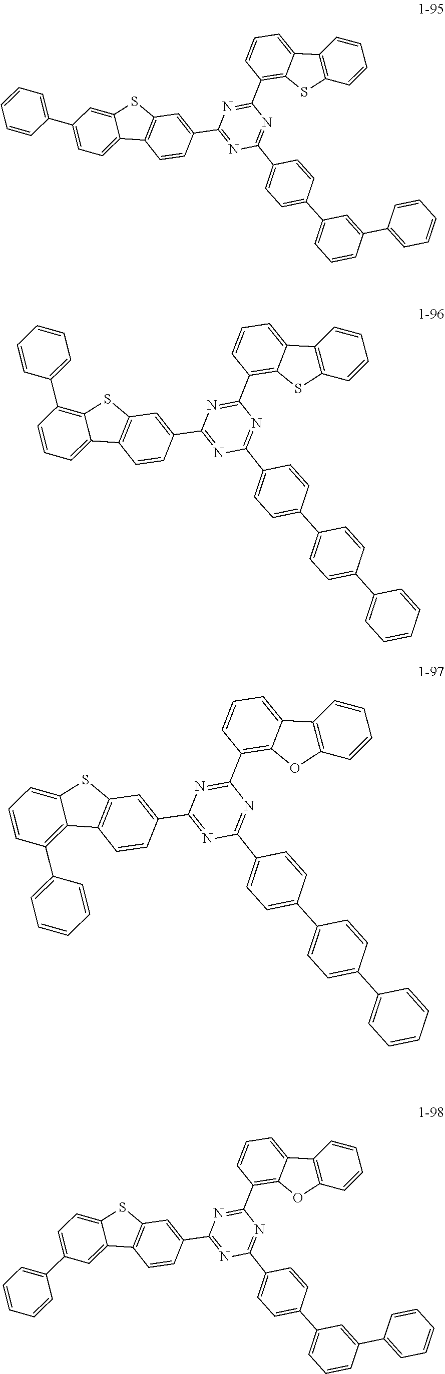 [SMILES: c1ccc(-c2cccc(-c3ccc(-c4nc(-c5ccc6c(c5)sc5cc(-c7ccccc7)ccc56)nc(-c5cccc6c5sc5ccccc56)n4)cc3)c2)cc1 ; c1ccc(-c2ccc(-c3ccc(-c4nc(-c5ccc6c(c5)sc5c(-c7ccccc7)cccc56)nc(-c5cccc6c5sc5ccccc56)n4)cc3)cc2)cc1 ; c1ccc(-c2ccc(-c3ccc(-c4nc(-c5ccc6c(c5)sc5cccc(-c7ccccc7)c56)nc(-c5cccc6c5oc5ccccc56)n4)cc3)cc2)cc1 ; c1ccc(-c2cccc(-c3ccc(-c4nc(-c5ccc6c(c5)sc5ccc(-c7ccccc7)cc56)nc(-c5cccc6c5oc5ccccc56)n4)cc3)c2)cc1]

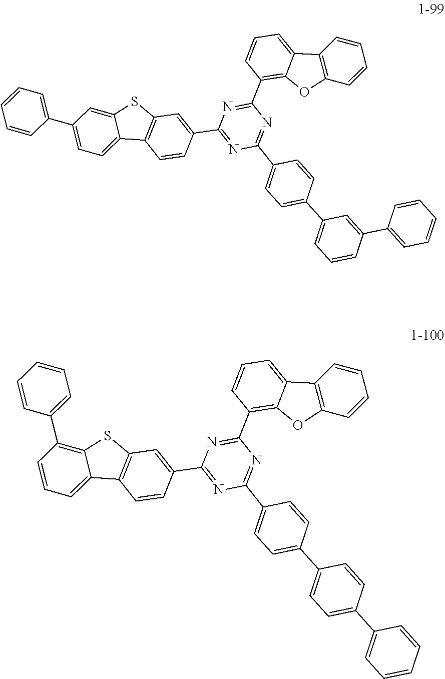 [SMILES: c1ccc(-c2cccc(-c3ccc(-c4nc(-c5ccc6c(c5)sc5cc(-c7ccccc7)ccc56)nc(-c5cccc6c5oc5ccccc56)n4)cc3)c2)cc1 ; c1ccc(-c2ccc(-c3ccc(-c4nc(-c5ccc6c(c5)sc5c(-c7ccccc7)cccc56)nc(-c5cccc6c5oc5ccccc56)n4)cc3)cc2)cc1]

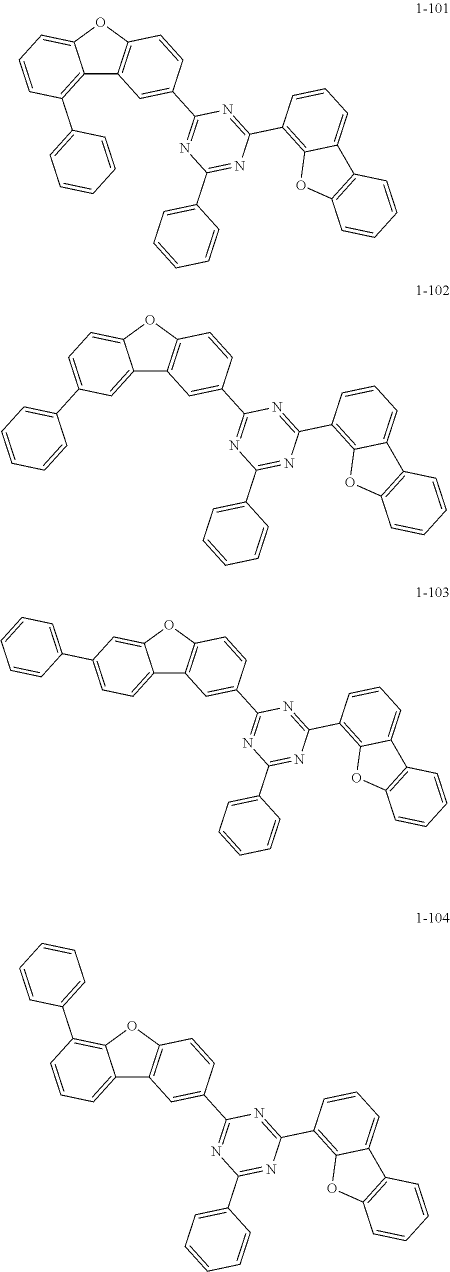 [SMILES: c1ccc(-c2nc(-c3ccc4oc5cccc(-c6ccccc6)c5c4c3)nc(-c3cccc4c3oc3ccccc34)n2)cc1 ; c1ccc(-c2ccc3oc4ccc(-c5nc(-c6ccccc6)nc(-c6cccc7c6oc6ccccc67)n5)cc4c3c2)cc1 ; c1ccc(-c2ccc3c(c2)oc2ccc(-c4nc(-c5ccccc5)nc(-c5cccc6c5oc5ccccc56)n4)cc23)cc1 ; c1ccc(-c2nc(-c3ccc4oc5c(-c6ccccc6)cccc5c4c3)nc(-c3cccc4c3oc3ccccc34)n2)cc1]

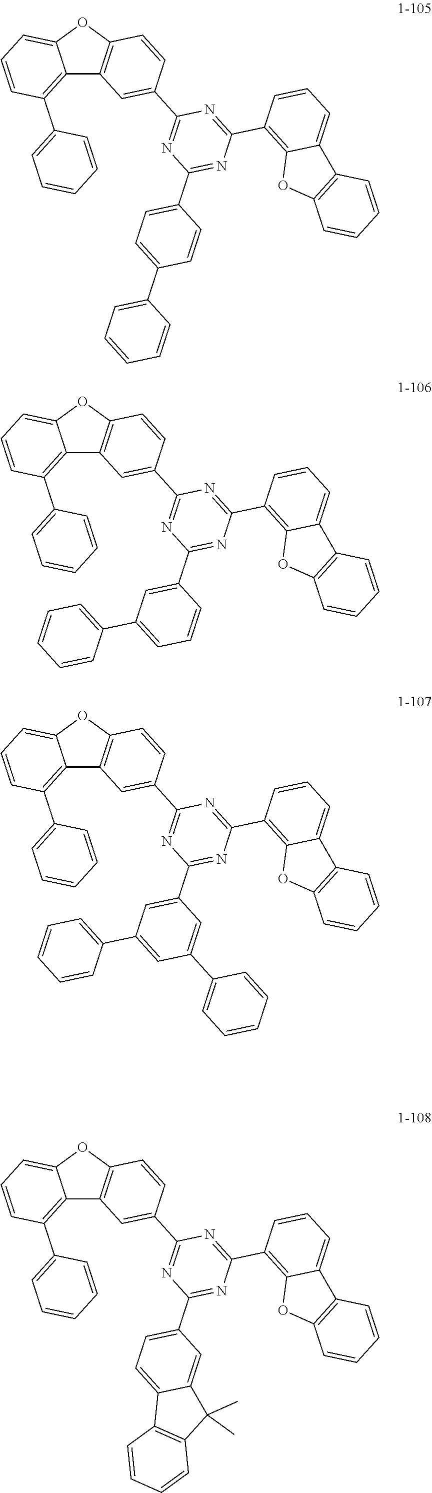

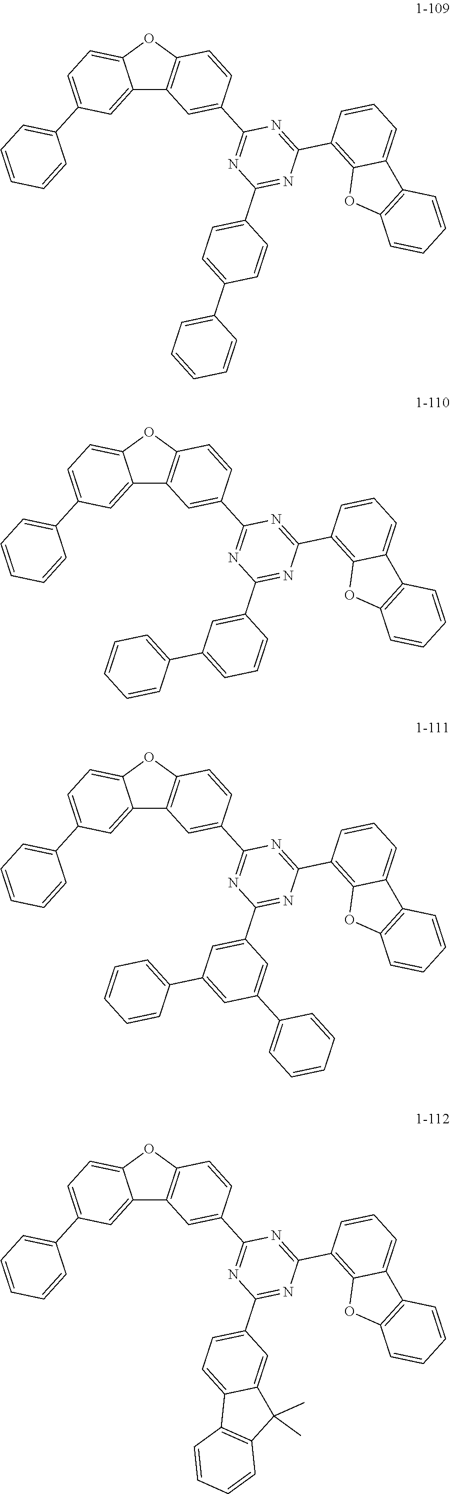

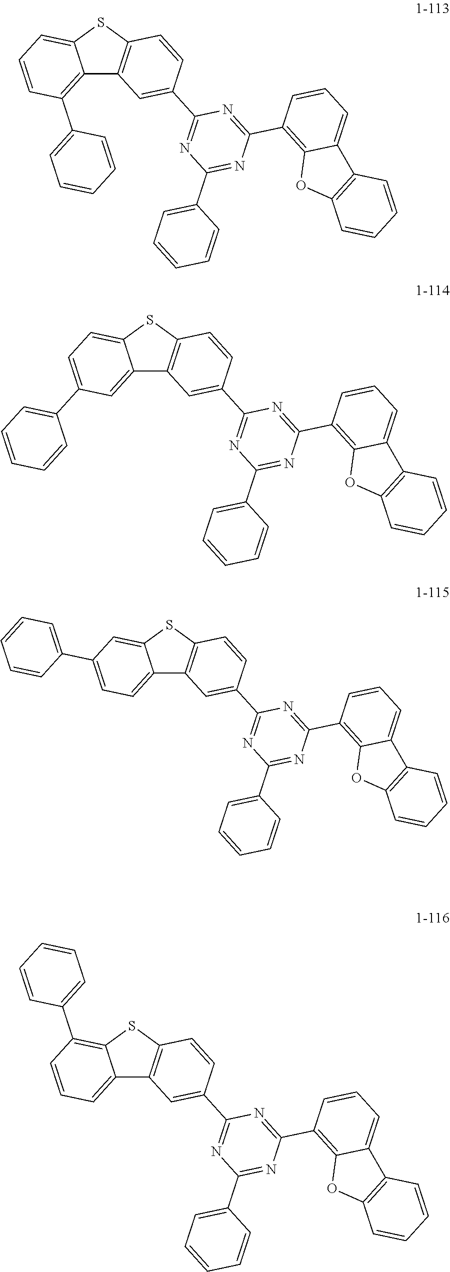 [SMILES: c1ccc(-c2nc(-c3ccc4sc5cccc(-c6ccccc6)c5c4c3)nc(-c3cccc4c3oc3ccccc34)n2)cc1 ; c1ccc(-c2ccc3sc4ccc(-c5nc(-c6ccccc6)nc(-c6cccc7c6oc6ccccc67)n5)cc4c3c2)cc1 ; c1ccc(-c2ccc3c(c2)sc2ccc(-c4nc(-c5ccccc5)nc(-c5cccc6c5oc5ccccc56)n4)cc23)cc1 ; c1ccc(-c2nc(-c3ccc4sc5c(-c6ccccc6)cccc5c4c3)nc(-c3cccc4c3oc3ccccc34)n2)cc1]

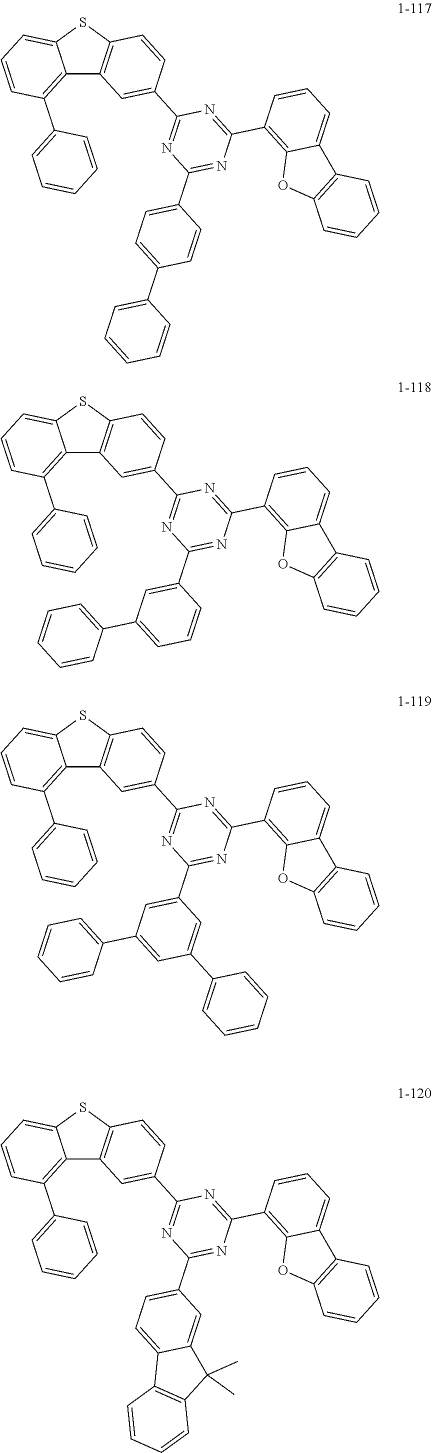

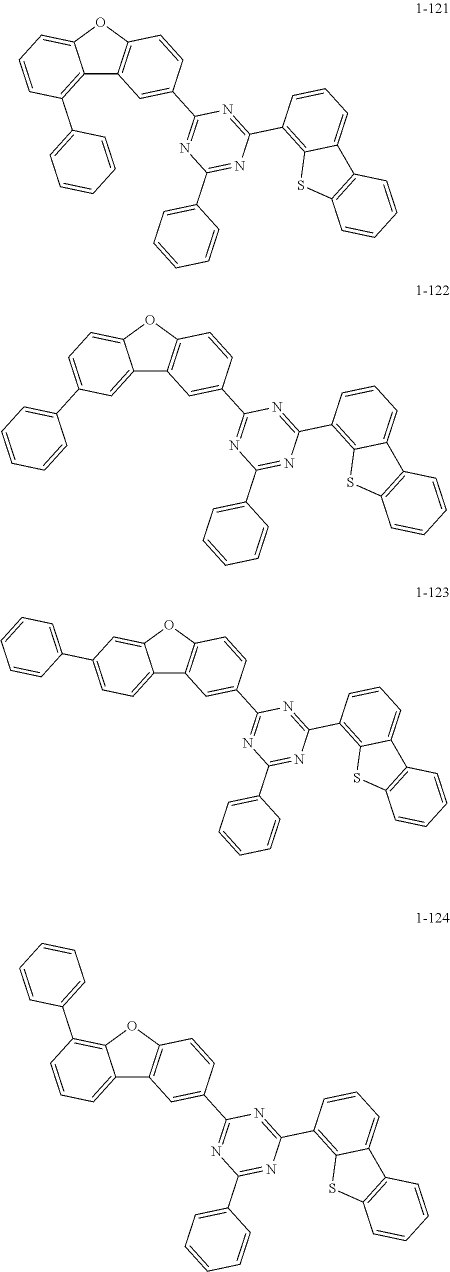 [SMILES: c1ccc(-c2nc(-c3ccc4oc5cccc(-c6ccccc6)c5c4c3)nc(-c3cccc4c3sc3ccccc34)n2)cc1 ; c1ccc(-c2ccc3oc4ccc(-c5nc(-c6ccccc6)nc(-c6cccc7c6sc6ccccc67)n5)cc4c3c2)cc1 ; c1ccc(-c2ccc3c(c2)oc2ccc(-c4nc(-c5ccccc5)nc(-c5cccc6c5sc5ccccc56)n4)cc23)cc1 ; c1ccc(-c2nc(-c3ccc4oc5c(-c6ccccc6)cccc5c4c3)nc(-c3cccc4c3sc3ccccc34)n2)cc1]

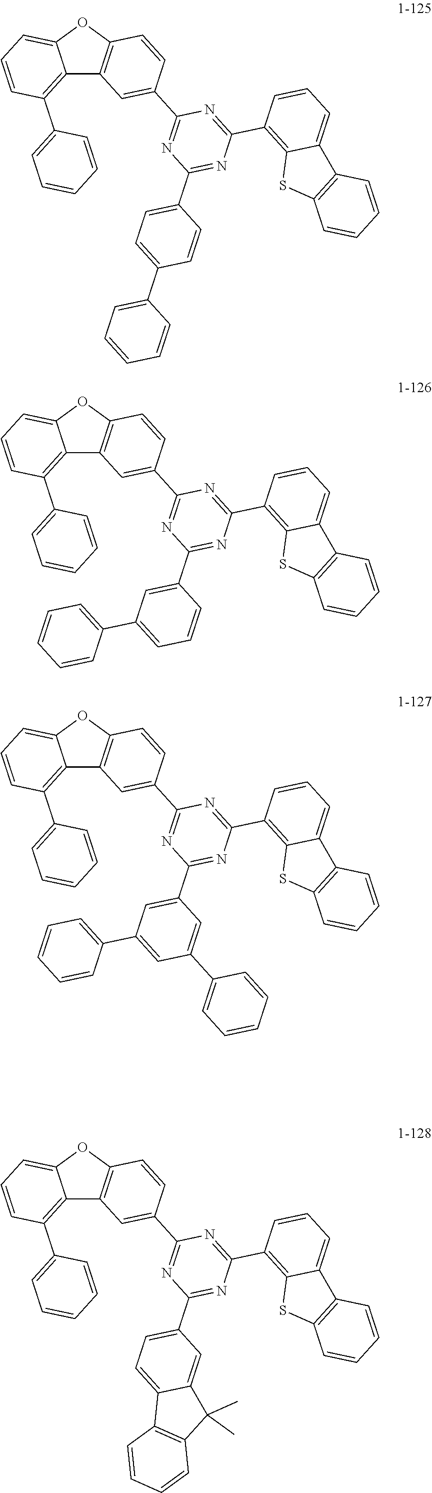

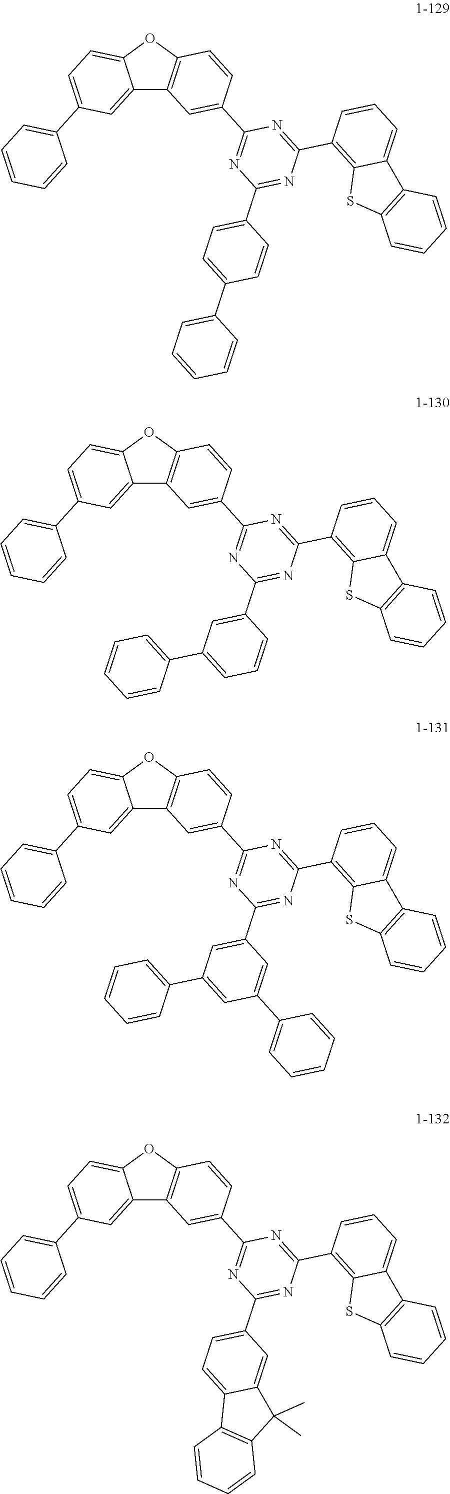

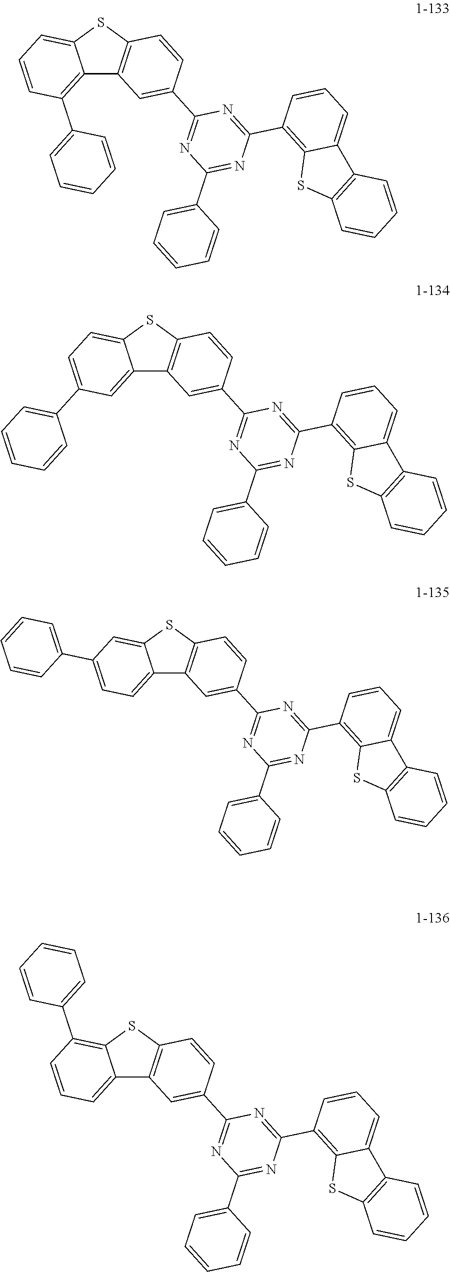 [SMILES: c1ccc(-c2nc(-c3ccc4sc5cccc(-c6ccccc6)c5c4c3)nc(-c3cccc4c3sc3ccccc34)n2)cc1 ; c1ccc(-c2ccc3sc4ccc(-c5nc(-c6ccccc6)nc(-c6cccc7c6sc6ccccc67)n5)cc4c3c2)cc1 ; c1ccc(-c2ccc3c(c2)sc2ccc(-c4nc(-c5ccccc5)nc(-c5cccc6c5sc5ccccc56)n4)cc23)cc1 ; c1ccc(-c2nc(-c3ccc4sc5c(-c6ccccc6)cccc5c4c3)nc(-c3cccc4c3sc3ccccc34)n2)cc1]

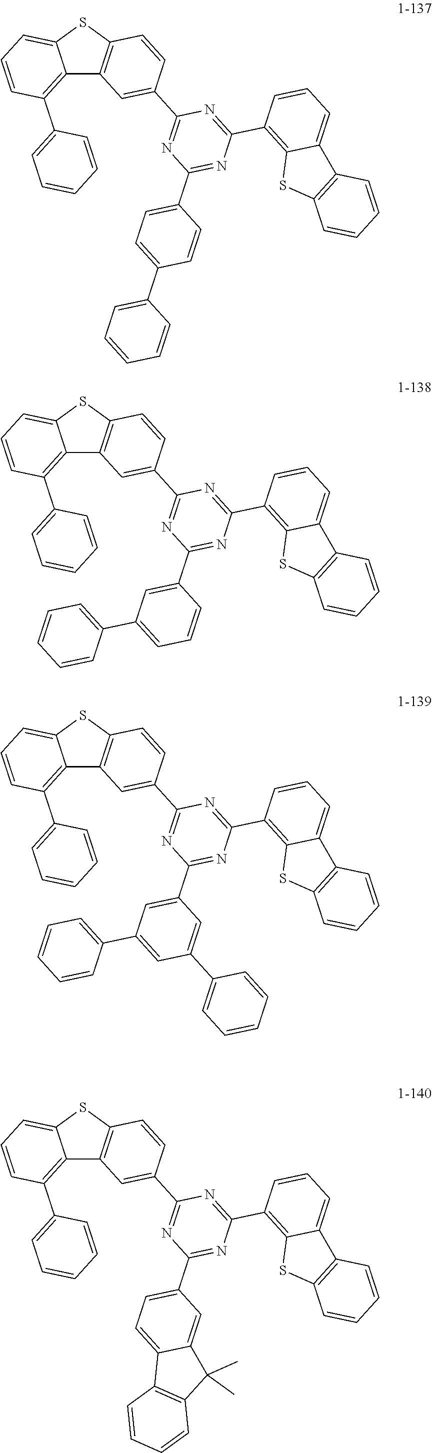

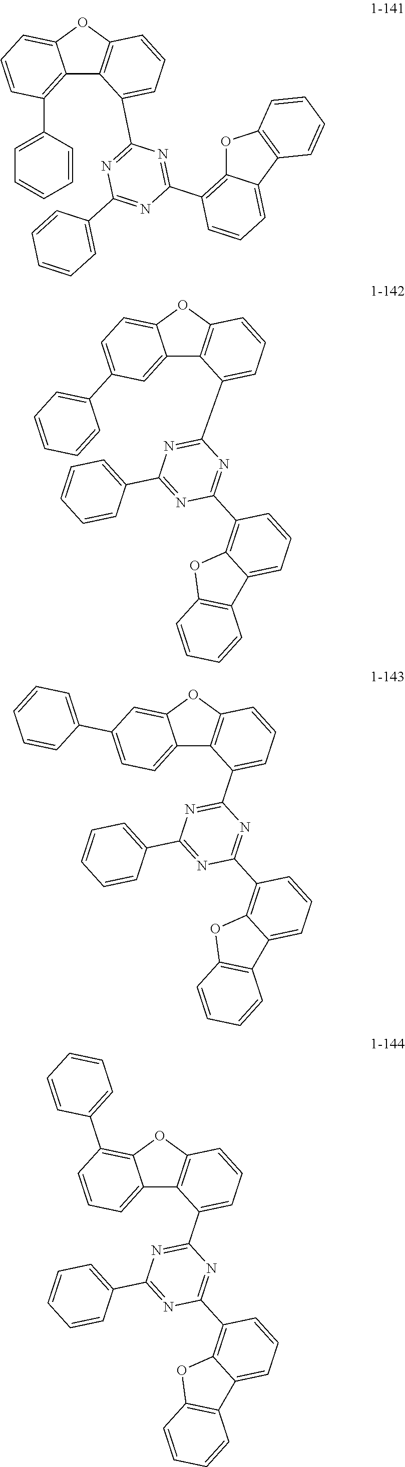

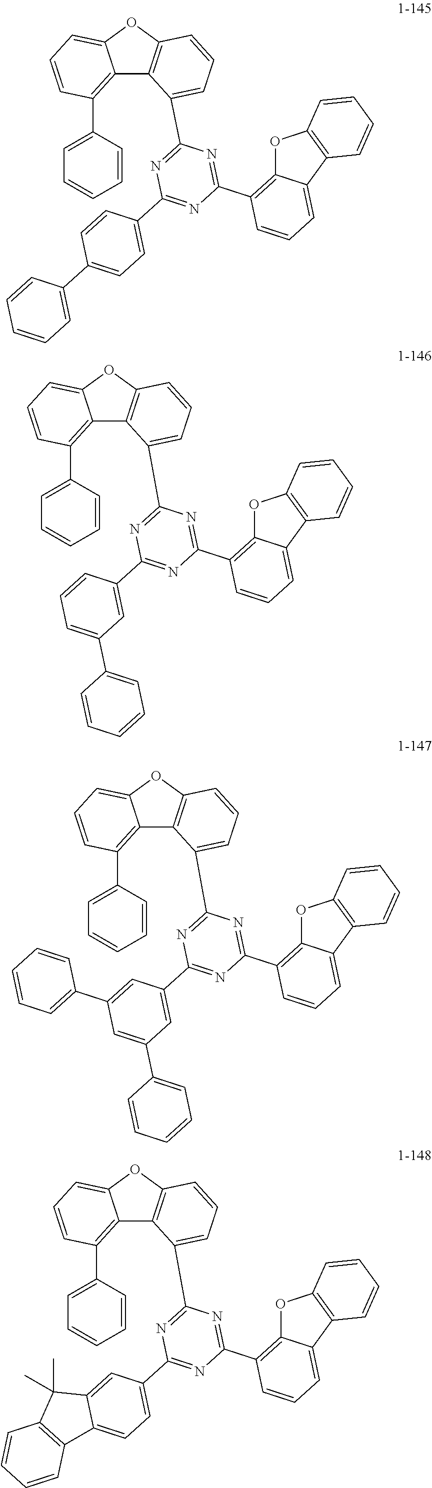

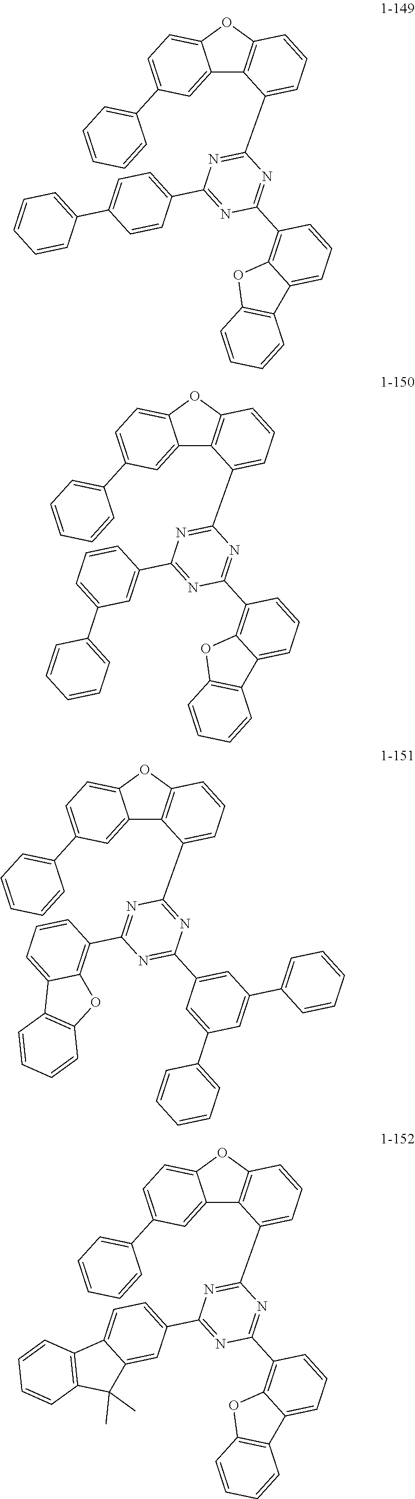

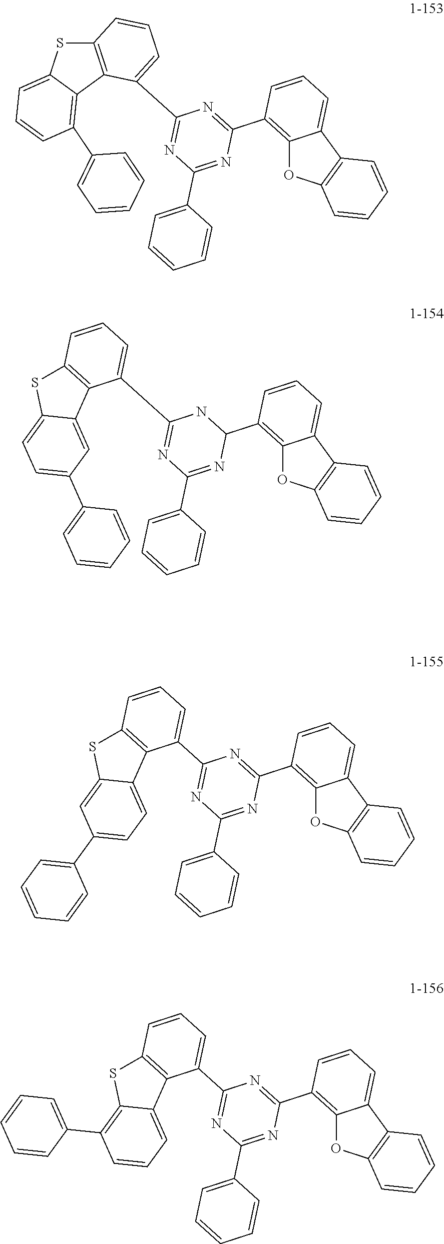

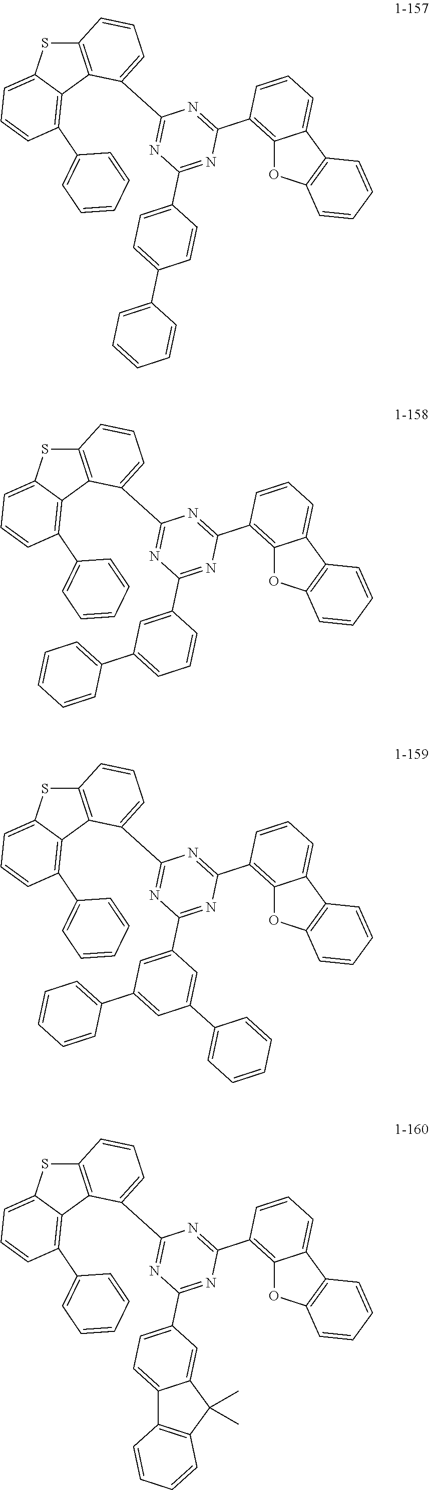 [SMILES: c1ccc(-c2ccc(-c3nc(-c4cccc5c4oc4ccccc45)nc(-c4cccc5sc6cccc(-c7ccccc7)c6c45)n3)cc2)cc1 ; c1ccc(-c2cccc(-c3nc(-c4cccc5c4oc4ccccc45)nc(-c4cccc5sc6cccc(-c7ccccc7)c6c45)n3)c2)cc1 ; c1ccc(-c2cc(-c3ccccc3)cc(-c3nc(-c4cccc5c4oc4ccccc45)nc(-c4cccc5sc6cccc(-c7ccccc7)c6c45)n3)c2)cc1 ; CC1(C)c2ccccc2-c2ccc(-c3nc(-c4cccc5c4oc4ccccc45)nc(-c4cccc5sc6cccc(-c7ccccc7)c6c45)n3)cc21]

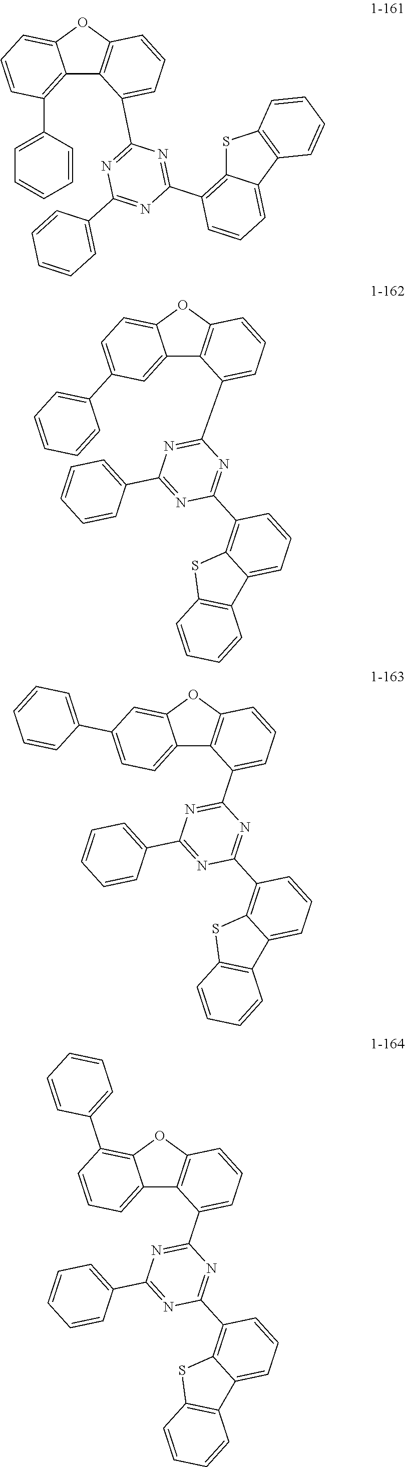

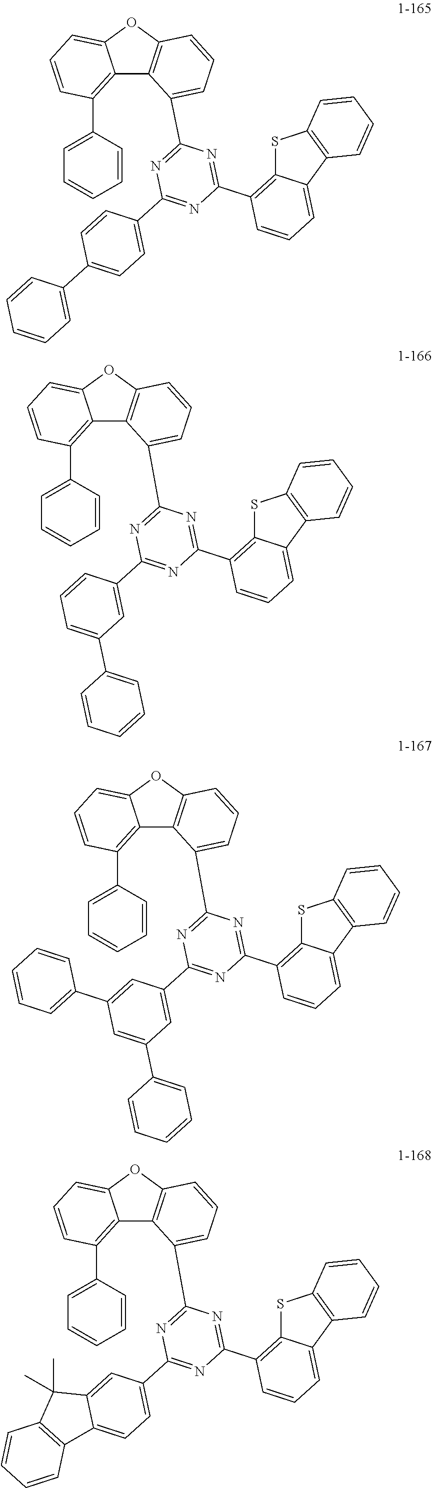

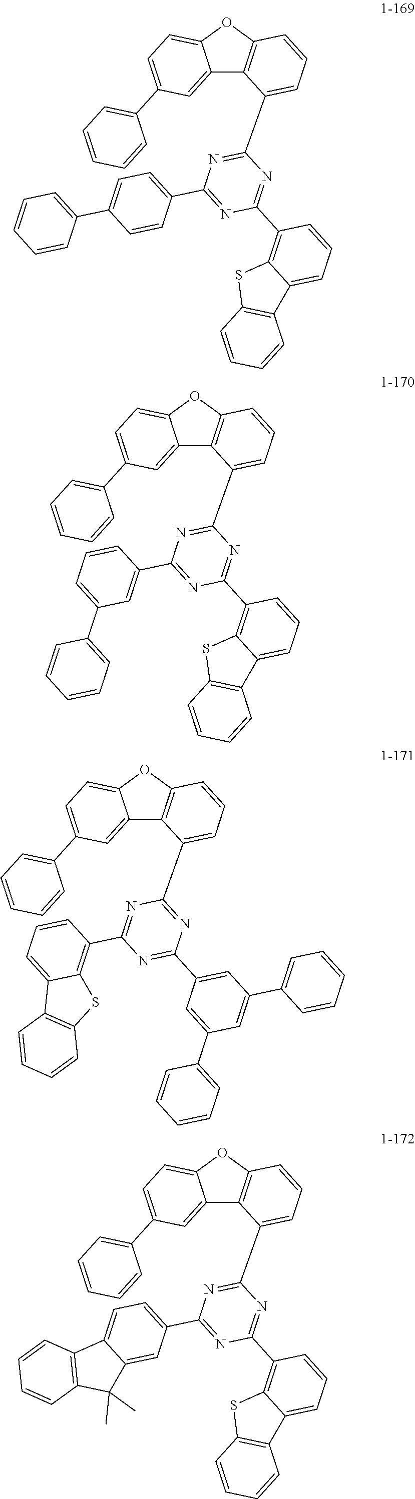

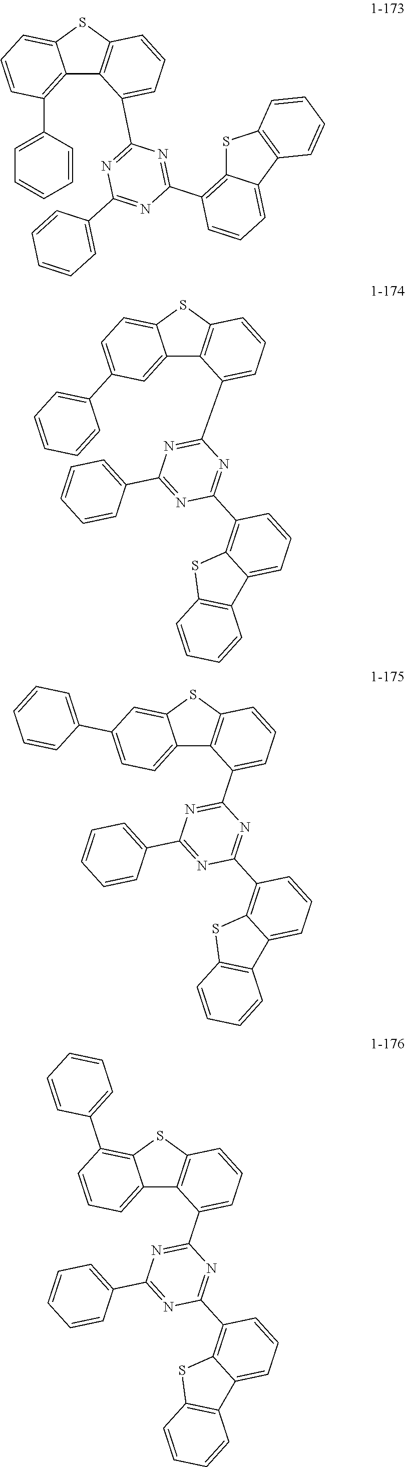

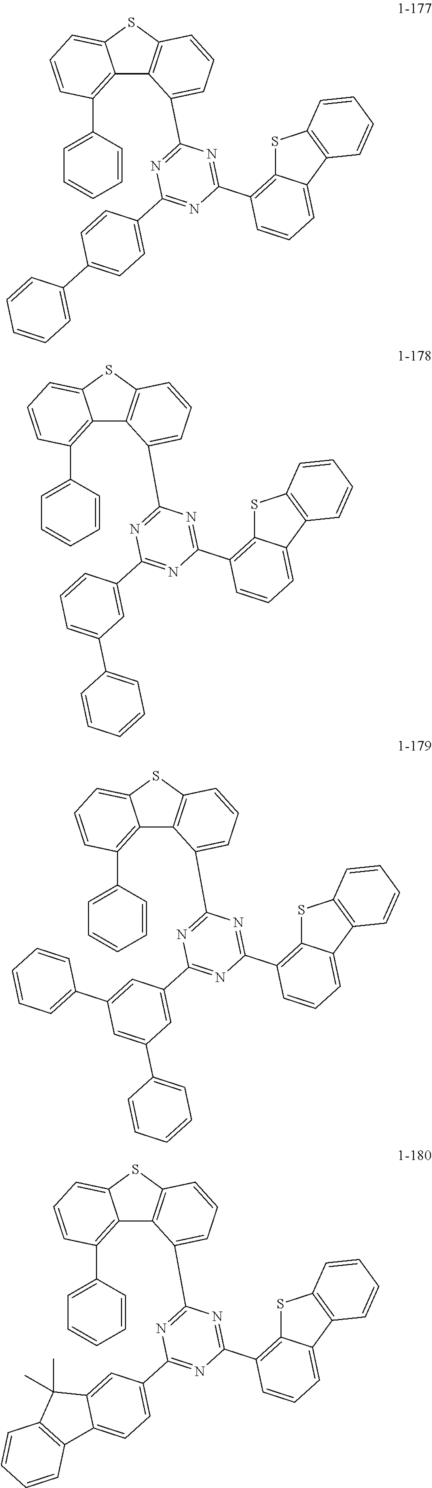

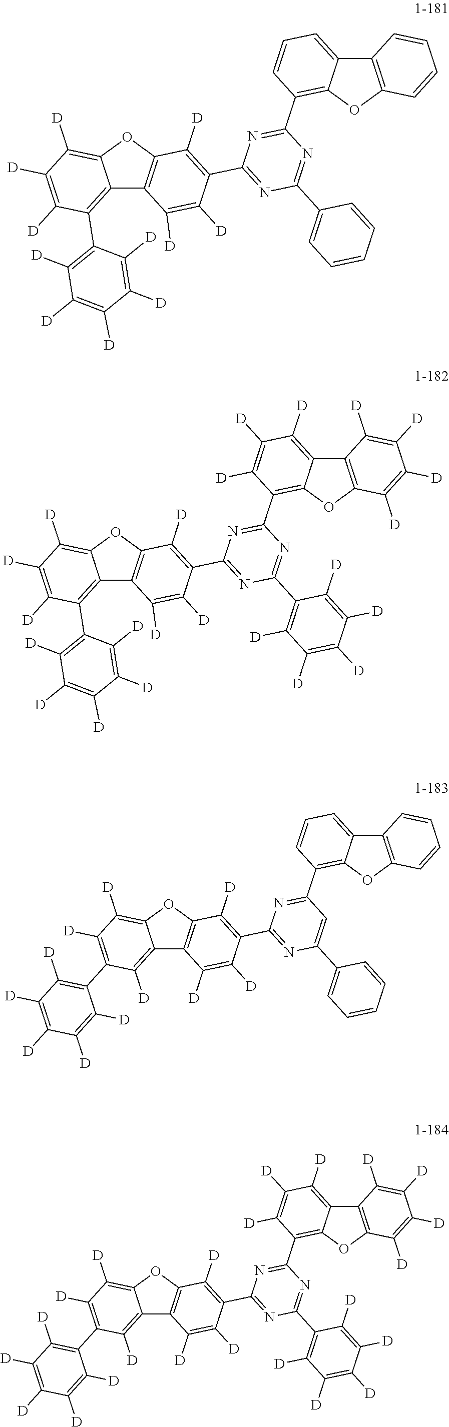

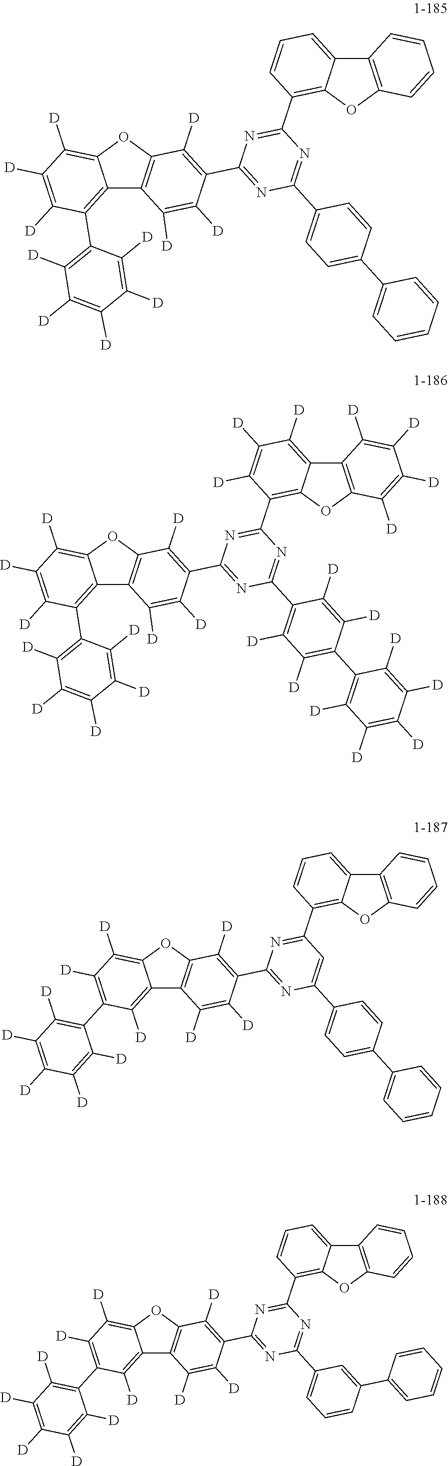

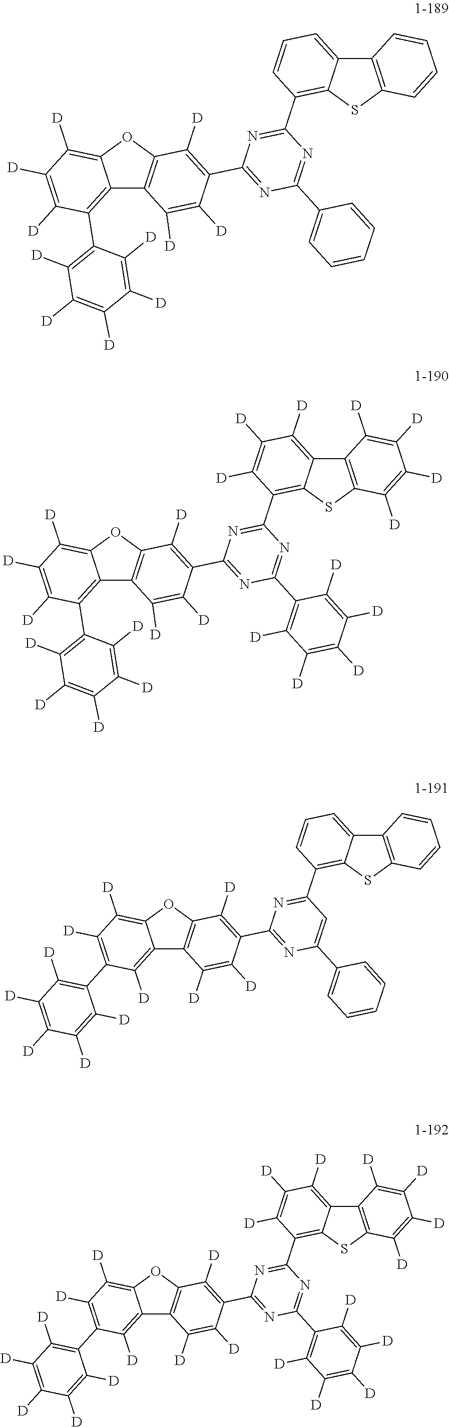

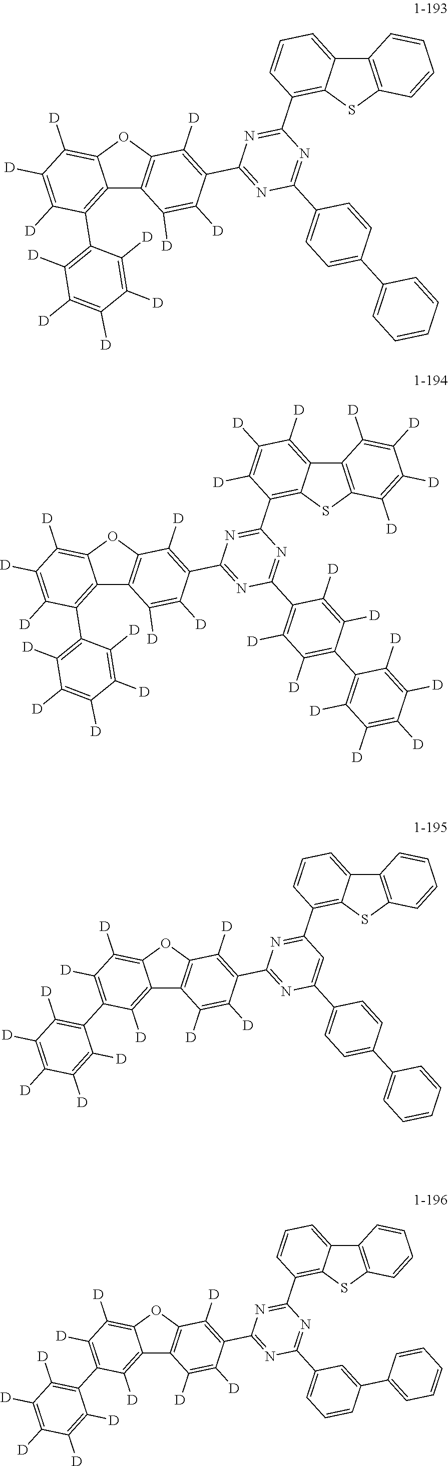

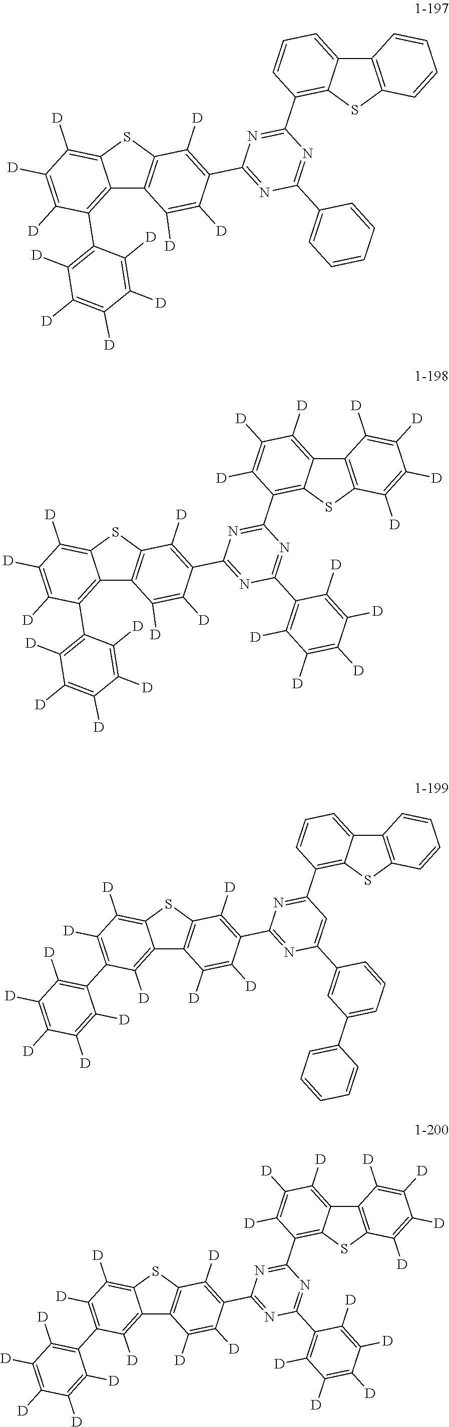

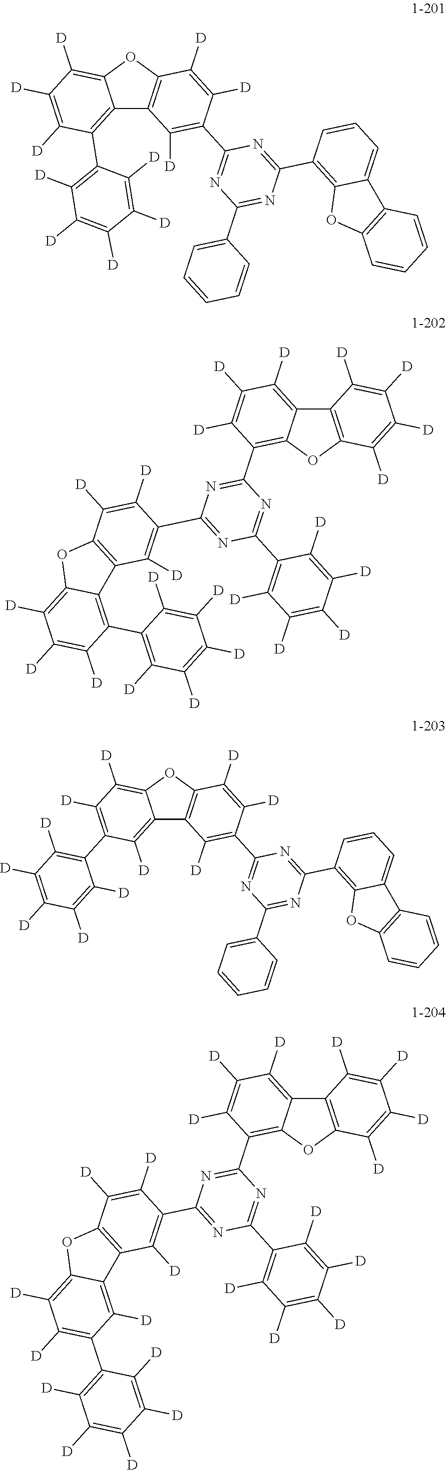

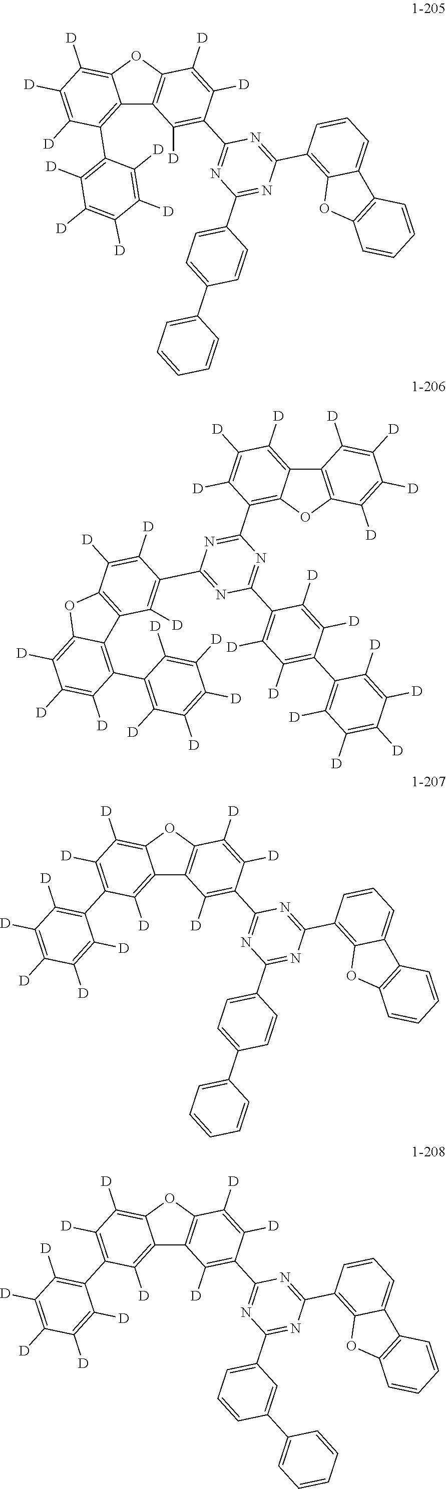

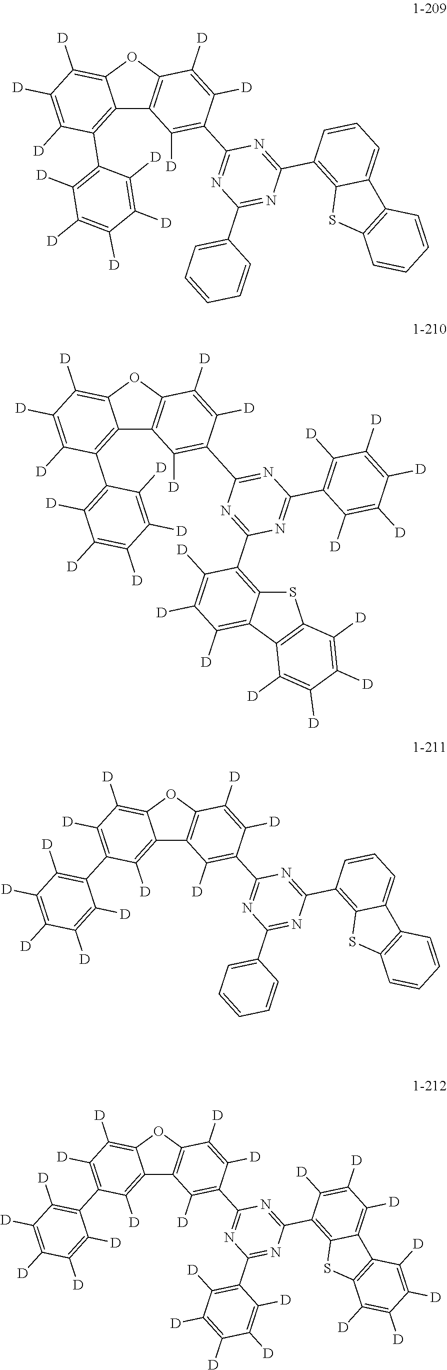

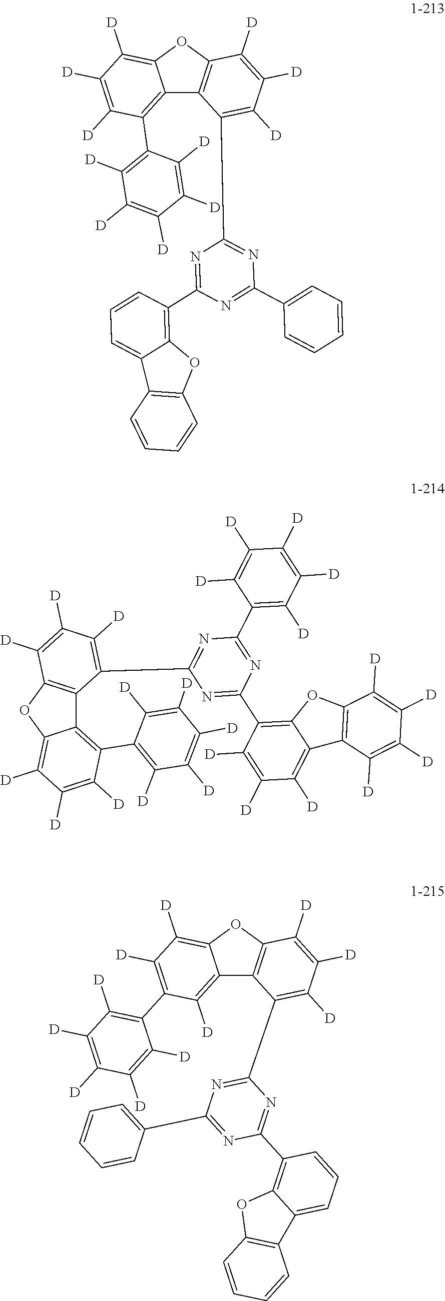

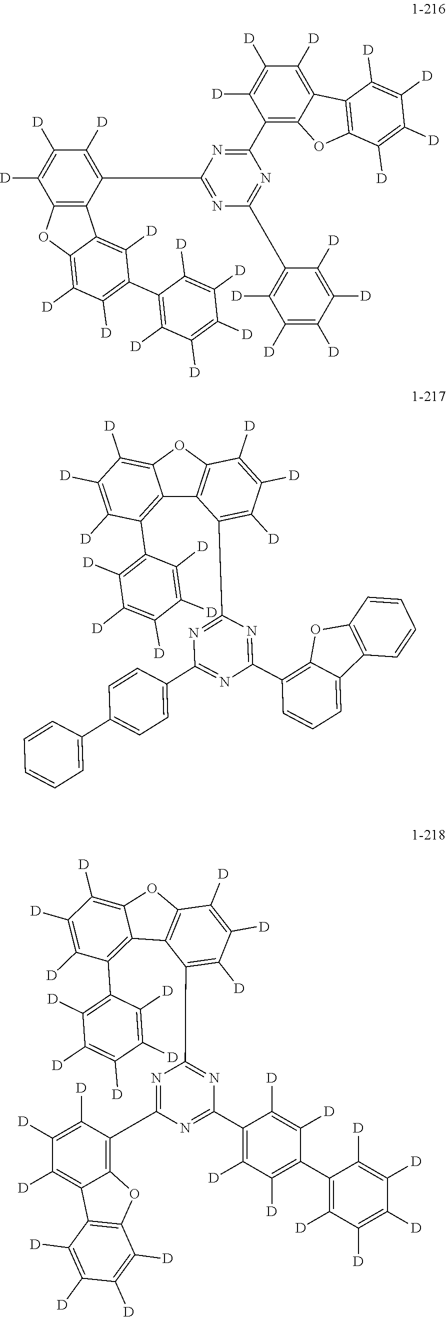

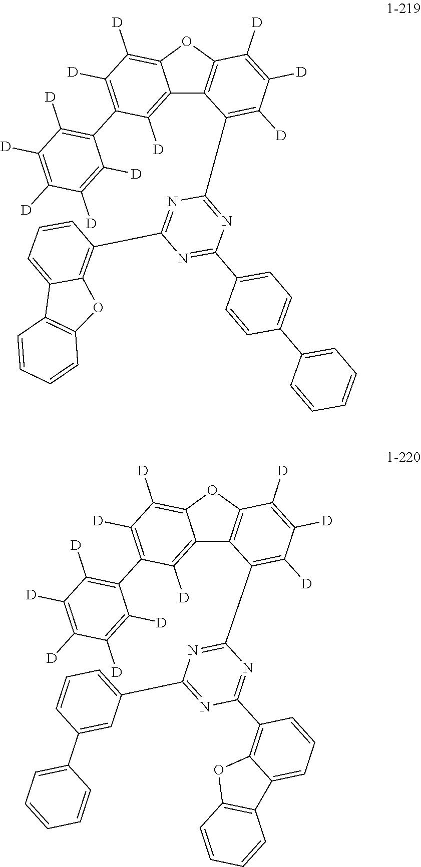 [SMILES: [2H]c1c([2H])c([2H])c(-c2c([2H])c([2H])c3oc4c([2H])c([2H])c([2H])c(-c5nc(-c6ccc(-c7ccccc7)cc6)nc(-c6cccc7c6oc6ccccc67)n5)c4c3c2[2H])c([2H])c1[2H] ; [2H]c1c([2H])c([2H])c(-c2c([2H])c([2H])c3oc4c([2H])c([2H])c([2H])c(-c5nc(-c6cccc(-c7ccccc7)c6)nc(-c6cccc7c6oc6ccccc67)n5)c4c3c2[2H])c([2H])c1[2H]]

Further, various substituents may be introduced into the structure of Chemical Formula 1 to synthesize a compound having inherent characteristics of a substituent introduced. For example, a substituent usually used for a hole injection material, a hole transport material, a light emitting material, an electron transport material and an electron injection material, which are used when manufacturing an organic light emitting device, may be introduced into the core structure to synthesize a material which satisfies conditions required for each organic material layer.

In addition, by introducing various substituents into the structure of Chemical Formula 1 or changing the binding position, the bandgap may be finely adjusted, and meanwhile, the characteristics at the interface between the organic material layers may be improved.

In addition, the compound of Chemical Formula 1 has excellent thermal stability, and such thermal stability provides driving stability to the organic light emitting device and improves service life characteristics.

In an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In another exemplary embodiment, provided is an organic light emitting device including: a first electrode; a second electrode provided to face the first electrode; and an organic material layer having one or more layers provided between the first electrode and the second electrode, in which one or more layers of the organic material layer include one heterocyclic compound represented by Chemical Formula 1.

In still another exemplary embodiment, provided is an organic light emitting device including: a first electrode; a second electrode provided to face the first electrode; and an organic material layer having one or more layers provided between the first electrode and the second electrode, in which one or more layers of the organic material layer include two or more of the heterocyclic compound represented by Chemical Formula 1.

In yet another exemplary embodiment, the heterocyclic compound represented by Chemical Formula 1 can be used as a light emitting material for a light emitting layer of the organic light emitting device.

In yet another exemplary embodiment, the heterocyclic compound represented by Chemical Formula 1 may be used as a light emitting material for a light emitting layer of an organic light emitting device, and may be used as an n-type host material.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the heterocyclic compound according to Chemical Formula 1 may be used as a material for the blue organic light emitting device.

In an exemplary embodiment of the present application, the organic light emitting device may be a green organic light emitting device, and the heterocyclic compound represented by Chemical Formula 1 may be used as a material for the green organic light emitting device.

In an exemplary embodiment of the present application, the organic light emitting device may be a red organic light emitting device, and the heterocyclic compound represented by Chemical Formula 1 may be used as a material for the red organic light emitting device.

In an exemplary embodiment of the present application, the organic light emitting device may be a blue organic light emitting device, and the heterocyclic compound according to Chemical Formula 1 may be used as a material for a light emitting layer of the blue organic light emitting device.

In an exemplary embodiment of the present application, the organic light emitting device may be a green organic light emitting device, and the heterocyclic compound represented by Chemical Formula 1 may be used as a material for a light emitting layer of the green organic light emitting device.

In an exemplary embodiment of the present application, the organic light emitting device may be a red organic light emitting device, and the heterocyclic compound represented by Chemical Formula 1 may be used as a material for a light emitting layer of the red organic light emitting device.

In an exemplary embodiment of the present application, the specific content on the heterocyclic compound represented by Chemical Formula 1 is the same as that described above.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present application, as the iridium-based dopant, Ir(ppy)₃ as a green phosphorescent dopant may be used.

In an exemplary embodiment of the present application, provided is an organic light emitting device, in which the organic material layer of the organic light emitting device includes a light emitting layer, and the light emitting layer includes the heterocyclic compound.

In an exemplary embodiment of the present application, provided is an organic light emitting device, in which the organic material layer of the organic light emitting device includes a light emitting layer, and the light emitting layer includes a host material, and the host material includes the heterocyclic compound.

In the organic light emitting device of the present invention, the organic material layer includes an electron injection layer or an electron transport layer, and the electron injection layer or electron transport layer may include the heterocyclic compound.

In another organic light emitting device, the organic material layer includes an electron blocking layer or a hole blocking layer, and the electron blocking layer or the hole blocking layer may include the heterocyclic compound.

In still another organic light emitting device, the organic material layer includes an electron transport layer, a light emitting layer or a hole blocking layer, and the electron transport layer, the light emitting layer or the hole blocking layer may include the heterocyclic compound.

In the organic light emitting device of the present application, as a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited or used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

The organic light emitting device of the present invention may further include one or two or more layers selected from the group consisting of a light emitting layer, a hole injection layer, a hole transport layer, an electron blocking layer, a hole blocking layer, an electron transport layer and an electron injection layer.

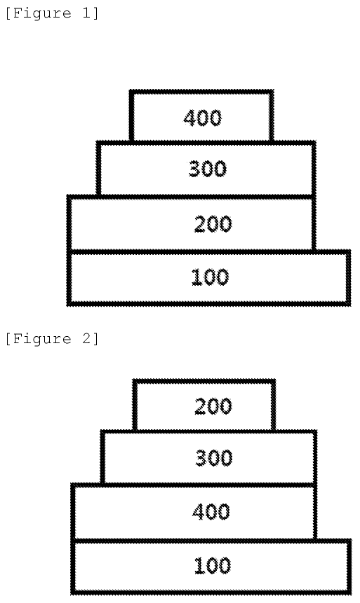

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

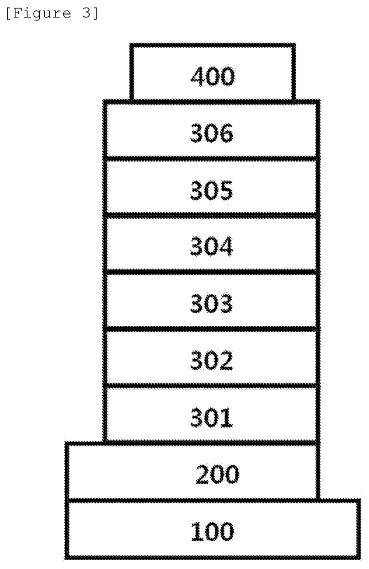

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

In an exemplary embodiment of the present application, provided is an organic light emitting device in which the organic material layer of the organic light emitting device including the heterocyclic compound represented by Chemical Formula 1 further includes a heterocyclic compound represented by the following Chemical Formula 2.

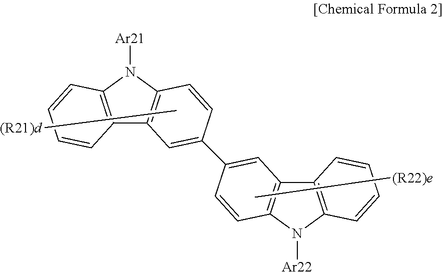

In Chemical Formula 2,
Ar21 and Ar22 are the same as or different from each other, and are each independently a substituted or unsubstituted C6 to C60 aryl group; or a substituted or unsubstituted C2 to C60 heteroaryl group,
R21 and R22 are the same as or different from each other, and are each independently selected from the group consisting of hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more adjacent groups are bonded to each other to form a substituted or unsubstituted aliphatic or aromatic hydrocarbon ring, and
d and e are an integer from 0 to 7, and when d and e are 2 or higher, substituents in the parenthesis are the same as or different from each other.

In an exemplary embodiment of the present application, the deuterium content of Chemical Formula 2 may be 0% to 100%.

In another exemplary embodiment, the deuterium content of Chemical Formula 2 may be 0%, or 20% to 100%.

In still another exemplary embodiment, the deuterium content of Chemical Formula 2 may be 0%, or 40% to 100%.

In yet another exemplary embodiment, the deuterium content of Chemical Formula 2 may be 0%, or 60% to 100%.

In yet another exemplary embodiment, the deuterium content of Chemical Formula 2 may be 0%, or 80% to 100%.

In yet another exemplary embodiment, the deuterium content of Chemical Formula 2 may be 0%, or 100%.

In an exemplary embodiment of the present application, Ar21 and Ar22 are the same as or different from each other, and may be each independently a substituted or unsubstituted C6 to C40 aryl group; or a substituted or unsubstituted C2 to C40 heteroaryl group.

In another exemplary embodiment, Ar21 and Ar22 are the same as or different from each other, and may be each independently a substituted or unsubstituted C6 to C20 aryl group; or a substituted or unsubstituted C2 to C20 heteroaryl group.

In still another exemplary embodiment, Ar21 and Ar22 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted triphenylenyl group; a substituted or unsubstituted dibenzofuran group; or a substituted or unsubstituted dibenzothiophene group.

In an exemplary embodiment of the present application, R21 and R22 are the same as or different from each other, and may be each independently hydrogen; deuterium; a halogen group; a cyano group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In another exemplary embodiment, R21 and R22 are the same as or different from each other, and may be each independently hydrogen; deuterium; a halogen group; a cyano group; a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In still another exemplary embodiment, R21 and R22 are the same as or different from each other, and may be each independently hydrogen; deuterium.

In an exemplary embodiment of the present application, provided is an organic light emitting device in which Chemical Formula 2 is represented by any one of the following compounds.

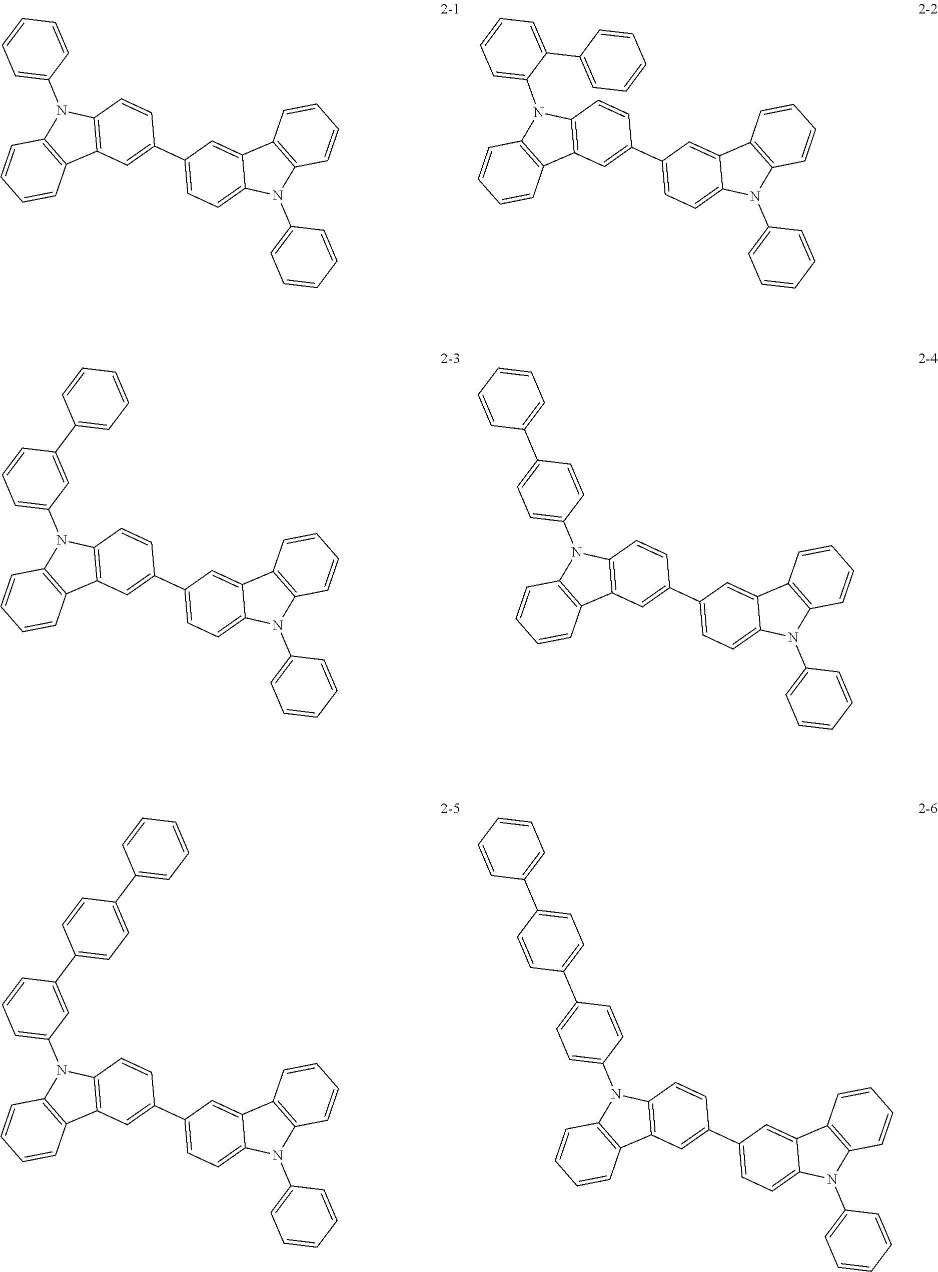 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

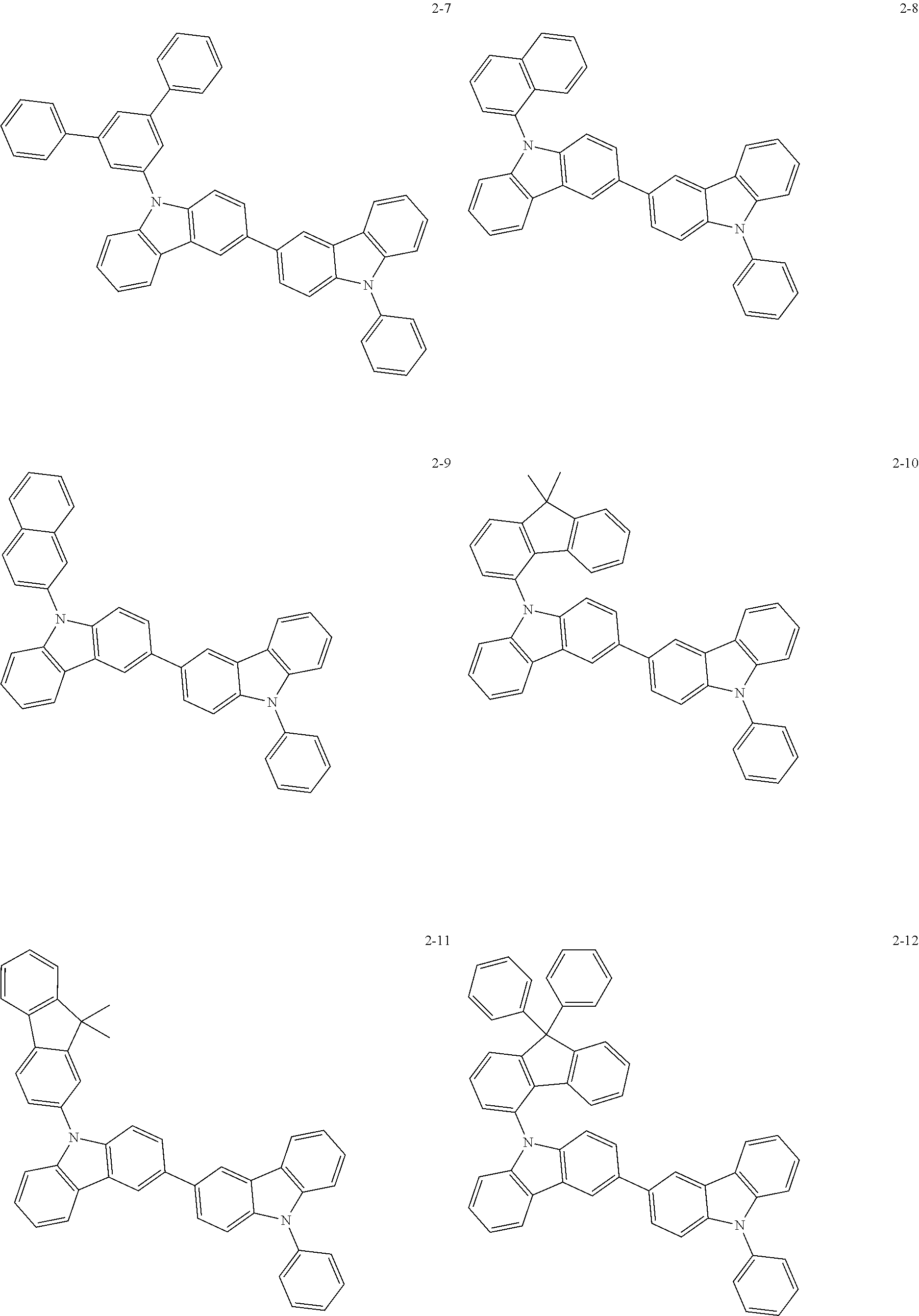

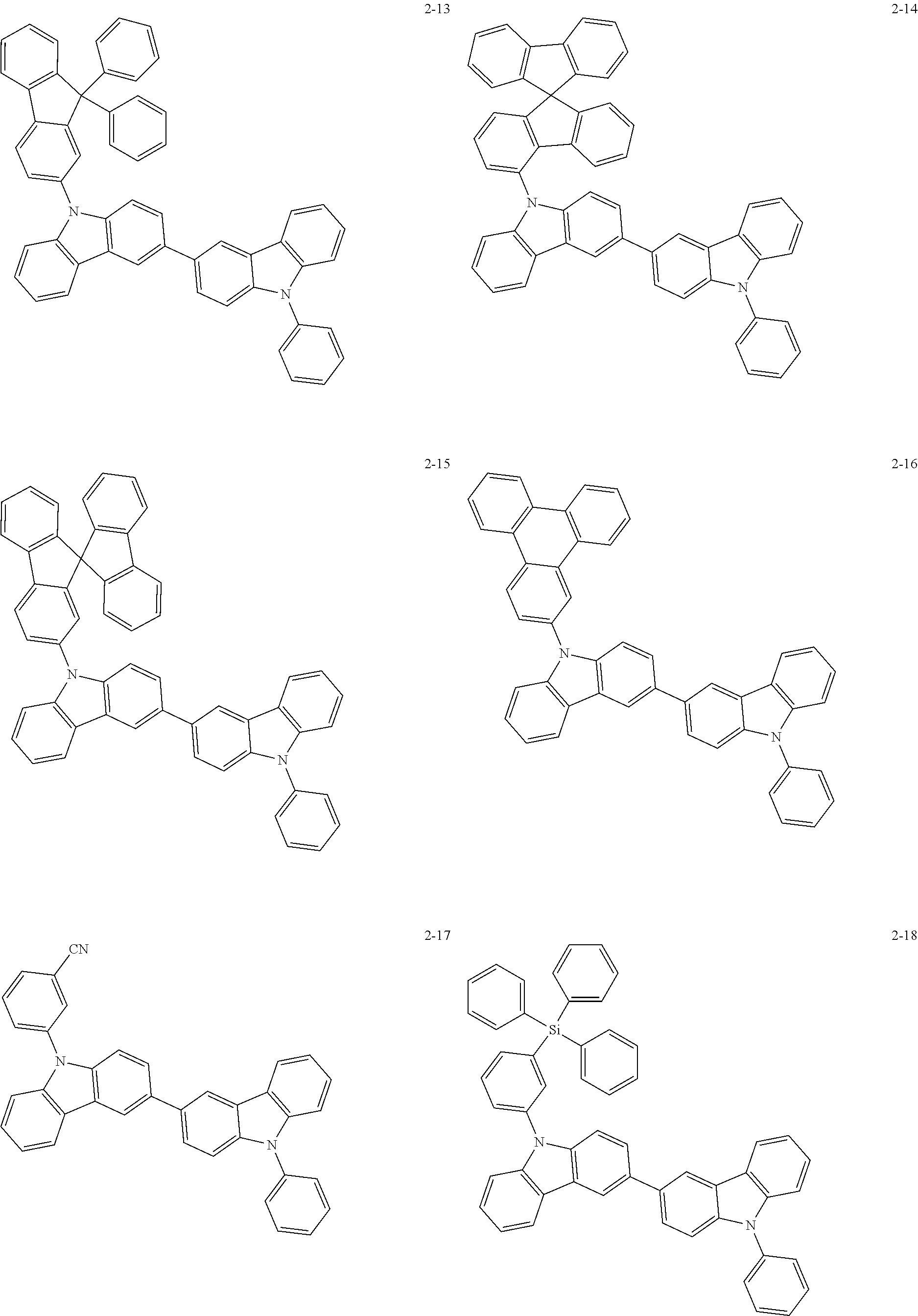

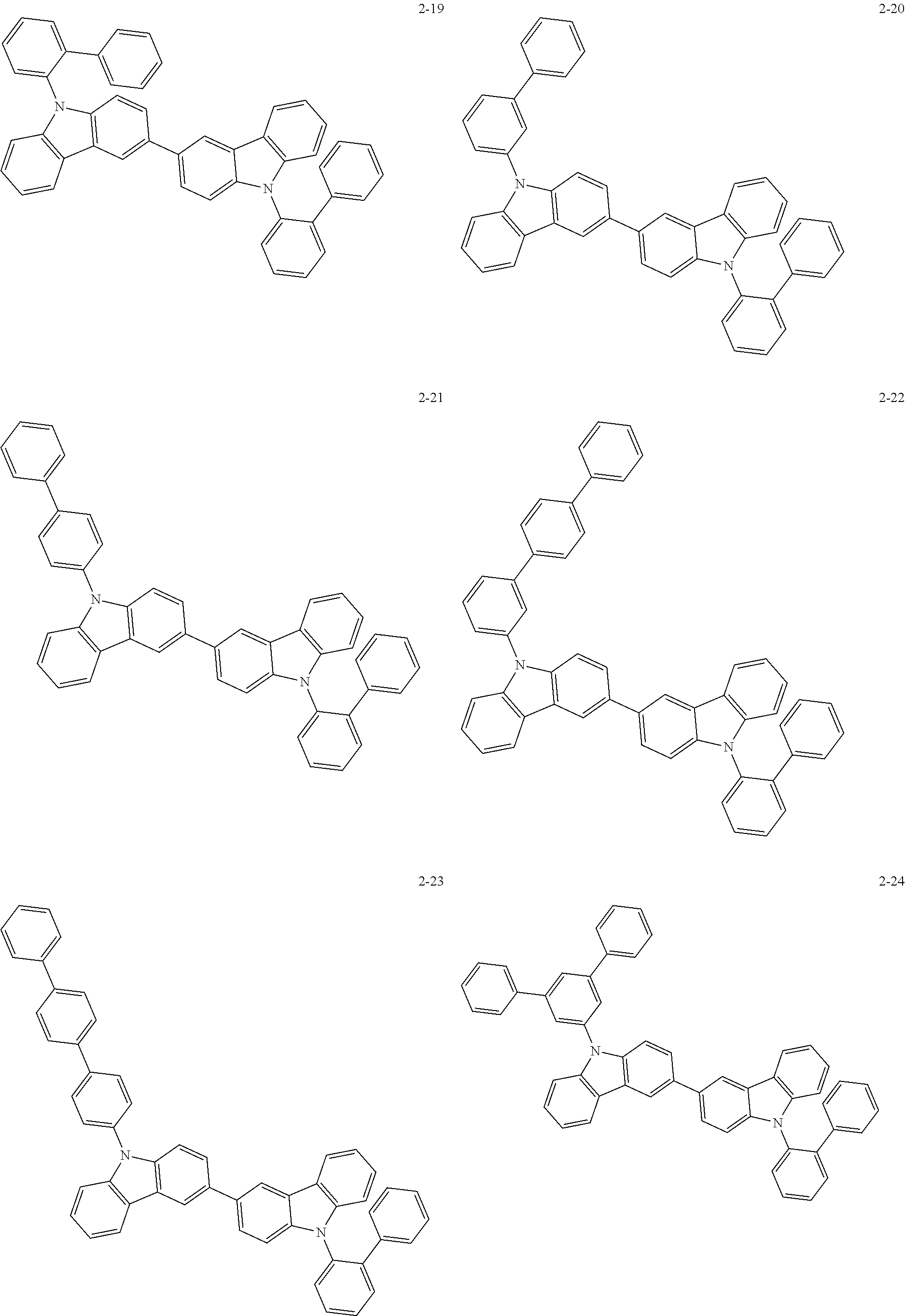

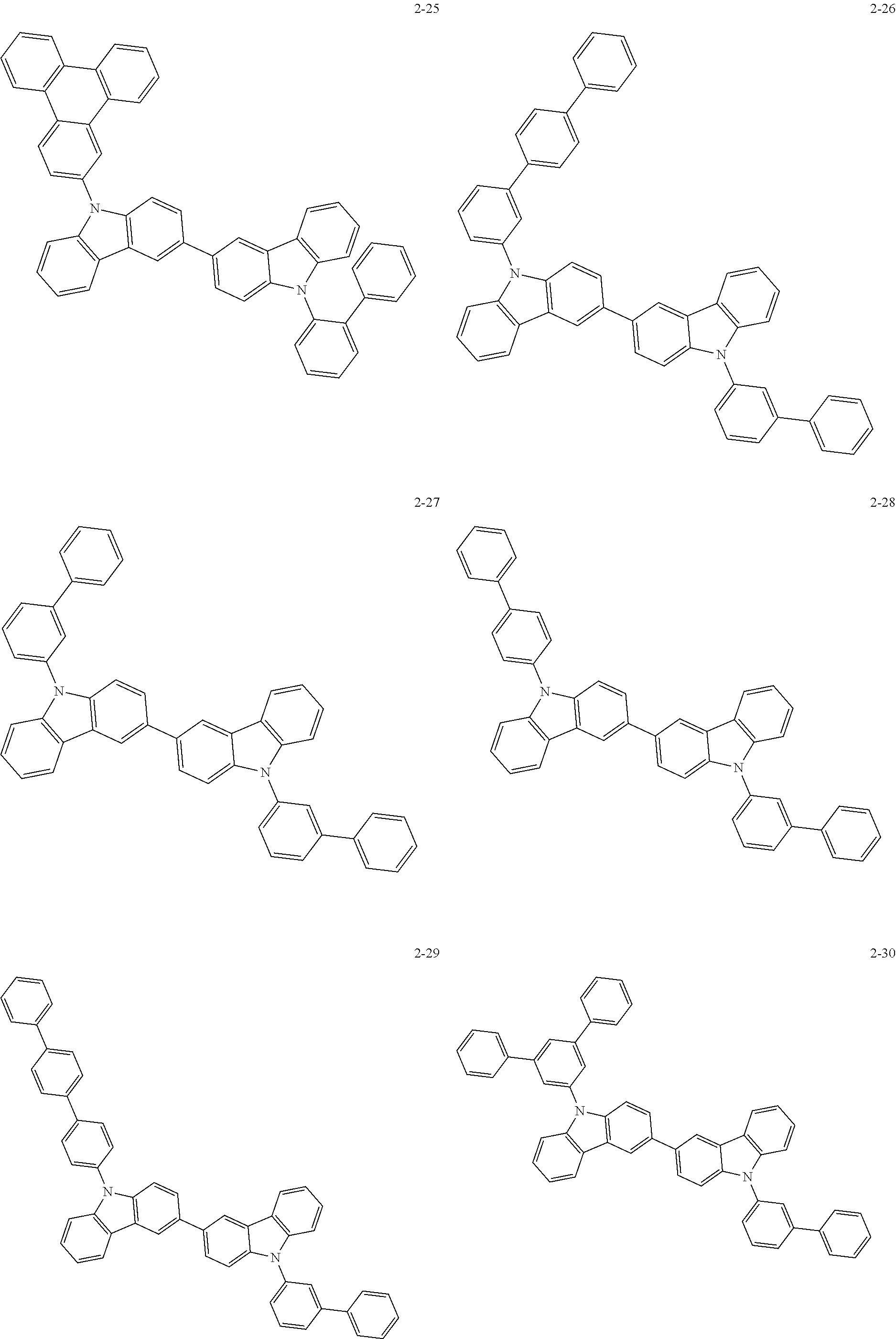

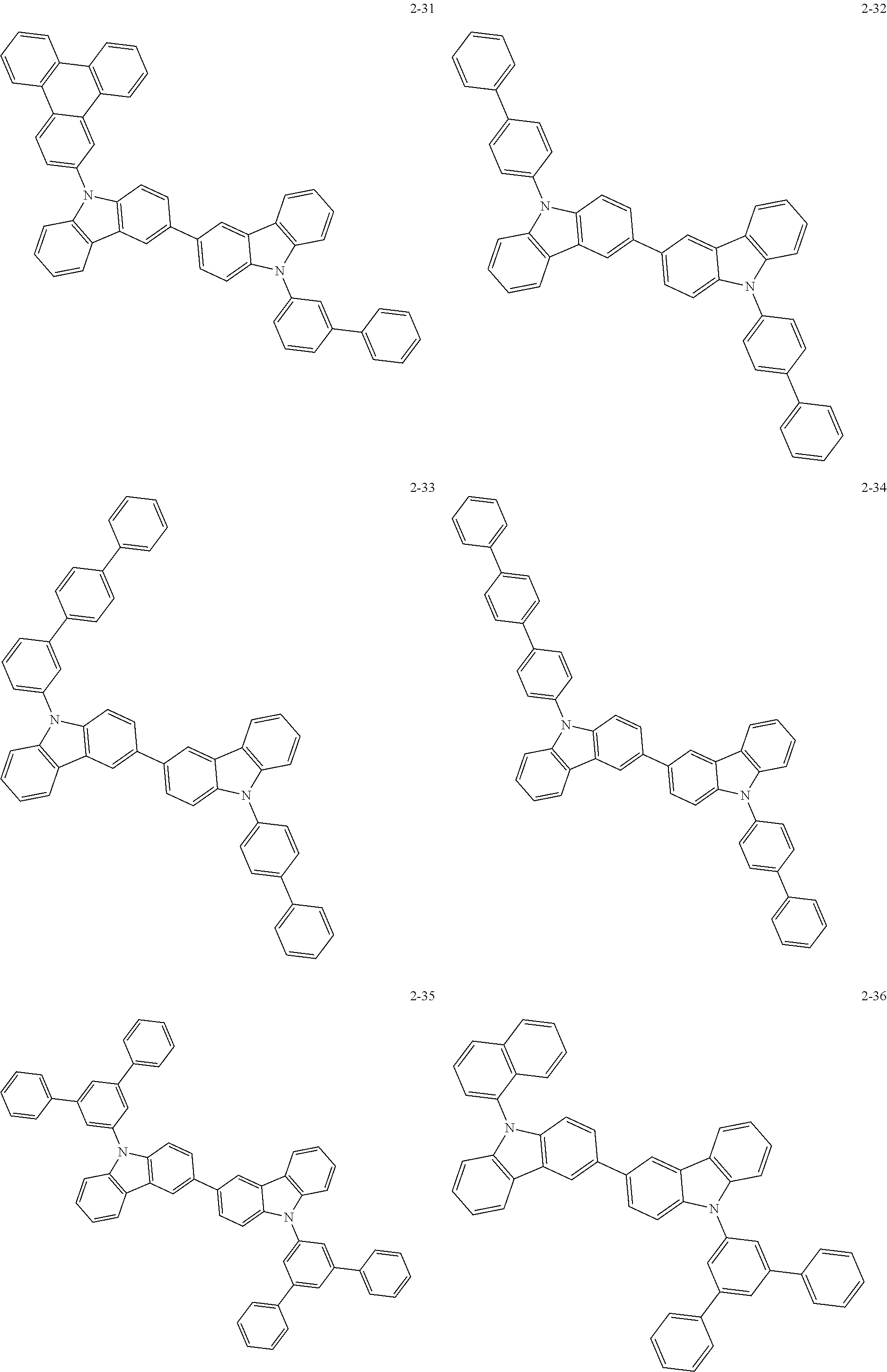

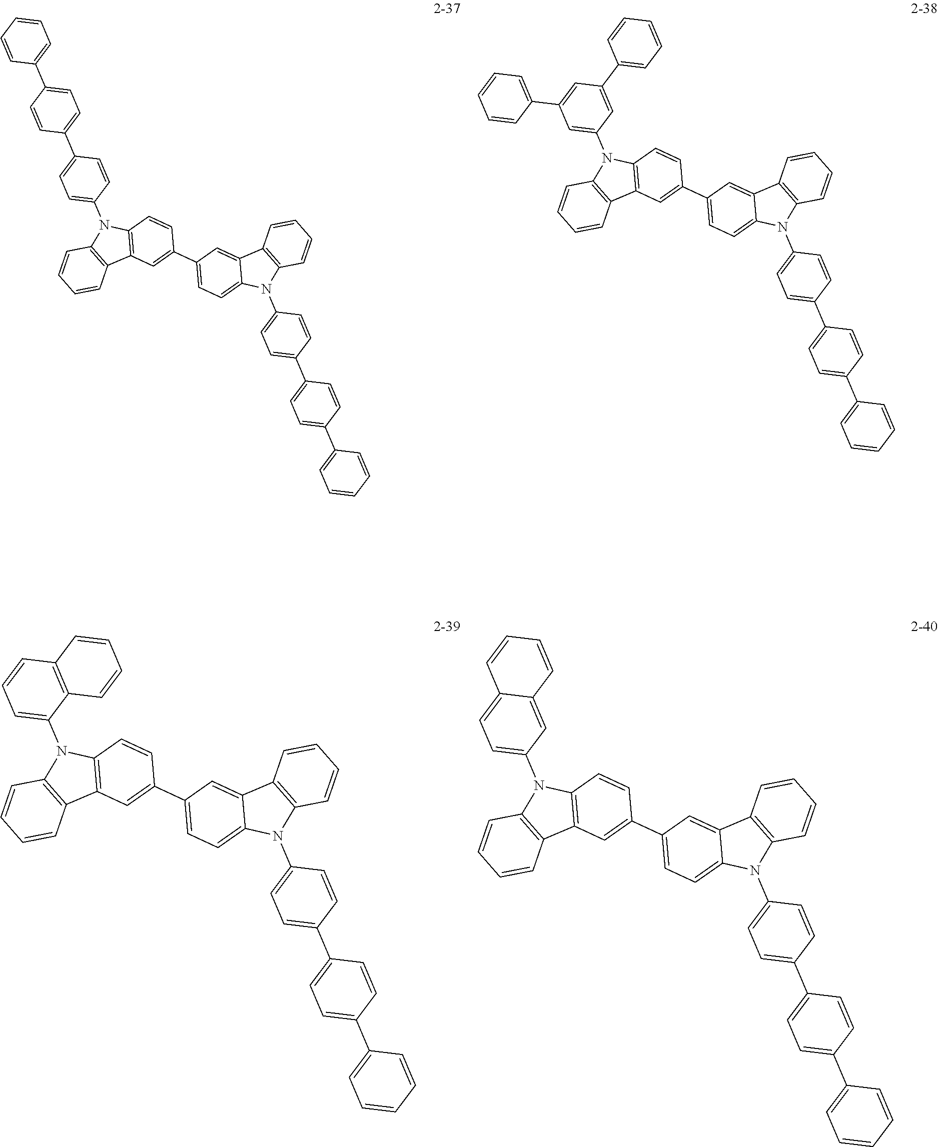 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

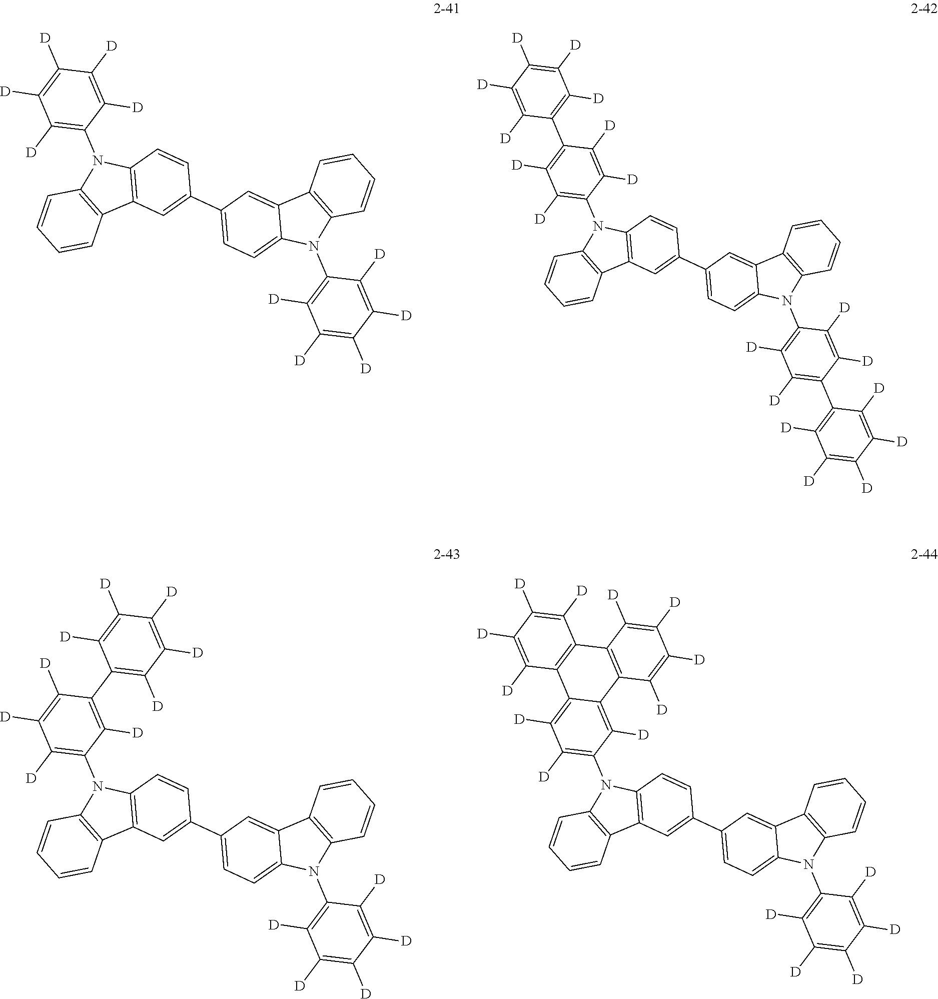

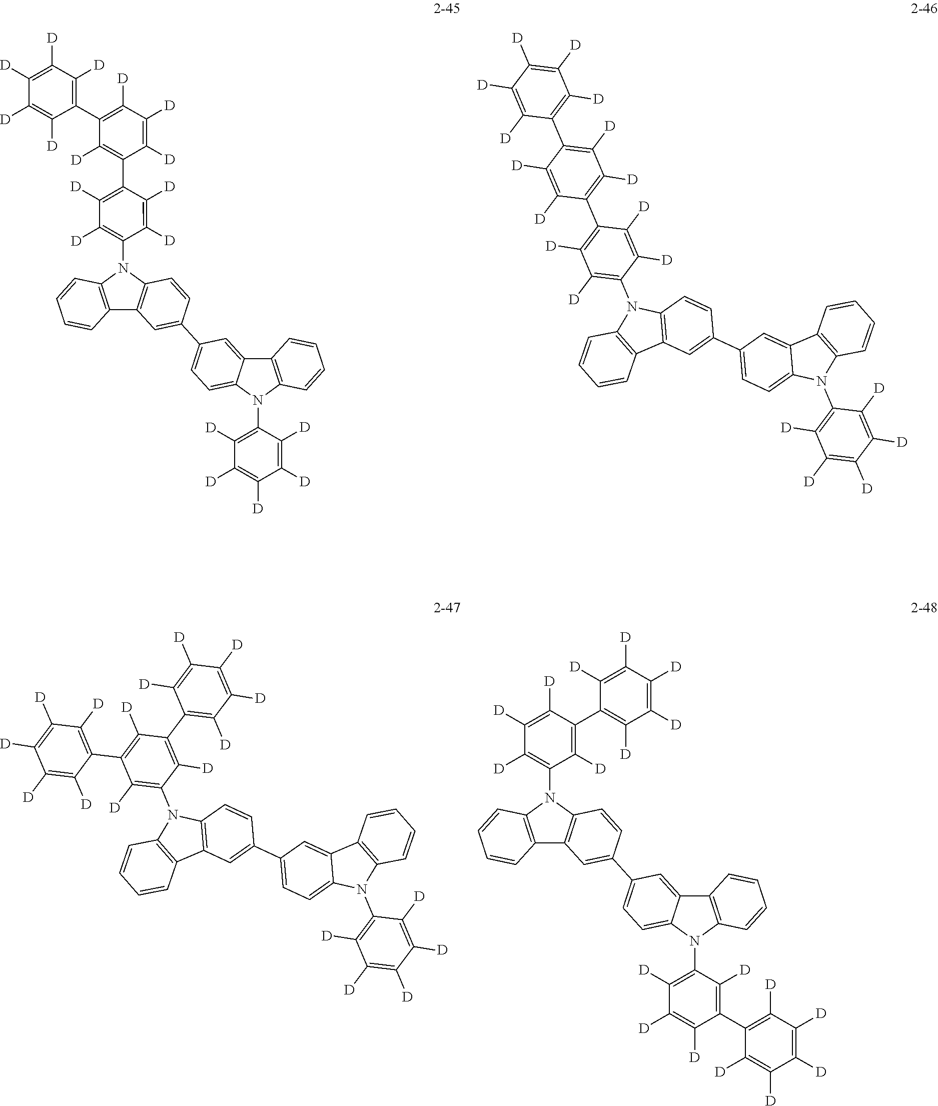

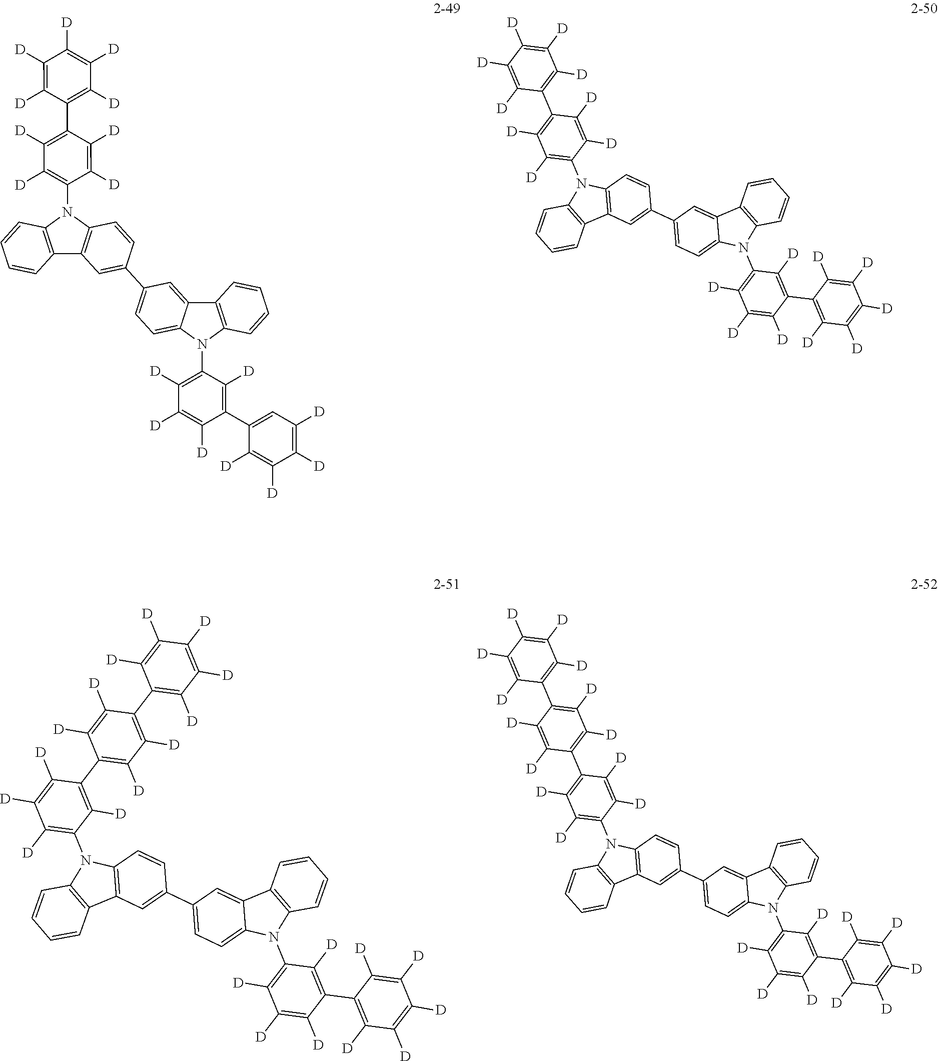

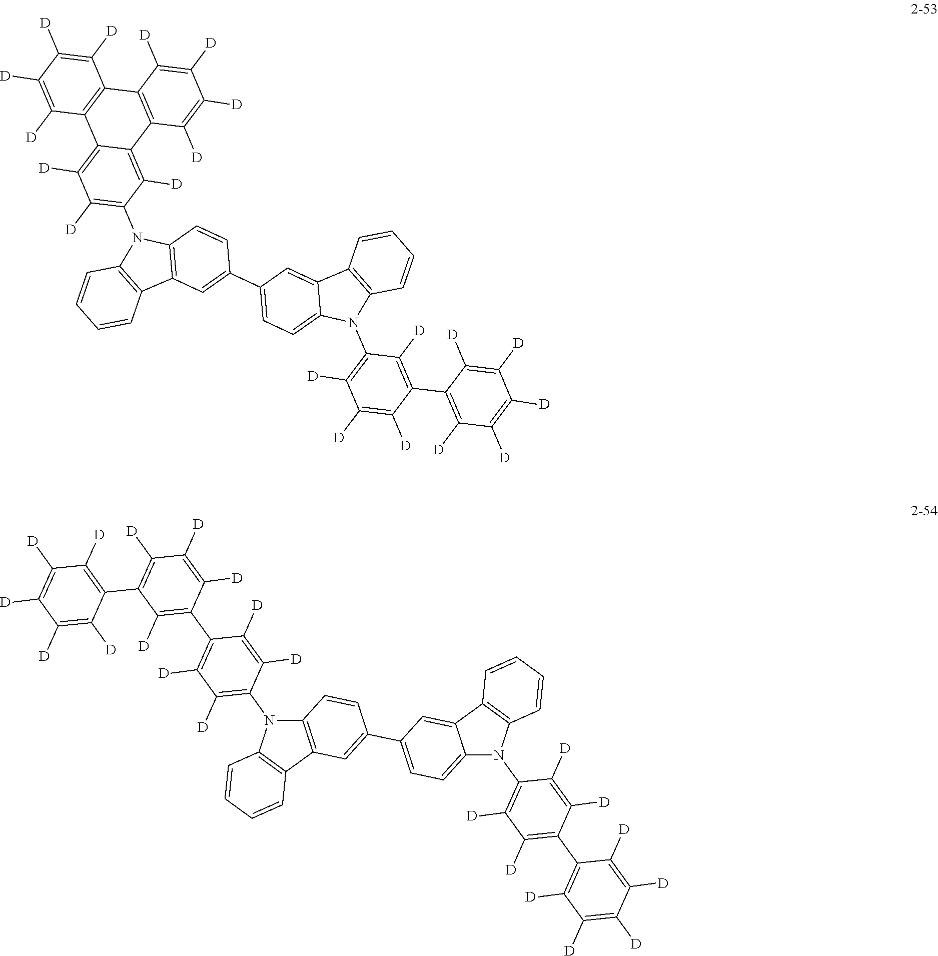

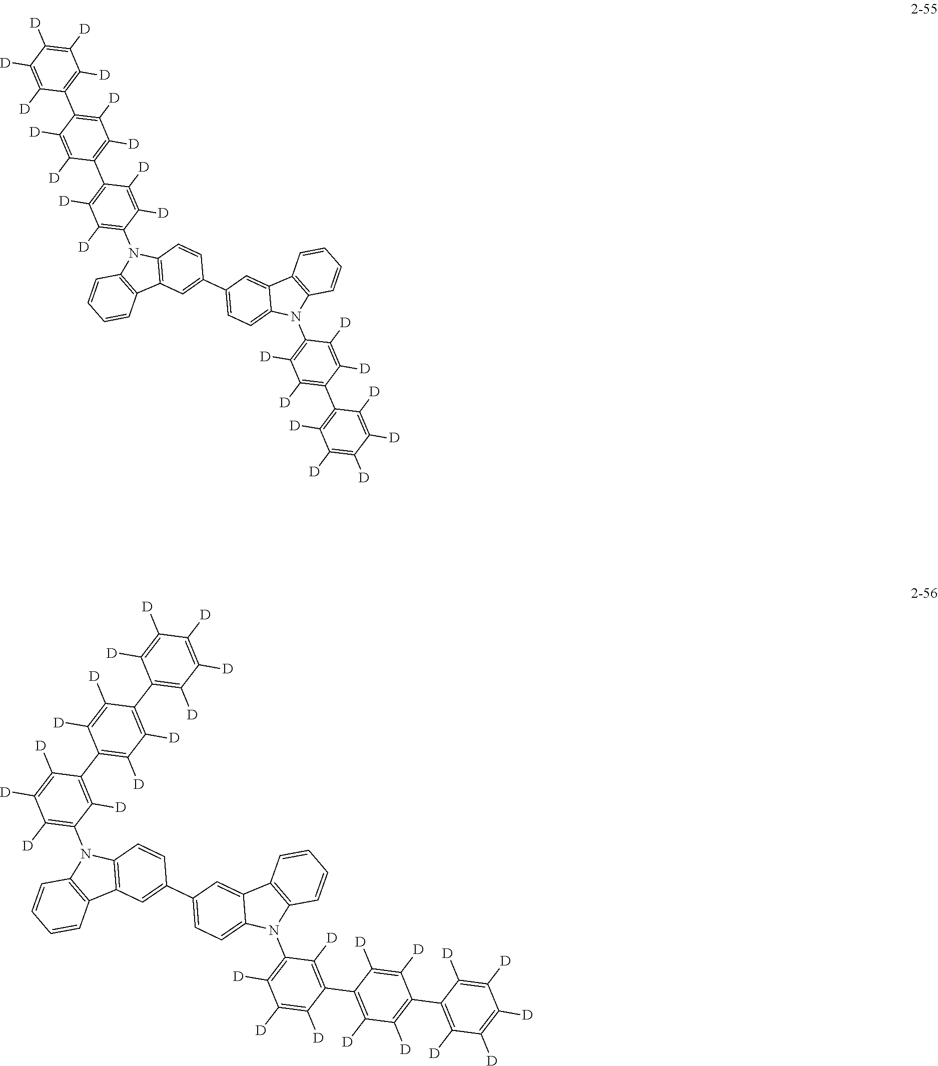

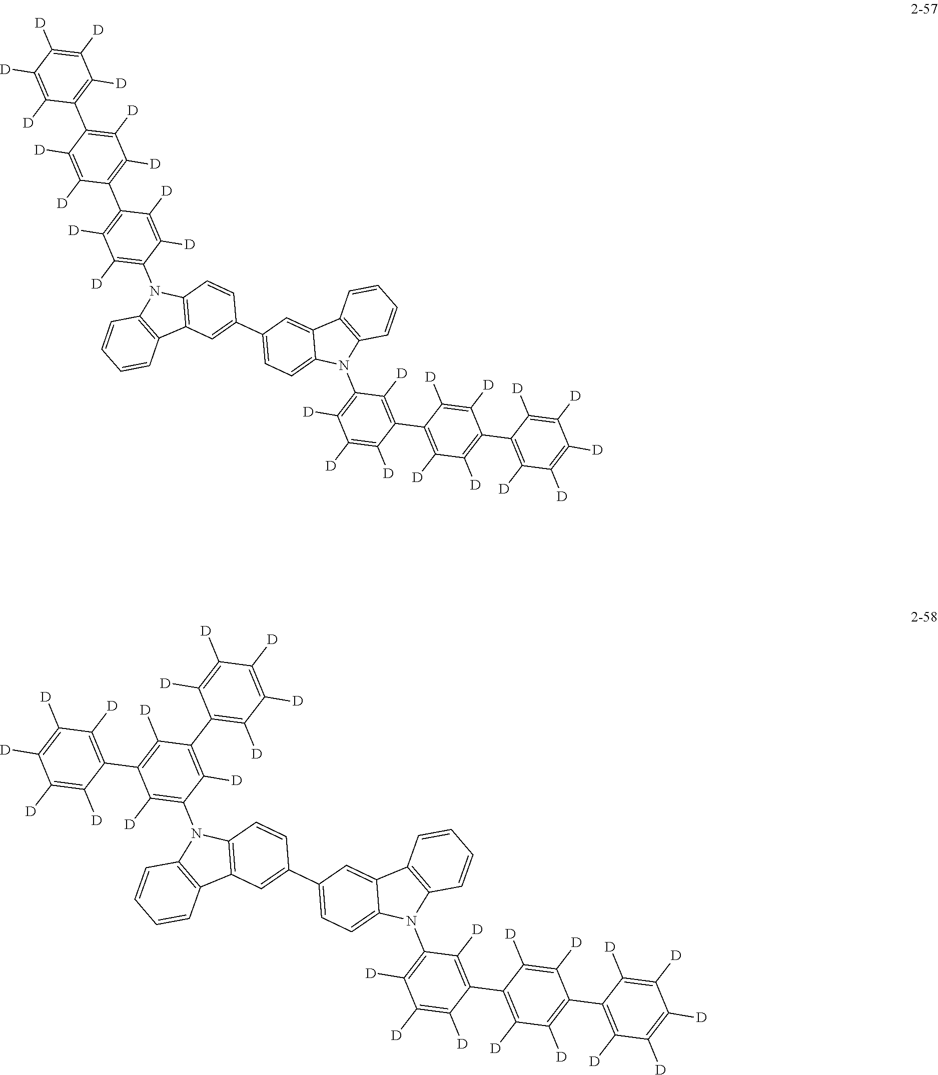

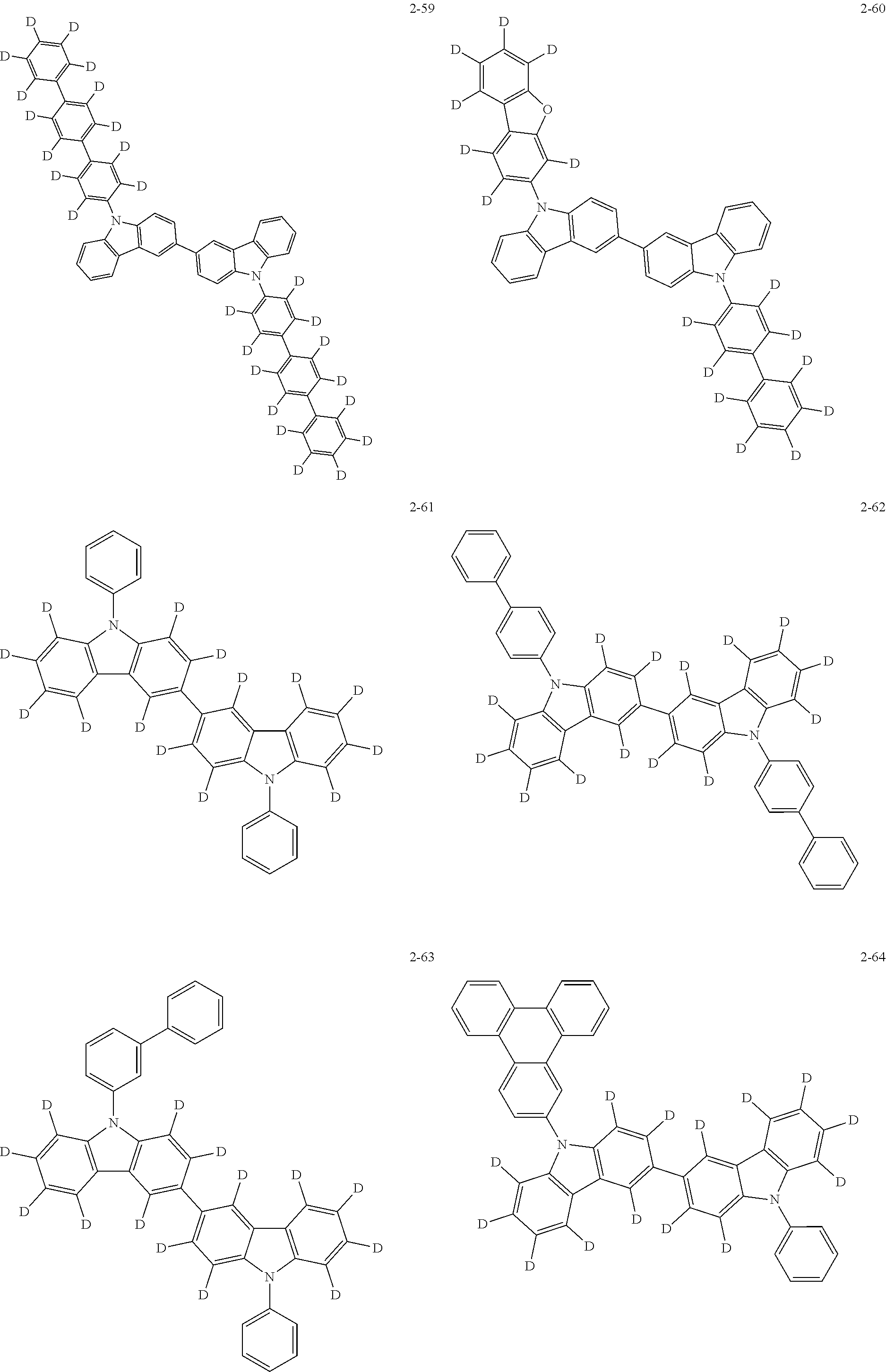

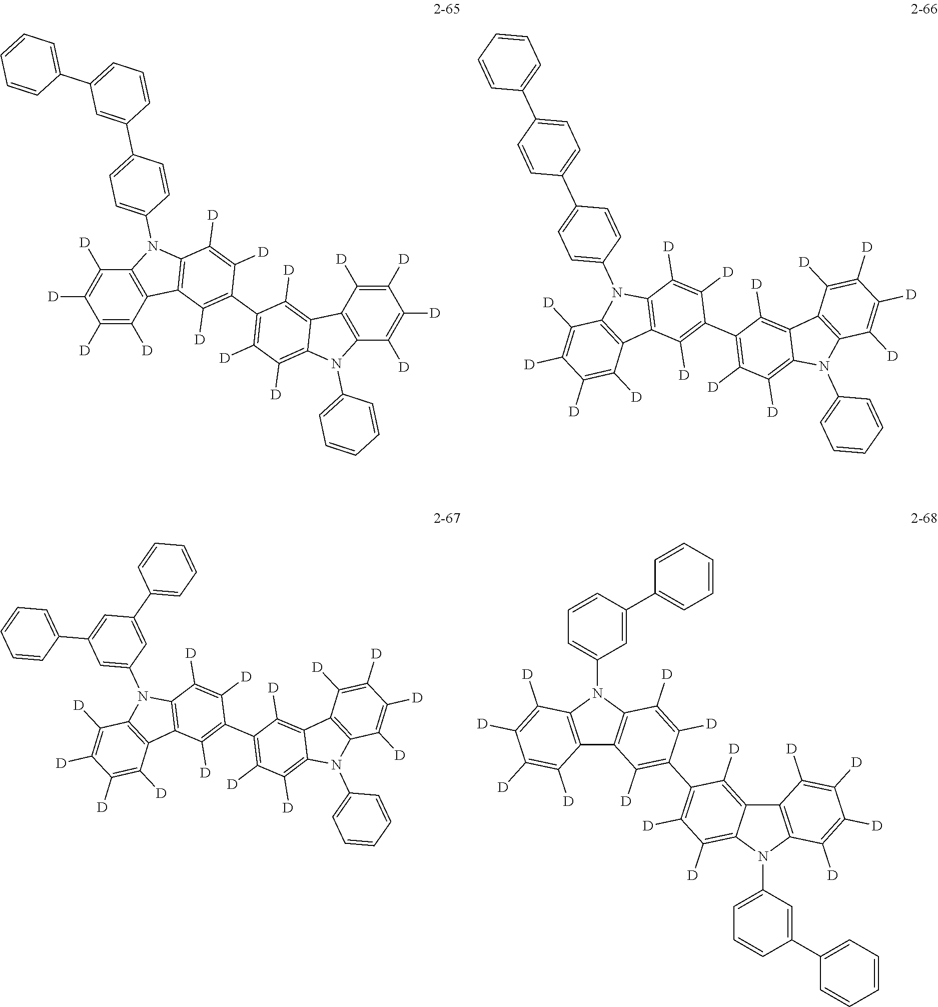

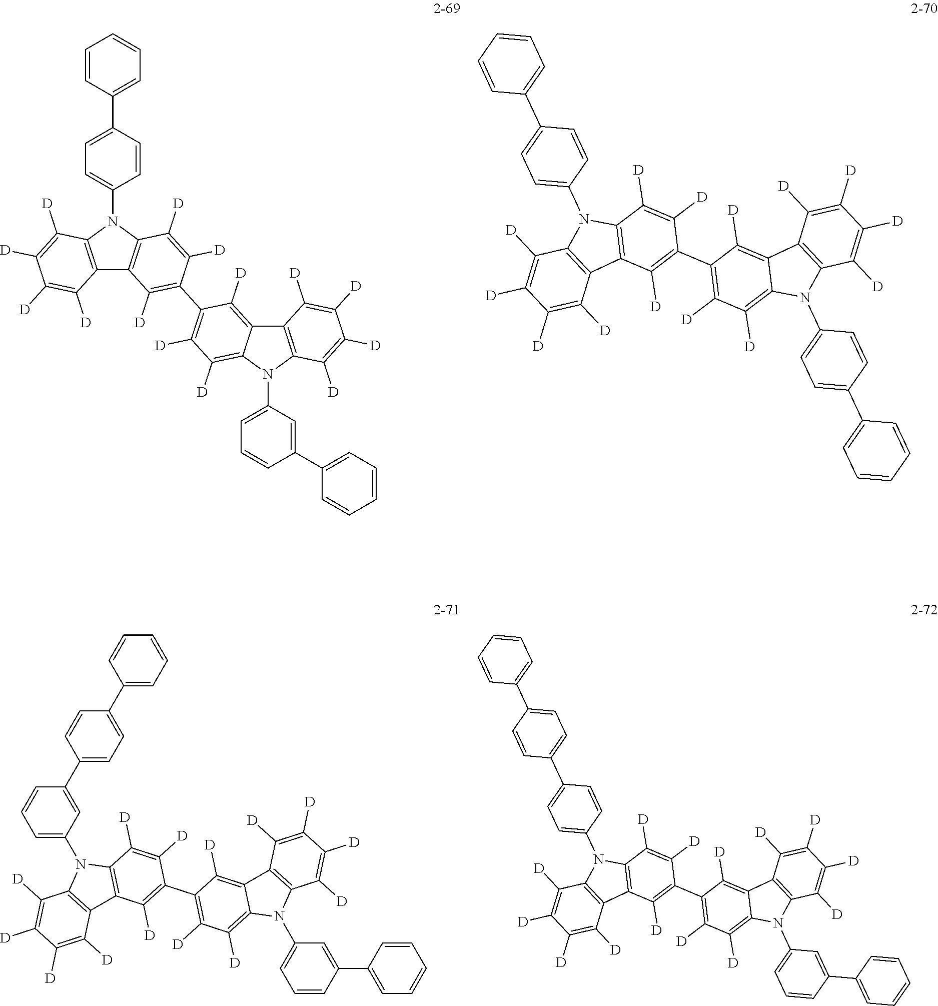

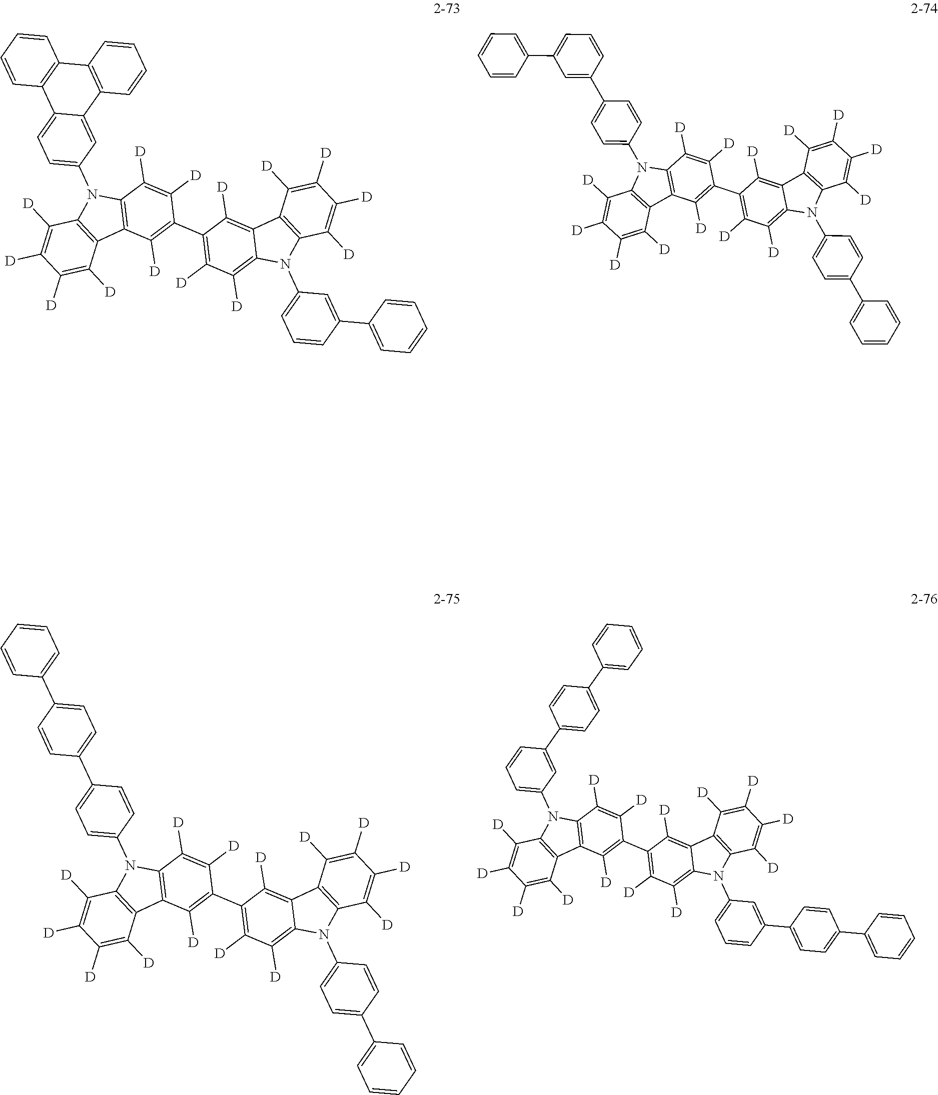

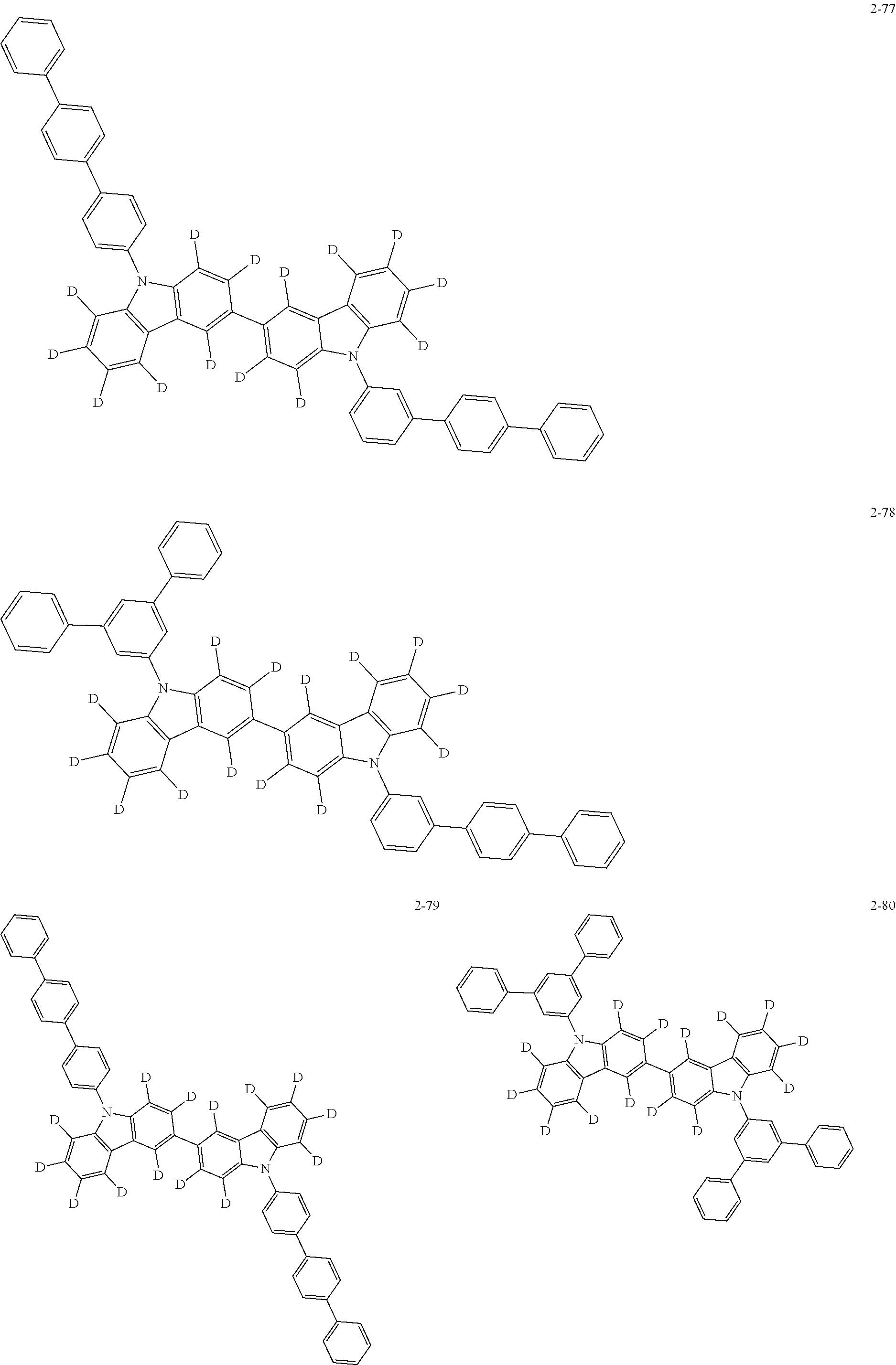

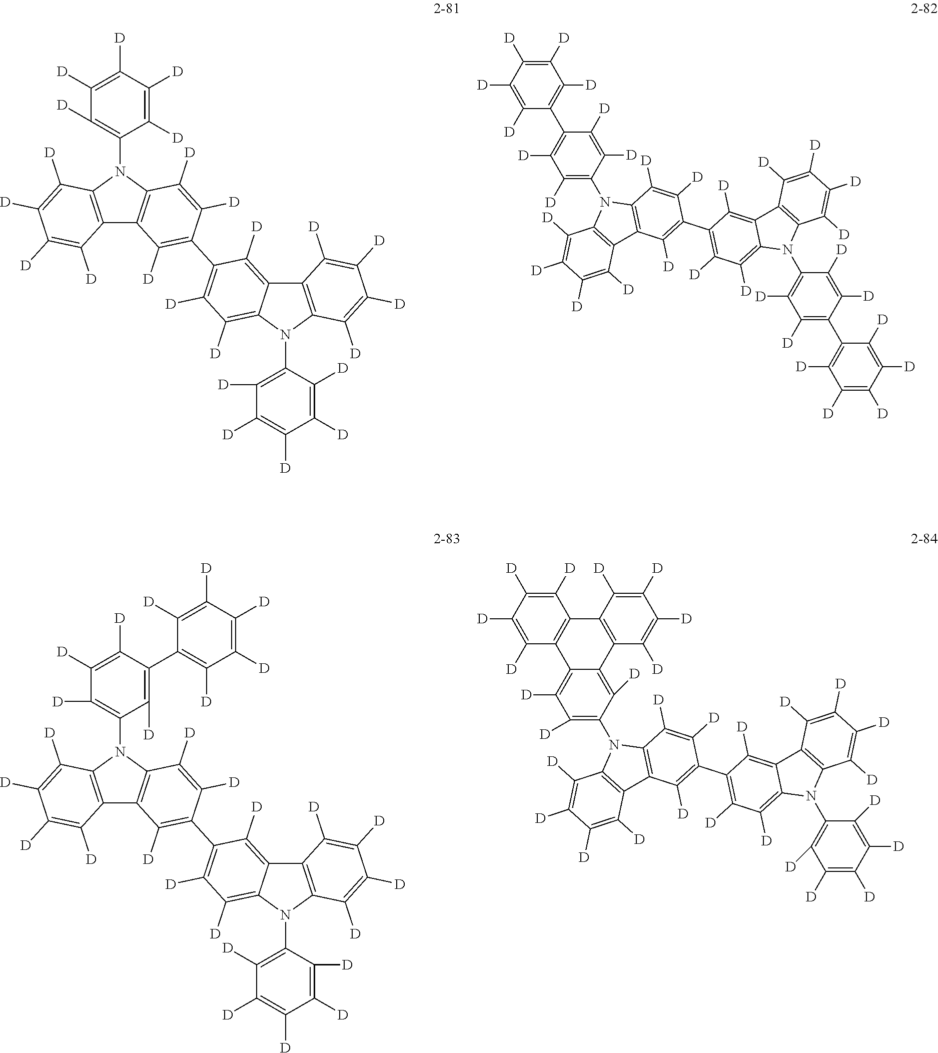

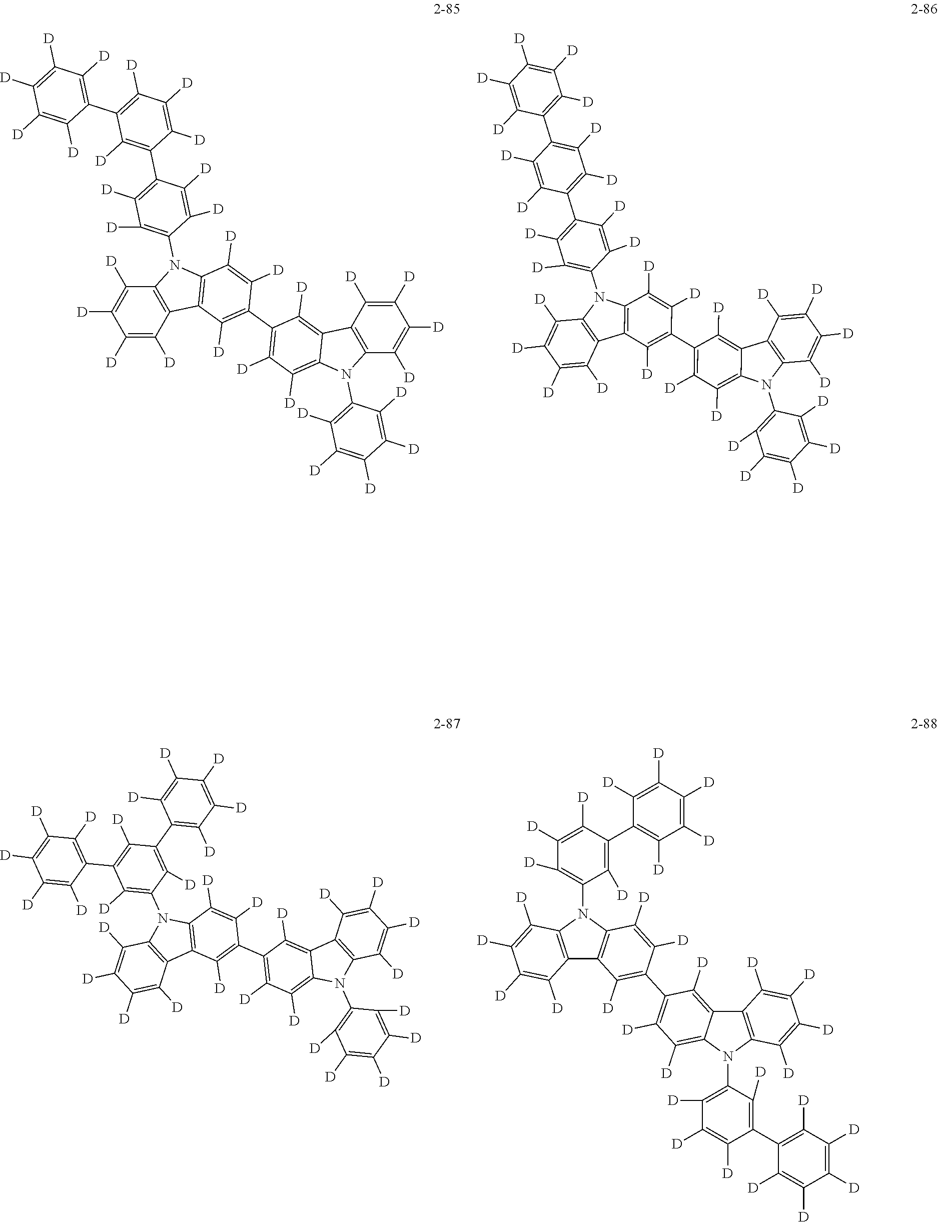

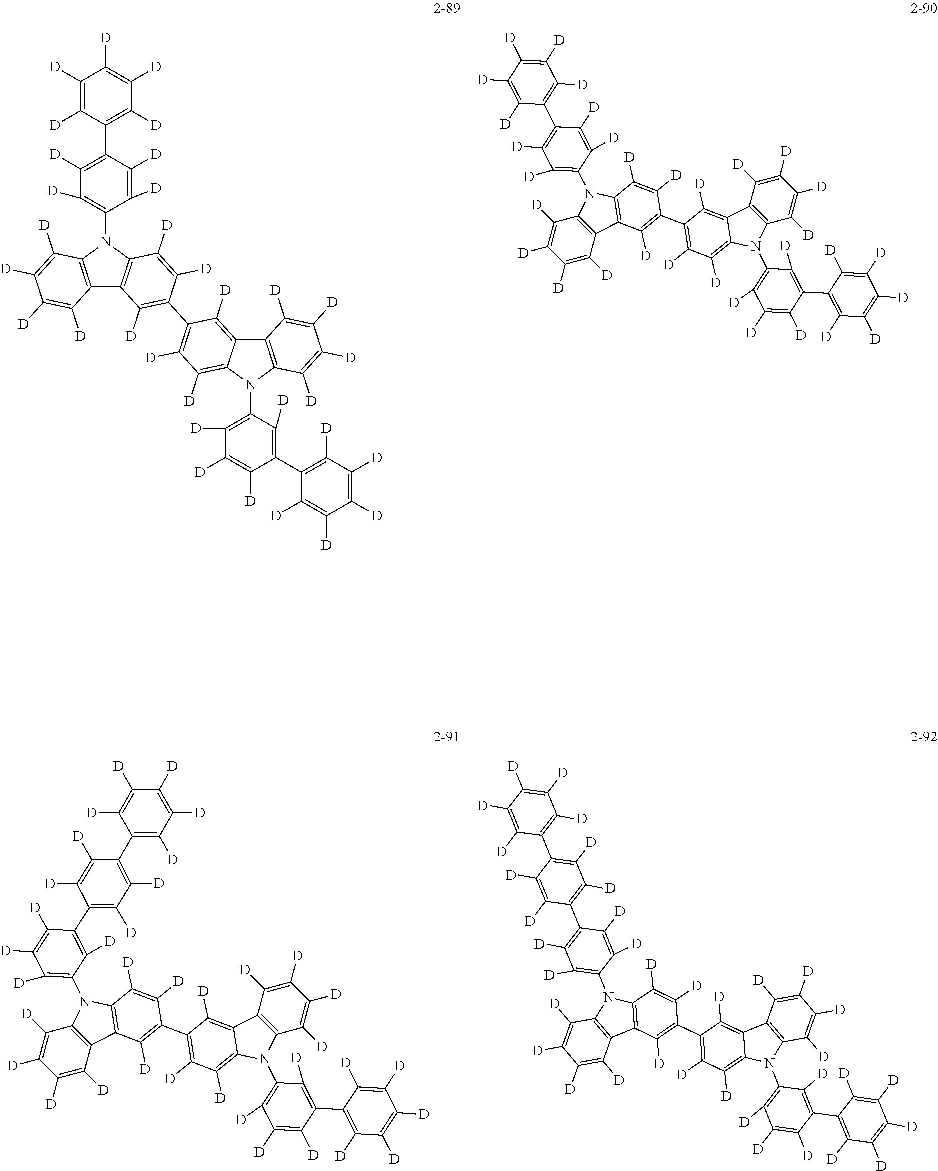

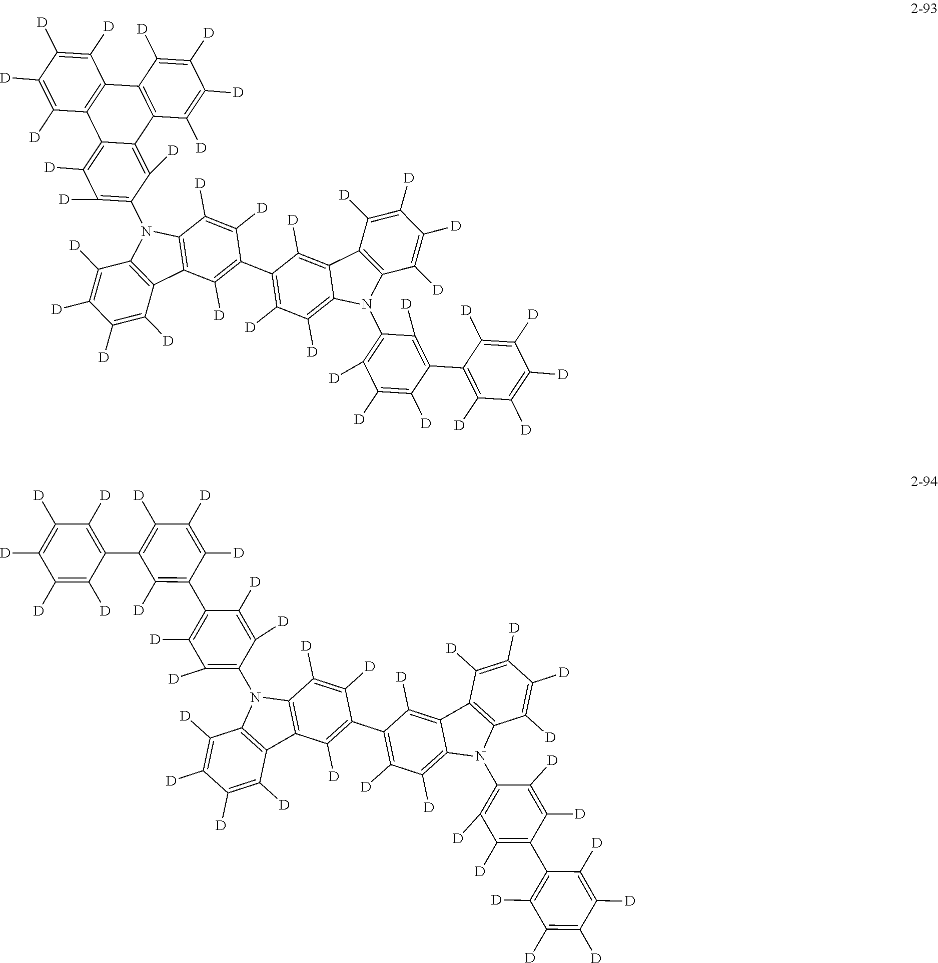

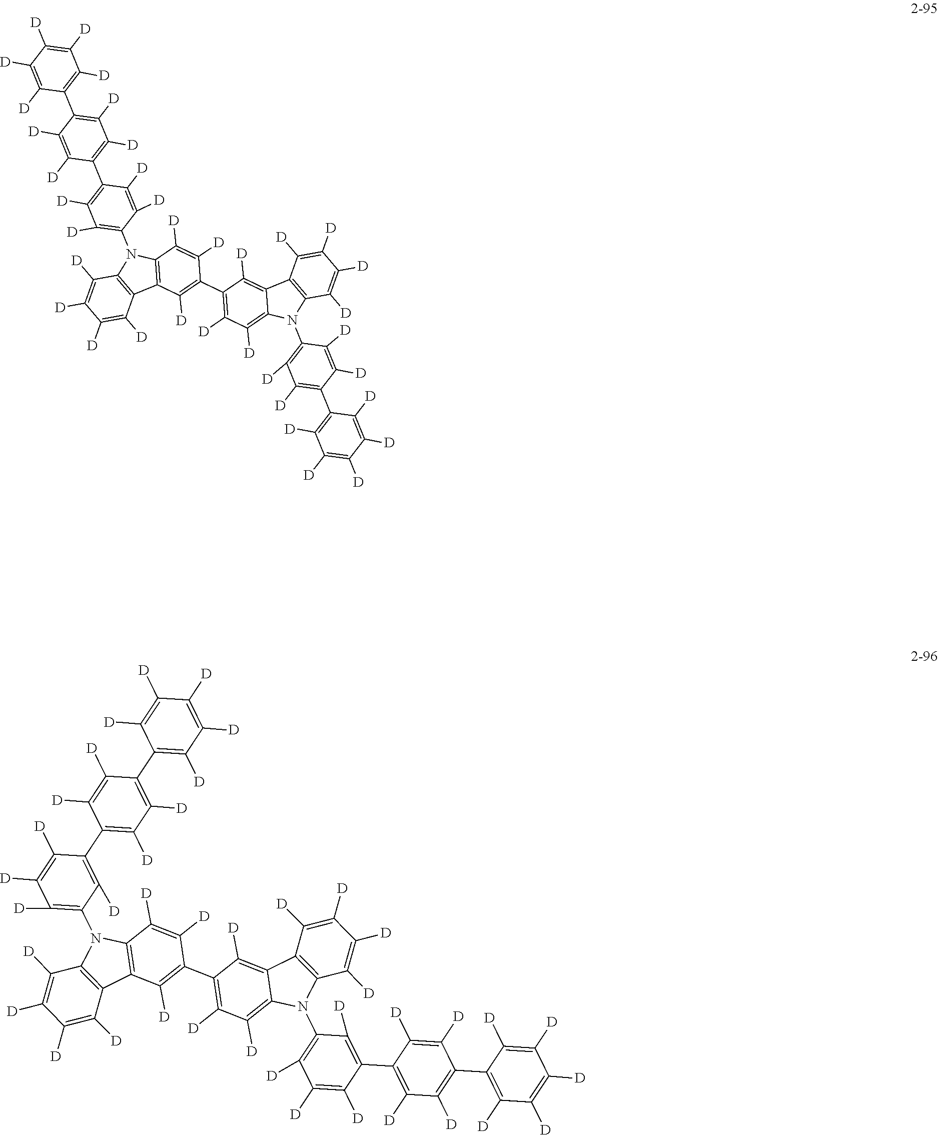

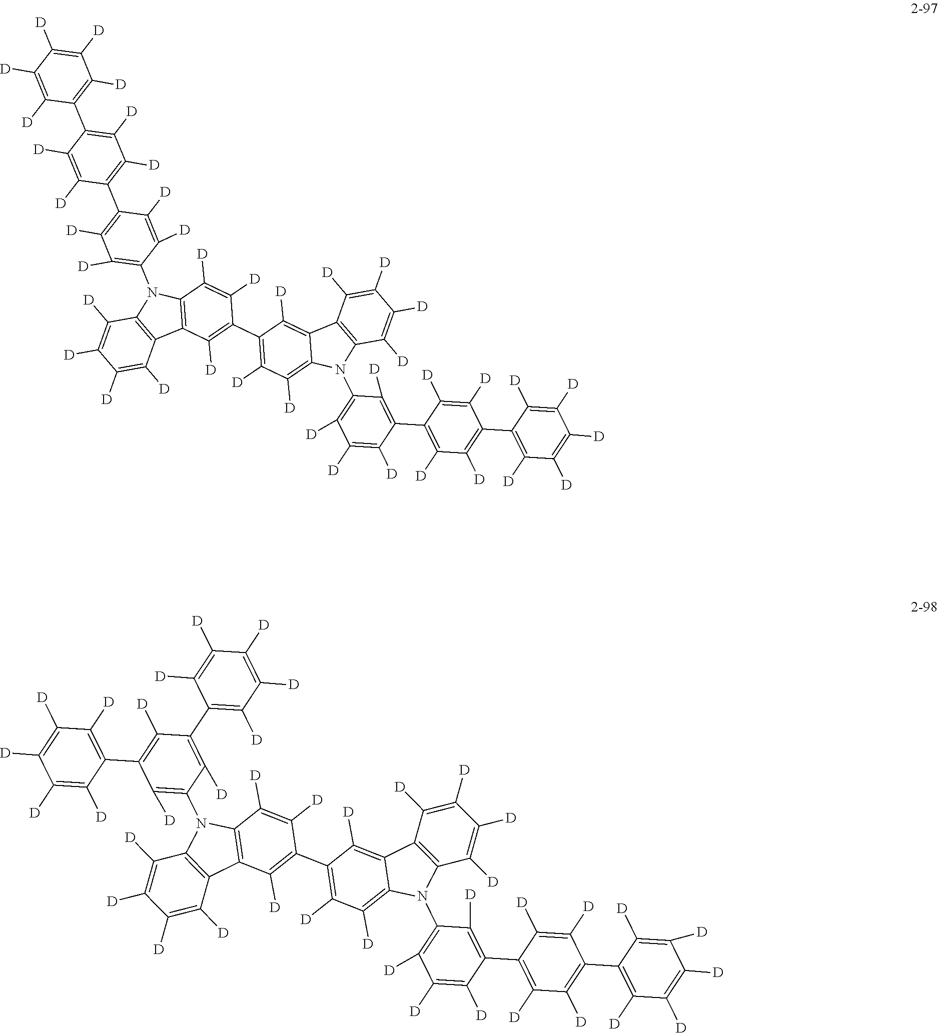

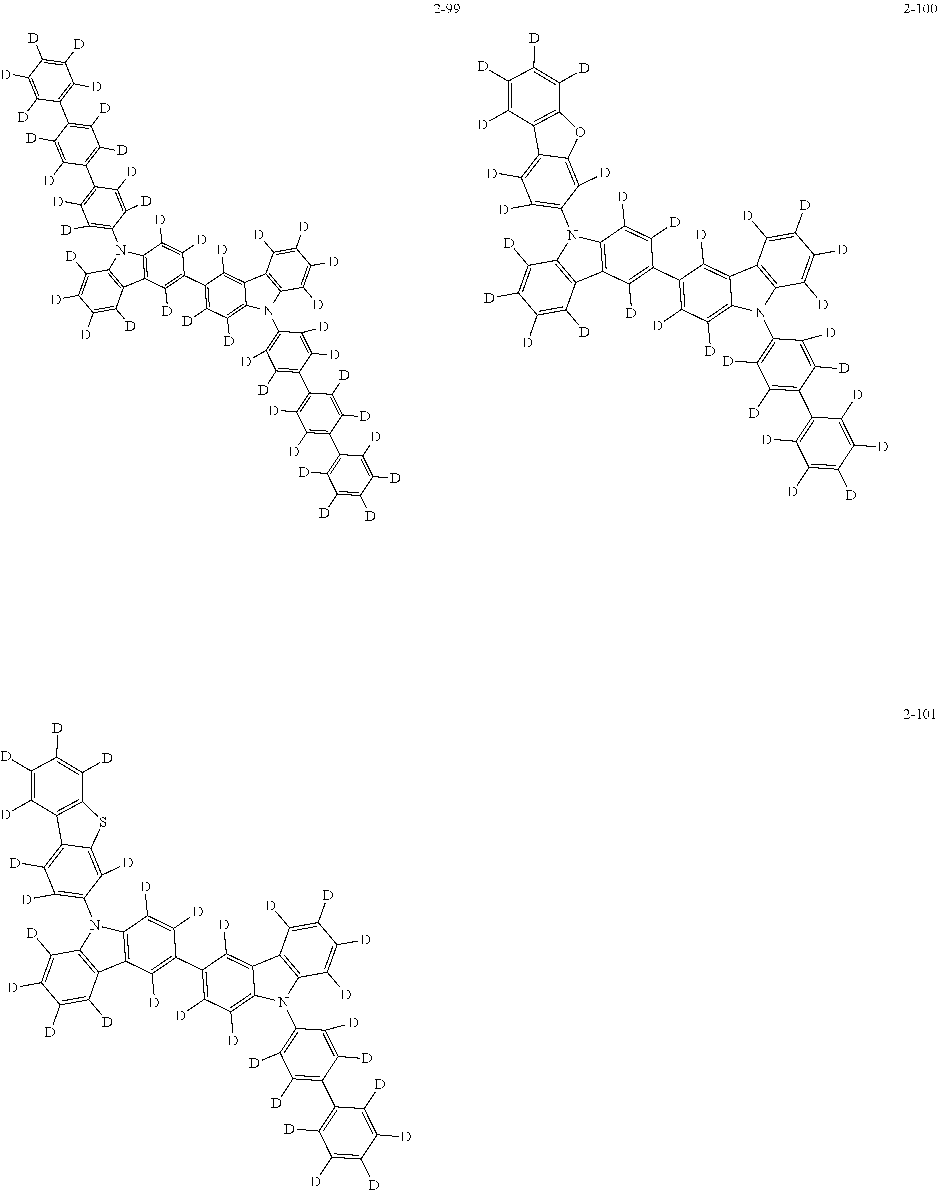

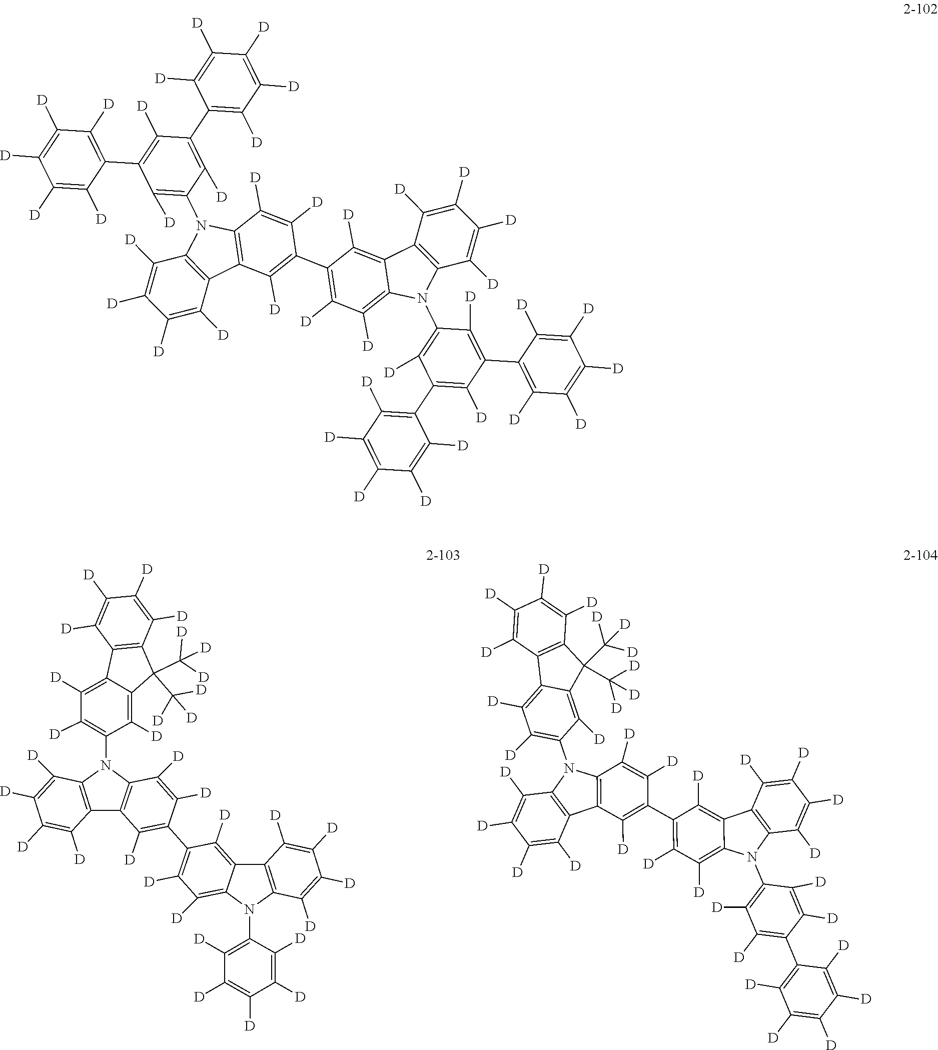

A content on the organic light emitting device including the heterocyclic compound represented by Chemical Formula 1 described above may be applied to the organic light emitting device further including the heterocyclic compound represented by Chemical Formula 2.

In another exemplary embodiment, the heterocyclic compound represented by Chemical Formula 2 can be used as a light emitting material for a light emitting layer of the organic light emitting device.

In still another exemplary embodiment, the heterocyclic compound represented by Chemical Formula 2 may be used as a light emitting material for a light emitting layer of an organic light emitting device, and may be used as a p-type host material.

In the organic light emitting device of the present invention, the organic material layer may include the heterocyclic compound represented by Chemical Formula 1 and the heterocyclic compound represented by Chemical Formula 2. The organic material layer may be formed by pre-mixing the heterocyclic compound represented by Chemical Formula 1 and the heterocyclic compound represented by Chemical Formula 2 and using a thermal vacuum deposition method.

In another organic light emitting device, the organic material layer includes a light emitting layer, the light emitting layer includes a host material, and the host material may include the heterocyclic compound represented by Chemical Formula 1 and the heterocyclic compound represented by Chemical Formula 2.

In still another organic light emitting device, the organic material layer includes a light emitting layer, includes the heterocyclic compound represented by Chemical Formula 1 as an n-type host material for the light emitting layer, and may include the heterocyclic compound represented by Chemical Formula 2 as a p-type host material.

In an exemplary embodiment of the present application, provided is a composition for an organic material layer of an organic light emitting device, which includes the heterocyclic compound represented by Chemical Formula 1 and the heterocyclic compound represented by Chemical Formula 2.

The weight ratio of the heterocyclic compound represented by Chemical Formula 1 the heterocyclic compound represented by Chemical Formula 2 in the composition may be 1:10 to 10:1, 1:8 to 8:1, 1:5 to 5:1, and 1:2 to 2:1, but is not limited thereto. The composition may be used when an organic material layer of an organic light emitting device is formed, and particularly, may be more preferably used as a host material for the light emitting layer.

The composition is in a form in which two or more compounds are simply mixed, materials in a powder state may also be mixed before an organic material layer of an organic light emitting device is formed, and it is possible to mix compounds in a liquid state at a temperature which is equal to or more than a suitable temperature. The composition is in a solid state at a temperature which is equal to or less than the melting point of each material, and may be maintained as a liquid phase when the temperature is adjusted.

The composition may additionally include materials publicly known in the art such as solvents and additives.

Hereinafter, the present specification will be described in more detail through Examples, but these Examples are provided only for exemplifying the present application, and are not intended to limit the scope of the present application.

### <Preparation Example 1> Preparation of Compound 1-1(C)

### Preparation of Intermediate 1 (A)

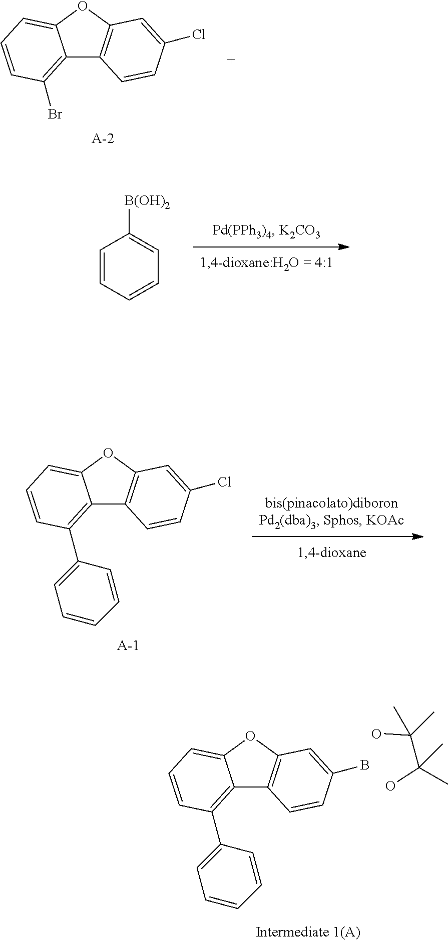

### Synthesis Method of Intermediate A-1

A mixture of 1-bromo-7-chlorodibenzo[b,d]furan (15 g, 53.28 mmol), phenylboronic acid (7.79 g, 63.93 mmol), tetrakis(triphenylphosphine)palladium(0) (3.07 g, 2.66 mmol), potassium carbonate (22.09 g, 159.84 mmol), and 1,4-dioxane/distilled water (150 ml/37.5 ml) was refluxed at 120° C. in a reaction flask for 3 hours. After the reaction was completed, the temperature was lowered to room temperature, and then the resulting solid was washed with distilled water and methanol (MeOH) to obtain Intermediate A-1. (14 g, 94%)

### Synthesis Method of Intermediate 1(A)

After Intermediate A-1 (14 g, 62 mmol), bis(pinacolato)diboron (25.5 g, 100 mmol), Sphos (4.1 g, 10 mmol), KOAc (14.7 g, 150 mmol), and Pd₂(dba)₃ (4.5 g, 5 mmol) were put into a reaction flask, 140 ml of 1,4-dioxane was added thereto, and then the resulting mixture was heated at 120° C. for 4 hours. After the reaction was completed, the base was removed, and then the solvent was concentrated and then subjected to column purification to obtain Intermediate 1(A). (16 g, 86%)

### Synthesis Method of Compound 1-1(C)

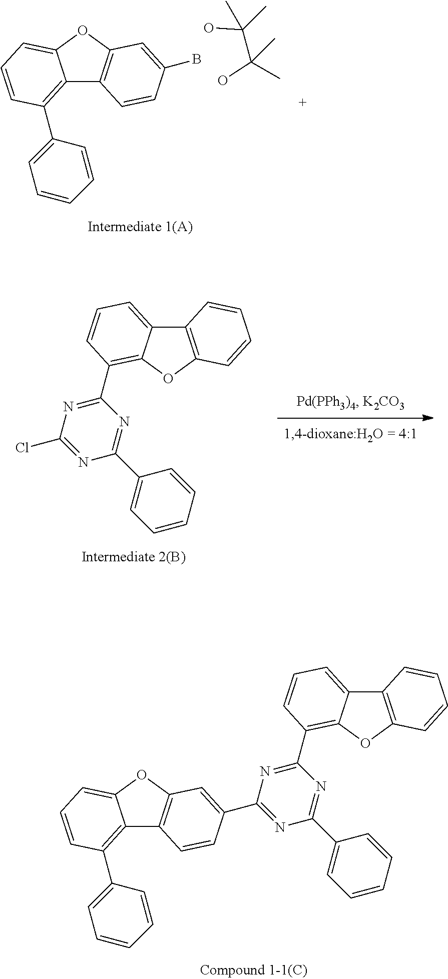

A mixture of Intermediate 1(A) (15 g, 40.5 mmol), Intermediate 2(B) (17.3 g, 48.6 mmol), tetrakis(triphenylphosphine)palladium(0) (2.33 g, 2.02 mmol), potassium carbonate (16.8 g, 121.5 mmol), and 1,4-dioxane/water (150 ml/37.5 ml) was refluxed at 120° C. in a reaction flask for 3 hours. After the reaction was completed, the temperature was lowered to room temperature, and then the resulting solid was washed with distilled water and methanol (MeOH) to obtain Compound 1-1(C). (18 g, 81%)

The following Compound (C) was synthesized in the same manner as in Preparation Example 1 using compounds in the following Table 1 instead of Intermediate 1 (A) and Intermediate 2 (B).

TABLE 1

| Compound | Intermediate | Intermediate | | Yield |
|---|---|---|---|---|
| No. | 1 (A) | 2 (B) | Compound (C) | (%) |
| 1-2 | 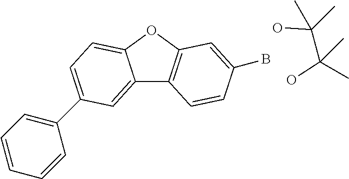 | 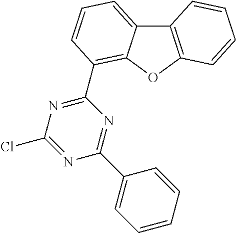 [SMILES: Clc1nc(-c2ccccc2)nc(-c2cccc3c2oc2ccccc23)n1] | 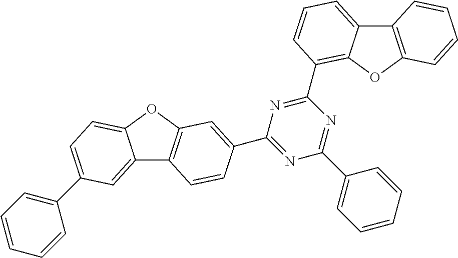 [SMILES: c1ccc(-c2ccc3oc4cc(-c5nc(-c6ccccc6)nc(-c6cccc7c6oc6ccccc67)n5)ccc4c3c2)cc1] | 85 |
| 1-3 | 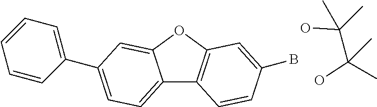 | 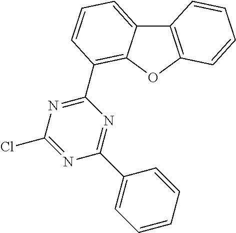 [SMILES: Clc1nc(-c2ccccc2)nc(-c2cccc3c2oc2ccccc23)n1] | 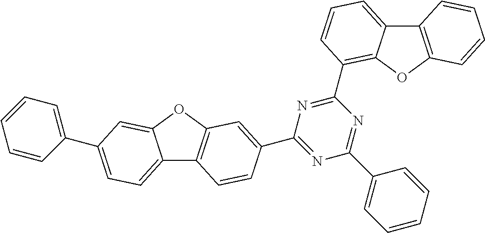 [SMILES: c1ccc(-c2ccc3c(c2)oc2cc(-c4nc(-c5ccccc5)nc(-c5cccc6c5oc5ccccc56)n4)ccc23)cc1] | 83 |
| 1-4 | 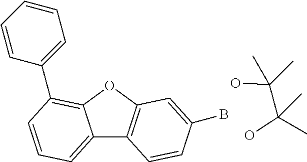 | 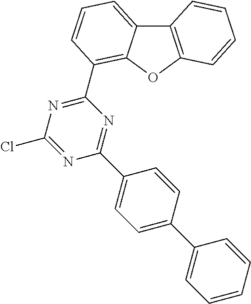 [SMILES: Clc1nc(-c2ccc(-c3ccccc3)cc2)nc(-c2cccc3c2oc2ccccc23)n1] | 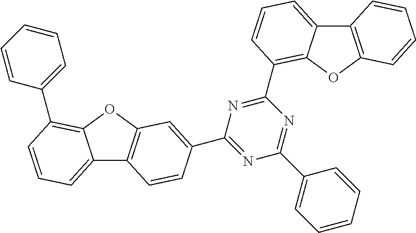 [SMILES: c1ccc(-c2nc(-c3ccc4c(c3)oc3c(-c5ccccc5)cccc34)nc(-c3cccc4c3oc3ccccc34)n2)cc1] | 80 |
| 1-5 | 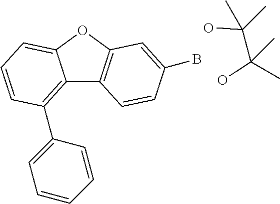 | 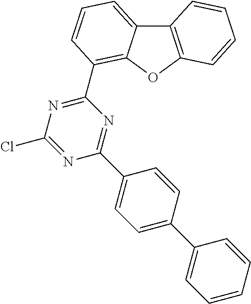 [SMILES: Clc1nc(-c2ccc(-c3ccccc3)cc2)nc(-c2cccc3c2oc2ccccc23)n1] | 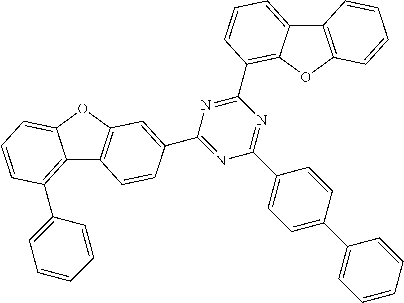 [SMILES: c1ccc(-c2ccc(-c3nc(-c4ccc5c(c4)oc4cccc(-c6ccccc6)c45)nc(-c4cccc5c4oc4ccccc45)n3)cc2)cc1] | 81 |
| 1-6 | 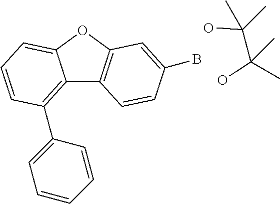 | 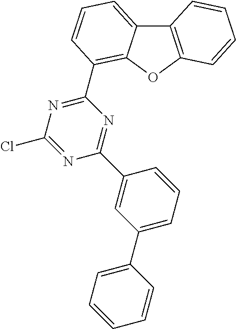 [SMILES: Clc1nc(-c2cccc(-c3ccccc3)c2)nc(-c2cccc3c2oc2ccccc23)n1] | 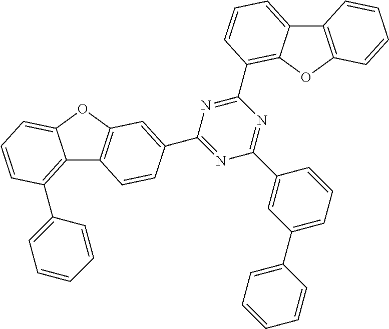 [SMILES: c1ccc(-c2cccc(-c3nc(-c4ccc5c(c4)oc4cccc(-c6ccccc6)c45)nc(-c4cccc5c4oc4ccccc45)n3)c2)cc1] | 82 |
| 1-7 | 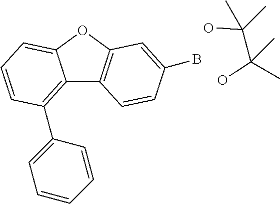 | 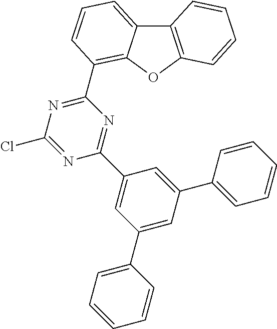 [SMILES: Clc1nc(-c2cc(-c3ccccc3)cc(-c3ccccc3)c2)nc(-c2cccc3c2oc2ccccc23)n1] | 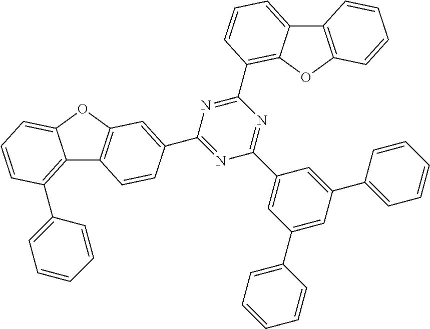 [SMILES: c1ccc(-c2cc(-c3ccccc3)cc(-c3nc(-c4ccc5c(c4)oc4cccc(-c6ccccc6)c45)nc(-c4cccc5c4oc4ccccc45)n3)c2)cc1] | 83 |
| 1-8 | 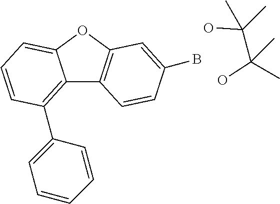 | 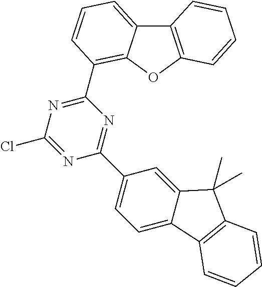 [SMILES: CC1(C)c2ccccc2-c2ccc(-c3nc(Cl)nc(-c4cccc5c4oc4ccccc45)n3)cc21] | 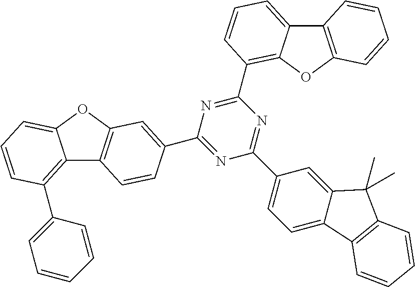 [SMILES: CC1(C)c2ccccc2-c2ccc(-c3nc(-c4ccc5c(c4)oc4cccc(-c6ccccc6)c45)nc(-c4cccc5c4oc4ccccc45)n3)cc21] | 88 |
| 1-9 | 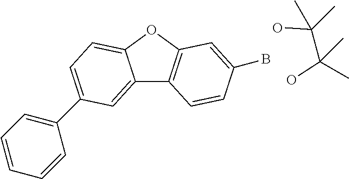 | 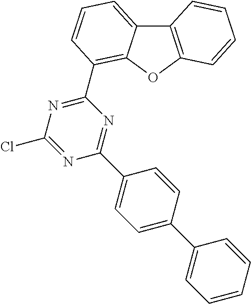 [SMILES: Clc1nc(-c2ccc(-c3ccccc3)cc2)nc(-c2cccc3c2oc2ccccc23)n1] | 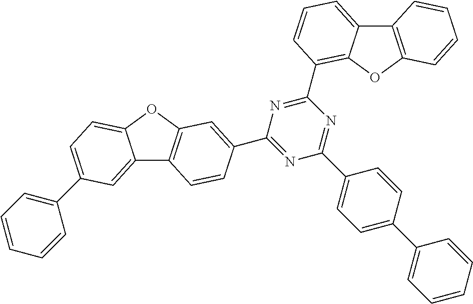 [SMILES: c1ccc(-c2ccc(-c3nc(-c4ccc5c(c4)oc4ccc(-c6ccccc6)cc45)nc(-c4cccc5c4oc4ccccc45)n3)cc2)cc1] | 85 |
| 1-10 | 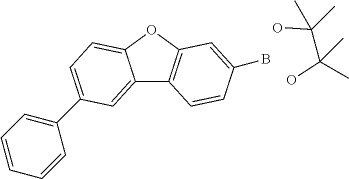 | 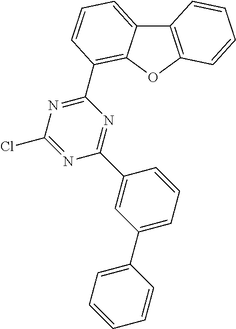 [SMILES: Clc1nc(-c2cccc(-c3ccccc3)c2)nc(-c2cccc3c2oc2ccccc23)n1] | 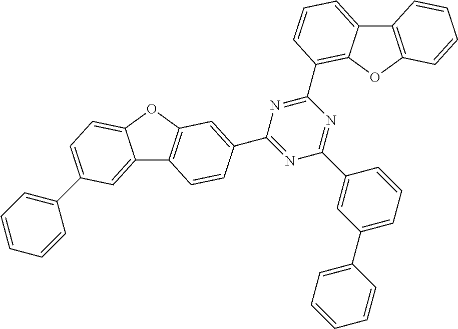 [SMILES: c1ccc(-c2cccc(-c3nc(-c4ccc5c(c4)oc4ccc(-c6ccccc6)cc45)nc(-c4cccc5c4oc4ccccc45)n3)c2)cc1] | 83 |
| 1-11 | 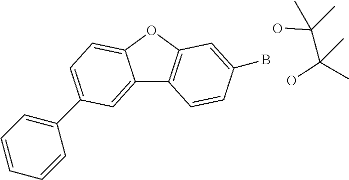 | 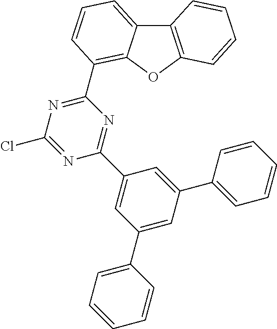 [SMILES: Clc1nc(-c2cc(-c3ccccc3)cc(-c3ccccc3)c2)nc(-c2cccc3c2oc2ccccc23)n1] | 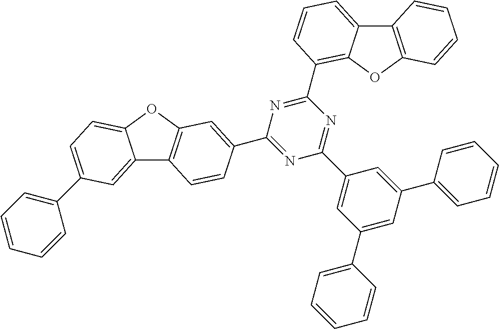 [SMILES: c1ccc(-c2cc(-c3ccccc3)cc(-c3nc(-c4ccc5c(c4)oc4ccc(-c6ccccc6)cc45)nc(-c4cccc5c4oc4ccccc45)n3)c2)cc1] | 84 |
| 1-12 | 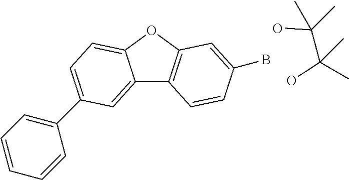 | 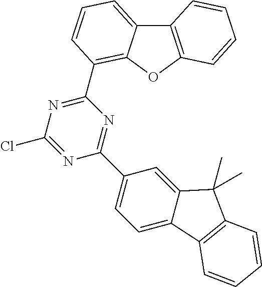 [SMILES: CC1(C)c2ccccc2-c2ccc(-c3nc(Cl)nc(-c4cccc5c4oc4ccccc45)n3)cc21] | 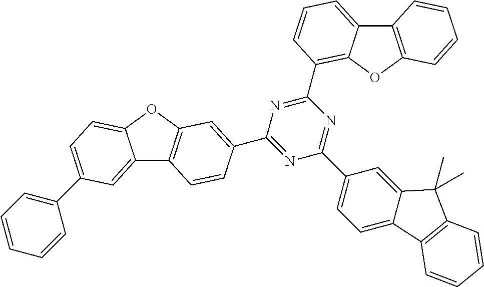 [SMILES: CC1(C)c2ccccc2-c2ccc(-c3nc(-c4ccc5c(c4)oc4ccc(-c6ccccc6)cc45)nc(-c4cccc5c4oc4ccccc45)n3)cc21] | 83 |
| 1-13 | 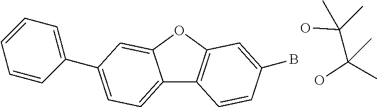 | 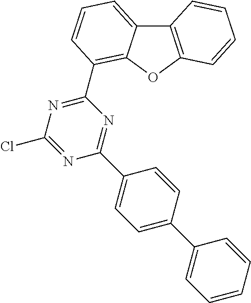 [SMILES: Clc1nc(-c2ccc(-c3ccccc3)cc2)nc(-c2cccc3c2oc2ccccc23)n1] | 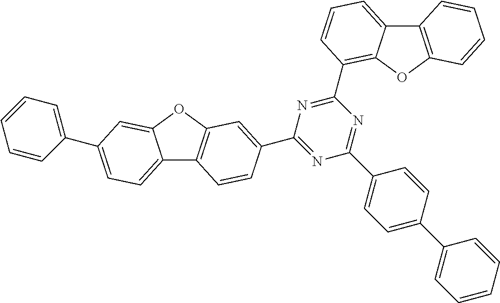 [SMILES: c1ccc(-c2ccc(-c3nc(-c4ccc5c(c4)oc4cc(-c6ccccc6)ccc45)nc(-c4cccc5c4oc4ccccc45)n3)cc2)cc1] | 80 |
| 1-14 | 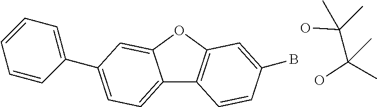 | 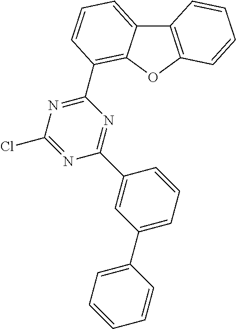 [SMILES: Clc1nc(-c2cccc(-c3ccccc3)c2)nc(-c2cccc3c2oc2ccccc23)n1] | 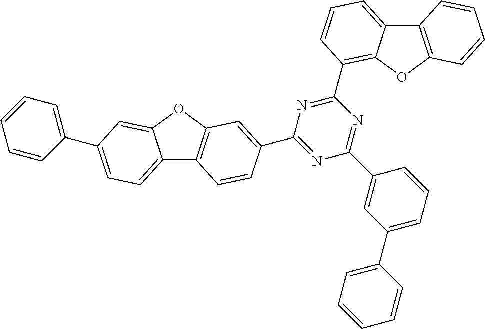 [SMILES: c1ccc(-c2cccc(-c3nc(-c4ccc5c(c4)oc4cc(-c6ccccc6)ccc45)nc(-c4cccc5c4oc4ccccc45)n3)c2)cc1] | 88 |
| 1-15 | 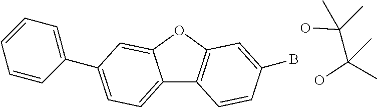 | 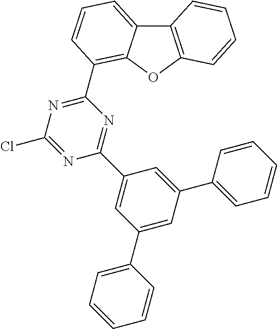 [SMILES: Clc1nc(-c2cc(-c3ccccc3)cc(-c3ccccc3)c2)nc(-c2cccc3c2oc2ccccc23)n1] | 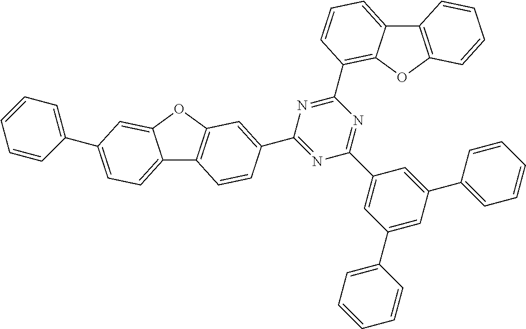 [SMILES: c1ccc(-c2cc(-c3ccccc3)cc(-c3nc(-c4ccc5c(c4)oc4cc(-c6ccccc6)ccc45)nc(-c4cccc5c4oc4ccccc45)n3)c2)cc1] | 86 |
| 1-16 | 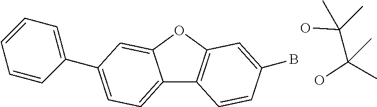 | 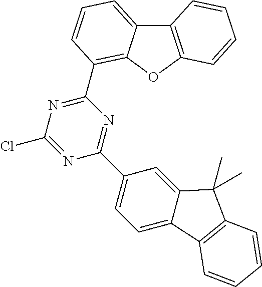 [SMILES: CC1(C)c2ccccc2-c2ccc(-c3nc(Cl)nc(-c4cccc5c4oc4ccccc45)n3)cc21] | 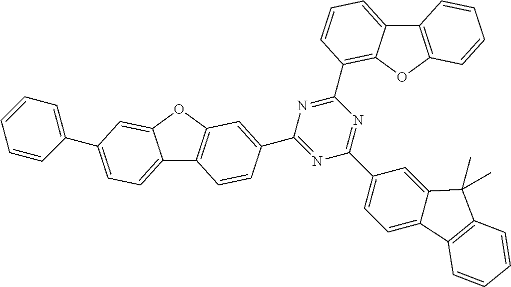 [SMILES: CC1(C)c2ccccc2-c2ccc(-c3nc(-c4ccc5c(c4)oc4cc(-c6ccccc6)ccc45)nc(-c4cccc5c4oc4ccccc45)n3)cc21] | 85 |
| 1-17 | 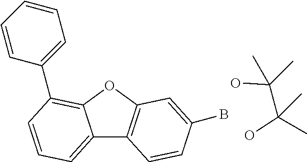 | 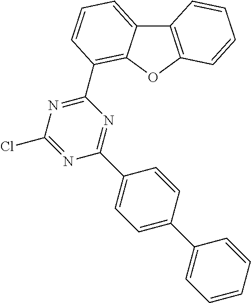 [SMILES: Clc1nc(-c2ccc(-c3ccccc3)cc2)nc(-c2cccc3c2oc2ccccc23)n1] | 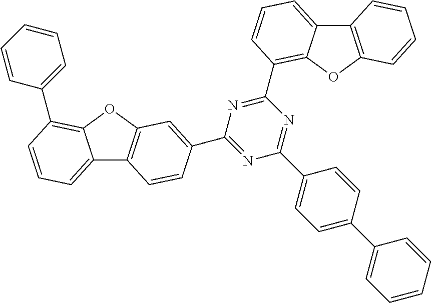 [SMILES: c1ccc(-c2ccc(-c3nc(-c4ccc5c(c4)oc4c(-c6ccccc6)cccc45)nc(-c4cccc5c4oc4ccccc45)n3)cc2)cc1] | 83 |
| 1-18 | 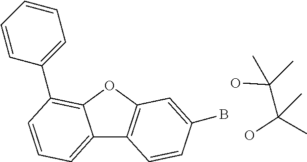 | 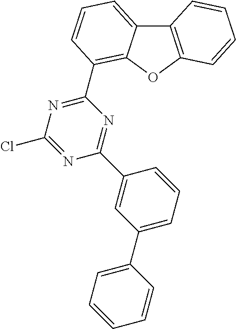 [SMILES: Clc1nc(-c2cccc(-c3ccccc3)c2)nc(-c2cccc3c2oc2ccccc23)n1] | 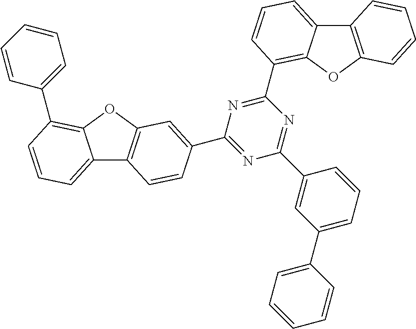 [SMILES: c1ccc(-c2cccc(-c3nc(-c4ccc5c(c4)oc4c(-c6ccccc6)cccc45)nc(-c4cccc5c4oc4ccccc45)n3)c2)cc1] | 84 |
| 1-19 | 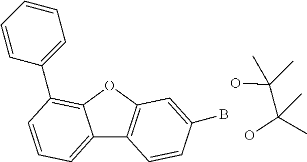 | 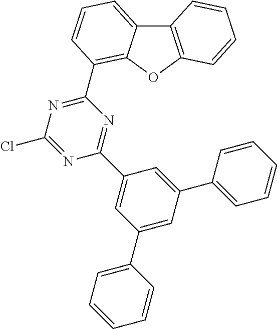 [SMILES: Clc1nc(-c2cc(-c3ccccc3)cc(-c3ccccc3)c2)nc(-c2cccc3c2oc2ccccc23)n1] | 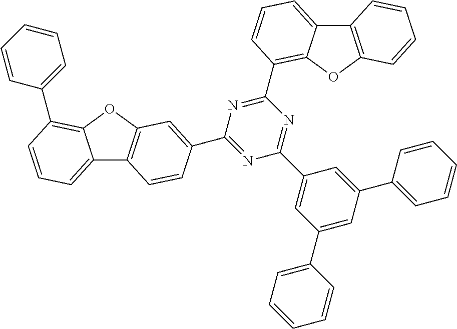 [SMILES: c1ccc(-c2cc(-c3ccccc3)cc(-c3nc(-c4ccc5c(c4)oc4c(-c6ccccc6)cccc45)nc(-c4cccc5c4oc4ccccc45)n3)c2)cc1] | 88 |
| 1-20 | 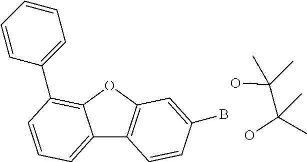 | 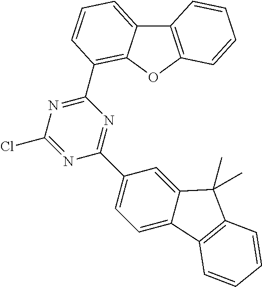 [SMILES: CC1(C)c2ccccc2-c2ccc(-c3nc(Cl)nc(-c4cccc5c4oc4ccccc45)n3)cc21] | 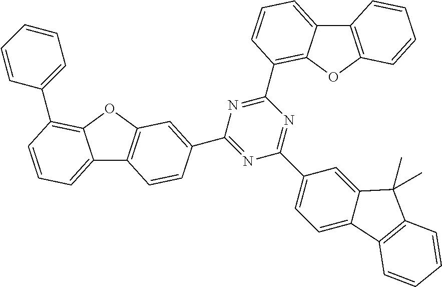 [SMILES: CC1(C)c2ccccc2-c2ccc(-c3nc(-c4ccc5c(c4)oc4c(-c6ccccc6)cccc45)nc(-c4cccc5c4oc4ccccc45)n3)cc21] | 86 |
| 1-21 | 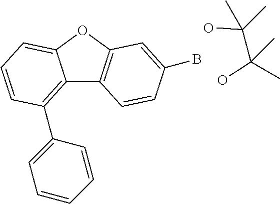 | 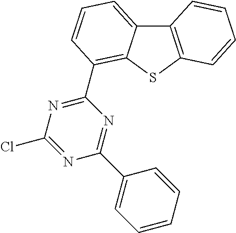 [SMILES: Clc1nc(-c2ccccc2)nc(-c2cccc3c2sc2ccccc23)n1] | 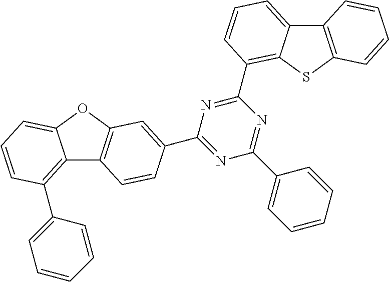 [SMILES: c1ccc(-c2nc(-c3ccc4c(c3)oc3cccc(-c5ccccc5)c34)nc(-c3cccc4c3sc3ccccc34)n2)cc1] | 84 |
| 1-22 | 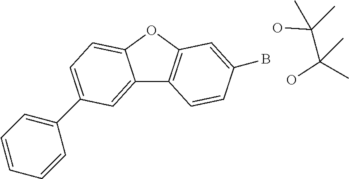 | 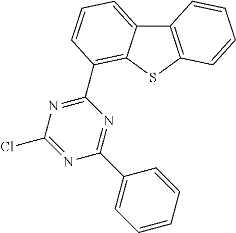 [SMILES: Clc1nc(-c2ccccc2)nc(-c2cccc3c2sc2ccccc23)n1] | 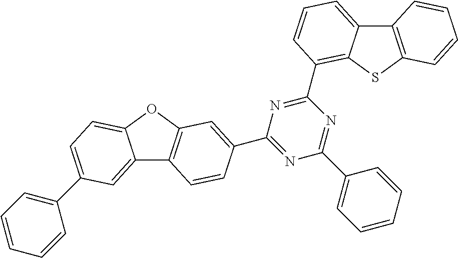 [SMILES: c1ccc(-c2ccc3oc4cc(-c5nc(-c6ccccc6)nc(-c6cccc7c6sc6ccccc67)n5)ccc4c3c2)cc1] | 85 |
| 1-23 | 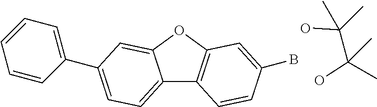 | 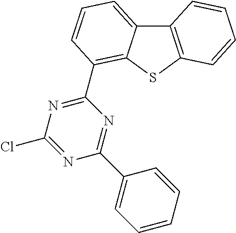 [SMILES: Clc1nc(-c2ccccc2)nc(-c2cccc3c2sc2ccccc23)n1] | 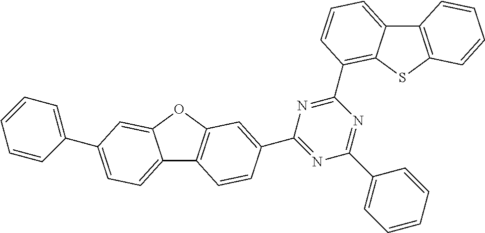 [SMILES: c1ccc(-c2ccc3c(c2)oc2cc(-c4nc(-c5ccccc5)nc(-c5cccc6c5sc5ccccc56)n4)ccc23)cc1] | 83 |
| 1-24 | 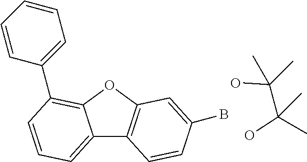 | 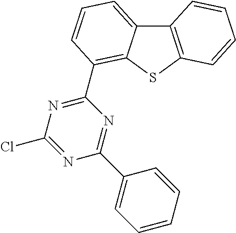 [SMILES: Clc1nc(-c2ccccc2)nc(-c2cccc3c2sc2ccccc23)n1] | 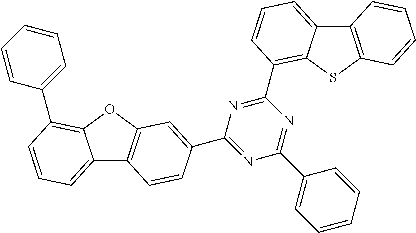 [SMILES: c1ccc(-c2nc(-c3ccc4c(c3)oc3c(-c5ccccc5)cccc34)nc(-c3cccc4c3sc3ccccc34)n2)cc1] | 85 |
| 1-25 | 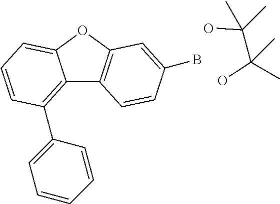 | 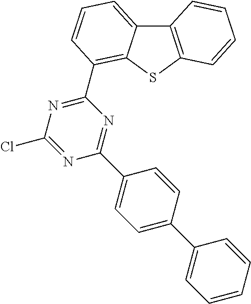 [SMILES: Clc1nc(-c2ccc(-c3ccccc3)cc2)nc(-c2cccc3c2sc2ccccc23)n1] | 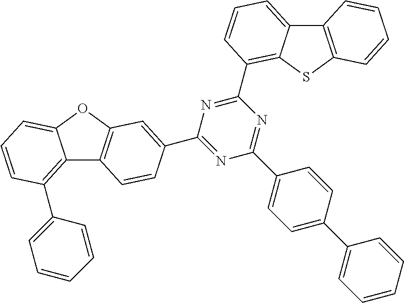 [SMILES: c1ccc(-c2ccc(-c3nc(-c4ccc5c(c4)oc4cccc(-c6ccccc6)c45)nc(-c4cccc5c4sc4ccccc45)n3)cc2)cc1] | 83 |
| 1-26 | 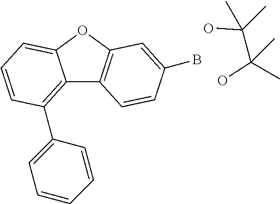 | 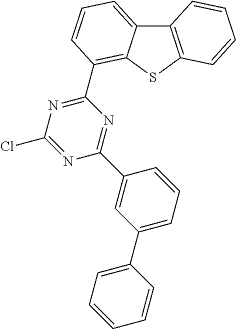 [SMILES: Clc1nc(-c2cccc(-c3ccccc3)c2)nc(-c2cccc3c2sc2ccccc23)n1] | 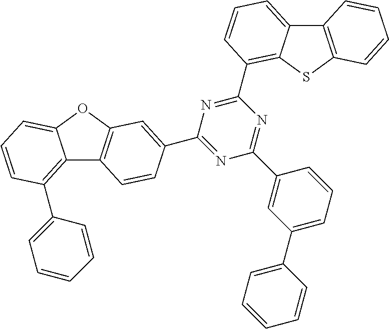 [SMILES: c1ccc(-c2cccc(-c3nc(-c4ccc5c(c4)oc4cccc(-c6ccccc6)c45)nc(-c4cccc5c4sc4ccccc45)n3)c2)cc1] | 80 |
| 1-27 | 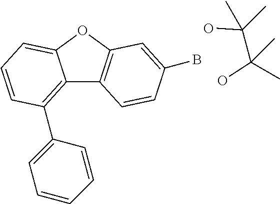 | 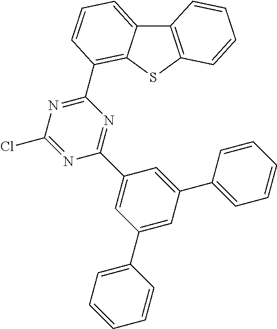 [SMILES: Clc1nc(-c2cc(-c3ccccc3)cc(-c3ccccc3)c2)nc(-c2cccc3c2sc2ccccc23)n1] | 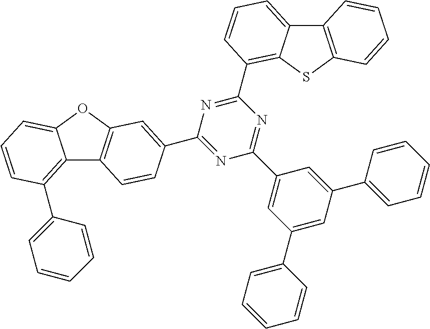 [SMILES: c1ccc(-c2cc(-c3ccccc3)cc(-c3nc(-c4ccc5c(c4)oc4cccc(-c6ccccc6)c45)nc(-c4cccc5c4sc4ccccc45)n3)c2)cc1] | 81 |
| 1-28 | 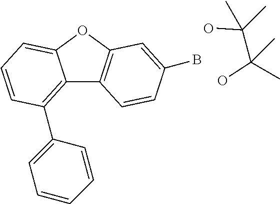 | 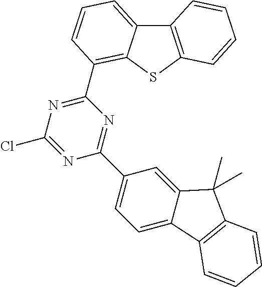 [SMILES: CC1(C)c2ccccc2-c2ccc(-c3nc(Cl)nc(-c4cccc5c4sc4ccccc45)n3)cc21] | 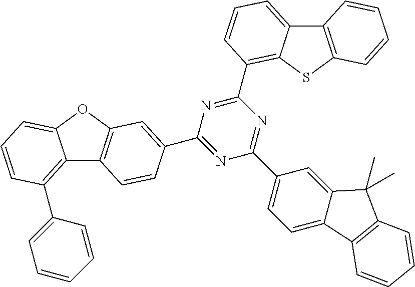 [SMILES: CC1(C)c2ccccc2-c2ccc(-c3nc(-c4ccc5c(c4)oc4cccc(-c6ccccc6)c45)nc(-c4cccc5c4sc4ccccc45)n3)cc21] | 82 |
| 1-29 | 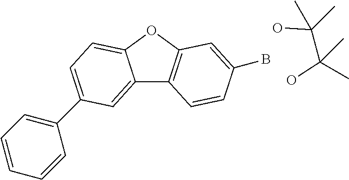 | 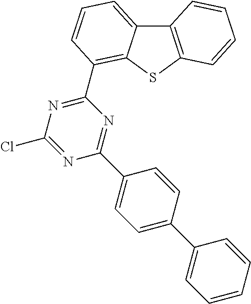 [SMILES: Clc1nc(-c2ccc(-c3ccccc3)cc2)nc(-c2cccc3c2sc2ccccc23)n1] | 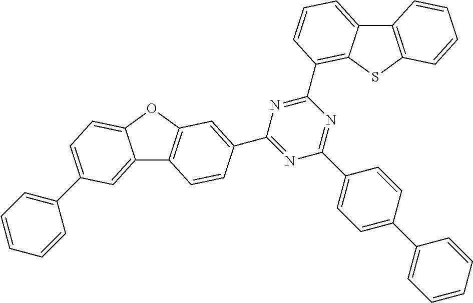 [SMILES: c1ccc(-c2ccc(-c3nc(-c4ccc5c(c4)oc4ccc(-c6ccccc6)cc45)nc(-c4cccc5c4sc4ccccc45)n3)cc2)cc1] | 83 |
| 1-30 | 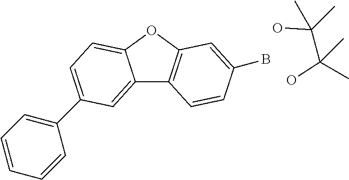 | 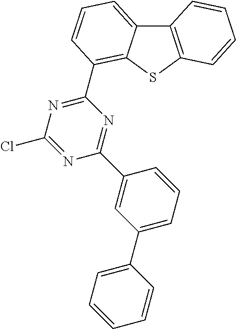 [SMILES: Clc1nc(-c2cccc(-c3ccccc3)c2)nc(-c2cccc3c2sc2ccccc23)n1] | 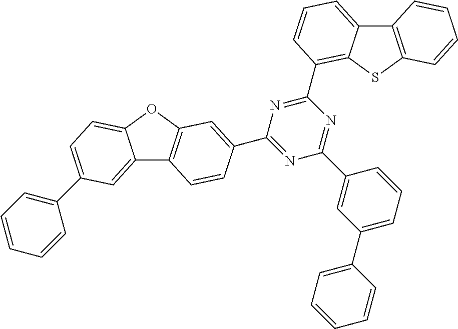 [SMILES: c1ccc(-c2cccc(-c3nc(-c4ccc5c(c4)oc4ccc(-c6ccccc6)cc45)nc(-c4cccc5c4sc4ccccc45)n3)c2)cc1] | 88 |
| 1-31 | 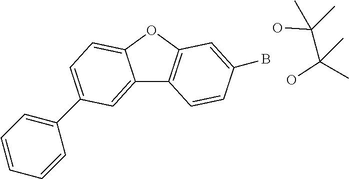 | 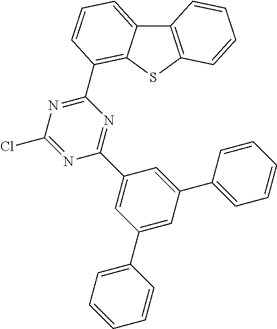 [SMILES: Clc1nc(-c2cc(-c3ccccc3)cc(-c3ccccc3)c2)nc(-c2cccc3c2sc2ccccc23)n1] | 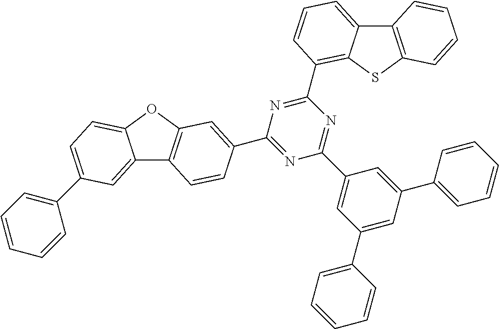 [SMILES: c1ccc(-c2cc(-c3ccccc3)cc(-c3nc(-c4ccc5c(c4)oc4ccc(-c6ccccc6)cc45)nc(-c4cccc5c4sc4ccccc45)n3)c2)cc1] | 85 |
| 1-32 | 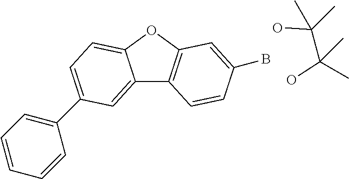 | 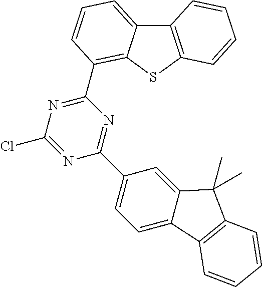 [SMILES: CC1(C)c2ccccc2-c2ccc(-c3nc(Cl)nc(-c4cccc5c4sc4ccccc45)n3)cc21] | 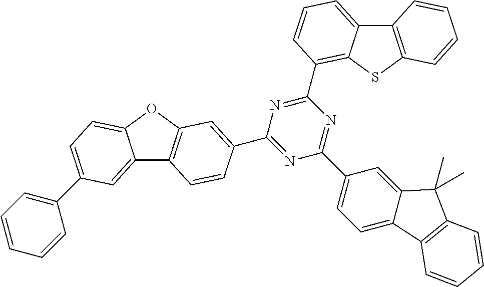 [SMILES: CC1(C)c2ccccc2-c2ccc(-c3nc(-c4ccc5c(c4)oc4ccc(-c6ccccc6)cc45)nc(-c4cccc5c4sc4ccccc45)n3)cc21] | 83 |
| 1-33 | 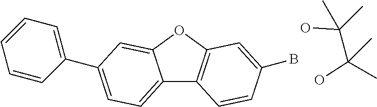 | 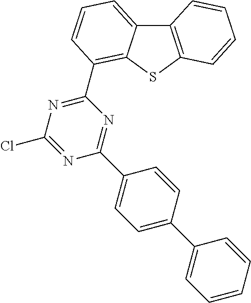 [SMILES: Clc1nc(-c2ccc(-c3ccccc3)cc2)nc(-c2cccc3c2sc2ccccc23)n1] | 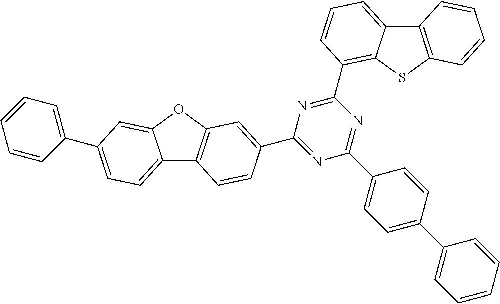 [SMILES: c1ccc(-c2ccc(-c3nc(-c4ccc5c(c4)oc4cc(-c6ccccc6)ccc45)nc(-c4cccc5c4sc4ccccc45)n3)cc2)cc1] | 84 |
| 1-34 | 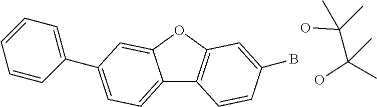 | 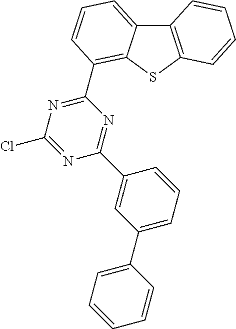 [SMILES: Clc1nc(-c2cccc(-c3ccccc3)c2)nc(-c2cccc3c2sc2ccccc23)n1] | 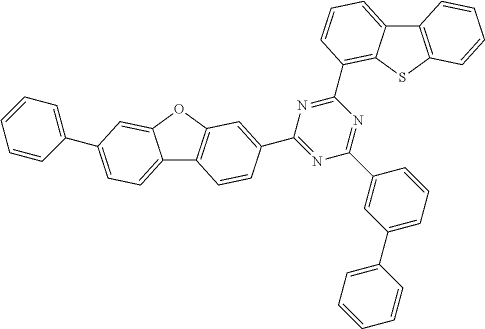 [SMILES: c1ccc(-c2cccc(-c3nc(-c4ccc5c(c4)oc4cc(-c6ccccc6)ccc45)nc(-c4cccc5c4sc4ccccc45)n3)c2)cc1] | 83 |
| 1-35 | 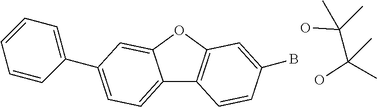 | 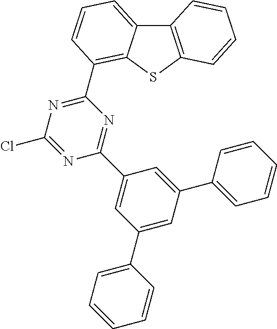 [SMILES: Clc1nc(-c2cc(-c3ccccc3)cc(-c3ccccc3)c2)nc(-c2cccc3c2sc2ccccc23)n1] | 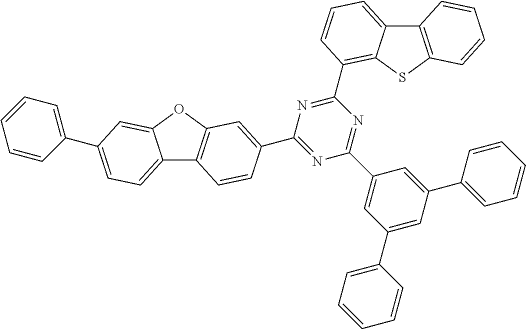 [SMILES: c1ccc(-c2cc(-c3ccccc3)cc(-c3nc(-c4ccc5c(c4)oc4cc(-c6ccccc6)ccc45)nc(-c4cccc5c4sc4ccccc45)n3)c2)cc1] | 80 |
| 1-36 | 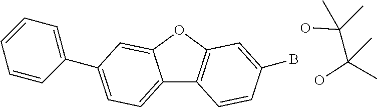 | 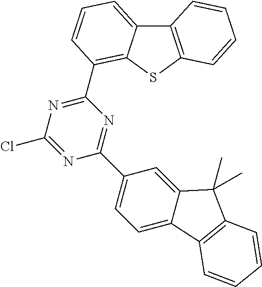 [SMILES: CC1(C)c2ccccc2-c2ccc(-c3nc(Cl)nc(-c4cccc5c4sc4ccccc45)n3)cc21] | 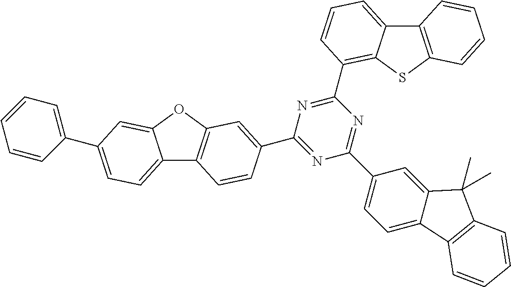 [SMILES: CC1(C)c2ccccc2-c2ccc(-c3nc(-c4ccc5c(c4)oc4cc(-c6ccccc6)ccc45)nc(-c4cccc5c4sc4ccccc45)n3)cc21] | 88 |
| 1-37 | 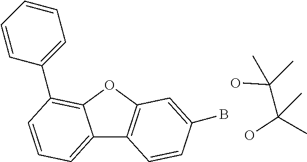 | 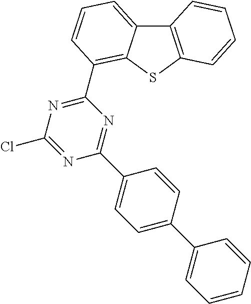 [SMILES: Clc1nc(-c2ccc(-c3ccccc3)cc2)nc(-c2cccc3c2sc2ccccc23)n1] | 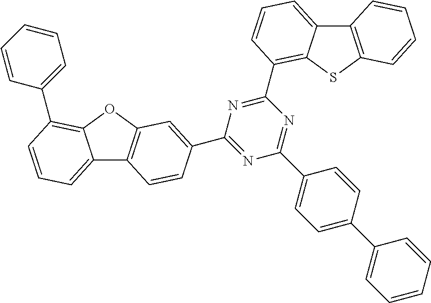 [SMILES: c1ccc(-c2ccc(-c3nc(-c4ccc5c(c4)oc4c(-c6ccccc6)cccc45)nc(-c4cccc5c4sc4ccccc45)n3)cc2)cc1] | 86 |
| 1-38 | 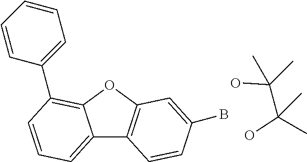 | 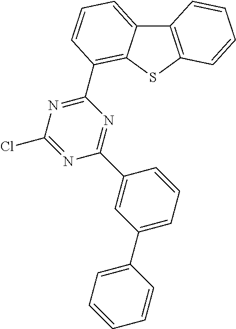 [SMILES: Clc1nc(-c2cccc(-c3ccccc3)c2)nc(-c2cccc3c2sc2ccccc23)n1] | 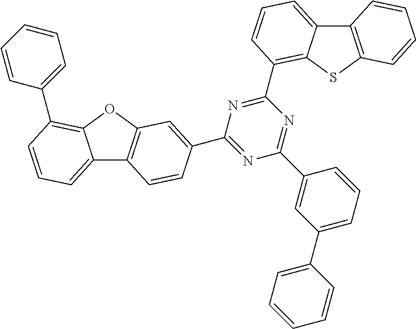 [SMILES: c1ccc(-c2cccc(-c3nc(-c4ccc5c(c4)oc4c(-c6ccccc6)cccc45)nc(-c4cccc5c4sc4ccccc45)n3)c2)cc1] | 85 |
| 1-39 | 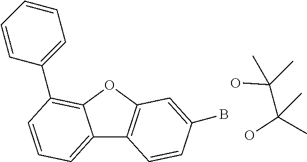 | 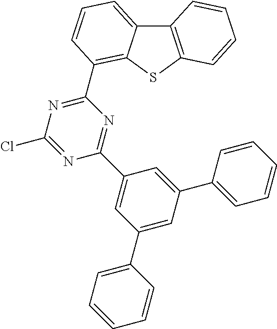 [SMILES: Clc1nc(-c2cc(-c3ccccc3)cc(-c3ccccc3)c2)nc(-c2cccc3c2sc2ccccc23)n1] | 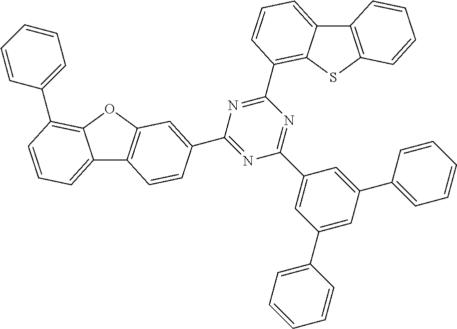 [SMILES: c1ccc(-c2cc(-c3ccccc3)cc(-c3nc(-c4ccc5c(c4)oc4c(-c6ccccc6)cccc45)nc(-c4cccc5c4sc4ccccc45)n3)c2)cc1] | 83 |
| 1-40 | 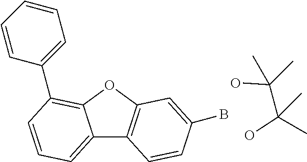 | 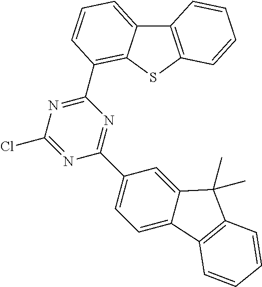 [SMILES: CC1(C)c2ccccc2-c2ccc(-c3nc(Cl)nc(-c4cccc5c4sc4ccccc45)n3)cc21] | 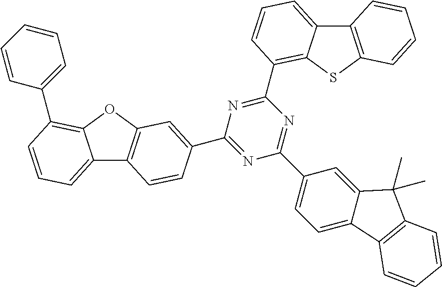 [SMILES: CC1(C)c2ccccc2-c2ccc(-c3nc(-c4ccc5c(c4)oc4c(-c6ccccc6)cccc45)nc(-c4cccc5c4sc4ccccc45)n3)cc21] | 84 |
| 1-41 | 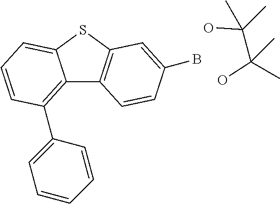 | 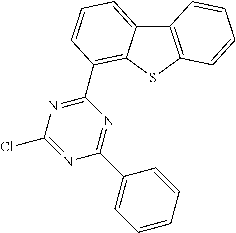 [SMILES: Clc1nc(-c2ccccc2)nc(-c2cccc3c2sc2ccccc23)n1] | 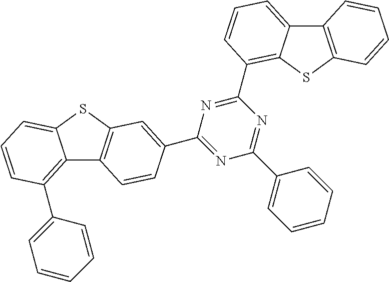 [SMILES: c1ccc(-c2nc(-c3ccc4c(c3)sc3cccc(-c5ccccc5)c34)nc(-c3cccc4c3sc3ccccc34)n2)cc1] | 88 |
| 1-42 | 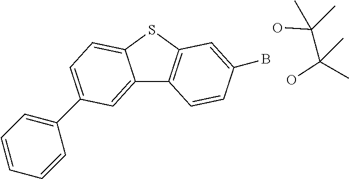 | 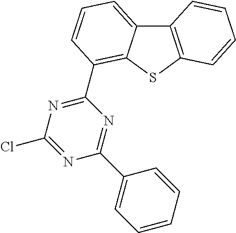 [SMILES: Clc1nc(-c2ccccc2)nc(-c2cccc3c2sc2ccccc23)n1] | 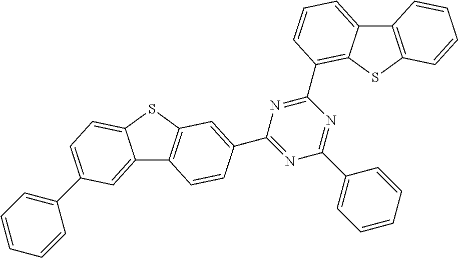 [SMILES: c1ccc(-c2ccc3sc4cc(-c5nc(-c6ccccc6)nc(-c6cccc7c6sc6ccccc67)n5)ccc4c3c2)cc1] | 86 |
| 1-43 | 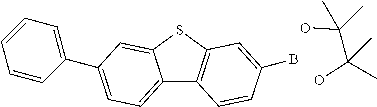 | 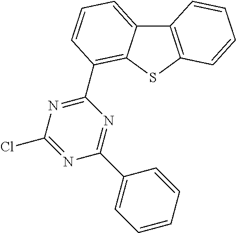 [SMILES: Clc1nc(-c2ccccc2)nc(-c2cccc3c2sc2ccccc23)n1] | 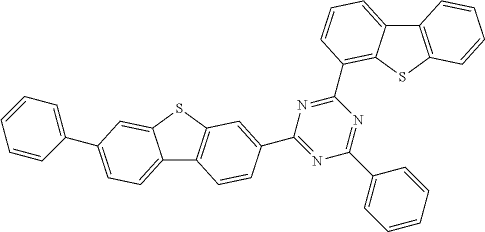 [SMILES: c1ccc(-c2ccc3c(c2)sc2cc(-c4nc(-c5ccccc5)nc(-c5cccc6c5sc5ccccc56)n4)ccc23)cc1] | 84 |
| 1-44 | 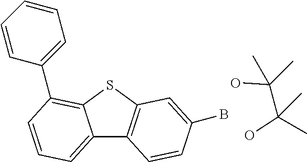 | 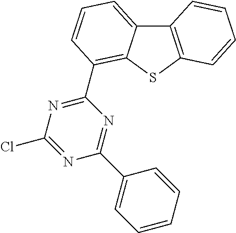 [SMILES: Clc1nc(-c2ccccc2)nc(-c2cccc3c2sc2ccccc23)n1] | 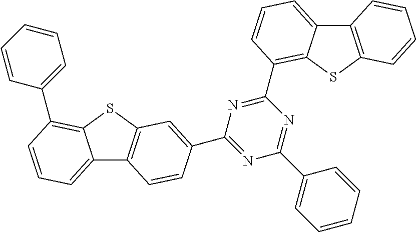 [SMILES: c1ccc(-c2nc(-c3ccc4c(c3)sc3c(-c5ccccc5)cccc34)nc(-c3cccc4c3sc3ccccc34)n2)cc1] | 85 |
| 1-45 | 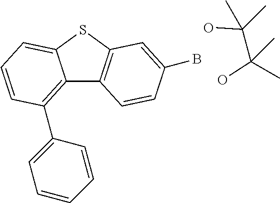 | 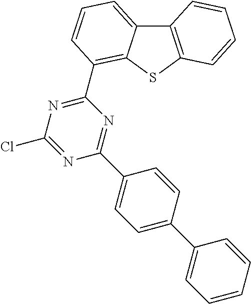 [SMILES: Clc1nc(-c2ccc(-c3ccccc3)cc2)nc(-c2cccc3c2sc2ccccc23)n1] | 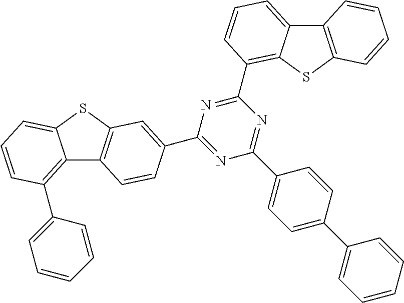 [SMILES: c1ccc(-c2ccc(-c3nc(-c4ccc5c(c4)sc4cccc(-c6ccccc6)c45)nc(-c4cccc5c4sc4ccccc45)n3)cc2)cc1] | 83 |
| 1-46 | 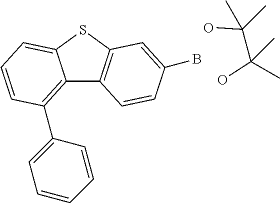 | 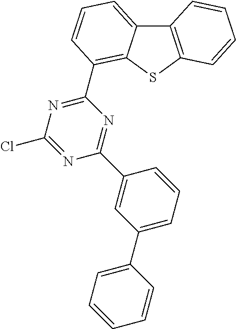 [SMILES: Clc1nc(-c2cccc(-c3ccccc3)c2)nc(-c2cccc3c2sc2ccccc23)n1] | 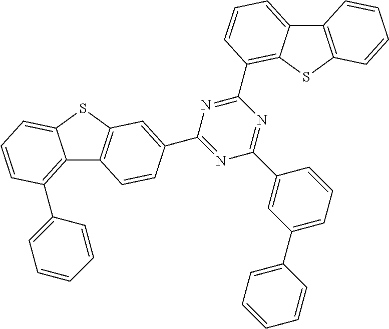 [SMILES: c1ccc(-c2cccc(-c3nc(-c4ccc5c(c4)sc4cccc(-c6ccccc6)c45)nc(-c4cccc5c4sc4ccccc45)n3)c2)cc1] | 85 |
| 1-47 | 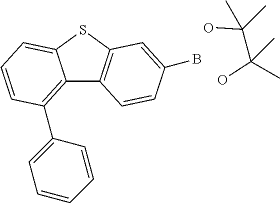 | 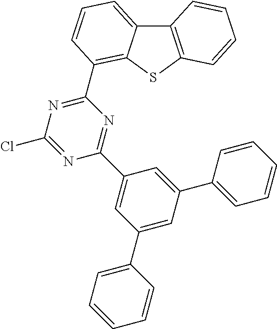 [SMILES: Clc1nc(-c2cc(-c3ccccc3)cc(-c3ccccc3)c2)nc(-c2cccc3c2sc2ccccc23)n1] | 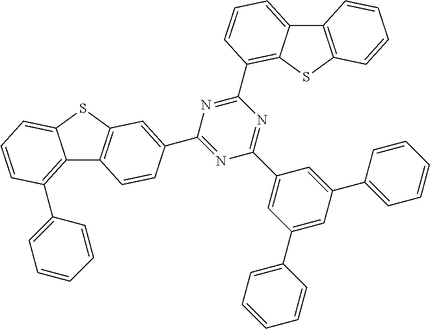 [SMILES: c1ccc(-c2cc(-c3ccccc3)cc(-c3nc(-c4ccc5c(c4)sc4cccc(-c6ccccc6)c45)nc(-c4cccc5c4sc4ccccc45)n3)c2)cc1] | 83 |
| 1-48 | 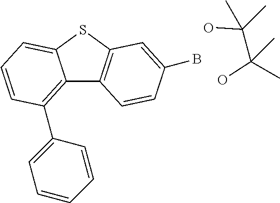 | 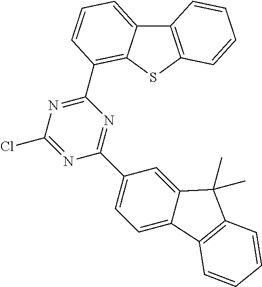 [SMILES: CC1(C)c2ccccc2-c2ccc(-c3nc(Cl)nc(-c4cccc5c4sc4ccccc45)n3)cc21] | 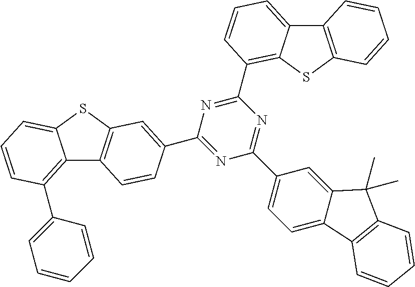 [SMILES: CC1(C)c2ccccc2-c2ccc(-c3nc(-c4ccc5c(c4)sc4cccc(-c6ccccc6)c45)nc(-c4cccc5c4sc4ccccc45)n3)cc21] | 80 |
| 1-49 | 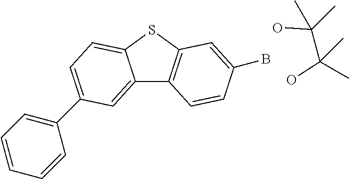 | 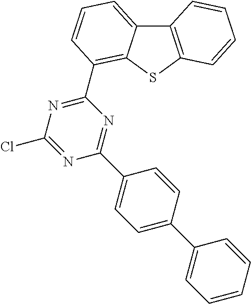 [SMILES: Clc1nc(-c2ccc(-c3ccccc3)cc2)nc(-c2cccc3c2sc2ccccc23)n1] | 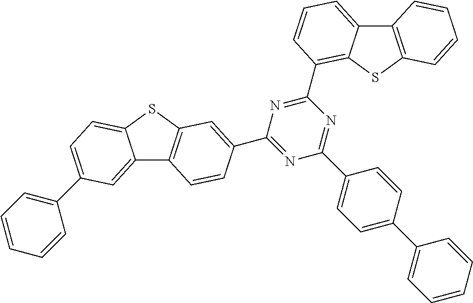 [SMILES: c1ccc(-c2ccc(-c3nc(-c4ccc5c(c4)sc4ccc(-c6ccccc6)cc45)nc(-c4cccc5c4sc4ccccc45)n3)cc2)cc1] | 81 |
| 1-50 | 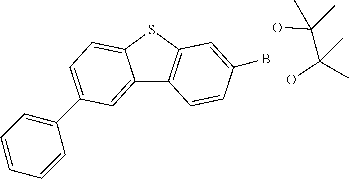 | 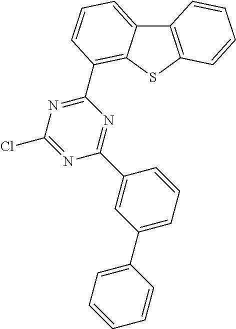 [SMILES: Clc1nc(-c2cccc(-c3ccccc3)c2)nc(-c2cccc3c2sc2ccccc23)n1] | 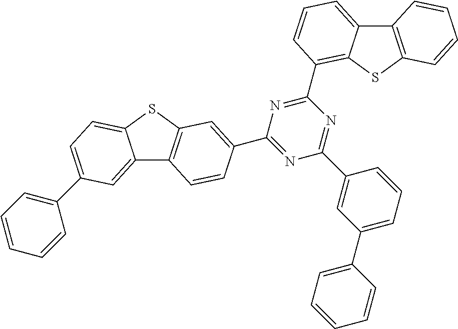 [SMILES: c1ccc(-c2cccc(-c3nc(-c4ccc5c(c4)sc4ccc(-c6ccccc6)cc45)nc(-c4cccc5c4sc4ccccc45)n3)c2)cc1] | 82 |
| 1-51 | 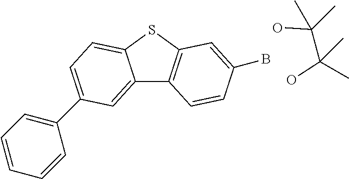 | 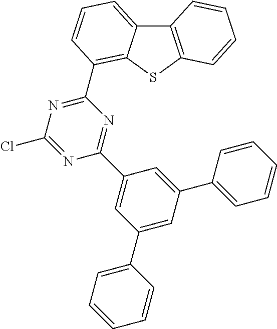 [SMILES: Clc1nc(-c2cc(-c3ccccc3)cc(-c3ccccc3)c2)nc(-c2cccc3c2sc2ccccc23)n1] | 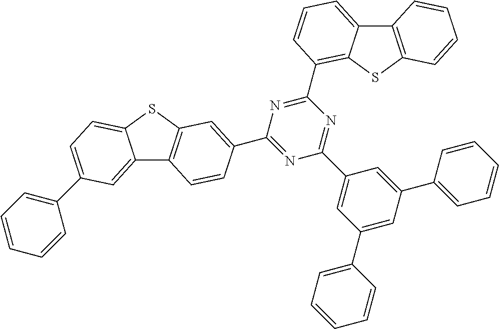 [SMILES: c1ccc(-c2cc(-c3ccccc3)cc(-c3nc(-c4ccc5c(c4)sc4ccc(-c6ccccc6)cc45)nc(-c4cccc5c4sc4ccccc45)n3)c2)cc1] | 83 |
| 1-52 | 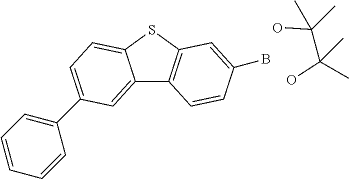 | 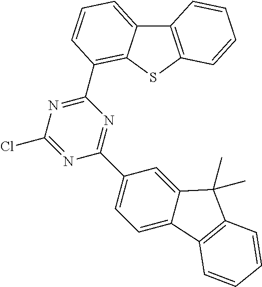 [SMILES: CC1(C)c2ccccc2-c2ccc(-c3nc(Cl)nc(-c4cccc5c4sc4ccccc45)n3)cc21] | 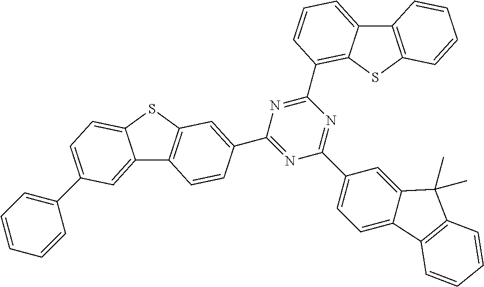 [SMILES: CC1(C)c2ccccc2-c2ccc(-c3nc(-c4ccc5c(c4)sc4ccc(-c6ccccc6)cc45)nc(-c4cccc5c4sc4ccccc45)n3)cc21] | 88 |
| 1-53 | 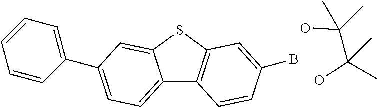 | 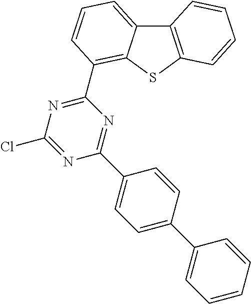 [SMILES: Clc1nc(-c2ccc(-c3ccccc3)cc2)nc(-c2cccc3c2sc2ccccc23)n1] | 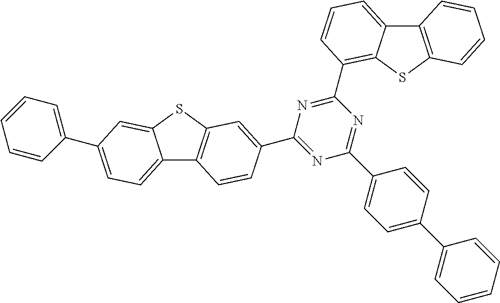 [SMILES: c1ccc(-c2ccc(-c3nc(-c4ccc5c(c4)sc4cc(-c6ccccc6)ccc45)nc(-c4cccc5c4sc4ccccc45)n3)cc2)cc1] | 85 |
| 1-54 | 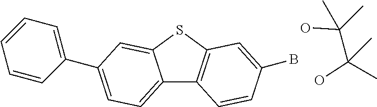 | 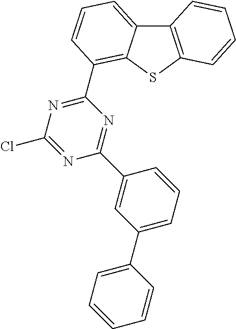 [SMILES: Clc1nc(-c2cccc(-c3ccccc3)c2)nc(-c2cccc3c2sc2ccccc23)n1] | 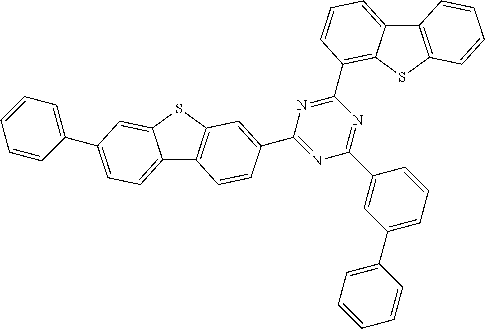 [SMILES: c1ccc(-c2cccc(-c3nc(-c4ccc5c(c4)sc4cc(-c6ccccc6)ccc45)nc(-c4cccc5c4sc4ccccc45)n3)c2)cc1] | 83 |
| 1-55 | 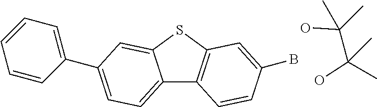 | 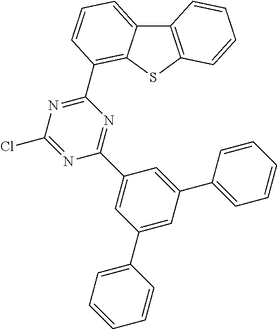 [SMILES: Clc1nc(-c2cc(-c3ccccc3)cc(-c3ccccc3)c2)nc(-c2cccc3c2sc2ccccc23)n1] | 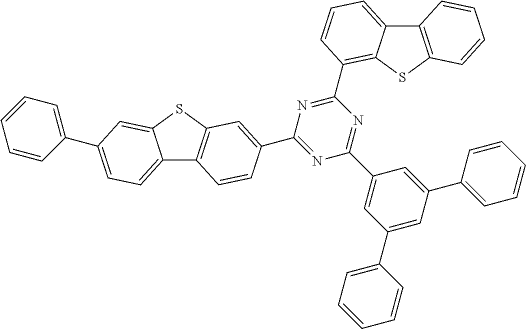 [SMILES: c1ccc(-c2cc(-c3ccccc3)cc(-c3nc(-c4ccc5c(c4)sc4cc(-c6ccccc6)ccc45)nc(-c4cccc5c4sc4ccccc45)n3)c2)cc1] | 84 |
| 1-56 | 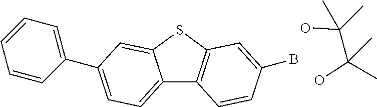 | 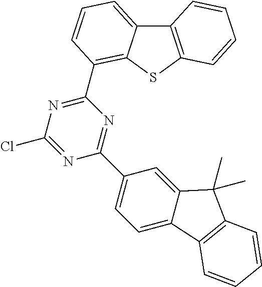 [SMILES: CC1(C)c2ccccc2-c2ccc(-c3nc(Cl)nc(-c4cccc5c4sc4ccccc45)n3)cc21] | 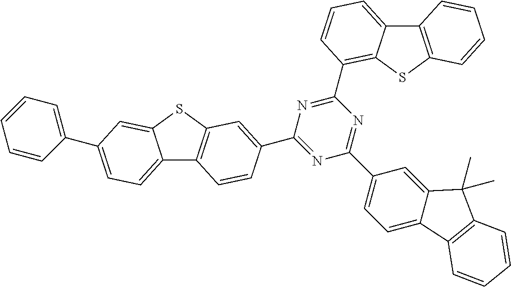 [SMILES: CC1(C)c2ccccc2-c2ccc(-c3nc(-c4ccc5c(c4)sc4cc(-c6ccccc6)ccc45)nc(-c4cccc5c4sc4ccccc45)n3)cc21] | 83 |
| 1-57 | 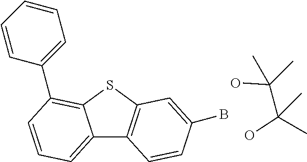 | 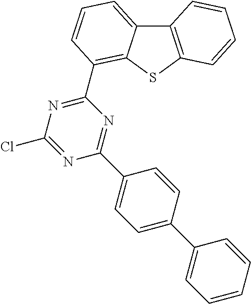 [SMILES: Clc1nc(-c2ccc(-c3ccccc3)cc2)nc(-c2cccc3c2sc2ccccc23)n1] | 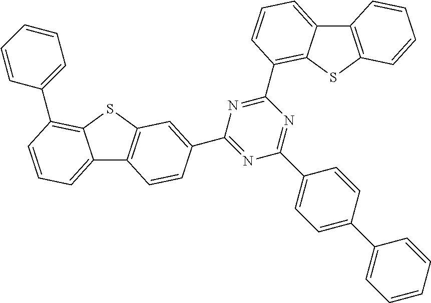 [SMILES: c1ccc(-c2ccc(-c3nc(-c4ccc5c(c4)sc4c(-c6ccccc6)cccc45)nc(-c4cccc5c4sc4ccccc45)n3)cc2)cc1] | 80 |
| 1-58 | 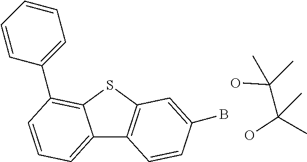 | 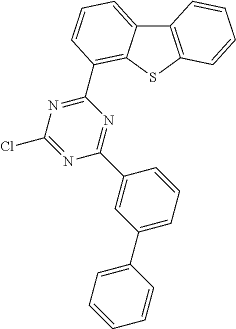 [SMILES: Clc1nc(-c2cccc(-c3ccccc3)c2)nc(-c2cccc3c2sc2ccccc23)n1] | 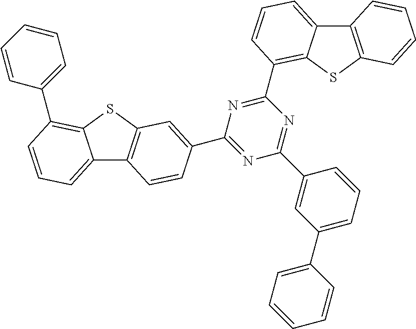 [SMILES: c1ccc(-c2cccc(-c3nc(-c4ccc5c(c4)sc4c(-c6ccccc6)cccc45)nc(-c4cccc5c4sc4ccccc45)n3)c2)cc1] | 88 |
| 1-59 | 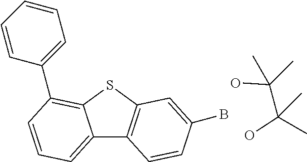 | 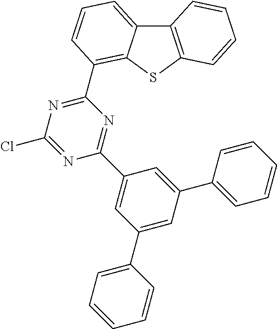 [SMILES: Clc1nc(-c2cc(-c3ccccc3)cc(-c3ccccc3)c2)nc(-c2cccc3c2sc2ccccc23)n1] | 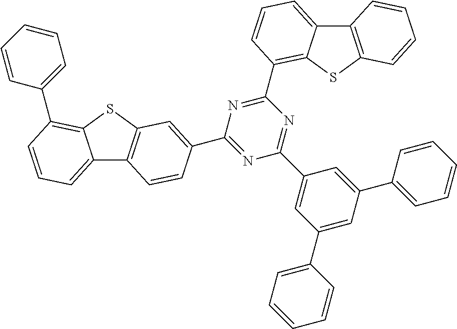 [SMILES: c1ccc(-c2cc(-c3ccccc3)cc(-c3nc(-c4ccc5c(c4)sc4c(-c6ccccc6)cccc45)nc(-c4cccc5c4sc4ccccc45)n3)c2)cc1] | 86 |
| 1-60 | 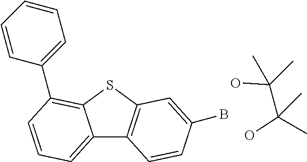 | 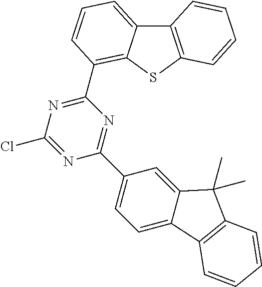 [SMILES: CC1(C)c2ccccc2-c2ccc(-c3nc(Cl)nc(-c4cccc5c4sc4ccccc45)n3)cc21] | 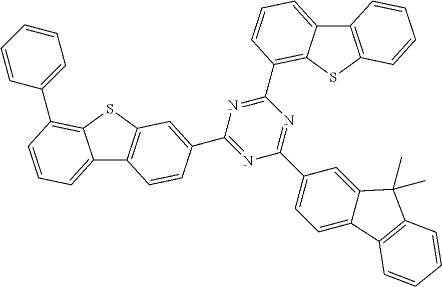 [SMILES: CC1(C)c2ccccc2-c2ccc(-c3nc(-c4ccc5c(c4)sc4c(-c6ccccc6)cccc45)nc(-c4cccc5c4sc4ccccc45)n3)cc21] | 85 |
| 1-61 | 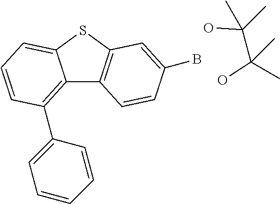 | 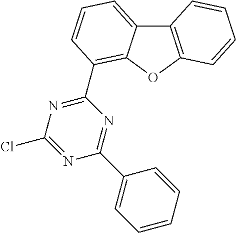 [SMILES: Clc1nc(-c2ccccc2)nc(-c2cccc3c2oc2ccccc23)n1] | 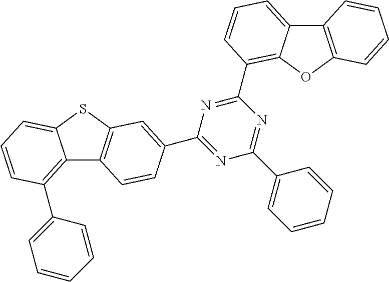 [SMILES: c1ccc(-c2nc(-c3ccc4c(c3)sc3cccc(-c5ccccc5)c34)nc(-c3cccc4c3oc3ccccc34)n2)cc1] | 83 |
| 1-62 | 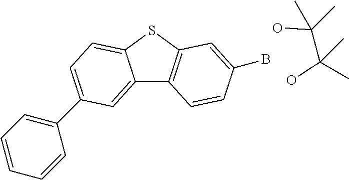 | 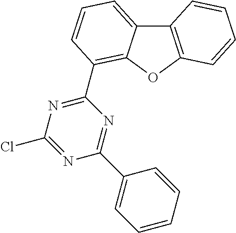 [SMILES: Clc1nc(-c2ccccc2)nc(-c2cccc3c2oc2ccccc23)n1] | 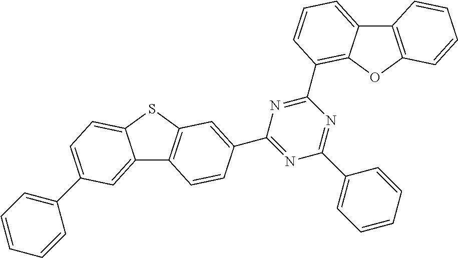 [SMILES: c1ccc(-c2ccc3sc4cc(-c5nc(-c6ccccc6)nc(-c6cccc7c6oc6ccccc67)n5)ccc4c3c2)cc1] | 84 |
| 1-63 | 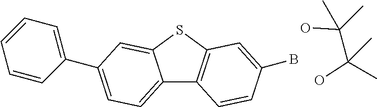 | 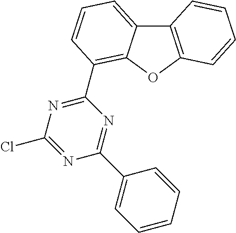 [SMILES: Clc1nc(-c2ccccc2)nc(-c2cccc3c2oc2ccccc23)n1] | 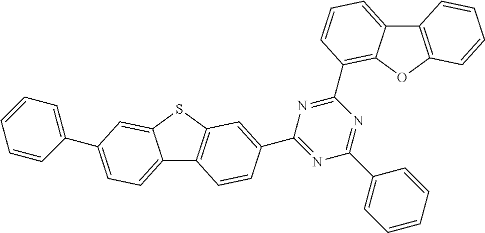 [SMILES: c1ccc(-c2ccc3c(c2)sc2cc(-c4nc(-c5ccccc5)nc(-c5cccc6c5oc5ccccc56)n4)ccc23)cc1] | 88 |
| 1-64 | 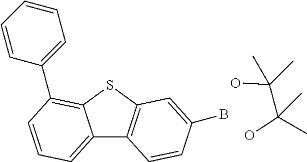 | 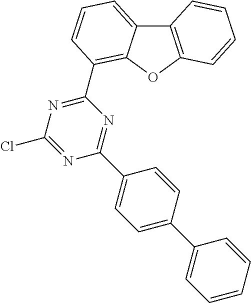 [SMILES: Clc1nc(-c2ccc(-c3ccccc3)cc2)nc(-c2cccc3c2oc2ccccc23)n1] | 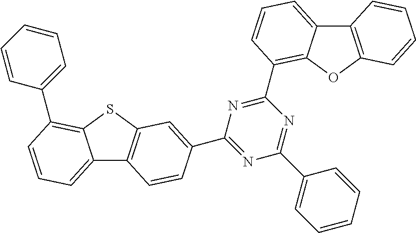 [SMILES: c1ccc(-c2nc(-c3ccc4c(c3)sc3c(-c5ccccc5)cccc34)nc(-c3cccc4c3oc3ccccc34)n2)cc1] | 86 |
| 1-65 | 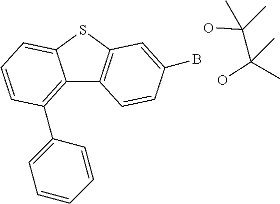 | 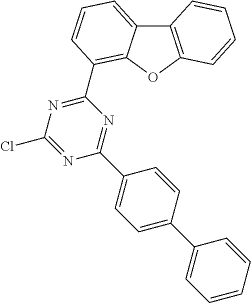 [SMILES: Clc1nc(-c2ccc(-c3ccccc3)cc2)nc(-c2cccc3c2oc2ccccc23)n1] | 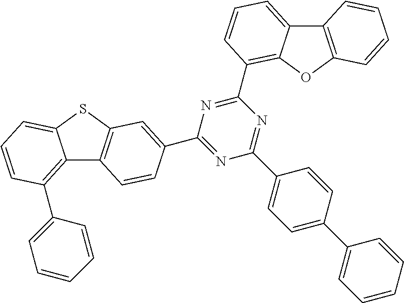 [SMILES: c1ccc(-c2ccc(-c3nc(-c4ccc5c(c4)sc4cccc(-c6ccccc6)c45)nc(-c4cccc5c4oc4ccccc45)n3)cc2)cc1] | 84 |
| 1-66 | 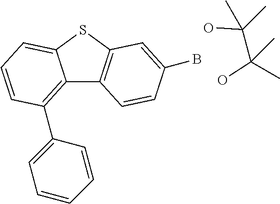 | 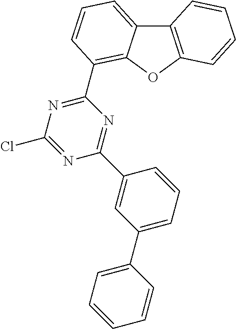 [SMILES: Clc1nc(-c2cccc(-c3ccccc3)c2)nc(-c2cccc3c2oc2ccccc23)n1] | 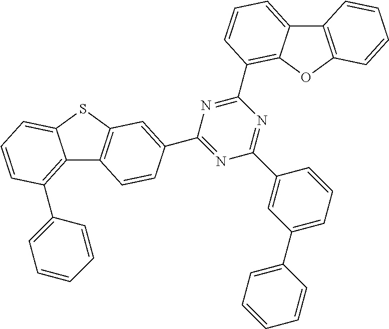 [SMILES: c1ccc(-c2cccc(-c3nc(-c4ccc5c(c4)sc4cccc(-c6ccccc6)c45)nc(-c4cccc5c4oc4ccccc45)n3)c2)cc1] | 85 |
| 1-67 | 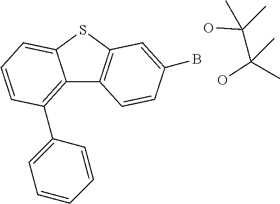 | 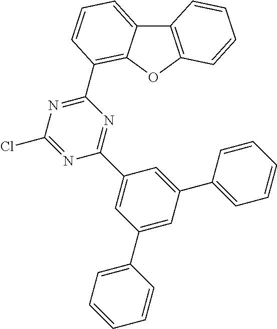 [SMILES: Clc1nc(-c2cc(-c3ccccc3)cc(-c3ccccc3)c2)nc(-c2cccc3c2oc2ccccc23)n1] | 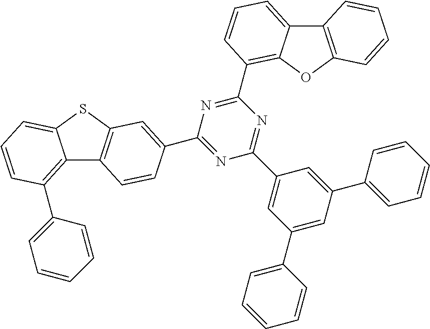 [SMILES: c1ccc(-c2cc(-c3ccccc3)cc(-c3nc(-c4ccc5c(c4)sc4cccc(-c6ccccc6)c45)nc(-c4cccc5c4oc4ccccc45)n3)c2)cc1] | 83 |
| 1-68 | 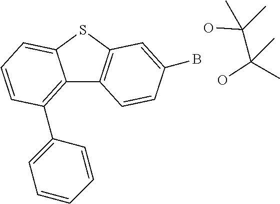 | 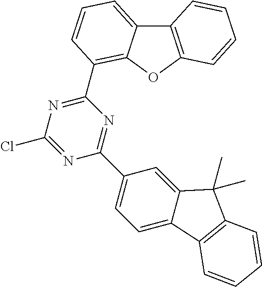 [SMILES: CC1(C)c2ccccc2-c2ccc(-c3nc(Cl)nc(-c4cccc5c4oc4ccccc45)n3)cc21] | 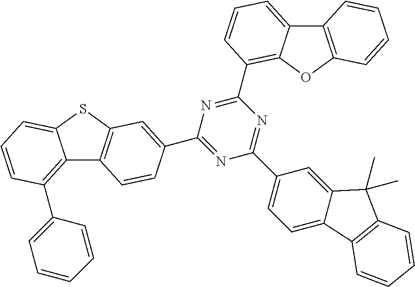 [SMILES: CC1(C)c2ccccc2-c2ccc(-c3nc(-c4ccc5c(c4)sc4cccc(-c6ccccc6)c45)nc(-c4cccc5c4oc4ccccc45)n3)cc21] | 85 |
| 1-69 | 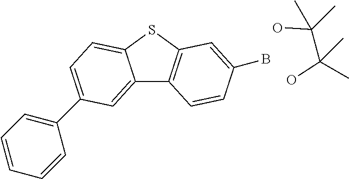 | 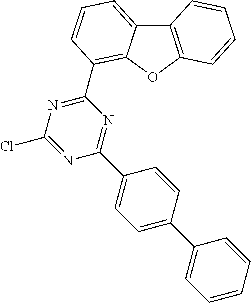 [SMILES: Clc1nc(-c2ccc(-c3ccccc3)cc2)nc(-c2cccc3c2oc2ccccc23)n1] | 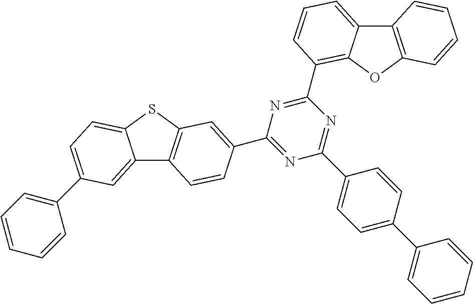 [SMILES: c1ccc(-c2ccc(-c3nc(-c4ccc5c(c4)sc4ccc(-c6ccccc6)cc45)nc(-c4cccc5c4oc4ccccc45)n3)cc2)cc1] | 83 |
| 1-70 | 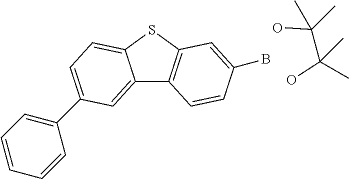 | 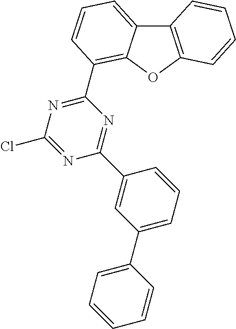 [SMILES: Clc1nc(-c2cccc(-c3ccccc3)c2)nc(-c2cccc3c2oc2ccccc23)n1] | 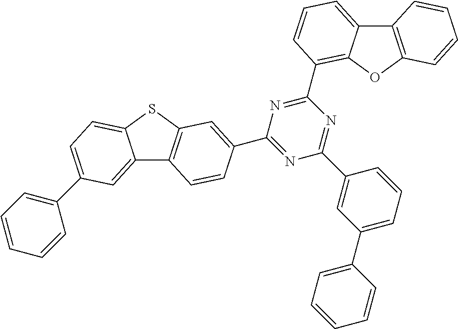 [SMILES: c1ccc(-c2cccc(-c3nc(-c4ccc5c(c4)sc4ccc(-c6ccccc6)cc45)nc(-c4cccc5c4oc4ccccc45)n3)c2)cc1] | 80 |
| 1-71 | 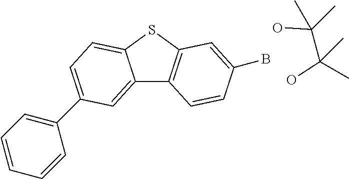 | 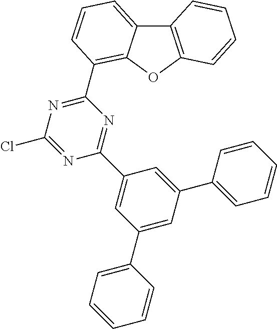 [SMILES: Clc1nc(-c2cc(-c3ccccc3)cc(-c3ccccc3)c2)nc(-c2cccc3c2oc2ccccc23)n1] | 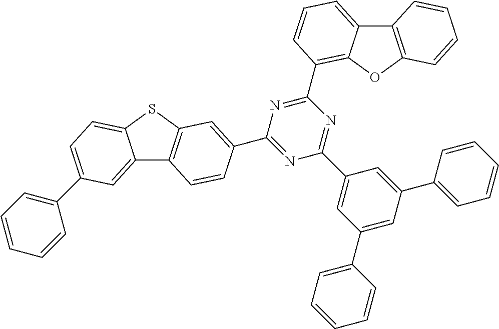 [SMILES: c1ccc(-c2cc(-c3ccccc3)cc(-c3nc(-c4ccc5c(c4)sc4ccc(-c6ccccc6)cc45)nc(-c4cccc5c4oc4ccccc45)n3)c2)cc1] | 81 |
| 1-72 | 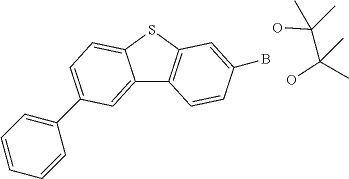 | 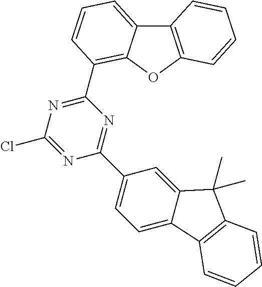 [SMILES: CC1(C)c2ccccc2-c2ccc(-c3nc(Cl)nc(-c4cccc5c4oc4ccccc45)n3)cc21] | 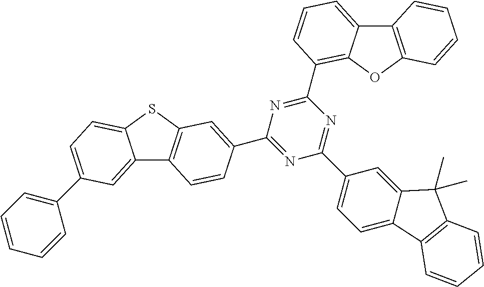 [SMILES: CC1(C)c2ccccc2-c2ccc(-c3nc(-c4ccc5c(c4)sc4ccc(-c6ccccc6)cc45)nc(-c4cccc5c4oc4ccccc45)n3)cc21] | 82 |
| 1-73 | 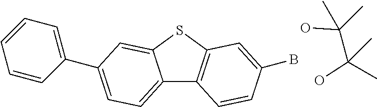 | 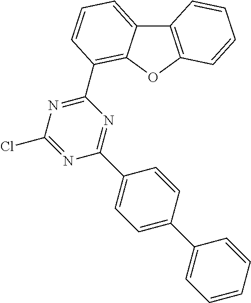 [SMILES: Clc1nc(-c2ccc(-c3ccccc3)cc2)nc(-c2cccc3c2oc2ccccc23)n1] | 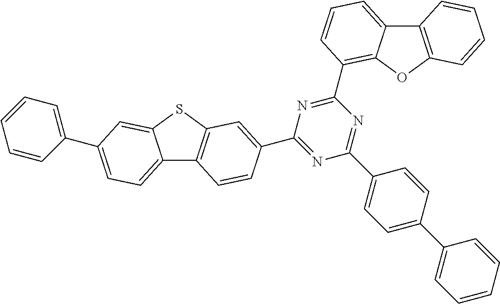 [SMILES: c1ccc(-c2ccc(-c3nc(-c4ccc5c(c4)sc4cc(-c6ccccc6)ccc45)nc(-c4cccc5c4oc4ccccc45)n3)cc2)cc1] | 83 |
| 1-74 | 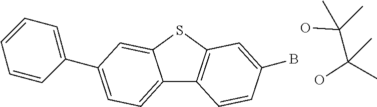 | 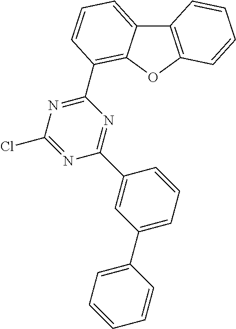 [SMILES: Clc1nc(-c2cccc(-c3ccccc3)c2)nc(-c2cccc3c2oc2ccccc23)n1] | 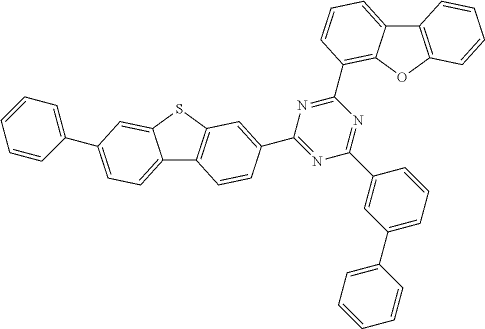 [SMILES: c1ccc(-c2cccc(-c3nc(-c4ccc5c(c4)sc4cc(-c6ccccc6)ccc45)nc(-c4cccc5c4oc4ccccc45)n3)c2)cc1] | 88 |
| 1-75 | 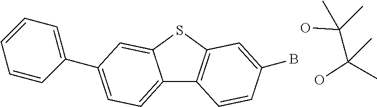 | 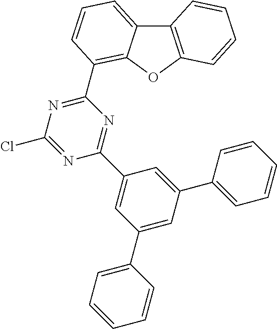 [SMILES: Clc1nc(-c2cc(-c3ccccc3)cc(-c3ccccc3)c2)nc(-c2cccc3c2oc2ccccc23)n1] | 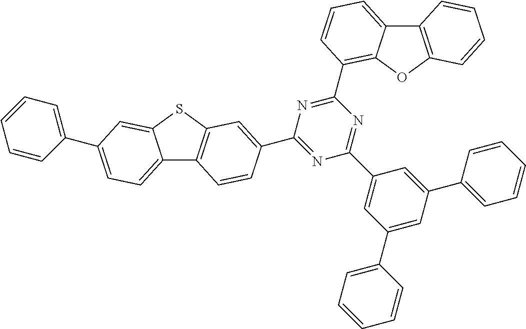 [SMILES: c1ccc(-c2cc(-c3ccccc3)cc(-c3nc(-c4ccc5c(c4)sc4cc(-c6ccccc6)ccc45)nc(-c4cccc5c4oc4ccccc45)n3)c2)cc1] | 85 |
| 1-76 | 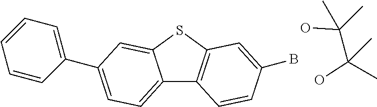 | 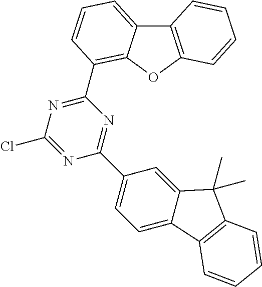 [SMILES: CC1(C)c2ccccc2-c2ccc(-c3nc(Cl)nc(-c4cccc5c4oc4ccccc45)n3)cc21] | 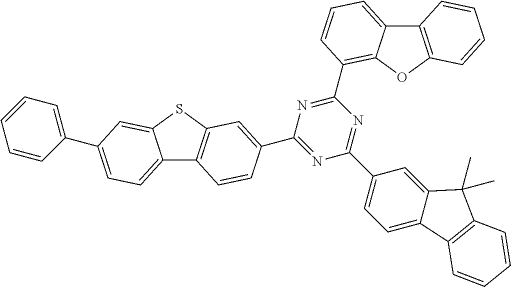 [SMILES: CC1(C)c2ccccc2-c2ccc(-c3nc(-c4ccc5c(c4)sc4cc(-c6ccccc6)ccc45)nc(-c4cccc5c4oc4ccccc45)n3)cc21] | 83 |
| 1-77 | 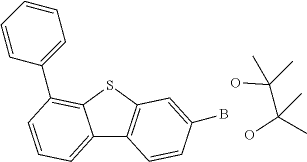 | 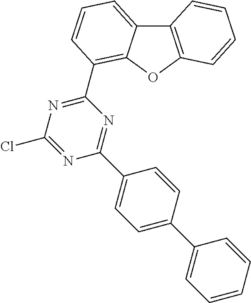 [SMILES: Clc1nc(-c2ccc(-c3ccccc3)cc2)nc(-c2cccc3c2oc2ccccc23)n1] | 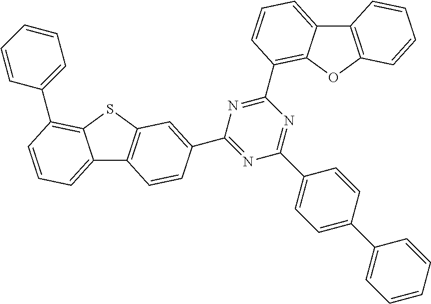 [SMILES: c1ccc(-c2ccc(-c3nc(-c4ccc5c(c4)sc4c(-c6ccccc6)cccc45)nc(-c4cccc5c4oc4ccccc45)n3)cc2)cc1] | 84 |
| 1-78 | 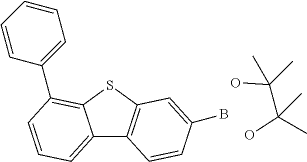 | 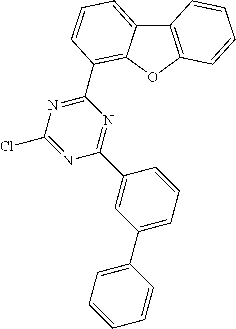 [SMILES: Clc1nc(-c2cccc(-c3ccccc3)c2)nc(-c2cccc3c2oc2ccccc23)n1] | 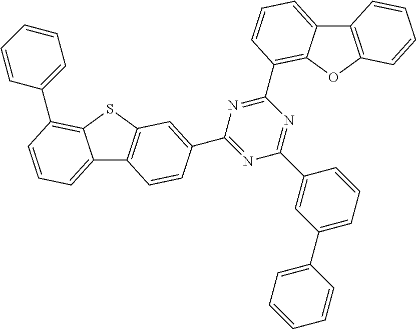 [SMILES: c1ccc(-c2cccc(-c3nc(-c4ccc5c(c4)sc4c(-c6ccccc6)cccc45)nc(-c4cccc5c4oc4ccccc45)n3)c2)cc1] | 83 |
| 1-79 | 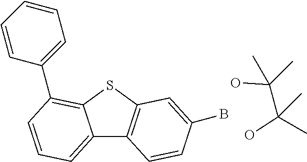 | 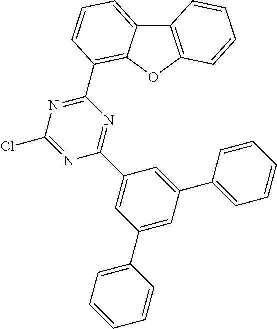 [SMILES: Clc1nc(-c2cc(-c3ccccc3)cc(-c3ccccc3)c2)nc(-c2cccc3c2oc2ccccc23)n1] | 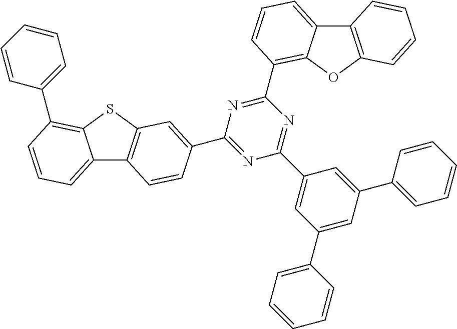 [SMILES: c1ccc(-c2cc(-c3ccccc3)cc(-c3nc(-c4ccc5c(c4)sc4c(-c6ccccc6)cccc45)nc(-c4cccc5c4oc4ccccc45)n3)c2)cc1] | 80 |
| 1-80 | 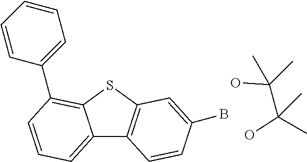 | 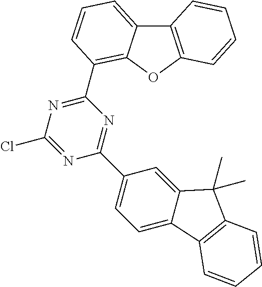 [SMILES: CC1(C)c2ccccc2-c2ccc(-c3nc(Cl)nc(-c4cccc5c4oc4ccccc45)n3)cc21] | 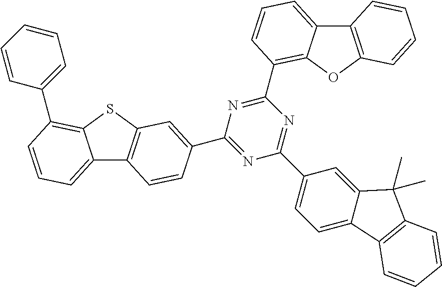 [SMILES: CC1(C)c2ccccc2-c2ccc(-c3nc(-c4ccc5c(c4)sc4c(-c6ccccc6)cccc45)nc(-c4cccc5c4oc4ccccc45)n3)cc21] | 88 |
| 1-101 | 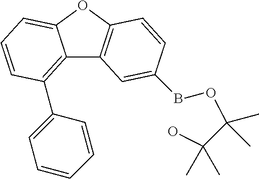 | 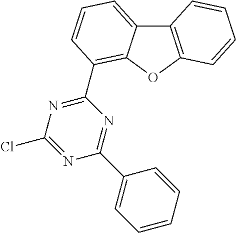 [SMILES: Clc1nc(-c2ccccc2)nc(-c2cccc3c2oc2ccccc23)n1] | 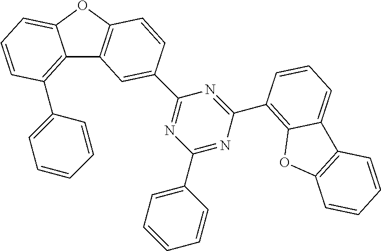 [SMILES: c1ccc(-c2nc(-c3ccc4oc5cccc(-c6ccccc6)c5c4c3)nc(-c3cccc4c3oc3ccccc34)n2)cc1] | 86 |
| 1-102 | 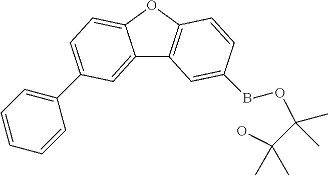 | 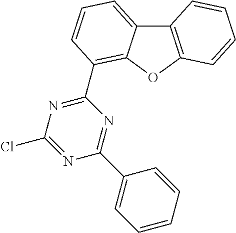 [SMILES: Clc1nc(-c2ccccc2)nc(-c2cccc3c2oc2ccccc23)n1] | 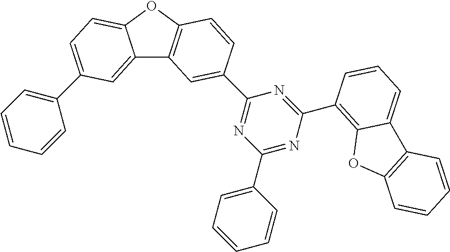 [SMILES: c1ccc(-c2ccc3oc4ccc(-c5nc(-c6ccccc6)nc(-c6cccc7c6oc6ccccc67)n5)cc4c3c2)cc1] | 85 |
| 1-103 | 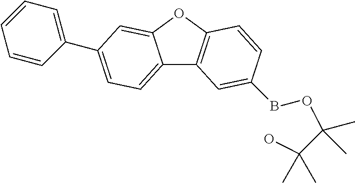 | 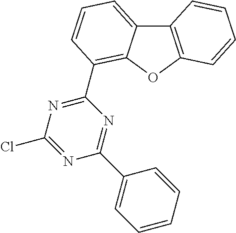 [SMILES: Clc1nc(-c2ccccc2)nc(-c2cccc3c2oc2ccccc23)n1] | 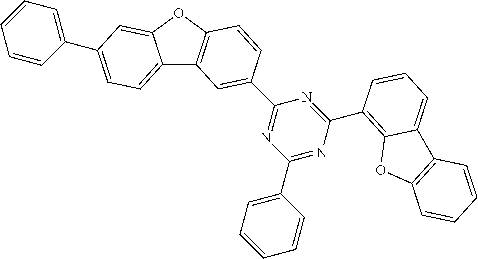 [SMILES: c1ccc(-c2ccc3c(c2)oc2ccc(-c4nc(-c5ccccc5)nc(-c5cccc6c5oc5ccccc56)n4)cc23)cc1] | 83 |
| 1-104 | 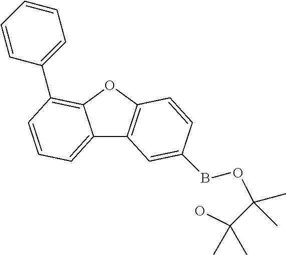 | 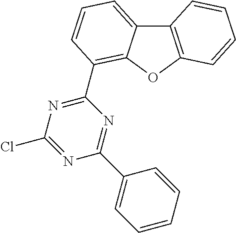 [SMILES: Clc1nc(-c2ccccc2)nc(-c2cccc3c2oc2ccccc23)n1] | 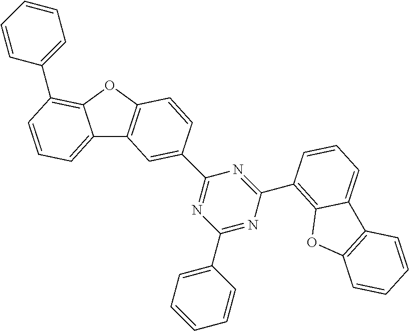 [SMILES: c1ccc(-c2nc(-c3ccc4oc5c(-c6ccccc6)cccc5c4c3)nc(-c3cccc4c3oc3ccccc34)n2)cc1] | 84 |
| 1-105 | 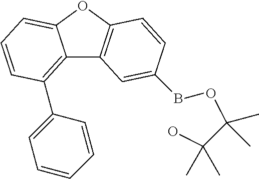 | 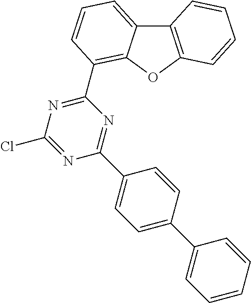 [SMILES: Clc1nc(-c2ccc(-c3ccccc3)cc2)nc(-c2cccc3c2oc2ccccc23)n1] | 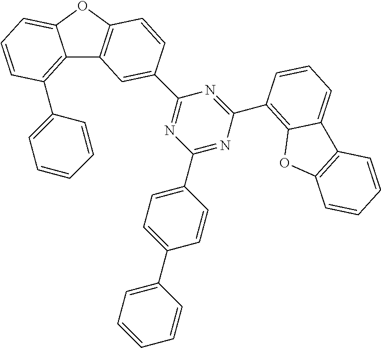 [SMILES: c1ccc(-c2ccc(-c3nc(-c4ccc5oc6cccc(-c7ccccc7)c6c5c4)nc(-c4cccc5c4oc4ccccc45)n3)cc2)cc1] | 88 |
| 1-106 | 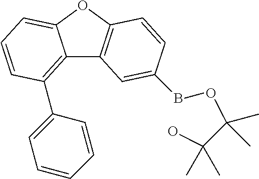 | 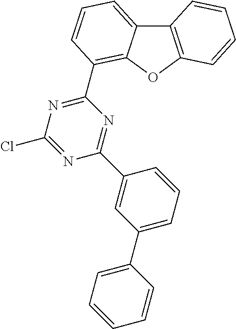 [SMILES: Clc1nc(-c2cccc(-c3ccccc3)c2)nc(-c2cccc3c2oc2ccccc23)n1] | 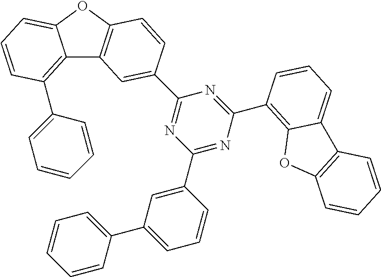 [SMILES: c1ccc(-c2cccc(-c3nc(-c4ccc5oc6cccc(-c7ccccc7)c6c5c4)nc(-c4cccc5c4oc4ccccc45)n3)c2)cc1] | 86 |
| 1-107 | 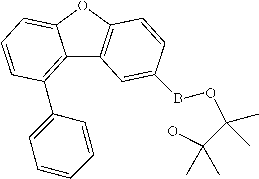 | 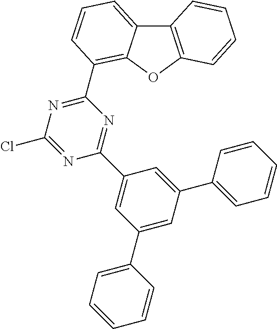 [SMILES: Clc1nc(-c2cc(-c3ccccc3)cc(-c3ccccc3)c2)nc(-c2cccc3c2oc2ccccc23)n1] | 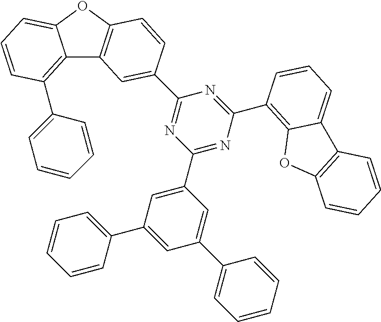 [SMILES: c1ccc(-c2cc(-c3ccccc3)cc(-c3nc(-c4ccc5oc6cccc(-c7ccccc7)c6c5c4)nc(-c4cccc5c4oc4ccccc45)n3)c2)cc1] | 84 |
| 1-108 | 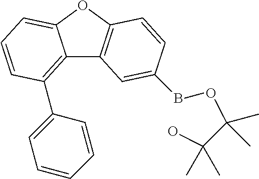 | 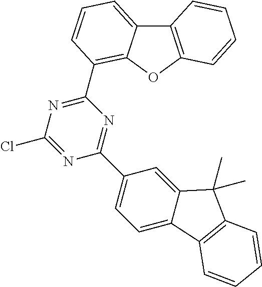 [SMILES: CC1(C)c2ccccc2-c2ccc(-c3nc(Cl)nc(-c4cccc5c4oc4ccccc45)n3)cc21] | 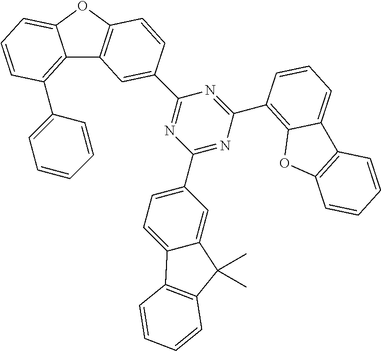 [SMILES: CC1(C)c2ccccc2-c2ccc(-c3nc(-c4ccc5oc6cccc(-c7ccccc7)c6c5c4)nc(-c4cccc5c4oc4ccccc45)n3)cc21] | 85 |
| 1-109 | 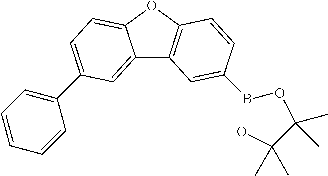 | 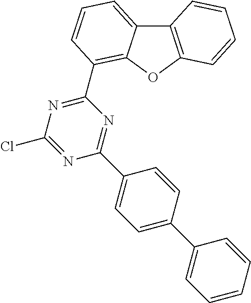 [SMILES: Clc1nc(-c2ccc(-c3ccccc3)cc2)nc(-c2cccc3c2oc2ccccc23)n1] | 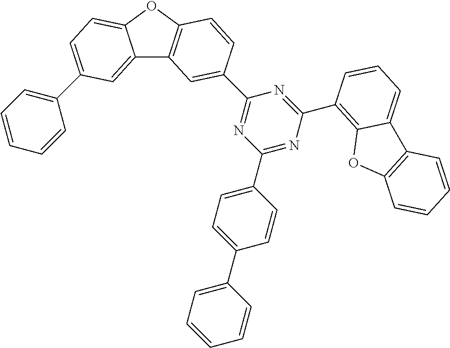 [SMILES: c1ccc(-c2ccc(-c3nc(-c4ccc5oc6ccc(-c7ccccc7)cc6c5c4)nc(-c4cccc5c4oc4ccccc45)n3)cc2)cc1] | 83 |
| 1-110 | 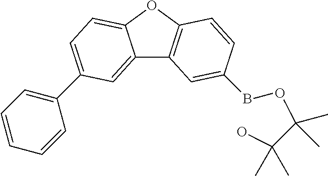 | 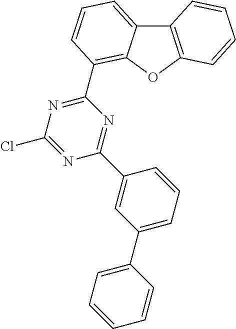 [SMILES: Clc1nc(-c2cccc(-c3ccccc3)c2)nc(-c2cccc3c2oc2ccccc23)n1] | 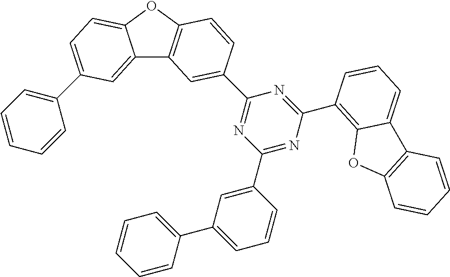 [SMILES: c1ccc(-c2cccc(-c3nc(-c4ccc5oc6ccc(-c7ccccc7)cc6c5c4)nc(-c4cccc5c4oc4ccccc45)n3)c2)cc1] | 85 |
| 1-111 | 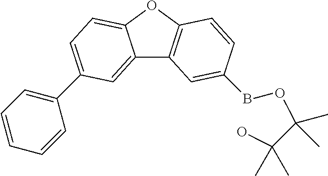 | 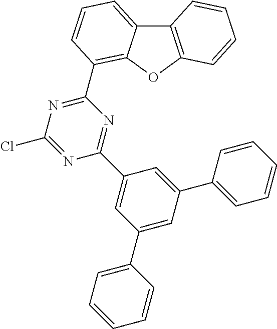 [SMILES: Clc1nc(-c2cc(-c3ccccc3)cc(-c3ccccc3)c2)nc(-c2cccc3c2oc2ccccc23)n1] | 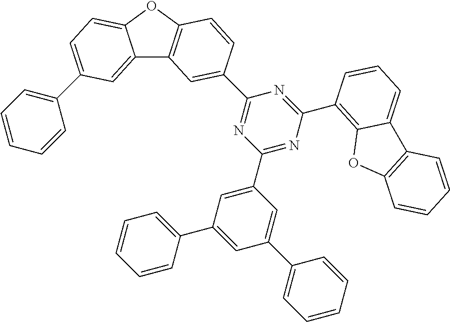 [SMILES: c1ccc(-c2cc(-c3ccccc3)cc(-c3nc(-c4ccc5oc6ccc(-c7ccccc7)cc6c5c4)nc(-c4cccc5c4oc4ccccc45)n3)c2)cc1] | 83 |
| 1-112 | 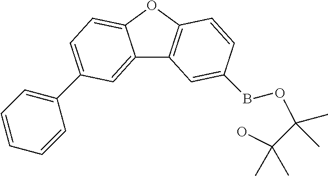 | 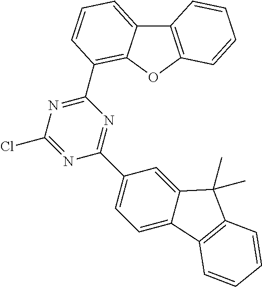 [SMILES: CC1(C)c2ccccc2-c2ccc(-c3nc(Cl)nc(-c4cccc5c4oc4ccccc45)n3)cc21] | 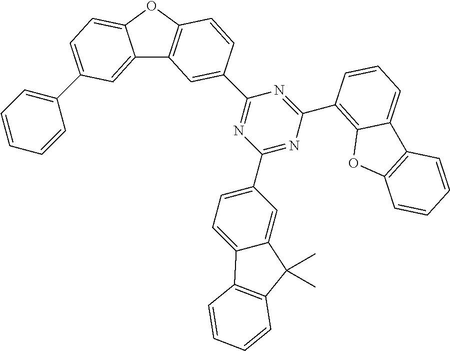 [SMILES: CC1(C)c2ccccc2-c2ccc(-c3nc(-c4ccc5oc6ccc(-c7ccccc7)cc6c5c4)nc(-c4cccc5c4oc4ccccc45)n3)cc21] | 80 |
| 1-113 | 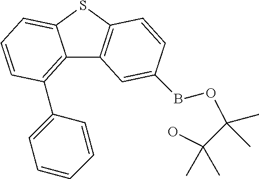 | 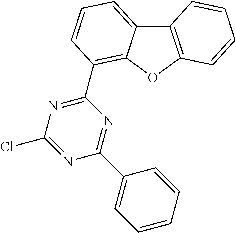 [SMILES: Clc1nc(-c2ccccc2)nc(-c2cccc3c2oc2ccccc23)n1] | 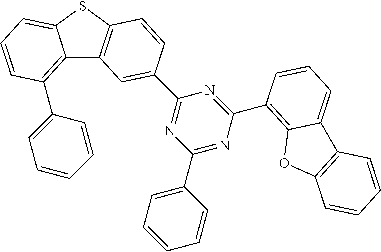 [SMILES: c1ccc(-c2nc(-c3ccc4sc5cccc(-c6ccccc6)c5c4c3)nc(-c3cccc4c3oc3ccccc34)n2)cc1] | 81 |
| 1-114 | 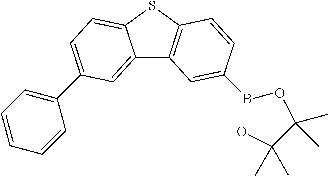 | 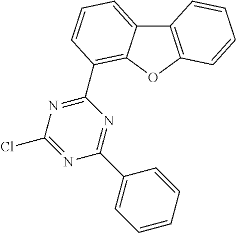 [SMILES: Clc1nc(-c2ccccc2)nc(-c2cccc3c2oc2ccccc23)n1] | 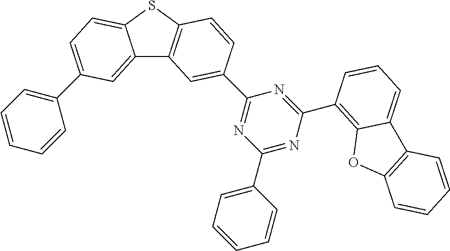 [SMILES: c1ccc(-c2ccc3sc4ccc(-c5nc(-c6ccccc6)nc(-c6cccc7c6oc6ccccc67)n5)cc4c3c2)cc1] | 82 |
| 1-115 | 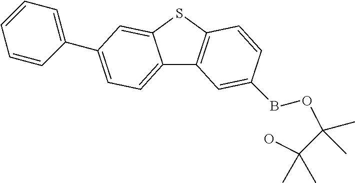 | 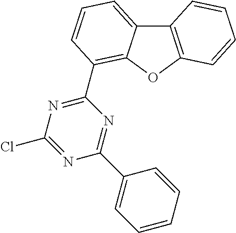 [SMILES: Clc1nc(-c2ccccc2)nc(-c2cccc3c2oc2ccccc23)n1] | 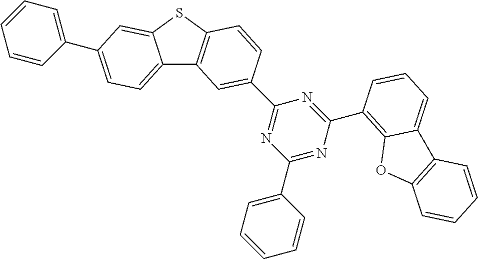 [SMILES: c1ccc(-c2ccc3c(c2)sc2ccc(-c4nc(-c5ccccc5)nc(-c5cccc6c5oc5ccccc56)n4)cc23)cc1] | 83 |
| 1-116 | 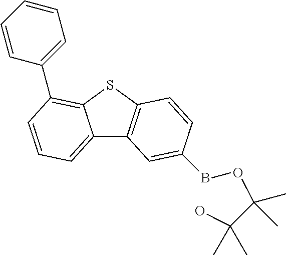 | 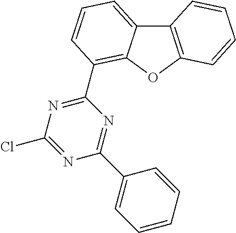 [SMILES: Clc1nc(-c2ccccc2)nc(-c2cccc3c2oc2ccccc23)n1] | 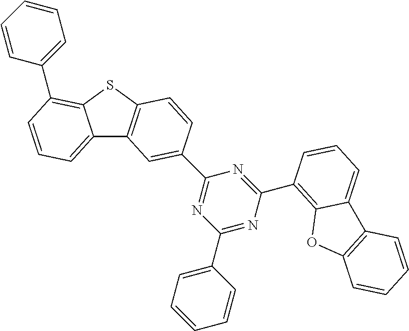 [SMILES: c1ccc(-c2nc(-c3ccc4sc5c(-c6ccccc6)cccc5c4c3)nc(-c3cccc4c3oc3ccccc34)n2)cc1] | 88 |
| 1-117 | 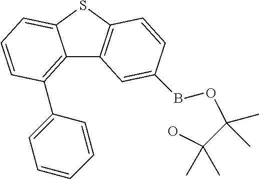 | 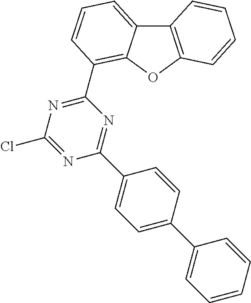 [SMILES: Clc1nc(-c2ccc(-c3ccccc3)cc2)nc(-c2cccc3c2oc2ccccc23)n1] | 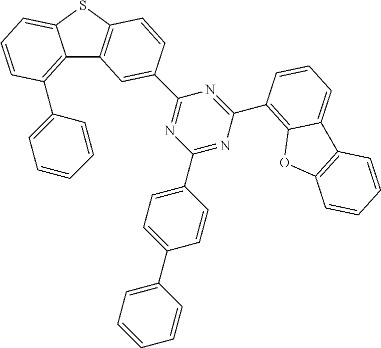 [SMILES: c1ccc(-c2ccc(-c3nc(-c4ccc5sc6cccc(-c7ccccc7)c6c5c4)nc(-c4cccc5c4oc4ccccc45)n3)cc2)cc1] | 85 |
| 1-118 | 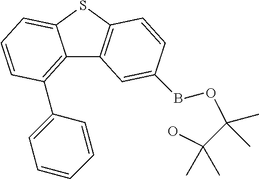 | 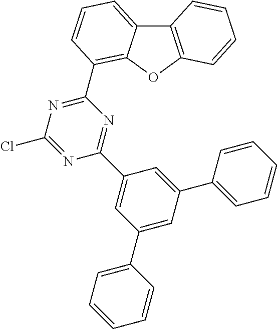 [SMILES: Clc1nc(-c2cc(-c3ccccc3)cc(-c3ccccc3)c2)nc(-c2cccc3c2oc2ccccc23)n1] | 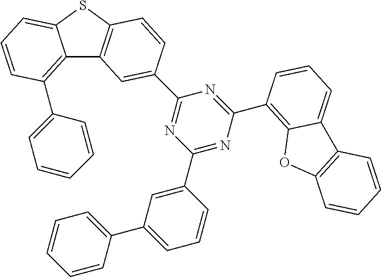 [SMILES: c1ccc(-c2cccc(-c3nc(-c4ccc5sc6cccc(-c7ccccc7)c6c5c4)nc(-c4cccc5c4oc4ccccc45)n3)c2)cc1] | 83 |
| 1-119 | 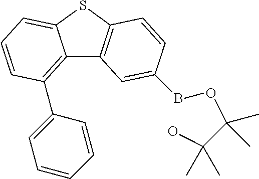 | 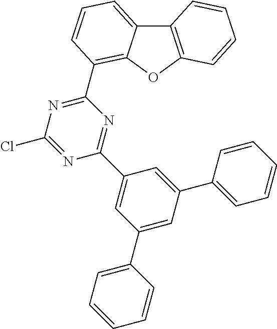 [SMILES: Clc1nc(-c2cc(-c3ccccc3)cc(-c3ccccc3)c2)nc(-c2cccc3c2oc2ccccc23)n1] | 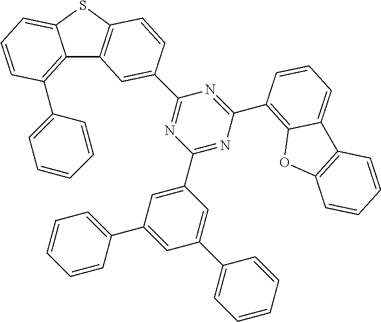 [SMILES: c1ccc(-c2cc(-c3ccccc3)cc(-c3nc(-c4ccc5sc6cccc(-c7ccccc7)c6c5c4)nc(-c4cccc5c4oc4ccccc45)n3)c2)cc1] | 84 |
| 1-120 | 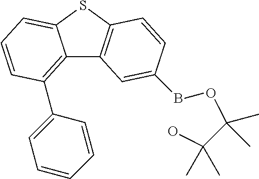 | 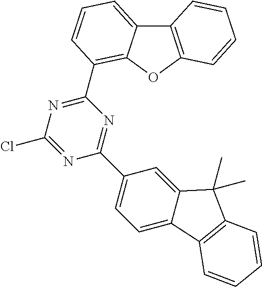 [SMILES: CC1(C)c2ccccc2-c2ccc(-c3nc(Cl)nc(-c4cccc5c4oc4ccccc45)n3)cc21] | 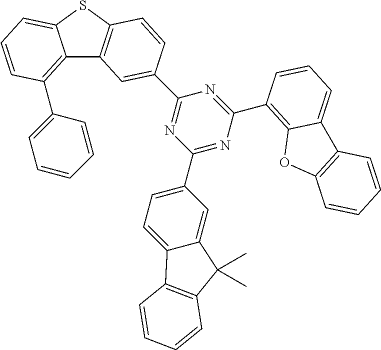 [SMILES: CC1(C)c2ccccc2-c2ccc(-c3nc(-c4ccc5sc6cccc(-c7ccccc7)c6c5c4)nc(-c4cccc5c4oc4ccccc45)n3)cc21] | 83 |
| 1-121 | 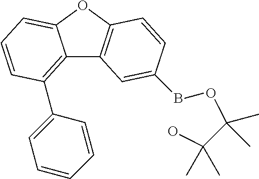 | 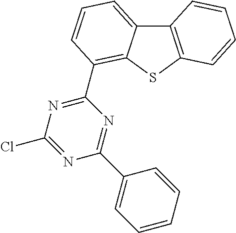 [SMILES: Clc1nc(-c2ccccc2)nc(-c2cccc3c2sc2ccccc23)n1] | 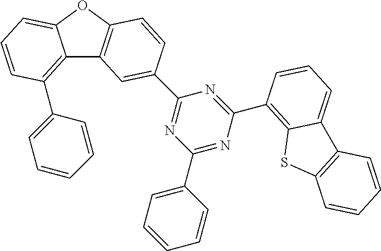 [SMILES: c1ccc(-c2nc(-c3ccc4oc5cccc(-c6ccccc6)c5c4c3)nc(-c3cccc4c3sc3ccccc34)n2)cc1] | 80 |
| 1-122 | 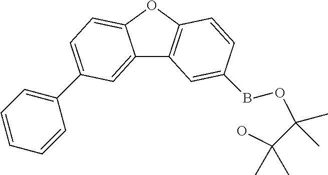 | 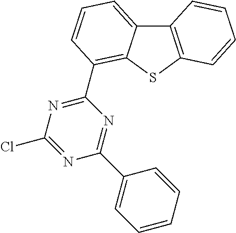 [SMILES: Clc1nc(-c2ccccc2)nc(-c2cccc3c2sc2ccccc23)n1] | 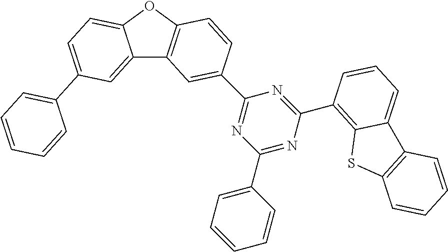 [SMILES: c1ccc(-c2ccc3oc4ccc(-c5nc(-c6ccccc6)nc(-c6cccc7c6sc6ccccc67)n5)cc4c3c2)cc1] | 88 |
| 1-123 | 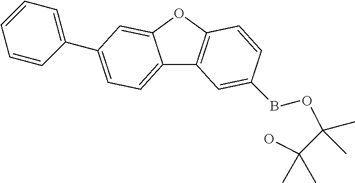 | 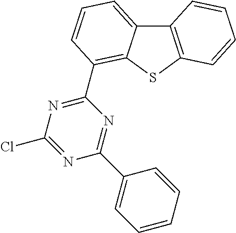 [SMILES: Clc1nc(-c2ccccc2)nc(-c2cccc3c2sc2ccccc23)n1] | 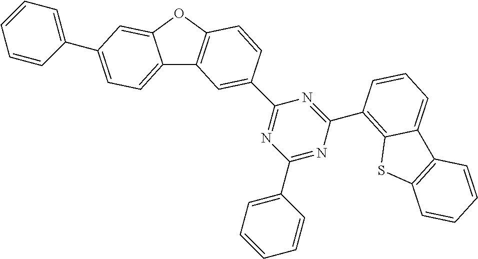 [SMILES: c1ccc(-c2ccc3c(c2)oc2ccc(-c4nc(-c5ccccc5)nc(-c5cccc6c5sc5ccccc56)n4)cc23)cc1] | 86 |
| 1-124 | 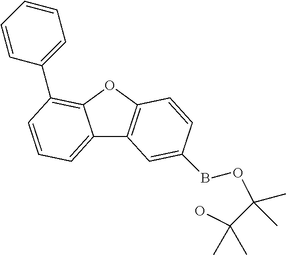 | 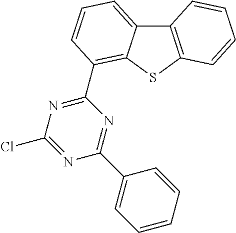 [SMILES: Clc1nc(-c2ccccc2)nc(-c2cccc3c2sc2ccccc23)n1] | 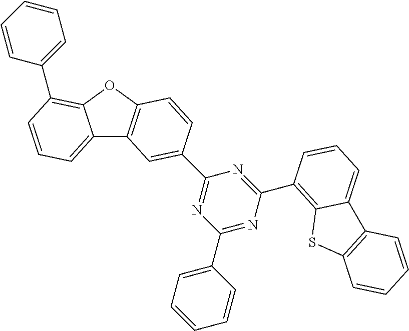 [SMILES: c1ccc(-c2nc(-c3ccc4oc5c(-c6ccccc6)cccc5c4c3)nc(-c3cccc4c3sc3ccccc34)n2)cc1] | 85 |
| 1-125 | 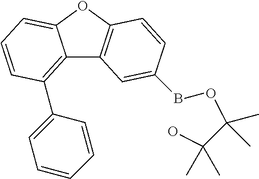 | 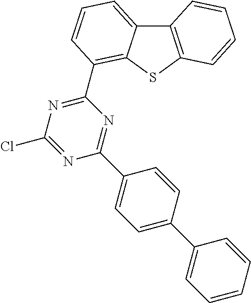 [SMILES: Clc1nc(-c2ccc(-c3ccccc3)cc2)nc(-c2cccc3c2sc2ccccc23)n1] | 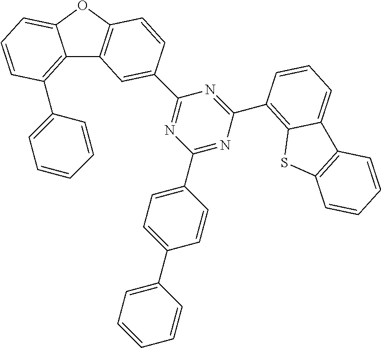 [SMILES: c1ccc(-c2ccc(-c3nc(-c4ccc5oc6cccc(-c7ccccc7)c6c5c4)nc(-c4cccc5c4sc4ccccc45)n3)cc2)cc1] | 83 |
| 1-126 | 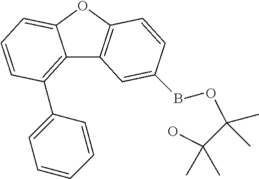 | 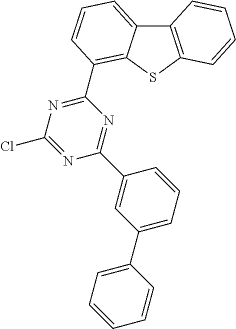 [SMILES: Clc1nc(-c2cccc(-c3ccccc3)c2)nc(-c2cccc3c2sc2ccccc23)n1] | 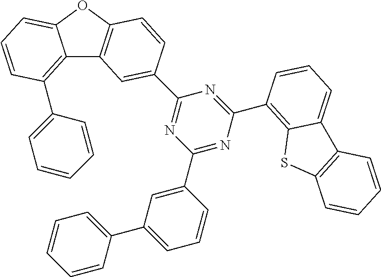 [SMILES: c1ccc(-c2cccc(-c3nc(-c4ccc5oc6cccc(-c7ccccc7)c6c5c4)nc(-c4cccc5c4sc4ccccc45)n3)c2)cc1] | 84 |
| 1-127 | 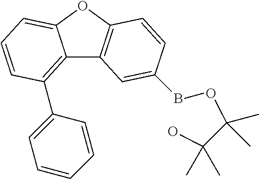 | 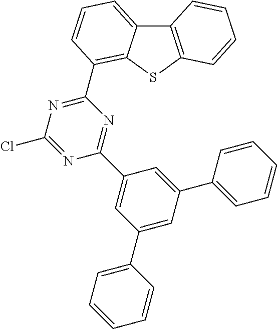 [SMILES: Clc1nc(-c2cc(-c3ccccc3)cc(-c3ccccc3)c2)nc(-c2cccc3c2sc2ccccc23)n1] | 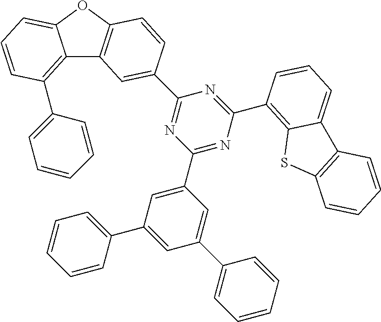 [SMILES: c1ccc(-c2cc(-c3ccccc3)cc(-c3nc(-c4ccc5oc6cccc(-c7ccccc7)c6c5c4)nc(-c4cccc5c4sc4ccccc45)n3)c2)cc1] | 88 |
| 1-128 | 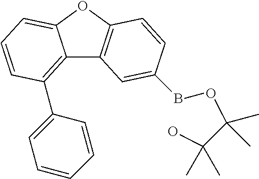 | 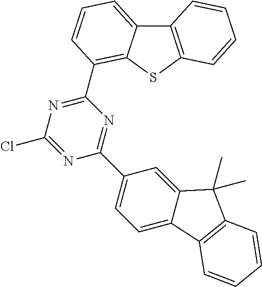 [SMILES: CC1(C)c2ccccc2-c2ccc(-c3nc(Cl)nc(-c4cccc5c4sc4ccccc45)n3)cc21] | 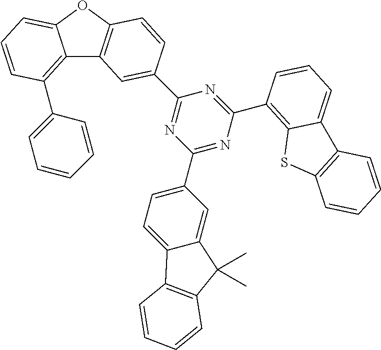 [SMILES: CC1(C)c2ccccc2-c2ccc(-c3nc(-c4ccc5oc6cccc(-c7ccccc7)c6c5c4)nc(-c4cccc5c4sc4ccccc45)n3)cc21] | 86 |
| 1-129 | 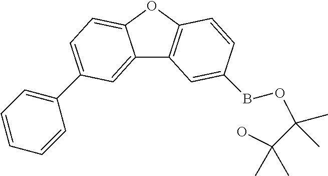 | 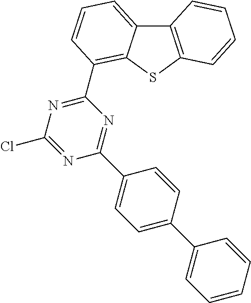 [SMILES: Clc1nc(-c2ccc(-c3ccccc3)cc2)nc(-c2cccc3c2sc2ccccc23)n1] | 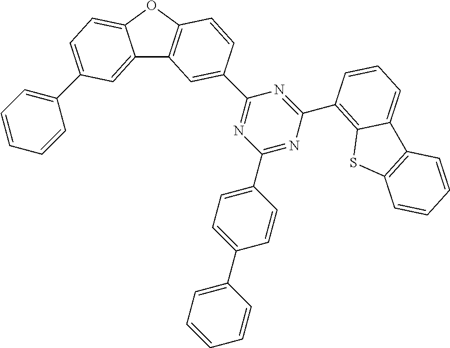 [SMILES: c1ccc(-c2ccc(-c3nc(-c4ccc5oc6ccc(-c7ccccc7)cc6c5c4)nc(-c4cccc5c4sc4ccccc45)n3)cc2)cc1] | 84 |
| 1-130 | 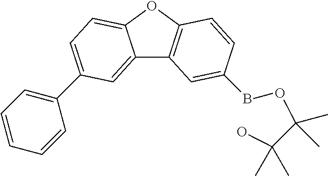 | 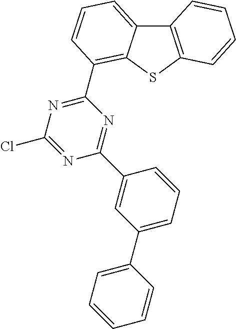 [SMILES: Clc1nc(-c2cccc(-c3ccccc3)c2)nc(-c2cccc3c2sc2ccccc23)n1] | 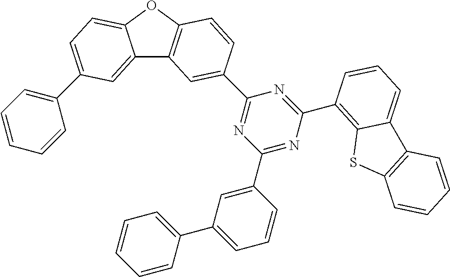 [SMILES: c1ccc(-c2cccc(-c3nc(-c4ccc5oc6ccc(-c7ccccc7)cc6c5c4)nc(-c4cccc5c4sc4ccccc45)n3)c2)cc1] | 85 |
| 1-131 | 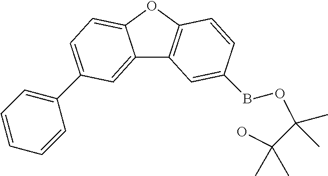 | 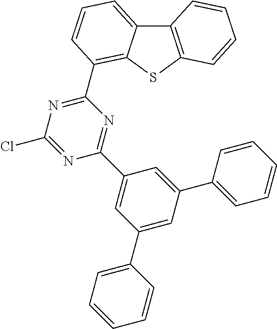 [SMILES: Clc1nc(-c2cc(-c3ccccc3)cc(-c3ccccc3)c2)nc(-c2cccc3c2sc2ccccc23)n1] | 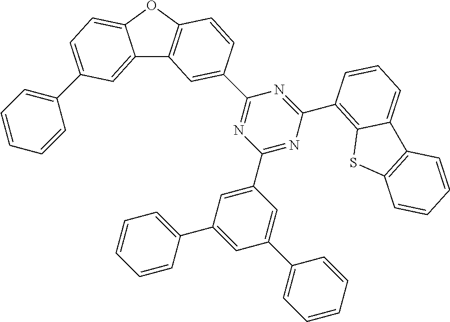 [SMILES: c1ccc(-c2cc(-c3ccccc3)cc(-c3nc(-c4ccc5oc6ccc(-c7ccccc7)cc6c5c4)nc(-c4cccc5c4sc4ccccc45)n3)c2)cc1] | 83 |
| 1-132 | 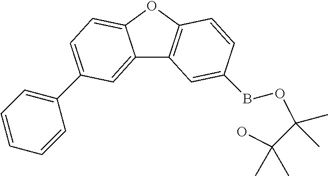 | 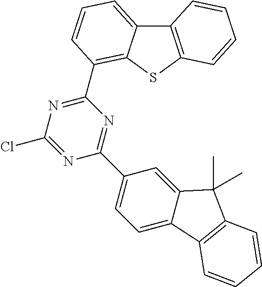 [SMILES: CC1(C)c2ccccc2-c2ccc(-c3nc(Cl)nc(-c4cccc5c4sc4ccccc45)n3)cc21] | 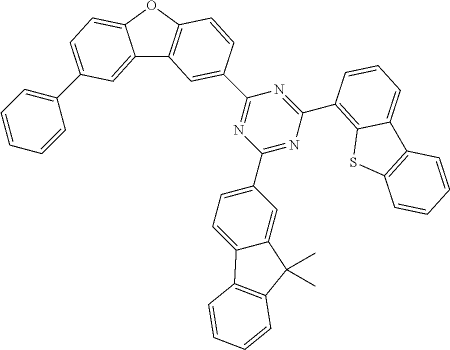 [SMILES: CC1(C)c2ccccc2-c2ccc(-c3nc(-c4ccc5oc6ccc(-c7ccccc7)cc6c5c4)nc(-c4cccc5c4sc4ccccc45)n3)cc21] | 85 |
| 1-133 | 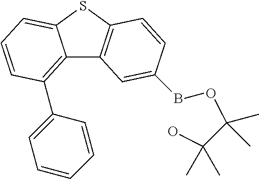 | 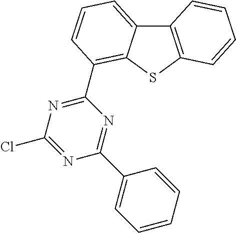 [SMILES: Clc1nc(-c2ccccc2)nc(-c2cccc3c2sc2ccccc23)n1] | 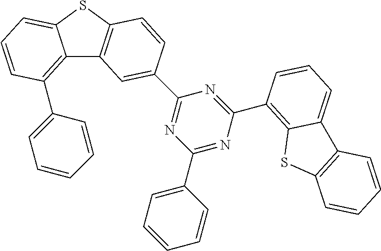 [SMILES: c1ccc(-c2nc(-c3ccc4sc5cccc(-c6ccccc6)c5c4c3)nc(-c3cccc4c3sc3ccccc34)n2)cc1] | 83 |
| 1-134 | 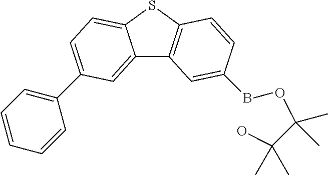 | 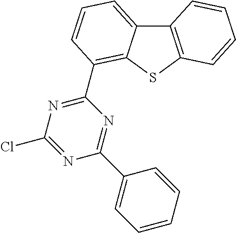 [SMILES: Clc1nc(-c2ccccc2)nc(-c2cccc3c2sc2ccccc23)n1] | 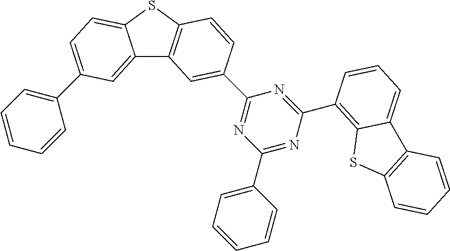 [SMILES: c1ccc(-c2ccc3sc4ccc(-c5nc(-c6ccccc6)nc(-c6cccc7c6sc6ccccc67)n5)cc4c3c2)cc1] | 80 |
| 1-135 | 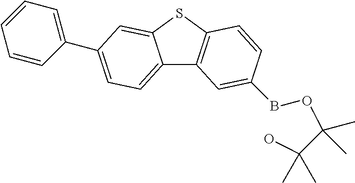 | 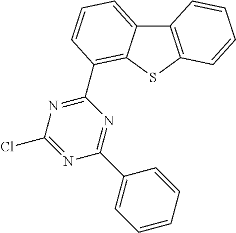 [SMILES: Clc1nc(-c2ccccc2)nc(-c2cccc3c2sc2ccccc23)n1] | 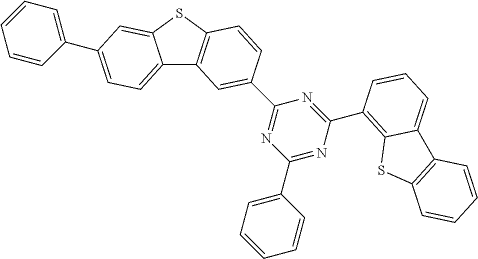 [SMILES: c1ccc(-c2ccc3c(c2)sc2ccc(-c4nc(-c5ccccc5)nc(-c5cccc6c5sc5ccccc56)n4)cc23)cc1] | 81 |
| 1-136 | 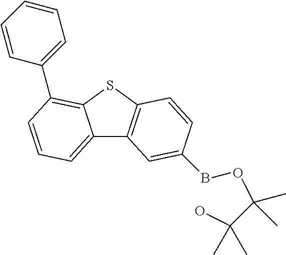 | 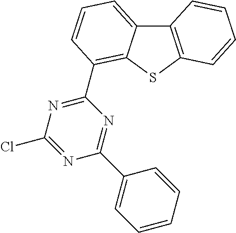 [SMILES: Clc1nc(-c2ccccc2)nc(-c2cccc3c2sc2ccccc23)n1] | 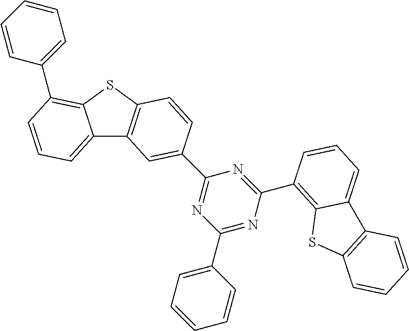 [SMILES: c1ccc(-c2nc(-c3ccc4sc5c(-c6ccccc6)cccc5c4c3)nc(-c3cccc4c3sc3ccccc34)n2)cc1] | 82 |
| 1-137 | 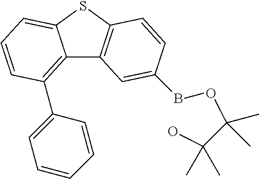 | 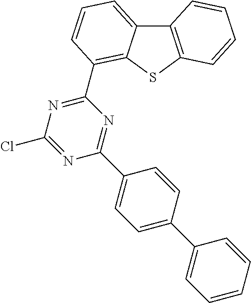 [SMILES: Clc1nc(-c2ccc(-c3ccccc3)cc2)nc(-c2cccc3c2sc2ccccc23)n1] | 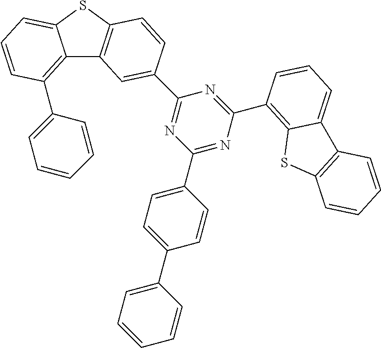 [SMILES: c1ccc(-c2ccc(-c3nc(-c4ccc5sc6cccc(-c7ccccc7)c6c5c4)nc(-c4cccc5c4sc4ccccc45)n3)cc2)cc1] | 83 |
| 1-138 | 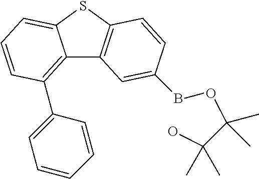 | 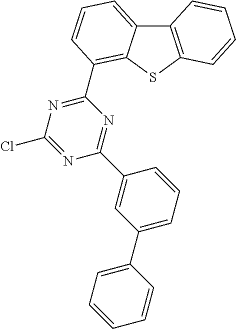 [SMILES: Clc1nc(-c2cccc(-c3ccccc3)c2)nc(-c2cccc3c2sc2ccccc23)n1] | 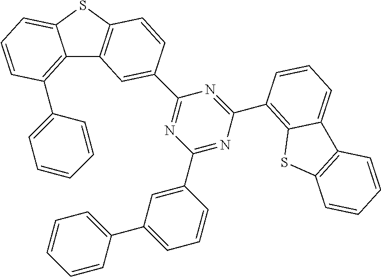 [SMILES: c1ccc(-c2cccc(-c3nc(-c4ccc5sc6cccc(-c7ccccc7)c6c5c4)nc(-c4cccc5c4sc4ccccc45)n3)c2)cc1] | 88 |
| 1-139 | 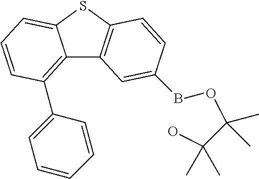 | 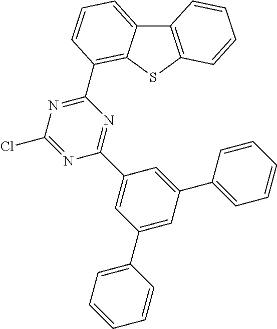 [SMILES: Clc1nc(-c2cc(-c3ccccc3)cc(-c3ccccc3)c2)nc(-c2cccc3c2sc2ccccc23)n1] | 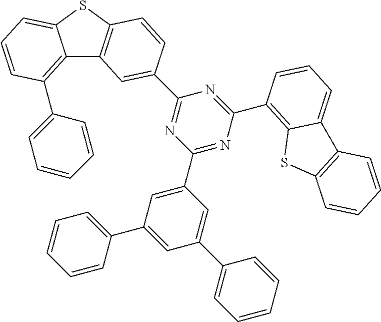 [SMILES: c1ccc(-c2cc(-c3ccccc3)cc(-c3nc(-c4ccc5sc6cccc(-c7ccccc7)c6c5c4)nc(-c4cccc5c4sc4ccccc45)n3)c2)cc1] | 85 |
| 1-140 | 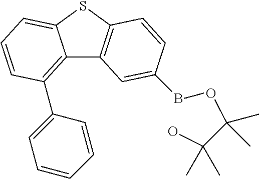 | 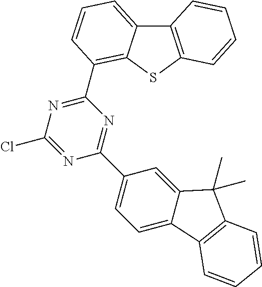 [SMILES: CC1(C)c2ccccc2-c2ccc(-c3nc(Cl)nc(-c4cccc5c4sc4ccccc45)n3)cc21] | 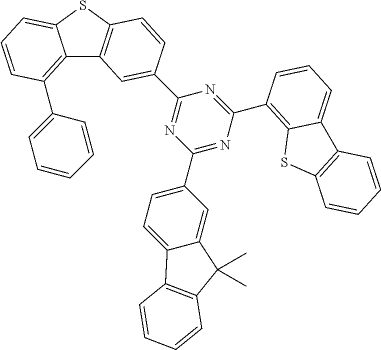 [SMILES: CC1(C)c2ccccc2-c2ccc(-c3nc(-c4ccc5sc6cccc(-c7ccccc7)c6c5c4)nc(-c4cccc5c4sc4ccccc45)n3)cc21] | 83 |
| 1-141 | 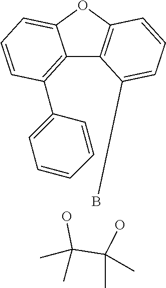 | 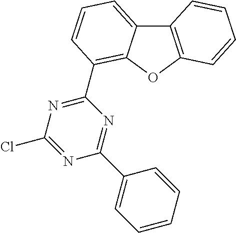 [SMILES: Clc1nc(-c2ccccc2)nc(-c2cccc3c2oc2ccccc23)n1] | 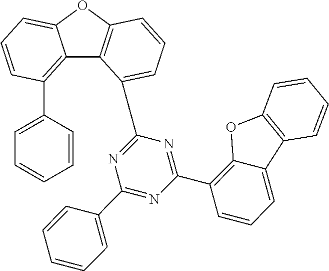 [SMILES: c1ccc(-c2nc(-c3cccc4c3oc3ccccc34)nc(-c3cccc4oc5cccc(-c6ccccc6)c5c34)n2)cc1] | 84 |
| 1-142 | 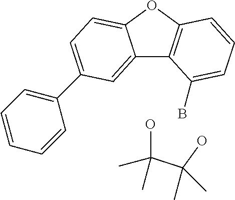 | 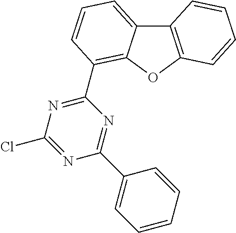 [SMILES: Clc1nc(-c2ccccc2)nc(-c2cccc3c2oc2ccccc23)n1] | 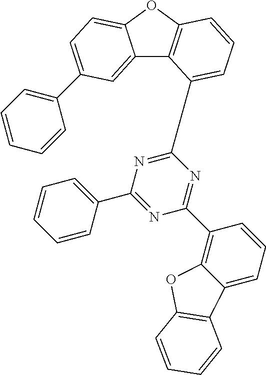 [SMILES: c1ccc(-c2ccc3oc4cccc(-c5nc(-c6ccccc6)nc(-c6cccc7c6oc6ccccc67)n5)c4c3c2)cc1] | 83 |
| 1-143 | 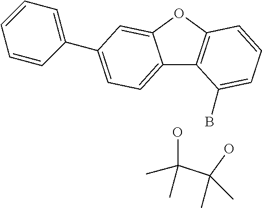 | 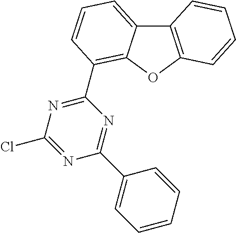 [SMILES: Clc1nc(-c2ccccc2)nc(-c2cccc3c2oc2ccccc23)n1] | 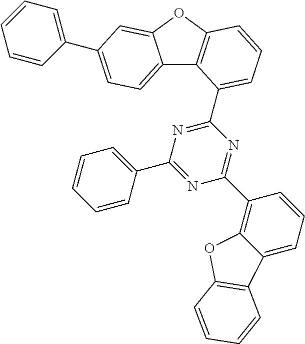 [SMILES: c1ccc(-c2ccc3c(c2)oc2cccc(-c4nc(-c5ccccc5)nc(-c5cccc6c5oc5ccccc56)n4)c23)cc1] | 80 |
| 1-144 | 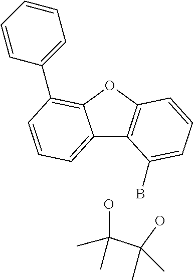 | 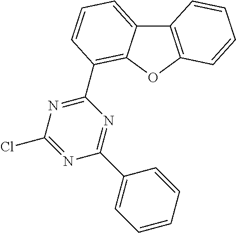 [SMILES: Clc1nc(-c2ccccc2)nc(-c2cccc3c2oc2ccccc23)n1] | 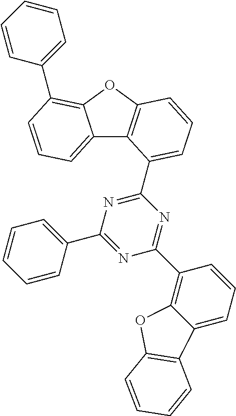 [SMILES: c1ccc(-c2nc(-c3cccc4c3oc3ccccc34)nc(-c3cccc4oc5c(-c6ccccc6)cccc5c34)n2)cc1] | 88 |
| 1-145 | 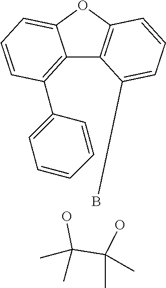 | 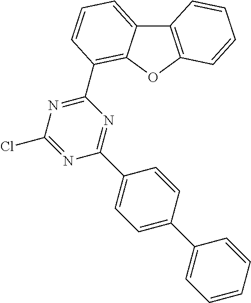 [SMILES: Clc1nc(-c2ccc(-c3ccccc3)cc2)nc(-c2cccc3c2oc2ccccc23)n1] | 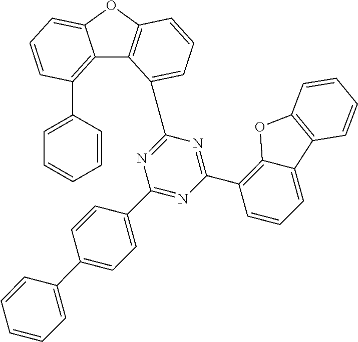 [SMILES: c1ccc(-c2ccc(-c3nc(-c4cccc5c4oc4ccccc45)nc(-c4cccc5oc6cccc(-c7ccccc7)c6c45)n3)cc2)cc1] | 86 |
| 1-146 | 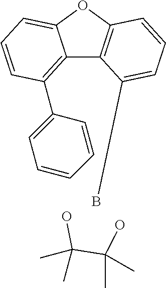 | 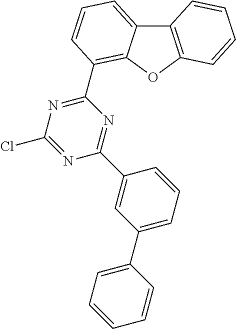 [SMILES: Clc1nc(-c2cccc(-c3ccccc3)c2)nc(-c2cccc3c2oc2ccccc23)n1] | 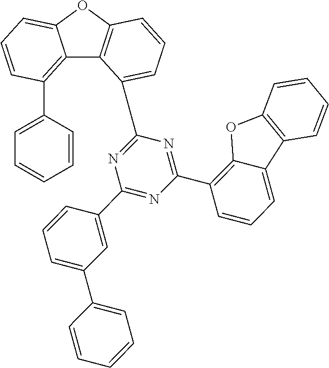 [SMILES: c1ccc(-c2cccc(-c3nc(-c4cccc5c4oc4ccccc45)nc(-c4cccc5oc6cccc(-c7ccccc7)c6c45)n3)c2)cc1] | 85 |
| 1-147 | 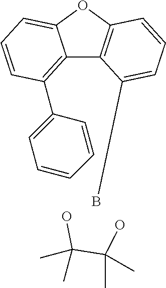 | 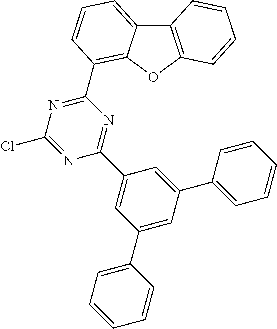 [SMILES: Clc1nc(-c2cc(-c3ccccc3)cc(-c3ccccc3)c2)nc(-c2cccc3c2oc2ccccc23)n1] | 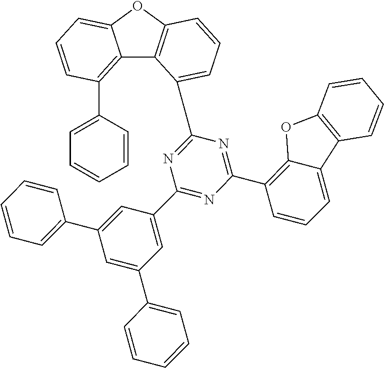 [SMILES: c1ccc(-c2cc(-c3ccccc3)cc(-c3nc(-c4cccc5c4oc4ccccc45)nc(-c4cccc5oc6cccc(-c7ccccc7)c6c45)n3)c2)cc1] | 83 |
| 1-148 | 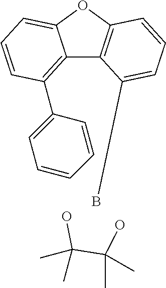 | 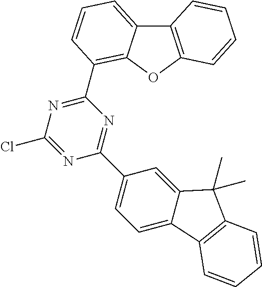 [SMILES: CC1(C)c2ccccc2-c2ccc(-c3nc(Cl)nc(-c4cccc5c4oc4ccccc45)n3)cc21] | 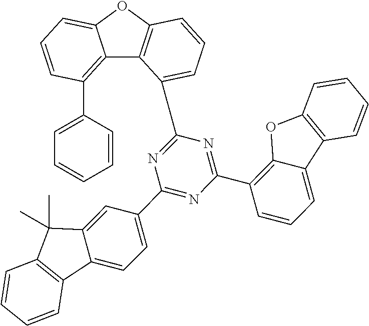 [SMILES: CC1(C)c2ccccc2-c2ccc(-c3nc(-c4cccc5c4oc4ccccc45)nc(-c4cccc5oc6cccc(-c7ccccc7)c6c45)n3)cc21] | 84 |
| 1-149 | 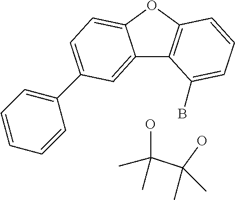 | 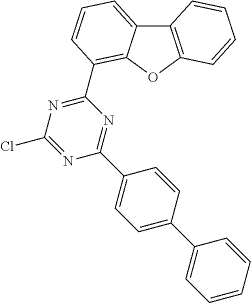 [SMILES: Clc1nc(-c2ccc(-c3ccccc3)cc2)nc(-c2cccc3c2oc2ccccc23)n1] | 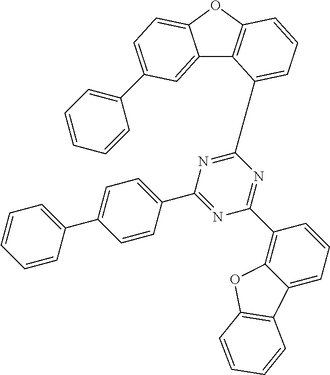 [SMILES: c1ccc(-c2ccc(-c3nc(-c4cccc5c4oc4ccccc45)nc(-c4cccc5oc6ccc(-c7ccccc7)cc6c45)n3)cc2)cc1] | 88 |
| 1-150 | 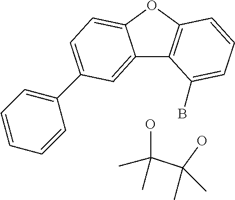 | 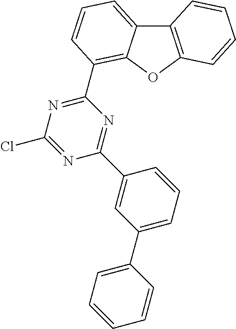 [SMILES: Clc1nc(-c2cccc(-c3ccccc3)c2)nc(-c2cccc3c2oc2ccccc23)n1] | 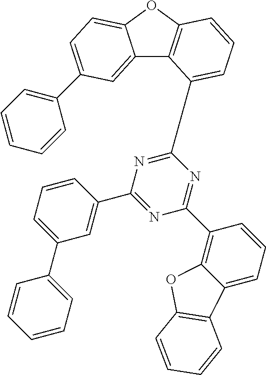 [SMILES: c1ccc(-c2cccc(-c3nc(-c4cccc5c4oc4ccccc45)nc(-c4cccc5oc6ccc(-c7ccccc7)cc6c45)n3)c2)cc1] | 86 |
| 1-151 | 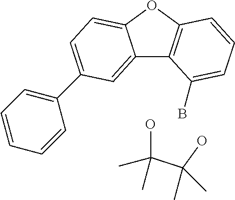 | 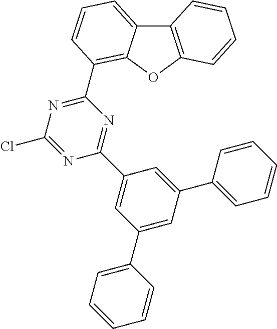 [SMILES: Clc1nc(-c2cc(-c3ccccc3)cc(-c3ccccc3)c2)nc(-c2cccc3c2oc2ccccc23)n1] | 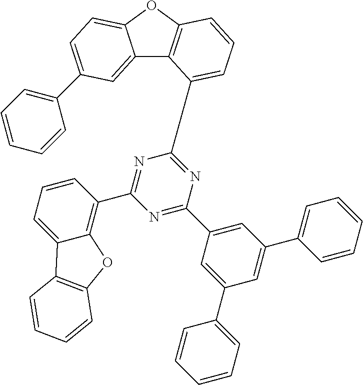 [SMILES: c1ccc(-c2cc(-c3ccccc3)cc(-c3nc(-c4cccc5c4oc4ccccc45)nc(-c4cccc5oc6ccc(-c7ccccc7)cc6c45)n3)c2)cc1] | 84 |
| 1-152 | 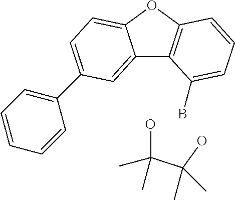 | 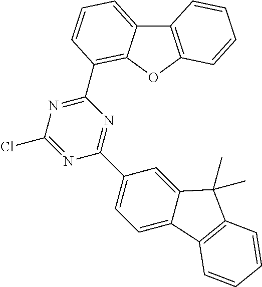 [SMILES: CC1(C)c2ccccc2-c2ccc(-c3nc(Cl)nc(-c4cccc5c4oc4ccccc45)n3)cc21] | 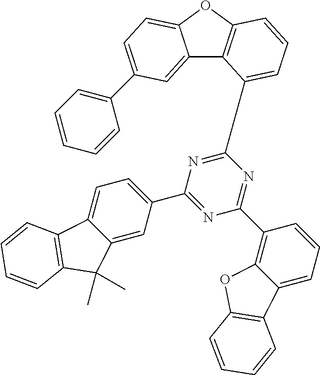 [SMILES: CC1(C)c2ccccc2-c2ccc(-c3nc(-c4cccc5c4oc4ccccc45)nc(-c4cccc5oc6ccc(-c7ccccc7)cc6c45)n3)cc21] | 85 |
| 1-153 | 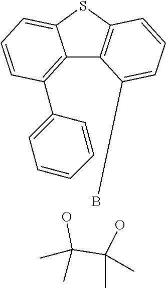 | 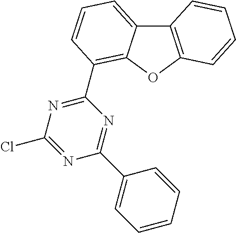 [SMILES: Clc1nc(-c2ccccc2)nc(-c2cccc3c2oc2ccccc23)n1] | 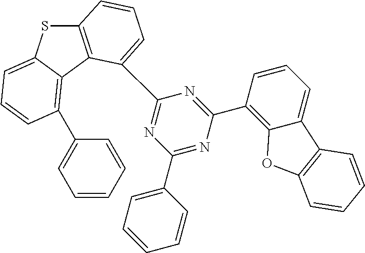 [SMILES: c1ccc(-c2nc(-c3cccc4c3oc3ccccc34)nc(-c3cccc4sc5cccc(-c6ccccc6)c5c34)n2)cc1] | 83 |
| 1-154 | 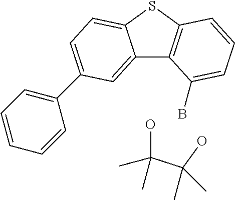 | 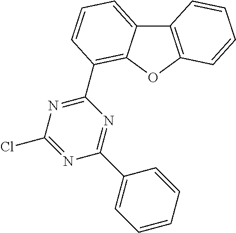 [SMILES: Clc1nc(-c2ccccc2)nc(-c2cccc3c2oc2ccccc23)n1] | 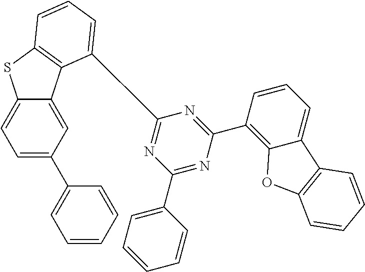 [SMILES: c1ccc(-c2ccc3sc4cccc(-c5nc(-c6ccccc6)nc(-c6cccc7c6oc6ccccc67)n5)c4c3c2)cc1] | 85 |
| 1-155 | 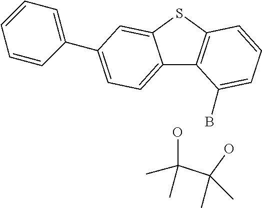 | 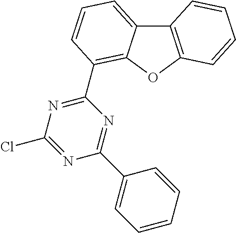 [SMILES: Clc1nc(-c2ccccc2)nc(-c2cccc3c2oc2ccccc23)n1] | 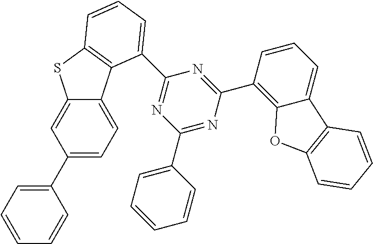 [SMILES: c1ccc(-c2ccc3c(c2)sc2cccc(-c4nc(-c5ccccc5)nc(-c5cccc6c5oc5ccccc56)n4)c23)cc1] | 83 |
| 1-156 | 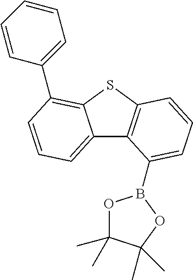 [SMILES: CC1(C)OB(c2cccc3sc4c(-c5ccccc5)cccc4c23)OC1(C)C] | 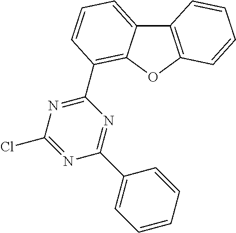 [SMILES: Clc1nc(-c2ccccc2)nc(-c2cccc3c2oc2ccccc23)n1] | 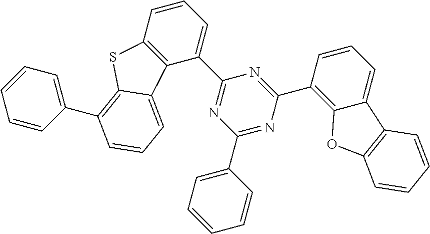 [SMILES: c1ccc(-c2nc(-c3cccc4c3oc3ccccc34)nc(-c3cccc4sc5c(-c6ccccc6)cccc5c34)n2)cc1] | 80 |
| 1-157 | 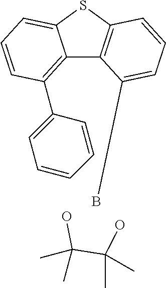 | 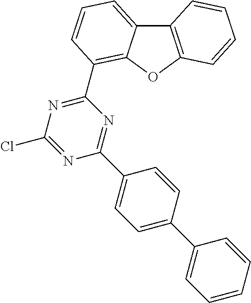 [SMILES: Clc1nc(-c2ccc(-c3ccccc3)cc2)nc(-c2cccc3c2oc2ccccc23)n1] | 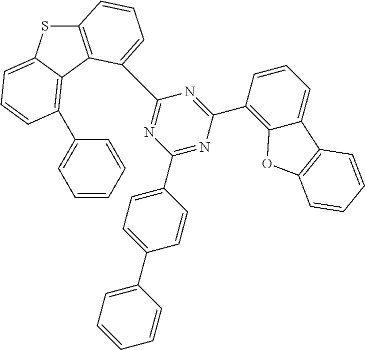 [SMILES: c1ccc(-c2ccc(-c3nc(-c4cccc5c4oc4ccccc45)nc(-c4cccc5sc6cccc(-c7ccccc7)c6c45)n3)cc2)cc1] | 81 |
| 1-158 | 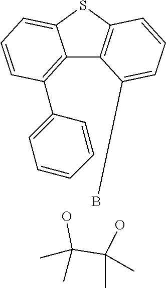 | 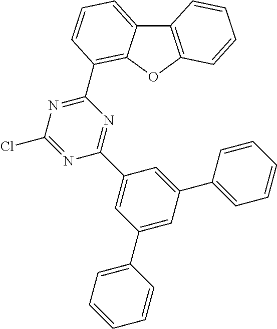 [SMILES: Clc1nc(-c2cc(-c3ccccc3)cc(-c3ccccc3)c2)nc(-c2cccc3c2oc2ccccc23)n1] | 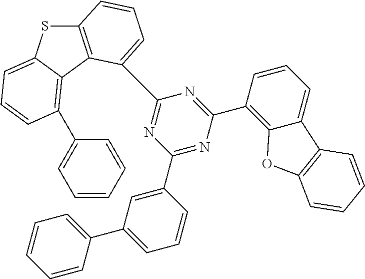 [SMILES: c1ccc(-c2cccc(-c3nc(-c4cccc5c4oc4ccccc45)nc(-c4cccc5sc6cccc(-c7ccccc7)c6c45)n3)c2)cc1] | 82 |
| 1-159 | 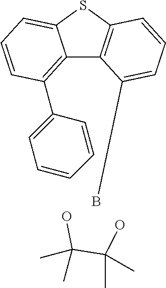 | 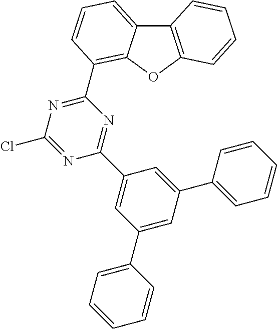 [SMILES: Clc1nc(-c2cc(-c3ccccc3)cc(-c3ccccc3)c2)nc(-c2cccc3c2oc2ccccc23)n1] | 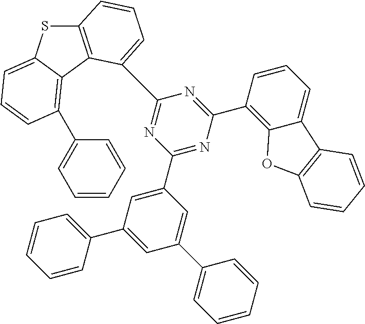 [SMILES: c1ccc(-c2cc(-c3ccccc3)cc(-c3nc(-c4cccc5c4oc4ccccc45)nc(-c4cccc5sc6cccc(-c7ccccc7)c6c45)n3)c2)cc1] | 83 |
| 1-160 | 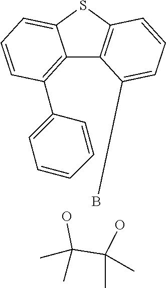 | 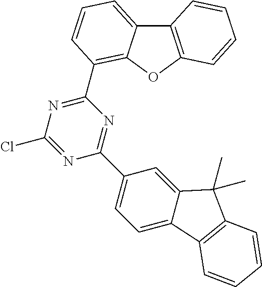 [SMILES: CC1(C)c2ccccc2-c2ccc(-c3nc(Cl)nc(-c4cccc5c4oc4ccccc45)n3)cc21] | 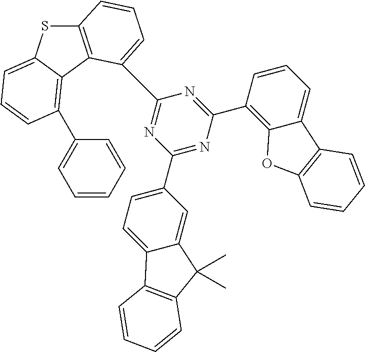 [SMILES: CC1(C)c2ccccc2-c2ccc(-c3nc(-c4cccc5c4oc4ccccc45)nc(-c4cccc5sc6cccc(-c7ccccc7)c6c45)n3)cc21] | 88 |
| 1-161 | 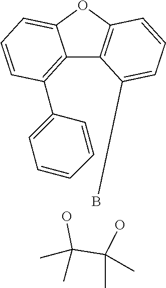 | 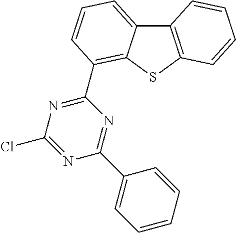 [SMILES: Clc1nc(-c2ccccc2)nc(-c2cccc3c2sc2ccccc23)n1] | 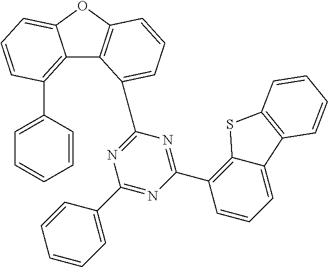 [SMILES: c1ccc(-c2nc(-c3cccc4c3sc3ccccc34)nc(-c3cccc4oc5cccc(-c6ccccc6)c5c34)n2)cc1] | 85 |
| 1-162 | 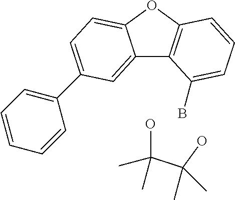 | 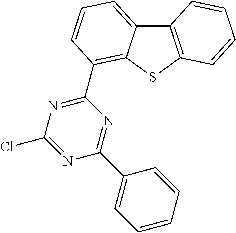 [SMILES: Clc1nc(-c2ccccc2)nc(-c2cccc3c2sc2ccccc23)n1] | 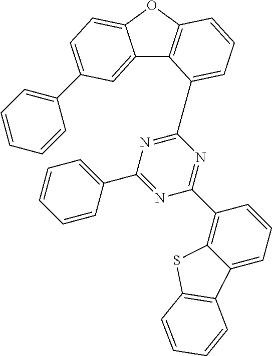 [SMILES: c1ccc(-c2ccc3oc4cccc(-c5nc(-c6ccccc6)nc(-c6cccc7c6sc6ccccc67)n5)c4c3c2)cc1] | 83 |
| 1-163 | 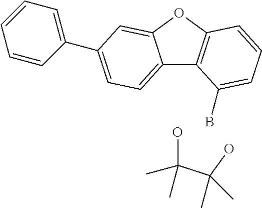 | 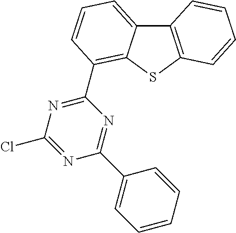 [SMILES: Clc1nc(-c2ccccc2)nc(-c2cccc3c2sc2ccccc23)n1] | 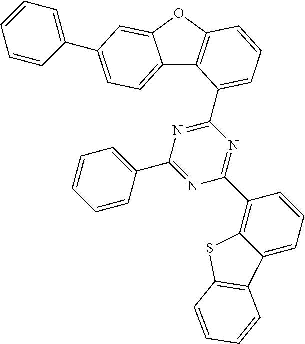 [SMILES: c1ccc(-c2ccc3c(c2)oc2cccc(-c4nc(-c5ccccc5)nc(-c5cccc6c5sc5ccccc56)n4)c23)cc1] | 84 |
| 1-164 | 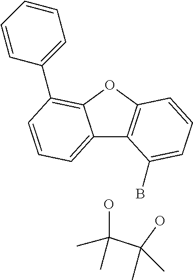 | 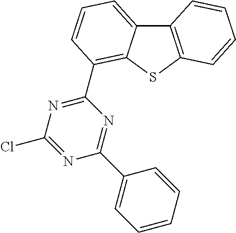 [SMILES: Clc1nc(-c2ccccc2)nc(-c2cccc3c2sc2ccccc23)n1] | 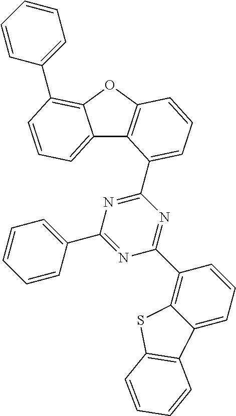 [SMILES: c1ccc(-c2nc(-c3cccc4c3sc3ccccc34)nc(-c3cccc4oc5c(-c6ccccc6)cccc5c34)n2)cc1] | 83 |
| 1-165 | 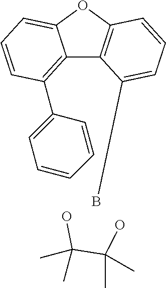 | 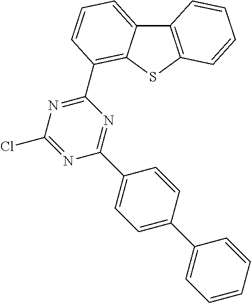 [SMILES: Clc1nc(-c2ccc(-c3ccccc3)cc2)nc(-c2cccc3c2sc2ccccc23)n1] | 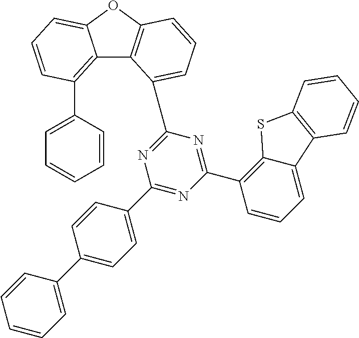 [SMILES: c1ccc(-c2ccc(-c3nc(-c4cccc5c4sc4ccccc45)nc(-c4cccc5oc6cccc(-c7ccccc7)c6c45)n3)cc2)cc1] | 80 |
| 1-166 | 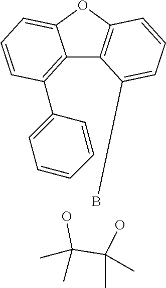 | 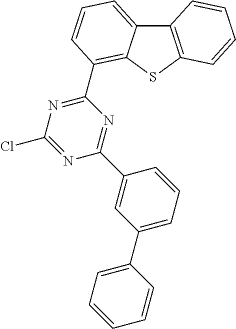 [SMILES: Clc1nc(-c2cccc(-c3ccccc3)c2)nc(-c2cccc3c2sc2ccccc23)n1] | 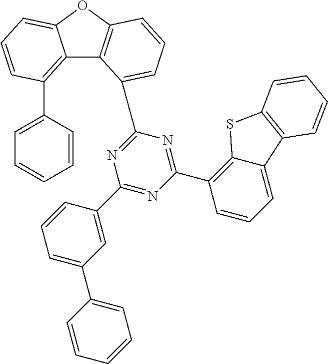 [SMILES: c1ccc(-c2cccc(-c3nc(-c4cccc5c4sc4ccccc45)nc(-c4cccc5oc6cccc(-c7ccccc7)c6c45)n3)c2)cc1] | 88 |
| 1-167 | 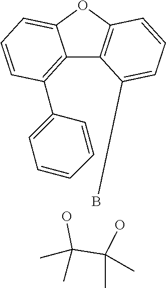 | 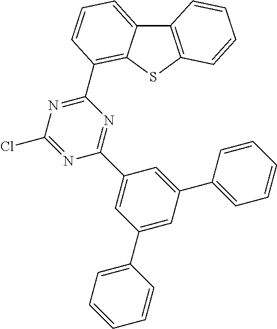 [SMILES: Clc1nc(-c2cc(-c3ccccc3)cc(-c3ccccc3)c2)nc(-c2cccc3c2sc2ccccc23)n1] | 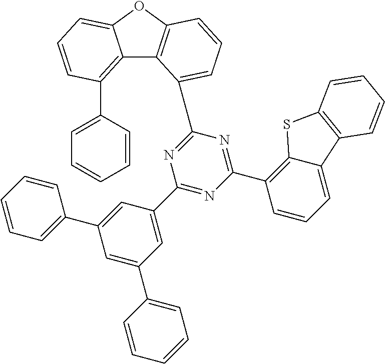 [SMILES: c1ccc(-c2cc(-c3ccccc3)cc(-c3nc(-c4cccc5c4sc4ccccc45)nc(-c4cccc5oc6cccc(-c7ccccc7)c6c45)n3)c2)cc1] | 86 |
| 1-168 | 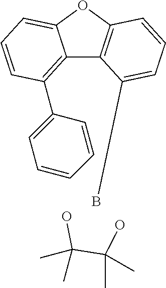 | 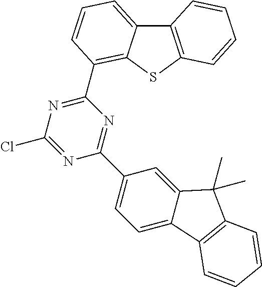 [SMILES: CC1(C)c2ccccc2-c2ccc(-c3nc(Cl)nc(-c4cccc5c4sc4ccccc45)n3)cc21] | 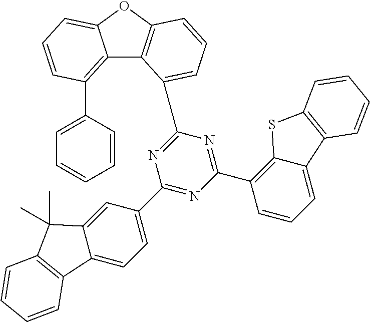 [SMILES: CC1(C)c2ccccc2-c2ccc(-c3nc(-c4cccc5c4sc4ccccc45)nc(-c4cccc5oc6cccc(-c7ccccc7)c6c45)n3)cc21] | 85 |
| 1-169 | 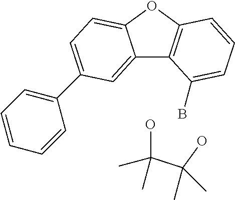 | 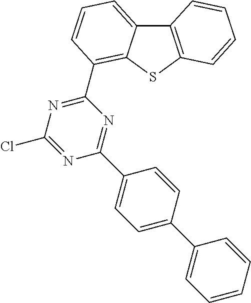 [SMILES: Clc1nc(-c2ccc(-c3ccccc3)cc2)nc(-c2cccc3c2sc2ccccc23)n1] | 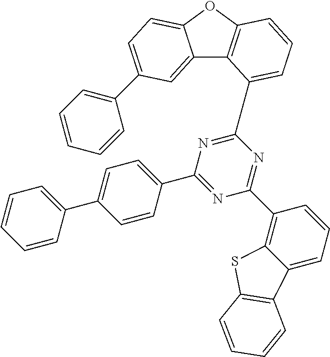 [SMILES: c1ccc(-c2ccc(-c3nc(-c4cccc5c4sc4ccccc45)nc(-c4cccc5oc6ccc(-c7ccccc7)cc6c45)n3)cc2)cc1] | 83 |
| 1-170 | 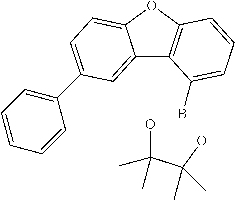 | 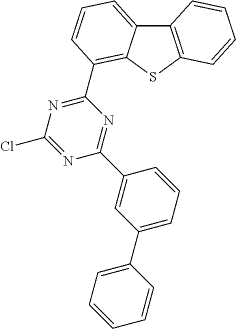 [SMILES: Clc1nc(-c2cccc(-c3ccccc3)c2)nc(-c2cccc3c2sc2ccccc23)n1] | 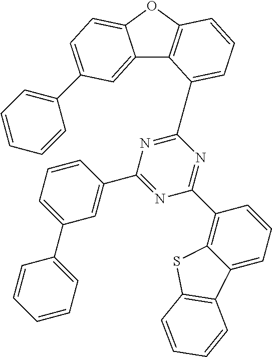 [SMILES: c1ccc(-c2cccc(-c3nc(-c4cccc5c4sc4ccccc45)nc(-c4cccc5oc6ccc(-c7ccccc7)cc6c45)n3)c2)cc1] | 84 |
| 1-171 | 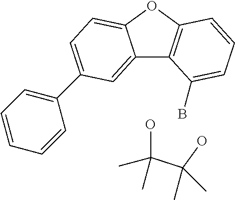 | 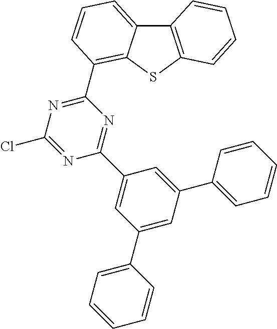 [SMILES: Clc1nc(-c2cc(-c3ccccc3)cc(-c3ccccc3)c2)nc(-c2cccc3c2sc2ccccc23)n1] | 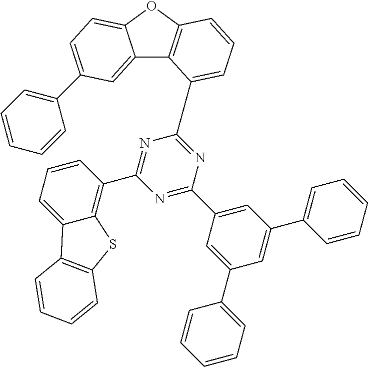 [SMILES: c1ccc(-c2cc(-c3ccccc3)cc(-c3nc(-c4cccc5c4sc4ccccc45)nc(-c4cccc5oc6ccc(-c7ccccc7)cc6c45)n3)c2)cc1] | 88 |
| 1-172 | 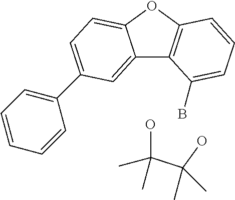 | 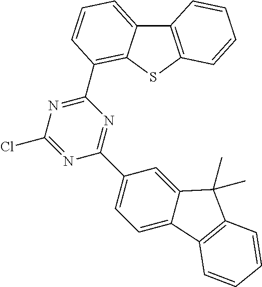 [SMILES: CC1(C)c2ccccc2-c2ccc(-c3nc(Cl)nc(-c4cccc5c4sc4ccccc45)n3)cc21] | 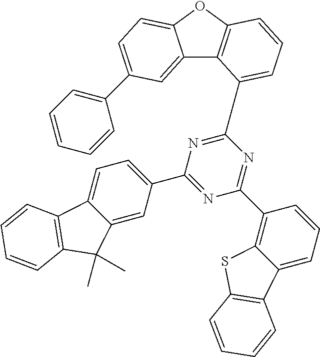 [SMILES: CC1(C)c2ccccc2-c2ccc(-c3nc(-c4cccc5c4sc4ccccc45)nc(-c4cccc5oc6ccc(-c7ccccc7)cc6c45)n3)cc21] | 86 |
| 1-173 | 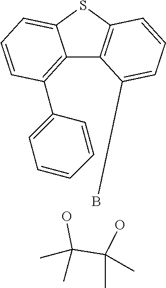 | 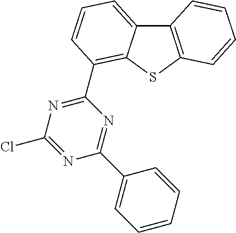 [SMILES: Clc1nc(-c2ccccc2)nc(-c2cccc3c2sc2ccccc23)n1] | 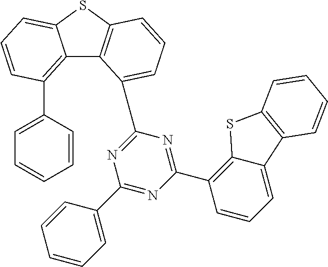 [SMILES: c1ccc(-c2nc(-c3cccc4c3sc3ccccc34)nc(-c3cccc4sc5cccc(-c6ccccc6)c5c34)n2)cc1] | 84 |
| 1-174 | 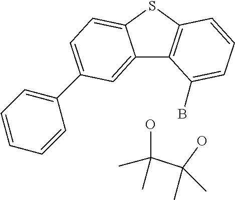 | 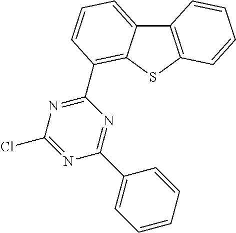 [SMILES: Clc1nc(-c2ccccc2)nc(-c2cccc3c2sc2ccccc23)n1] | 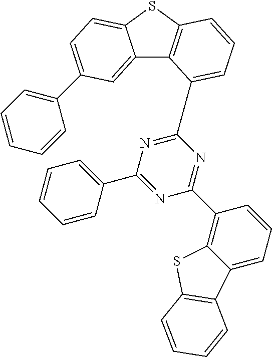 [SMILES: c1ccc(-c2ccc3sc4cccc(-c5nc(-c6ccccc6)nc(-c6cccc7c6sc6ccccc67)n5)c4c3c2)cc1] | 85 |
| 1-175 | 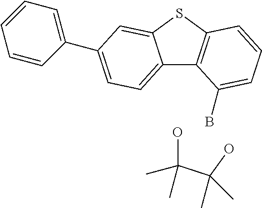 | 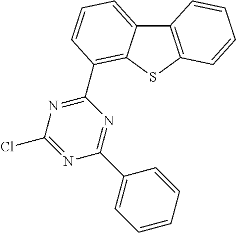 [SMILES: Clc1nc(-c2ccccc2)nc(-c2cccc3c2sc2ccccc23)n1] | 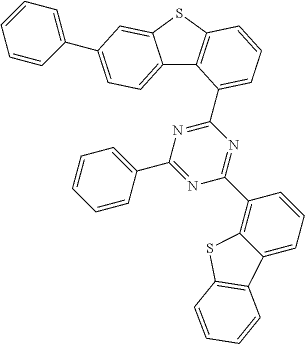 [SMILES: c1ccc(-c2ccc3c(c2)sc2cccc(-c4nc(-c5ccccc5)nc(-c5cccc6c5sc5ccccc56)n4)c23)cc1] | 83 |
| 1-176 | 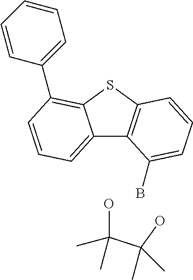 | 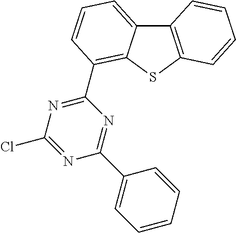 [SMILES: Clc1nc(-c2ccccc2)nc(-c2cccc3c2sc2ccccc23)n1] | 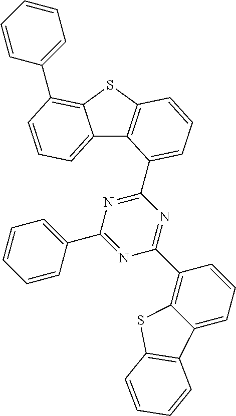 [SMILES: c1ccc(-c2nc(-c3cccc4c3sc3ccccc34)nc(-c3cccc4sc5c(-c6ccccc6)cccc5c34)n2)cc1] | 85 |
| 1-177 | 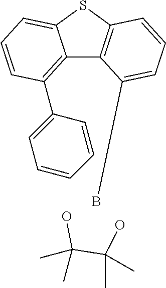 | 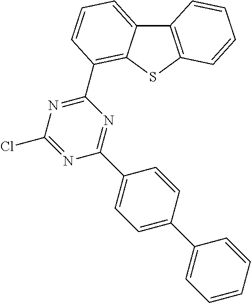 [SMILES: Clc1nc(-c2ccc(-c3ccccc3)cc2)nc(-c2cccc3c2sc2ccccc23)n1] | 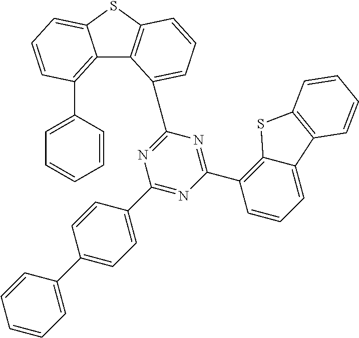 [SMILES: c1ccc(-c2ccc(-c3nc(-c4cccc5c4sc4ccccc45)nc(-c4cccc5sc6cccc(-c7ccccc7)c6c45)n3)cc2)cc1] | 83 |
| 1-178 | 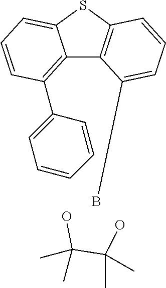 | 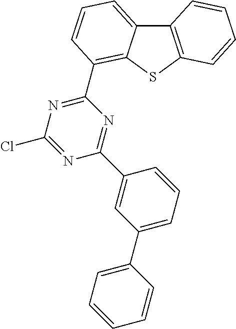 [SMILES: Clc1nc(-c2cccc(-c3ccccc3)c2)nc(-c2cccc3c2sc2ccccc23)n1] | 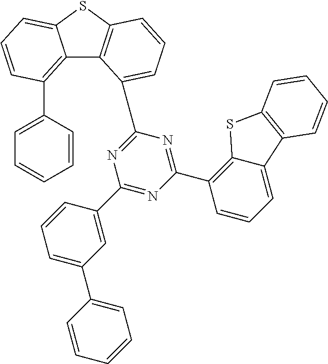 [SMILES: c1ccc(-c2cccc(-c3nc(-c4cccc5c4sc4ccccc45)nc(-c4cccc5sc6cccc(-c7ccccc7)c6c45)n3)c2)cc1] | 80 |
| 1-179 | 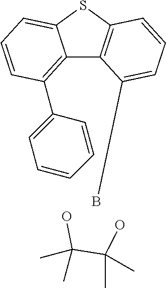 | 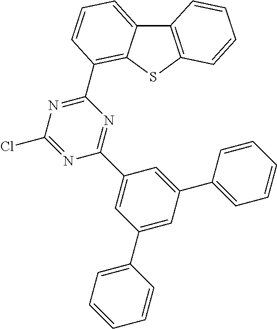 [SMILES: Clc1nc(-c2cc(-c3ccccc3)cc(-c3ccccc3)c2)nc(-c2cccc3c2sc2ccccc23)n1] | 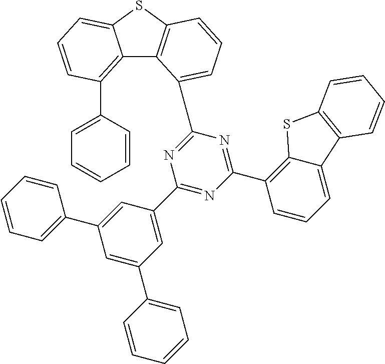 [SMILES: c1ccc(-c2cc(-c3ccccc3)cc(-c3nc(-c4cccc5c4sc4ccccc45)nc(-c4cccc5sc6cccc(-c7ccccc7)c6c45)n3)c2)cc1] | 81 |
| 1-180 | 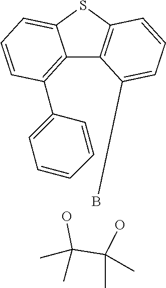 | 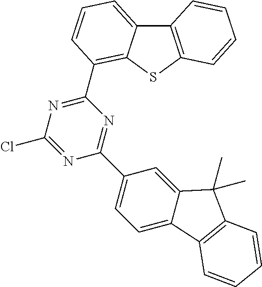 [SMILES: CC1(C)c2ccccc2-c2ccc(-c3nc(Cl)nc(-c4cccc5c4sc4ccccc45)n3)cc21] | 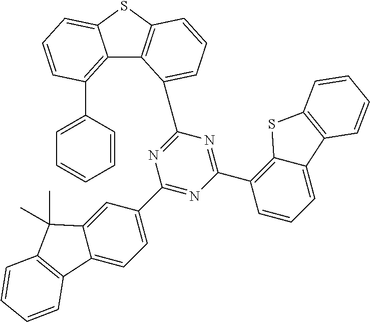 [SMILES: CC1(C)c2ccccc2-c2ccc(-c3nc(-c4cccc5c4sc4ccccc45)nc(-c4cccc5sc6cccc(-c7ccccc7)c6c45)n3)cc21] | 82 |

### <Preparation Example 2> Preparation of Compound 1-81 (F)

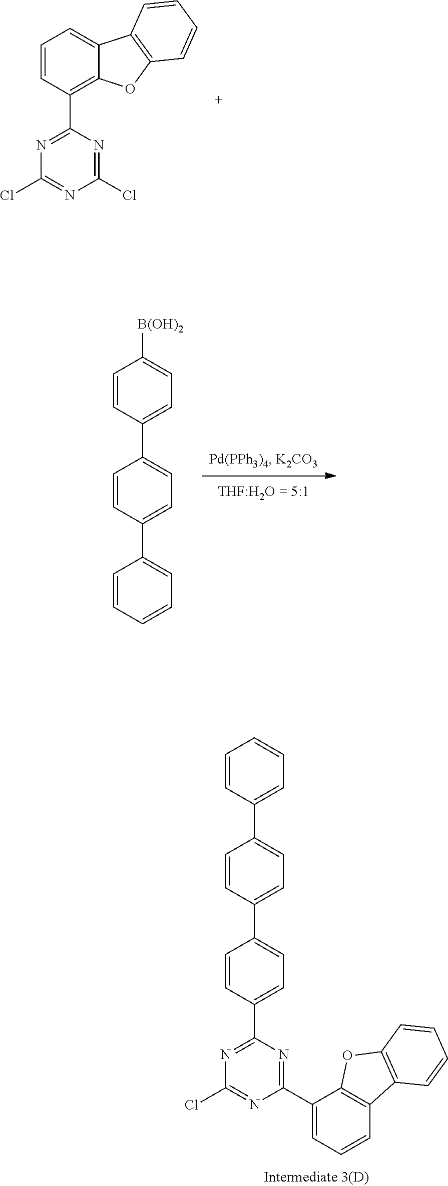 [SMILES: Clc1nc(Cl)nc(-c2cccc3c2oc2ccccc23)n1 ; OB(O)c1ccc(-c2ccc(-c3ccccc3)cc2)cc1 ; Clc1nc(-c2ccc(-c3ccc(-c4ccccc4)cc3)cc2)nc(-c2cccc3c2oc2ccccc23)n1]

### Synthesis Method of Intermediate 3(D)

After [1,1′:4′,1″-terphenyl]-4-ylboronic acid (8.6 g, 31.6 mmol), 2,4-dichloro-6-(dibenzo[b,d]furan-4-yl)-1,3,5-triazine (10 g, 31.6 mmol), tetrakis (triphenylphosphine)palladium(0) (Pd(PPh₃)₄) (1.8 g, 1.6 mmol), and K₂CO₃ (13 g, 94.8 mmol) were put into a reaction flask, a mixture of THF/water (100 ml/20 ml) was heated at 85° C. for 4 hours. After the reaction was completed, the temperature was lowered to room temperature, and then the resulting solid was washed with distilled water and methanol (MeOH) to obtain Intermediate 3 (D). (13 g, 81%)

### Synthesis Method of Intermediate 1(A)

Intermediate 1(A) was synthesized in the same manner as in Preparation Example 1.

### Synthesis Method of Compound 1-81(F)

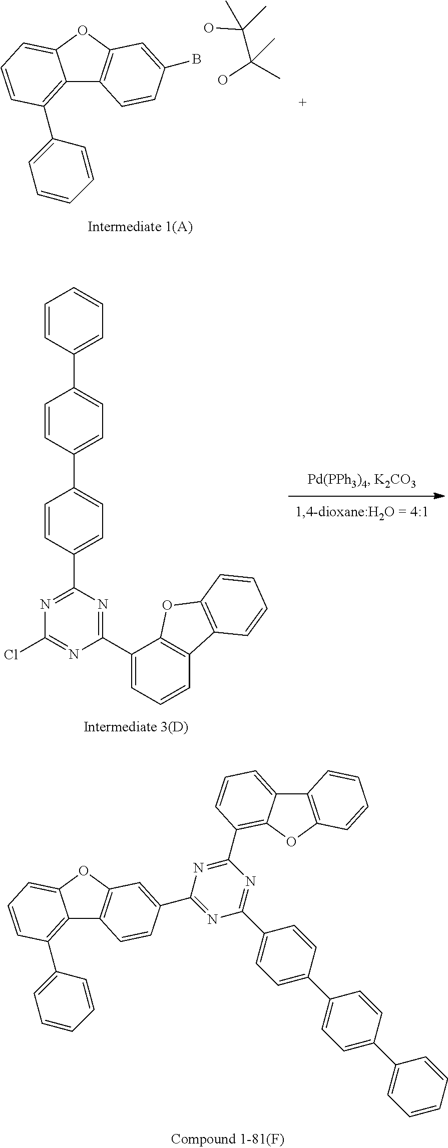

A mixture of Intermediate 1 (A) (10 g, 27 mmol), Intermediate 3(D) (16.5 g, 32.4 mmol), tetrakis (triphenylphosphine)palladium(0) (Pd(PPh₃)₄) (1.6 g, 1.4 mmol), K₂CO₃ (11.2 g, 81 mmol), and 1,4-dioxane/water (100 ml/25 ml) was refluxed at 120° C. in a reaction flask for 3 hours. After the reaction was completed, the temperature was lowered to room temperature, and then the resulting solid was washed with distilled water and methanol (MeOH) to obtain Compound 1-81(F). (15 g, 79%)

The following Compound (F) was synthesized in the same manner as in Preparation Example 2 using compounds in the following Table 2 instead of Intermediate 1 (A) and Intermediate 3(D) in Preparation Example 2.

TABLE 2

| Com- | | | | |
|---|---|---|---|---|
| pound | Intermediate | Intermediate | | Yield |
| No. | 1 (A) | 3 (D) | Compound (F) | (%) |
| 1-82 | 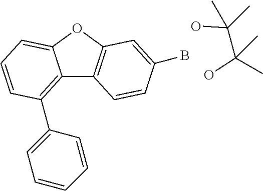 | 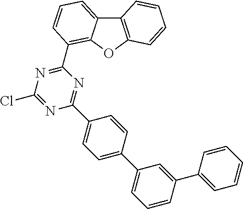 [SMILES: Clc1nc(-c2ccc(-c3cccc(-c4ccccc4)c3)cc2)nc(-c2cccc3c2oc2ccccc23)n1] | 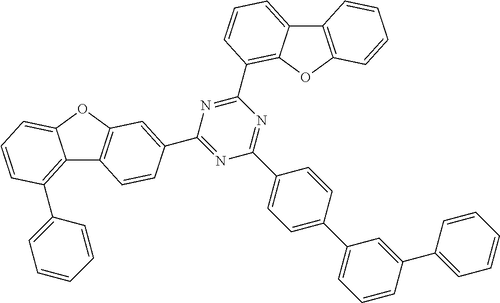 [SMILES: c1ccc(-c2cccc(-c3ccc(-c4nc(-c5ccc6c(c5)oc5cccc(-c7ccccc7)c56)nc(-c5cccc6c5oc5ccccc56)n4)cc3)c2)cc1] | 85 |
| 1-83 | 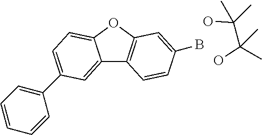 | 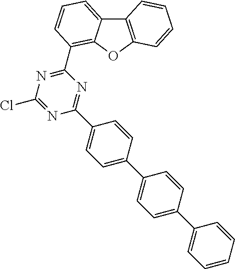 [SMILES: Clc1nc(-c2ccc(-c3ccc(-c4ccccc4)cc3)cc2)nc(-c2cccc3c2oc2ccccc23)n1] | 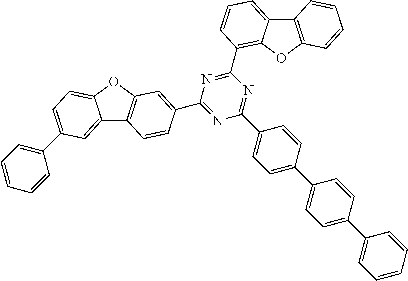 [SMILES: c1ccc(-c2ccc(-c3ccc(-c4nc(-c5ccc6c(c5)oc5ccc(-c7ccccc7)cc56)nc(-c5cccc6c5oc5ccccc56)n4)cc3)cc2)cc1] | 83 |
| 1-84 | 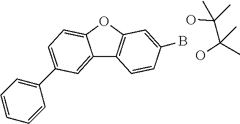 | 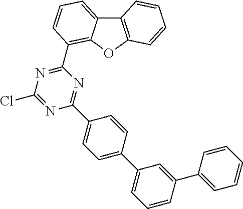 [SMILES: Clc1nc(-c2ccc(-c3cccc(-c4ccccc4)c3)cc2)nc(-c2cccc3c2oc2ccccc23)n1] | 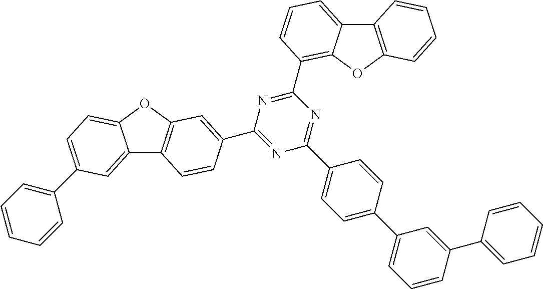 [SMILES: c1ccc(-c2cccc(-c3ccc(-c4nc(-c5ccc6c(c5)oc5ccc(-c7ccccc7)cc56)nc(-c5cccc6c5oc5ccccc56)n4)cc3)c2)cc1] | 80 |
| 1-85 | 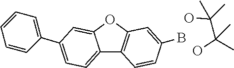 | 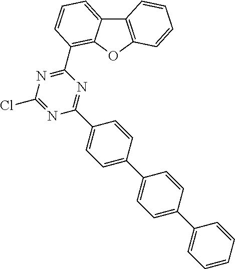 [SMILES: Clc1nc(-c2ccc(-c3ccc(-c4ccccc4)cc3)cc2)nc(-c2cccc3c2oc2ccccc23)n1] | 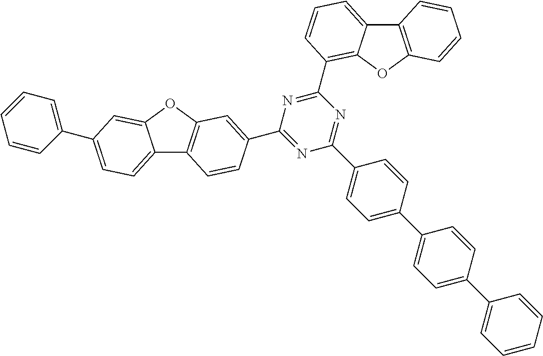 [SMILES: c1ccc(-c2ccc(-c3ccc(-c4nc(-c5ccc6c(c5)oc5cc(-c7ccccc7)ccc56)nc(-c5cccc6c5oc5ccccc56)n4)cc3)cc2)cc1] | 81 |
| 1-86 | 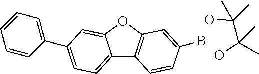 | 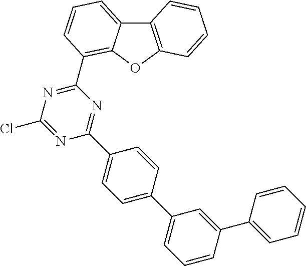 [SMILES: Clc1nc(-c2ccc(-c3cccc(-c4ccccc4)c3)cc2)nc(-c2cccc3c2oc2ccccc23)n1] | 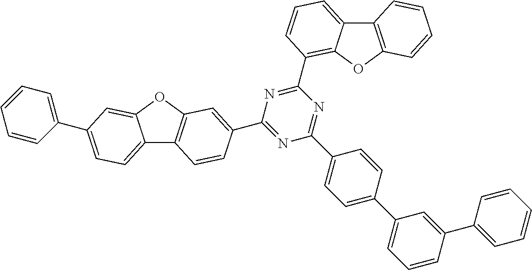 [SMILES: c1ccc(-c2cccc(-c3ccc(-c4nc(-c5ccc6c(c5)oc5cc(-c7ccccc7)ccc56)nc(-c5cccc6c5oc5ccccc56)n4)cc3)c2)cc1] | 82 |
| 1-87 | 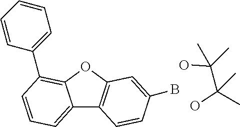 | 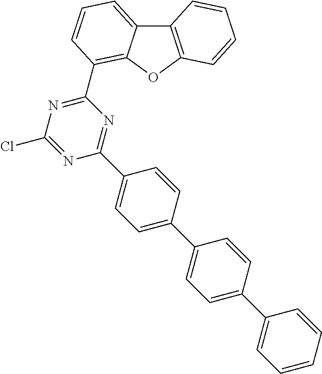 [SMILES: Clc1nc(-c2ccc(-c3ccc(-c4ccccc4)cc3)cc2)nc(-c2cccc3c2oc2ccccc23)n1] | 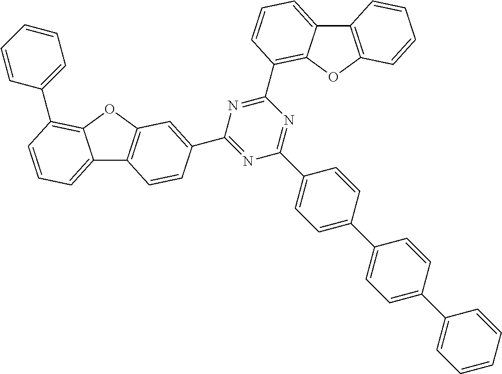 [SMILES: c1ccc(-c2ccc(-c3ccc(-c4nc(-c5ccc6c(c5)oc5c(-c7ccccc7)cccc56)nc(-c5cccc6c5oc5ccccc56)n4)cc3)cc2)cc1] | 83 |
| 1-88 | 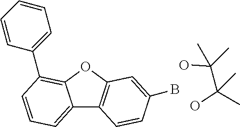 | 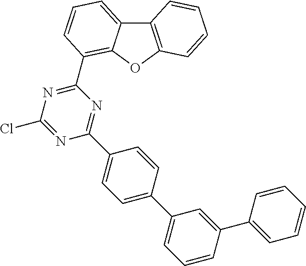 [SMILES: Clc1nc(-c2ccc(-c3cccc(-c4ccccc4)c3)cc2)nc(-c2cccc3c2oc2ccccc23)n1] | 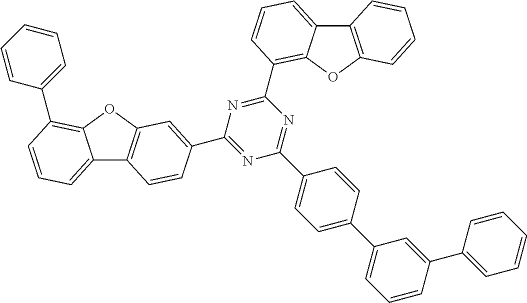 [SMILES: c1ccc(-c2cccc(-c3ccc(-c4nc(-c5ccc6c(c5)oc5c(-c7ccccc7)cccc56)nc(-c5cccc6c5oc5ccccc56)n4)cc3)c2)cc1] | 88 |
| 1-89 | 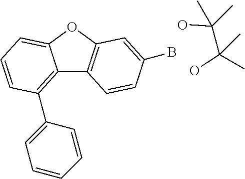 | 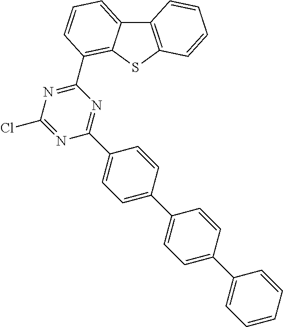 [SMILES: Clc1nc(-c2ccc(-c3ccc(-c4ccccc4)cc3)cc2)nc(-c2cccc3c2sc2ccccc23)n1] | 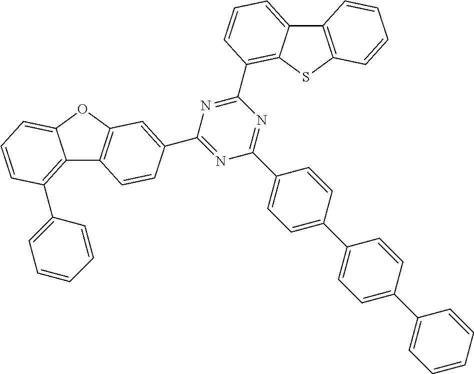 [SMILES: c1ccc(-c2ccc(-c3ccc(-c4nc(-c5ccc6c(c5)oc5cccc(-c7ccccc7)c56)nc(-c5cccc6c5sc5ccccc56)n4)cc3)cc2)cc1] | 85 |
| 1-90 | 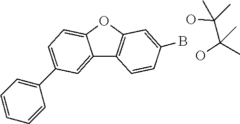 | 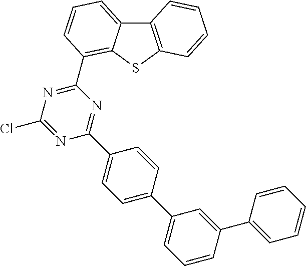 [SMILES: Clc1nc(-c2ccc(-c3cccc(-c4ccccc4)c3)cc2)nc(-c2cccc3c2sc2ccccc23)n1] | 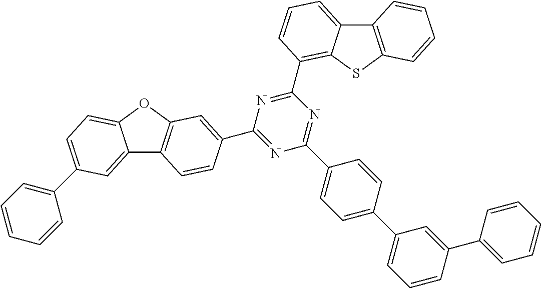 [SMILES: c1ccc(-c2cccc(-c3ccc(-c4nc(-c5ccc6c(c5)oc5ccc(-c7ccccc7)cc56)nc(-c5cccc6c5sc5ccccc56)n4)cc3)c2)cc1] | 83 |
| 1-91 | 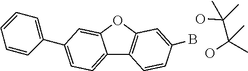 | 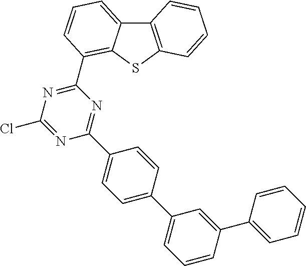 [SMILES: Clc1nc(-c2ccc(-c3cccc(-c4ccccc4)c3)cc2)nc(-c2cccc3c2sc2ccccc23)n1] | 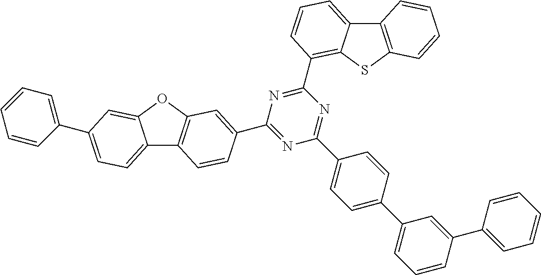 [SMILES: c1ccc(-c2cccc(-c3ccc(-c4nc(-c5ccc6c(c5)oc5cc(-c7ccccc7)ccc56)nc(-c5cccc6c5sc5ccccc56)n4)cc3)c2)cc1] | 84 |
| 1-92 | 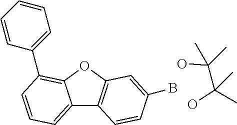 | 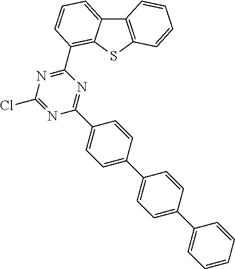 [SMILES: Clc1nc(-c2ccc(-c3ccc(-c4ccccc4)cc3)cc2)nc(-c2cccc3c2sc2ccccc23)n1] | 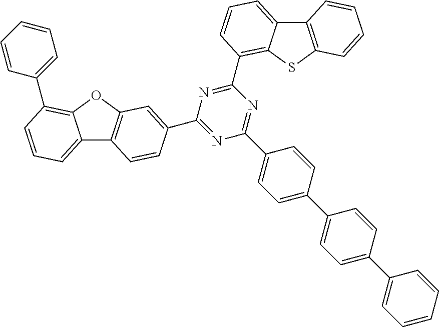 [SMILES: c1ccc(-c2ccc(-c3ccc(-c4nc(-c5ccc6c(c5)oc5c(-c7ccccc7)cccc56)nc(-c5cccc6c5sc5ccccc56)n4)cc3)cc2)cc1] | 83 |
| 1-93 | 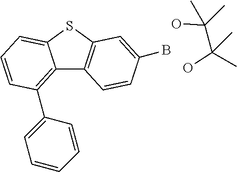 | 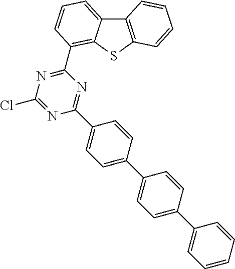 [SMILES: Clc1nc(-c2ccc(-c3ccc(-c4ccccc4)cc3)cc2)nc(-c2cccc3c2sc2ccccc23)n1] | 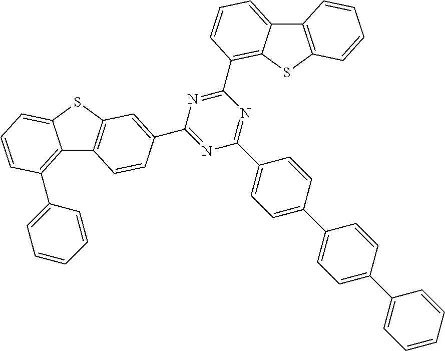 [SMILES: c1ccc(-c2ccc(-c3ccc(-c4nc(-c5ccc6c(c5)sc5cccc(-c7ccccc7)c56)nc(-c5cccc6c5sc5ccccc56)n4)cc3)cc2)cc1] | 80 |
| 1-94 | 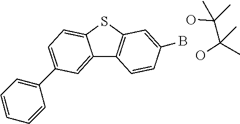 | 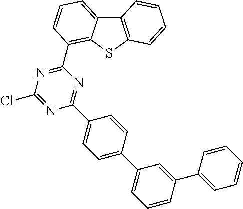 [SMILES: Clc1nc(-c2ccc(-c3cccc(-c4ccccc4)c3)cc2)nc(-c2cccc3c2sc2ccccc23)n1] | 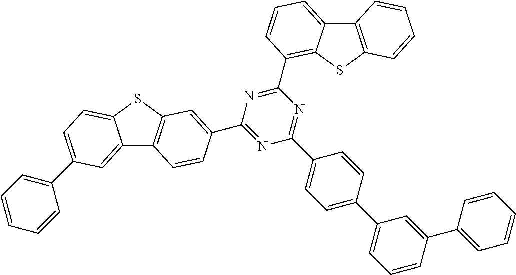 [SMILES: c1ccc(-c2cccc(-c3ccc(-c4nc(-c5ccc6c(c5)sc5ccc(-c7ccccc7)cc56)nc(-c5cccc6c5sc5ccccc56)n4)cc3)c2)cc1] | 88 |
| 1-95 | 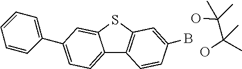 | 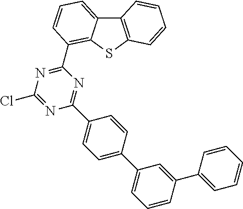 [SMILES: Clc1nc(-c2ccc(-c3cccc(-c4ccccc4)c3)cc2)nc(-c2cccc3c2sc2ccccc23)n1] | 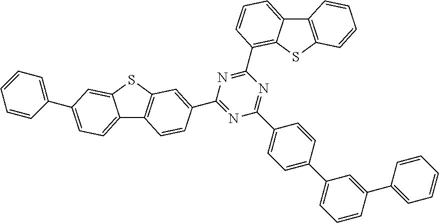 [SMILES: c1ccc(-c2cccc(-c3ccc(-c4nc(-c5ccc6c(c5)sc5cc(-c7ccccc7)ccc56)nc(-c5cccc6c5sc5ccccc56)n4)cc3)c2)cc1] | 86 |
| 1-96 | 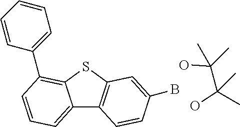 | 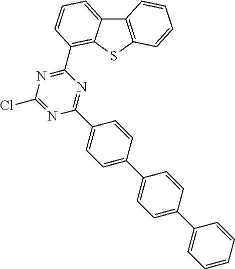 [SMILES: Clc1nc(-c2ccc(-c3ccc(-c4ccccc4)cc3)cc2)nc(-c2cccc3c2sc2ccccc23)n1] | 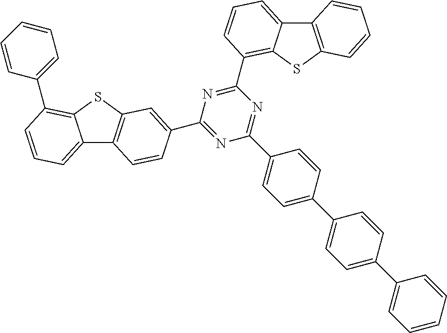 [SMILES: c1ccc(-c2ccc(-c3ccc(-c4nc(-c5ccc6c(c5)sc5c(-c7ccccc7)cccc56)nc(-c5cccc6c5sc5ccccc56)n4)cc3)cc2)cc1] | 85 |
| 1-97 | 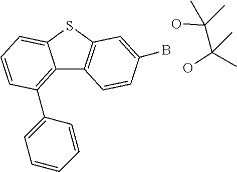 | 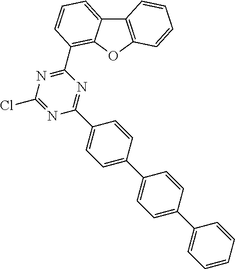 [SMILES: Clc1nc(-c2ccc(-c3ccc(-c4ccccc4)cc3)cc2)nc(-c2cccc3c2oc2ccccc23)n1] | 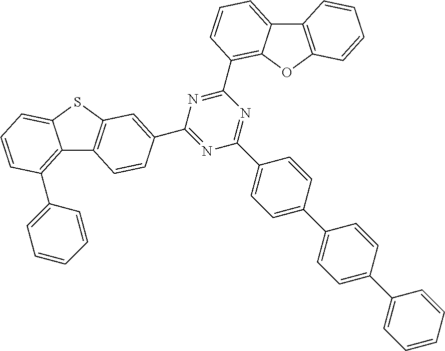 [SMILES: c1ccc(-c2ccc(-c3ccc(-c4nc(-c5ccc6c(c5)sc5cccc(-c7ccccc7)c56)nc(-c5cccc6c5oc5ccccc56)n4)cc3)cc2)cc1] | 83 |
| 1-98 | 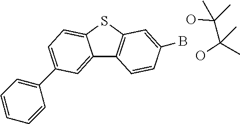 | 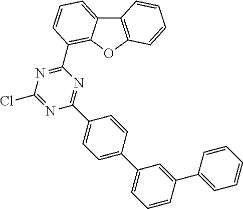 [SMILES: Clc1nc(-c2ccc(-c3cccc(-c4ccccc4)c3)cc2)nc(-c2cccc3c2oc2ccccc23)n1] | 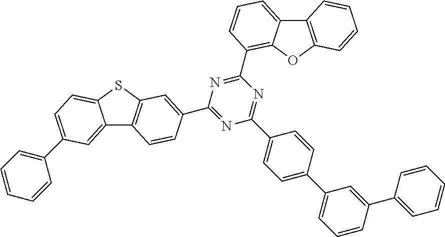 [SMILES: c1ccc(-c2cccc(-c3ccc(-c4nc(-c5ccc6c(c5)sc5ccc(-c7ccccc7)cc56)nc(-c5cccc6c5oc5ccccc56)n4)cc3)c2)cc1] | 84 |
| 1-99 | 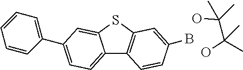 | 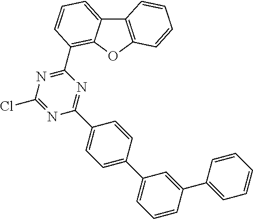 [SMILES: Clc1nc(-c2ccc(-c3cccc(-c4ccccc4)c3)cc2)nc(-c2cccc3c2oc2ccccc23)n1] | 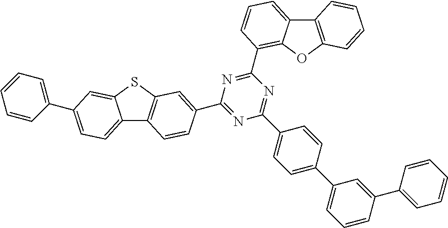 [SMILES: c1ccc(-c2cccc(-c3ccc(-c4nc(-c5ccc6c(c5)sc5cc(-c7ccccc7)ccc56)nc(-c5cccc6c5oc5ccccc56)n4)cc3)c2)cc1] | 88 |
| 1-100 | 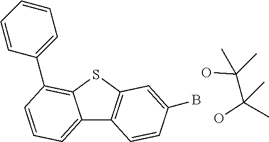 | 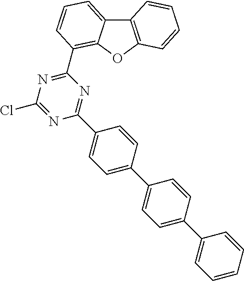 [SMILES: Clc1nc(-c2ccc(-c3ccc(-c4ccccc4)cc3)cc2)nc(-c2cccc3c2oc2ccccc23)n1] | 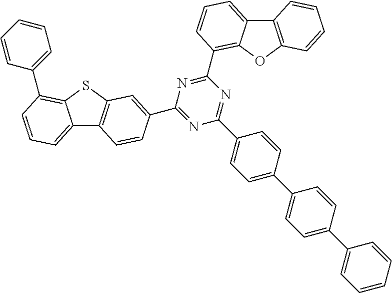 [SMILES: c1ccc(-c2ccc(-c3ccc(-c4nc(-c5ccc6c(c5)sc5c(-c7ccccc7)cccc56)nc(-c5cccc6c5oc5ccccc56)n4)cc3)cc2)cc1] | 86 |

### <Preparation Example 3> Preparation of Compound 1-181(G)

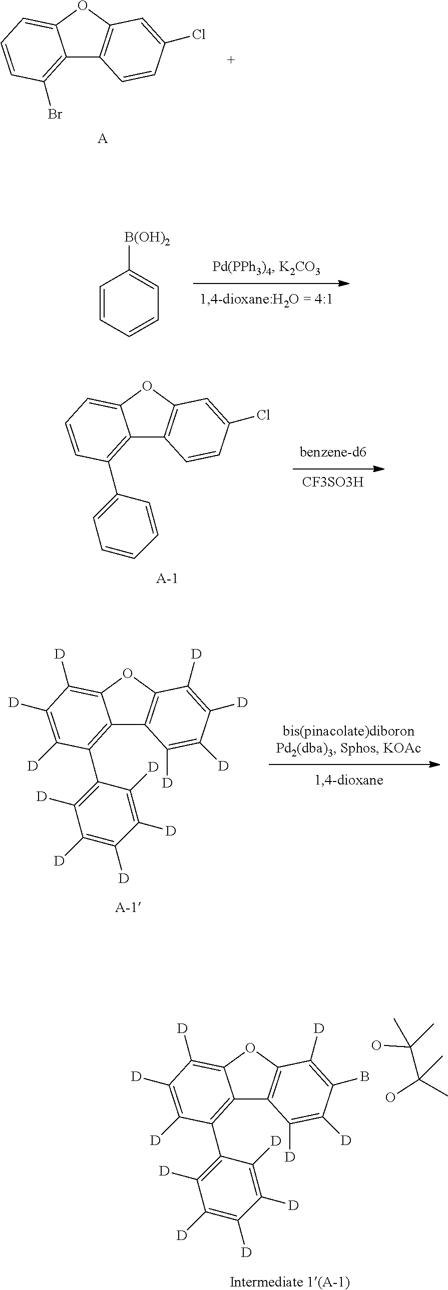

### Synthesis Method of Compound A-1

Compound A-1 was synthesized in the same manner as in Preparation Example 1.

### Synthesis Method of Compound A-1′

Compound A-1 (1 g, 1 eq.), benzene-D₆ (50 g, 210.8 eq.), and CF₃SO₃H (14.4 g, 32.4 eq.) were put into a reaction flask, and then reacted at 50° C. for 1 hour. After the reaction was completed, H₂O was added thereto for neutralization, then the resulting product was extracted with MC and H₂O, and then the organic material layer was column-purified to obtain Compound A-1′. (0.7 g, 62%)

### Synthesis Method of Intermediate 1′(A-1)

Intermediate 1′(A-1) was synthesized in the same manner as in Intermediate 1(A) of Preparation Example 1.

### Synthesis Method of Compound 1-181(G)

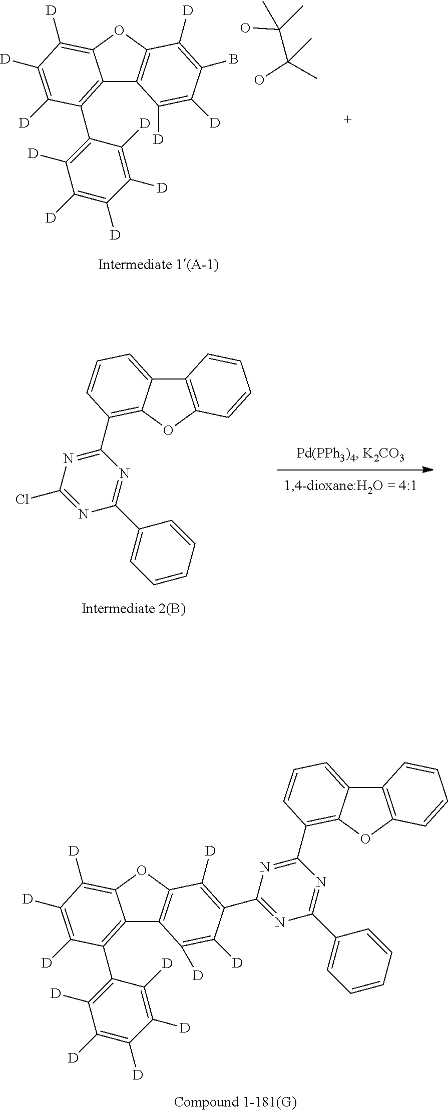

Compound 1-181(G) was synthesized in the same manner as in Compound 1-1(C) of Preparation Example 1.

The following Compound (G) was synthesized in the same manner as in Preparation Example 3 using compounds in the following Table 3 instead of Intermediate 1 (A-1) and Intermediate 2(B) in Preparation Example 3.

TABLE 3

| Com- | | | | |
|---|---|---|---|---|
| pound | Intermediate | Intermediate | | Yield |
| No. | 1′ (A-1) | 2 (B) | Compound (G) | (%) |
| 1-183 | 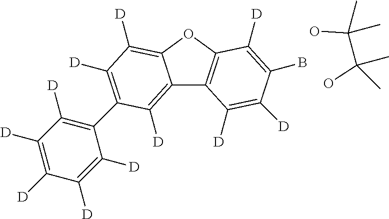 | 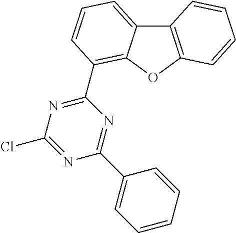 [SMILES: Clc1nc(-c2ccccc2)nc(-c2cccc3c2oc2ccccc23)n1] | 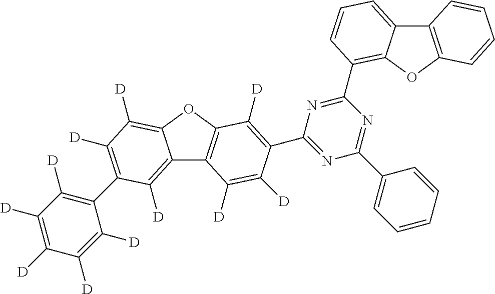 [SMILES: [2H]c1c([2H])c([2H])c(-c2c([2H])c([2H])c3oc4c([2H])c(-c5nc(-c6ccccc6)nc(-c6cccc7c6oc6ccccc67)n5)c([2H])c([2H])c4c3c2[2H])c([2H])c1[2H]] | 85 |
| 1-185 | 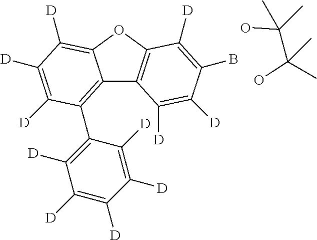 | 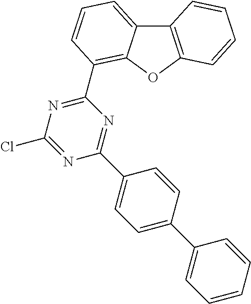 [SMILES: Clc1nc(-c2ccc(-c3ccccc3)cc2)nc(-c2cccc3c2oc2ccccc23)n1] | 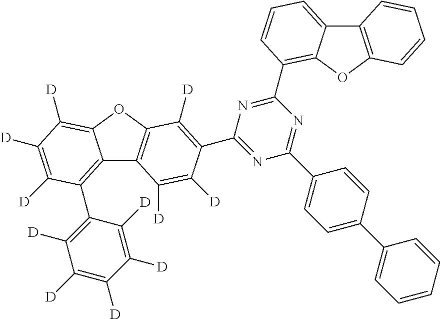 [SMILES: [2H]c1c([2H])c([2H])c(-c2c([2H])c([2H])c([2H])c3oc4c([2H])c(-c5nc(-c6ccc(-c7ccccc7)cc6)nc(-c6cccc7c6oc6ccccc67)n5)c([2H])c([2H])c4c23)c([2H])c1[2H]] | 83 |
| 1-187 | 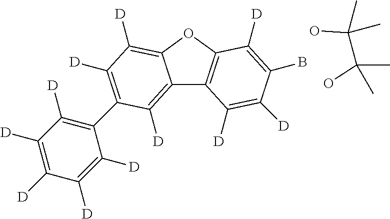 | 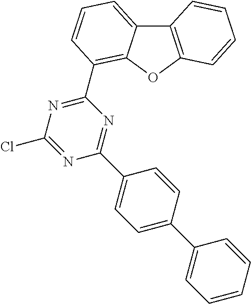 [SMILES: Clc1nc(-c2ccc(-c3ccccc3)cc2)nc(-c2cccc3c2oc2ccccc23)n1] | 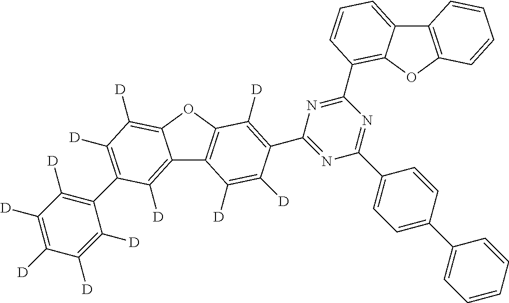 [SMILES: [2H]c1c([2H])c([2H])c(-c2c([2H])c([2H])c3oc4c([2H])c(-c5nc(-c6ccc(-c7ccccc7)cc6)nc(-c6cccc7c6oc6ccccc67)n5)c([2H])c([2H])c4c3c2[2H])c([2H])c1[2H]] | 80 |
| 1-188 | 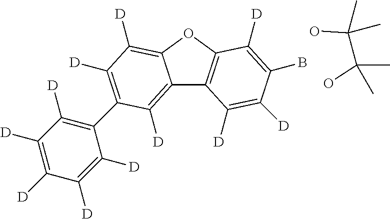 | 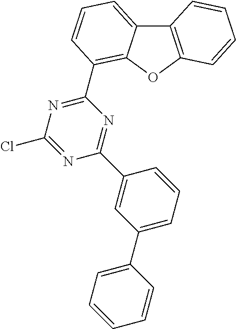 [SMILES: Clc1nc(-c2cccc(-c3ccccc3)c2)nc(-c2cccc3c2oc2ccccc23)n1] | 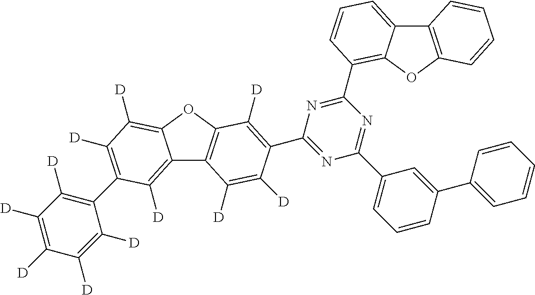 [SMILES: [2H]c1c([2H])c([2H])c(-c2c([2H])c([2H])c3oc4c([2H])c(-c5nc(-c6cccc(-c7ccccc7)c6)nc(-c6cccc7c6oc6ccccc67)n5)c([2H])c([2H])c4c3c2[2H])c([2H])c1[2H]] | 81 |
| 1-189 | 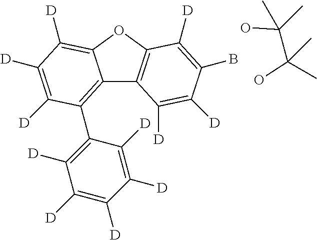 | 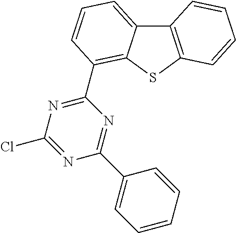 [SMILES: Clc1nc(-c2ccccc2)nc(-c2cccc3c2sc2ccccc23)n1] | 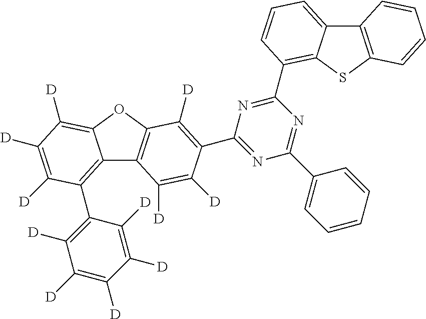 [SMILES: [2H]c1c([2H])c([2H])c(-c2c([2H])c([2H])c([2H])c3oc4c([2H])c(-c5nc(-c6ccccc6)nc(-c6cccc7c6sc6ccccc67)n5)c([2H])c([2H])c4c23)c([2H])c1[2H]] | 82 |
| 1-191 | 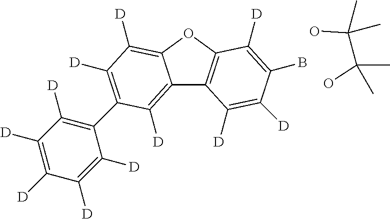 | 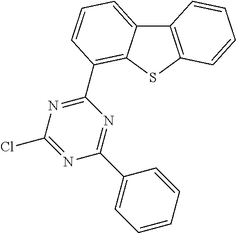 [SMILES: Clc1nc(-c2ccccc2)nc(-c2cccc3c2sc2ccccc23)n1] | 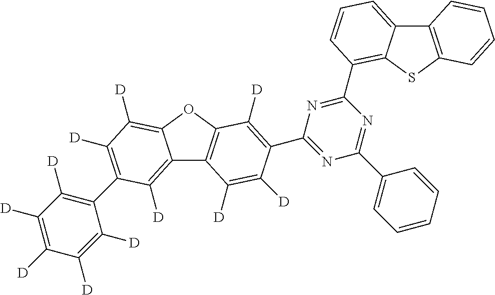 [SMILES: [2H]c1c([2H])c([2H])c(-c2c([2H])c([2H])c3oc4c([2H])c(-c5nc(-c6ccccc6)nc(-c6cccc7c6sc6ccccc67)n5)c([2H])c([2H])c4c3c2[2H])c([2H])c1[2H]] | 83 |
| 1-193 | 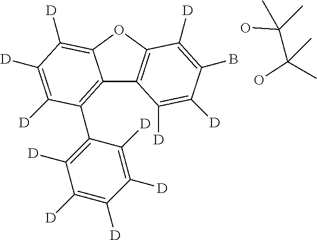 | 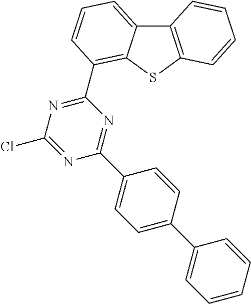 [SMILES: Clc1nc(-c2ccc(-c3ccccc3)cc2)nc(-c2cccc3c2sc2ccccc23)n1] | 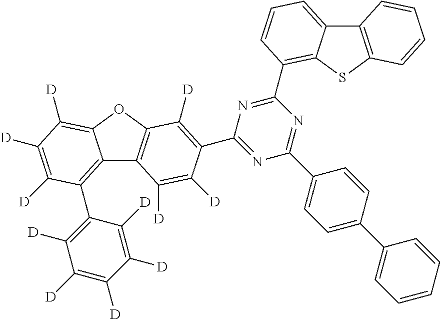 [SMILES: [2H]c1c([2H])c([2H])c(-c2c([2H])c([2H])c([2H])c3oc4c([2H])c(-c5nc(-c6ccc(-c7ccccc7)cc6)nc(-c6cccc7c6sc6ccccc67)n5)c([2H])c([2H])c4c23)c([2H])c1[2H]] | 88 |
| 1-195 | 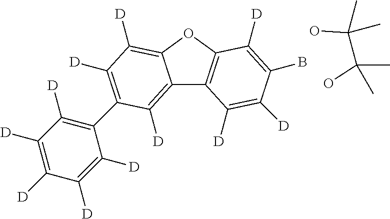 | 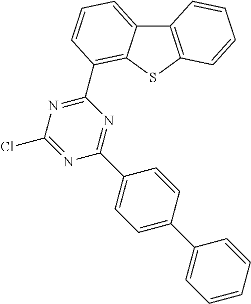 [SMILES: Clc1nc(-c2ccc(-c3ccccc3)cc2)nc(-c2cccc3c2sc2ccccc23)n1] | 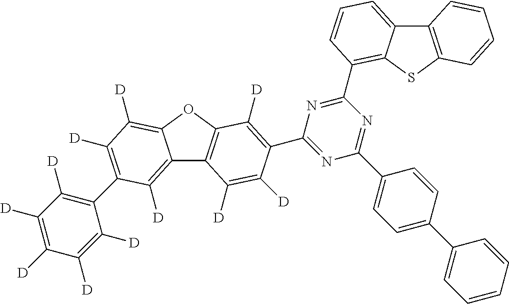 [SMILES: [2H]c1c([2H])c([2H])c(-c2c([2H])c([2H])c3oc4c([2H])c(-c5nc(-c6ccc(-c7ccccc7)cc6)nc(-c6cccc7c6sc6ccccc67)n5)c([2H])c([2H])c4c3c2[2H])c([2H])c1[2H]] | 85 |
| 1-196 | 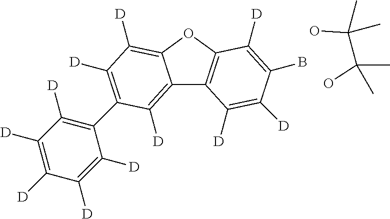 | 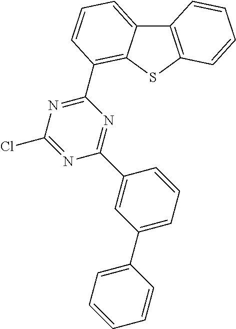 [SMILES: Clc1nc(-c2cccc(-c3ccccc3)c2)nc(-c2cccc3c2sc2ccccc23)n1] | 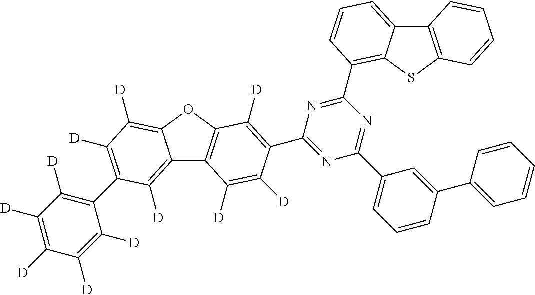 [SMILES: [2H]c1c([2H])c([2H])c(-c2c([2H])c([2H])c3oc4c([2H])c(-c5nc(-c6cccc(-c7ccccc7)c6)nc(-c6cccc7c6sc6ccccc67)n5)c([2H])c([2H])c4c3c2[2H])c([2H])c1[2H]] | 83 |
| 1-197 | 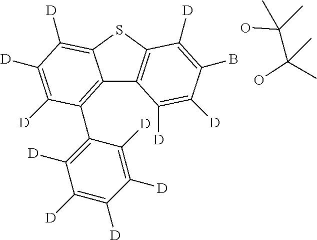 | 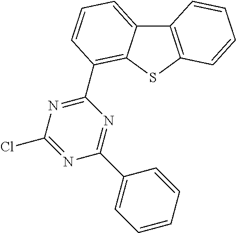 [SMILES: Clc1nc(-c2ccccc2)nc(-c2cccc3c2sc2ccccc23)n1] | 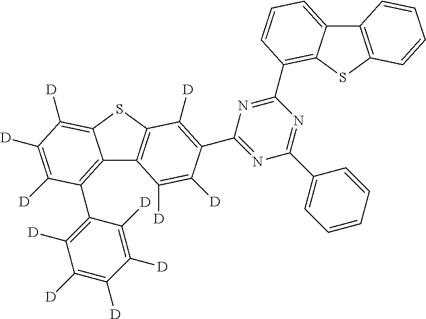 [SMILES: [2H]c1c([2H])c([2H])c(-c2c([2H])c([2H])c([2H])c3sc4c([2H])c(-c5nc(-c6ccccc6)nc(-c6cccc7c6sc6ccccc67)n5)c([2H])c([2H])c4c23)c([2H])c1[2H]] | 84 |
| 1-199 | 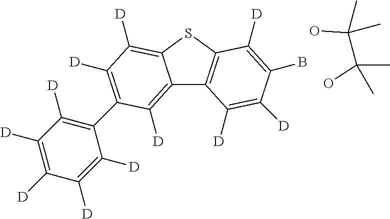 | 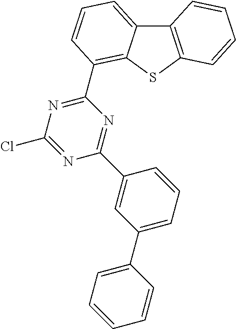 [SMILES: Clc1nc(-c2cccc(-c3ccccc3)c2)nc(-c2cccc3c2sc2ccccc23)n1] | 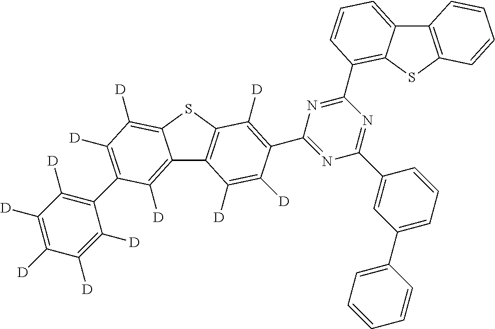 [SMILES: [2H]c1c([2H])c([2H])c(-c2c([2H])c([2H])c3sc4c([2H])c(-c5nc(-c6cccc(-c7ccccc7)c6)nc(-c6cccc7c6sc6ccccc67)n5)c([2H])c([2H])c4c3c2[2H])c([2H])c1[2H]] | 83 |
| 1-201 | 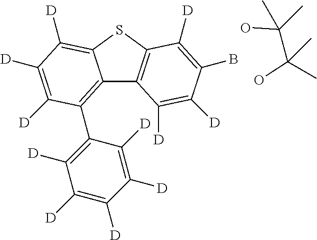 | 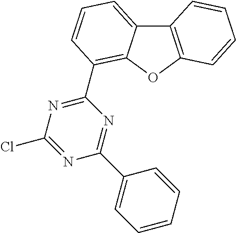 [SMILES: Clc1nc(-c2ccccc2)nc(-c2cccc3c2oc2ccccc23)n1] | 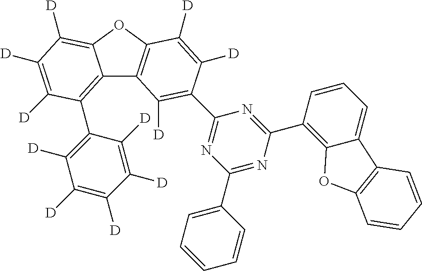 [SMILES: [2H]c1c([2H])c([2H])c(-c2c([2H])c([2H])c([2H])c3oc4c([2H])c([2H])c(-c5nc(-c6ccccc6)nc(-c6cccc7c6oc6ccccc67)n5)c([2H])c4c23)c([2H])c1[2H]] | 80 |
| 1-203 | 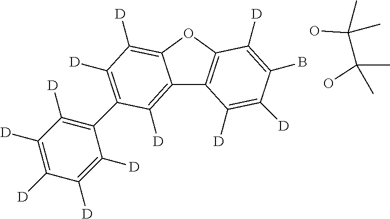 | 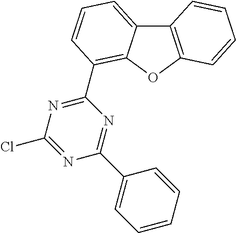 [SMILES: Clc1nc(-c2ccccc2)nc(-c2cccc3c2oc2ccccc23)n1] | 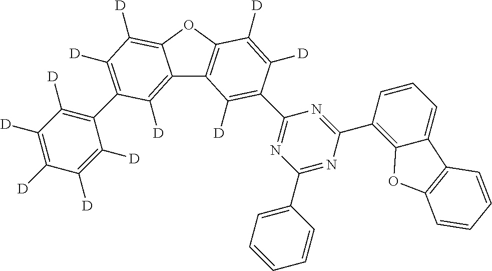 [SMILES: [2H]c1c([2H])c([2H])c(-c2c([2H])c([2H])c3oc4c([2H])c([2H])c(-c5nc(-c6ccccc6)nc(-c6cccc7c6oc6ccccc67)n5)c([2H])c4c3c2[2H])c([2H])c1[2H]] | 88 |
| 1-205 | 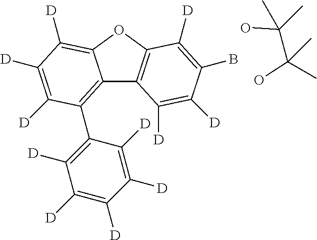 | 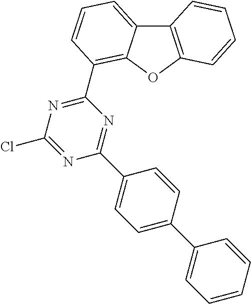 [SMILES: Clc1nc(-c2ccc(-c3ccccc3)cc2)nc(-c2cccc3c2oc2ccccc23)n1] | 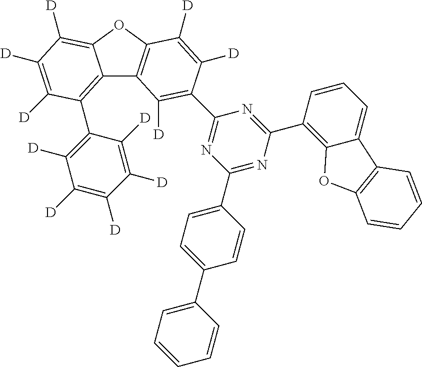 [SMILES: [2H]c1c([2H])c([2H])c(-c2c([2H])c([2H])c([2H])c3oc4c([2H])c([2H])c(-c5nc(-c6ccc(-c7ccccc7)cc6)nc(-c6cccc7c6oc6ccccc67)n5)c([2H])c4c23)c([2H])c1[2H]] | 86 |
| 1-207 | 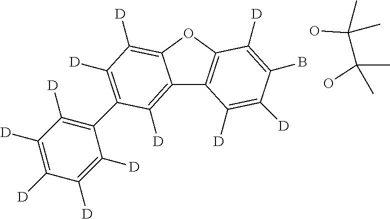 | 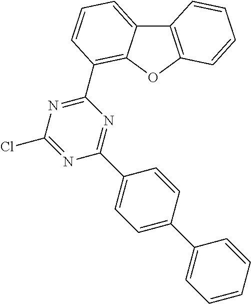 [SMILES: Clc1nc(-c2ccc(-c3ccccc3)cc2)nc(-c2cccc3c2oc2ccccc23)n1] | 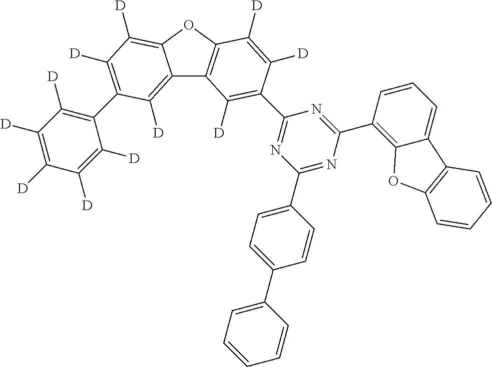 [SMILES: [2H]c1c([2H])c([2H])c(-c2c([2H])c([2H])c3oc4c([2H])c([2H])c(-c5nc(-c6ccc(-c7ccccc7)cc6)nc(-c6cccc7c6oc6ccccc67)n5)c([2H])c4c3c2[2H])c([2H])c1[2H]] | 85 |
| 1-208 | 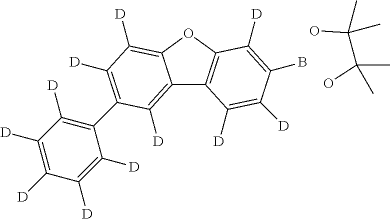 | 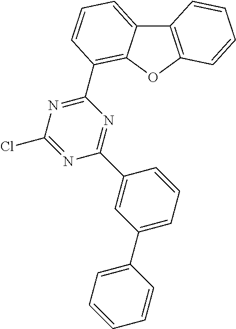 [SMILES: Clc1nc(-c2cccc(-c3ccccc3)c2)nc(-c2cccc3c2oc2ccccc23)n1] | 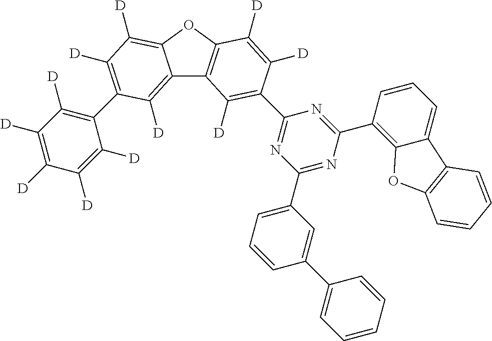 [SMILES: [2H]c1c([2H])c([2H])c(-c2c([2H])c([2H])c3oc4c([2H])c([2H])c(-c5nc(-c6cccc(-c7ccccc7)c6)nc(-c6cccc7c6oc6ccccc67)n5)c([2H])c4c3c2[2H])c([2H])c1[2H]] | 83 |
| 1-209 | 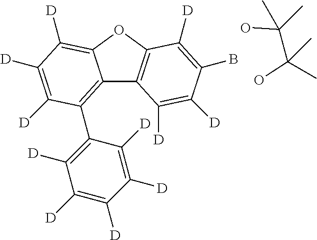 | 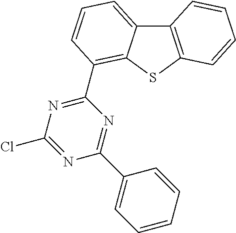 [SMILES: Clc1nc(-c2ccccc2)nc(-c2cccc3c2sc2ccccc23)n1] | 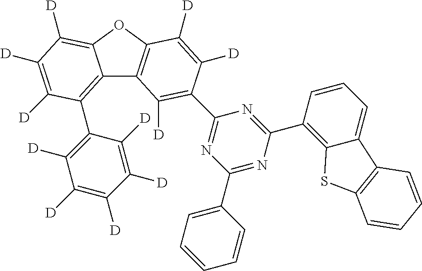 [SMILES: [2H]c1c([2H])c([2H])c(-c2c([2H])c([2H])c([2H])c3oc4c([2H])c([2H])c(-c5nc(-c6ccccc6)nc(-c6cccc7c6sc6ccccc67)n5)c([2H])c4c23)c([2H])c1[2H]] | 84 |
| 1-211 | 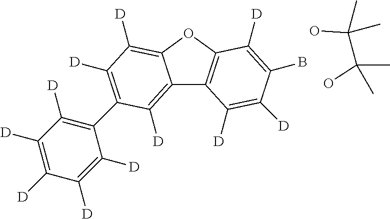 | 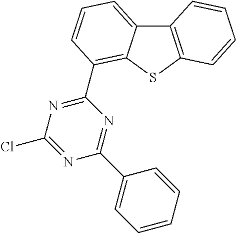 [SMILES: Clc1nc(-c2ccccc2)nc(-c2cccc3c2sc2ccccc23)n1] | 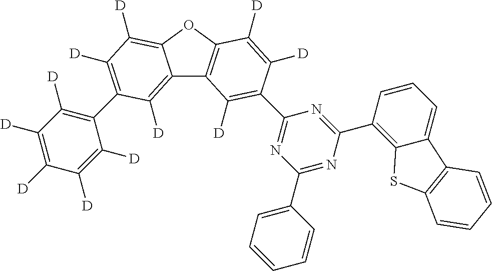 [SMILES: [2H]c1c([2H])c([2H])c(-c2c([2H])c([2H])c3oc4c([2H])c([2H])c(-c5nc(-c6ccccc6)nc(-c6cccc7c6sc6ccccc67)n5)c([2H])c4c3c2[2H])c([2H])c1[2H]] | 88 |
| 1-213 | 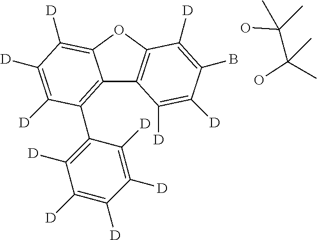 | 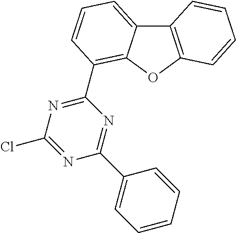 [SMILES: Clc1nc(-c2ccccc2)nc(-c2cccc3c2oc2ccccc23)n1] | 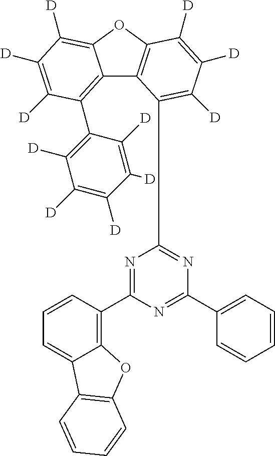 [SMILES: [2H]c1c([2H])c([2H])c(-c2c([2H])c([2H])c([2H])c3oc4c([2H])c([2H])c([2H])c(-c5nc(-c6ccccc6)nc(-c6cccc7c6oc6ccccc67)n5)c4c23)c([2H])c1[2H]] | 86 |
| 1-215 | 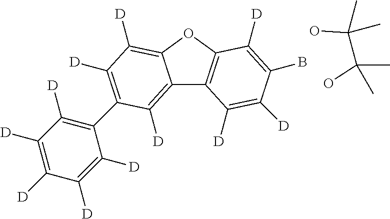 | 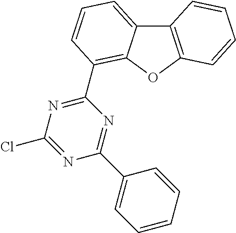 [SMILES: Clc1nc(-c2ccccc2)nc(-c2cccc3c2oc2ccccc23)n1] | 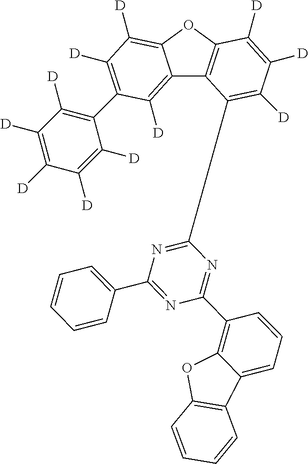 [SMILES: [2H]c1c([2H])c([2H])c(-c2c([2H])c([2H])c3oc4c([2H])c([2H])c([2H])c(-c5nc(-c6ccccc6)nc(-c6cccc7c6oc6ccccc67)n5)c4c3c2[2H])c([2H])c1[2H]] | 84 |
| 1-217 | 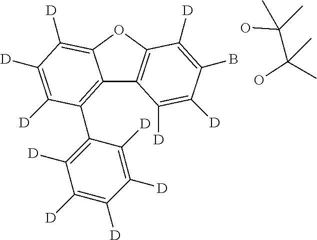 | 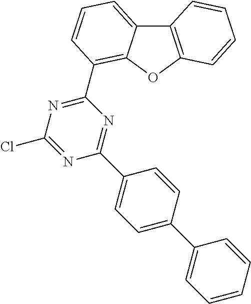 [SMILES: Clc1nc(-c2ccc(-c3ccccc3)cc2)nc(-c2cccc3c2oc2ccccc23)n1] | 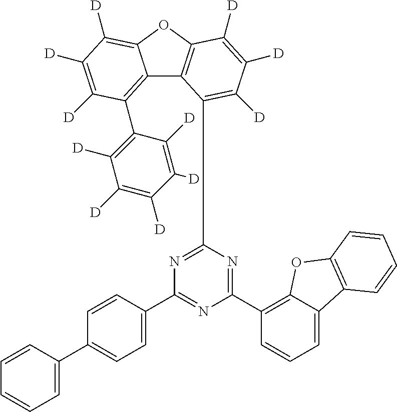 [SMILES: [2H]c1c([2H])c([2H])c(-c2c([2H])c([2H])c([2H])c3oc4c([2H])c([2H])c([2H])c(-c5nc(-c6ccc(-c7ccccc7)cc6)nc(-c6cccc7c6oc6ccccc67)n5)c4c23)c([2H])c1[2H]] | 85 |
| 1-219 | 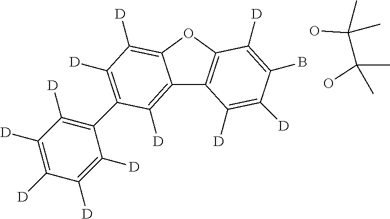 | 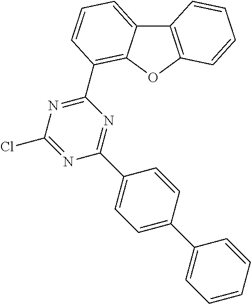 [SMILES: Clc1nc(-c2ccc(-c3ccccc3)cc2)nc(-c2cccc3c2oc2ccccc23)n1] | 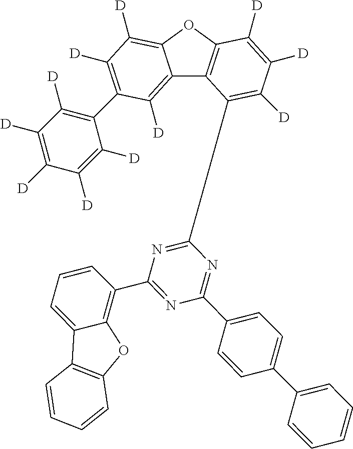 [SMILES: [2H]c1c([2H])c([2H])c(-c2c([2H])c([2H])c3oc4c([2H])c([2H])c([2H])c(-c5nc(-c6ccc(-c7ccccc7)cc6)nc(-c6cccc7c6oc6ccccc67)n5)c4c3c2[2H])c([2H])c1[2H]] | 83 |
| 1-220 | 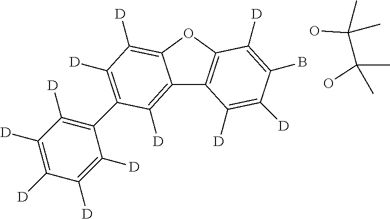 | 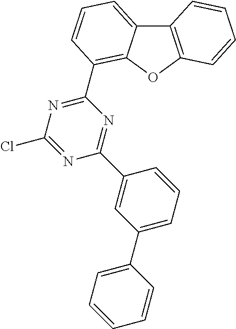 [SMILES: Clc1nc(-c2cccc(-c3ccccc3)c2)nc(-c2cccc3c2oc2ccccc23)n1] | 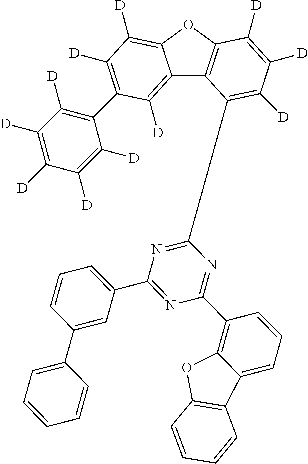 [SMILES: [2H]c1c([2H])c([2H])c(-c2c([2H])c([2H])c3oc4c([2H])c([2H])c([2H])c(-c5nc(-c6cccc(-c7ccccc7)c6)nc(-c6cccc7c6oc6ccccc67)n5)c4c3c2[2H])c([2H])c1[2H]] | 83 |

### <Preparation Example 4> Preparation of Compound 1-182(H)

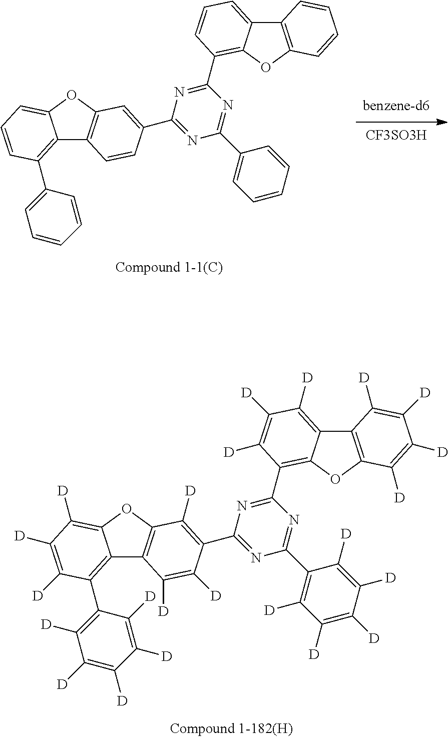 [SMILES: c1ccc(-c2nc(-c3ccc4c(c3)oc3cccc(-c5ccccc5)c34)nc(-c3cccc4c3oc3ccccc34)n2)cc1 ; "CF3SO3H" ; [2H]c1c([2H])c([2H])c(-c2nc(-c3c([2H])c([2H])c4c(oc5c([2H])c([2H])c([2H])c(-c6c([2H])c([2H])c([2H])c([2H])c6[2H])c54)c3[2H])nc(-c3c([2H])c([2H])c([2H])c4c3oc3c([2H])c([2H])c([2H])c([2H])c34)n2)c([2H])c1[2H]]

Compound 1-1(C) (10 g, 1 eq.), benzene-d6 (500 g, 287.93 eq.), and CF₃SO₃H (245 g, 75 eq.) were put into a reaction flask, and then stirred at 50° C. for 1 hour. When the reaction was completed, H₂O was added thereto for neutralization, then the resulting product was extracted with MC and H₂O, and then column-purified to obtain Compound 1-182(H). (6.8 g, 67%)

The following Compound (H) was synthesized in the same manner as in Preparation Example 4 using compounds in the following Table 4 instead of Compound 1-1(C) in Preparation Example 4.

TABLE 4

| Com- | | | Yield |
|---|---|---|---|
| pound | Compound (C) | Compound (H) | (%) |
| 1-184 | 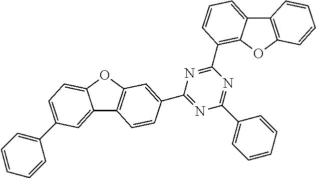 [SMILES: c1ccc(-c2ccc3oc4cc(-c5nc(-c6ccccc6)nc(-c6cccc7c6oc6ccccc67)n5)ccc4c3c2)cc1] | 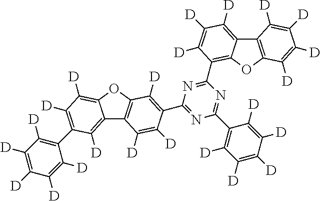 [SMILES: [2H]c1c([2H])c([2H])c(-c2nc(-c3c([2H])c([2H])c4c(oc5c([2H])c([2H])c(-c6c([2H])c([2H])c([2H])c([2H])c6[2H])c([2H])c54)c3[2H])nc(-c3c([2H])c([2H])c([2H])c4c3oc3c([2H])c([2H])c([2H])c([2H])c34)n2)c([2H])c1[2H]] | 78 |
| 1-186 | 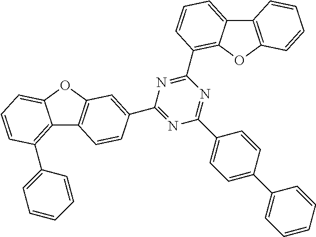 [SMILES: c1ccc(-c2ccc(-c3nc(-c4ccc5c(c4)oc4cccc(-c6ccccc6)c45)nc(-c4cccc5c4oc4ccccc45)n3)cc2)cc1] | 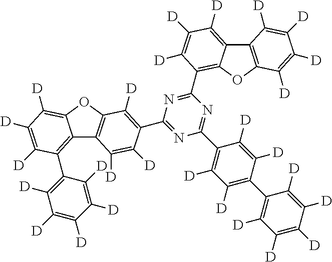 [SMILES: [2H]c1c([2H])c([2H])c(-c2c([2H])c([2H])c(-c3nc(-c4c([2H])c([2H])c5c(oc6c([2H])c([2H])c([2H])c(-c7c([2H])c([2H])c([2H])c([2H])c7[2H])c65)c4[2H])nc(-c4c([2H])c([2H])c([2H])c5c4oc4c([2H])c([2H])c([2H])c([2H])c45)n3)c([2H])c2[2H])c([2H])c1[2H]] | 77 |
| 1-190 | 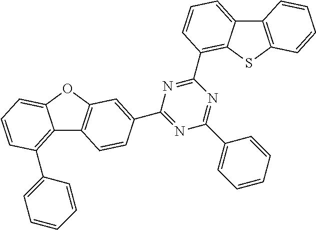 [SMILES: c1ccc(-c2nc(-c3ccc4c(c3)oc3cccc(-c5ccccc5)c34)nc(-c3cccc4c3sc3ccccc34)n2)cc1] | 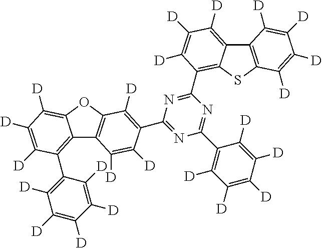 [SMILES: [2H]c1c([2H])c([2H])c(-c2nc(-c3c([2H])c([2H])c4c(oc5c([2H])c([2H])c([2H])c(-c6c([2H])c([2H])c([2H])c([2H])c6[2H])c54)c3[2H])nc(-c3c([2H])c([2H])c([2H])c4c3sc3c([2H])c([2H])c([2H])c([2H])c34)n2)c([2H])c1[2H]] | 68 |
| 1-192 | 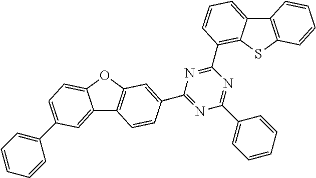 [SMILES: c1ccc(-c2ccc3oc4cc(-c5nc(-c6ccccc6)nc(-c6cccc7c6sc6ccccc67)n5)ccc4c3c2)cc1] | 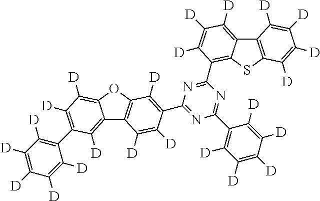 [SMILES: [2H]c1c([2H])c([2H])c(-c2nc(-c3c([2H])c([2H])c4c(oc5c([2H])c([2H])c(-c6c([2H])c([2H])c([2H])c([2H])c6[2H])c([2H])c54)c3[2H])nc(-c3c([2H])c([2H])c([2H])c4c3sc3c([2H])c([2H])c([2H])c([2H])c34)n2)c([2H])c1[2H]] | 78 |
| 1-194 | 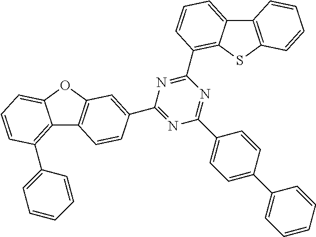 [SMILES: c1ccc(-c2ccc(-c3nc(-c4ccc5c(c4)oc4cccc(-c6ccccc6)c45)nc(-c4cccc5c4sc4ccccc45)n3)cc2)cc1] | 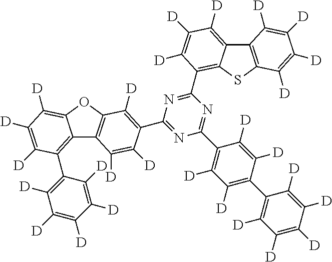 [SMILES: [2H]c1c([2H])c([2H])c(-c2c([2H])c([2H])c(-c3nc(-c4c([2H])c([2H])c5c(oc6c([2H])c([2H])c([2H])c(-c7c([2H])c([2H])c([2H])c([2H])c7[2H])c65)c4[2H])nc(-c4c([2H])c([2H])c([2H])c5c4sc4c([2H])c([2H])c([2H])c([2H])c45)n3)c([2H])c2[2H])c([2H])c1[2H]] | 75 |
| 1-198 | 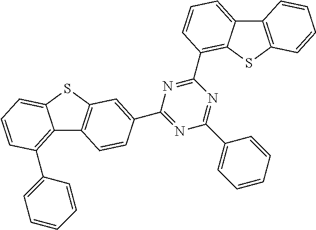 [SMILES: c1ccc(-c2nc(-c3ccc4c(c3)sc3cccc(-c5ccccc5)c34)nc(-c3cccc4c3sc3ccccc34)n2)cc1] | 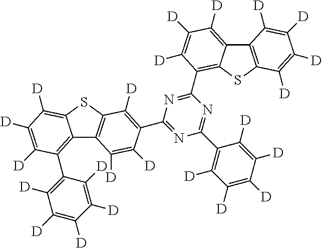 [SMILES: [2H]c1c([2H])c([2H])c(-c2nc(-c3c([2H])c([2H])c4c(sc5c([2H])c([2H])c([2H])c(-c6c([2H])c([2H])c([2H])c([2H])c6[2H])c54)c3[2H])nc(-c3c([2H])c([2H])c([2H])c4c3sc3c([2H])c([2H])c([2H])c([2H])c34)n2)c([2H])c1[2H]] | 72 |
| 1-200 | 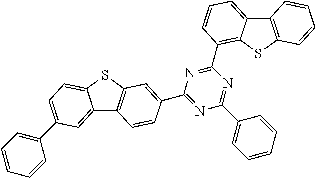 [SMILES: c1ccc(-c2ccc3sc4cc(-c5nc(-c6ccccc6)nc(-c6cccc7c6sc6ccccc67)n5)ccc4c3c2)cc1] | 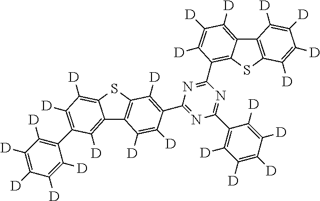 [SMILES: [2H]c1c([2H])c([2H])c(-c2nc(-c3c([2H])c([2H])c4c(sc5c([2H])c([2H])c(-c6c([2H])c([2H])c([2H])c([2H])c6[2H])c([2H])c54)c3[2H])nc(-c3c([2H])c([2H])c([2H])c4c3sc3c([2H])c([2H])c([2H])c([2H])c34)n2)c([2H])c1[2H]] | 73 |
| 1-202 | 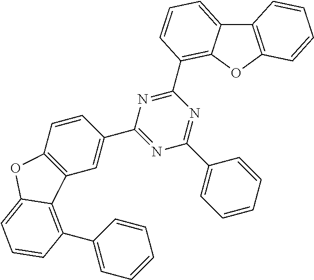 [SMILES: c1ccc(-c2nc(-c3ccc4oc5cccc(-c6ccccc6)c5c4c3)nc(-c3cccc4c3oc3ccccc34)n2)cc1] | 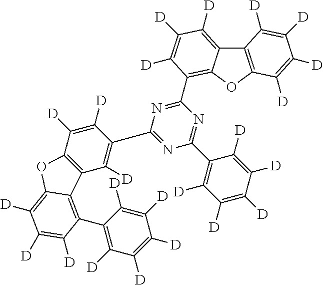 [SMILES: [2H]c1c([2H])c([2H])c(-c2nc(-c3c([2H])c([2H])c4oc5c([2H])c([2H])c([2H])c(-c6c([2H])c([2H])c([2H])c([2H])c6[2H])c5c4c3[2H])nc(-c3c([2H])c([2H])c([2H])c4c3oc3c([2H])c([2H])c([2H])c([2H])c34)n2)c([2H])c1[2H]] | 72 |
| 1-204 | 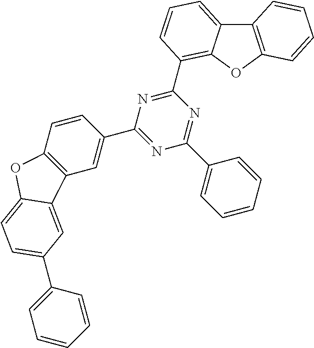 [SMILES: c1ccc(-c2ccc3oc4ccc(-c5nc(-c6ccccc6)nc(-c6cccc7c6oc6ccccc67)n5)cc4c3c2)cc1] | 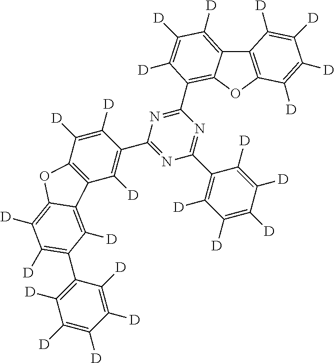 [SMILES: [2H]c1c([2H])c([2H])c(-c2nc(-c3c([2H])c([2H])c4oc5c([2H])c([2H])c(-c6c([2H])c([2H])c([2H])c([2H])c6[2H])c([2H])c5c4c3[2H])nc(-c3c([2H])c([2H])c([2H])c4c3oc3c([2H])c([2H])c([2H])c([2H])c34)n2)c([2H])c1[2H]] | 77 |
| 1-206 | 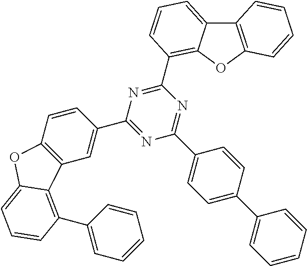 [SMILES: c1ccc(-c2ccc(-c3nc(-c4ccc5oc6cccc(-c7ccccc7)c6c5c4)nc(-c4cccc5c4oc4ccccc45)n3)cc2)cc1] | 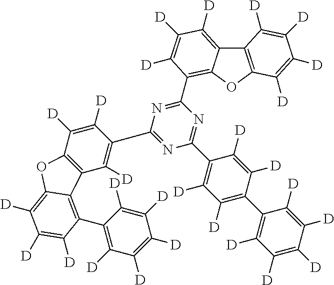 [SMILES: [2H]c1c([2H])c([2H])c(-c2c([2H])c([2H])c(-c3nc(-c4c([2H])c([2H])c5oc6c([2H])c([2H])c([2H])c(-c7c([2H])c([2H])c([2H])c([2H])c7[2H])c6c5c4[2H])nc(-c4c([2H])c([2H])c([2H])c5c4oc4c([2H])c([2H])c([2H])c([2H])c45)n3)c([2H])c2[2H])c([2H])c1[2H]] | 78 |
| 1-210 | 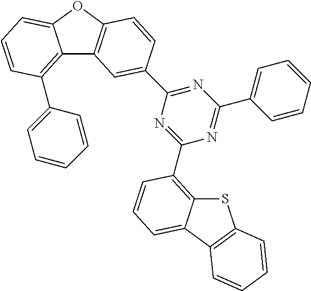 [SMILES: c1ccc(-c2nc(-c3ccc4oc5cccc(-c6ccccc6)c5c4c3)nc(-c3cccc4c3sc3ccccc34)n2)cc1] | 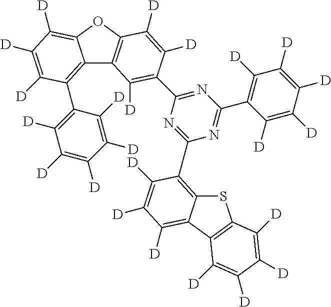 [SMILES: [2H]c1c([2H])c([2H])c(-c2nc(-c3c([2H])c([2H])c4oc5c([2H])c([2H])c([2H])c(-c6c([2H])c([2H])c([2H])c([2H])c6[2H])c5c4c3[2H])nc(-c3c([2H])c([2H])c([2H])c4c3sc3c([2H])c([2H])c([2H])c([2H])c34)n2)c([2H])c1[2H]] | 76 |
| 1-212 | 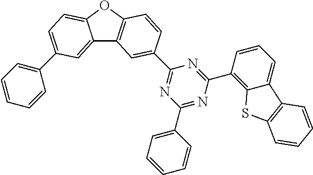 [SMILES: c1ccc(-c2ccc3oc4ccc(-c5nc(-c6ccccc6)nc(-c6cccc7c6sc6ccccc67)n5)cc4c3c2)cc1] | 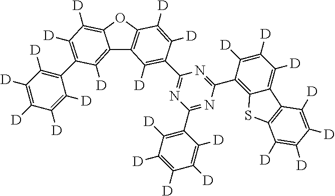 [SMILES: [2H]c1c([2H])c([2H])c(-c2nc(-c3c([2H])c([2H])c4oc5c([2H])c([2H])c(-c6c([2H])c([2H])c([2H])c([2H])c6[2H])c([2H])c5c4c3[2H])nc(-c3c([2H])c([2H])c([2H])c4c3sc3c([2H])c([2H])c([2H])c([2H])c34)n2)c([2H])c1[2H]] | 75 |
| 1-214 | 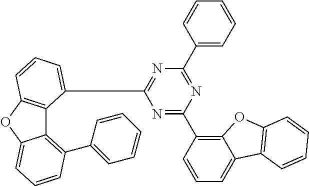 [SMILES: c1ccc(-c2nc(-c3cccc4c3oc3ccccc34)nc(-c3cccc4oc5cccc(-c6ccccc6)c5c34)n2)cc1] | 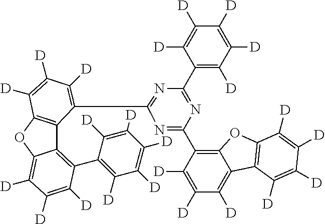 [SMILES: [2H]c1c([2H])c([2H])c(-c2nc(-c3c([2H])c([2H])c([2H])c4c3oc3c([2H])c([2H])c([2H])c([2H])c34)nc(-c3c([2H])c([2H])c([2H])c4oc5c([2H])c([2H])c([2H])c(-c6c([2H])c([2H])c([2H])c([2H])c6[2H])c5c34)n2)c([2H])c1[2H]] | 75 |
| 1-216 | 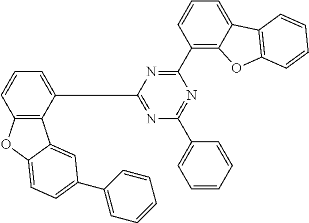 [SMILES: c1ccc(-c2ccc3oc4cccc(-c5nc(-c6ccccc6)nc(-c6cccc7c6oc6ccccc67)n5)c4c3c2)cc1] | 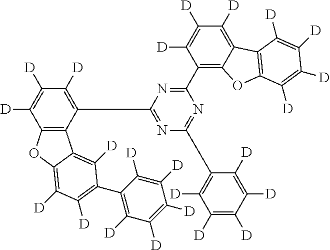 [SMILES: [2H]c1c([2H])c([2H])c(-c2nc(-c3c([2H])c([2H])c([2H])c4c3oc3c([2H])c([2H])c([2H])c([2H])c34)nc(-c3c([2H])c([2H])c([2H])c4oc5c([2H])c([2H])c(-c6c([2H])c([2H])c([2H])c([2H])c6[2H])c([2H])c5c34)n2)c([2H])c1[2H]] | 77 |
| 1-218 | 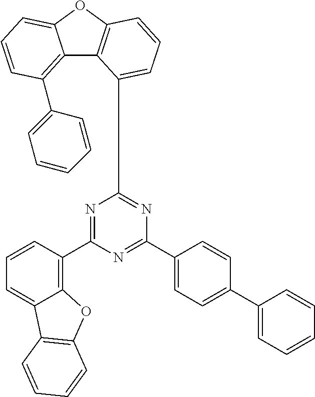 [SMILES: c1ccc(-c2ccc(-c3nc(-c4cccc5c4oc4ccccc45)nc(-c4cccc5oc6cccc(-c7ccccc7)c6c45)n3)cc2)cc1] | 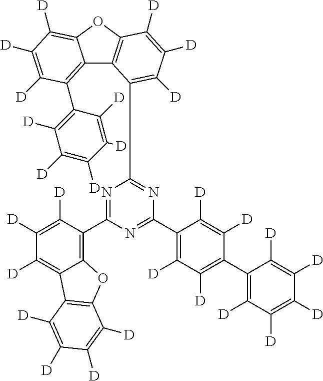 [SMILES: [2H]c1c([2H])c([2H])c(-c2c([2H])c([2H])c(-c3nc(-c4c([2H])c([2H])c([2H])c5c4oc4c([2H])c([2H])c([2H])c([2H])c45)nc(-c4c([2H])c([2H])c([2H])c5oc6c([2H])c([2H])c([2H])c(-c7c([2H])c([2H])c([2H])c([2H])c7[2H])c6c45)n3)c([2H])c2[2H])c([2H])c1[2H]] | 73 |

### <Preparation Example 5> Preparation of Compound 2-1

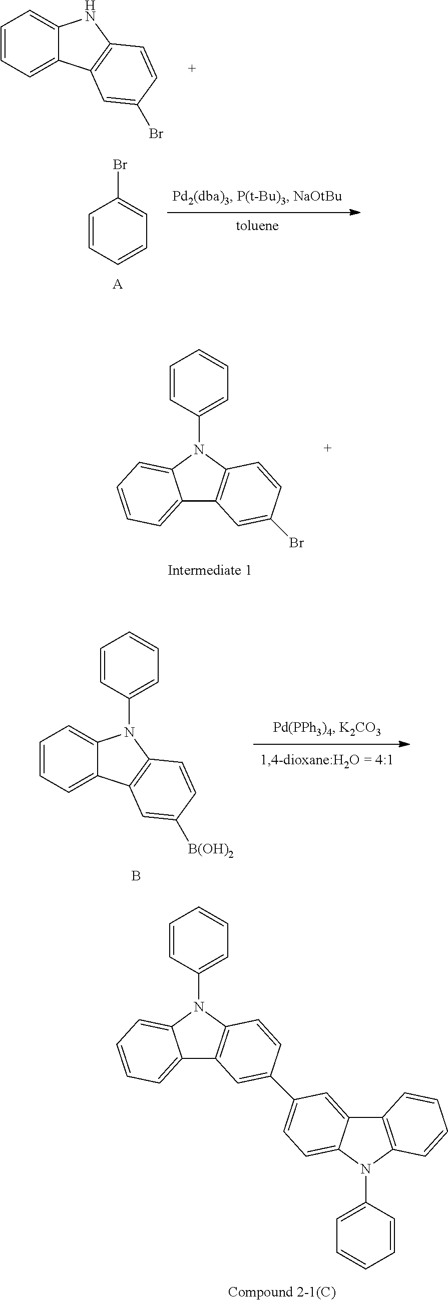

### Synthesis Method of Intermediate 1

After 3-bromo-9H-carbazole (10.0 g, 49.59 mmol), 2-bromobenzene-1-ylium (A) (24.2 g, 148.77 mmol), Pd₂(dba)₃ (2.27 g, 2.48 mmol), P(t-Bu)₃ (2.42 mL, 9.92 mmol), and NaOtBu (9.53 g, 99.18 mmol) were put into a reaction flask, toluene (100 ml) was added thereto, and the resulting mixture was heated at 135° C. for 15 hours. After the reaction was completed, extraction was performed with MC and H₂O, and then column purification was performed to obtain Intermediate 1. (14 g, 98%)

### Synthesis Method of Compound 2-1(C)

After Intermediate 1 (14 g, 43.4 mmol), (9-phenyl-9H-carbazol-3-yl)boronic acid (B) (14.9 g, 52 mmol), Pd(PPh₃)₄ (2.5 g, 2.17 mmol), and K₂CO₃ (17.9 g, 130 mmol) were put into a reaction flask, a mixture of 1,4-dioxane/water (140 ml/35 ml) was heated at 120° C. for 4 hours. After the reaction was completed, the temperature was lowered to room temperature, and then the resulting solid was washed with distilled water and methanol (MeOH) to obtain Compound 2-1 (C). (17 g, 80%)

The following Compound (C) was synthesized in the same manner as in Preparation Example 5 using compounds in the following Table 5 instead of Compounds A and B in Preparation Example 5.

TABLE 5

| Com- | | | | Yield |
|---|---|---|---|---|
| pound | A | B | C | (%) |
| 2-2 | 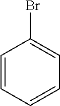 | 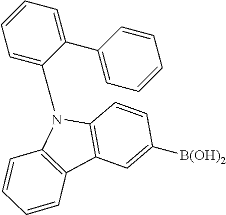 | 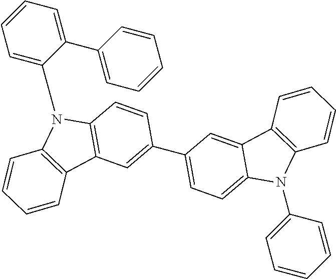 | 82 |
| 2-3 | 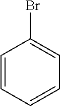 | 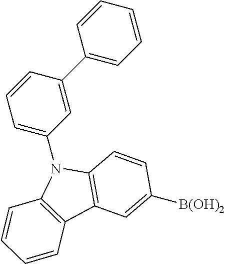 | 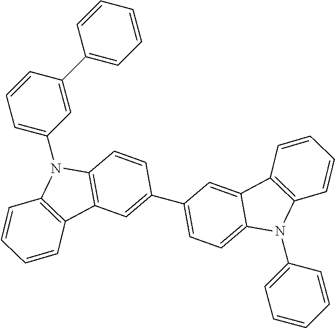 | 80 |
| 2-4 | 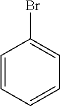 | 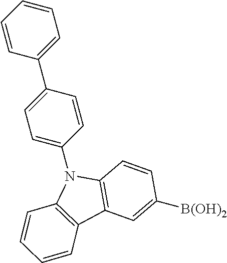 | 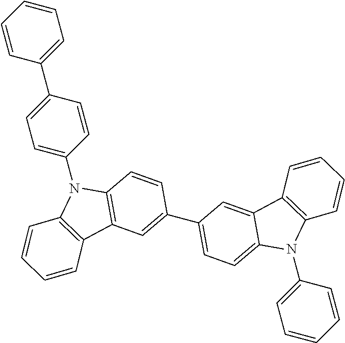 | 83 |
| 2-5 | 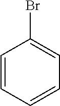 | 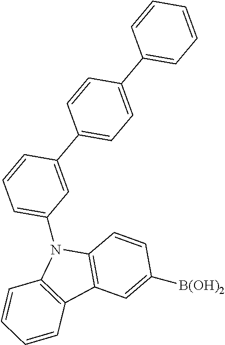 | 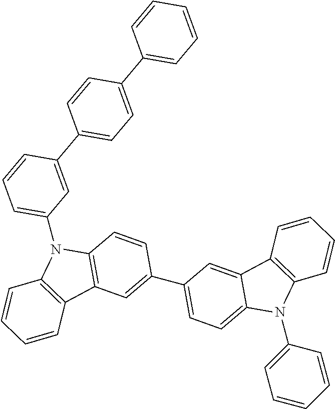 | 88 |
| 2-6 | 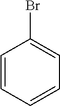 | 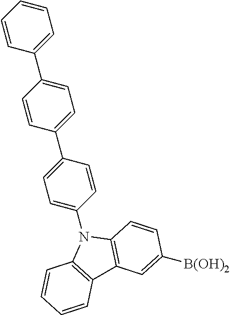 | 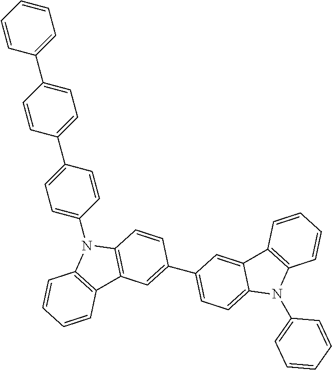 | 86 |
| 2-7 | 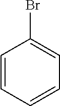 | 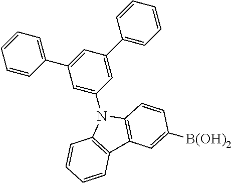 | 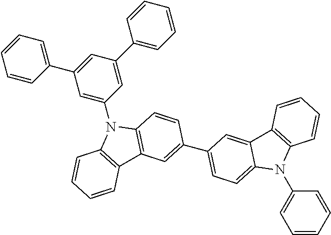 | 80 |
| 2-11 | 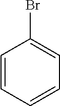 | 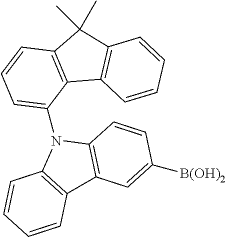 | 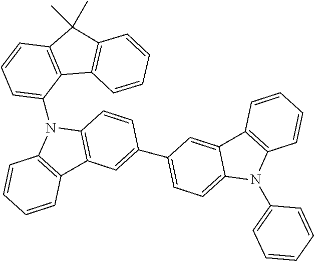 | 81 |
| 2-16 | 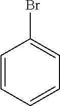 | 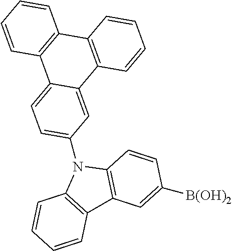 | 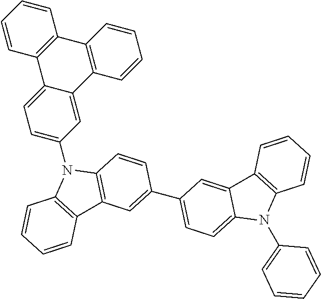 | 80 |
| 2-19 | 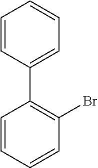 | 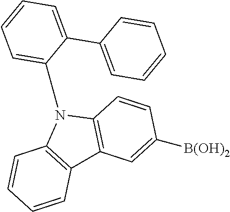 | 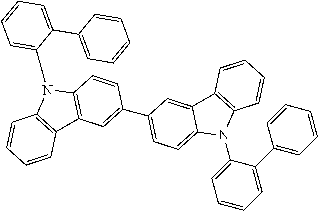 | 83 |
| 2-20 | 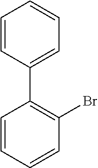 | 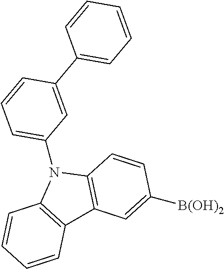 | 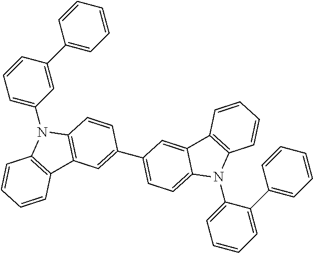 | 81 |
| 2-21 | 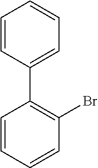 | 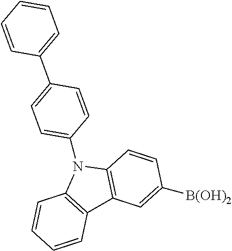 | 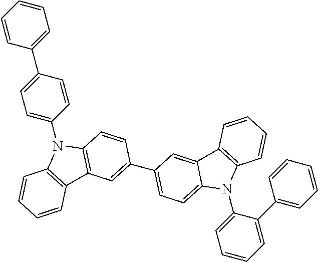 | 80 |
| 2-22 | 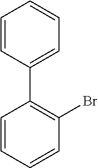 | 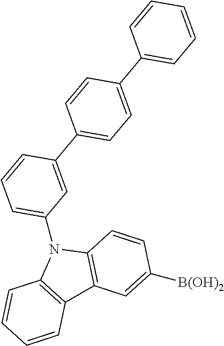 | 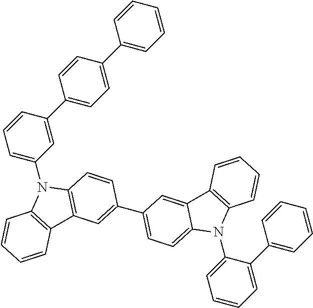 | 79 |
| 2-23 | 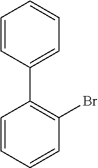 | 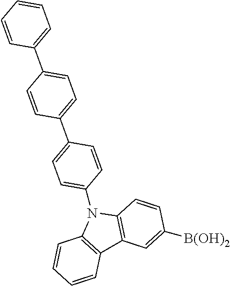 | 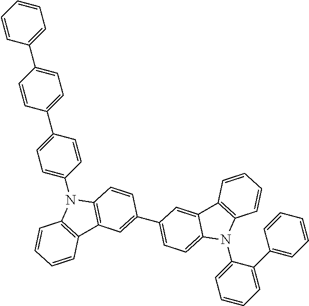 | 82 |
| 2-26 | 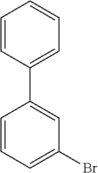 | 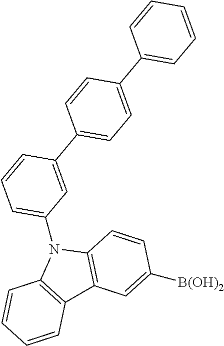 | 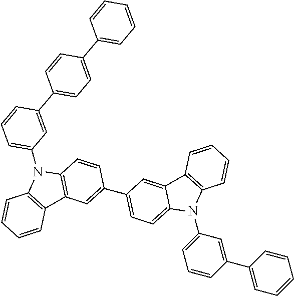 | 81 |
| 2-27 | 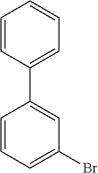 | 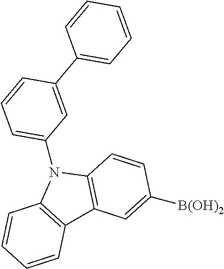 | 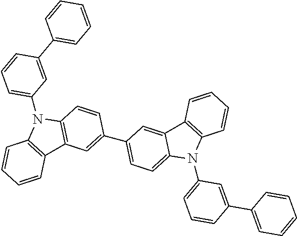 | 85 |
| 2-28 | 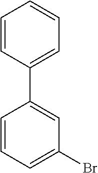 | 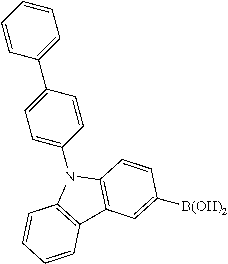 | 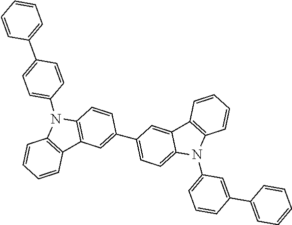 | 81 |
| 2-29 | 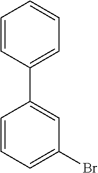 | 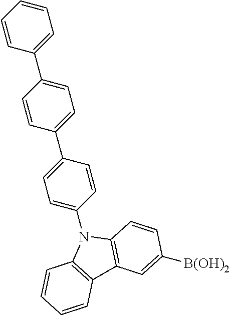 | 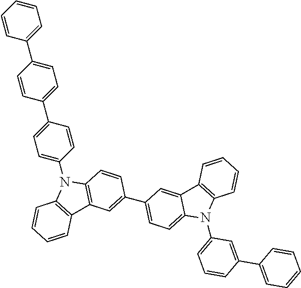 | 80 |
| 2-30 | 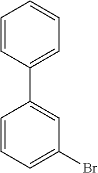 | 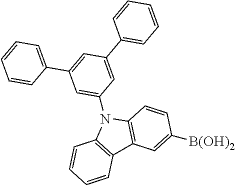 | 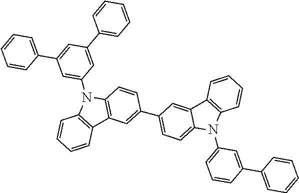 | 79 |
| 2-32 | 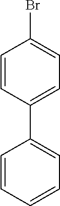 | 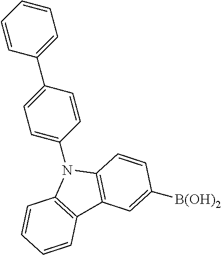 | 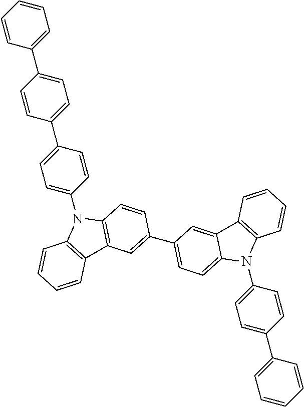 | 81 |
| 2-33 | 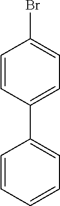 | 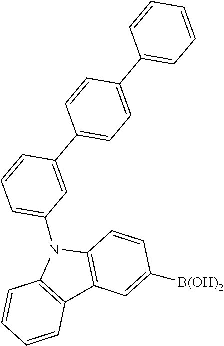 | 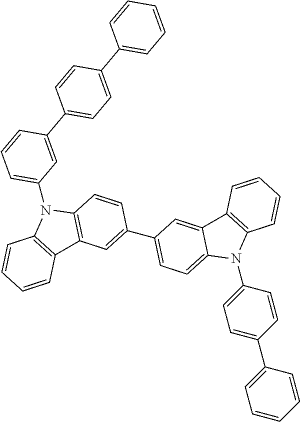 | 81 |
| 2-34 | 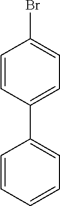 | 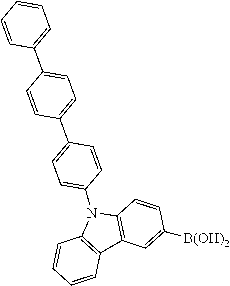 | 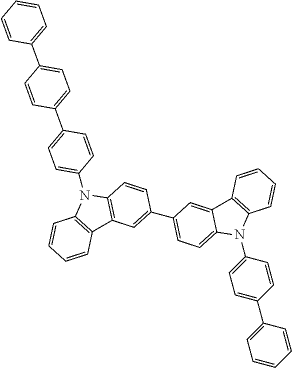 | 82 |
| 2-38 | 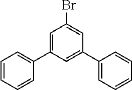 | 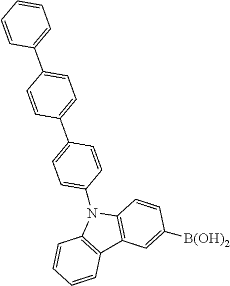 | 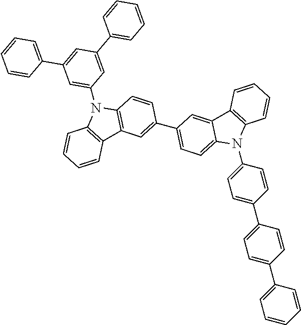 | 82 |
| 2-40 | 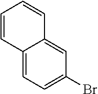 | 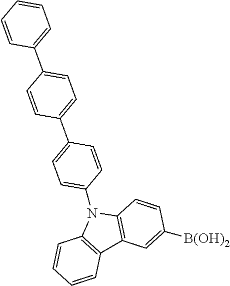 | 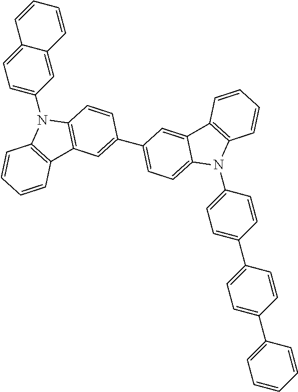 | 81 |
| 2-41 | 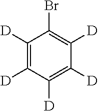 | 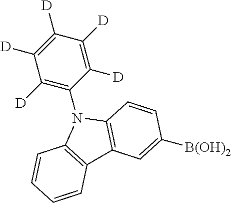 | 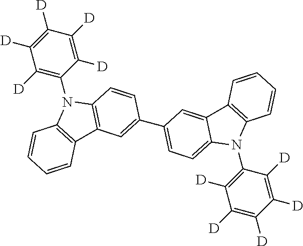 | 80 |
| 2-42 | 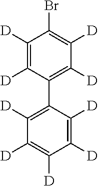 | 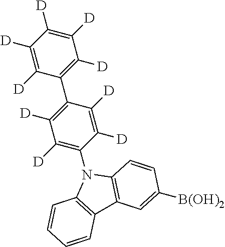 | 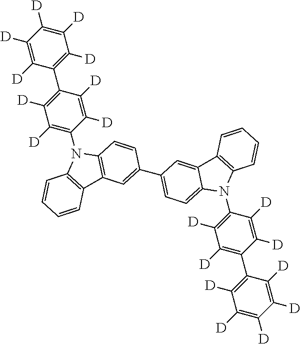 | 79 |
| 2-43 | 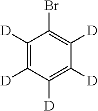 | 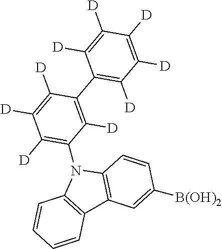 | 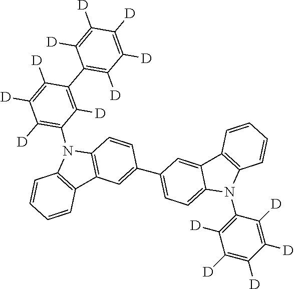 | 78 |
| 2-45 | 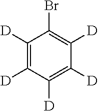 | 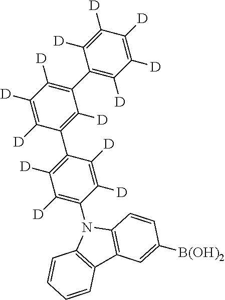 | 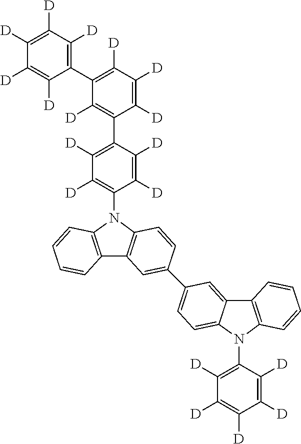 | 79 |
| 2-46 | 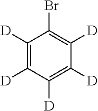 | 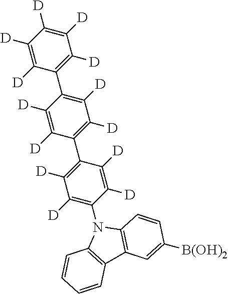 | 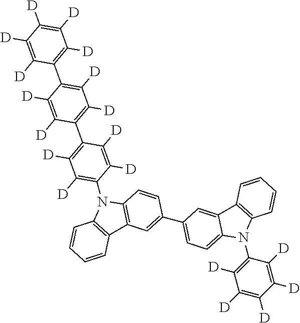 | 80 |
| 2-48 | 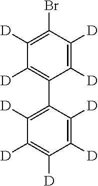 | 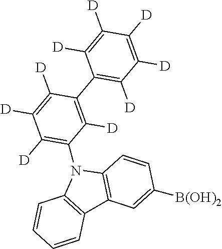 | 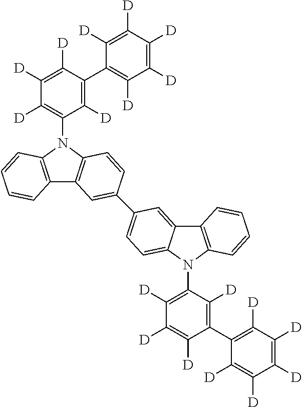 | 81 |
| 2-49 | 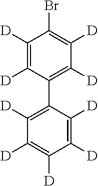 | 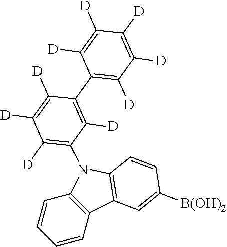 | 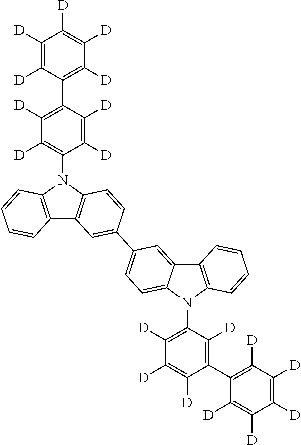 | 83 |
| 2-50 | 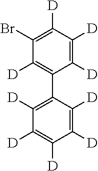 | 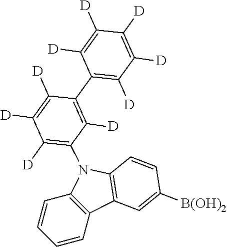 | 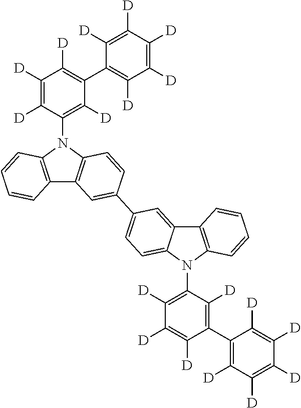 | 82 |
| 2-51 | 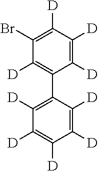 | 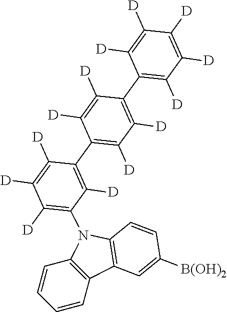 | 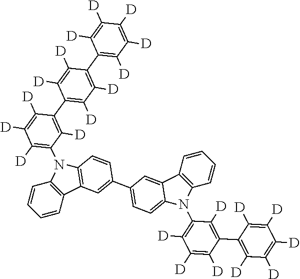 | 84 |
| 2-52 | 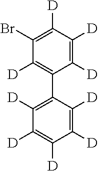 | 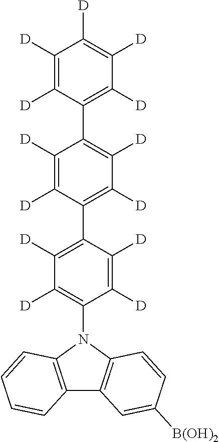 | 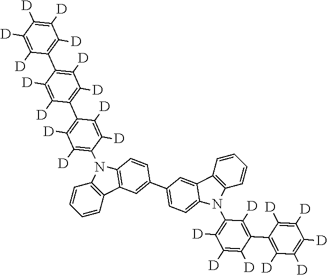 | 80 |
| 2-55 | 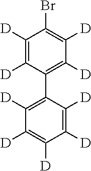 | 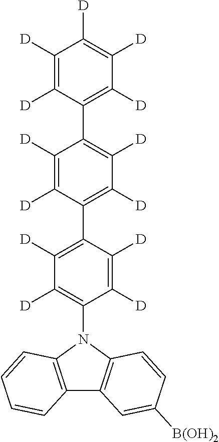 | 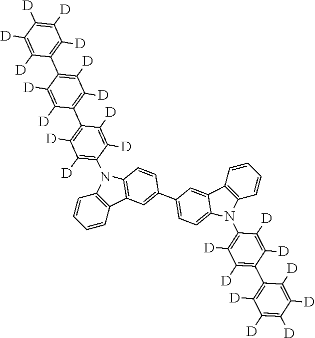 | 81 |
| 2-57 | 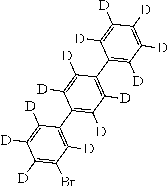 | 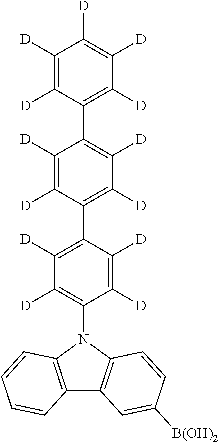 | 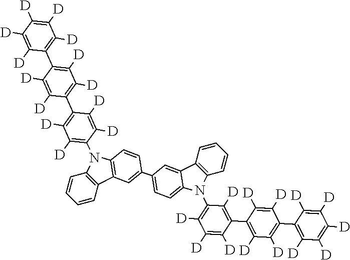 | 82 |
| 2-60 | 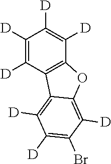 | 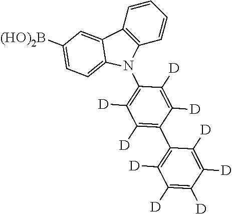 | 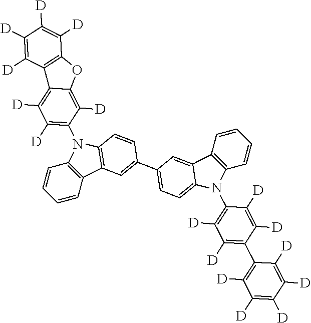 | 82 |

### <Preparation Example 6> Preparation of Compound 2-61 (F)

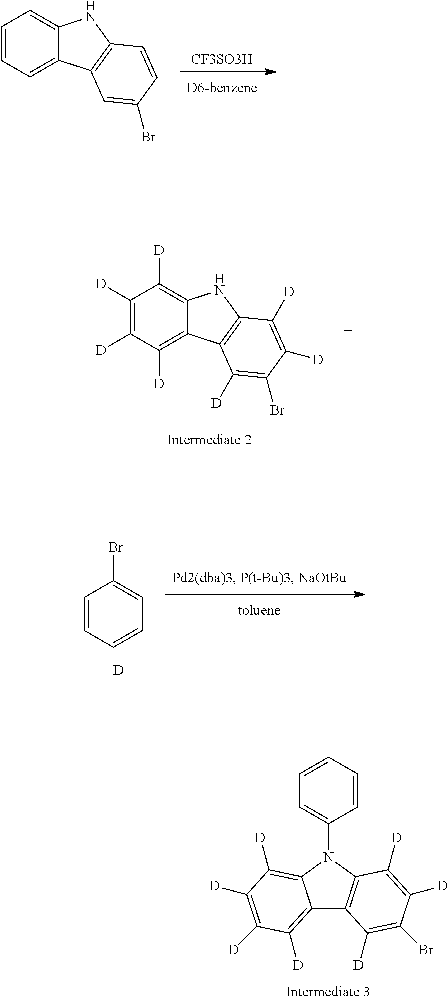

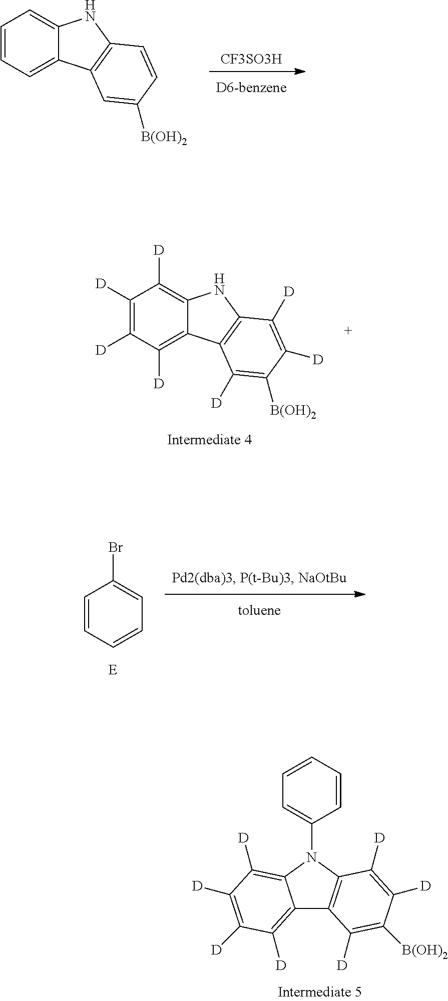 [SMILES: OB(O)c1ccc2[nH]c3ccccc3c2c1 ; "CF3SO3H" ; [2H]c1c([2H])c([2H])c2c([nH]c3c([2H])c([2H])c(B(O)O)c([2H])c32)c1[2H] ; Brc1ccccc1 ; [2H]c1c([2H])c([2H])c2c(c1[2H])c1c([2H])c(B(O)O)c([2H])c([2H])c1n2-c1ccccc1]

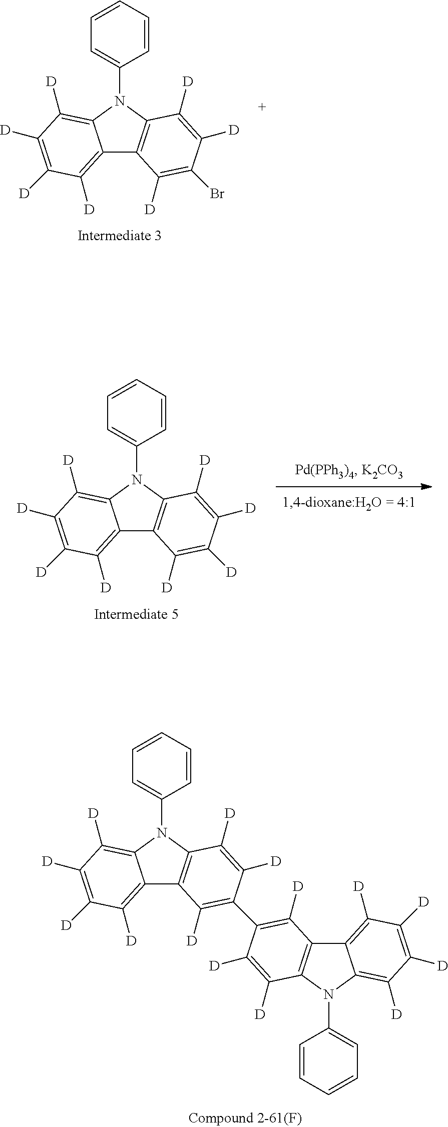 [SMILES: [2H]c1c([2H])c([2H])c2c(c1[2H])c1c([2H])c(Br)c([2H])c([2H])c1n2-c1ccccc1 ; [2H]c1c([2H])c([2H])c2c(c1[2H])c1c([2H])c([2H])c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

### Synthesis Method of Intermediate 2

After 3-bromo-9H-carbazole (10 g, 40.23 mmol) and 1000 ml of D6-benzene were put into a reaction flask, CF₃SO₃H (170 g, 1075 mmol) was added thereto, and then the resulting mixture was stirred at 50° C. When the reaction was completed, the resulting product was neutralized with D₂O, then extracted with MC and an aqueous Na₂CO₃ solution, and then column-purified to obtain Intermediate 2. (10 g, 98%)

### Synthesis Method of Intermediate 3

After Intermediate 2 (10 g, 39.5 mmol), bromobenzene (D) (12.4 g, 79 mmol), Pd₂(dba)₃ (1.81 g, 1.98 mmol), P (t-Bu)₃ (1.93 mL, 7.9 mmol), and NaOtBu (11.4 g, 118.51 mmol) were put into a reaction flask, toluene (100 ml) was added thereto, and then the resulting mixture was heated at 135° C. for 10 hours. After the reaction was completed, extraction was performed with MC and H₂O, and then column purification was performed to obtain Intermediate 3. (11 g, 84%)

### Synthesis Method of Intermediate 4

After 9H-carbazol-3-ylboronic acid (10 g, 47.3 mmol) and 1000 ml of D6-benzene were put into a reaction flask, CF₃SO₃H (170 g, 1075 mmol) was added thereto, and then the resulting mixture was stirred at 50° C. When the reaction was completed, the resulting product was neutralized with D₂O, then extracted with MC and an aqueous Na₂CO₃ solution, and then column-purified to obtain Intermediate 4. (9 g, 87%)

### Synthesis Method of Intermediate 5

After Intermediate 4 (9 g, 41.3 mmol), bromobenzene (E) (12.9 g, 82.5 mmol), Pd₂(dba)₃ (1.89 g, 2.06 mmol), P(t-Bu)₃ (2 mL, 8.25 mmol), and NaOtBu (7.93 g, 82.54 mmol) were put into a reaction flask, toluene (100 ml) was added thereto, and then the resulting mixture was heated at 135° C. for 10 hours. After the reaction was completed, extraction was performed with MC and H₂O, and then column purification was performed to obtain Intermediate 5. (10 g, 82%)

### Synthesis Method of Compound 2-61(F)

After Intermediate 3 (10 g, 30.37 mmol), Intermediate (17.87 g, 60.75 mmol), Pd(PPh₃)₄(1.39 g, 1.52 mmol), and K₂CO₃ (12.59 g, 91.13 mmol) were put into a reaction flask, a mixture of 1,4-dioxane/water (140 ml/35 ml) was heated at 120° C. for 4 hours. After the reaction was completed, the temperature was lowered to room temperature, and then the resulting solid was washed with distilled water and methanol (MeOH) to obtain Compound 2-61(F). (13 g, 85%)

The following Compound (F) was synthesized in the same manner as in Preparation Example 6 using compounds in the following Table 6 instead of D and E in Preparation Example 6.

TABLE 6

| | | | | Yield |
|---|---|---|---|---|
| Compound | D | E | F | (%) |
| 2-62 | 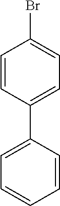 | 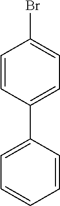 | 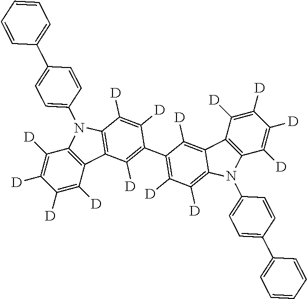 | 82 |
| 2-63 | 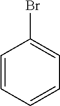 | 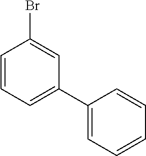 | 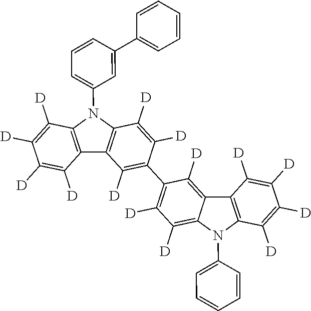 | 84 |
| 2-64 | 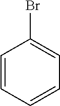 | 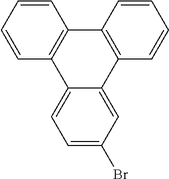 | 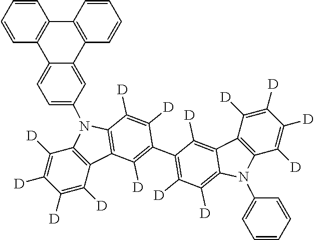 | 80 |
| 2-65 | 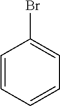 | 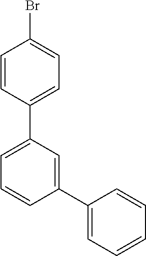 | 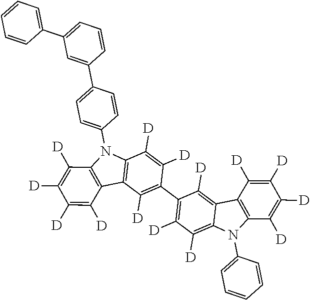 | 81 |
| 2-66 | 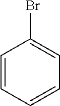 | 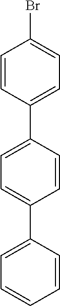 | 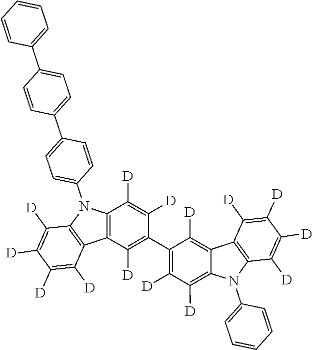 | 82 |
| 2-68 | 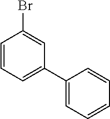 | 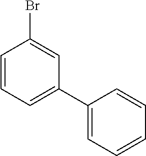 | 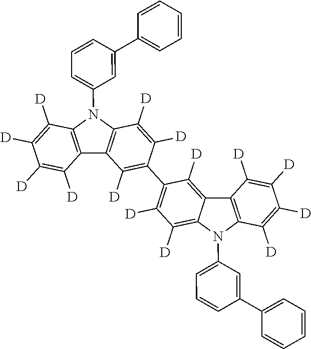 | 84 |
| 2-69 | 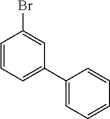 | 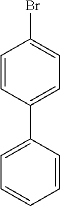 | 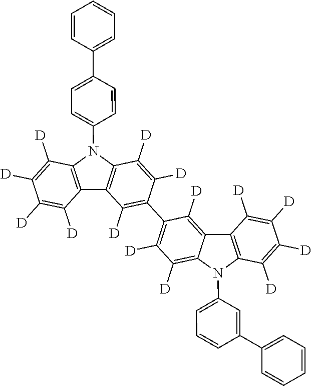 | 80 |
| 2-70 | 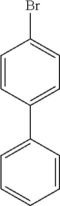 | 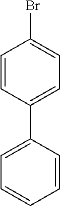 | 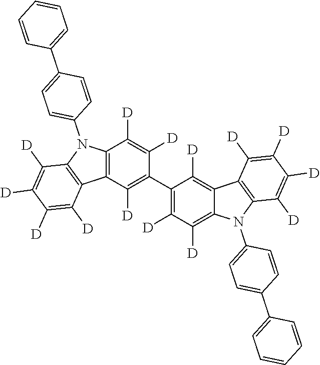 | 81 |
| 2-74 | 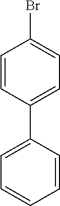 | 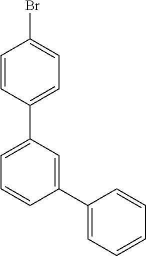 | 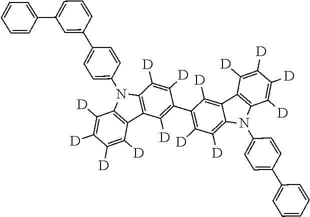 | 81 |
| 2-75 | 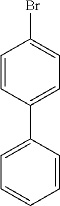 | 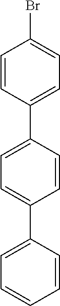 | 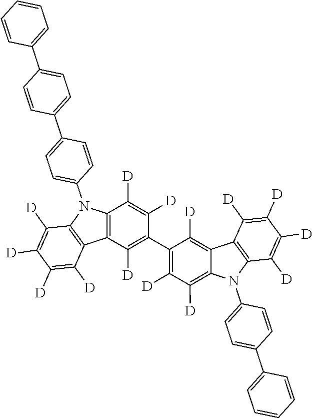 | 80 |
| 2-81 | 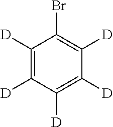 | 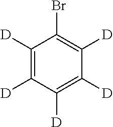 | 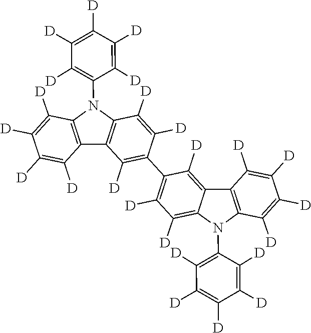 | 83 |

### <Preparation Example 7> Preparation of Compound 2-81 (H)

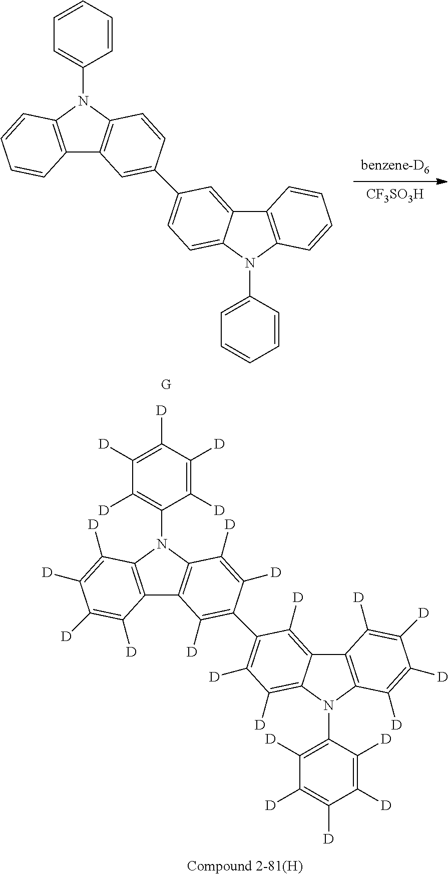

Compound G (10 g, 1 eq.), D₆-benzene (500 g, 287.93 eq.), and CF₃SO₃H (245 g, 75.04 eq.) were put into a reaction flask, and then reacted at 50° C. for 1 hour. When the reaction was completed, H₂O was added thereto for neutralization, then the resulting product was extracted with MC and H₂O, and then the organic material layer was column-purified to obtain Compound 2-81(H). (7 g, 66%) The following Compound (H) was synthesized in the same manner as in Preparation Example 7 using compounds in the following Table 7 instead of Compound G in Preparation Example 7.

TABLE 7

| Compound | | | Yield |
|---|---|---|---|
| No. | G | H | (%) |
| 2-81 | 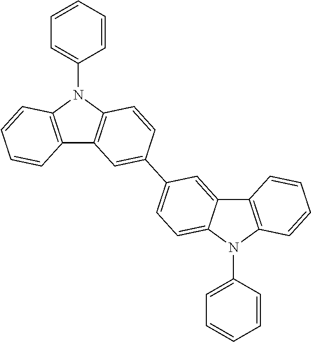 | 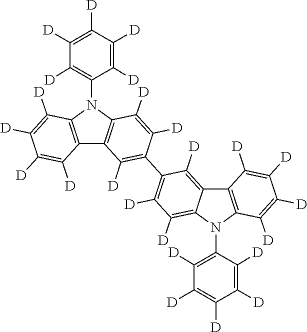 | 63 |
| 2-82 | 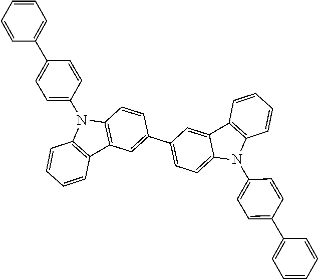 | 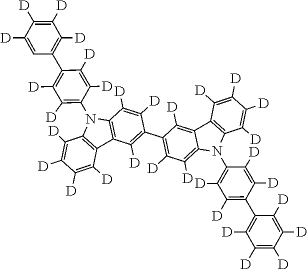 | 62 |
| 2-83 | 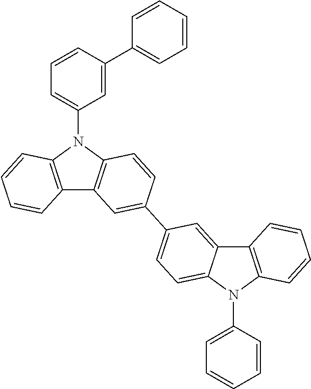 | 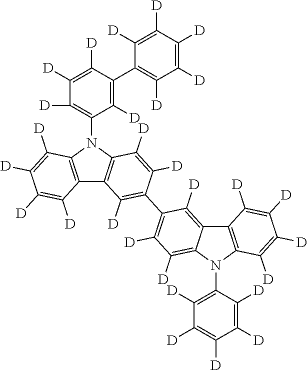 | 64 |
| 2-85 | 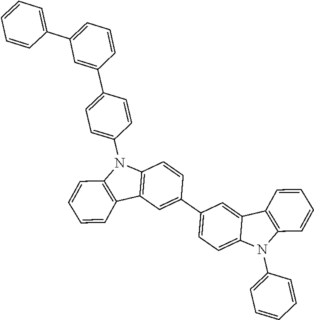 | 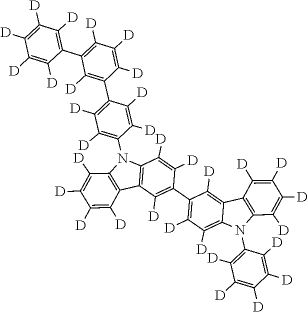 | 60 |
| 2-86 | 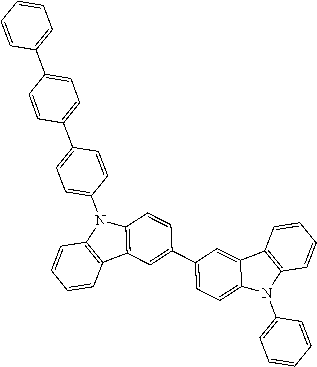 | 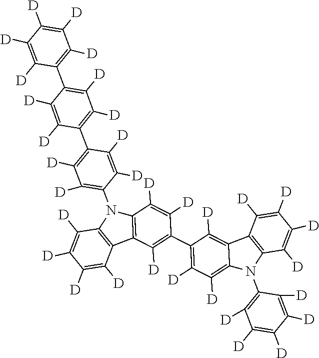 | 61 |
| 2-87 | 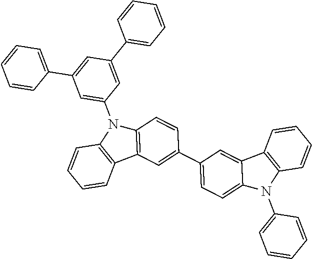 | 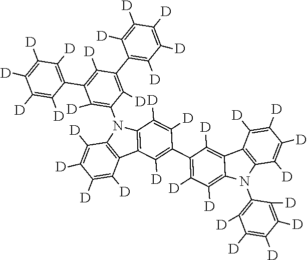 | 61 |
| 2-88 | 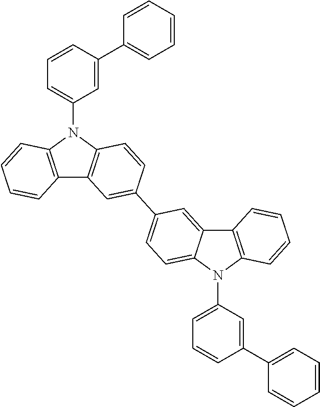 | 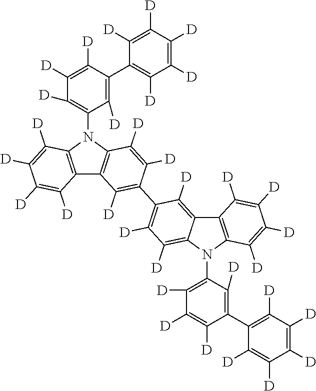 | 62 |
| 2-89 | 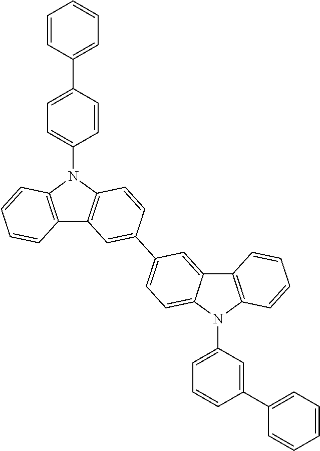 | 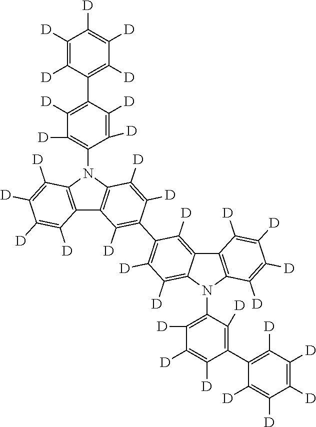 | 62 |
| 2-90 | 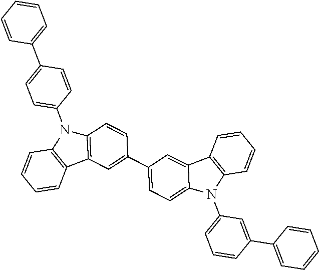 | 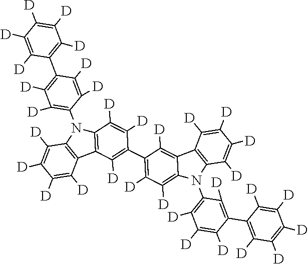 | 64 |
| 2-92 | 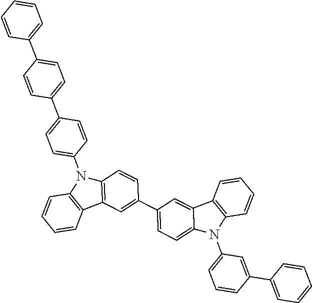 | 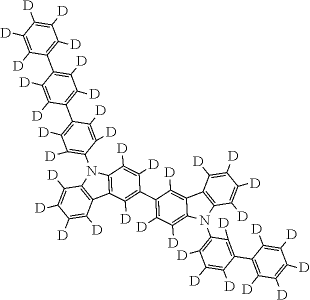 | 60 |
| 2-102 | 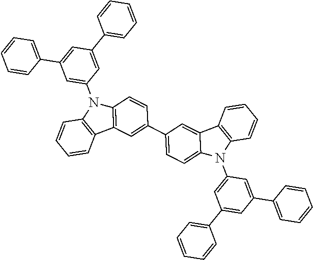 | 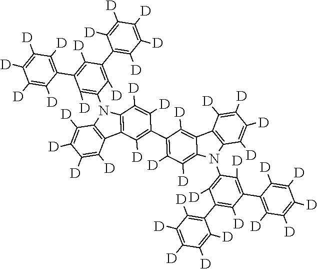 | 61 |

Compounds were prepared by the methods in the Preparation Examples, and the synthesis confirmation results thereof are shown in Tables 8 and 9. Table 8 shows the measured values of 1H NMR (CDCl₃, 300 MHz), and Table 9 shows the measured values of field desorption mass spectrometry (FD-MS).

TABLE 8

| Compound | ¹H NMR (CDCl₃, 300 MHz) |
|---|---|
| 1-1 | δ = 8.28 (m, 2H), 7.85-7.75 (m, 8H), 7.66-7.62 (m, 3H), 7.51- |
| | 7.32 (m, 10H) |
| 1-6 | δ = 8.24 (m, 1H), 7.89-7.76 (m, 14H), 7.63-7.41 (m, 10H), 7.40- |
| | 7.3 (m, 2H) |
| 1-11 | δ = 7.95-7.66 (m, 13H), 7.52-7.32 (m, 18H) |
| 1-17 | δ = 7.95-7.81 (m, 9H), 7.66-7.64 (m, 2H), 7.52-7.25 (m, 16H) |
| 1-29 | δ = 8.45-8.41 (m, 2H), 7.98-7.95 (m, 2H), 7.85-7.71 (m, 7H), |
| | 7.58-7.41 (m, 14H), 7.25 (m, 2H) |
| 1-32 | δ = 8.45-8.41 (m, 2H), 7.98-7.72 (m, 10H), 7.58-7.38 (m, 12H), |
| | 7.28 (m, 1H), 1.72 (s, 6H) |
| 1-33 | δ = 8.45-8.41 (m, 2H), 7.98-7.95 (m, 3H), 7.85-7.75 (m, 5H), |
| | 7.64-7.51 (m, 15H), 7.25 (m, 2H) |
| 1-37 | δ = 8.45-8.41 (m, 2H), 7.98-7.95 (m, 2H), 7.85-7.75 (m, 6H), |
| | 7.64-7.51 (m, 15H), 7.25 (m, 2H) |
| 1-41 | δ = 8.45-8.41 (m, 2H), 8.28 (m, 2H), 8.11-8.08 (m, 2H), 7.98- |
| | 7.94 (m, 2H), 7.82-7.79 (m, 5H), 7.58-7.41 (m, 10H) |
| 1-42 | δ = 8.45-8.41 (m, 2H), 8.28 (m, 2H), 8.11-8.08 (m, 2H), 8.00- |
| | 7.98 (m, 3H), 7.86-7.80 (m, 3H), 7.58-7.41 (m, 11H) |
| 1-54 | δ = 8.45-8.41 (m, 2H), 8.24 (m, 1H), 8.11-7.98 (m, 6H), 7.82- |
| | 7.80 (m, 2H), 7.70 (s, 1H), 7.58-7. 41 (m, 15H) |
| 1-60 | δ = 8.45-8.41 (m, 3H), 8.20 (m, 1H), 8.11-8.08 (m, 2H), 7.98- |
| | 7.80 (m, 6H0, 7.63-7.38 (m, 12H), 7.28 (m, 1H), 1.72 (s, 6H) |
| 1-61 | δ = 8.28 (m, 2H), 8.11-8.08 (m, 2H), 7.95-7.81 (m, 8H), 7.66 (m, |
| | 1H), 7.56-7.32 (m, 10H) |
| 1-69 | δ = 8.11-8.08 (m, 2H), 8.00 (m, 2H0, 7.89-7.81 (m, 7H), 7. 66 (m, |
| | 1H), 7.52-7.25 (m, 15H) |
| 1-70 | δ = 8.24 (m, 2H), 8.11-8.08 (m, 2H), 8.00 (m, 2H), 7.89-7.82 (m, |
| | 5H), 7.70-7.66 (m, 2H), 7.57-7.32 (m, 15H) |
| 1-81 | δ = 7.95-7.75 (m, 10H), 7.66-7.62 (m, 3H), 7.52-7.25 (m, 18H) |
| 1-93 | δ = 8.45-8.41 (m, 2H), 8.11-8.08 (m, 2H), 7.98-7.94 (m, 2H), |
| | 7.85-7.80 (m, 7H), 7.58-7.41 (m, 12H), 7.25 (m, 6H) |
| 1-99 | δ = 8.11-8.05 (m, 4H), 7.89-7.81 (m, 6H), 7.70-7.66 (m, 2H), |
| | 7.57-7.25 (m, 18H) |
| 1-109 | δ = 7.89-7.81 (m, 7H), 7.72-7.66 (m, 5H), 7.52-7.25 (m, 15H) |
| 1-112 | δ = 7.93-7.63 (m, 14H), 7.55-7.51 (m, 5H), 7.41-7.28 (m, 6H), |
| | 1.72 (s, 6H) |
| 1-123 | δ = 8.45-8.41 (m, 2H), 8.28 (m, 2H), 7.98-7.95 (m, 2H), 7.80- |
| | 7.71 (m, 5H), 7.64-7.41 (m, 12H) |
| 1-127 | δ = 8.45-8.41 (m, 2H), 7.98 (m, 1H), 7.80-7.41 (m, 28H) |
| 1-149 | δ = 7.89-7.62 (m, 11H), 7.52-7.25 (m, 16H) |
| 1-156 | δ = 8.41 (m, 1H), 8.28 (m, 2H), 8.20 (m, 1H), 7.94-7.81 (m, 5H), |
| | 7.66-7.32 (m, 14H) |
| 1-163 | δ = 8.45-8.41 (m, 2H), 8.28 (m, 2H), 7.98-7.95 (m, 2H), 7.80- |
| | 7.75 (m, 3H), 7.64-7.41 (m, 14H) |
| 1-172 | δ = 8.45-8.41 (m, 2H), 7.87-7.71 (m, 9H), 7.62-7.25 (m, 12H), |
| | 1.72 (m, 6H) |
| 2-3 | δ = 8.55 (d, 1H), 8.18-8.09 (m, 3H), 8.00-7.87 (m, 3H), 7.77 (s, |
| | 2H), 7.58-7.25 (m, 18H) |
| 2-4 | δ = 8.55 (d, 1H), 8.18-8.12 (m, 2H), 8.00-7.84 (m, 3H), 7.79- |
| | 7.77 (m, 4H), 7.68-7.25 (m, 22H) |
| 2-7 | δ = 8.55 (d, 1H), 8.18-8.09 (m, 4H), 8.00-7.94 (m, 2H), 7.87 (m, |
| | 1H), 7.77 (m, 2H), 7.69-7.63 (m, 2H), 7.52-7.25 (m, 20H) |
| 2-27 | δ = 8.55 (m, 1H), 8.18-8.09 (m, 4h), 8.00-7.94 (m, 2H), 7.77 (m, |
| | 2H) |
| 2-28 | δ = 8.55 (m, 1H), 8.18-8.09 (m, 3H), 8.00-8.79 (m, 2H), 7.87 (m, |
| | 1H), 7.79-7.77 (m, 4H), 7.69-7.63 (m, 4H), 7.52-7.25 (m, 12H) |
| 2-29 | δ = 8.55 (m, 1H), 8.18-8.09 (m, 3H), 8.00-7.94 (m, 2H0, 7.87 (m, |
| | 1H), 7.87 (m, 1H), 7.79-7.77 (m, 4H), 7.69-7.63 (m, 4H), 7.52- |
| | 7.25 (m, 21H) |
| 2-32 | δ = 8.55 (m, 1H), 8.18-8.12 (m, 2H), 8.00-7.87 (m, 3H), 7.79- |
| | 7.77 (m, 6H), 7.69-7.63 (m, 6H), 7.52-7.25 (m, 14H) |
| 2-34 | δ = 8.55 (m, 1H), 8.18-8.12 (m, 2H), 8.00-7.87 (m, 3H), 7.79- |
| | 7.77 (m, 6H), 7.67-7.63 (m, 6H), 7.52-7.25 (m, 18H) |
| 2-38 | δ = 8.55 (m, 1H), 8.18-8.12 (m, 2H), 8.05-7.87 (m, 6H), 7.79- |
| | 7.77 (m, 4H), 7.69-7.63 (m, 4H), 7.52-7.25 (m, 23H) |
| 2-46 | δ = 8.55 (d, 1H), 8.18-8.12 (m, 2H), 8.00-7.87 (m, 3H), 7.77- |
| | 7.63 (m, 4H), 7.50 (m, 1H), 7.33-7.25 (m, 3H) |
| 2-49 | δ = 8.55 (d, 1H), 8.18-8.12 (m, 2H), 8.00-7.87 (m, 3H), 7.77- |
| | 7.63 (m, 4H), 7.50 (m, 1H), 7.33-7.25 (m, 3H) |
| 2-62 | δ = 7.79 (m, 4H), 7.68 (m, 4H), 7.52-7.41 (m, 10H) |
| 2-65 | δ = 7.79 (m, 2H), 7.70-7.68 (m, 3H), 7.58-7.41 (m, 13H) |
| 2-70 | δ = 7.79 (m, 4H), 7.68 (m, 4H), 7.52-7.41 (m, 10H) |

TABLE 9

| Compound | FD-Mass | Compound | FD-Mass |
|---|---|---|---|
| 1-1 | m/z = 565.62 | 1-2 | m/z = 565.62 |
| | (C39H23N3O2 = 565.28) | | (C39H23N3O2 = 565.28) |
| 1-3 | m/z = 565.62 | 1-4 | m/z = 565.62 |
| | (C39H23N3O2 = 565.28) | | (C39H23N3O2 = 565.28) |
| 1-5 | m/z = 641.71 | 1-6 | m/z = 641.71 |
| | (C51H31N3O2 = 641.21) | | (C51H31N3O2 = 641.21) |
| 1-7 | m/z = 717.81 | 1-8 | m/z = 681.78 |
| | (C51H31N3O2 = 717.24) | | (C48H31N3O2 = 681.24) |
| 1-9 | m/z = 641.71 | 1-10 | m/z = 641.71 |
| | (C51H31N3O2 = 641.21) | | (C51H31N3O2 = 641.21) |
| 1-11 | m/z = 717.81 | 1-12 | m/z = 681.78 |
| | (C51H31N3O2 = 717.24) | | (C48H31N3O2 = 681.24) |
| 1-13 | m/z = 641.71 | 1-14 | m/z = 641.71 |
| | (C51H31N3O2 = 641.21) | | (C51H31N3O2 = 641.21) |
| 1-15 | m/z = 717.81 | 1-16 | m/z = 681.78 |
| | (C51H31N3O2 = 717.24) | | (C48H31N3O2 = 681.24) |
| 1-17 | m/z = 641.71 | 1-18 | m/z = 641.71 |
| | (C51H31N3O2 = 641.21) | | (C51H31N3O2 = 641.21) |
| 1-19 | m/z = 717.81 | 1-20 | m/z = 681.78 |
| | (C51H31N3O2 = 717.24) | | (C48H31N302 = 681.24) |
| 1-21 | m/z = 581.68 | 1-22 | m/z = 581.68 |
| | (C39H23N3OS = 581.16) | | (C39H23N3OS = 581.16) |
| 1-23 | m/z = 581.68 | 1-24 | m/z = 581.68 |
| | (C39H23N3OS = 581.16) | | (C39H23N3OS = 581.16) |
| 1-25 | m/z = 657.78 | 1-26 | m/z = 657.78 |
| | (C45H17N3OS = 657.19) | | (C45H17N3OS = 657.19) |
| 1-27 | m/z = 733.88 | 1-28 | m/z = 697.84 |
| | (C51H31N3OS = 733.22) | | (C48H31N3OS = 697.22) |
| 1-29 | m/z = 657.78 | 1-30 | m/z = 657.78 |
| | (C45H17N3OS = 657.19) | | (C45H17N3OS = 657.19) |
| 1-31 | m/z = 733.88 | 1-32 | m/z = 697.84 |
| | (C51H31N3OS = 733.22) | | (C48H31N3OS = 697.22) |
| 1-33 | m/z = 657.78 | 1-34 | m/z = 657.78 |
| | (C45H17N3OS = 657.19) | | (C45H17N3OS = 657.19) |
| 1-35 | m/z = 733.88 | 1-36 | m/z = 697.84 |
| | (C51H31N3OS = 733.22) | | (C48H31N3OS = 697.22) |
| 1-37 | m/z = 657.78 | 1-38 | m/z = 657.78 |
| | (C45H17N3OS = 657.19) | | (C45H17N3OS = 657.19) |
| 1-39 | m/z = 733.88 | 1-40 | m/z = 697.84 |
| | (C51H31N3OS = 733.22) | | (C48H31N3OS = 697.22) |
| 1-41 | m/z = 597.75 | 1-42 | m/z = 597.75 |
| | (C39H23N3S2 = 597.13) | | (C39H23N3S2 = 597.13) |
| 1-43 | m/z = 597.75 | 1-44 | m/z = 597.75 |
| | (C39H23N3S2 = 597.13) | | (C39H23N3S2 = 597.13) |
| 1-45 | m/z = 673.85 | 1-46 | m/z = 673.85 |
| | (C45H27N3S2 = 673.16) | | (C45H27N3S2 = 673.16) |
| 1-47 | m/z = 749.94 | 1-48 | m/z = 713.91 |
| | (C51H31N3S2 = 749.20) | | (C48H31N3S2 = 713.20) |
| 1-49 | m/z = 673.85 | 1-50 | m/z = 673.85 |
| | (C45H27N3S2 = 673.16) | | (C45H27N3S2 = 673.16) |
| 1-51 | m/z = 749.94 | 1-52 | m/z = 713.91 |
| | (C51H31N3S2 = 749.20) | | (C48H31N3S2 = 713.20) |
| 1-53 | m/z = 673.85 | 1-54 | m/z = 673.85 |
| | (C45H27N3S2 = 673.16) | | (C45H27N3S2 = 673.16) |
| 1-55 | m/z = 749.94 | 1-56 | m/z = 713.91 |
| | (C51H31N3S2 = 749.20) | | (C48H31N3S2 = 713.20) |
| 1-57 | m/z = 673.85 | 1-58 | m/z = 673.85 |
| | (C45H27N3S2 = 673.16) | | (C45H27N3S2 = 673.16) |
| 1-59 | m/z = 749.94 | 1-60 | m/z = 713.91 |
| | (C51H31N3S2 = 749.20) | | (C48H31N3S2 = 713.20) |
| 1-61 | m/z = 581.68 | 1-62 | m/z = 581.68 |
| | (C39H23N3OS = 581.16) | | (C39H23N3OS = 581.16) |
| 1-63 | m/z = 581.68 | 1-64 | m/z = 581.68 |
| | (C39H23N3OS = 581.16) | | (C39H23N3OS = 581.16) |
| 1-65 | m/z = 657.78 | 1-66 | m/z = 657.78 |
| | (C45H17N3OS = 657.19) | | (C45H17N3OS = 657.19) |
| 1-67 | m/z = 733.88 | 1-68 | m/z = 697.84 |
| | (C51H31N3OS = 733.22) | | (C48H31N3OS = 697.22) |
| 1-69 | m/z = 657.78 | 1-70 | m/z = 657.78 |
| | (C45H17N3OS = 657.19) | | (C45H17N3OS = 657.19) |
| 1-71 | m/z = 733.88 | 1-72 | m/z = 697.84 |
| | (C51H31N3OS = 733.22) | | (C48H31N3OS = 697.22) |
| 1-73 | m/z = 657.78 | 1-74 | m/z = 657.78 |
| | (C45H17N3OS = 657.19) | | (C45H17N3OS = 657.19) |
| 1-75 | m/z = 733.88 | 1-76 | m/z = 697.84 |
| | (C51H31N3OS = 733.22) | | (C48H31N3OS = 697.22) |
| 1-77 | m/z = 657.78 | 1-78 | m/z = 657.78 |
| | (C45H17N3OS = 657.19) | | (C45H17N3OS = 657.19) |
| 1-79 | m/z = 733.88 | 1-80 | m/z = 697.84 |
| | (C51H31N3OS = 733.22) | | (C48H31N3OS = 697.22) |
| 1-81 | m/z = 717.81 | 1-82 | m/z = 717.81 |
| | (C51H31N3O2 = 717.24) | | (C51H31N3O2 = 717.24) |
| 1-83 | m/z = 717.81 | 1-84 | m/z = 717.81 |
| | (C51H31N3O2 = 717.24) | | (C51H31N3O2 = 717.24) |
| 1-85 | m/z = 717.81 | 1-86 | m/z = 717.81 |
| | (C51H31N3O2 = 717.24) | | (C51H31N3O2 = 717.24) |
| 1-87 | m/z = 717.81 | 1-88 | m/z = 717.81 |
| | (C51H31N3O2 = 717.24) | | (C51H31N3O2 = 717.24) |
| 1-89 | m/z = 733.88 | 1-90 | m/z = 733.88 |
| | (C51H31N3OS = 733.22) | | (C51H31N3OS = 733.22) |
| 1-91 | m/z = 733.88 | 1-92 | m/z = 733.88 |
| | (C51H31N3OS = 733.22) | | (C51H31N3OS = 733.22) |
| 1-93 | m/z = 749.94 | 1-94 | m/z = 749.94 |
| | (C51H31N3S2 = 749.20) | | (C51H31N3S2 = 749.20) |
| 1-95 | m/z = 749.94 | 1-96 | m/z = 749.94 |
| | (C51H31N3S2 = 749.20) | | (C51H31N3S2 = 749.20) |
| 1-97 | m/z = 733.88 | 1-98 | m/z = 733.88 |
| | (C51H31N3OS = 733.22) | | (C51H31N3OS = 733.22) |
| 1-99 | m/z = 733.88 | 1-100 | m/z = 733.88 |
| | (C51H31N3OS = 733.22) | | (C51H31N3OS = 733.22) |
| 1-101 | m/z = 565.62 | 1-102 | m/z = 565.62 |
| | (C39H23N3O2 = 565.28) | | (C39H23N3O2 = 565.28) |
| 1-103 | m/z = 565.62 | 1-104 | m/z = 565.62 |
| | (C39H23N3O2 = 565.28) | | (C39H23N3O2 = 565.28) |
| 1-105 | m/z = 641.71 | 1-106 | m/z = 641.71 |
| | (C51H31N3O2 = 641.21) | | (C51H31N3O2 = 641.21) |
| 1-107 | m/z = 717.81 | 1-108 | m/z = 717.81 |
| | (C51H31N3O2 = 717.24) | | (C51H31N3O2 = 717.24) |
| 1-109 | m/z = 641.71 | 1-110 | m/z = 641.71 |
| | (C51H31N3O2 = 641.21) | | (C51H31N3O2 = 641.21) |
| 1-111 | m/z = 717.81 | 1-112 | m/z = 717.81 |
| | (C51H31N3O2 = 717.24) | | (C51H31N3O2 = 717.24) |
| 1-113 | m/z = 581.68 | 1-114 | m/z = 581.68 |
| | (C39H23N3OS = 581.16) | | (C39H23N3OS = 581.16) |
| 1-115 | m/z = 581.68 | 1-116 | m/z = 581.68 |
| | (C39H23N3OS = 581.16) | | (C39H23N3OS = 581.16) |
| 1-117 | m/z = 657.78 | 1-118 | m/z = 657.78 |
| | (C45H17N3OS = 657.19) | | (C45H17N3OS = 657.19) |
| 1-119 | m/z = 733.88 | 1-120 | m/z = 697.84 |
| | (C51H31N3OS = 733.22) | | (C48H31N3OS = 697.22) |
| 1-121 | m/z = 581.68 | 1-122 | m/z = 581.68 |
| | (C39H23N3OS = 581.16) | | (C39H23N3OS = 581.16) |
| 1-123 | m/z = 581.68 | 1-124 | m/z = 581.68 |
| | (C39H23N3OS = 581.16) | | (C39H23N3OS = 581.16) |
| 1-125 | m/z = 657.78 | 1-126 | m/z = 657.78 |
| | (C45H17N3OS = 657.19) | | (C45H17N3OS = 657.19) |
| 1-127 | m/z = 733.88 | 1-128 | m/z = 697.84 |
| | (C51H31N3OS = 733.22) | | (C48H31N3OS = 697.22) |
| 1-129 | m/z = 657.78 | 1-130 | m/z = 657.78 |
| | (C45H17N3OS = 657.19) | | (C45H17N3OS = 657.19) |
| 1-131 | m/z = 733.88 | 1-132 | m/z = 697.84 |
| | (C51H31N3OS = 733.22) | | (C48H31N3OS = 697.22) |
| 1-133 | m/z = 597.75 | 1-134 | m/z = 597.75 |
| | (C39H23N3S2 = 597.13) | | (C39H23N3S2 = 597.13) |
| 1-135 | m/z = 597.75 | 1-136 | m/z = 597.75 |
| | (C39H23N3S2 = 597.13) | | (C39H23N3S2 = 597.13) |
| 1-137 | m/z = 673.85 | 1-138 | m/z = 673.85 |
| | (C45H27N3S2 = 673.16) | | (C45H27N3S2 = 673.16) |
| 1-139 | m/z = 749.94 | 1-140 | m/z = 713.91 |
| | (C51H31N3S2 = 749.20) | | (C48H31N3S2 = 713.20) |
| 1-141 | m/z = 565.62 | 1-142 | m/z = 565.62 |
| | (C39H23N3O2 = 565.28) | | (C39H23N3O2 = 565.28) |
| 1-143 | m/z = 565.62 | 1-144 | m/z = 565.62 |
| | (C39H23N3O2 = 565.28) | | (C39H23N3O2 = 565.28) |
| 1-145 | m/z = 641.71 | 1-146 | m/z = 641.71 |
| | (C51H31N3O2 = 641.21) | | (C51H31N3O2 = 641.21) |
| 1-147 | m/z = 717.81 | 1-148 | m/z = 717.81 |
| | (C51H31N3O2 = 717.24) | | (C51H31N3O2 = 717.24) |
| 1-149 | m/z = 641.71 | 1-150 | m/z = 641.71 |
| | (C51H31N3O2 = 641.21) | | (C51H31N3O2 = 641.21) |
| 1-151 | m/z = 717.81 | 1-152 | m/z = 717.81 |
| | (C51H31N3O2 = 717.24) | | (C51H31N3O2 = 717.24) |
| 1-153 | m/z = 581.68 | 1-154 | m/z = 581.68 |
| | (C39H23N3OS = 581.16) | | (C39H23N3OS = 581.16) |
| 1-155 | m/z = 581.68 | 1-156 | m/z = 581.68 |
| | (C39H23N3OS = 581.16) | | (C39H23N3OS = 581.16) |
| 1-157 | m/z = 657.78 | 1-158 | m/z = 657.78 |
| | (C45H17N3OS = 657.19) | | (C45H17N3OS = 657.19) |
| 1-159 | m/z = 733.88 | 1-160 | m/z = 697.84 |
| | (C51H31N3OS = 733.22) | | (C48H31N3OS = 697.22) |
| 1-161 | m/z = 581.68 | 1-162 | m/z = 581.68 |
| | (C39H23N3OS = 581.16) | | (C39H23N3OS = 581.16) |
| 1-163 | m/z = 581.68 | 1-164 | m/z = 581.68 |
| | (C39H23N3OS = 581.16) | | (C39H23N3OS = 581.16) |
| 1-165 | m/z = 657.78 | 1-166 | m/z = 657.78 |
| | (C45H17N3OS = 657.19) | | (C45H17N3OS = 657.19) |
| 1-167 | m/z = 733.88 | 1-168 | m/z = 697.84 |
| | (C51H31N3OS = 733.22) | | (C48H31N3OS = 697.22) |
| 1-169 | m/z = 657.78 | 1-170 | m/z = 657. 78 |
| | (C45H17N3OS = 657.19) | | (C45H17N3OS = 657.19) |
| 1-171 | m/z = 733.88 | 1-172 | m/z = 697.84 |
| | (C51H31N3OS = 733.22) | | (C48H31N3OS = 697.22) |
| 1-173 | m/z = 597.75 | 1-174 | m/z = 597.75 |
| | (C39H23N3S2 = 597.13) | | (C39H23N3S2 = 597.13) |
| 1-175 | m/z = 597.75 | 1-176 | m/z = 597.75 |
| | (C39H23N3S2 = 597.13) | | (C39H23N3S2 = 597.13) |
| 1-177 | m/z = 673.85 | 1-178 | m/z = 673.85 |
| | (C45H27N3S2 = 673.16) | | (C45H27N3S2 = 673.16) |
| 1-179 | m/z = 749.94 | 1-180 | m/z = 713.91 |
| | (C51H31N3S2 = 749.20) | | (C48H31N3S2 = 713.20) |
| 1-181 | m/z = 576.69 | 1-182 | m/z = 588.76 |
| | (C39H12D11N3O2 = 576.25) | | (C39D23N3O2 = 588.32) |
| 1-183 | m/z = 576.69 | 1-184 | m/z = 588.76 |
| | (C39H12D11N3O2 = 576.25) | | (C39D23N3O2 = 588.32) |
| 1-185 | m/z = 652.78 | 1-186 | m/z = 668.88 |
| | (C45H16D11N3O2 = 652.28) | | (C45D27N3O2 = 668.38) |
| 1-187 | m/z = 652.78 | 1-188 | m/z = 652.78 |
| | (C45H16D11N3O2 = 652.28) | | (C45H16D11N3O2 = 652.28) |
| 1-189 | m/z = 592.75 | 1-190 | m/z = 604.83 |
| | (C39H12D11N3OS = 592.23) | | (C39D23N3OS = 604.30) |
| 1-191 | m/z = 592.75 | 1-192 | m/z = 604.83 |
| | (C39H12D11N3OS = 592.23) | | (C39D23N3OS = 604.30) |
| 1-193 | m/z = 668.85 | 1-194 | m/z = 684.95 |
| | (C45H16D11N3OS = 668.26) | | (C45D27N3OS = 684.36) |
| 1-195 | m/z = 668.85 | 1-196 | m/z = 668.85 |
| | (C45H16D11N3OS = 668.26) | | (C45H16D11N3OS = 668.26) |
| 1-197 | m/z = 608.82 | 1-198 | m/z = 620.89 |
| | (C39H12D11N3S2 = 608.20) | | (C39D23N3S2 = 620.28) |
| 1-199 | m/z = 684.91 | 1-200 | m/z = 620.89 |
| | (C45H16D11N3S2 = 684.23) | | (C39D23N3S2 = 620.28) |
| 1-201 | m/z = 576.69 | 1-202 | m/z = 588.76 |
| | (C39H12D11N3O2 = 576.25) | | (C39D23N3O2 = 588.32) |
| 1-203 | m/z = 576.69 | 1-204 | m/z = 588.76 |
| | (C39H12D11N3O2 = 576.25) | | (C39D23N3O2 = 588.32) |
| 1-205 | m/z = 652.78 | 1-206 | m/z = 668.88 |
| | (C45H16D11N3O2 = 652.28) | | (C45D27N3O2 = 668.38) |
| 1-207 | m/z = 652.78 | 1-208 | m/z = 652.78 |
| | (C45H16D11N3O2 = 652.28) | | (C45H16D11N3O2 = 652.28) |
| 1-209 | m/z = 592.75 | 1-210 | m/z = 604.83 |
| | (C39H12D11N3OS = 592.23) | | (C39D23N3OS = 604.30) |
| 1-211 | m/z = 592.75 | 1-212 | m/z = 604.83 |
| | (C39H12D11N3OS = 592.23) | | (C39D23N3OS = 604.30) |
| 1-213 | m/z = 576.69 | 1-214 | m/z = 588.76 |
| | (C39H12D11N3O2 = 576.25) | | (C39D23N3O2 = 588.32) |
| 1-215 | m/z = 576.69 | 1-216 | m/z = 588.76 |
| | (C39H12D11N3O2 = 576.25) | | (C39D23N3O2 = 588.32) |
| 1-217 | m/z = 652.78 | 1-218 | m/z = 668.88 |
| | (C45H16D11N3O2 = 652.28) | | (C45D27N3O2 = 668.38) |
| 1-219 | m/z = 652.78 | 1-220 | m/z = 652.78 |
| | (C45H16D11N3O2 = 652.28) | | (C45H16D11N3O2 = 652.28) |
| 2-1 | m/z = 484.59 | 2-2 | m/z = 560.69 |
| | (C36H24N2 = 484.19) | | (C42H28N2 = 560.23) |
| 2-3 | m/z = 560.69 | 2-4 | m/z = 560.69 |
| | (C42H28N2 = 560.23) | | (C42H28N2 = 560.23) |
| 2-5 | m/z = 636.78 | 2-6 | m/z = 636.78 |
| | (C48H32N2 = 636.26) | | (C48H32N2 = 636.26) |
| 2-7 | m/z = 636.78 | 2-8 | m/z = 543.65 |
| | (C48H32N2 = 636.26) | | (C40H26N2 = 543.21) |
| 2-9 | m/z = 543.65 | 2-10 | m/z = 600.75 |
| | (C40H26N2 = 543.21) | | (C45H35N2 = 600.26) |
| 2-11 | m/z = 600.75 | 2-12 | m/z = 724.89 |
| | (C45H35N2 = 600.26) | | (C55H36N2 = 724.29) |
| 2-13 | m/z = 724.89 | 2-14 | m/z = 724.89 |
| | (C55H36N2 = 724.29) | | (C55H36N2 = 724.29) |
| 2-15 | m/z = 724.89 | 2-16 | m/z = 634.77 |
| | (C55H36N2 = 724.29) | | (C48H30N2 = 634.24) |
| 2-17 | m/z = 509.60 | 2-18 | m/z = 742.98 |
| | (C37H23N3 = 509.19) | | (C54H38N2Si = 742.28) |
| 2-19 | m/z = 636.78 | 2-20 | m/z = 636.78 |
| | (C48H32N2 = 636.26) | | (C48H32N2 = 636.26) |
| 2-21 | m/z = 636.78 | 2-22 | m/z = 712.88 |
| | (C48H32N2 = 636.26) | | (C54H36N2 = 712.29) |
| 2-23 | m/z = 712.88 | 2-24 | m/z = 712.88 |
| | (C54H36N2 = 712.29) | | (C54H36N2 = 712.29) |
| 2-25 | m/z = 710.86 | 2-26 | m/z = 712.88 |
| | (C54H34N2 = 710.27) | | (C54H36N2 = 712.29) |
| 2-27 | m/z = 712.88 | 2-28 | m/z = 712.88 |
| | (C54H36N2 = 712.29) | | (C54H36N2 = 712.29) |
| 2-29 | m/z = 712.88 | 2-30 | m/z = 712.88 |
| | (C54H36N2 = 712.29) | | (C54H36N2 = 712.29) |
| 2-31 | m/z = 710.86 | 2-32 | m/z = 636.78 |
| | (C54H34N2 = 710.27) | | (C48H32N2 = 636.26) |
| 2-33 | m/z = 712.88 | 2-34 | m/z = 712.88 |
| | (C54H36N2 = 712.29) | | (C54H36N2 = 712.29) |
| 2-35 | m/z = 788.97 | 2-36 | m/z = 686.84 |
| | (C60H40N2 = 788.32) | | (C52H34N2 = 686.27) |
| 2-37 | m/z = 788.97 | 2-38 | m/z = 788.97 |
| | (C60H40N2 = 788.32) | | (C60H40N2 = 788.32) |
| 2-39 | m/z = 686.84 | 2-40 | m/z = 686.84 |
| | (C52H34N2 = 686.27) | | (C52H34N2 = 686.27) |
| 2-41 | m/z = 494.65 | 2-42 | m/z = 654.89 |
| | (C36H14D10N2 = 494.26) | | (C48H14D18N2 = 654.37) |
| 2-43 | m/z = 574.77 | 2-44 | m/z = 650.86 |
| | (C41H14D14N2 = 574.31) | | (C48H14D16N2 = 650.34) |
| 2-45 | m/z = 654.89 | 2-46 | m/z = 654.89 |
| | (C48H14D18N2 = 654.37) | | (C48H14D18N2 = 654.37) |
| 2-47 | m/z = 654.89 | 2-48 | m/z = 654.89 |
| | (C48H14D18N2 = 654.37) | | (C48H14D18N2 = 654.37) |
| 2-49 | m/z = 654.89 | 2-50 | m/z = 654.89 |
| | (C48H14D18N2 = 654.37) | | (C48H14D18N2 = 654.37) |
| 2-51 | m/z = 735.01 | 2-52 | m/z = 735.01 |
| | (C54H14D22N2 = 734.43) | | (C54H14D22N2 = 734.43) |
| 2-53 | m/z = 730.98 | 2-54 | m/z = 735.01 |
| | (C54H14D20N2 = 730.40) | | (C54H14D22N2 = 734.43) |
| 2-55 | m/z = 735.01 | 2-56 | m/z = 815.13 |
| | (C54H14D22N2 = 734.43) | | (C60H14D26N2 = 814.48) |
| 2-57 | m/z = 815.13 | 2-58 | m/z = 815.13 |
| | (C60H14D26N2 = 814.48) | | (C60H14D26N2 = 814.48) |
| 2-59 | m/z = 815.13 | 2-60 | m/z = 666.86 |
| | (C60H14D26N2 = 814.48) | | (C48H14D16N20 = 666.34) |
| 2-61 | m/z = 498.68 | 2-62 | m/z = 650.87 |
| | (C36H10D14N2 = 498.28) | | (C48H18D14N2 = 650.34) |
| 2-63 | m/z = 574.77 | 2-64 | m/z = 648.85 |
| | (C41H14D14N2 = 574.31) | | (C48H16D14N2 = 648.33) |
| 2-65 | m/z = 650.87 | 2-66 | m/z = 650.87 |
| | (C48H18D14N2 = 650.34) | | (C48H18D14N2 = 650.34) |
| 2-67 | m/z = 650.87 | 2-68 | m/z = 650.87 |
| | (C48H18D14N2 = 650.34) | | (C48H18D14N2 = 650.34) |
| 2-69 | m/z = 650.87 | 2-70 | m/z = 650.87 |
| | (C48H18D14N2 = 650.34) | | (C48H18D14N2 = 650.34) |
| 2-71 | m/z = 726.96 | 2-72 | m/z = 726.96 |
| | (C54H22D14N2 = 726.38) | | (C54H22D14N2 = 726.38) |
| 2-73 | m/z = 724.95 | 2-74 | m/z = 726.96 |
| | (C54H20D14N2 = 724.36) | | (C54H22D14N2 = 726.38) |
| 2-75 | m/z = 726.96 | 2-76 | m/z = 803.06 |
| | (C54H22D14N2 = 726.38) | | (C60H26D14N2 = 802.41) |
| 2-77 | m/z = 803.06 | 2-78 | m/z = 803.06 |
| | (C60H26D14N2 = 802.41) | | (C60H26D14N2 = 802.41) |
| 2-79 | m/z = 803.06 | 2-80 | m/z = 803.06 |
| | (C60H26D14N2 = 802.41) | | (C60H26D14N2 = 802.41) |
| 2-81 | m/z = 508.74 | 2-82 | m/z = 668.98 |
| | (C36D24N2 = 508.34) | | (C48D32N2 = 668.46) |
| 2-83 | m/z = 588.86 | 2-84 | m/z = 664.95 |
| | (C42D28N2 = 588.40) | | (C48D30N2 = 664.43) |
| 2-85 | m/z = 668.98 | 2-86 | m/z = 668.98 |
| | (C48D32N2 = 668.46) | | (C48D32N2 = 668.46) |
| 2-87 | m/z = 668.98 | 2-88 | m/z = 668.98 |
| | (C48D32N2 = 668.46) | | (C48D32N2 = 668.46) |
| 2-89 | m/z = 668.98 | 2-90 | m/z = 668.98 |
| | (C48D32N2 = 668.46) | | (C48D32N2 = 668.46) |
| 2-91 | m/z = 749.10 | 2-92 | m/z = 749.10 |
| | (C54D36N2 = 748.51) | | (C54D36N2 = 748.51) |
| 2-93 | m/z = 745.07 | 2-94 | m/z = 749.10 |
| | (C54D34N2 = 744.49) | | (C54D36N2 = 748.51) |
| 2-95 | m/z = 749.10 | 2-96 | m/z = 829.22 |
| | (C54D36N2 = 748.51) | | (C60D40N2 = 828.57) |
| 2-97 | m/z = 829.22 | 2-98 | m/z = 829.22 |
| | (C60D40N2 = 828.57) | | (C60D40N2 = 828.57) |
| 2-99 | m/z = 829.22 | 2-100 | m/z = 680.95 |
| | (C60D40N2 = 828.57) | | (C48D30N2O = 680.42) |
| 2-101 | m/z = 697.02 | 2-102 | m/z = 829.22 |
| | (C48D30N2S = 696.40) | | (C60D40N2 = 828.57) |
| 2-103 | m/z = 632.95 | 2-104 | m/z = 713.07 |
| | (C45D32N2 = 632.46) | | (C51D36N2 = 712.51) |

### <Experimental Example 1> Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

A hole injection layer 4,4′,4″-tris[2-naphthyl(phenyl)amino]triphenylamine (2-TNATA) and a hole transport layer N,N′-di(1-naphthyl)-N,N′-diphenyl-(1,1′-biphenyl)-4,4′-diamine (NPB), which are common layers, were formed on the ITO transparent electrode (positive electrode).

A light emitting layer was thermally vacuum deposited thereon as follows. The light emitting layer was deposited to have a thickness of 360 Å by using a compound described in the following Table 10 as a host and tris (2-phenylpyridine)iridium (Ir(ppy)₃) as a green phosphorescent dopant to dope the host with Ir(ppy)₃ in an amount of 7%.

Thereafter, BCP as a hole blocking layer was deposited to have a thickness of 60 Å, and Alq₃ as an electron transport layer was deposited to have a thickness of 200 Å thereon. Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1200 Å on the electron injection layer to form a negative electrode.

Meanwhile, all the organic compounds required for manufacturing an OLED device were subjected to vacuum sublimed purification under 10⁻⁶ to 10⁻⁸ torr for each material, and used for the manufacture of OLED.

The organic light emitting devices of Comparative Examples 1 to 6 were manufactured in the same manner as in Experimental Example 1, except that the following comparative compounds were used as the host of the light emitting layer.

For the organic light emitting device manufactured as described above, electroluminescence (EL) characteristics were measured by M7000 manufactured by McScience Inc., and based on the measurement result thereof, T90 was measured by a service life measurement device (M6000) manufactured by McScience Inc., when the reference luminance was 6,000 cd/m². The results of measuring the driving voltage, light emitting efficiency, color coordinate (CIE) and service life of the organic light emitting device manufactured according to the present invention are shown in the following Table 10.

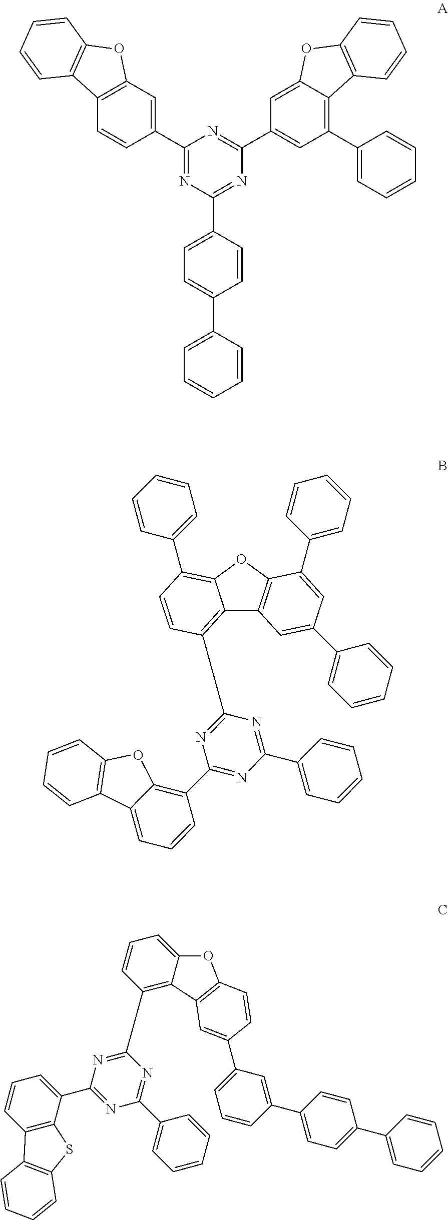 [SMILES: c1ccc(-c2ccc(-c3nc(-c4ccc5c(c4)oc4ccccc45)nc(-c4cc(-c5ccccc5)c5c(c4)oc4ccccc45)n3)cc2)cc1 ; c1ccc(-c2cc(-c3ccccc3)c3oc4c(-c5ccccc5)ccc(-c5nc(-c6ccccc6)nc(-c6cccc7c6oc6ccccc67)n5)c4c3c2)cc1 ; c1ccc(-c2ccc(-c3cccc(-c4ccc5oc6cccc(-c7nc(-c8ccccc8)nc(-c8cccc9c8sc8ccccc89)n7)c6c5c4)c3)cc2)cc1]

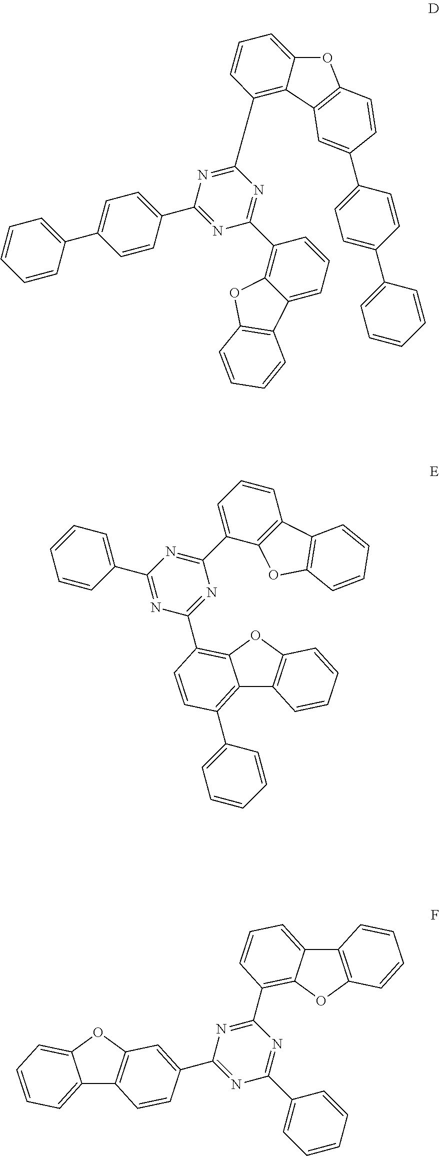 [SMILES: c1ccc(-c2ccc(-c3ccc4oc5cccc(-c6nc(-c7ccc(-c8ccccc8)cc7)nc(-c7cccc8c7oc7ccccc78)n6)c5c4c3)cc2)cc1 ; c1ccc(-c2nc(-c3cccc4c3oc3ccccc34)nc(-c3ccc(-c4ccccc4)c4c3oc3ccccc34)n2)cc1 ; c1ccc(-c2nc(-c3ccc4c(c3)oc3ccccc34)nc(-c3cccc4c3oc3ccccc34)n2)cc1]

TABLE 10

| | | Driving | Effi- | Color | Service |
|---|---|---|---|---|---|
| | | voltage | ciency | coordinate | life |
| | Compound | (V) | (cd/A) | (x, y) | (T₉₀) |
| Comparative | A | 5.72 | 46.7 | (0.273, 0.684) | 55 |
| Example 1 | | | | | |
| Comparative | B | 5.55 | 50.2 | (0.277, 0.674) | 65 |
| Example 2 | | | | | |
| Comparative | C | 5.23 | 59.2 | (0.271, 0.686) | 43 |
| Example 3 | | | | | |
| Comparative | D | 5.33 | 58.2 | (0.274, 0.676) | 53 |
| Example 4 | | | | | |
| Comparative | E | 6.32 | 66.2 | (0.279, 0.677) | 65 |
| Example 5 | | | | | |
| Comparative | F | 6.88 | 61.7 | (0.278, 0.677) | 56 |
| Example 6 | | | | | |
| Example 1 | 1-1 | 4.01 | 80.2 | (0.277, 0.669) | 154 |
| Example 2 | 1-2 | 3.72 | 89.3 | (0.281, 0.679) | 159 |
| Example 3 | 1-7 | 3.76 | 85.2 | (0.280, 0.677) | 168 |
| Example 4 | 1-11 | 3.98 | 81.7 | (0.279, 0.676) | 154 |
| Example 5 | 1-12 | 4.64 | 77.2 | (0.272, 0.669) | 148 |
| Example 6 | 1-13 | 4.32 | 76.4 | (0.271, 0.671) | 158 |
| Example 7 | 1-18 | 3.88 | 87.2 | (0.275, 0.672) | 158 |
| Example 8 | 1-20 | 3.74 | 86.3 | (0.279, 0.675) | 162 |
| Example 9 | 1-28 | 3.84 | 80.4 | (0.280, 0.676) | 150 |
| Example 10 | 1-29 | 3.72 | 82.6 | (0.278, 0.672) | 149 |
| Example 11 | 1-33 | 4.53 | 75.4 | (0.283, 0.687) | 144 |
| Example 12 | 1-35 | 4.32 | 70.2 | (0.286, 0.686) | 143 |
| Example 13 | 1-37 | 4.42 | 76.4 | (0.274, 0.678) | 151 |
| Example 14 | 1-45 | 3.99 | 85.2 | (0.279, 0.677) | 169 |
| Example 15 | 1-46 | 3.88 | 84.2 | (0.278, 0.677) | 160 |
| Example 16 | 1-47 | 4.02 | 72.3 | (0.276, 0.671) | 152 |
| Example 17 | 1-51 | 4.46 | 76.4 | (0.284, 0.687) | 149 |
| Example 18 | 1-53 | 4.51 | 78.4 | (0.281, 0.684) | 153 |
| Example 19 | 1-57 | 4.21 | 80.2 | (0.283, 0.681) | 146 |
| Example 20 | 1-59 | 4.11 | 77.5 | (0.279, 0.681) | 146 |
| Example 21 | 1-65 | 4.08 | 78.7 | (0.280, 0.679) | 132 |
| Example 22 | 1-66 | 4.32 | 80.2 | (0.281, 0.678) | 137 |
| Example 23 | 1-68 | 3.86 | 78.2 | (0.278, 0.682) | 141 |
| Example 24 | 1-69 | 4.01 | 70.3 | (0.286, 0.692) | 132 |
| Example 25 | 1-70 | 3.85 | 78.2 | (0.274, 0.672) | 138 |
| Example 26 | 1-75 | 3.71 | 80.4 | (0.278, 0.678) | 139 |
| Example 27 | 1-78 | 4.11 | 78.4 | (0.277, 0.674) | 151 |
| Example 28 | 1-79 | 4.32 | 72.2 | (0.279, 0.674) | 143 |
| Example 29 | 1-83 | 4.23 | 79.3 | (0.278, 0.677) | 153 |
| Example 30 | 1-93 | 4.31 | 78.2 | (0.283, 0.677) | 141 |
| Example 31 | 1-94 | 4.18 | 86.4 | (0.273, 0.678) | 149 |
| Example 32 | 1-95 | 4.23 | 82.3 | (0.275, 0.672) | 151 |
| Example 33 | 1-101 | 4.31 | 86.4 | (0.279, 0.675) | 169 |
| Example 34 | 1-109 | 4.18 | 78.4 | (0.280, 0.676) | 160 |
| Example 35 | 1-111 | 4.08 | 80.2 | (0.278, 0.672) | 152 |
| Example 36 | 1-112 | 4.32 | 77.5 | (0.283, 0.687) | 149 |
| Example 37 | 1-115 | 3.86 | 88.7 | (0.286, 0.686) | 153 |
| Example 38 | 1-117 | 4.01 | 80.2 | (0.274, 0.678) | 146 |
| Example 39 | 1-118 | 3.85 | 78.2 | (0.279, 0.677) | 146 |
| Example 40 | 1-120 | 3.71 | 80.3 | (0.278, 0.677) | 132 |
| Example 41 | 1-123 | 4.31 | 78.2 | (0.276, 0.671) | 137 |
| Example 42 | 1-124 | 4.18 | 80.4 | (0.283, 0.687) | 141 |
| Example 43 | 1-135 | 4.23 | 88.4 | (0.286, 0.686) | 132 |
| Example 44 | 1-138 | 4.31 | 82.2 | (0.274, 0.678) | 158 |
| Example 45 | 1-145 | 4.18 | 82.3 | (0.279, 0.677) | 162 |
| Example 46 | 1-149 | 4.08 | 76.4 | (0.278, 0.677) | 150 |
| Example 47 | 1-151 | 4.32 | 78.4 | (0.276, 0.671) | 149 |
| Example 48 | 1-155 | 3.86 | 80.2 | (0.283, 0.687) | 144 |
| Example 49 | 1-160 | 4.46 | 87.5 | (0.286, 0.686) | 143 |
| Example 50 | 1-161 | 4.51 | 78.7 | (0.274, 0.678) | 151 |
| Example 51 | 1-166 | 4.21 | 80.2 | (0.279, 0.677) | 169 |
| Example 52 | 1-170 | 4.11 | 78.2 | (0.278, 0.677) | 160 |
| Example 53 | 1-173 | 4.08 | 80.3 | (0.276, 0.671) | 152 |
| Example 54 | 1-177 | 4.32 | 88.2 | (0.283, 0.687) | 149 |
| Example 55 | 1-180 | 3.86 | 80.4 | (0.286, 0.686) | 143 |
| Example 56 | 1-181 | 3.56 | 90.2 | (0.276, 0.671) | 172 |
| Example 57 | 1-182 | 3.66 | 89.3 | (0.283, 0.687) | 170 |
| Example 58 | 1-188 | 3.54 | 91.2 | (0.286, 0.686) | 173 |
| Example 59 | 1-191 | 3.57 | 90.7 | (0.274, 0.678) | 172 |
| Example 60 | 1-192 | 3.67 | 89.9 | (0.279, 0.677) | 169 |
| Example 61 | 1-199 | 3.59 | 90.3 | (0.278, 0.677) | 171 |
| Example 62 | 1-201 | 3.56 | 92.0 | (0.276, 0.671) | 172 |
| Example 63 | 1-213 | 3.58 | 91.1 | (0.283, 0.687) | 172 |
| Example 64 | 1-214 | 3.69 | 89.1 | (0.286, 0.686) | 169 |
| Example 65 | 1-219 | 3.59 | 91.3 | (0.278, 0.677) | 174 |

It can be seen that the heterocyclic compound of the present invention has excellent thermal stability and light emitting efficiency by having an aryl group as a substituent of dibenzofuran or dibenzothiophene linked to triazine.

When the heteroaryl group is substituted at a different position (the heteroaryl group is substituted at a different position in all the example compounds) rather than at the same position (Comparative Example 1) in the triazine, the compound is sterically hindered, so that since the thermal stability is increased, the durability and stability of the device are improved, and thus, there is an effect that the service life of the device is improved.

When the number of carbon atoms in the aryl group substitution of the heterocyclic moiety bonded to the triazine is 12 or more (Comparative Examples 3 and 4), the triplet energy level is lower than that of the compound of the present invention, so that there is a disadvantage in that the deposition temperature is increased when the organic material layer of the device is prepared. That is, when a compound substituted with a substituent having 12 or less carbon atoms, such as the compound according to the present application, is used in an organic light emitting device, excellent characteristics in driving, efficiency and service life of the device are exhibited.

In the case of the compound of the present invention, it can be seen that the driving voltage, efficiency and service life are excellent because the HOMO level is delocalized over a heterocyclic moiety in which the aryl group is substituted to effectively stabilize holes, the LUMO level is delocalized over a heterocyclic moiety (a moiety in which the aryl group is not substituted) bonded to the triazine to effectively stabilizes electrons, and the compound has a molecular weight and a bandgap suitable for use in the light emitting layer.

In addition, it can be seen that compounds whose HOMO level is deuterated have a better ability to inject holes than compounds whose HOMO level is not deuterated (compounds with total deuterium substitution of Examples 57, 60 and 64), and thus exhibit a better effect during a device evaluation.

### <Experimental Example 2> Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

A hole injection layer 4,4′,4″-tris[2-naphthyl(phenyl)amino]triphenylamine (2-TNATA) and a hole transport layer N,N′-di(1-naphthyl)-N,N′-diphenyl-(1,1′-biphenyl)-4,4′-diamine (NPB), which are common layers, were formed on the ITO transparent electrode (positive electrode).

A light emitting layer was thermally vacuum deposited thereon as follows. The light emitting layer was deposited to have a thickness of 360 Å by pre-mixing the compound described in the following Table 11, and then depositing the compound in one common source, using the compound as a host and tris(2-phenylpyridine)iridium (Ir(ppy)₃) as a green phosphorescent dopant, and doping the host with Ir(ppy)₃ in an amount of 7%.

Thereafter, BCP as a hole blocking layer was deposited to have a thickness of 60 Å, and Alq₃ as an electron transport layer was deposited to have a thickness of 200 Å thereon. Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1200 Å on the electron injection layer to form a negative electrode.

Meanwhile, all the organic compounds required for manufacturing an OLED device were subjected to vacuum sublimed purification under 10⁻⁶ to 10⁻⁸ torr for each material, and used for the manufacture of OLED.

The organic light emitting devices of Comparative Examples 7 to 19 were manufactured in the same manner as in Experimental Example 2, except that the compounds in the following Table 11 were used as the host of the light emitting layer.

For the organic light emitting device manufactured as described above, electroluminescence (EL) characteristics were measured by M7000 manufactured by McScience Inc., and based on the measurement result thereof, T90 was measured by a service life measurement device (M6000) manufactured by McScience Inc., when the reference luminance was 6,000 cd/m². The results of measuring the driving voltage, light emitting efficiency, color coordinate (CIE) and service life of the organic light emitting device manufactured according to the present invention are shown in the following Table 11.

TABLE 11

| | Light | | | | | |
|---|---|---|---|---|---|---|
| | emitting | Ratio | Driving | | Color | Service |
| | layer | (Weight | voltage | Efficiency | coordinate | life |
| | Compound | ratio) | (V) | (cd/A) | (x, y) | (T₉₀) |
| Comparative | A: 2-82 | 1:3 | 4.69 | 110.0 | (0.277, 0.682) | 209 |
| Example 7 | | | | | | |
| Comparative | | 1:2 | 4.62 | 111.1 | (0.277, 0.678) | 203 |
| Example 8 | | | | | | |
| Comparative | B: 2-82 | 1:3 | 4.67 | 101.8 | (0.276, 0.679) | 206 |
| Example 9 | | | | | | |
| Comparative | | 1:2 | 4.64 | 109.2 | (0.277, 0.677) | 206 |
| Example 10 | | | | | | |
| Comparative | C: 2-85 | 1:3.5 | 4.69 | 103.7 | (0.277, 0.680) | 209 |
| Example 11 | | | | | | |
| Comparative | | 1:2.5 | 4.69 | 104.8 | (0.278, 0.681) | 200 |
| Example 12 | | | | | | |
| Comparative | | 1:2 | 4.68 | 104.8 | (0.278, 0.681) | 201 |
| Example 13 | | | | | | |
| Comparative | D: 2-86 | 1:3 | 4.66 | 102.3 | (0.277, 0.682) | 200 |
| Example 14 | | | | | | |
| Comparative | | 1:2 | 4.62 | 105.2 | (0.278, 0.683) | 209 |
| Example 15 | | | | | | |
| Comparative | E: 2-13 | 1:3 | 5.66 | 101.3 | (0.277, 0.682) | 192 |
| Example 16 | | | | | | |
| Comparative | | 1:2 | 5.60 | 103.2 | (0.278, 0.683) | 198 |
| Example 17 | | | | | | |
| Comparative | F: 2-82 | 1:3 | 4.86 | 100.3 | (0.277, 0.682) | 201 |
| Example 18 | | | | | | |
| Comparative | | 1:2 | 4.72 | 102.2 | (0.278, 0.683) | 199 |
| Example 19 | | | | | | |
| Example 66 | 1-1: 2-81 | 1:3.5 | 3.75 | 176.6 | (0.277, 0.678) | 310 |
| Example 67 | | 1:3 | 3.70 | 178.4 | (0.277, 0.682) | 308 |
| Example 68 | | 1:2 | 3.68 | 178.8 | (0.277, 0.678) | 304 |
| Example 69 | | 1:1 | 3.66 | 179.2 | (0.276, 0.679) | 303 |
| Example 70 | 1-2: 2-82 | 1:3 | 3.68 | 172.3 | (0.277, 0.677) | 307 |
| Example 71 | | 1:2 | 3.60 | 178.8 | (0.277, 0.680) | 306 |
| Example 72 | 1-3: 2-86 | 1:3 | 3.66 | 173.4 | (0.278, 0.681) | 304 |
| Example 73 | | 1:2 | 3.62 | 179.6 | (0.277, 0.682) | 300 |
| Example 74 | 1-7: 2-87 | 1:2.5 | 3.66 | 172.2 | (0.278, 0.683) | 310 |
| Example 75 | | 1:1 | 3.60 | 177.3 | (0.277, 0.682) | 309 |
| Example 76 | 1-10: 2-83 | 1:3.5 | 3.70 | 171.4 | (0.277, 0.678) | 310 |
| Example 77 | | 1:2 | 3.66 | 173.2 | (0.276, 0.679) | 306 |
| Example 78 | 1-15: 2-88 | 1:3 | 3.71 | 172.3 | (0.277, 0.677) | 309 |
| Example 79 | | 1:2 | 3.68 | 174.6 | (0.277, 0.680) | 304 |
| Example 80 | | 1:1 | 3.64 | 177.9 | (0.278, 0.683) | 303 |
| Example 81 | 1-20: 2-80 | 1:3 | 3.66 | 174.3 | (0.277, 0.682) | 310 |
| Example 82 | | 1:2 | 3.64 | 176.7 | (0.277, 0.678) | 306 |
| Example 83 | | 1:1 | 3.60 | 179.4 | (0.276, 0.679) | 305 |
| Example 84 | 1-29: 2-87 | 1:3 | 3.60 | 186.2 | (0.277, 0.677) | 310 |
| Example 85 | | 1:2 | 3.59 | 187.3 | (0.277, 0.680) | 308 |
| Example 86 | 1-30: 2-88 | 1:3 | 3.60 | 182.7 | (0.278, 0.681) | 307 |
| Example 87 | | 1:2 | 3.59 | 186.3 | (0.278, 0.683) | 304 |
| Example 88 | 1-31: 2-87 | 1:3 | 3.68 | 176.3 | (0.277, 0.682) | 288 |
| Example 89 | | 1:2 | 3.66 | 180.1 | (0.277, 0.678) | 285 |
| Example 90 | 1-35: 2-86 | 1:3 | 3.48 | 190.7 | (0.281, 0.676) | 375 |
| Example 91 | | 1:2 | 3.40 | 194.4 | (0.280, 0.676) | 373 |
| Example 92 | 1-36: 2-81 | 1:3 | 3.66 | 178.2 | (0.276, 0.679) | 292 |
| Example 93 | | 1:2 | 3.65 | 176.6 | (0.275, 0.679) | 290 |
| Example 94 | 1-37: 2-85 | 1:3 | 3.42 | 194.2 | (0.278, 0.678) | 377 |
| Example 95 | | 1:2 | 3.39 | 196.3 | (0.279, 0.677) | 376 |
| Example 96 | 1-45: 2-4 | 1:3 | 3.98 | 148.2 | (0.275, 0.680) | 235 |
| Example 97 | | 1:2 | 3.92 | 149.7 | (0.274, 0.678) | 230 |
| Example 98 | 1-45: 2-27 | 1:3 | 3.90 | 148.9 | (0.276, 0.676) | 250 |
| Example 99 | | 1:2 | 3.85 | 150.0 | (0.274, 0.679) | 248 |
| Example 100 | 1-45: 2-32 | 1:3 | 3.91 | 147.7 | (0.288, 0.681) | 250 |
| Example 101 | | 1:2 | 3.87 | 148.9 | (0.285, 0.680) | 249 |
| Example 102 | 1-45: 2-33 | 1:3 | 3.88 | 148.8 | (0.276, 0.679) | 248 |
| Example 103 | | 1:2 | 3.85 | 149.2 | (0.276, 0.676) | 246 |
| Example 104 | 1-45: 2-42 | 1:3 | 3.92 | 152.6 | (0.278, 0.681) | 210 |
| Example 105 | | 1:2.5 | 3.92 | 158.4 | (0.275, 0.680) | 207 |
| Example 106 | | 1:2 | 3.90 | 150.3 | (0.274, 0.679) | 205 |
| Example 107 | | 1:1 | 3.88 | 151.1 | (0.288, 0.681) | 203 |
| Example 108 | 1-45: 2-45 | 1:3 | 3.96 | 159.4 | (0.285, 0.680) | 230 |
| Example 109 | | 1:2 | 3.94 | 151.1 | (0.276, 0.679) | 226 |
| Example 110 | | 1:1.5 | 3.85 | 156.3 | (0.276, 0.676) | 220 |
| Example 111 | | 1:1 | 3.85 | 157.6 | (0.278, 0.681) | 200 |
| Example 112 | 1-45: 2-65 | 1:3 | 3.80 | 163.7 | (0.274, 0.679) | 300 |
| Example 113 | | 1:2 | 3.78 | 165.5 | (0.288, 0.681) | 296 |
| Example 114 | | 1:1.5 | 3.77 | 166.7 | (0.285, 0.680) | 295 |
| Example 115 | | 1:1 | 3.74 | 168.9 | (0.276, 0.679) | 290 |
| Example 116 | 1-46: 2-69 | 1:3 | 3.78 | 164.2 | (0.274, 0.679) | 298 |
| Example 117 | | 1:2.5 | 3.76 | 166.4 | (0.288, 0.681) | 295 |
| Example 118 | | 1:2 | 3.75 | 167.9 | (0.285, 0.680) | 290 |
| Example 119 | 1-47: 2-74 | 1:2 | 3.79 | 167.3 | (0.276, 0.679) | 292 |
| Example 120 | | 1:1.5 | 3.76 | 169.2 | (0.280, 0.676) | 290 |
| Example 121 | | 1:1 | 3.75 | 170.0 | (0.276, 0.679) | 288 |
| Example 122 | 1-47: 2-82 | 1:3.5 | 3.74 | 171.1 | (0.274, 0.678) | 310 |
| Example 123 | | 1:2.5 | 3.72 | 175.6 | (0.276, 0.676) | 308 |
| Example 124 | | 1:2 | 3.63 | 177.3 | (0.274, 0.679) | 305 |
| Example 125 | | 1:1 | 3.60 | 180.4 | (0.288, 0.681) | 304 |
| Example 126 | 1-47: 2-83 | 1:3 | 3.62 | 173.2 | (0.280, 0.676) | 309 |
| Example 127 | | 1:2 | 3.61 | 178.3 | (0.276, 0.679) | 308 |
| Example 128 | 1-51: 2-85 | 1:3 | 3.70 | 178.3 | (0.274, 0.679) | 312 |
| Example 129 | | 1:2 | 3.68 | 180.7 | (0.288, 0.681) | 309 |
| Example 130 | | 1:1 | 3.65 | 181.3 | (0.285, 0.680) | 306 |
| Example 131 | 1-45: 2-88 | 1:3 | 3.69 | 178.7 | (0.275, 0.680) | 310 |
| Example 132 | | 1:2.5 | 3.62 | 180.3 | (0.274, 0.679) | 309 |
| Example 133 | | 1:1 | 3.61 | 181.1 | (0.274, 0.679) | 306 |
| Example 134 | 1-42: 2-83 | 1:3.5 | 3.75 | 173.2 | (0.277, 0.678) | 310 |
| Example 135 | | 1:2.5 | 3.70 | 178.3 | (0.276, 0.679) | 309 |
| Example 136 | | 1:2 | 3.66 | 180.4 | (0.277, 0.682) | 304 |
| Example 137 | 1-44: 2-85 | 1:3 | 3.74 | 171.3 | (0.277, 0.678) | 308 |
| Example 138 | | 1:2 | 3.70 | 176.1 | (0.276, 0.677) | 304 |
| Example 139 | | 1:1.5 | 3.68 | 179.4 | (0.280, 0.679) | 303 |
| Example 140 | 1-59: 2-87 | 1:2 | 3.75 | 170.6 | (0.278, 0.681) | 310 |
| Example 141 | | 1:1 | 3.65 | 179.3 | (0.276, 0.679) | 309 |
| Example 142 | 1-59: 2-98 | 1:3 | 3.70 | 170.4 | (0.277, 0.678) | 310 |
| Example 143 | | 1:2 | 3.62 | 177.7 | (0.276, 0.677) | 305 |
| Example 144 | 1-65: 2-96 | 1:3 | 3.77 | 170.6 | (0.280, 0.679) | 308 |
| Example 145 | | 1:2 | 3.65 | 174.8 | (0.278, 0.681) | 306 |
| Example 146 | 1-66: 2-88 | 1:3 | 3.71 | 170.3 | (0.276, 0.677) | 310 |
| Example 147 | | 1:2.5 | 3.68 | 173.7 | (0.280, 0.679) | 308 |
| Example 148 | | 1:2 | 3.65 | 175.4 | (0.278, 0.681) | 307 |
| Example 149 | 1-69: 2-89 | 1:3.5 | 3.68 | 170.7 | (0.277, 0.678) | 308 |
| Example 150 | | 1:2.5 | 3.62 | 172.7. | (0.276, 0.677) | 305 |
| Example 151 | | 1:2 | 3.60 | 173.7 | (0.280, 0.679) | 304 |
| Example 152 | 1-70: 2-80 | 1:3.5 | 3.73 | 170.3 | (0.276, 0.677) | 310 |
| Example 153 | | 1:2 | 3.70 | 172.6 | (0.278, 0.681) | 304 |
| Example 154 | | 1:1 | 3.65 | 175.3 | (0.276, 0.679) | 302 |
| Example 155 | 1-75: 2-86 | 1:3 | 3.70 | 171.1 | (0.276, 0.677) | 305 |
| Example 156 | | 1:2.5 | 3.64 | 176.3 | (0.280, 0.679) | 303 |
| Example 157 | 1-57: 2-88 | 1:3.5 | 3.71 | 170.4 | (0.277, 0.678) | 310 |
| Example 158 | | 1:2 | 3.68 | 173.1 | (0.276, 0.677) | 306 |
| Example 159 | | 1:1 | 3.62 | 174.3 | (0.280, 0.679) | 303 |
| Example 160 | 1-69: 2-82 | 1:3.5 | 3.60 | 182.3 | (0.276, 0.677) | 310 |
| Example 161 | | 1:2.5 | 3.59 | 188.3 | (0.280, 0.679) | 309 |
| Example 162 | | 1:2 | 3.56 | 190.1 | (0.278, 0.681) | 308 |
| Example 163 | | 1:1 | 3.55 | 190.7 | (0.278, 0.681) | 303 |
| Example 164 | 1-71: 2-82 | 1:3.5 | 3.70 | 174.2 | (0.276, 0.677) | 300 |
| Example 165 | | 1:2.5 | 3.66 | 177.9 | (0.280, 0.679) | 299 |
| Example 166 | | 1:2 | 3.65 | 179.2 | (0.278, 0.681) | 294 |
| Example 167 | | 1:1 | 3.60 | 180.3 | (0.276, 0.677) | 290 |
| Example 168 | 1-75: 2-16 | 1:3 | 3.62 | 176.6 | (0.280, 0.679) | 300 |
| Example 169 | | 1:2 | 3.60 | 178.4 | (0.276, 0.677) | 292 |
| Example 170 | 1-75: 2-33 | 1:3 | 3.61 | 177.9 | (0.278, 0.681) | 299 |
| Example 171 | | 1:2 | 3.60 | 179.9 | (0.276, 0.679) | 296 |
| Example 172 | 1-75: 2-42 | 1:3.5 | 3.61 | 183.2 | (0.276, 0.677) | 320 |
| Example 173 | | 1:3 | 3.56 | 185.1 | (0.280, 0.679) | 315 |
| Example 174 | | 1:2 | 3.55 | 185.5 | (0.277, 0.678) | 314 |
| Example 175 | 1-75: 2-66 | 1:3 | 3.53 | 187.3 | (0.276, 0.677) | 340 |
| Example 176 | | 1:2 | 3.50 | 189.2 | (0.280, 0.679) | 338 |
| Example 177 | 1-75: 2-74 | 1:3 | 3.52 | 188.3 | (0.280, 0.679) | 339 |
| Example 178 | | 1:2 | 3.50 | 190.1 | (0.276, 0.677) | 336 |
| Example 179 | 1-75: 2-82 | 1:3.5 | 3.47 | 191.3 | (0.277, 0.678) | 380 |
| Example 180 | | 1:2.5 | 3.45 | 194.7 | (0.276, 0.677) | 378 |
| Example 181 | | 1:2 | 3.41 | 199.3 | (0.280, 0.679) | 377 |
| Example 182 | | 1:1 | 3.39 | 200.7 | (0.280, 0.679) | 370 |
| Example 183 | 1-77: 2-82 | 1:3.5 | 3.60 | 188.3 | (0.274, 0.679) | 311 |
| Example 184 | | 1:2.5 | 3.59 | 189.7 | (0.288, 0.681) | 308 |
| Example 185 | | 1:2 | 3.57 | 190.3 | (0.285, 0.680) | 307 |
| Example 186 | | 1:1 | 3.55 | 192.3 | (0.285, 0.680) | 304 |
| Example 187 | 1-83: 2-82 | 1:3.5 | 3.50 | 194.4 | (0.276, 0.677) | 380 |
| Example 188 | | 1:3 | 3.48 | 197.2 | (0.278, 0.681) | 374 |
| Example 189 | | 1:2.5 | 3.45 | 198.3 | (0.276, 0.679) | 370 |
| Example 190 | | 1:2 | 3.40 | 199.0 | (0.274, 0.679) | 369 |
| Example 191 | | 1:1 | 3.30 | 199.8 | (0.288, 0.681) | 368 |
| Example 192 | 1-83: 2-81 | 1:3 | 3.66 | 178.3 | (0.277, 0.678) | 310 |
| Example 193 | | 1:2 | 3.64 | 180.2 | (0.276, 0.677) | 308 |
| Example 194 | 1-83: 2-84 | 1:2.5 | 3.63 | 177.6 | (0.280, 0.679) | 309 |
| Example 195 | | 1:2 | 3.62 | 178.2 | (0.280, 0.679) | 308 |
| Example 196 | | 1:1 | 3.60 | 179.3 | (0.274, 0.679) | 307 |
| Example 197 | 1-9032-82 | 1:3 | 3.70 | 176.3 | (0.288, 0.681) | 310 |
| Example 198 | | 1:2.5 | 3.66 | 178.9 | (0.285, 0.680) | 309 |
| Example 199 | | 1:1 | 3.62 | 180.2 | (0.285, 0.680) | 308 |
| Example 200 | 1-94: 2-81 | 1:3 | 3.68 | 176.3 | (0.277, 0.678) | 309 |
| Example 201 | | 1:2 | 3.64 | 177.7 | (0.276, 0.677) | 304 |
| Example 202 | 1-95: 2-83 | 1:2.5 | 3.69 | 177.7 | (0.280, 0.679) | 309 |
| Example 203 | | 1:2 | 3.64 | 179.3 | (0.280, 0.679) | 308 |
| Example 204 | | 1:1 | 3.60 | 180.4 | (0.274, 0.679) | 306 |
| Example 205 | 1-101: 2-84 | 1:3 | 3.62 | 174.5 | (0.288, 0.681) | 309 |
| Example 206 | | 1:2 | 3.60 | 177.7 | (0.285, 0.680) | 304 |
| Example 207 | 1-109: 2-81 | 1:3 | 3.58 | 188.7 | (0.285, 0.680) | 310 |
| Example 208 | | 1:2 | 3.56 | 190.2 | (0.277, 0.678) | 309 |
| Example 209 | 1-115: 2-83 | 1:3 | 3.42 | 194.9 | (0.276, 0.677) | 380 |
| Example 210 | | 1:2 | 3.40 | 199.2 | (0.280, 0.679) | 377 |
| Example 211 | 1-120: 2-91 | 1:3 | 3.40 | 196.3 | (0.280, 0.679) | 379 |
| Example 212 | | 1:2 | 3.32 | 199.4 | (0.274, 0.679) | 377 |
| Example 213 | 1-124: 2-97 | 1:3 | 3.70 | 178.4 | (0.288, 0.681) | 310 |
| Example 214 | | 1:2 | 3.68 | 179.9 | (0.285, 0.680) | 308 |
| Example 215 | 1-138: 2-91 | 1:3 | 3.68 | 176.4 | (0.285, 0.680) | 309 |
| Example 216 | | 1:2.5 | 3.62 | 177.2 | (0.277, 0.678) | 307 |
| Example 217 | | 1:2 | 3.60 | 179.5 | (0.276, 0.677) | 306 |
| Example 218 | 1-145: 2-91 | 1:3 | 3.64 | 178.4 | (0.280, 0.679) | 308 |
| Example 219 | | 1:2.5 | 3.62 | 179.0 | (0.280, 0.679) | 307 |
| Example 220 | | 1:2 | 3.60 | 170.1 | (0.274, 0.679) | 306 |
| Example 221 | 1-149: 2-88 | 1:3 | 3.60 | 181.4 | (0.288, 0.681) | 310 |
| Example 222 | | 1:2 | 3.59 | 188.7 | (0.285, 0.680) | 308 |
| Example 223 | 1-153: 2-97 | 1:3 | 3.64 | 179.2 | (0.285, 0.680) | 304 |
| Example 224 | | 1:2 | 3.62 | 180.8 | (0.277, 0.678) | 300 |
| Example 225 | 1-166: 2-82 | 1:3 | 3.70 | 178.4 | (0.276, 0.677) | 310 |
| Example 226 | | 1:2 | 3.68 | 179.5 | (0.280, 0.679) | 308 |
| Example 227 | 1-176: 2-88 | 1:3 | 3.40 | 192.9 | (0.280, 0.679) | 380 |
| Example 228 | | 1:2 | 3.38 | 199.4 | (0.274, 0.679) | 378 |
| Example 229 | 1-180: 2-85 | 1:3 | 3.38 | 197.4 | (0.288, 0.681) | 379 |
| Example 230 | | 1:2 | 3.34 | 199.6 | (0.285, 0.680) | 375 |
| Example 231 | 1-162: 2-4 | 1:3 | 3.62 | 174.5 | (0.288, 0.681) | 309 |
| Example 232 | | 1:2 | 3.60 | 177.7 | (0.285, 0.680) | 304 |
| Example 233 | 1-162: 2-27 | 1:3 | 3.70 | 176.3 | (0.288, 0.681) | 300 |
| Example 234 | | 1:2.5 | 3.66 | 178.9 | (0.285, 0.680) | 299 |
| Example 235 | | 1:1 | 3.62 | 180.2 | (0.285, 0.680) | 298 |
| Example 236 | 1-163: 2-27 | 1:3 | 3.66 | 178.3 | (0.277, 0.678) | 310 |
| Example 237 | | 1:2 | 3.64 | 179.2 | (0.276, 0.677) | 309 |
| Example 238 | 1-169: 2-33 | 1:3 | 3.80 | 163.7 | (0.274, 0.679) | 300 |
| Example 239 | | 1:2 | 3.78 | 165.5 | (0.288, 0.681) | 296 |
| Example 240 | 1-169: 2-40 | 1:3 | 3.62 | 174.5 | (0.288, 0.681) | 300 |
| Example 241 | | 1:2 | 3.60 | 177.7 | (0.285, 0.680) | 292 |
| Example 242 | 1-169: 2-38 | 1:2.5 | 3.66 | 177.9 | (0.280, 0.679) | 299 |
| Example 243 | | 1:2 | 3.65 | 179.2 | (0.278, 0.681) | 294 |
| Example 244 | 1-170: 2-6 | 1:3 | 3.60 | 181.4 | (0.288, 0.681) | 310 |
| Example 245 | | 1:2 | 3.59 | 188.7 | (0.285, 0.680) | 308 |
| Example 246 | 1-170: 2-27 | 1:3 | 3.62 | 182.5 | (0.288, 0.681) | 303 |
| Example 247 | | 1:2 | 3.60 | 180.7 | (0.285, 0.680) | 300 |
| Example 248 | 1-170: 2-32 | 1:3 | 3.48 | 180.7 | (0.281, 0.676) | 300 |
| Example 249 | | 1:2 | 3.40 | 179.4 | (0.280, 0.676) | 299 |
| Example 250 | 1-175: 2-20 | 1:3 | 3.66 | 178.2 | (0.276, 0.679) | 301 |
| Example 251 | | 1:2 | 3.65 | 176.6 | (0.275, 0.679) | 200 |
| Example 252 | 1-175: 2-24 | 1:3 | 3.42 | 174.2 | (0.278, 0.678) | 307 |
| Example 253 | | 1:2 | 3.39 | 176.3 | (0.279, 0.677) | 306 |
| Example 254 | 1-179: 2-40 | 1:3 | 3.98 | 180.2 | (0.275, 0.680) | 305 |
| Example 255 | | 1:2 | 3.92 | 179.7 | (0.274, 0.678) | 300 |
| Example 256 | 1-181: 2-3 | 1:3 | 3.36 | 198.2 | (0.276, 0.679) | 371 |
| Example 257 | | 1:2 | 3.35 | 196.6 | (0.275, 0.679) | 360 |
| Example 258 | 1-181: 2-23 | 1:3 | 3.39 | 194.2 | (0.278, 0.678) | 375 |
| Example 259 | | 1:2 | 3.35 | 192.3 | (0.279, 0.677) | 372 |
| Example 260 | 1-182: 2-86 | 1:3 | 3.26 | 201.2 | (0.276, 0.679) | 413 |
| Example 261 | | 1:2 | 3.25 | 200.6 | (0.275, 0.679) | 411 |
| Example 262 | 1-182: 2-88 | 1:3 | 3.26 | 202.2 | (0.276, 0.679) | 410 |
| Example 263 | | 1:2 | 3.25 | 200.6 | (0.275, 0.679) | 408 |
| Example 264 | 1-188: 2-64 | 1:3 | 3.39 | 194.2 | (0.278, 0.678) | 375 |
| Example 265 | | 1:2 | 3.35 | 192.3 | (0.279, 0.677) | 372 |
| Example 266 | 1-188: 2-65 | 1:3 | 3.39 | 194.2 | (0.278, 0.678) | 375 |
| Example 267 | | 1:2 | 3.35 | 192.3 | (0.279, 0.677) | 372 |
| Example 268 | 1-188: 2-27 | 1:3 | 3.39 | 194.2 | (0.278, 0.678) | 375 |
| Example 269 | | 1:2.5 | 3.35 | 192.3 | (0.279, 0.677) | 372 |
| Example 270 | | 1:1 | 3.34 | 190.3 | (0.279, 0.677) | 370 |
| Example 271 | 1-213: 2-7 | 1:3 | 3.39 | 194.2 | (0.278, 0.678) | 375 |
| Example 272 | | 1:2 | 3.35 | 192.3 | (0.279, 0.677) | 372 |
| Example 273 | 1-213: 2-80 | 1:3 | 3.29 | 204.2 | (0.278, 0.678) | 415 |
| Example 274 | | 1:2.5 | 3.25 | 202.3 | (0.279, 0.677) | 412 |
| Example 275 | | 1:1 | 3.24 | 202.2 | (0.279, 0.677) | 410 |

When comparing the results in Table 10 with the results in Table 11, it can be confirmed that when both the heterocyclic compound of Chemical Formula 1 and the heterocyclic compound of Chemical Formula 2 are used as hosts of the light emitting layer, all of the driving voltage, the light emitting efficiency and the service life are improved.

From this result, it can be expected that an exciplex phenomenon will occur when both compounds are included. The exciplex phenomenon is a phenomenon in which energy with a magnitude of the HOMO level of a donor (p-host) and the LUMO level of an acceptor (n-host) is released due to an electron exchange between two molecules. When the exciplex phenomenon between two molecules occurs, a reverse intersystem crossing (RISC) occurs, and the internal quantum efficiency of fluorescence can be increased to 100% due to the RISC. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life. In the present invention, it could be confirmed that the compound of Chemical Formula 2 serving as a donor and the compound of Chemical Formula 1 serving as an acceptor exhibit excellent device characteristics when the compounds are used as hosts of the light emitting device.

In contrast, it can be seen that when the compounds (Comparative Examples 7 to 19) out of the scope of the present invention are used in combination with the compound of Chemical Formula 2, the performance in terms of driving voltage, light emitting efficiency and service life deteriorates compared to the present invention.

That is, it can be confirmed that when both the heterocyclic compound of Chemical Formula 1 and the heterocyclic compound of Chemical Formula 2 of the present invention are used as hosts of a light emitting layer, the driving voltage, light emitting efficiency and service life are remarkably excellent.

It could be confirmed that among the compounds represented by Chemical Formulae 1 and 2, the compound including deuterium shows better effects in terms of driving, efficiency, and service life compared to the compound that does not include deuterium, and shows better effects as the content of deuterium is higher.

It could be confirmed that in the case of Chemical Formula 1, when deuterium is included in a heterocyclic compound in which the aryl group at the HOMO level is substituted, the performance of the device is further enhanced compared to compounds with total deuterium substitution, and in the case of Chemical Formula 2, partially deuterated compounds of the aryl group have even better performance than when the deuterium is not substituted, and moreover, the case where the hydrogen of biscarbazole is substituted with deuterium has a better performance of the device, and the higher the content of deuterium is, the better the performance of a device using a higher content of deuterium.

1. A heterocyclic compound represented by the following Chemical Formula 1: 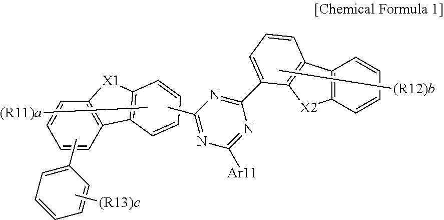
wherein, in Chemical Formula 1,
X1 and X2 are the same as or different from each other, and are each independently O; or S,
Ar11 is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R11 to R13 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
a is an integer from 0 to 6, and when a is 2 or higher, R11's are the same as or different from each other,
b is an integer from 0 to 7, and when b is 2 or higher, R12's are the same as or different from each other, and
c is an integer from 0 to 5, and when c is 2 or higher, R13's are the same as or different from each other.

2. The heterocyclic compound of claim 1, wherein Chemical Formula 1 is represented by any one of the following Chemical Formulae 1-1 to 1-3: 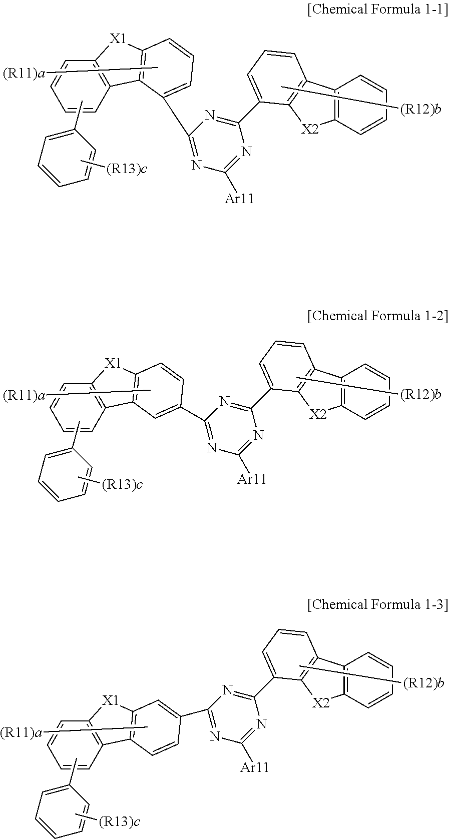
in Chemical Formulae 1-1 to 1-3,
the definitions of X1, X2, Ar11, R11 to R13, a, b and c are the same as the definitions in Chemical Formula 1.

3. The heterocyclic compound of claim 1, wherein Chemical Formula 1 is represented by any one of the following Chemical Formulae 1-A to 1-D: 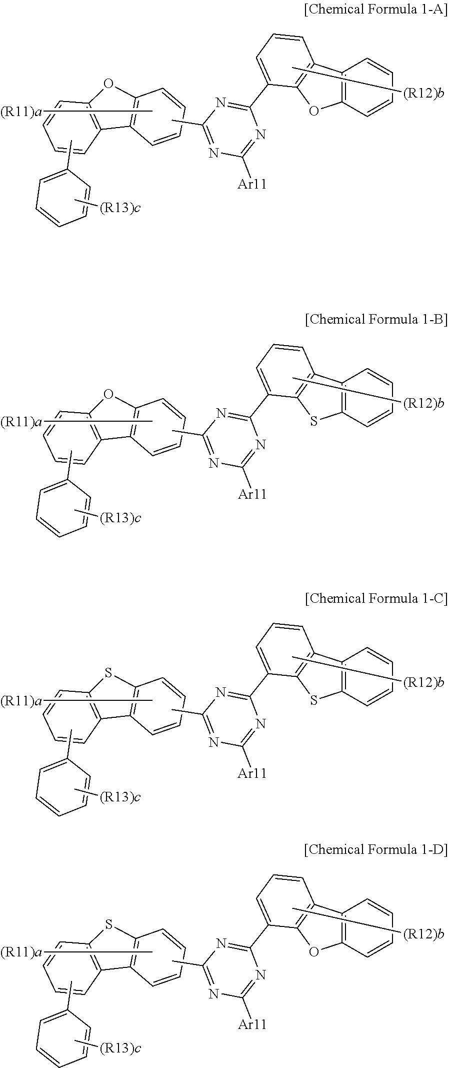
in Chemical Formulae 1-A to 1-D,
the definitions of Ar11, R11 to R13, a, b and c are the same as the definitions in Chemical Formula 1.

4. The heterocyclic compound of claim 1, wherein Ar11 is a substituted or unsubstituted C6 to C20 aryl group.

5. The heterocyclic compound of claim 1, wherein R11 to R13 are the same as or different from each other, and are each independently hydrogen or deuterium.

6. The heterocyclic compound of claim 1, wherein Chemical Formula 1 is dividedly represented by the following Structures 1a to 1c, and a deuterium content of each of the following Structures 1a to 1c is 0% to 100%: 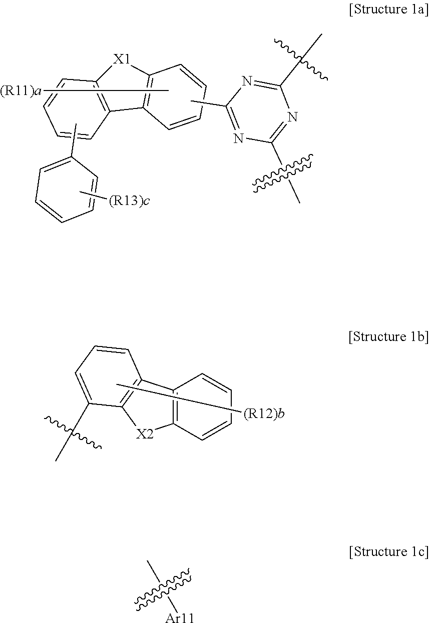
in Structures 1a to 1c,
the definitions of X1, X2, Ar11, R11 to R13, a, b and c are the same as the definitions in Chemical Formula 1, and 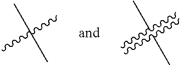
are each a binding position where those which are the same are linked to each other.

7. The heterocyclic compound of claim 1, wherein Chemical Formula 1 is represented by any one of the following compounds:

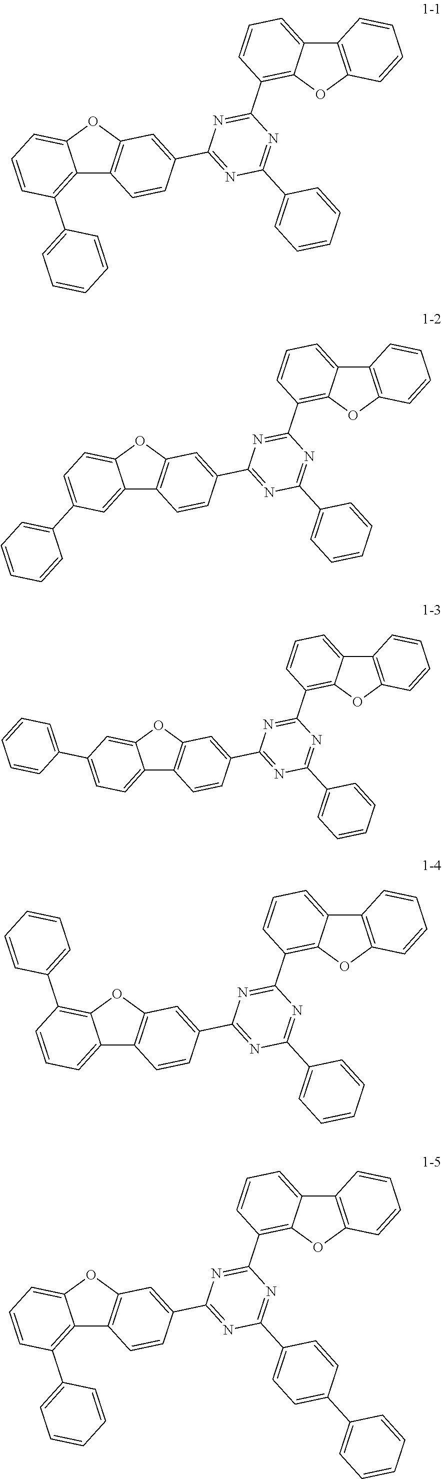 [SMILES: c1ccc(-c2nc(-c3ccc4c(c3)oc3cccc(-c5ccccc5)c34)nc(-c3cccc4c3oc3ccccc34)n2)cc1 ; c1ccc(-c2ccc3oc4cc(-c5nc(-c6ccccc6)nc(-c6cccc7c6oc6ccccc67)n5)ccc4c3c2)cc1 ; c1ccc(-c2ccc3c(c2)oc2cc(-c4nc(-c5ccccc5)nc(-c5cccc6c5oc5ccccc56)n4)ccc23)cc1 ; c1ccc(-c2nc(-c3ccc4c(c3)oc3c(-c5ccccc5)cccc34)nc(-c3cccc4c3oc3ccccc34)n2)cc1 ; c1ccc(-c2ccc(-c3nc(-c4ccc5c(c4)oc4cccc(-c6ccccc6)c45)nc(-c4cccc5c4oc4ccccc45)n3)cc2)cc1]

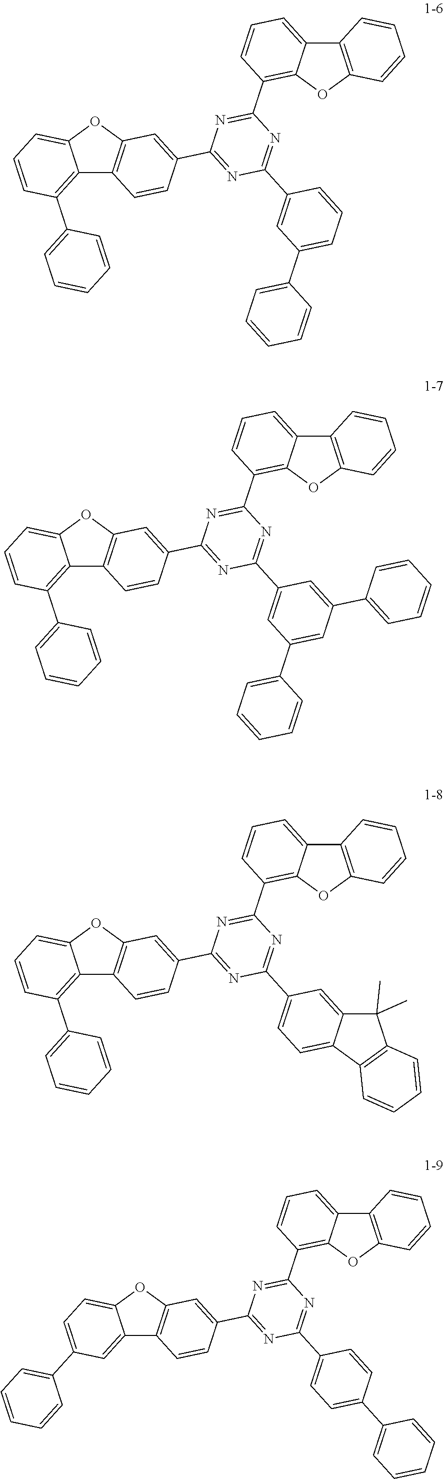 [SMILES: c1ccc(-c2cccc(-c3nc(-c4ccc5c(c4)oc4cccc(-c6ccccc6)c45)nc(-c4cccc5c4oc4ccccc45)n3)c2)cc1 ; c1ccc(-c2cc(-c3ccccc3)cc(-c3nc(-c4ccc5c(c4)oc4cccc(-c6ccccc6)c45)nc(-c4cccc5c4oc4ccccc45)n3)c2)cc1 ; CC1(C)c2ccccc2-c2ccc(-c3nc(-c4ccc5c(c4)oc4cccc(-c6ccccc6)c45)nc(-c4cccc5c4oc4ccccc45)n3)cc21 ; c1ccc(-c2ccc(-c3nc(-c4ccc5c(c4)oc4ccc(-c6ccccc6)cc45)nc(-c4cccc5c4oc4ccccc45)n3)cc2)cc1]

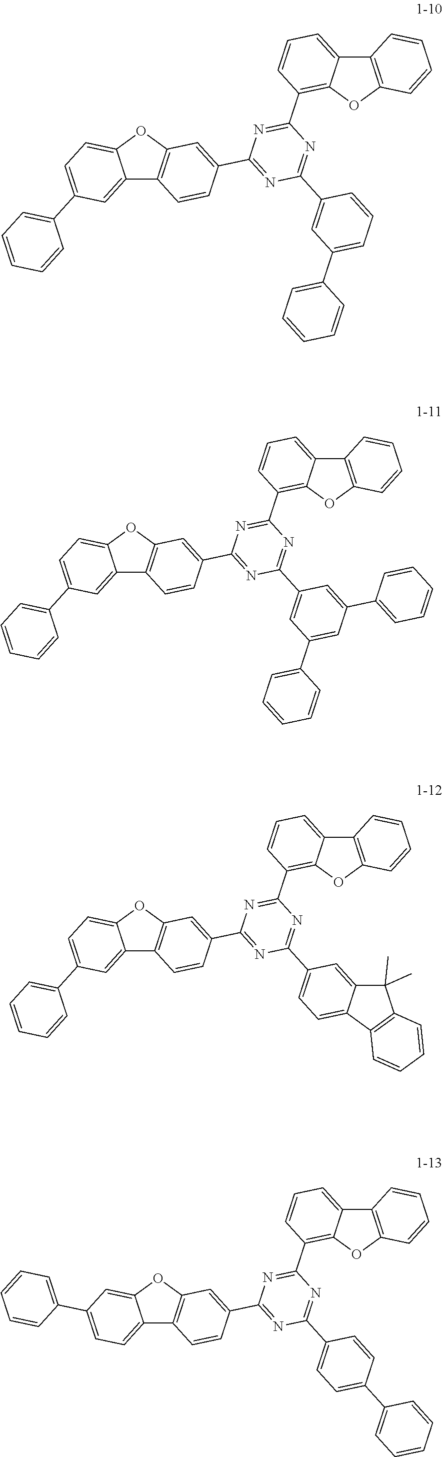 [SMILES: c1ccc(-c2cccc(-c3nc(-c4ccc5c(c4)oc4ccc(-c6ccccc6)cc45)nc(-c4cccc5c4oc4ccccc45)n3)c2)cc1 ; c1ccc(-c2cc(-c3ccccc3)cc(-c3nc(-c4ccc5c(c4)oc4ccc(-c6ccccc6)cc45)nc(-c4cccc5c4oc4ccccc45)n3)c2)cc1 ; CC1(C)c2ccccc2-c2ccc(-c3nc(-c4ccc5c(c4)oc4ccc(-c6ccccc6)cc45)nc(-c4cccc5c4oc4ccccc45)n3)cc21 ; c1ccc(-c2ccc(-c3nc(-c4ccc5c(c4)oc4cc(-c6ccccc6)ccc45)nc(-c4cccc5c4oc4ccccc45)n3)cc2)cc1]

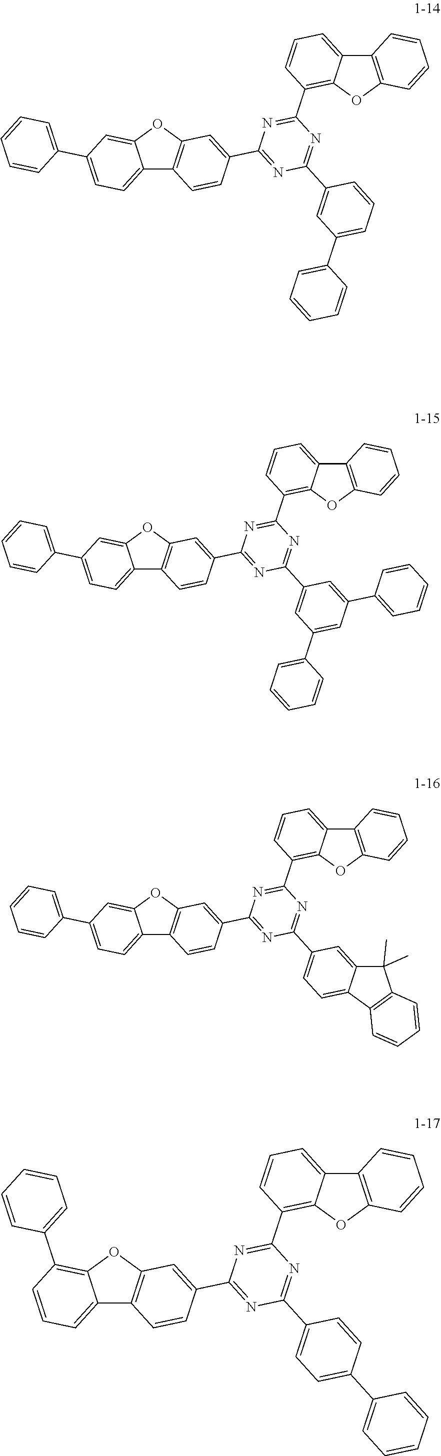 [SMILES: c1ccc(-c2cccc(-c3nc(-c4ccc5c(c4)oc4cc(-c6ccccc6)ccc45)nc(-c4cccc5c4oc4ccccc45)n3)c2)cc1 ; c1ccc(-c2cc(-c3ccccc3)cc(-c3nc(-c4ccc5c(c4)oc4cc(-c6ccccc6)ccc45)nc(-c4cccc5c4oc4ccccc45)n3)c2)cc1 ; CC1(C)c2ccccc2-c2ccc(-c3nc(-c4ccc5c(c4)oc4cc(-c6ccccc6)ccc45)nc(-c4cccc5c4oc4ccccc45)n3)cc21 ; c1ccc(-c2ccc(-c3nc(-c4ccc5c(c4)oc4c(-c6ccccc6)cccc45)nc(-c4cccc5c4oc4ccccc45)n3)cc2)cc1]

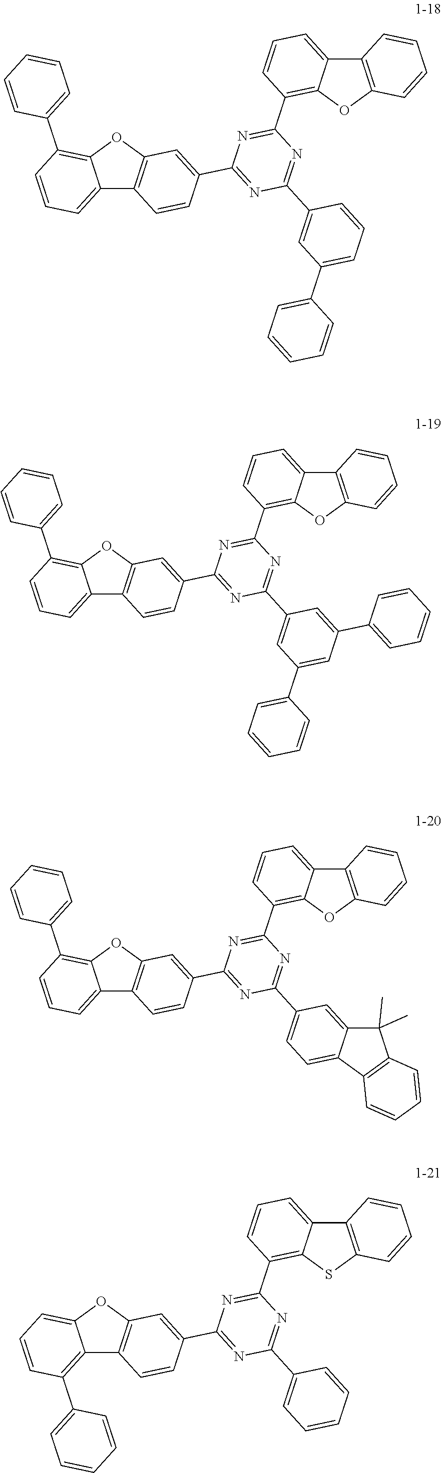 [SMILES: c1ccc(-c2cccc(-c3nc(-c4ccc5c(c4)oc4c(-c6ccccc6)cccc45)nc(-c4cccc5c4oc4ccccc45)n3)c2)cc1 ; c1ccc(-c2cc(-c3ccccc3)cc(-c3nc(-c4ccc5c(c4)oc4c(-c6ccccc6)cccc45)nc(-c4cccc5c4oc4ccccc45)n3)c2)cc1 ; CC1(C)c2ccccc2-c2ccc(-c3nc(-c4ccc5c(c4)oc4c(-c6ccccc6)cccc45)nc(-c4cccc5c4oc4ccccc45)n3)cc21 ; c1ccc(-c2nc(-c3ccc4c(c3)oc3cccc(-c5ccccc5)c34)nc(-c3cccc4c3sc3ccccc34)n2)cc1]

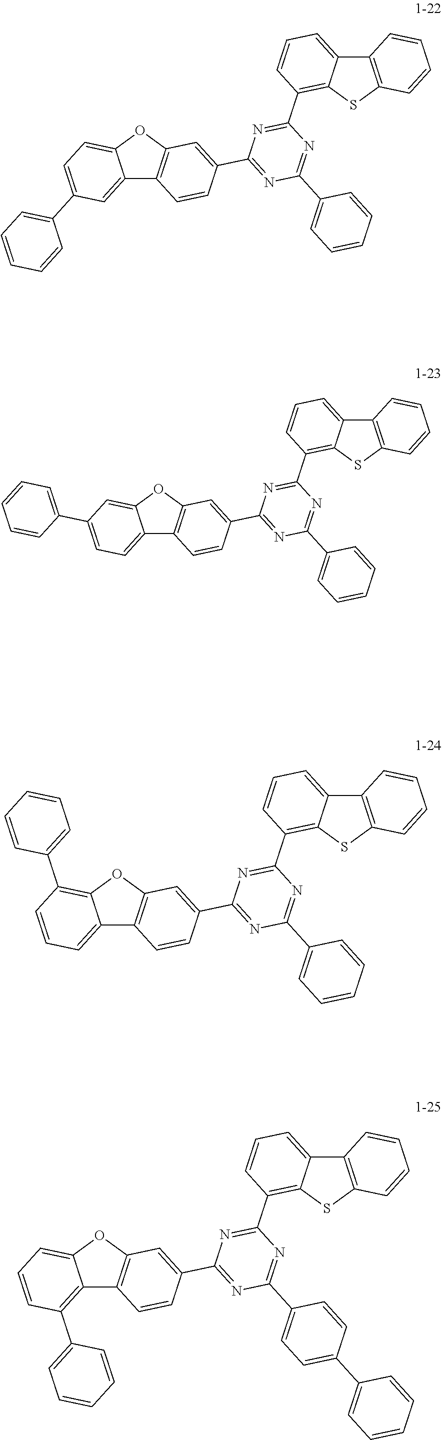 [SMILES: c1ccc(-c2ccc3oc4cc(-c5nc(-c6ccccc6)nc(-c6cccc7c6sc6ccccc67)n5)ccc4c3c2)cc1 ; c1ccc(-c2ccc3c(c2)oc2cc(-c4nc(-c5ccccc5)nc(-c5cccc6c5sc5ccccc56)n4)ccc23)cc1 ; c1ccc(-c2nc(-c3ccc4c(c3)oc3c(-c5ccccc5)cccc34)nc(-c3cccc4c3sc3ccccc34)n2)cc1 ; c1ccc(-c2ccc(-c3nc(-c4ccc5c(c4)oc4cccc(-c6ccccc6)c45)nc(-c4cccc5c4sc4ccccc45)n3)cc2)cc1]

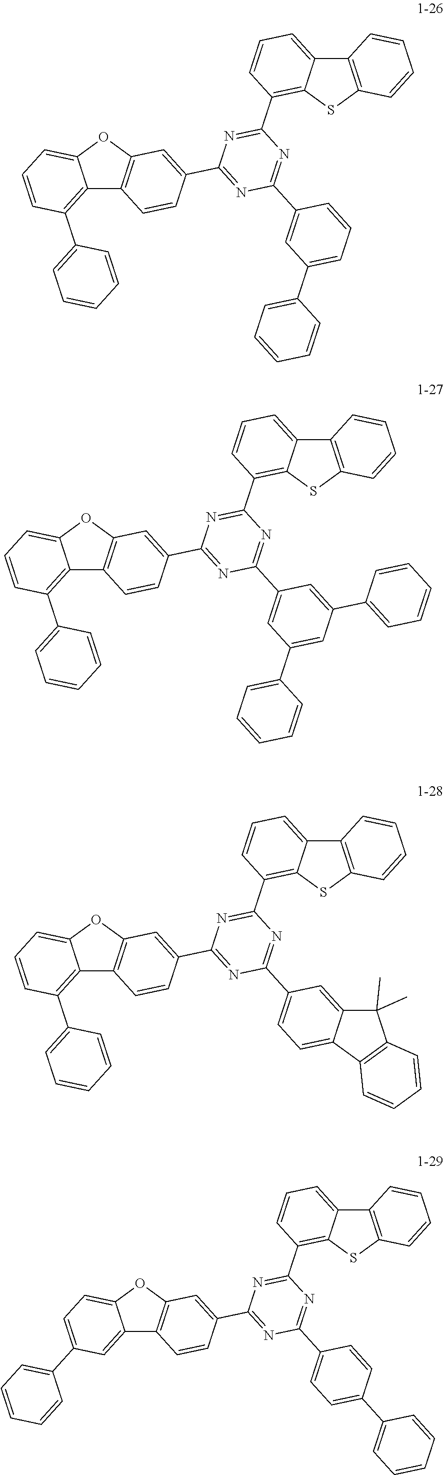 [SMILES: c1ccc(-c2cccc(-c3nc(-c4ccc5c(c4)oc4cccc(-c6ccccc6)c45)nc(-c4cccc5c4sc4ccccc45)n3)c2)cc1 ; c1ccc(-c2cc(-c3ccccc3)cc(-c3nc(-c4ccc5c(c4)oc4cccc(-c6ccccc6)c45)nc(-c4cccc5c4sc4ccccc45)n3)c2)cc1 ; CC1(C)c2ccccc2-c2ccc(-c3nc(-c4ccc5c(c4)oc4cccc(-c6ccccc6)c45)nc(-c4cccc5c4sc4ccccc45)n3)cc21 ; c1ccc(-c2ccc(-c3nc(-c4ccc5c(c4)oc4ccc(-c6ccccc6)cc45)nc(-c4cccc5c4sc4ccccc45)n3)cc2)cc1]

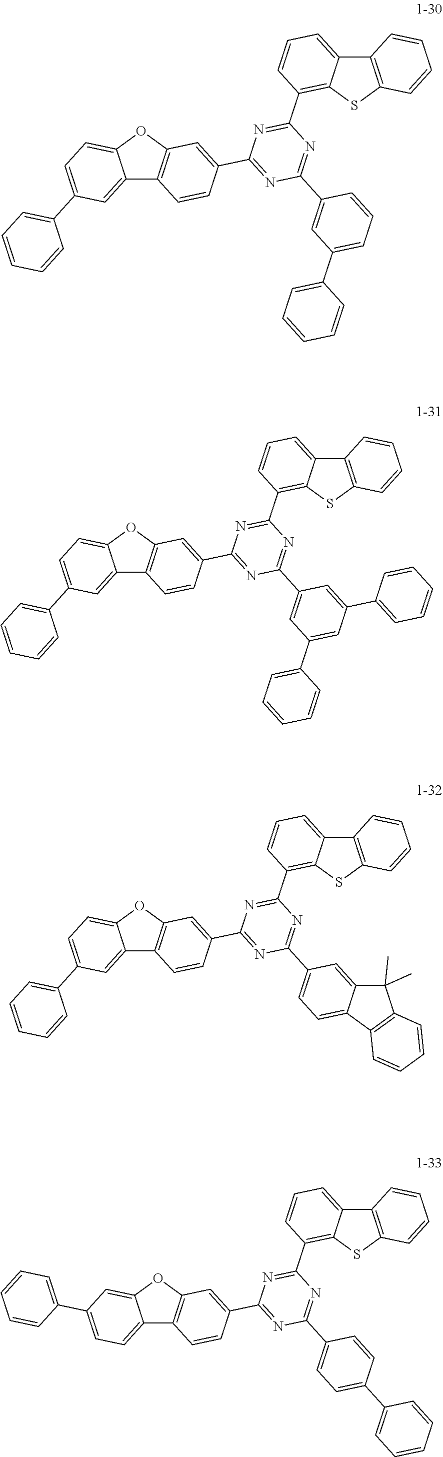 [SMILES: c1ccc(-c2cccc(-c3nc(-c4ccc5c(c4)oc4ccc(-c6ccccc6)cc45)nc(-c4cccc5c4sc4ccccc45)n3)c2)cc1 ; c1ccc(-c2cc(-c3ccccc3)cc(-c3nc(-c4ccc5c(c4)oc4ccc(-c6ccccc6)cc45)nc(-c4cccc5c4sc4ccccc45)n3)c2)cc1 ; CC1(C)c2ccccc2-c2ccc(-c3nc(-c4ccc5c(c4)oc4ccc(-c6ccccc6)cc45)nc(-c4cccc5c4sc4ccccc45)n3)cc21 ; c1ccc(-c2ccc(-c3nc(-c4ccc5c(c4)oc4cc(-c6ccccc6)ccc45)nc(-c4cccc5c4sc4ccccc45)n3)cc2)cc1]

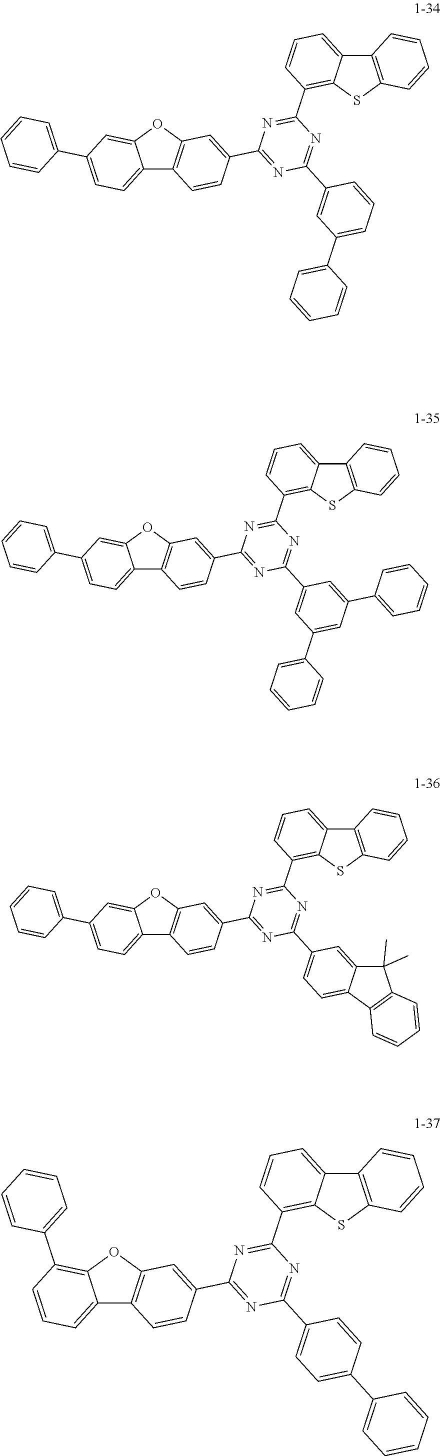 [SMILES: c1ccc(-c2cccc(-c3nc(-c4ccc5c(c4)oc4cc(-c6ccccc6)ccc45)nc(-c4cccc5c4sc4ccccc45)n3)c2)cc1 ; c1ccc(-c2cc(-c3ccccc3)cc(-c3nc(-c4ccc5c(c4)oc4cc(-c6ccccc6)ccc45)nc(-c4cccc5c4sc4ccccc45)n3)c2)cc1 ; CC1(C)c2ccccc2-c2ccc(-c3nc(-c4ccc5c(c4)oc4cc(-c6ccccc6)ccc45)nc(-c4cccc5c4sc4ccccc45)n3)cc21 ; c1ccc(-c2ccc(-c3nc(-c4ccc5c(c4)oc4c(-c6ccccc6)cccc45)nc(-c4cccc5c4sc4ccccc45)n3)cc2)cc1]

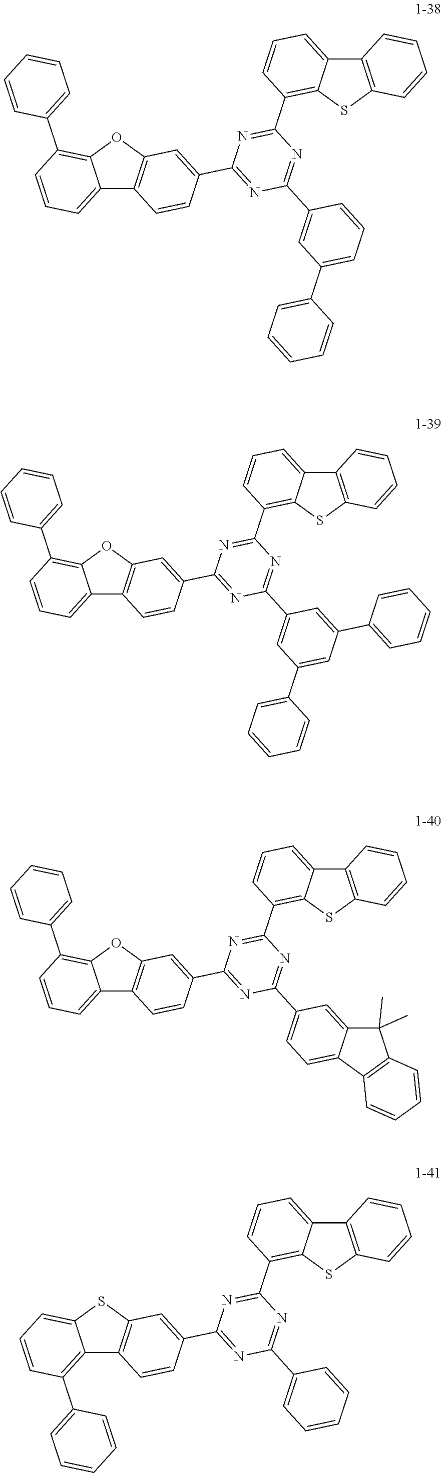 [SMILES: c1ccc(-c2cccc(-c3nc(-c4ccc5c(c4)oc4c(-c6ccccc6)cccc45)nc(-c4cccc5c4sc4ccccc45)n3)c2)cc1 ; c1ccc(-c2cc(-c3ccccc3)cc(-c3nc(-c4ccc5c(c4)oc4c(-c6ccccc6)cccc45)nc(-c4cccc5c4sc4ccccc45)n3)c2)cc1 ; CC1(C)c2ccccc2-c2ccc(-c3nc(-c4ccc5c(c4)oc4c(-c6ccccc6)cccc45)nc(-c4cccc5c4sc4ccccc45)n3)cc21 ; c1ccc(-c2nc(-c3ccc4c(c3)sc3cccc(-c5ccccc5)c34)nc(-c3cccc4c3sc3ccccc34)n2)cc1]

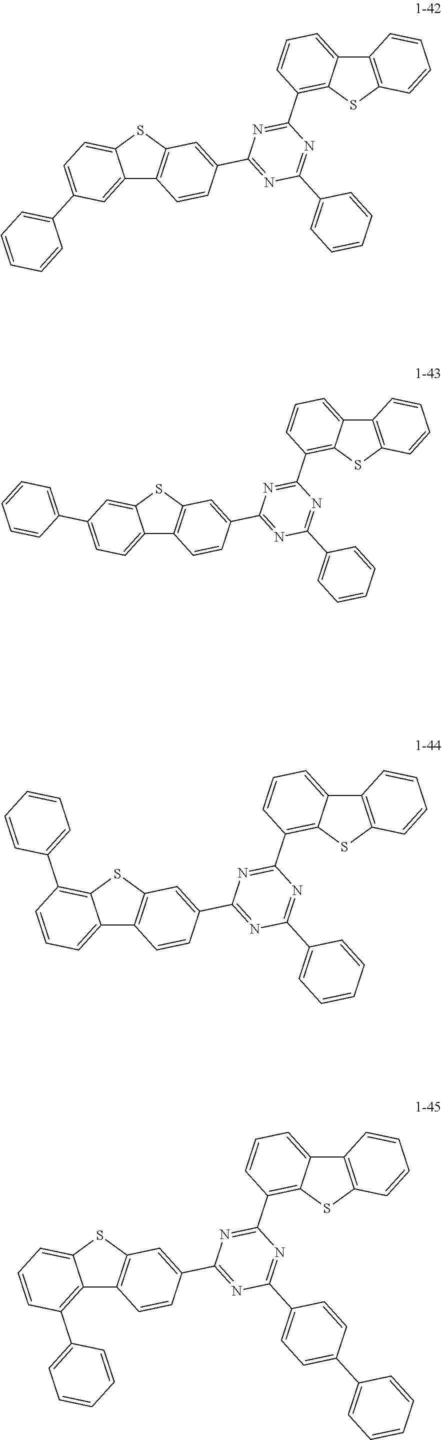 [SMILES: c1ccc(-c2ccc3sc4cc(-c5nc(-c6ccccc6)nc(-c6cccc7c6sc6ccccc67)n5)ccc4c3c2)cc1 ; c1ccc(-c2ccc3c(c2)sc2cc(-c4nc(-c5ccccc5)nc(-c5cccc6c5sc5ccccc56)n4)ccc23)cc1 ; c1ccc(-c2nc(-c3ccc4c(c3)sc3c(-c5ccccc5)cccc34)nc(-c3cccc4c3sc3ccccc34)n2)cc1 ; c1ccc(-c2ccc(-c3nc(-c4ccc5c(c4)sc4cccc(-c6ccccc6)c45)nc(-c4cccc5c4sc4ccccc45)n3)cc2)cc1]

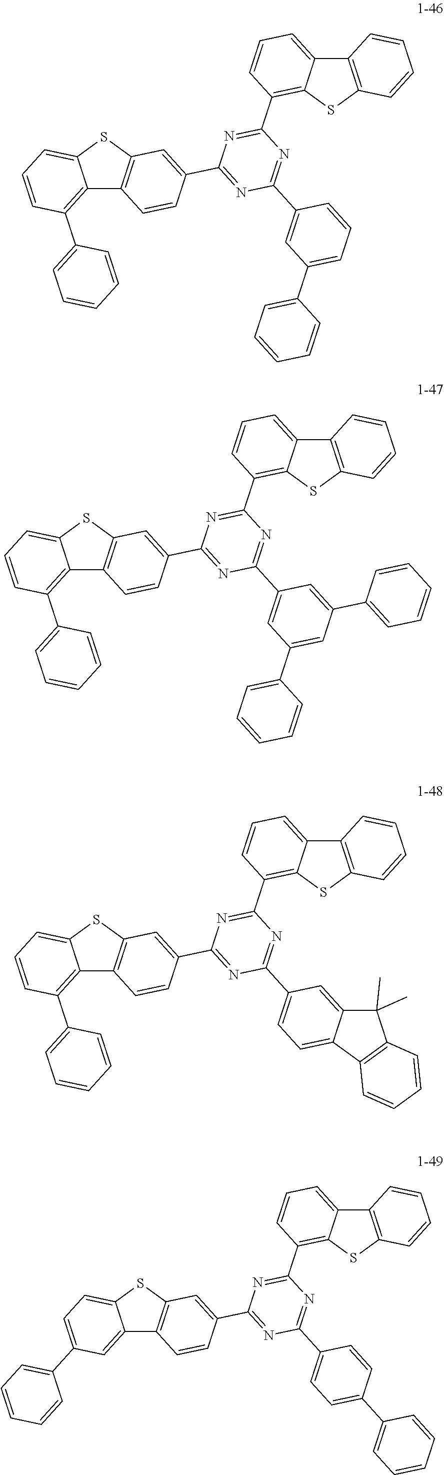 [SMILES: c1ccc(-c2cccc(-c3nc(-c4ccc5c(c4)sc4cccc(-c6ccccc6)c45)nc(-c4cccc5c4sc4ccccc45)n3)c2)cc1 ; c1ccc(-c2cc(-c3ccccc3)cc(-c3nc(-c4ccc5c(c4)sc4cccc(-c6ccccc6)c45)nc(-c4cccc5c4sc4ccccc45)n3)c2)cc1 ; CC1(C)c2ccccc2-c2ccc(-c3nc(-c4ccc5c(c4)sc4cccc(-c6ccccc6)c45)nc(-c4cccc5c4sc4ccccc45)n3)cc21 ; c1ccc(-c2ccc(-c3nc(-c4ccc5c(c4)sc4ccc(-c6ccccc6)cc45)nc(-c4cccc5c4sc4ccccc45)n3)cc2)cc1]

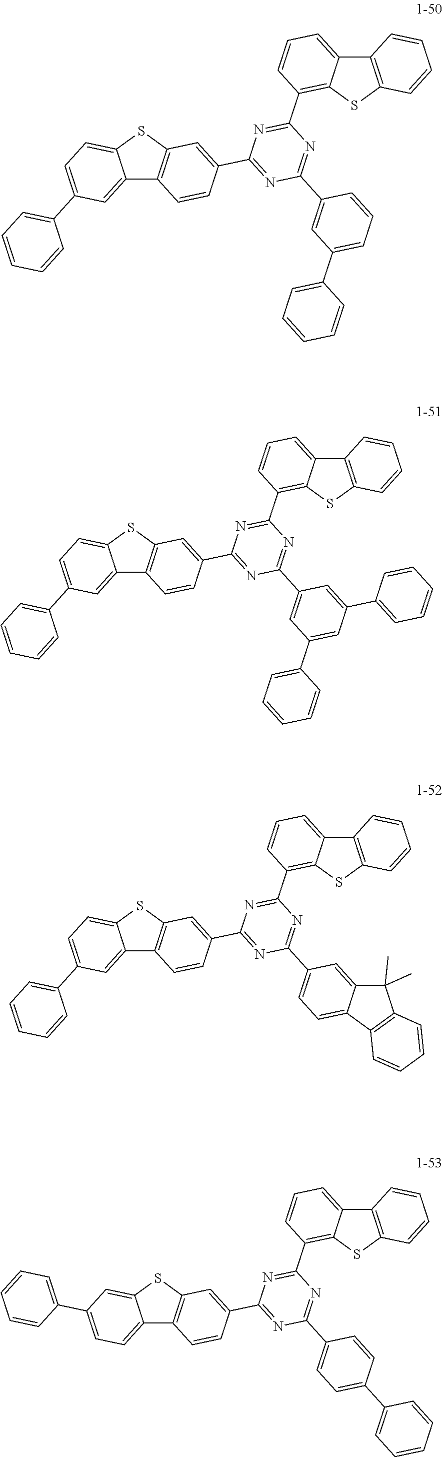 [SMILES: c1ccc(-c2cccc(-c3nc(-c4ccc5c(c4)sc4ccc(-c6ccccc6)cc45)nc(-c4cccc5c4sc4ccccc45)n3)c2)cc1 ; c1ccc(-c2cc(-c3ccccc3)cc(-c3nc(-c4ccc5c(c4)sc4ccc(-c6ccccc6)cc45)nc(-c4cccc5c4sc4ccccc45)n3)c2)cc1 ; CC1(C)c2ccccc2-c2ccc(-c3nc(-c4ccc5c(c4)sc4ccc(-c6ccccc6)cc45)nc(-c4cccc5c4sc4ccccc45)n3)cc21 ; c1ccc(-c2ccc(-c3nc(-c4ccc5c(c4)sc4cc(-c6ccccc6)ccc45)nc(-c4cccc5c4sc4ccccc45)n3)cc2)cc1]

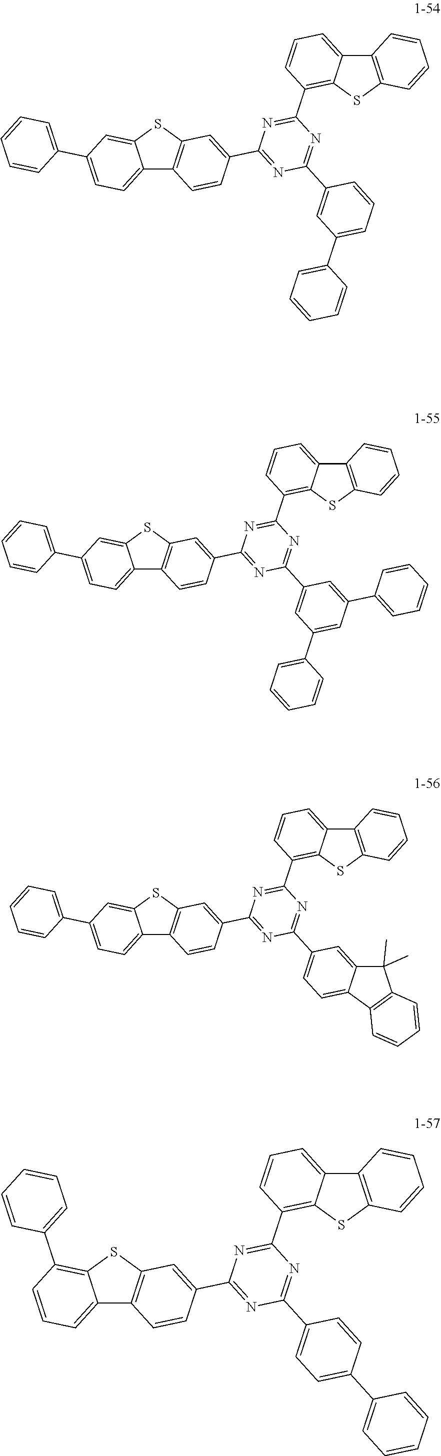 [SMILES: c1ccc(-c2cccc(-c3nc(-c4ccc5c(c4)sc4cc(-c6ccccc6)ccc45)nc(-c4cccc5c4sc4ccccc45)n3)c2)cc1 ; c1ccc(-c2cc(-c3ccccc3)cc(-c3nc(-c4ccc5c(c4)sc4cc(-c6ccccc6)ccc45)nc(-c4cccc5c4sc4ccccc45)n3)c2)cc1 ; CC1(C)c2ccccc2-c2ccc(-c3nc(-c4ccc5c(c4)sc4cc(-c6ccccc6)ccc45)nc(-c4cccc5c4sc4ccccc45)n3)cc21 ; c1ccc(-c2ccc(-c3nc(-c4ccc5c(c4)sc4c(-c6ccccc6)cccc45)nc(-c4cccc5c4sc4ccccc45)n3)cc2)cc1]

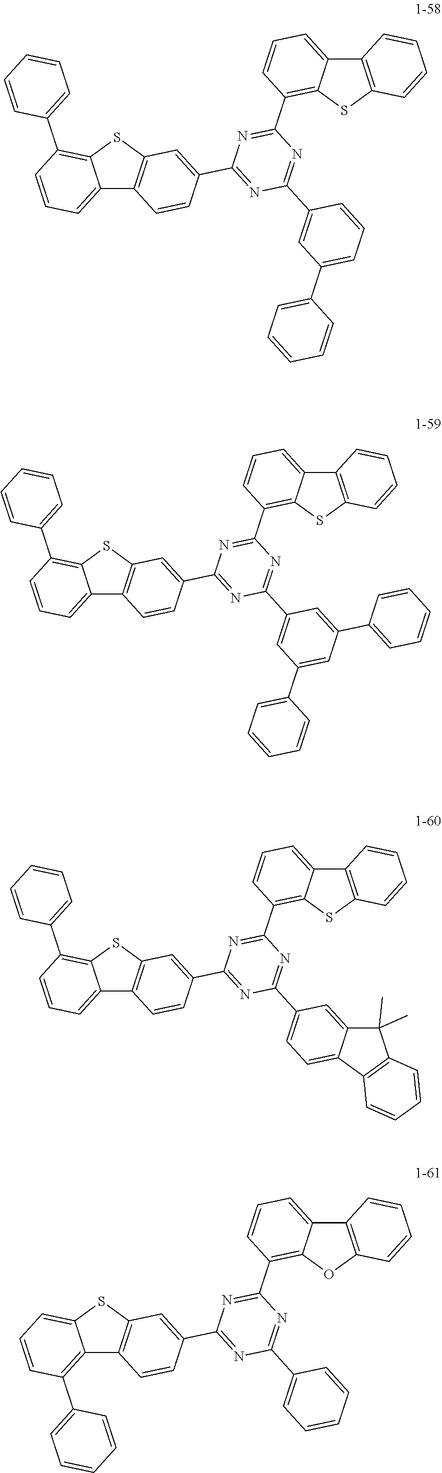 [SMILES: c1ccc(-c2cccc(-c3nc(-c4ccc5c(c4)sc4c(-c6ccccc6)cccc45)nc(-c4cccc5c4sc4ccccc45)n3)c2)cc1 ; c1ccc(-c2cc(-c3ccccc3)cc(-c3nc(-c4ccc5c(c4)sc4c(-c6ccccc6)cccc45)nc(-c4cccc5c4sc4ccccc45)n3)c2)cc1 ; CC1(C)c2ccccc2-c2ccc(-c3nc(-c4ccc5c(c4)sc4c(-c6ccccc6)cccc45)nc(-c4cccc5c4sc4ccccc45)n3)cc21 ; c1ccc(-c2nc(-c3ccc4c(c3)sc3cccc(-c5ccccc5)c34)nc(-c3cccc4c3oc3ccccc34)n2)cc1]

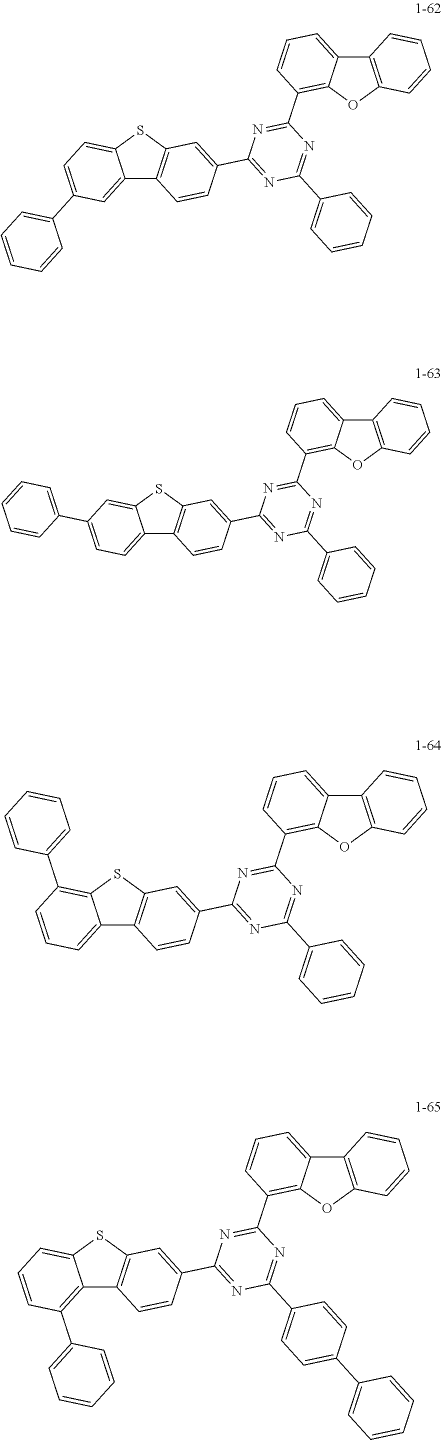 [SMILES: c1ccc(-c2ccc3sc4cc(-c5nc(-c6ccccc6)nc(-c6cccc7c6oc6ccccc67)n5)ccc4c3c2)cc1 ; c1ccc(-c2ccc3c(c2)sc2cc(-c4nc(-c5ccccc5)nc(-c5cccc6c5oc5ccccc56)n4)ccc23)cc1 ; c1ccc(-c2nc(-c3ccc4c(c3)sc3c(-c5ccccc5)cccc34)nc(-c3cccc4c3oc3ccccc34)n2)cc1 ; c1ccc(-c2ccc(-c3nc(-c4ccc5c(c4)sc4cccc(-c6ccccc6)c45)nc(-c4cccc5c4oc4ccccc45)n3)cc2)cc1]

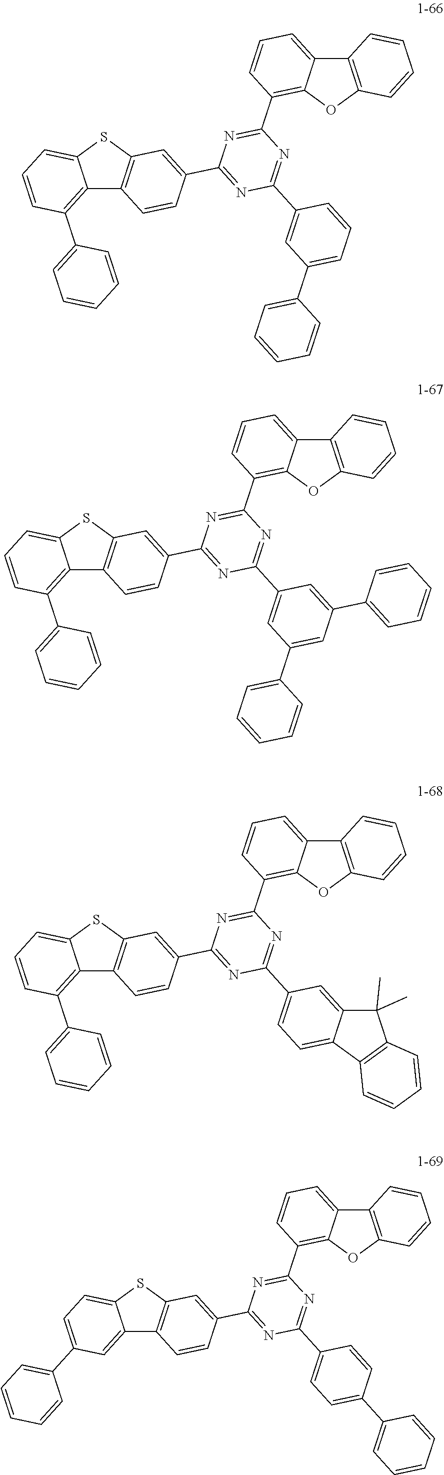 [SMILES: c1ccc(-c2cccc(-c3nc(-c4ccc5c(c4)sc4cccc(-c6ccccc6)c45)nc(-c4cccc5c4oc4ccccc45)n3)c2)cc1 ; c1ccc(-c2cc(-c3ccccc3)cc(-c3nc(-c4ccc5c(c4)sc4cccc(-c6ccccc6)c45)nc(-c4cccc5c4oc4ccccc45)n3)c2)cc1 ; CC1(C)c2ccccc2-c2ccc(-c3nc(-c4ccc5c(c4)sc4cccc(-c6ccccc6)c45)nc(-c4cccc5c4oc4ccccc45)n3)cc21 ; c1ccc(-c2ccc(-c3nc(-c4ccc5c(c4)sc4ccc(-c6ccccc6)cc45)nc(-c4cccc5c4oc4ccccc45)n3)cc2)cc1]

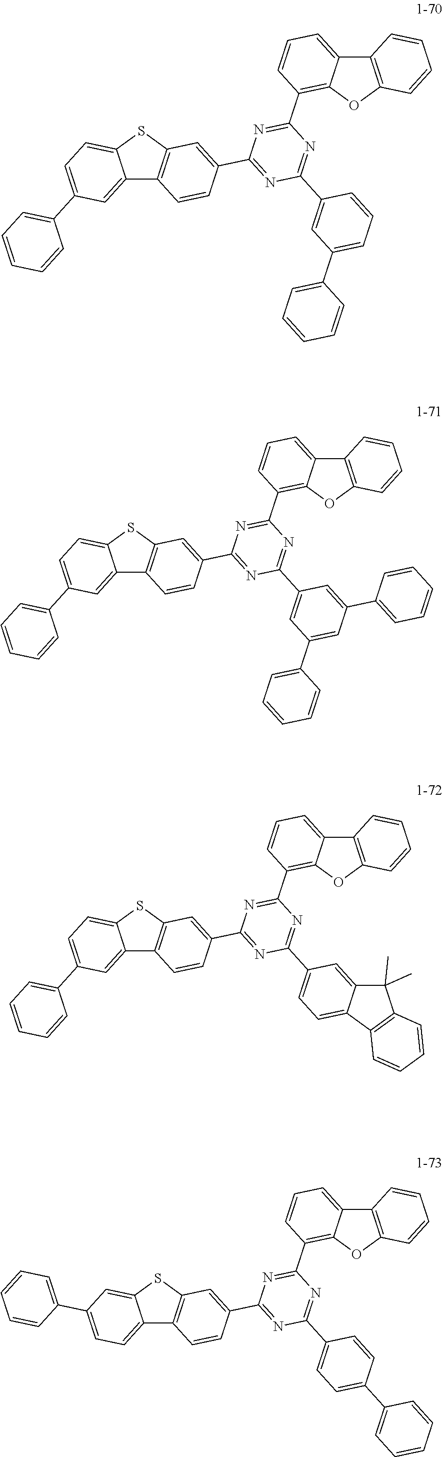 [SMILES: c1ccc(-c2cccc(-c3nc(-c4ccc5c(c4)sc4ccc(-c6ccccc6)cc45)nc(-c4cccc5c4oc4ccccc45)n3)c2)cc1 ; c1ccc(-c2cc(-c3ccccc3)cc(-c3nc(-c4ccc5c(c4)sc4ccc(-c6ccccc6)cc45)nc(-c4cccc5c4oc4ccccc45)n3)c2)cc1 ; CC1(C)c2ccccc2-c2ccc(-c3nc(-c4ccc5c(c4)sc4ccc(-c6ccccc6)cc45)nc(-c4cccc5c4oc4ccccc45)n3)cc21 ; c1ccc(-c2ccc(-c3nc(-c4ccc5c(c4)sc4cc(-c6ccccc6)ccc45)nc(-c4cccc5c4oc4ccccc45)n3)cc2)cc1]

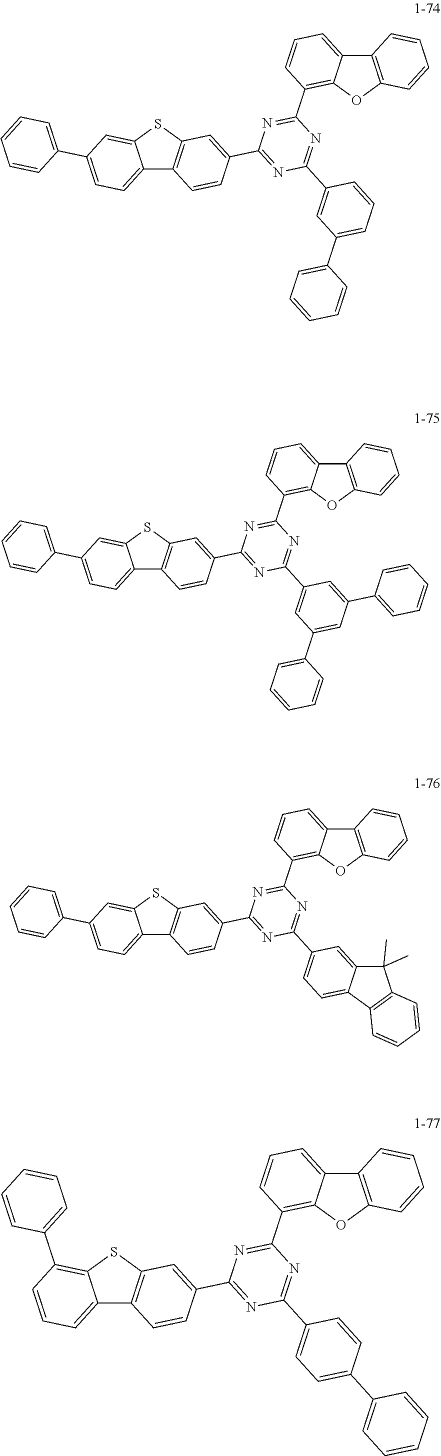 [SMILES: c1ccc(-c2cccc(-c3nc(-c4ccc5c(c4)sc4cc(-c6ccccc6)ccc45)nc(-c4cccc5c4oc4ccccc45)n3)c2)cc1 ; c1ccc(-c2cc(-c3ccccc3)cc(-c3nc(-c4ccc5c(c4)sc4cc(-c6ccccc6)ccc45)nc(-c4cccc5c4oc4ccccc45)n3)c2)cc1 ; CC1(C)c2ccccc2-c2ccc(-c3nc(-c4ccc5c(c4)sc4cc(-c6ccccc6)ccc45)nc(-c4cccc5c4oc4ccccc45)n3)cc21 ; c1ccc(-c2ccc(-c3nc(-c4ccc5c(c4)sc4c(-c6ccccc6)cccc45)nc(-c4cccc5c4oc4ccccc45)n3)cc2)cc1]

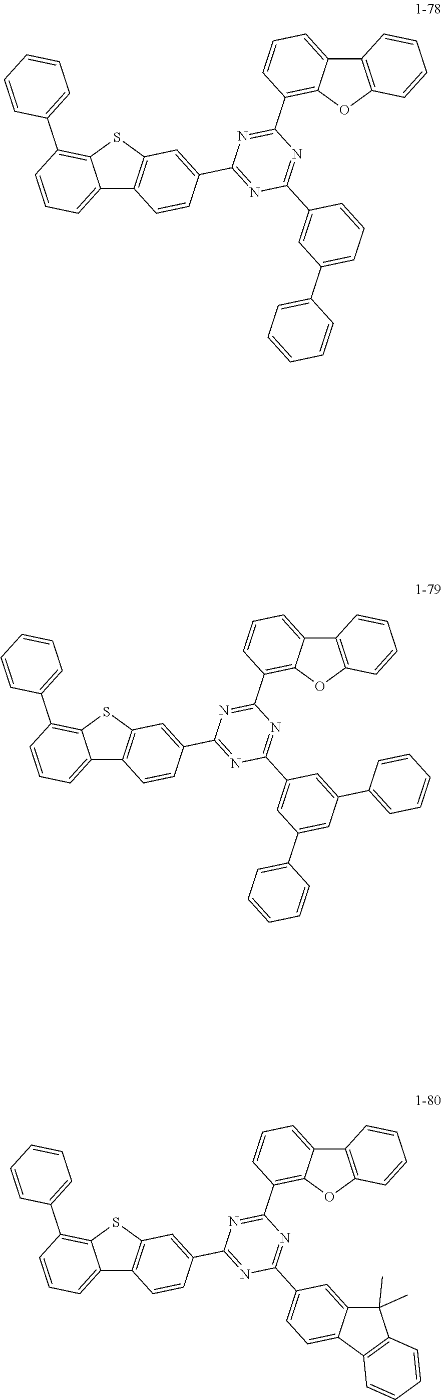 [SMILES: c1ccc(-c2cccc(-c3nc(-c4ccc5c(c4)sc4c(-c6ccccc6)cccc45)nc(-c4cccc5c4oc4ccccc45)n3)c2)cc1 ; c1ccc(-c2cc(-c3ccccc3)cc(-c3nc(-c4ccc5c(c4)sc4c(-c6ccccc6)cccc45)nc(-c4cccc5c4oc4ccccc45)n3)c2)cc1 ; CC1(C)c2ccccc2-c2ccc(-c3nc(-c4ccc5c(c4)sc4c(-c6ccccc6)cccc45)nc(-c4cccc5c4oc4ccccc45)n3)cc21]

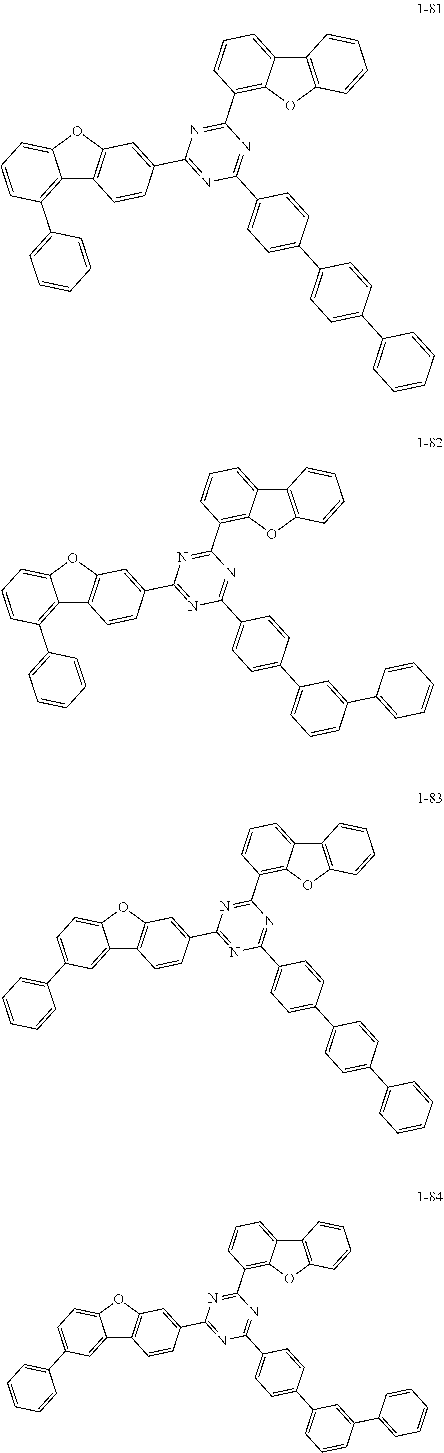 [SMILES: c1ccc(-c2ccc(-c3ccc(-c4nc(-c5ccc6c(c5)oc5cccc(-c7ccccc7)c56)nc(-c5cccc6c5oc5ccccc56)n4)cc3)cc2)cc1 ; c1ccc(-c2cccc(-c3ccc(-c4nc(-c5ccc6c(c5)oc5cccc(-c7ccccc7)c56)nc(-c5cccc6c5oc5ccccc56)n4)cc3)c2)cc1 ; c1ccc(-c2ccc(-c3ccc(-c4nc(-c5ccc6c(c5)oc5ccc(-c7ccccc7)cc56)nc(-c5cccc6c5oc5ccccc56)n4)cc3)cc2)cc1 ; c1ccc(-c2cccc(-c3ccc(-c4nc(-c5ccc6c(c5)oc5ccc(-c7ccccc7)cc56)nc(-c5cccc6c5oc5ccccc56)n4)cc3)c2)cc1]

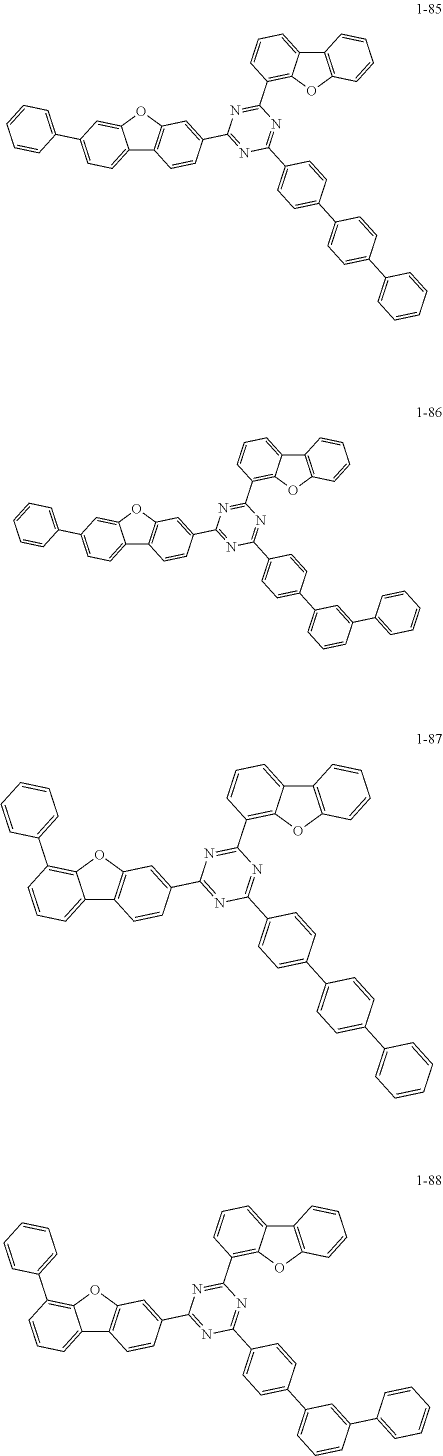 [SMILES: c1ccc(-c2ccc(-c3ccc(-c4nc(-c5ccc6c(c5)oc5cc(-c7ccccc7)ccc56)nc(-c5cccc6c5oc5ccccc56)n4)cc3)cc2)cc1 ; c1ccc(-c2cccc(-c3ccc(-c4nc(-c5ccc6c(c5)oc5cc(-c7ccccc7)ccc56)nc(-c5cccc6c5oc5ccccc56)n4)cc3)c2)cc1 ; c1ccc(-c2ccc(-c3ccc(-c4nc(-c5ccc6c(c5)oc5c(-c7ccccc7)cccc56)nc(-c5cccc6c5oc5ccccc56)n4)cc3)cc2)cc1 ; c1ccc(-c2cccc(-c3ccc(-c4nc(-c5ccc6c(c5)oc5c(-c7ccccc7)cccc56)nc(-c5cccc6c5oc5ccccc56)n4)cc3)c2)cc1]

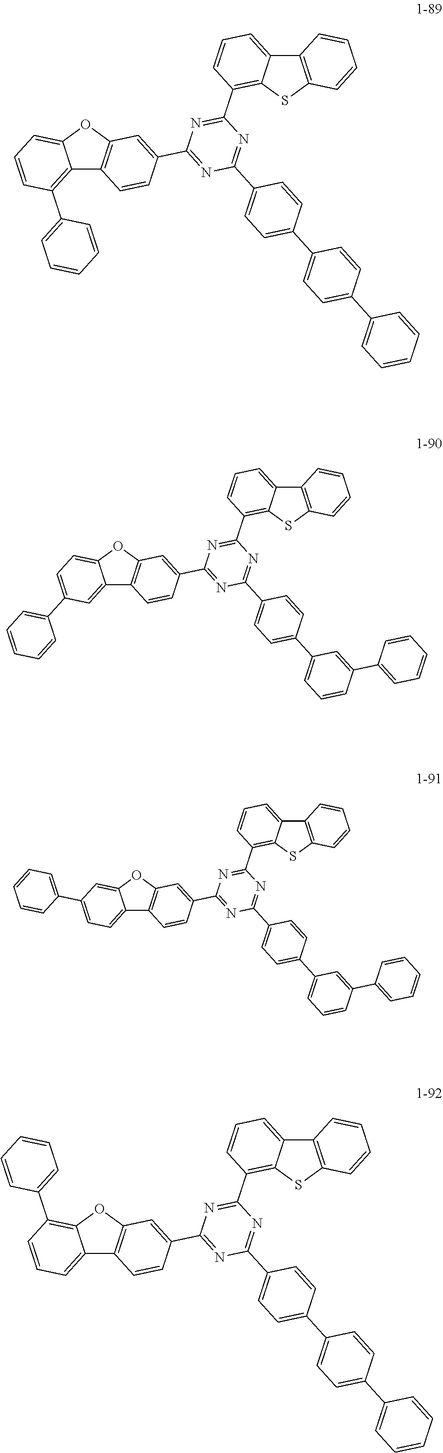 [SMILES: c1ccc(-c2ccc(-c3ccc(-c4nc(-c5ccc6c(c5)oc5cccc(-c7ccccc7)c56)nc(-c5cccc6c5sc5ccccc56)n4)cc3)cc2)cc1 ; c1ccc(-c2cccc(-c3ccc(-c4nc(-c5ccc6c(c5)oc5ccc(-c7ccccc7)cc56)nc(-c5cccc6c5sc5ccccc56)n4)cc3)c2)cc1 ; c1ccc(-c2cccc(-c3ccc(-c4nc(-c5ccc6c(c5)oc5cc(-c7ccccc7)ccc56)nc(-c5cccc6c5sc5ccccc56)n4)cc3)c2)cc1 ; c1ccc(-c2ccc(-c3ccc(-c4nc(-c5ccc6c(c5)oc5c(-c7ccccc7)cccc56)nc(-c5cccc6c5sc5ccccc56)n4)cc3)cc2)cc1]

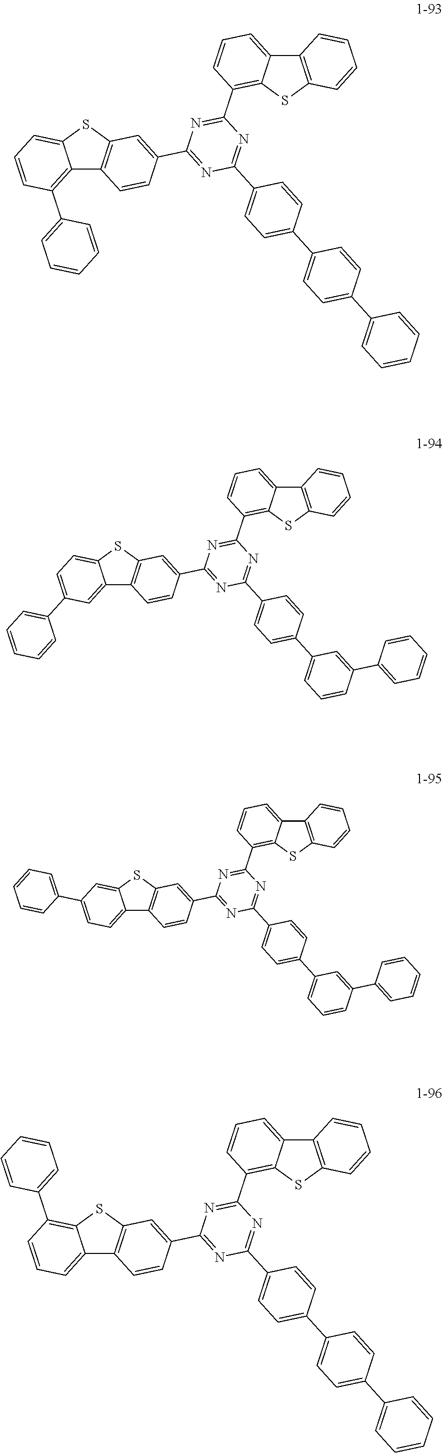 [SMILES: c1ccc(-c2ccc(-c3ccc(-c4nc(-c5ccc6c(c5)sc5cccc(-c7ccccc7)c56)nc(-c5cccc6c5sc5ccccc56)n4)cc3)cc2)cc1 ; c1ccc(-c2cccc(-c3ccc(-c4nc(-c5ccc6c(c5)sc5ccc(-c7ccccc7)cc56)nc(-c5cccc6c5sc5ccccc56)n4)cc3)c2)cc1 ; c1ccc(-c2cccc(-c3ccc(-c4nc(-c5ccc6c(c5)sc5cc(-c7ccccc7)ccc56)nc(-c5cccc6c5sc5ccccc56)n4)cc3)c2)cc1 ; c1ccc(-c2ccc(-c3ccc(-c4nc(-c5ccc6c(c5)sc5c(-c7ccccc7)cccc56)nc(-c5cccc6c5sc5ccccc56)n4)cc3)cc2)cc1]

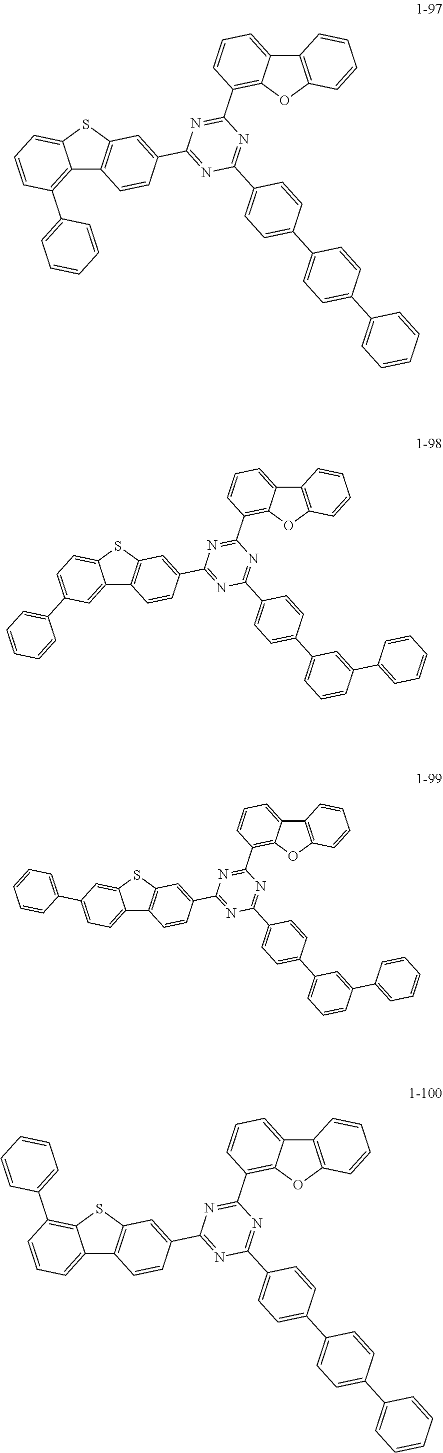 [SMILES: c1ccc(-c2ccc(-c3ccc(-c4nc(-c5ccc6c(c5)sc5cccc(-c7ccccc7)c56)nc(-c5cccc6c5oc5ccccc56)n4)cc3)cc2)cc1 ; c1ccc(-c2cccc(-c3ccc(-c4nc(-c5ccc6c(c5)sc5ccc(-c7ccccc7)cc56)nc(-c5cccc6c5oc5ccccc56)n4)cc3)c2)cc1 ; c1ccc(-c2cccc(-c3ccc(-c4nc(-c5ccc6c(c5)sc5cc(-c7ccccc7)ccc56)nc(-c5cccc6c5oc5ccccc56)n4)cc3)c2)cc1 ; c1ccc(-c2ccc(-c3ccc(-c4nc(-c5ccc6c(c5)sc5c(-c7ccccc7)cccc56)nc(-c5cccc6c5oc5ccccc56)n4)cc3)cc2)cc1]

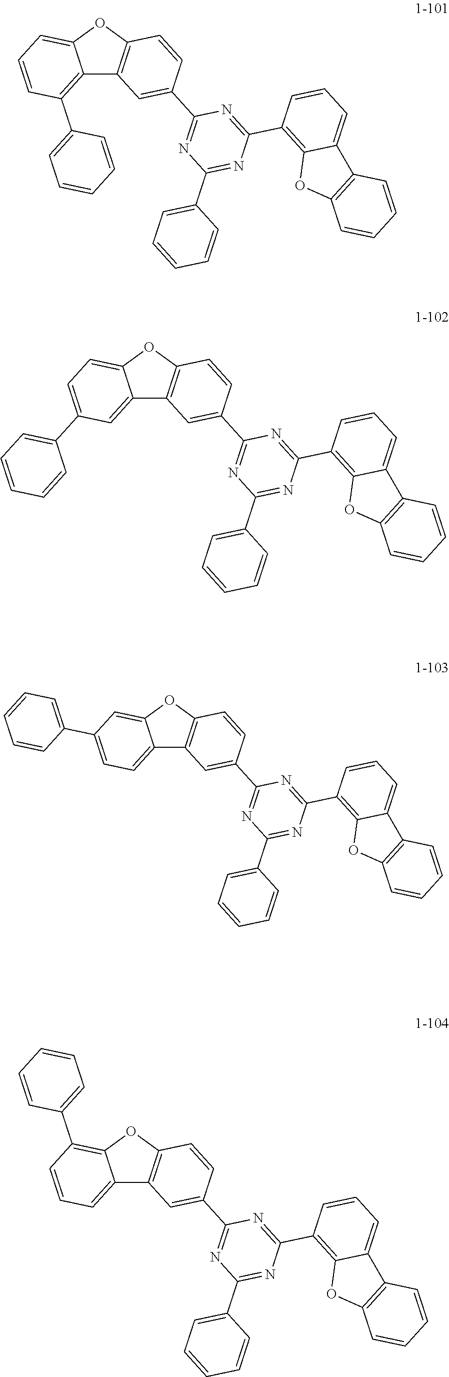 [SMILES: c1ccc(-c2nc(-c3ccc4oc5cccc(-c6ccccc6)c5c4c3)nc(-c3cccc4c3oc3ccccc34)n2)cc1 ; c1ccc(-c2ccc3oc4ccc(-c5nc(-c6ccccc6)nc(-c6cccc7c6oc6ccccc67)n5)cc4c3c2)cc1 ; c1ccc(-c2ccc3c(c2)oc2ccc(-c4nc(-c5ccccc5)nc(-c5cccc6c5oc5ccccc56)n4)cc23)cc1 ; c1ccc(-c2nc(-c3ccc4oc5c(-c6ccccc6)cccc5c4c3)nc(-c3cccc4c3oc3ccccc34)n2)cc1]

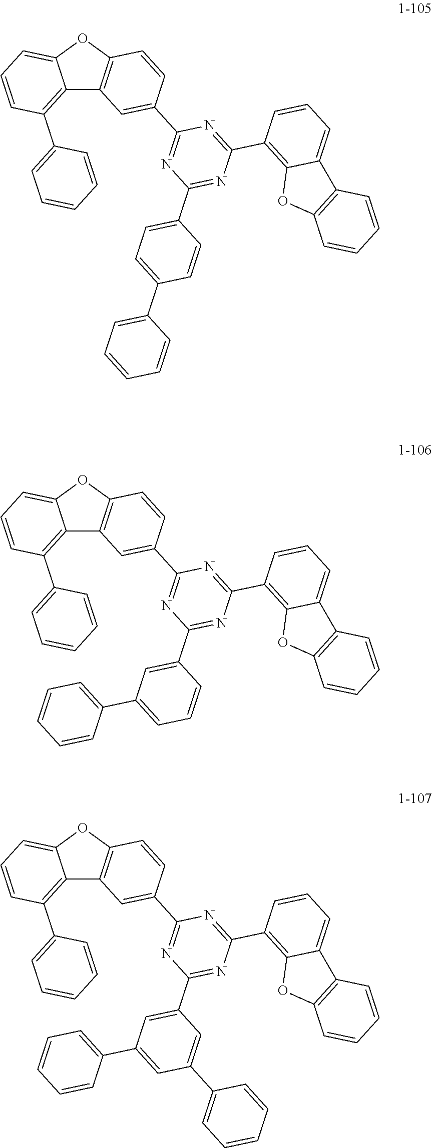 [SMILES: c1ccc(-c2ccc(-c3nc(-c4ccc5oc6cccc(-c7ccccc7)c6c5c4)nc(-c4cccc5c4oc4ccccc45)n3)cc2)cc1 ; c1ccc(-c2cccc(-c3nc(-c4ccc5oc6cccc(-c7ccccc7)c6c5c4)nc(-c4cccc5c4oc4ccccc45)n3)c2)cc1 ; c1ccc(-c2cc(-c3ccccc3)cc(-c3nc(-c4ccc5oc6cccc(-c7ccccc7)c6c5c4)nc(-c4cccc5c4oc4ccccc45)n3)c2)cc1]

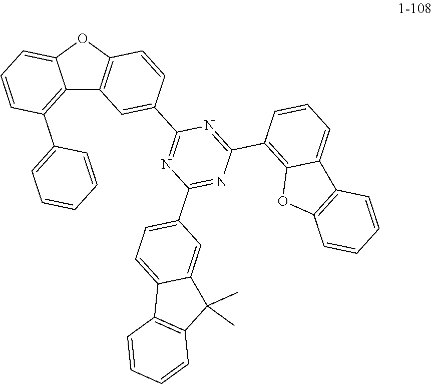 [SMILES: CC1(C)c2ccccc2-c2ccc(-c3nc(-c4ccc5oc6cccc(-c7ccccc7)c6c5c4)nc(-c4cccc5c4oc4ccccc45)n3)cc21]

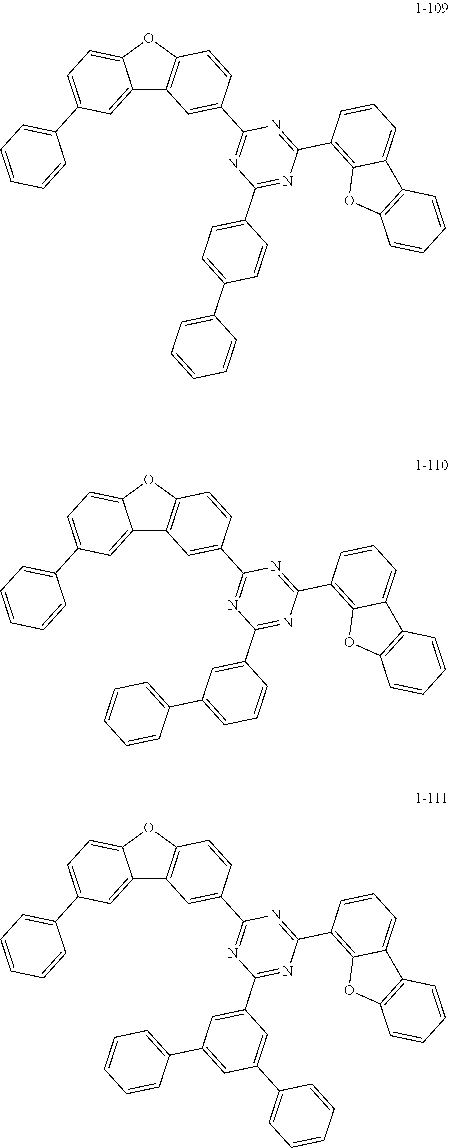 [SMILES: c1ccc(-c2ccc(-c3nc(-c4ccc5oc6ccc(-c7ccccc7)cc6c5c4)nc(-c4cccc5c4oc4ccccc45)n3)cc2)cc1 ; c1ccc(-c2cccc(-c3nc(-c4ccc5oc6ccc(-c7ccccc7)cc6c5c4)nc(-c4cccc5c4oc4ccccc45)n3)c2)cc1 ; c1ccc(-c2cc(-c3ccccc3)cc(-c3nc(-c4ccc5oc6ccc(-c7ccccc7)cc6c5c4)nc(-c4cccc5c4oc4ccccc45)n3)c2)cc1]

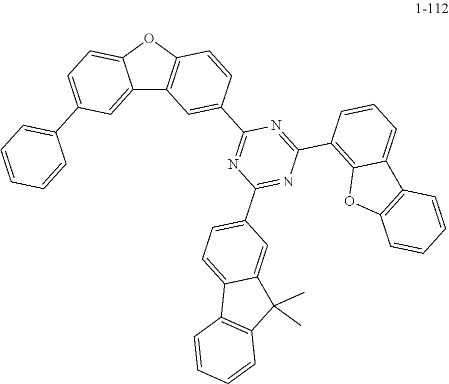 [SMILES: CC1(C)c2ccccc2-c2ccc(-c3nc(-c4ccc5oc6ccc(-c7ccccc7)cc6c5c4)nc(-c4cccc5c4oc4ccccc45)n3)cc21]

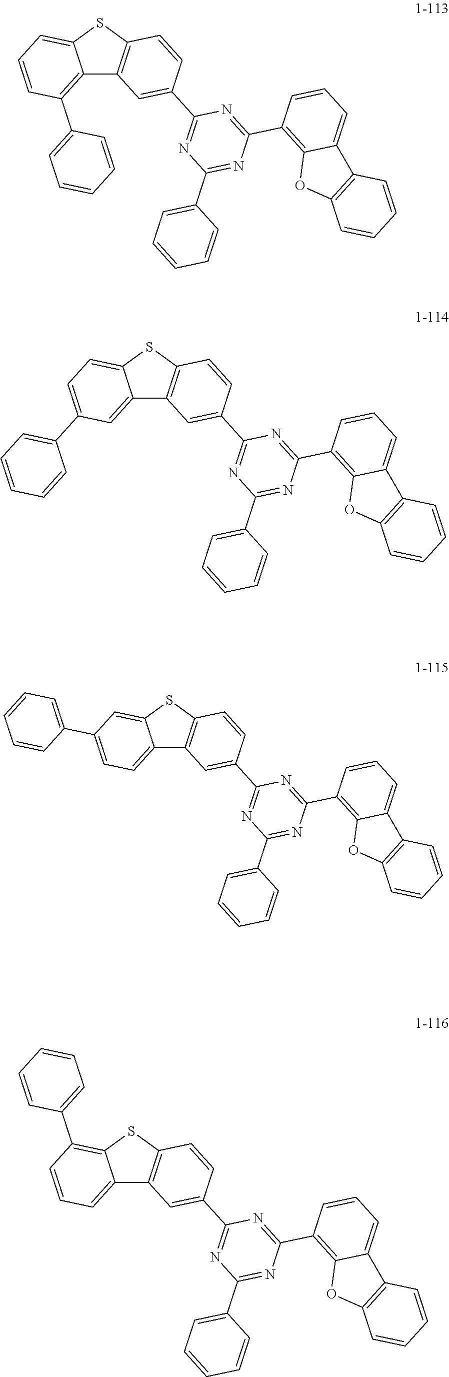 [SMILES: c1ccc(-c2nc(-c3ccc4sc5cccc(-c6ccccc6)c5c4c3)nc(-c3cccc4c3oc3ccccc34)n2)cc1 ; c1ccc(-c2ccc3sc4ccc(-c5nc(-c6ccccc6)nc(-c6cccc7c6oc6ccccc67)n5)cc4c3c2)cc1 ; c1ccc(-c2ccc3c(c2)sc2ccc(-c4nc(-c5ccccc5)nc(-c5cccc6c5oc5ccccc56)n4)cc23)cc1 ; c1ccc(-c2nc(-c3ccc4sc5c(-c6ccccc6)cccc5c4c3)nc(-c3cccc4c3oc3ccccc34)n2)cc1]

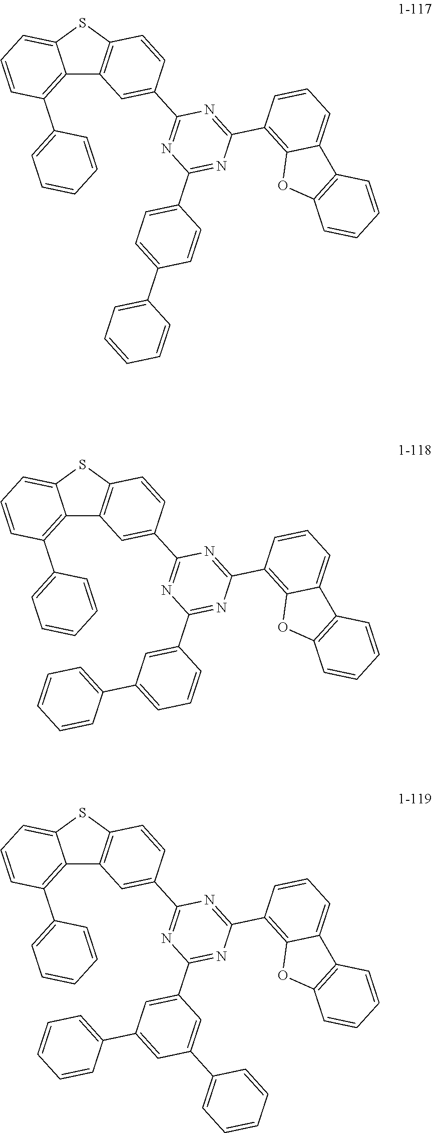 [SMILES: c1ccc(-c2ccc(-c3nc(-c4ccc5sc6cccc(-c7ccccc7)c6c5c4)nc(-c4cccc5c4oc4ccccc45)n3)cc2)cc1 ; c1ccc(-c2cccc(-c3nc(-c4ccc5sc6cccc(-c7ccccc7)c6c5c4)nc(-c4cccc5c4oc4ccccc45)n3)c2)cc1 ; c1ccc(-c2cc(-c3ccccc3)cc(-c3nc(-c4ccc5sc6cccc(-c7ccccc7)c6c5c4)nc(-c4cccc5c4oc4ccccc45)n3)c2)cc1]

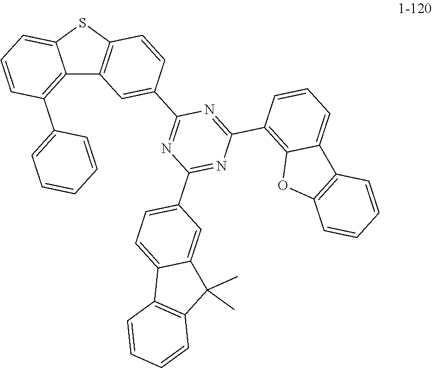 [SMILES: CC1(C)c2ccccc2-c2ccc(-c3nc(-c4ccc5sc6cccc(-c7ccccc7)c6c5c4)nc(-c4cccc5c4oc4ccccc45)n3)cc21]

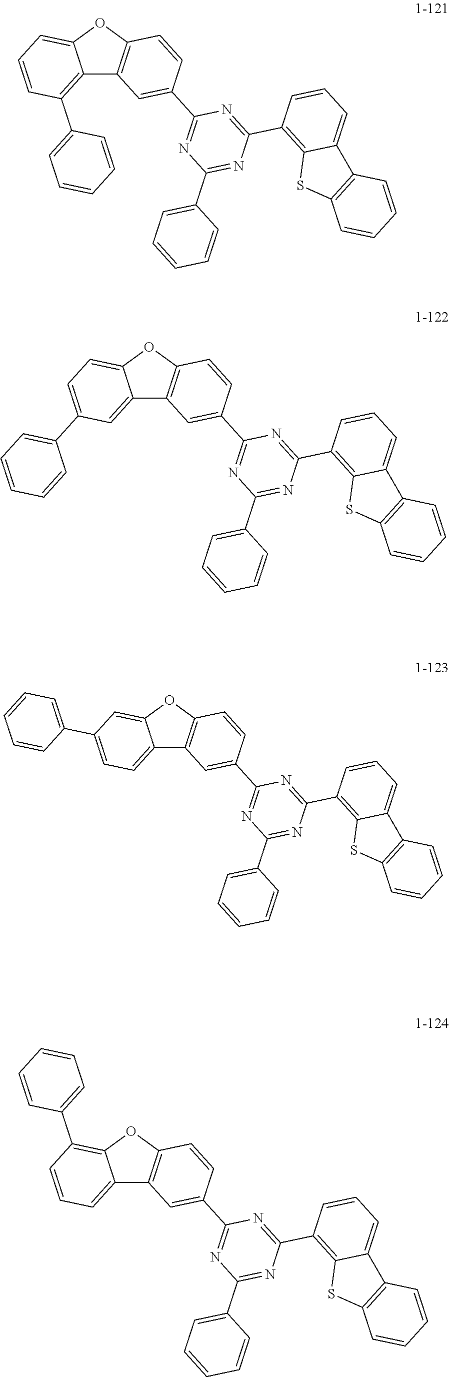 [SMILES: c1ccc(-c2nc(-c3ccc4oc5cccc(-c6ccccc6)c5c4c3)nc(-c3cccc4c3sc3ccccc34)n2)cc1 ; c1ccc(-c2ccc3oc4ccc(-c5nc(-c6ccccc6)nc(-c6cccc7c6sc6ccccc67)n5)cc4c3c2)cc1 ; c1ccc(-c2ccc3c(c2)oc2ccc(-c4nc(-c5ccccc5)nc(-c5cccc6c5sc5ccccc56)n4)cc23)cc1 ; c1ccc(-c2nc(-c3ccc4oc5c(-c6ccccc6)cccc5c4c3)nc(-c3cccc4c3sc3ccccc34)n2)cc1]

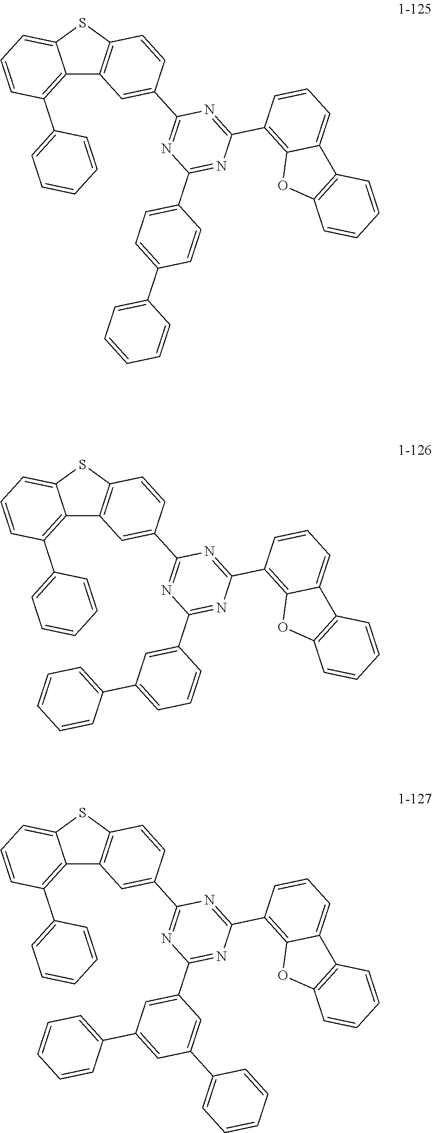 [SMILES: c1ccc(-c2ccc(-c3nc(-c4ccc5sc6cccc(-c7ccccc7)c6c5c4)nc(-c4cccc5c4oc4ccccc45)n3)cc2)cc1 ; c1ccc(-c2cccc(-c3nc(-c4ccc5sc6cccc(-c7ccccc7)c6c5c4)nc(-c4cccc5c4oc4ccccc45)n3)c2)cc1 ; c1ccc(-c2cc(-c3ccccc3)cc(-c3nc(-c4ccc5sc6cccc(-c7ccccc7)c6c5c4)nc(-c4cccc5c4oc4ccccc45)n3)c2)cc1]

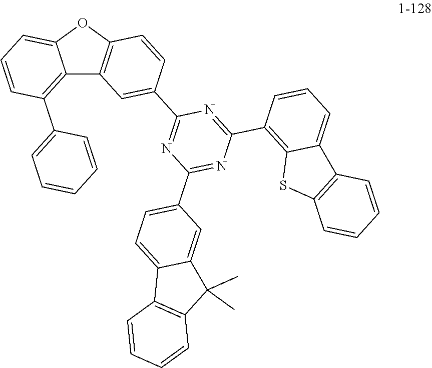 [SMILES: CC1(C)c2ccccc2-c2ccc(-c3nc(-c4ccc5oc6cccc(-c7ccccc7)c6c5c4)nc(-c4cccc5c4sc4ccccc45)n3)cc21]

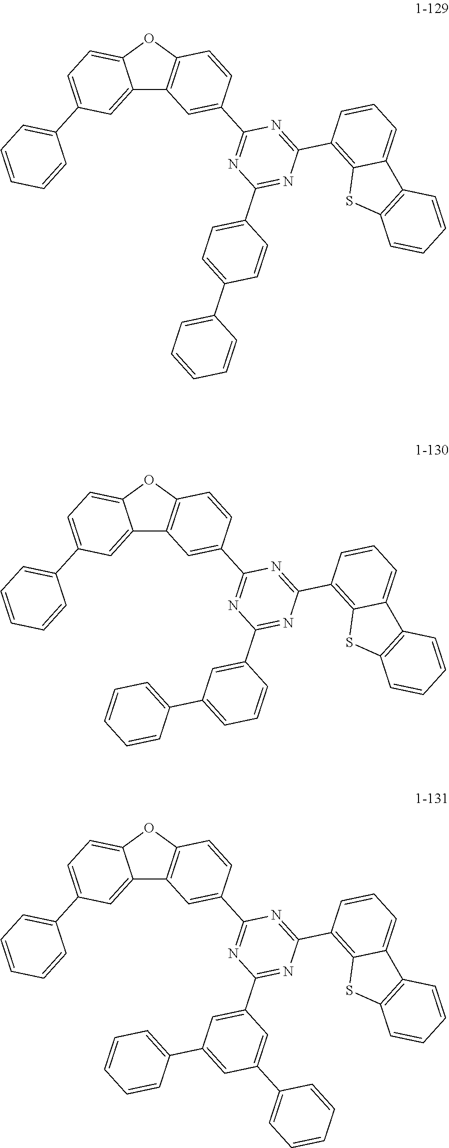 [SMILES: c1ccc(-c2ccc(-c3nc(-c4ccc5oc6ccc(-c7ccccc7)cc6c5c4)nc(-c4cccc5c4sc4ccccc45)n3)cc2)cc1 ; c1ccc(-c2cccc(-c3nc(-c4ccc5oc6ccc(-c7ccccc7)cc6c5c4)nc(-c4cccc5c4sc4ccccc45)n3)c2)cc1 ; c1ccc(-c2cc(-c3ccccc3)cc(-c3nc(-c4ccc5oc6ccc(-c7ccccc7)cc6c5c4)nc(-c4cccc5c4sc4ccccc45)n3)c2)cc1]

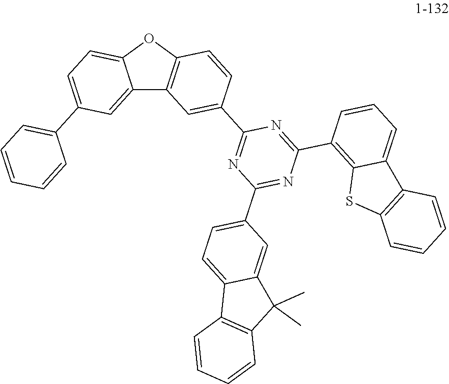 [SMILES: CC1(C)c2ccccc2-c2ccc(-c3nc(-c4ccc5oc6ccc(-c7ccccc7)cc6c5c4)nc(-c4cccc5c4sc4ccccc45)n3)cc21]

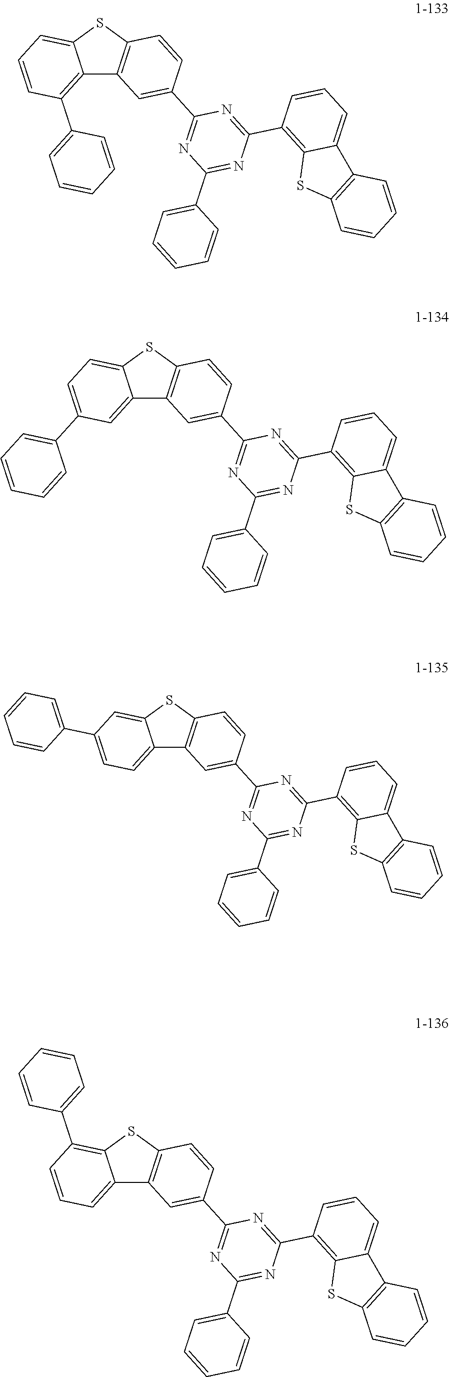 [SMILES: c1ccc(-c2nc(-c3ccc4sc5cccc(-c6ccccc6)c5c4c3)nc(-c3cccc4c3sc3ccccc34)n2)cc1 ; c1ccc(-c2ccc3sc4ccc(-c5nc(-c6ccccc6)nc(-c6cccc7c6sc6ccccc67)n5)cc4c3c2)cc1 ; c1ccc(-c2ccc3c(c2)sc2ccc(-c4nc(-c5ccccc5)nc(-c5cccc6c5sc5ccccc56)n4)cc23)cc1 ; c1ccc(-c2nc(-c3ccc4sc5c(-c6ccccc6)cccc5c4c3)nc(-c3cccc4c3sc3ccccc34)n2)cc1]

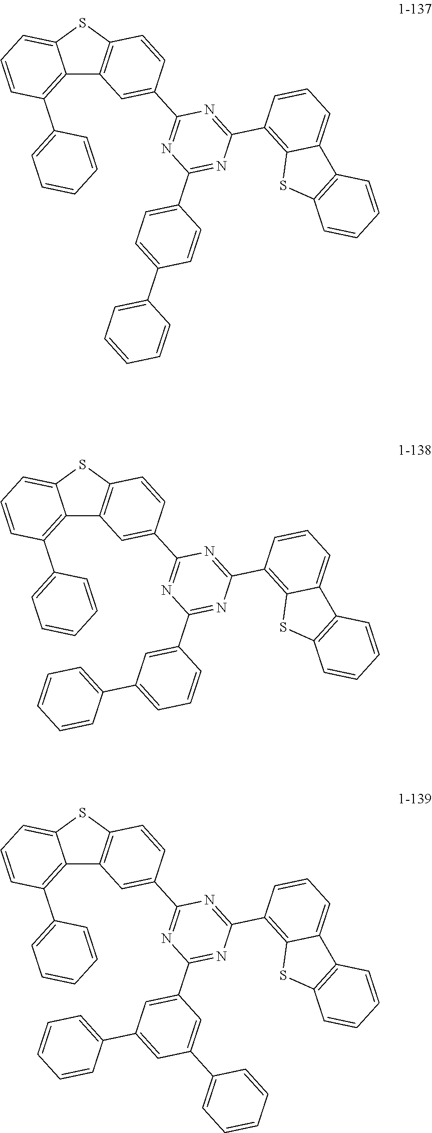 [SMILES: c1ccc(-c2ccc(-c3nc(-c4ccc5sc6cccc(-c7ccccc7)c6c5c4)nc(-c4cccc5c4sc4ccccc45)n3)cc2)cc1 ; c1ccc(-c2cccc(-c3nc(-c4ccc5sc6cccc(-c7ccccc7)c6c5c4)nc(-c4cccc5c4sc4ccccc45)n3)c2)cc1 ; c1ccc(-c2cc(-c3ccccc3)cc(-c3nc(-c4ccc5sc6cccc(-c7ccccc7)c6c5c4)nc(-c4cccc5c4sc4ccccc45)n3)c2)cc1]

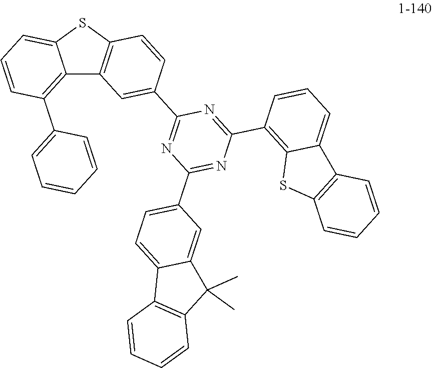 [SMILES: CC1(C)c2ccccc2-c2ccc(-c3nc(-c4ccc5sc6cccc(-c7ccccc7)c6c5c4)nc(-c4cccc5c4sc4ccccc45)n3)cc21]

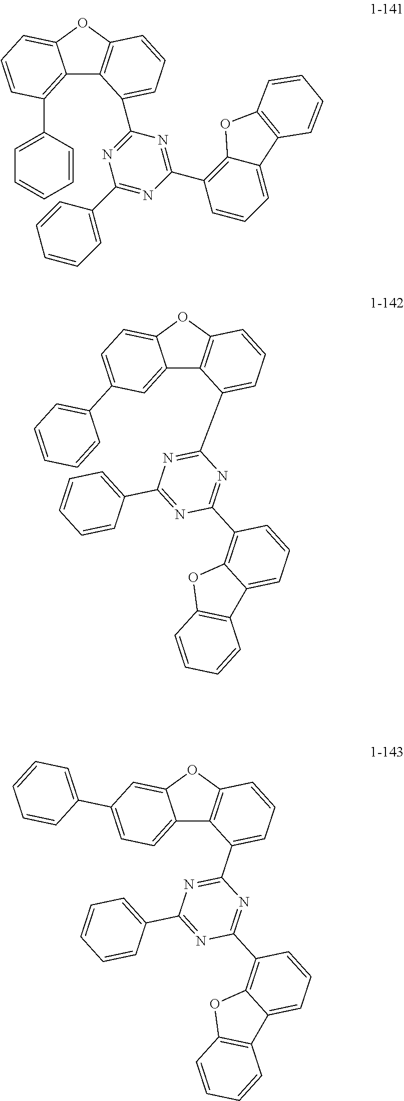 [SMILES: c1ccc(-c2nc(-c3cccc4c3oc3ccccc34)nc(-c3cccc4oc5cccc(-c6ccccc6)c5c34)n2)cc1 ; c1ccc(-c2ccc3oc4cccc(-c5nc(-c6ccccc6)nc(-c6cccc7c6oc6ccccc67)n5)c4c3c2)cc1 ; c1ccc(-c2ccc3c(c2)oc2cccc(-c4nc(-c5ccccc5)nc(-c5cccc6c5oc5ccccc56)n4)c23)cc1]

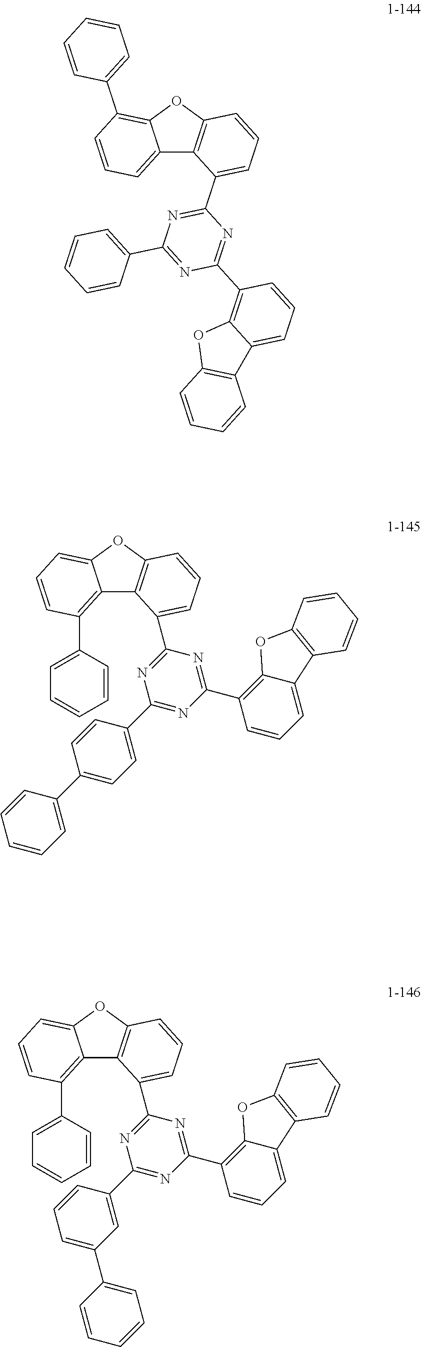 [SMILES: c1ccc(-c2nc(-c3cccc4c3oc3ccccc34)nc(-c3cccc4oc5c(-c6ccccc6)cccc5c34)n2)cc1 ; c1ccc(-c2ccc(-c3nc(-c4cccc5c4oc4ccccc45)nc(-c4cccc5oc6cccc(-c7ccccc7)c6c45)n3)cc2)cc1 ; c1ccc(-c2cccc(-c3nc(-c4cccc5c4oc4ccccc45)nc(-c4cccc5oc6cccc(-c7ccccc7)c6c45)n3)c2)cc1]

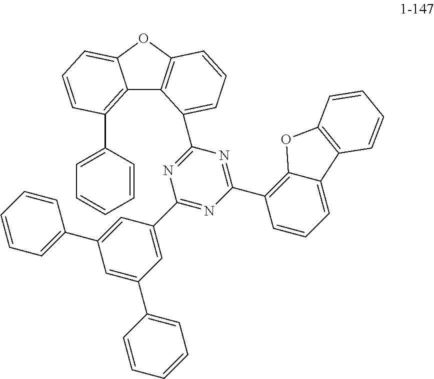 [SMILES: c1ccc(-c2cc(-c3ccccc3)cc(-c3nc(-c4cccc5c4oc4ccccc45)nc(-c4cccc5oc6cccc(-c7ccccc7)c6c45)n3)c2)cc1]

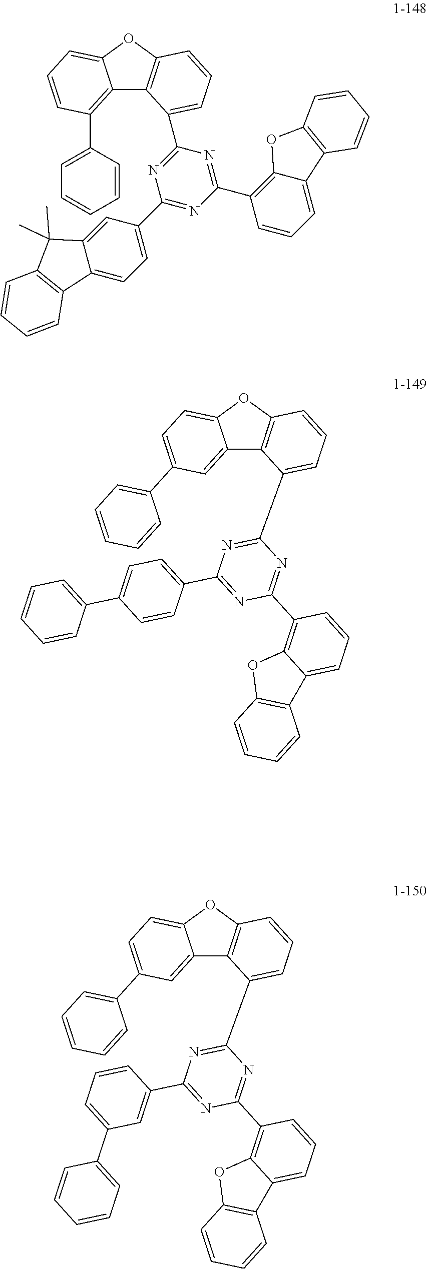 [SMILES: CC1(C)c2ccccc2-c2ccc(-c3nc(-c4cccc5c4oc4ccccc45)nc(-c4cccc5oc6cccc(-c7ccccc7)c6c45)n3)cc21 ; c1ccc(-c2ccc(-c3nc(-c4cccc5c4oc4ccccc45)nc(-c4cccc5oc6ccc(-c7ccccc7)cc6c45)n3)cc2)cc1 ; c1ccc(-c2cccc(-c3nc(-c4cccc5c4oc4ccccc45)nc(-c4cccc5oc6ccc(-c7ccccc7)cc6c45)n3)c2)cc1]

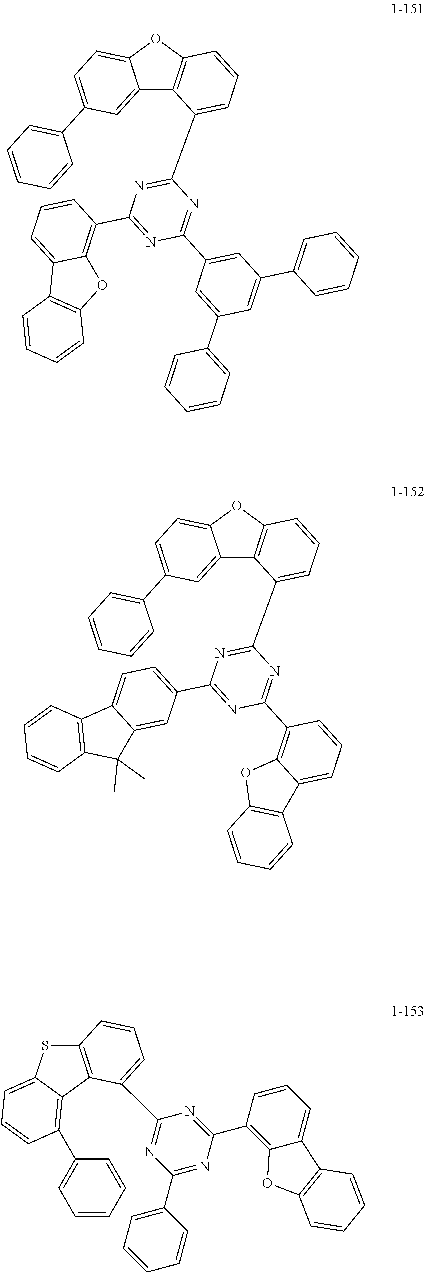 [SMILES: c1ccc(-c2cc(-c3ccccc3)cc(-c3nc(-c4cccc5c4oc4ccccc45)nc(-c4cccc5oc6ccc(-c7ccccc7)cc6c45)n3)c2)cc1 ; CC1(C)c2ccccc2-c2ccc(-c3nc(-c4cccc5c4oc4ccccc45)nc(-c4cccc5oc6ccc(-c7ccccc7)cc6c45)n3)cc21 ; c1ccc(-c2nc(-c3cccc4c3oc3ccccc34)nc(-c3cccc4sc5cccc(-c6ccccc6)c5c34)n2)cc1]

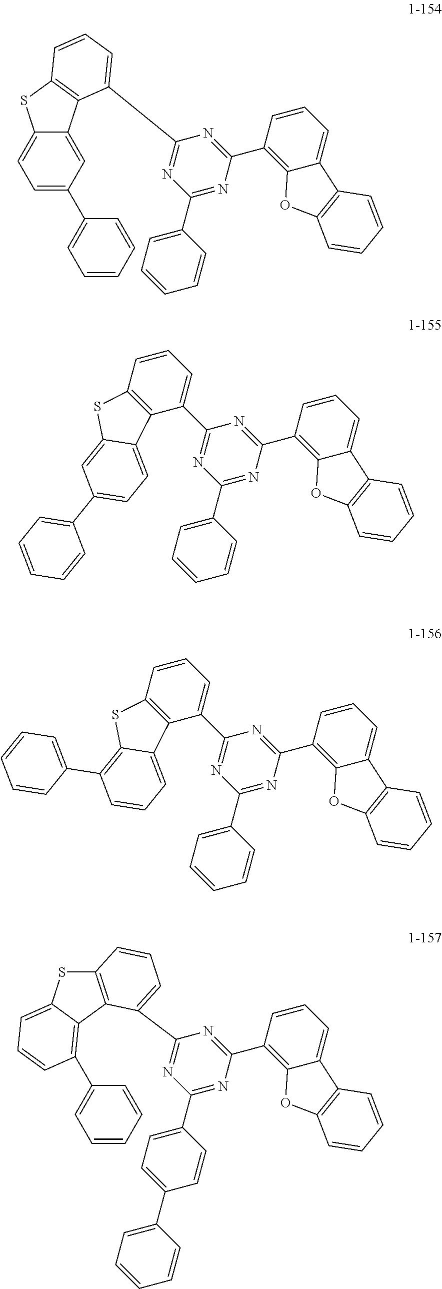 [SMILES: c1ccc(-c2ccc3sc4cccc(-c5nc(-c6ccccc6)nc(-c6cccc7c6oc6ccccc67)n5)c4c3c2)cc1 ; c1ccc(-c2ccc3c(c2)sc2cccc(-c4nc(-c5ccccc5)nc(-c5cccc6c5oc5ccccc56)n4)c23)cc1 ; c1ccc(-c2nc(-c3cccc4c3oc3ccccc34)nc(-c3cccc4sc5c(-c6ccccc6)cccc5c34)n2)cc1 ; c1ccc(-c2ccc(-c3nc(-c4cccc5c4oc4ccccc45)nc(-c4cccc5sc6cccc(-c7ccccc7)c6c45)n3)cc2)cc1]

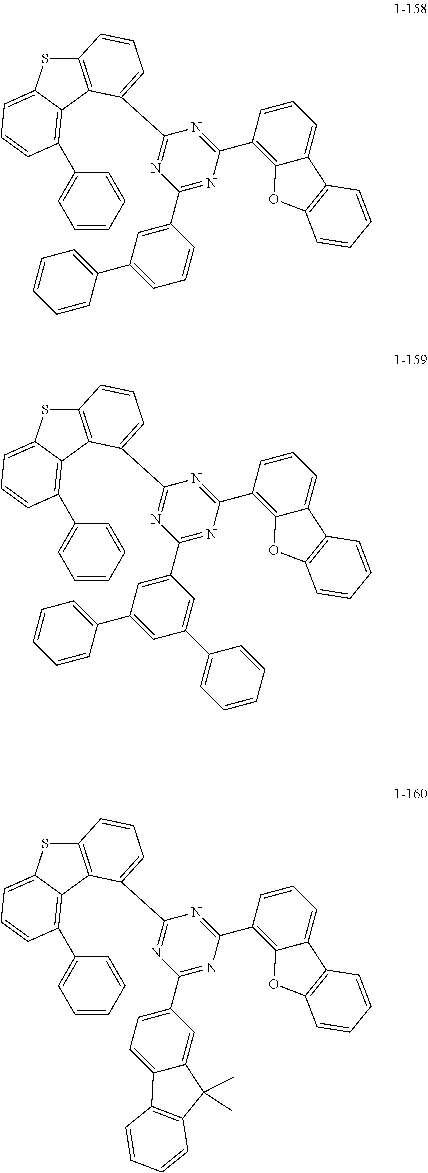 [SMILES: c1ccc(-c2cccc(-c3nc(-c4cccc5c4oc4ccccc45)nc(-c4cccc5sc6cccc(-c7ccccc7)c6c45)n3)c2)cc1 ; c1ccc(-c2cc(-c3ccccc3)cc(-c3nc(-c4cccc5c4oc4ccccc45)nc(-c4cccc5sc6cccc(-c7ccccc7)c6c45)n3)c2)cc1 ; CC1(C)c2ccccc2-c2ccc(-c3nc(-c4cccc5c4oc4ccccc45)nc(-c4cccc5sc6cccc(-c7ccccc7)c6c45)n3)cc21]

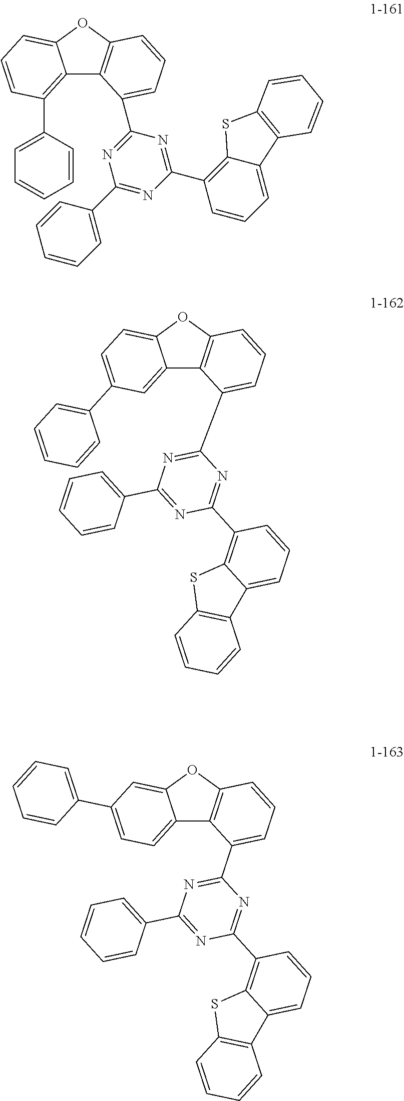 [SMILES: c1ccc(-c2nc(-c3cccc4c3sc3ccccc34)nc(-c3cccc4oc5cccc(-c6ccccc6)c5c34)n2)cc1 ; c1ccc(-c2ccc3oc4cccc(-c5nc(-c6ccccc6)nc(-c6cccc7c6sc6ccccc67)n5)c4c3c2)cc1 ; c1ccc(-c2ccc3c(c2)oc2cccc(-c4nc(-c5ccccc5)nc(-c5cccc6c5sc5ccccc56)n4)c23)cc1]

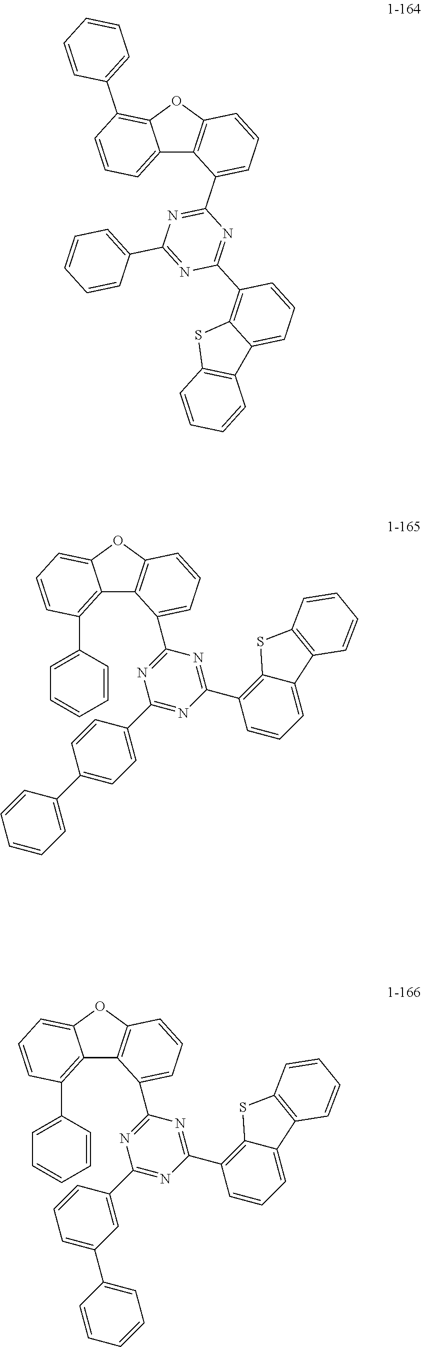 [SMILES: c1ccc(-c2nc(-c3cccc4c3sc3ccccc34)nc(-c3cccc4oc5c(-c6ccccc6)cccc5c34)n2)cc1 ; c1ccc(-c2ccc(-c3nc(-c4cccc5c4sc4ccccc45)nc(-c4cccc5oc6cccc(-c7ccccc7)c6c45)n3)cc2)cc1 ; c1ccc(-c2cccc(-c3nc(-c4cccc5c4sc4ccccc45)nc(-c4cccc5oc6cccc(-c7ccccc7)c6c45)n3)c2)cc1]

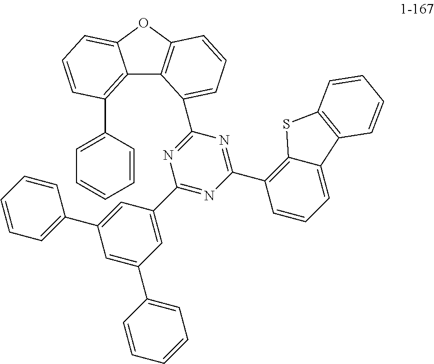 [SMILES: c1ccc(-c2cc(-c3ccccc3)cc(-c3nc(-c4cccc5c4sc4ccccc45)nc(-c4cccc5oc6cccc(-c7ccccc7)c6c45)n3)c2)cc1]

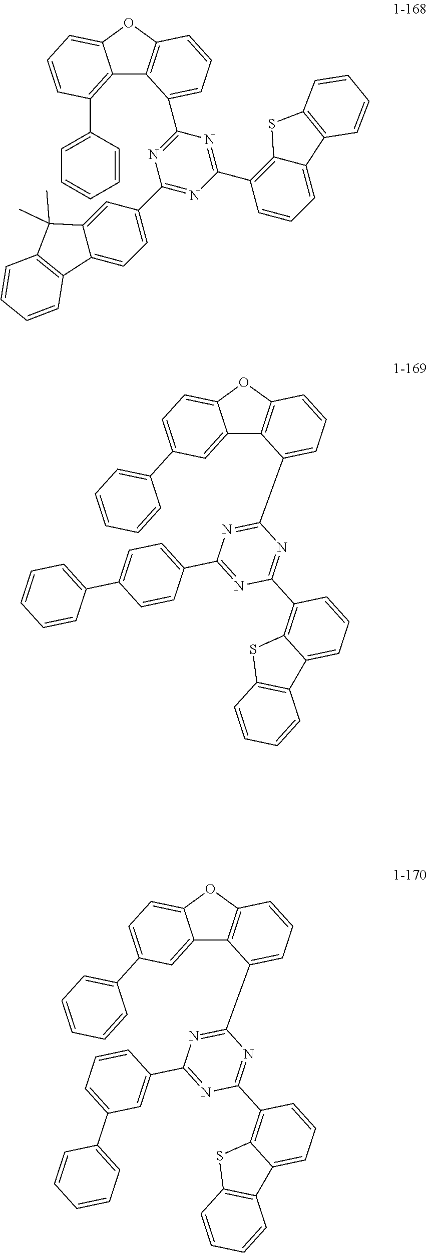 [SMILES: CC1(C)c2ccccc2-c2ccc(-c3nc(-c4cccc5c4sc4ccccc45)nc(-c4cccc5oc6cccc(-c7ccccc7)c6c45)n3)cc21 ; c1ccc(-c2ccc(-c3nc(-c4cccc5c4sc4ccccc45)nc(-c4cccc5oc6ccc(-c7ccccc7)cc6c45)n3)cc2)cc1 ; c1ccc(-c2cccc(-c3nc(-c4cccc5c4sc4ccccc45)nc(-c4cccc5oc6ccc(-c7ccccc7)cc6c45)n3)c2)cc1]

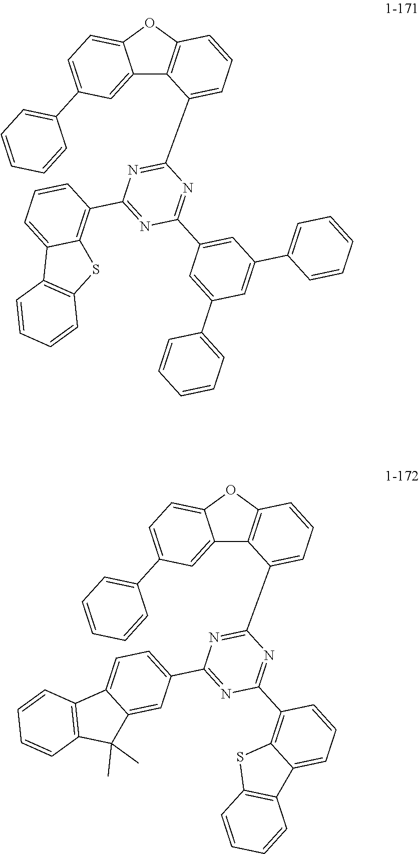 [SMILES: c1ccc(-c2cc(-c3ccccc3)cc(-c3nc(-c4cccc5c4sc4ccccc45)nc(-c4cccc5oc6ccc(-c7ccccc7)cc6c45)n3)c2)cc1 ; CC1(C)c2ccccc2-c2ccc(-c3nc(-c4cccc5c4sc4ccccc45)nc(-c4cccc5oc6ccc(-c7ccccc7)cc6c45)n3)cc21]

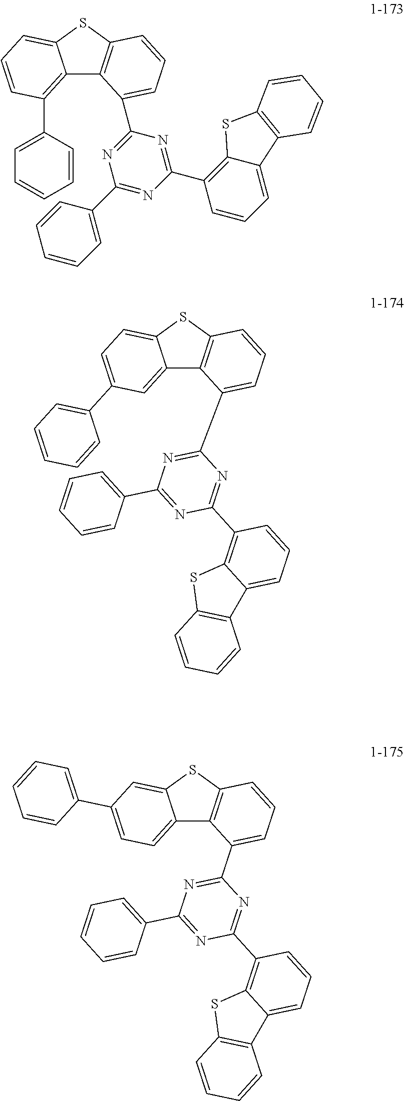 [SMILES: c1ccc(-c2nc(-c3cccc4c3sc3ccccc34)nc(-c3cccc4sc5cccc(-c6ccccc6)c5c34)n2)cc1 ; c1ccc(-c2ccc3sc4cccc(-c5nc(-c6ccccc6)nc(-c6cccc7c6sc6ccccc67)n5)c4c3c2)cc1 ; c1ccc(-c2ccc3c(c2)sc2cccc(-c4nc(-c5ccccc5)nc(-c5cccc6c5sc5ccccc56)n4)c23)cc1]

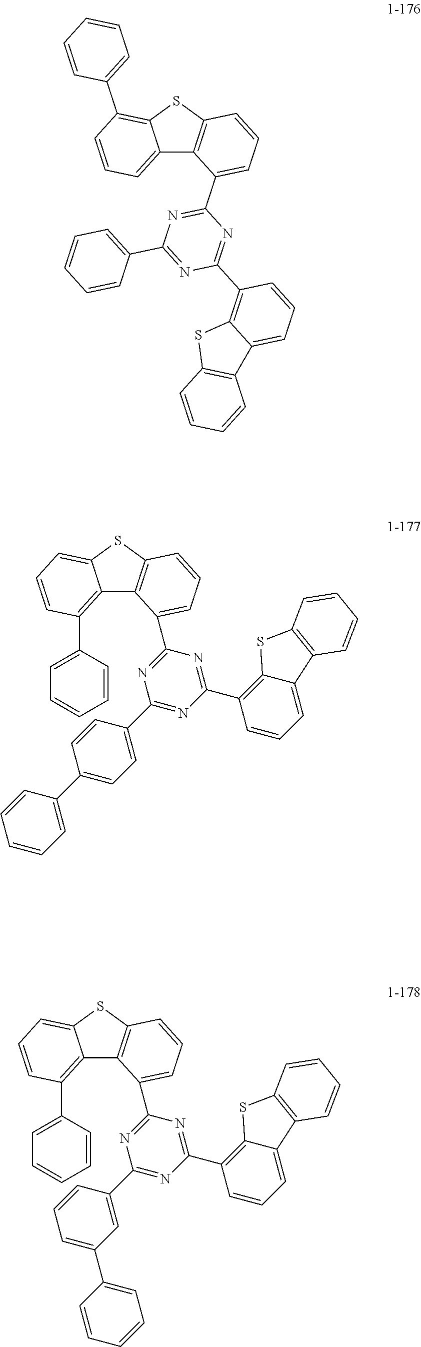 [SMILES: c1ccc(-c2nc(-c3cccc4c3sc3ccccc34)nc(-c3cccc4sc5c(-c6ccccc6)cccc5c34)n2)cc1 ; c1ccc(-c2ccc(-c3nc(-c4cccc5c4sc4ccccc45)nc(-c4cccc5sc6cccc(-c7ccccc7)c6c45)n3)cc2)cc1 ; c1ccc(-c2cccc(-c3nc(-c4cccc5c4sc4ccccc45)nc(-c4cccc5sc6cccc(-c7ccccc7)c6c45)n3)c2)cc1]

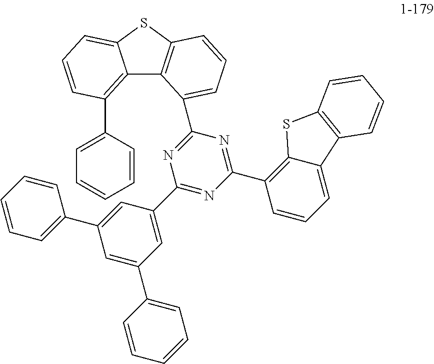 [SMILES: c1ccc(-c2cc(-c3ccccc3)cc(-c3nc(-c4cccc5c4sc4ccccc45)nc(-c4cccc5sc6cccc(-c7ccccc7)c6c45)n3)c2)cc1]

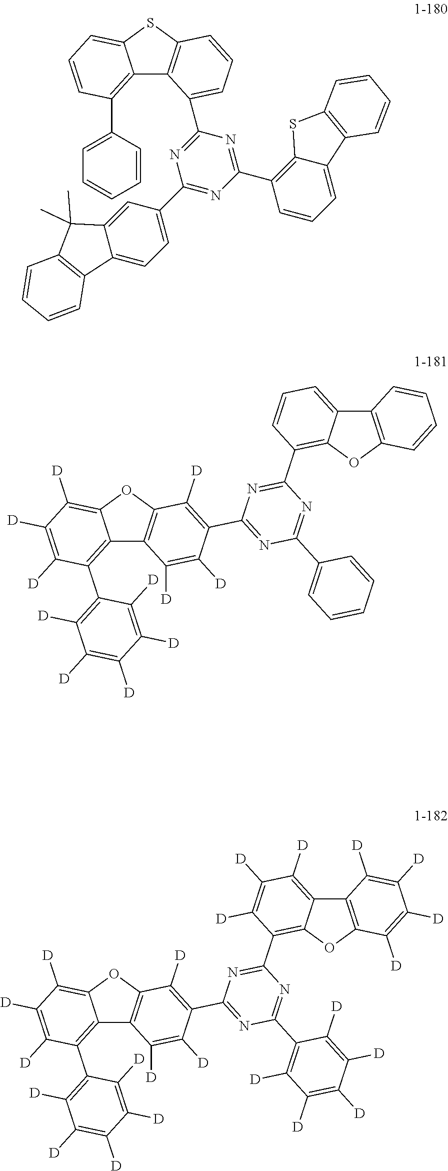 [SMILES: CC1(C)c2ccccc2-c2ccc(-c3nc(-c4cccc5c4sc4ccccc45)nc(-c4cccc5sc6cccc(-c7ccccc7)c6c45)n3)cc21 ; [2H]c1c([2H])c([2H])c(-c2c([2H])c([2H])c([2H])c3oc4c([2H])c(-c5nc(-c6ccccc6)nc(-c6cccc7c6oc6ccccc67)n5)c([2H])c([2H])c4c23)c([2H])c1[2H] ; [2H]c1c([2H])c([2H])c(-c2nc(-c3c([2H])c([2H])c4c(oc5c([2H])c([2H])c([2H])c(-c6c([2H])c([2H])c([2H])c([2H])c6[2H])c54)c3[2H])nc(-c3c([2H])c([2H])c([2H])c4c3oc3c([2H])c([2H])c([2H])c([2H])c34)n2)c([2H])c1[2H]]

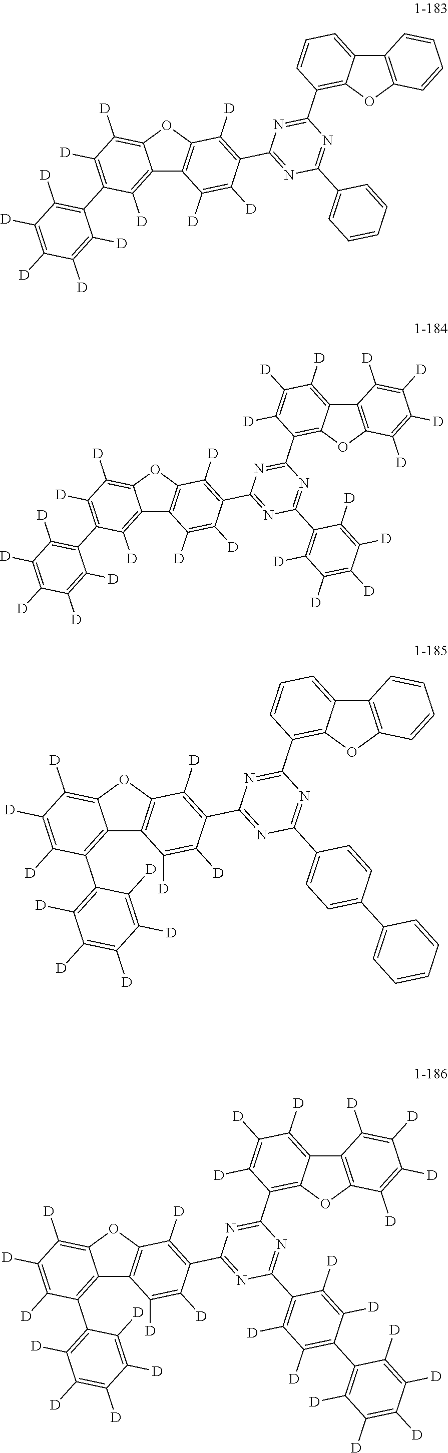

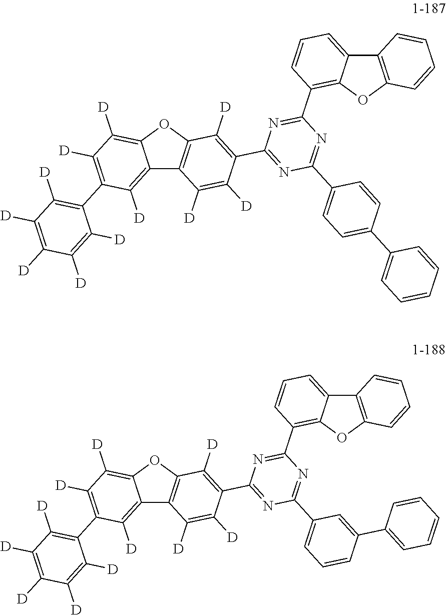 [SMILES: [2H]c1c([2H])c([2H])c(-c2c([2H])c([2H])c3oc4c([2H])c(-c5nc(-c6ccc(-c7ccccc7)cc6)nc(-c6cccc7c6oc6ccccc67)n5)c([2H])c([2H])c4c3c2[2H])c([2H])c1[2H] ; [2H]c1c([2H])c([2H])c(-c2c([2H])c([2H])c3oc4c([2H])c(-c5nc(-c6cccc(-c7ccccc7)c6)nc(-c6cccc7c6oc6ccccc67)n5)c([2H])c([2H])c4c3c2[2H])c([2H])c1[2H]]

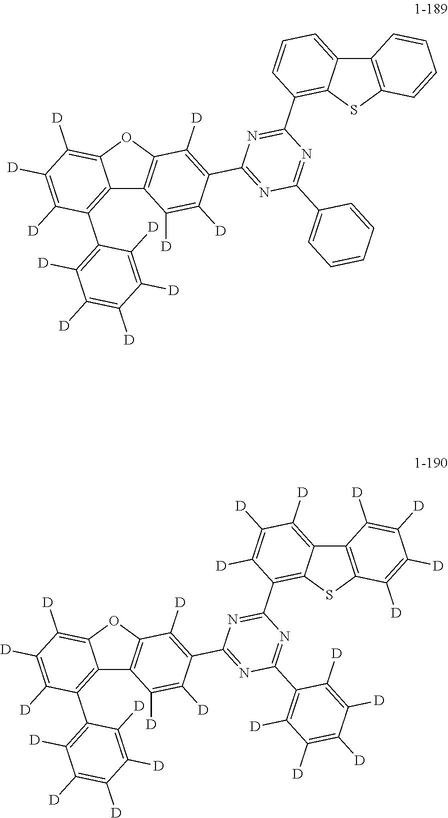 [SMILES: [2H]c1c([2H])c([2H])c(-c2c([2H])c([2H])c([2H])c3oc4c([2H])c(-c5nc(-c6ccccc6)nc(-c6cccc7c6sc6ccccc67)n5)c([2H])c([2H])c4c23)c([2H])c1[2H] ; [2H]c1c([2H])c([2H])c(-c2nc(-c3c([2H])c([2H])c4c(oc5c([2H])c([2H])c([2H])c(-c6c([2H])c([2H])c([2H])c([2H])c6[2H])c54)c3[2H])nc(-c3c([2H])c([2H])c([2H])c4c3sc3c([2H])c([2H])c([2H])c([2H])c34)n2)c([2H])c1[2H]]

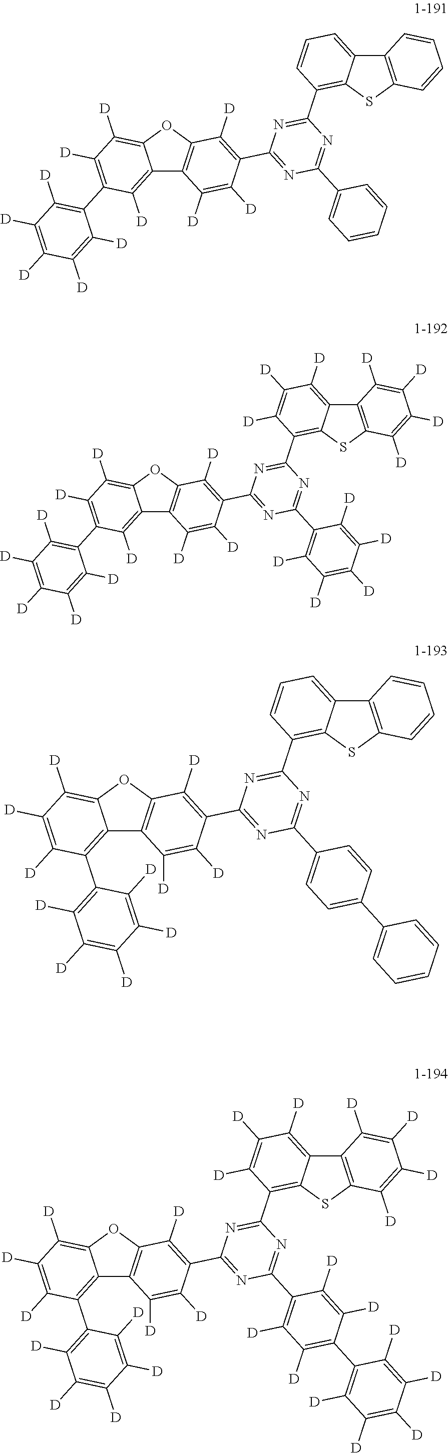

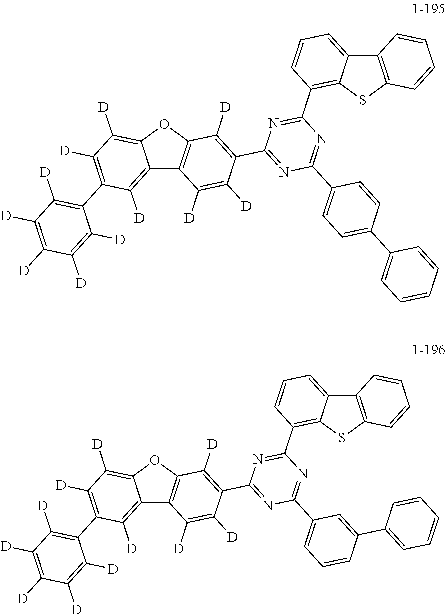 [SMILES: [2H]c1c([2H])c([2H])c(-c2c([2H])c([2H])c3oc4c([2H])c(-c5nc(-c6ccc(-c7ccccc7)cc6)nc(-c6cccc7c6sc6ccccc67)n5)c([2H])c([2H])c4c3c2[2H])c([2H])c1[2H] ; [2H]c1c([2H])c([2H])c(-c2c([2H])c([2H])c3oc4c([2H])c(-c5nc(-c6cccc(-c7ccccc7)c6)nc(-c6cccc7c6sc6ccccc67)n5)c([2H])c([2H])c4c3c2[2H])c([2H])c1[2H]]

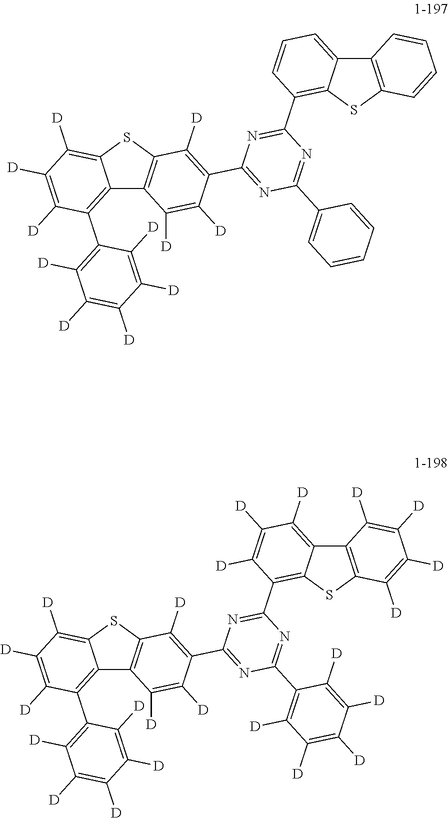 [SMILES: [2H]c1c([2H])c([2H])c(-c2c([2H])c([2H])c([2H])c3sc4c([2H])c(-c5nc(-c6ccccc6)nc(-c6cccc7c6sc6ccccc67)n5)c([2H])c([2H])c4c23)c([2H])c1[2H] ; [2H]c1c([2H])c([2H])c(-c2nc(-c3c([2H])c([2H])c4c(sc5c([2H])c([2H])c([2H])c(-c6c([2H])c([2H])c([2H])c([2H])c6[2H])c54)c3[2H])nc(-c3c([2H])c([2H])c([2H])c4c3sc3c([2H])c([2H])c([2H])c([2H])c34)n2)c([2H])c1[2H]]

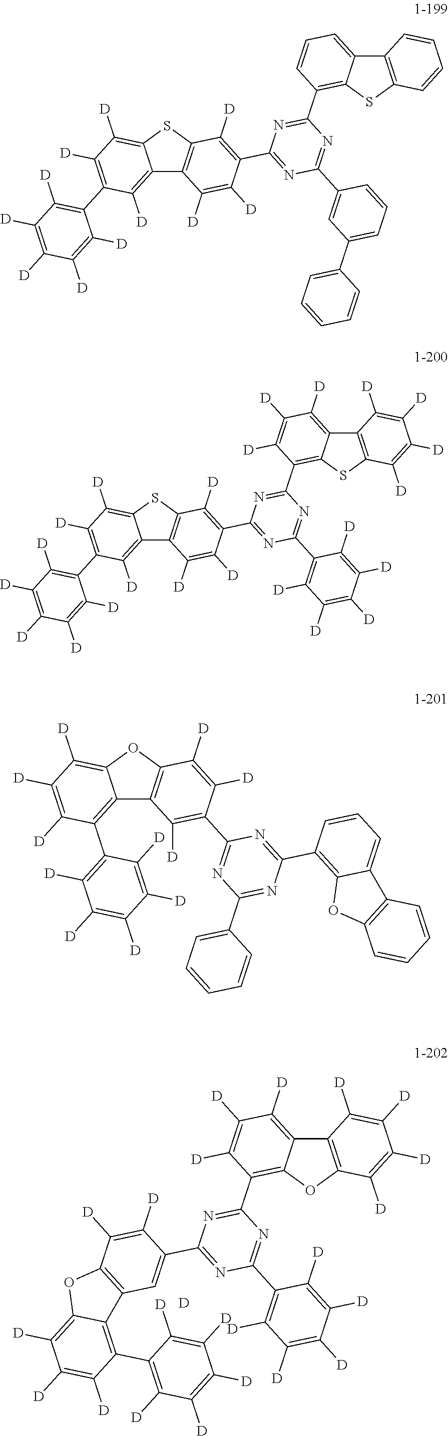

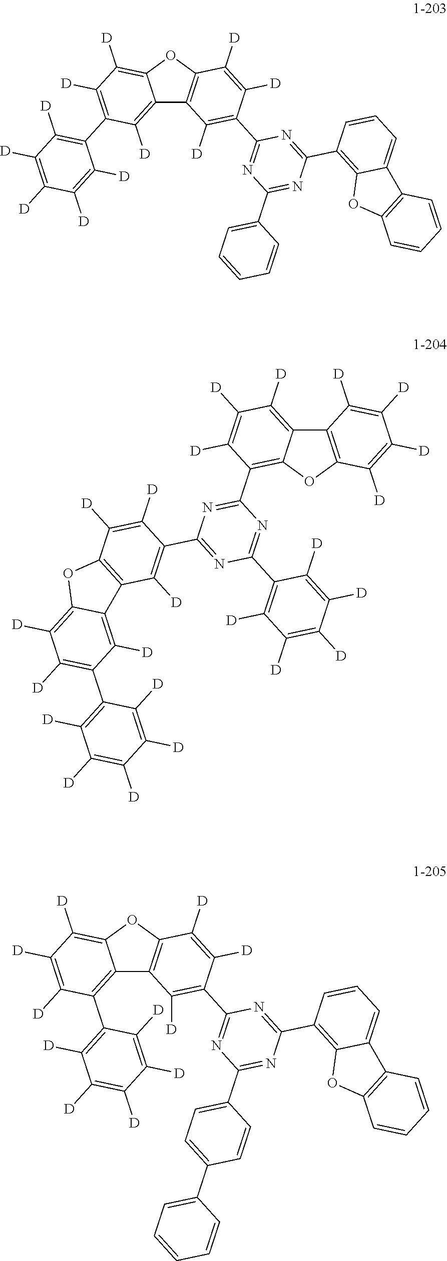 [SMILES: [2H]c1c([2H])c([2H])c(-c2c([2H])c([2H])c3oc4c([2H])c([2H])c(-c5nc(-c6ccccc6)nc(-c6cccc7c6oc6ccccc67)n5)c([2H])c4c3c2[2H])c([2H])c1[2H] ; [2H]c1c([2H])c([2H])c(-c2nc(-c3c([2H])c([2H])c4oc5c([2H])c([2H])c(-c6c([2H])c([2H])c([2H])c([2H])c6[2H])c([2H])c5c4c3[2H])nc(-c3c([2H])c([2H])c([2H])c4c3oc3c([2H])c([2H])c([2H])c([2H])c34)n2)c([2H])c1[2H] ; [2H]c1c([2H])c([2H])c(-c2c([2H])c([2H])c([2H])c3oc4c([2H])c([2H])c(-c5nc(-c6ccc(-c7ccccc7)cc6)nc(-c6cccc7c6oc6ccccc67)n5)c([2H])c4c23)c([2H])c1[2H]]

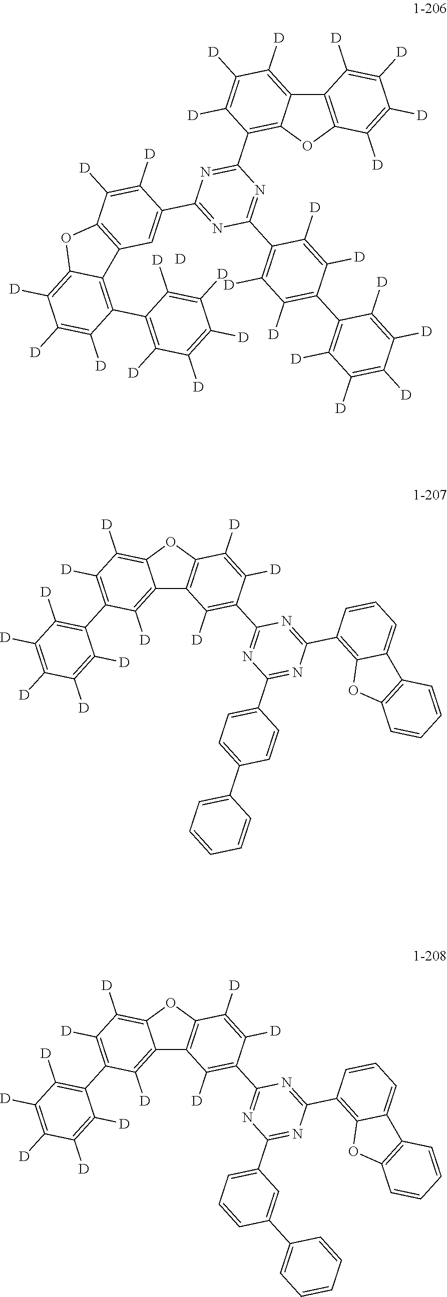

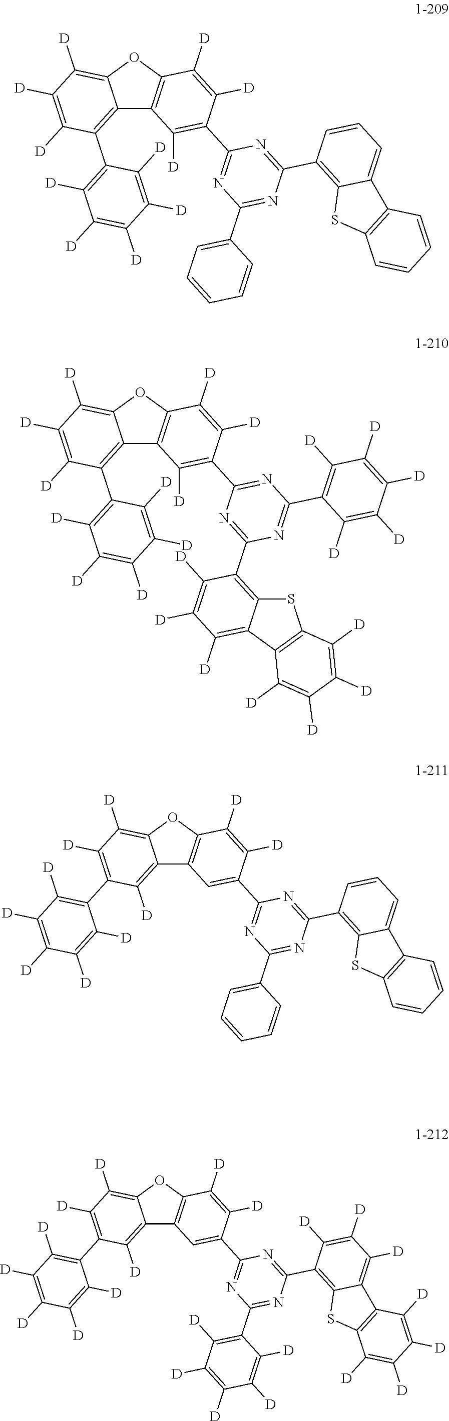

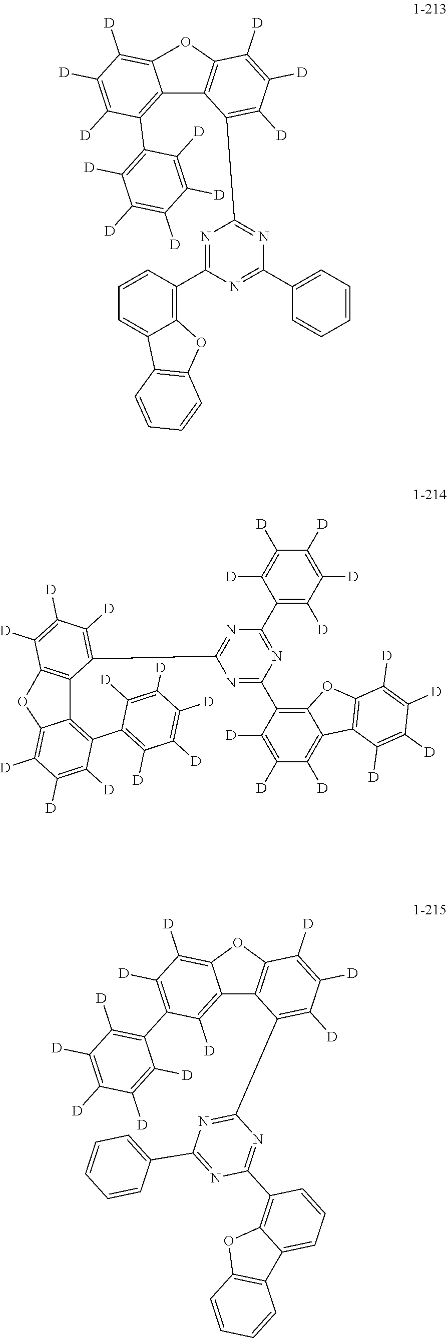 [SMILES: [2H]c1c([2H])c([2H])c(-c2c([2H])c([2H])c([2H])c3oc4c([2H])c([2H])c([2H])c(-c5nc(-c6ccccc6)nc(-c6cccc7c6oc6ccccc67)n5)c4c23)c([2H])c1[2H] ; [2H]c1c([2H])c([2H])c(-c2nc(-c3c([2H])c([2H])c([2H])c4c3oc3c([2H])c([2H])c([2H])c([2H])c34)nc(-c3c([2H])c([2H])c([2H])c4oc5c([2H])c([2H])c([2H])c(-c6c([2H])c([2H])c([2H])c([2H])c6[2H])c5c34)n2)c([2H])c1[2H] ; [2H]c1c([2H])c([2H])c(-c2c([2H])c([2H])c3oc4c([2H])c([2H])c([2H])c(-c5nc(-c6ccccc6)nc(-c6cccc7c6oc6ccccc67)n5)c4c3c2[2H])c([2H])c1[2H]]

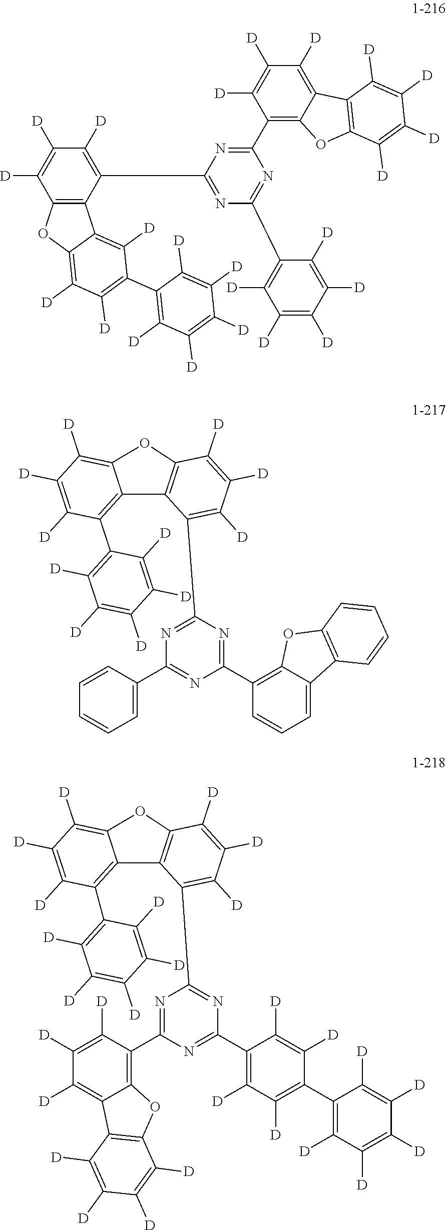

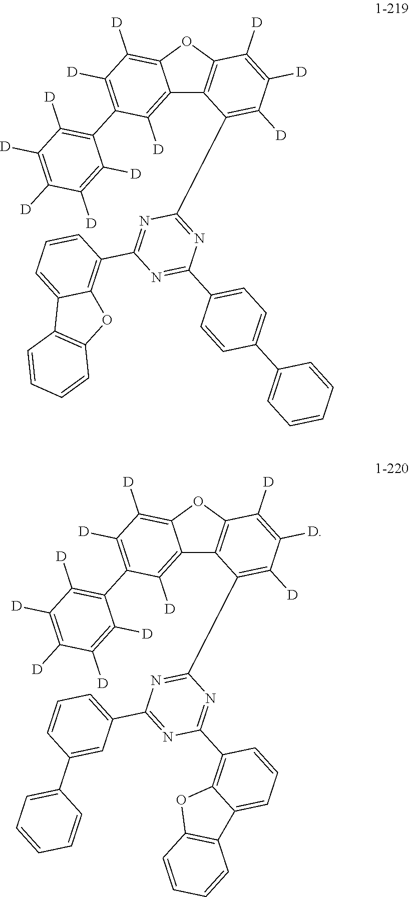

8. An organic light emitting device comprising:
a first electrode;
a second electrode provided to face the first electrode; and
an organic material layer having one or more layers provided between the first electrode and the second electrode,
wherein the one or more layers of the organic material layer comprise the heterocyclic compound of claim 1.

9. The organic light emitting device of claim 8, wherein the organic material layer further comprises a heterocyclic compound represented by the following Chemical Formula 2: 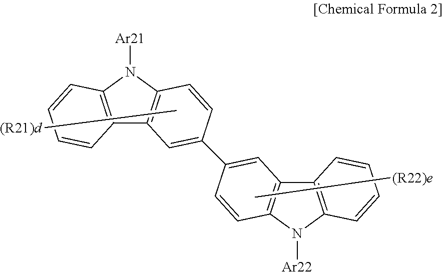
wherein, in Chemical Formula 2,
Ar21 and Ar22 are the same as or different from each other, and are each independently a substituted or unsubstituted C6 to C60 aryl group; or a substituted or unsubstituted C2 to C60 heteroaryl group,
R21 and R22 are the same as or different from each other, and are each independently selected from the group consisting of hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more adjacent groups are bonded to each other to form a substituted or unsubstituted aliphatic or aromatic hydrocarbon ring, and
d and e are an integer from 0 to 7, and when d and e are 2 or higher, substituents in the parenthesis are the same as or different from each other.

10. The organic light emitting device of claim 9, wherein a deuterium content of the heterocyclic compound represented by Chemical Formula 2 is 0% to 100%.

11. The organic light emitting device of claim 9, wherein R21 and R22 are the same as or different from each other, and are each independently hydrogen or deuterium.

12. The organic light emitting device of claim 9, wherein Chemical Formula 2 is represented by any one of the following compounds:

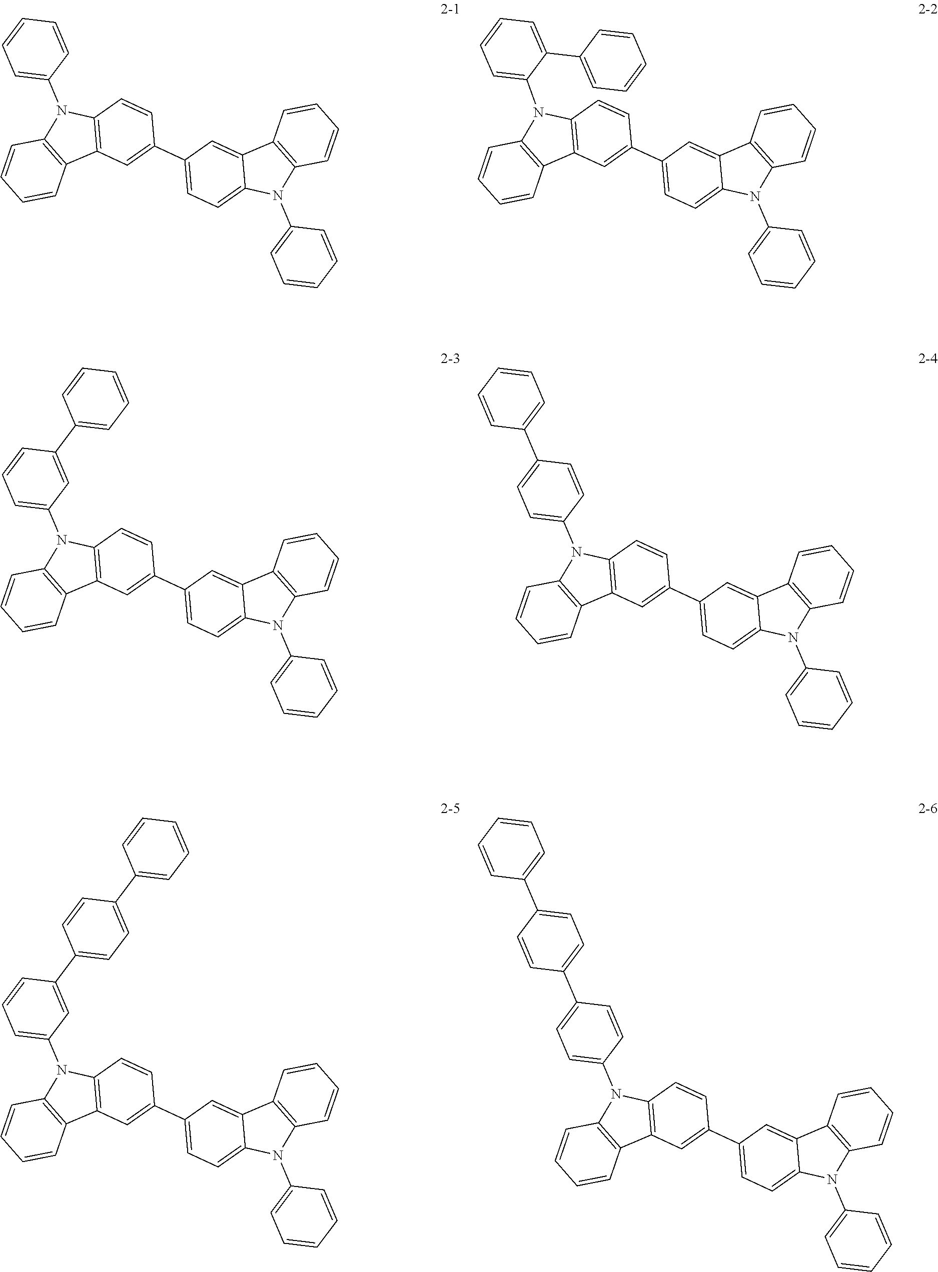 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

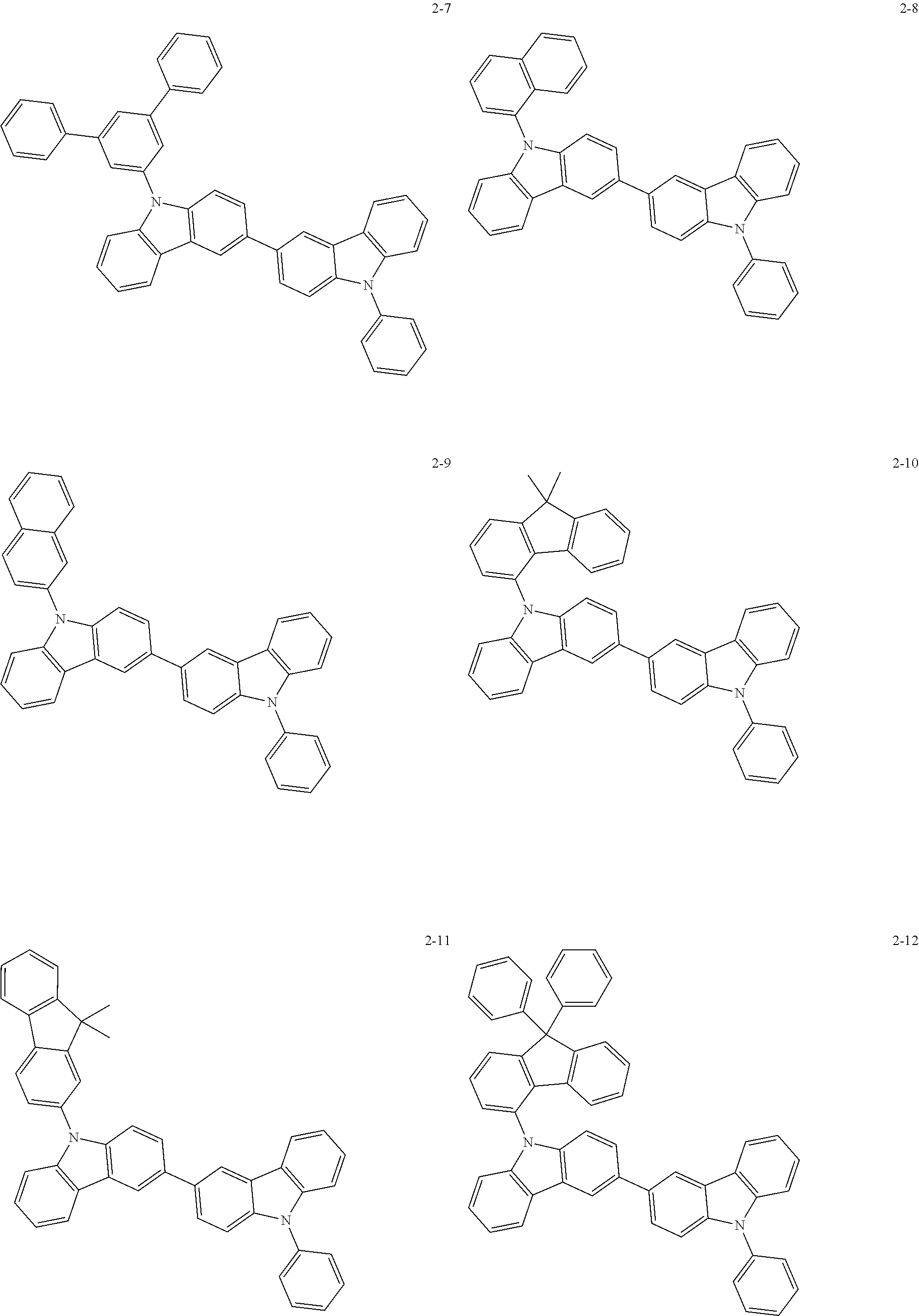

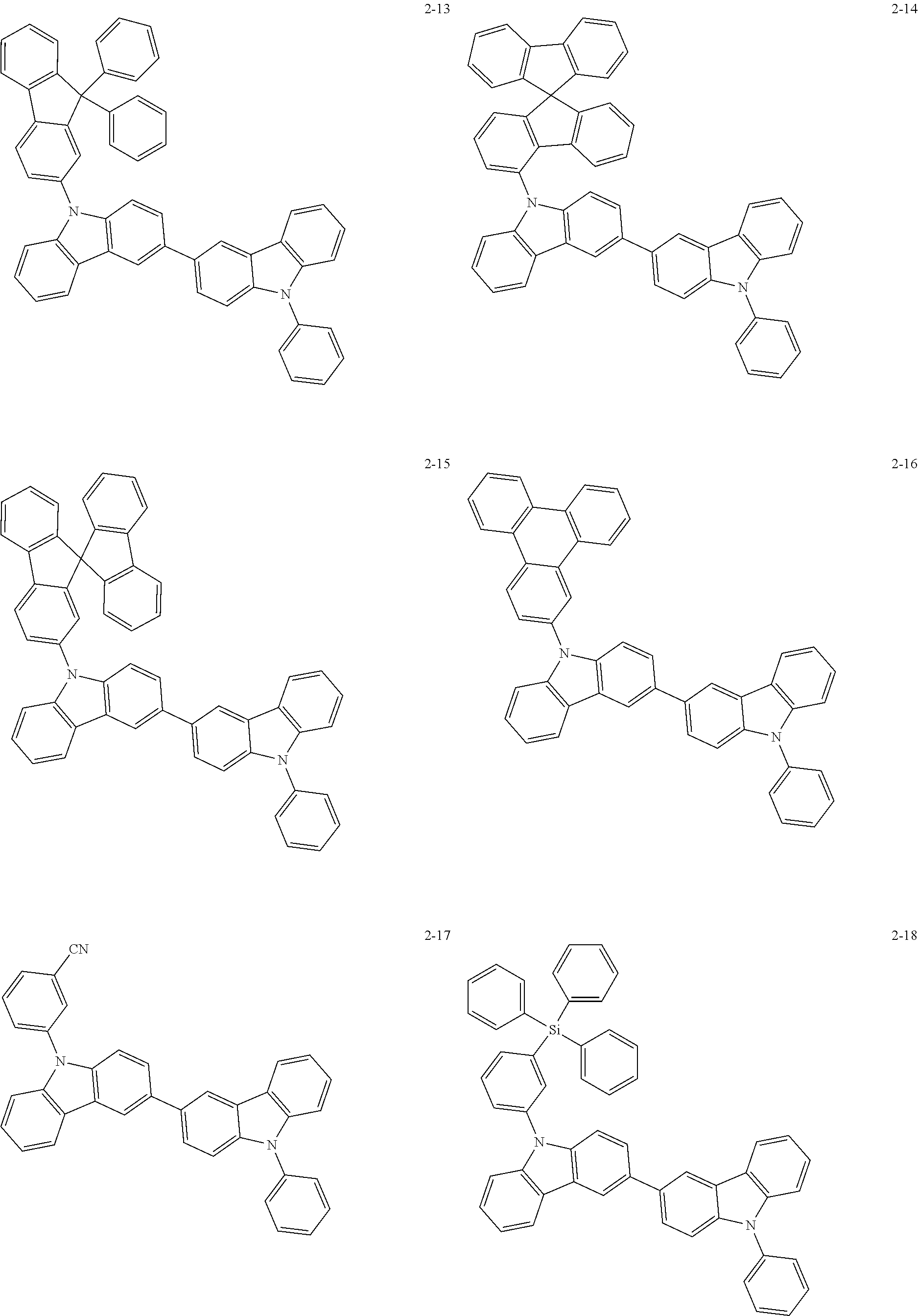

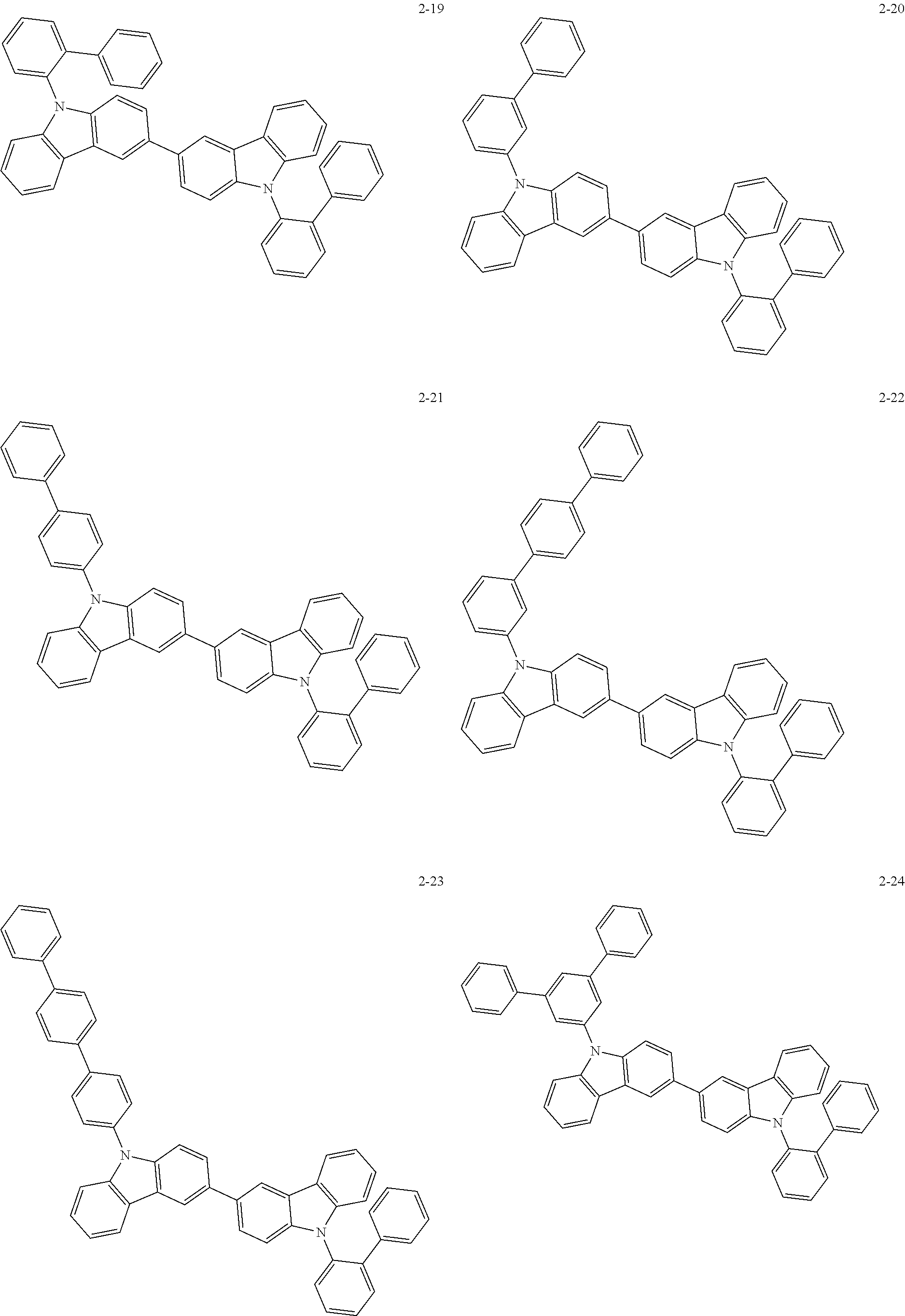

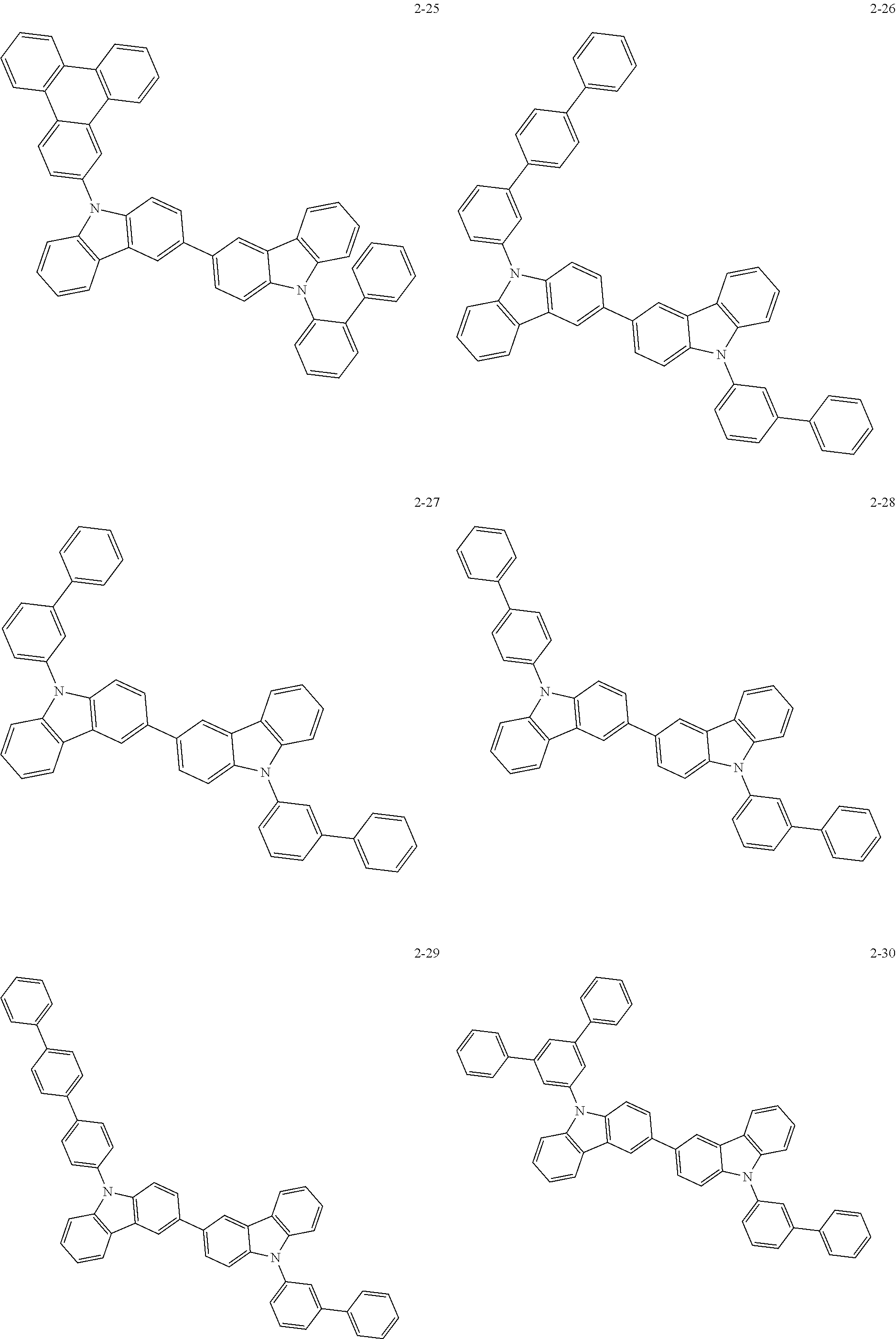

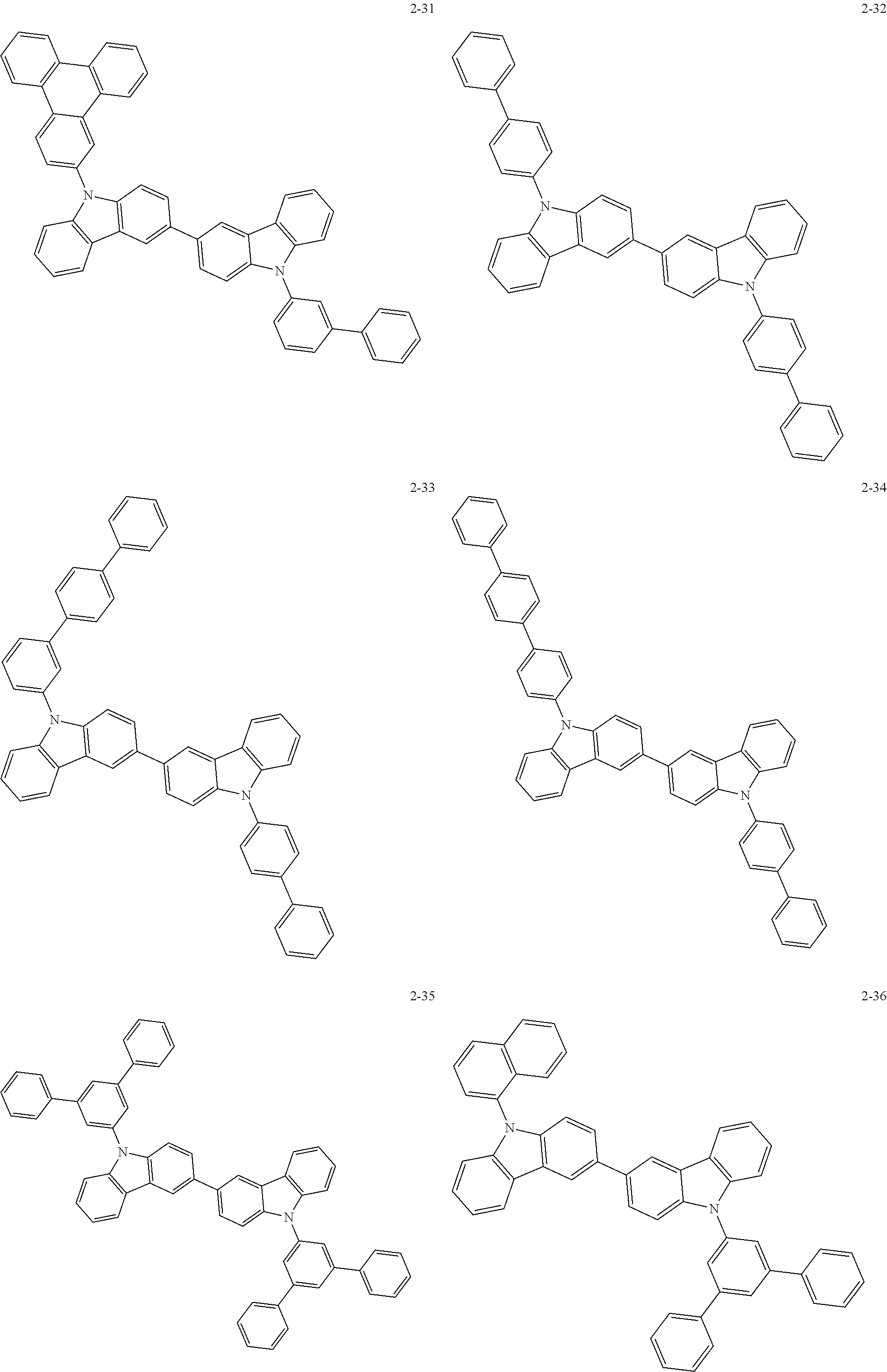

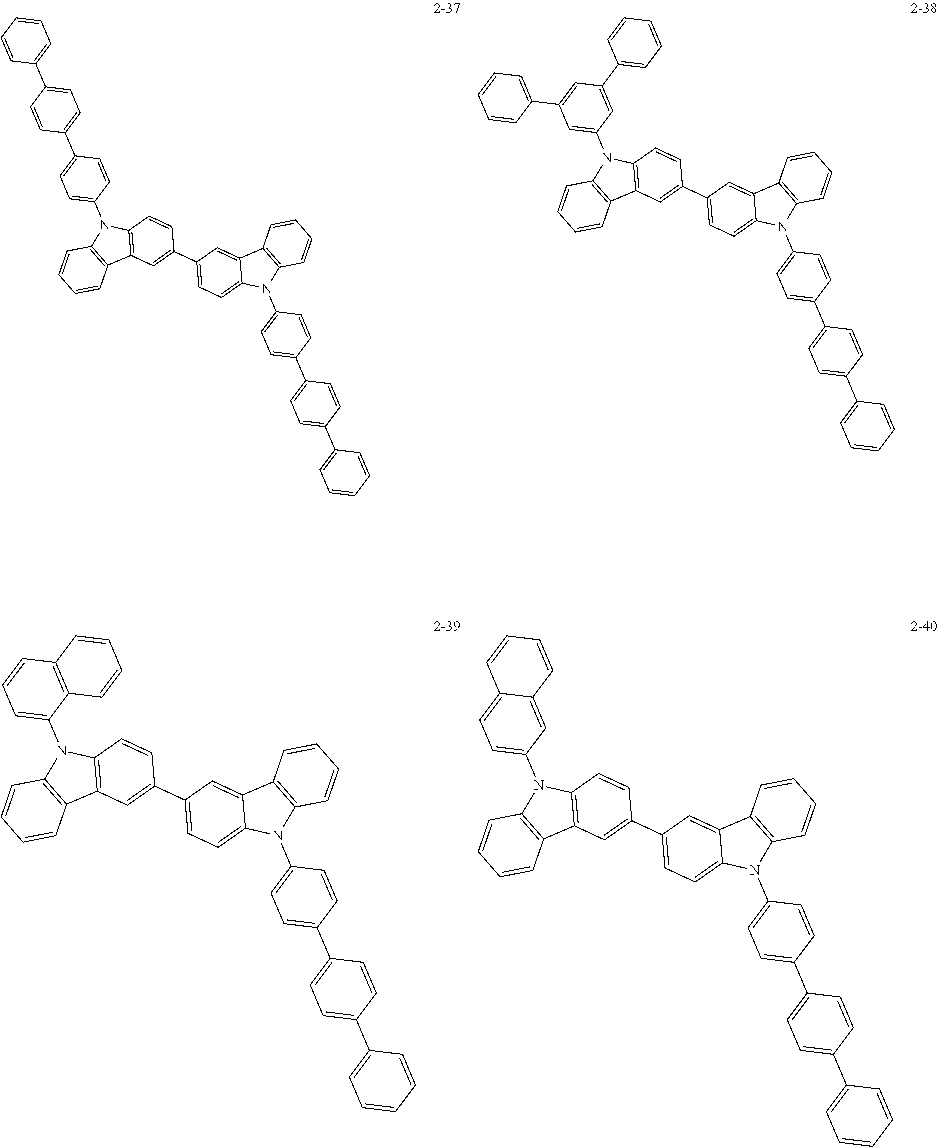 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

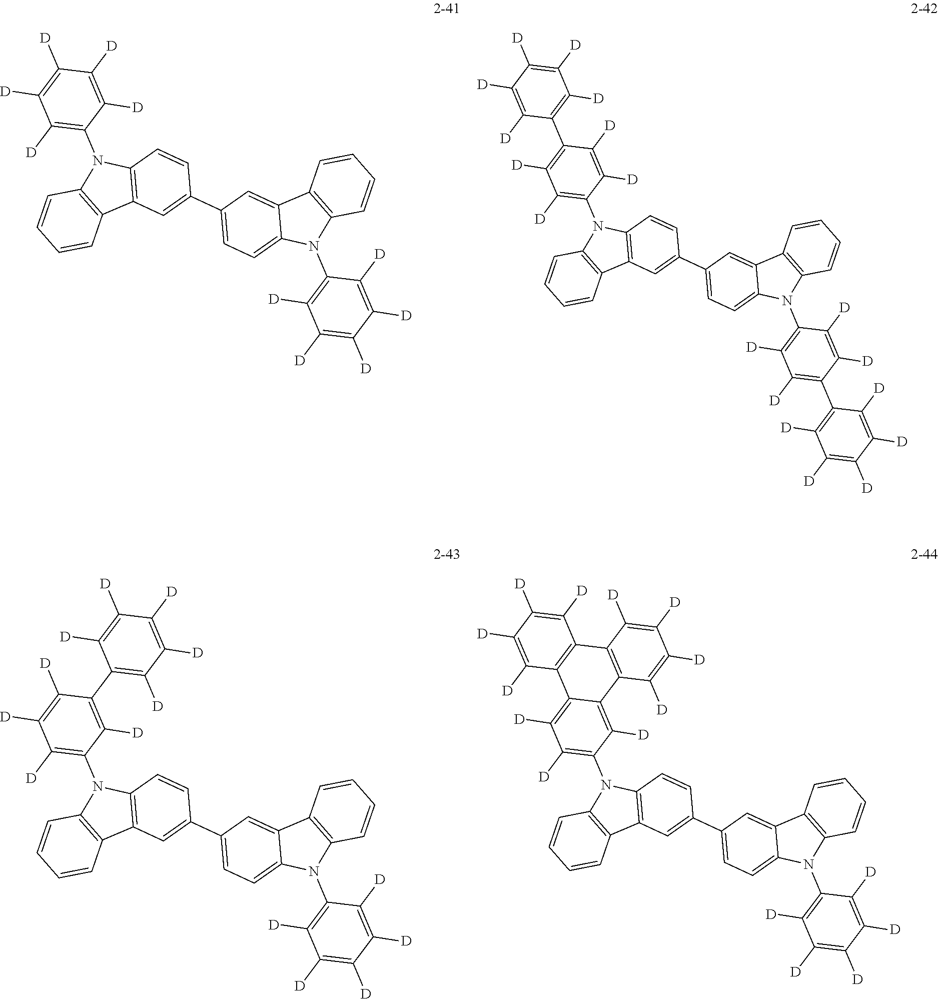

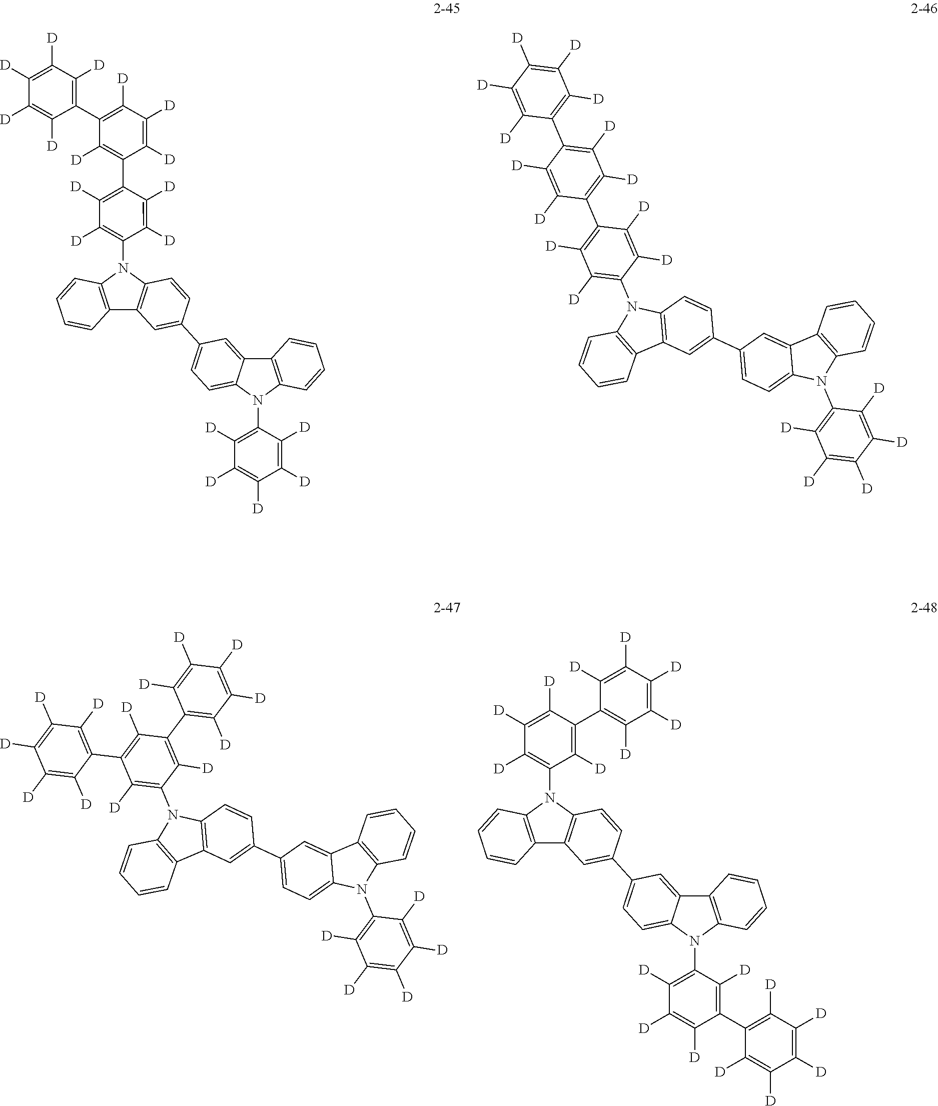

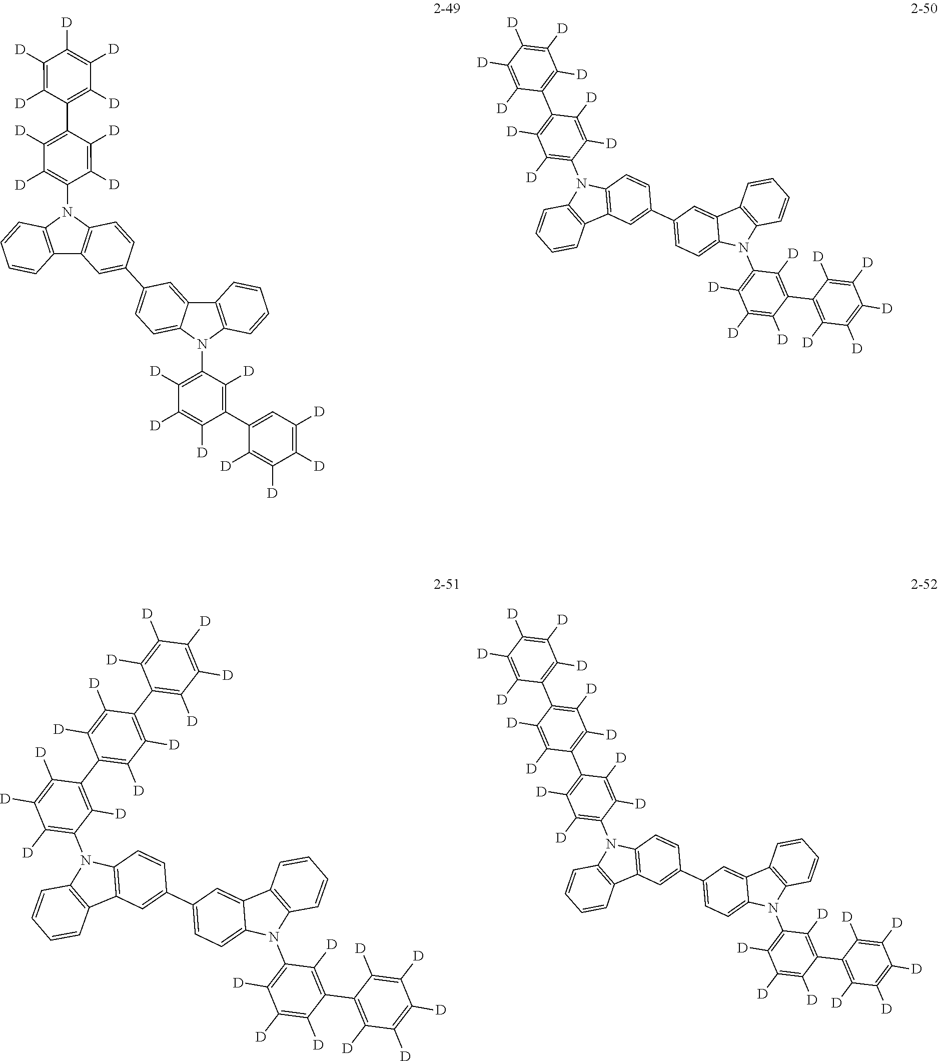

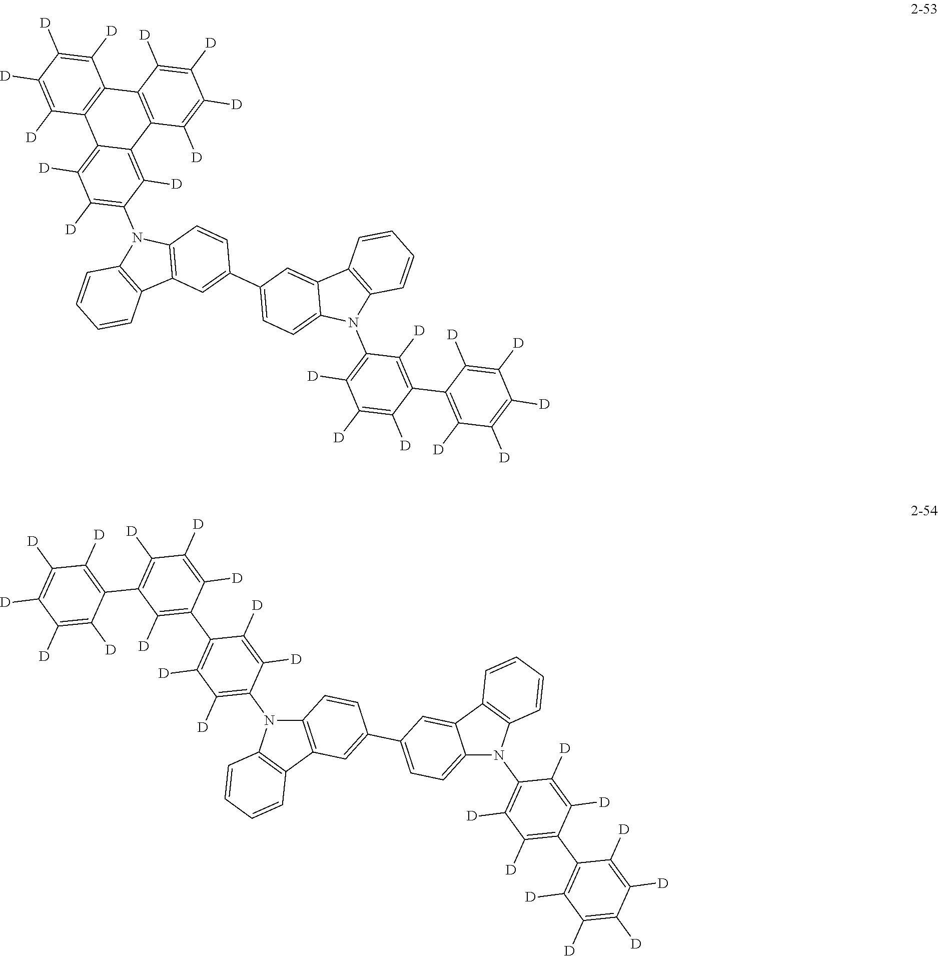

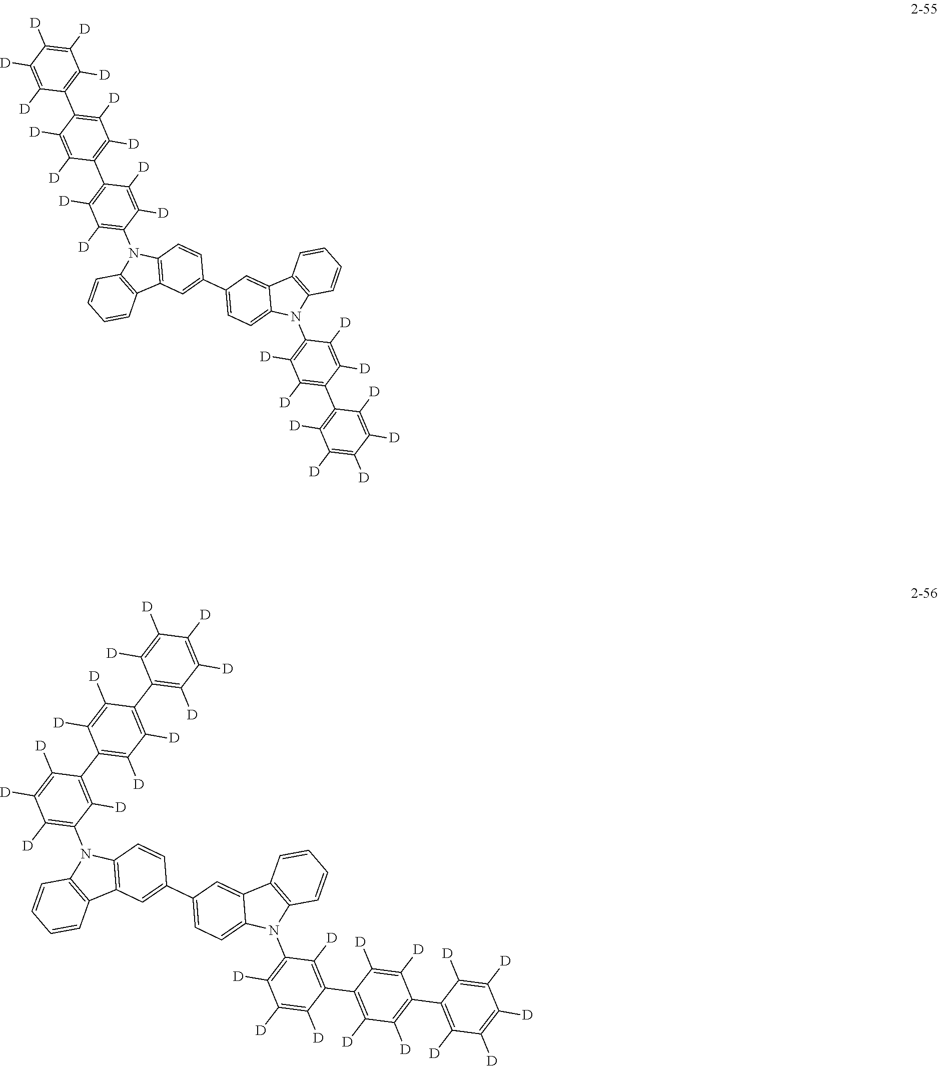

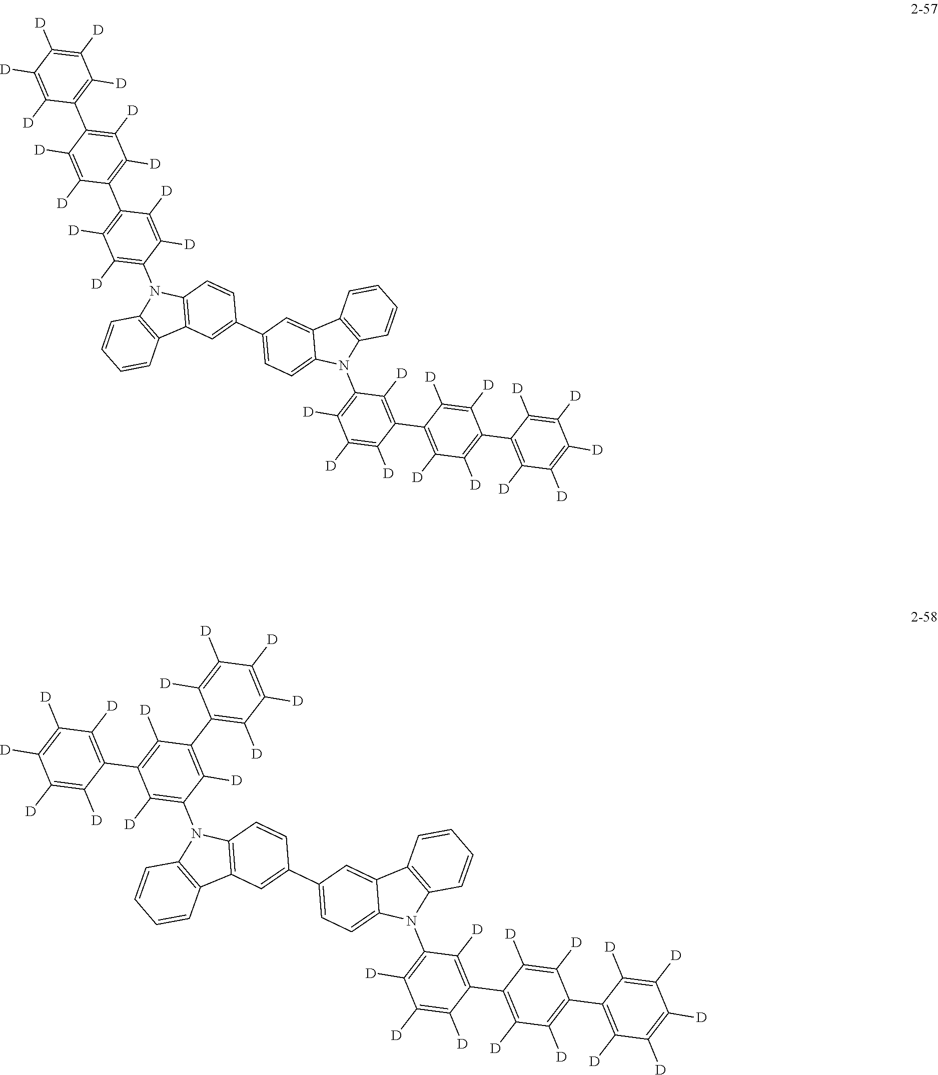

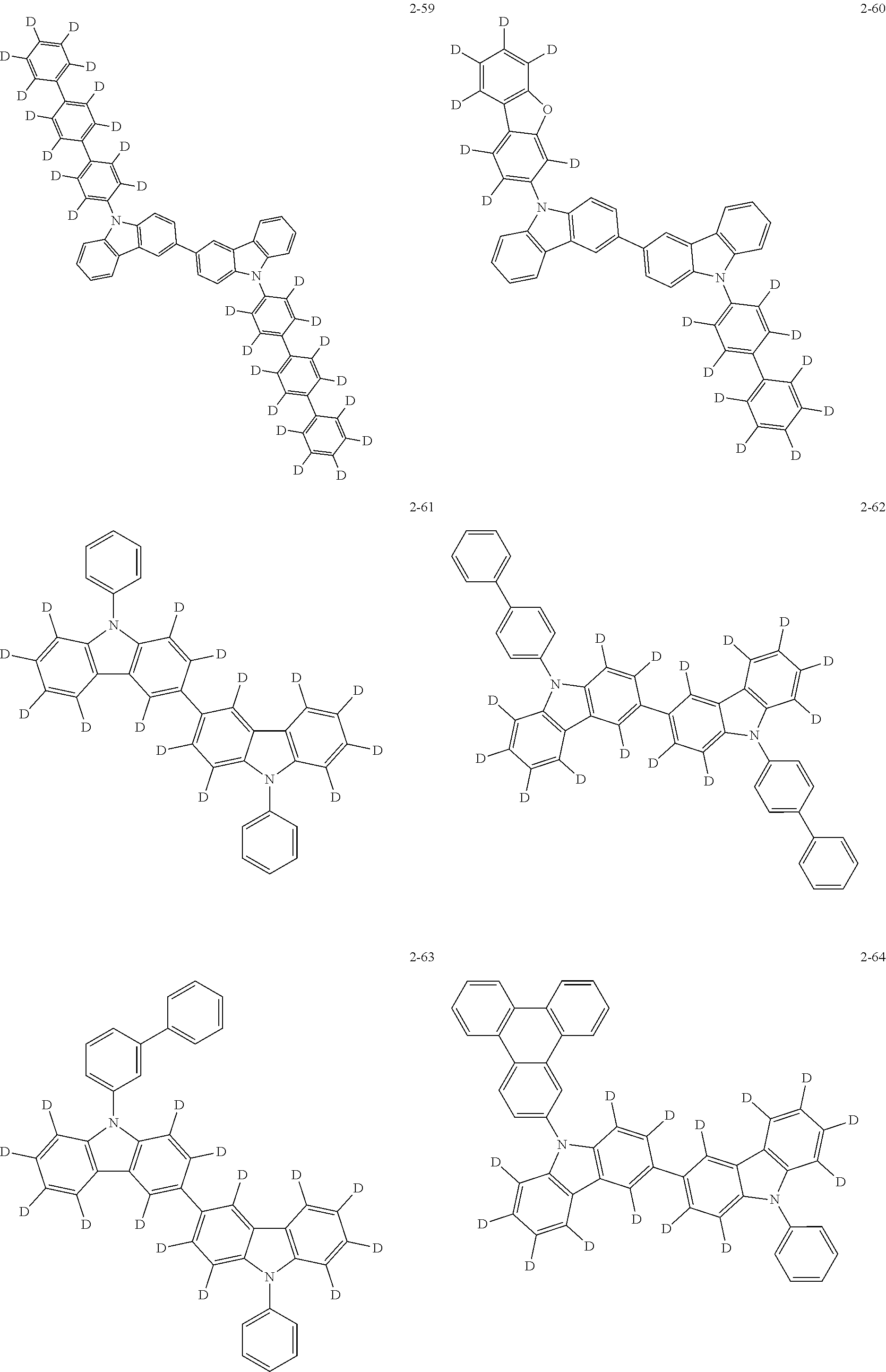

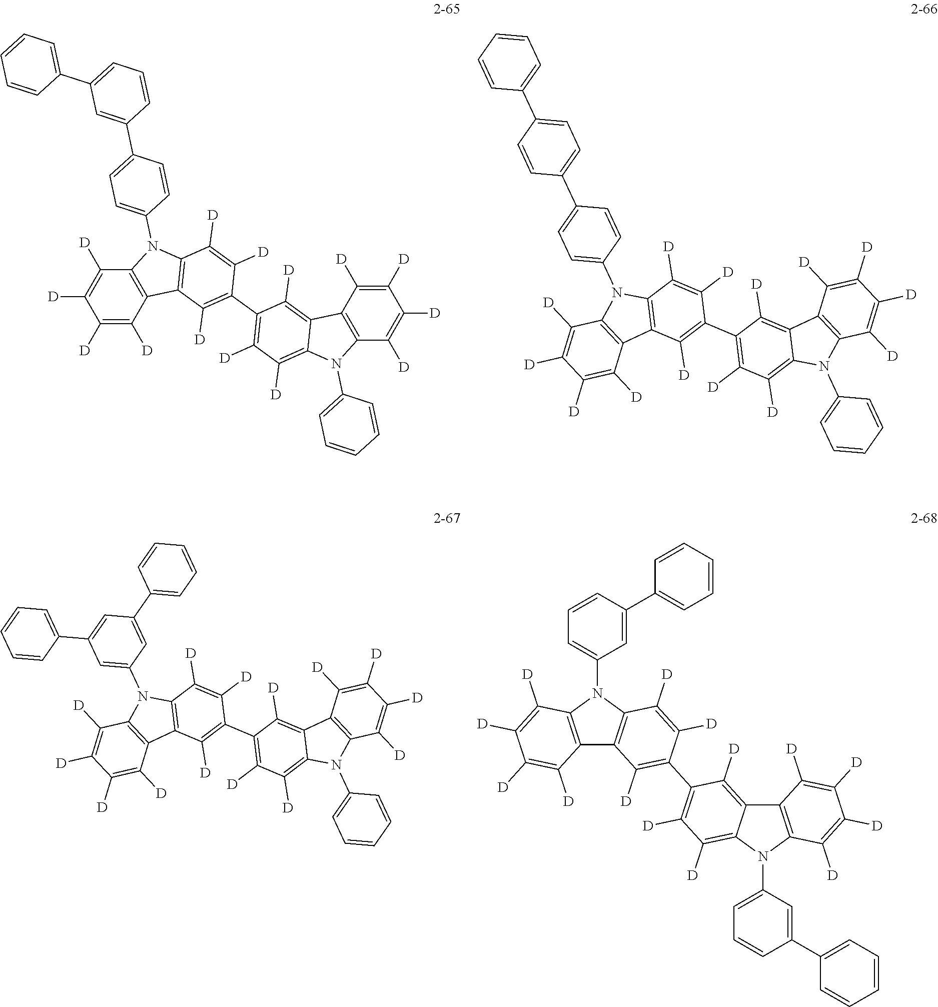

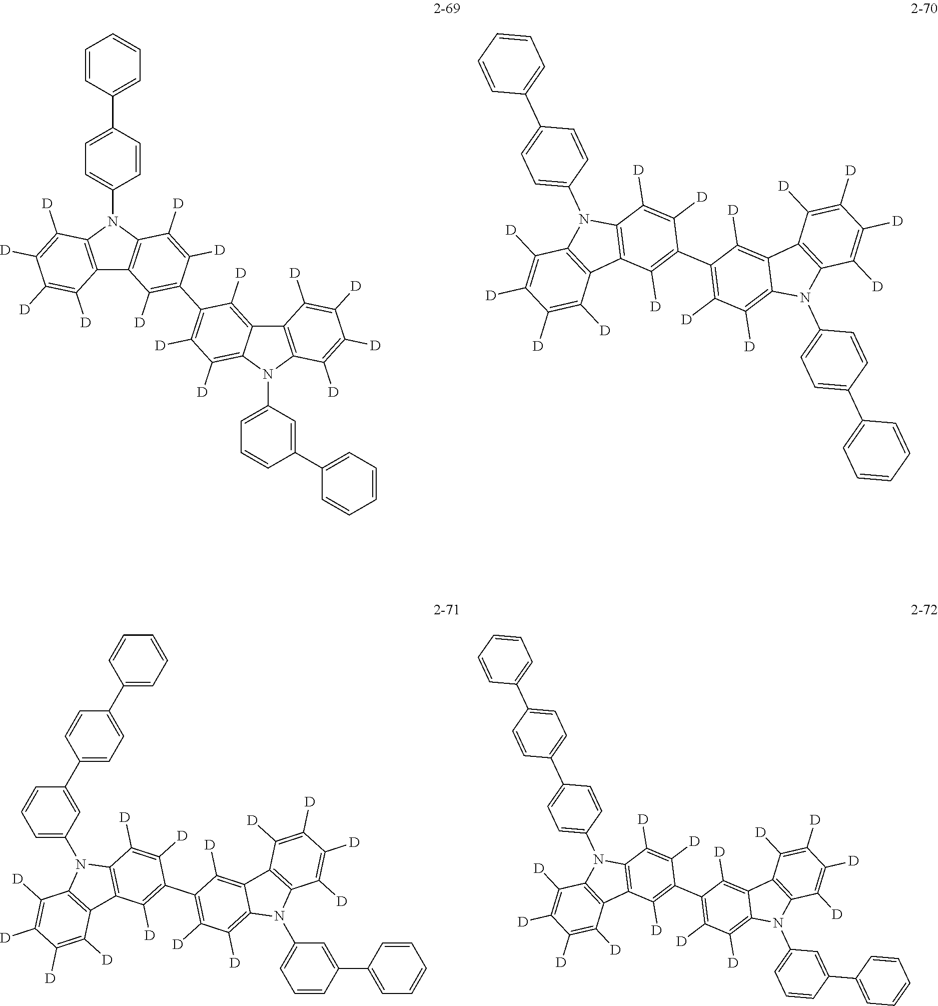

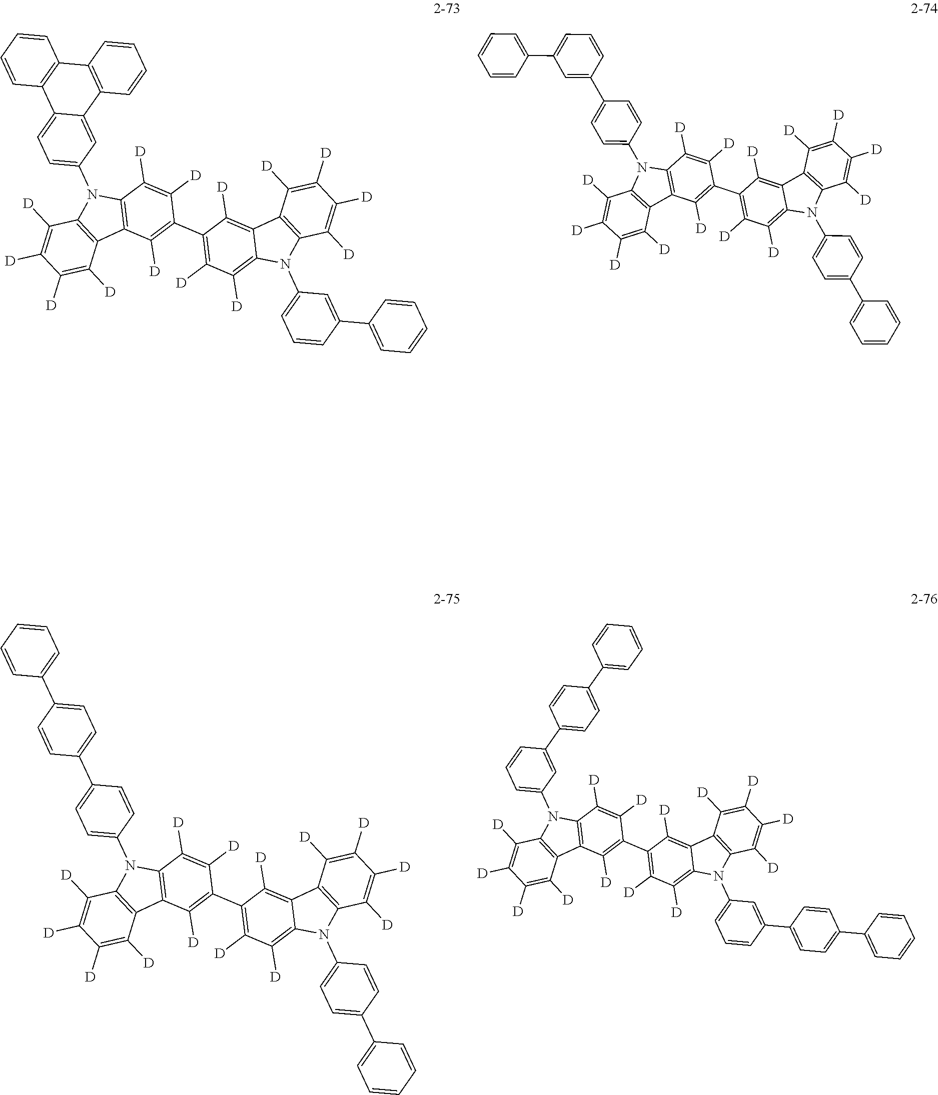

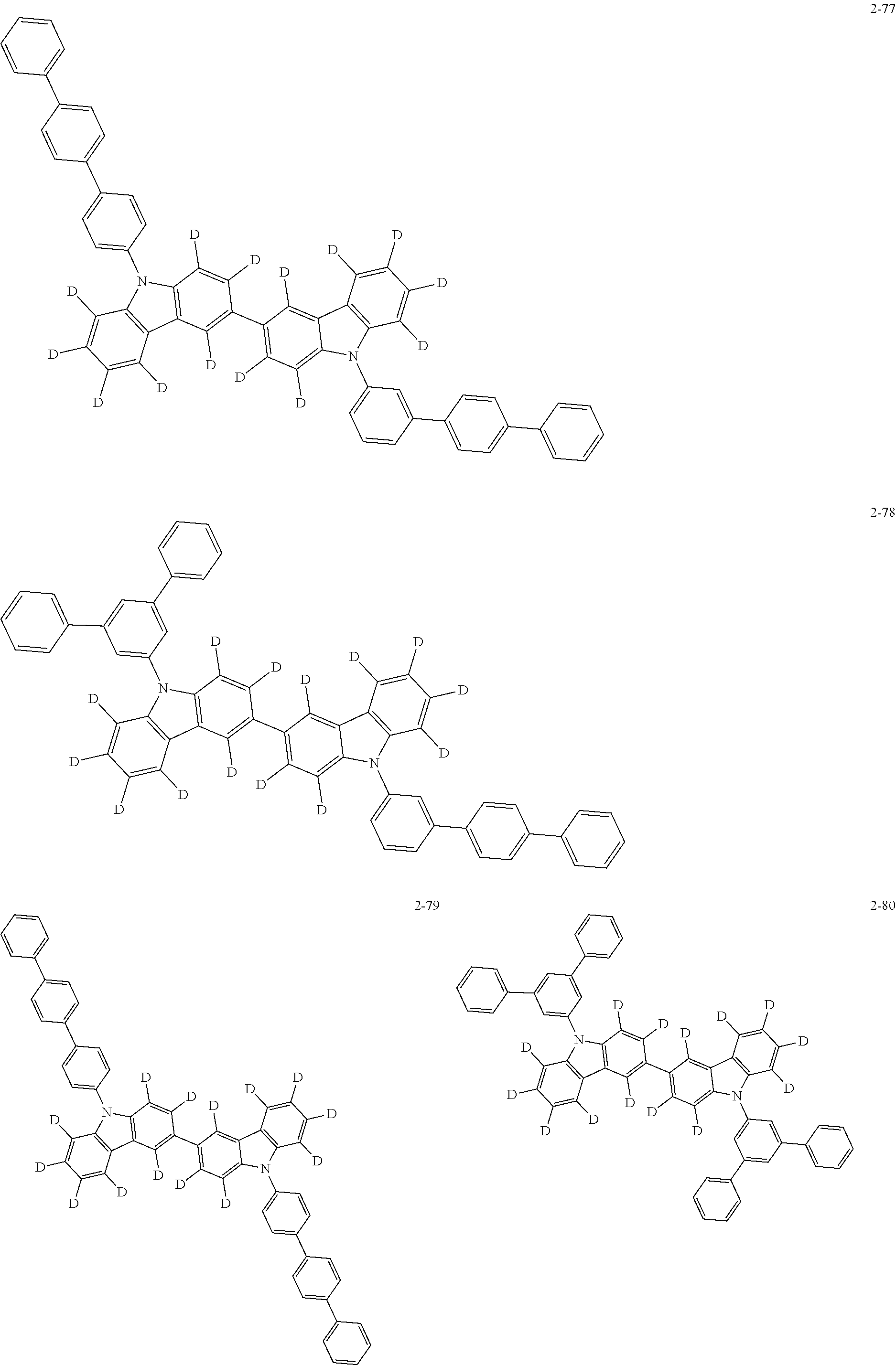

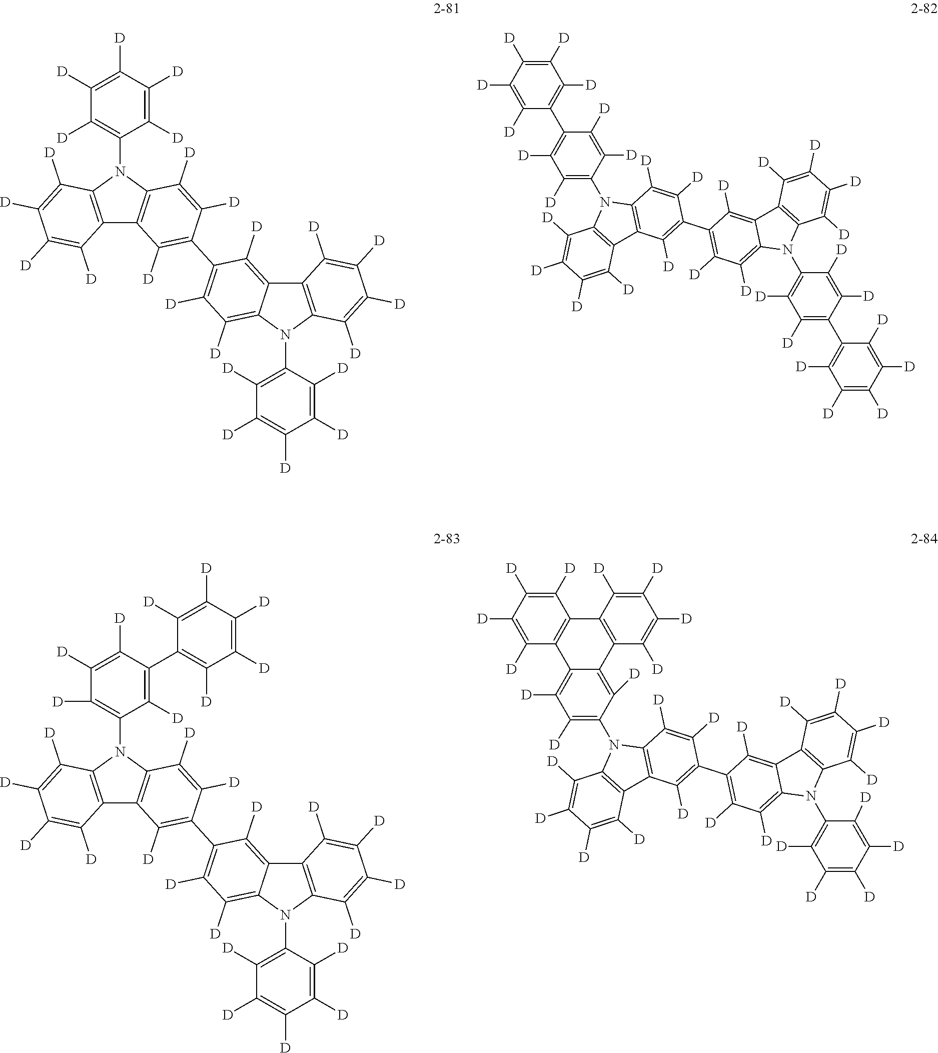

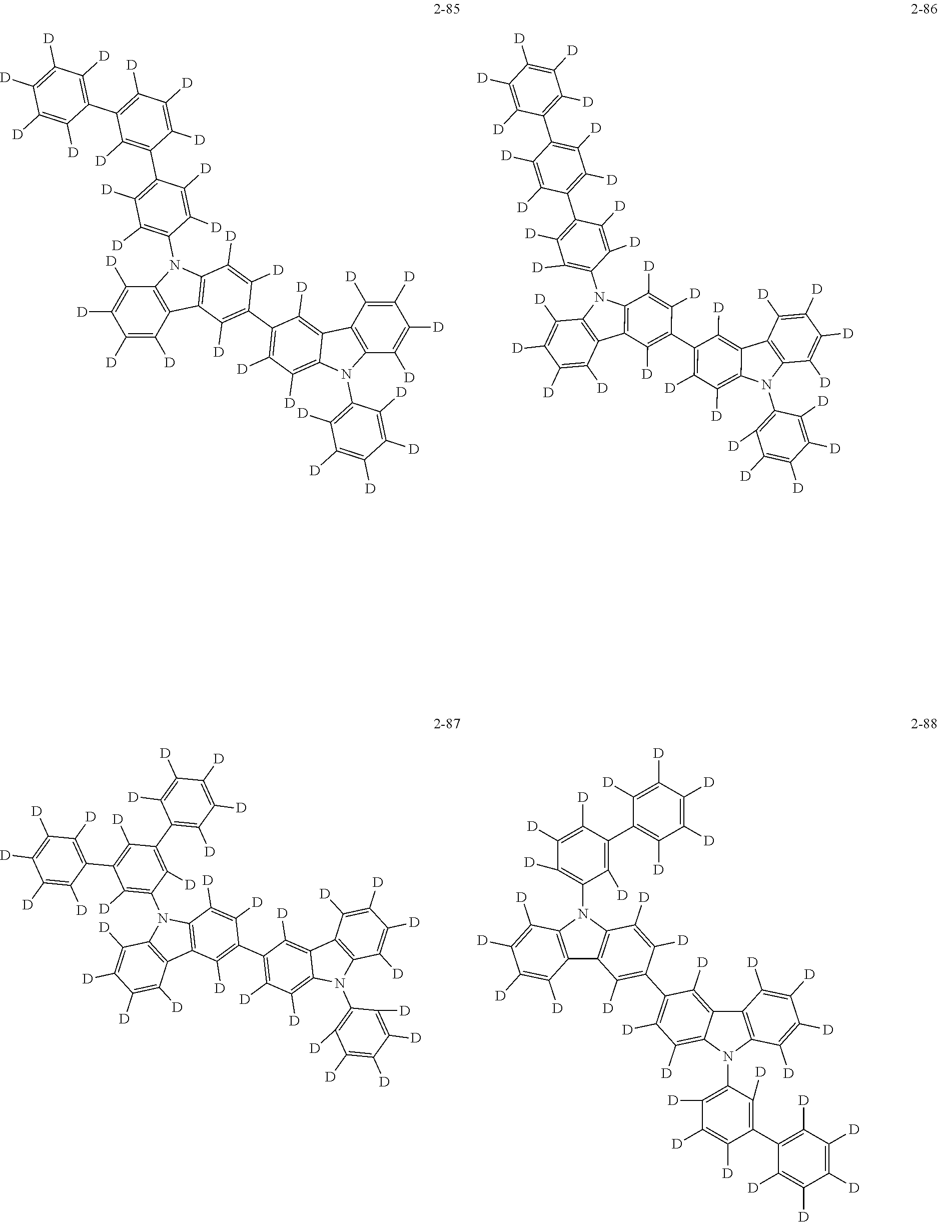

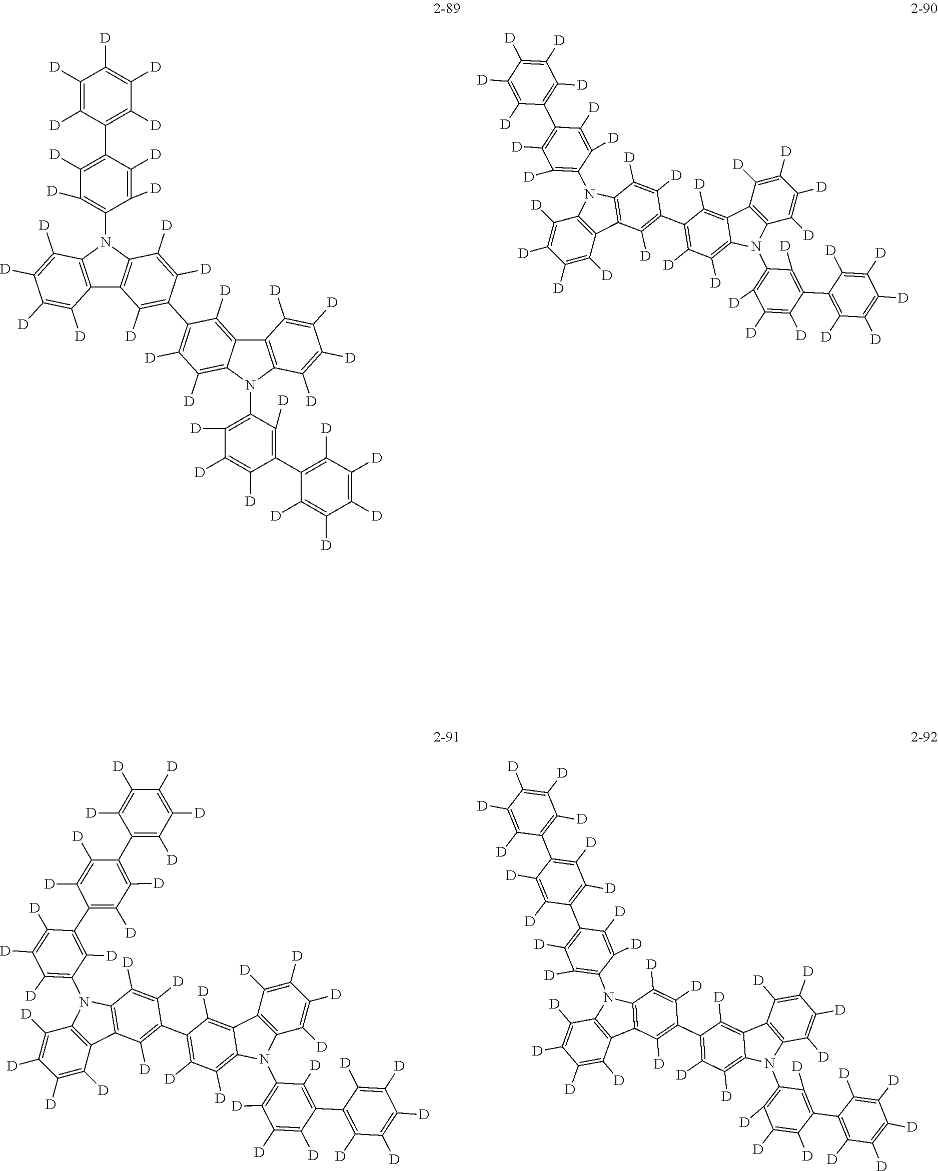

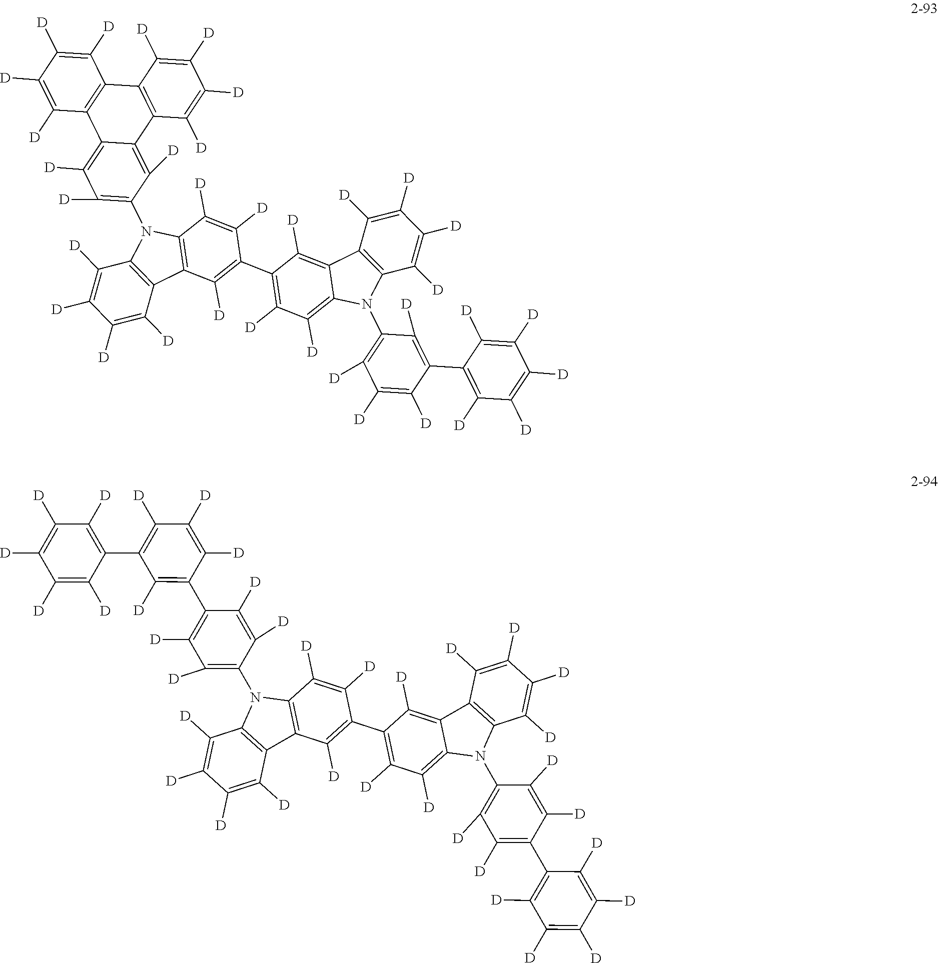

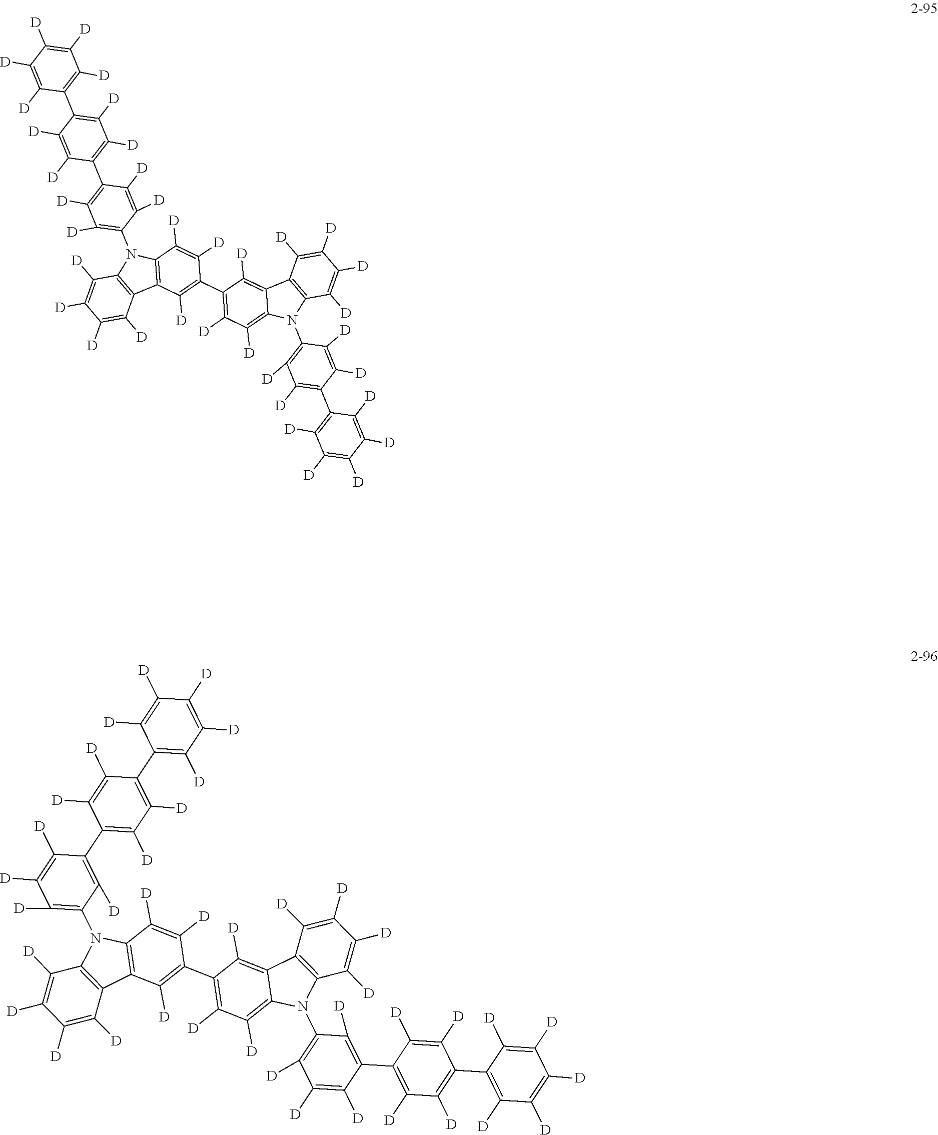

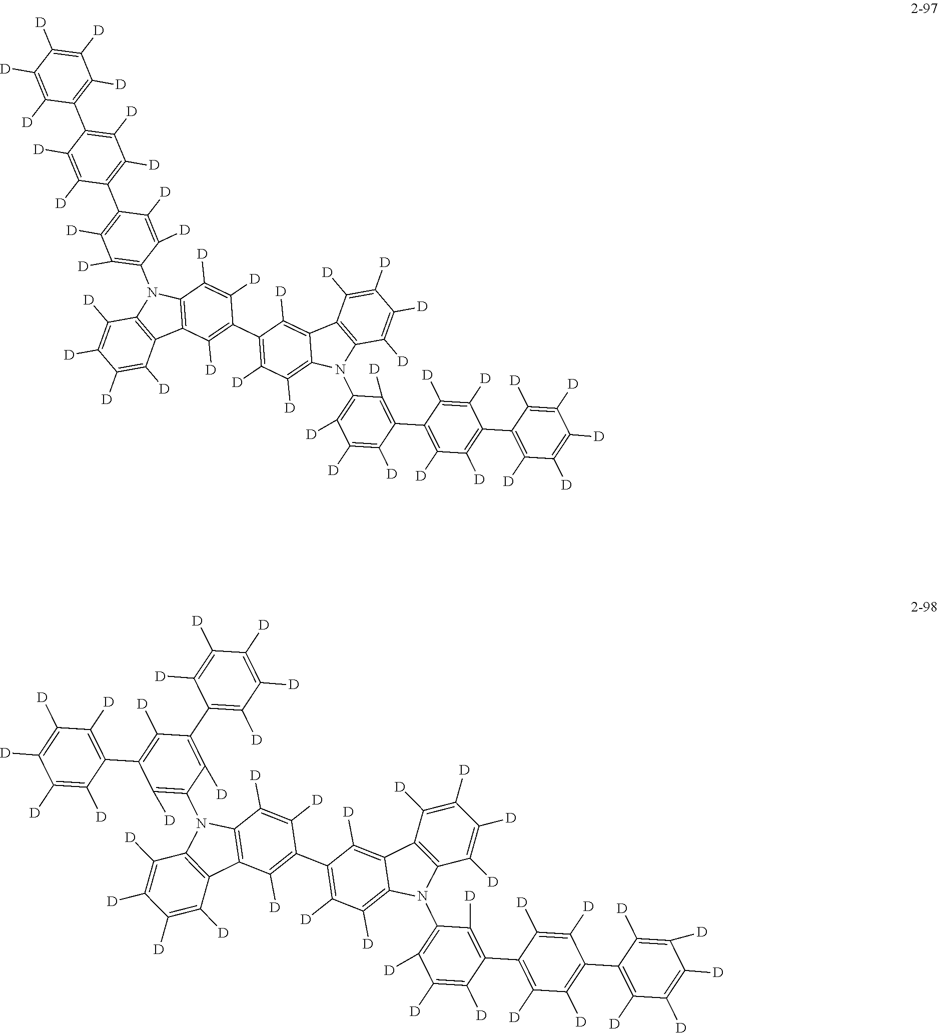

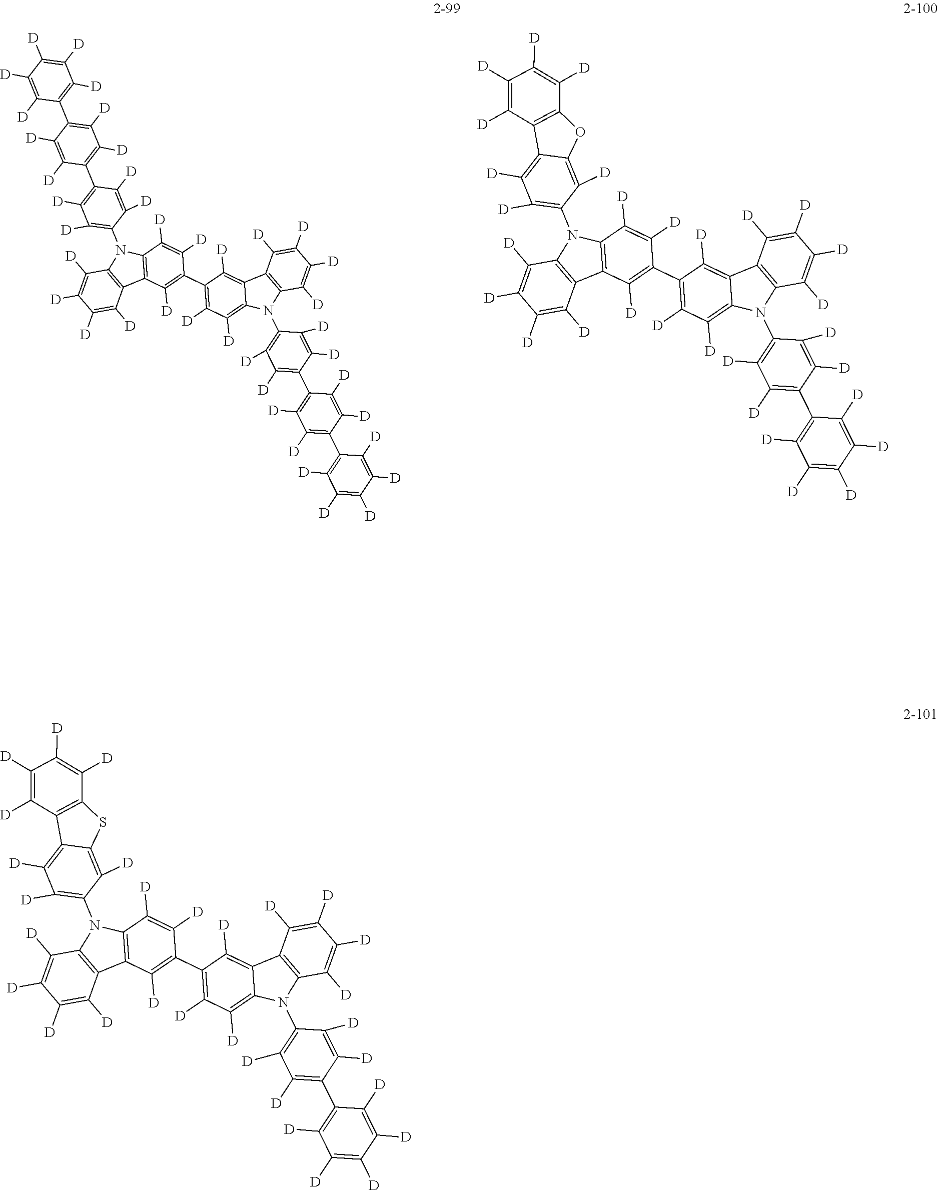

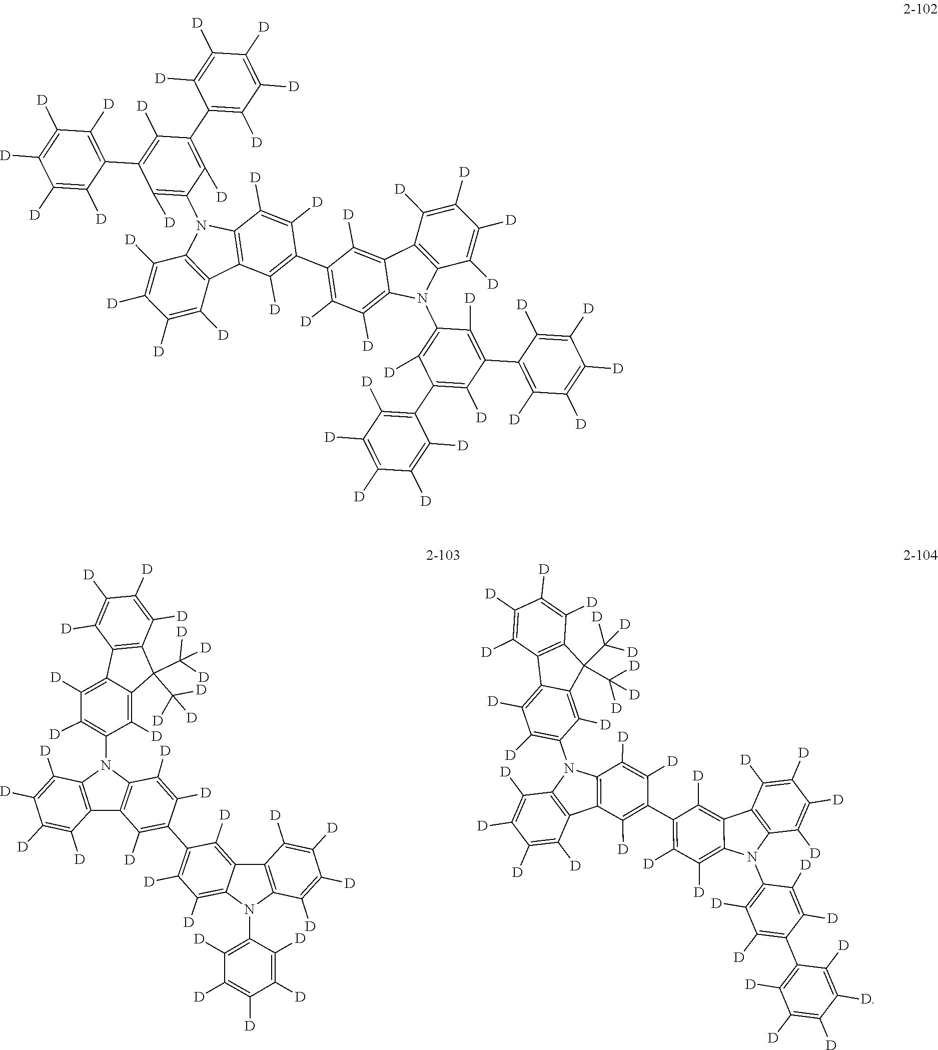

13. The organic light emitting device of claim 9, wherein the organic material layer comprises a light emitting layer, and the light emitting layer comprises the heterocyclic compound represented by Chemical Formula 1 and the heterocyclic compound represented by Chemical Formula 2.

14. The organic light emitting device of claim 9, wherein the organic material layer comprises a light emitting layer, and the light emitting layer comprises a host material, and the host material comprises the heterocyclic compound represented by Chemical Formula 1 and the heterocyclic compound represented by Chemical Formula 2.

15. The organic light emitting device of claim 8, further comprising one or two or more layers selected from the group consisting of a light emitting layer, a hole injection layer, a hole transport layer, an electron blocking layer, a hole blocking layer, an electron transport layer and an electron injection layer.

16. A composition for an organic material layer of an organic light emitting device, comprising the heterocyclic compound represented by Chemical Formula 1 according to claim 1, and a heterocyclic compound represented by the following Chemical Formula 2: 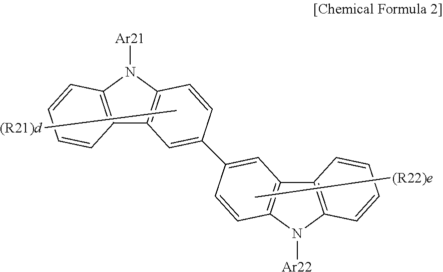
wherein, in Chemical Formula 2,
Ar21 and Ar22 are the same as or different from each other, and are each independently a substituted or unsubstituted C6 to C60 aryl group; or a substituted or unsubstituted C2 to C60 heteroaryl group,
R21 and R22 are the same as or different from each other, and are each independently selected from the group consisting of hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more adjacent groups are bonded to each other to form a substituted or unsubstituted aliphatic or aromatic hydrocarbon ring, and
d and e are an integer from 0 to 7, and when d and e are 2 or higher, substituents in the parenthesis are the same as or different from each other.

17. The composition of claim 16, wherein a weight ratio of the heterocyclic compound represented by Chemical Formula 1:the heterocyclic compound represented by Chemical Formula 2 in the composition is 1:10 to 10:1.
